(19) United States
(12) Patent Application Publication
(10) Pub. No.: US 2026/0001870 A1
(43) Pub. Date: Jan. 1, 2026

(54) INHIBITOR COMPOUNDS

(71) Applicant: Cincera Therapeutics Pty Ltd, Melbourne (AU)

(72) Inventors: Bernard Luke FLYNN, Donvale (AU); Giang LE, Templestowe Lower (AU); Shuxin YANG, Brunswick (AU)

(73) Assignee: Cincera Therapeutics Pty Ltd, Melbourne (AU)

(21) Appl. No.: 19/322,461

(22) Filed: Sep. 8, 2025

Related U.S. Application Data
(63) Continuation of application No. 17/629,218, filed on Jan. 21, 2022, now Pat. No. 12,448,371.

(30) Foreign Application Priority Data
Jul. 24, 2019 (AU) 2019902614

(51) Int. Cl. C07D 413/12; A61P 11/00; C07D 401/12; C07D 401/14; C07D 413/14; C07D 417/12

(52) U.S. Cl. CPC C07D 413/12; A61P 11/00; C07D 401/12; C07D 401/14; C07D 413/14; C07D 417/12

(57) ABSTRACT

The disclosure relates to heterocyclic compounds and methods for their preparation. The disclosure provides compounds that may have beneficial therapeutic activity in the treatment of a disease or condition mediated by excessive or otherwise undesirable Des1 and/or fibriotic activity.

The present application is continuation of U.S. application Ser. No. 17/629,218, filed Jan. 21, 2022, which is a national phase application under 35 U.S.C. § 371 of International Application No. PCT/AU2020/050763, filed Jul. 24, 2020, the entire contents of each of which are hereby incorporated by reference. International Application No. PCT/AU2020/050763 claims the priority benefit of Australian Application No. 2019902614, filed Jul. 24, 2019.

### FIELD

The present disclosure relates generally, but not exclusively, to compounds and their use in therapy, for example as enzyme interacting agents which interact with one or more enzymes in the sphingolipid biosynthesis pathway, e.g. dihydroceramide desaturase. The disclosure further relates to the use of such compounds as research tools, their use in therapy, to compositions and agents comprising said compounds, their manufacture, and to methods of treatment using said compounds.

### BACKGROUND

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Sphingolipids, a class of compounds defined by their common 18 carbon amino alcohol backbones, mediate cell-cell and cell-substratum interactions, modulate the behavior of cellular proteins and receptors, and participate in signal transduction. The sphingolipids are synthesised de novo from palmitoyl-CoA and serine via a pathway whereby the carbon backbone, alcohol and amino groups are modified to form the various bioactive compounds, such as dihydroceramide, ceramide, sphingosine and sphingosine-1-phosphate (Scheme 1). Perturbations in the sphingolipid biosynthetic pathway are implicated in many physiological and pathophysiological processes, including cancer, diabetes, fibrosis, inflammation, viral infection and Alzheimer's disease.

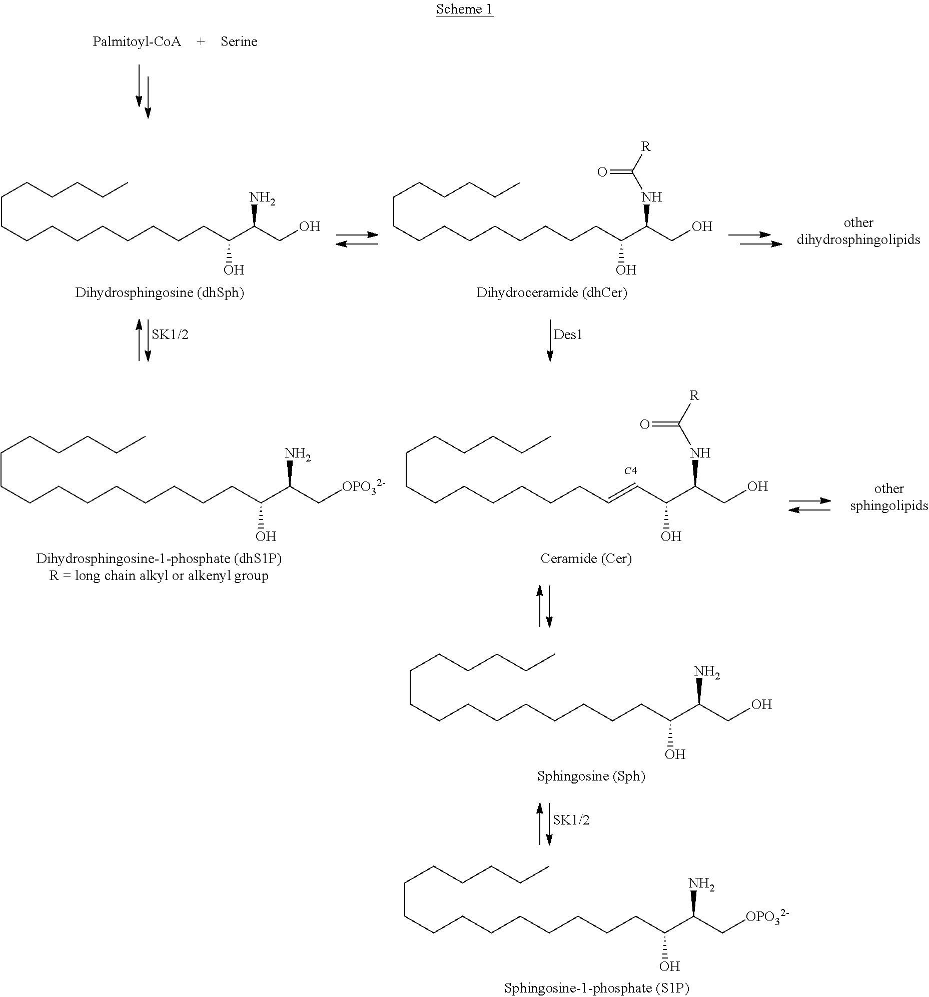

Dihydroceramide desaturase (Des), introduces a double-bond at C4 of the C18 backbone, converting dihydroceramides (dhCer) to ceramides (Cer). There are two isoforms of Des, Des1 and Des2. Des1 accounts for a major component of the Cer production in most tissues, whereas Des2 acts mostly as a C4-hydroxylase, converting Cer to phytoceramides. Whilst Des1 is found in most tissues, Des2 expression is confined mostly to the skin, intestines and kidneys. Since both dhCer and Cer are able undergo reversible conversion into other sphingolipids, including sphingosine (Sph) and sphingosine-1-phosphate (SIP), Des1 effectively controls the C4-saturation status of all sphingolipids. The activation status of Des1 controls the ratio of dihydro- and C4-unsaturated sphingolipids, which have different, and sometimes opposing, effects on cells function. Increased Des1 expression and/or activity increases the level of bioactive C4-unsaturated sphingolipids and has been linked to disease progression in a number of diseases, such as cancer, inflammation, fibrosis and metabolic disease. Accordingly, the use of Des1 inhibitors in drug therapy for the treatment of different diseases has been proposed, including cancer, inflammatory bowel disease (IBD), diabetes, non-alcoholic steatohepatitis (NASH), cystic fibrosis, heart failure, chronic kidney disease and viral and bacterial infection, amongst others (Gagliostro V et al. Prog. Lipid Res. 2012, 51. 82-94; Siddique, M. M. et al. J. Biol. Chem. 2015, 290, 15371-15379; Magaye, R., R. et al. Cell. Mol. Life Sci. 2019, 76, 1107-1134; Vieira, C. R. et al., Chem. Biol. 2010, 17, 766-775).

### SUMMARY

In one or more embodiments the disclosure provides compounds that have Des1 inhibitory activity. In one or more embodiments, the disclosure provides compounds that may have beneficial therapeutic activity in the treatment of a disease or condition mediated by excessive or otherwise undesirable Des1 and/or fibrotic activity.

In one aspect, the disclosure provides a compound of formula (I′):

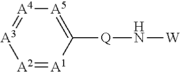

wherein:
A¹-A⁵ are independently selected from C—R^{a} and N, wherein any 0, 1, 2, 3 or 4 of A¹-A⁵ may be N;
each R^{a} is independently H or R^{aa}, wherein R^{aa} is selected from halo, alkyl, haloalkyl, cycloalkyl, halocycloalkyl, alkoxy, haloalkoxy, cycloalkoxy, cycloamino, halocycloamino, alkoxylalkyl, and alkoxyalkoxy;
Q is a 5-membered heteroaromatic ring having 2, 3 or 4 ring heteroatoms, at least one of which must be N, and the remaining are independently selected from N, O and S, and wherein a ring carbon atom bearing a hydrogen atom, or a ring nitrogen atom bearing a hydrogen atom, if present, may be optionally substituted with Q^{a}, which is selected from halo, haloalkyl and alkyl;
W is a 6-membered N-containing heterocycle selected from:

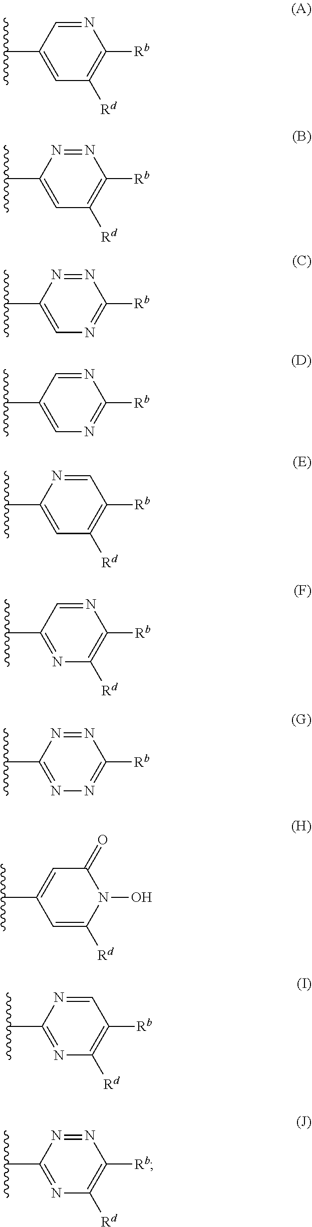

wherein
R^{b} is selected from:
—OH, provided that when R^{b} is OH, W is (E) or (I);
K—NR^{c}—Y, or a tautomer thereof,
—(CH₂)ₚ—NH—OH, and

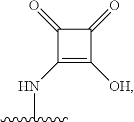

wherein
K is SO, SO₂, C(═X′) or NHC(═X′), where X′ is O or NH;
R^{c} is H, C₁₋₆ alkyl; or hydroxyC₁₋₆ alkyl;
Y is OH, NHR^{e} (wherein R^{e} is H, C₁₋₆ alkyl, —(C═O)H or —(C═O)C₁₋₆ alkyl), hydroxyC₁₋₆ alkyl or (SC₁₋₆ alkyl)C₁₋₆ alkyl; and
p is 0 or 1; and
R^{d} is selected from H, OH, halo, C₁₋₆ alkyl and C₁₋₆ alkoxy;
provided that the compound does not have the formula

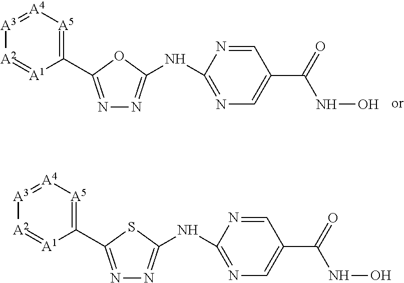

where any four of A¹-A⁵ are C—H, and the other is C—R^{a} where R^{a} is H, halo, CH₃ or OCH₃;
or a pharmaceutically acceptable salt or solvate thereof.

In another aspect, there is provided a compound of Formula (I″), wherein R^{b} and R^{d} of Formula (I′) are transposed, or a pharmaceutically acceptable salt or solvate thereof.

In some embodiments, the 5-membered heteroaromatic ring has 2 or 3 ring heteroatoms. In some embodiments Q is selected from

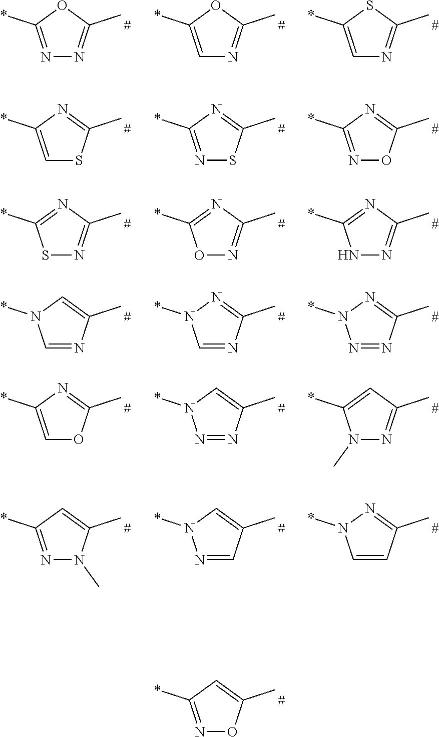

wherein # denotes the bond attached to NH and * denotes the bond attached to the aryl or heteroaryl ring defined by A¹-A⁵.

In some embodiments Q^{a} is selected from halo, C₁₋₆ alkyl (i.e. C₁ alkyl, C₂ alkyl, C₃ alkyl, C₄ alkyl, C₅ alkyl or C₆ alkyl), and haloC₁₋₆ alkyl (i.e. haloC₁ alkyl, haloC₂ alkyl, haloC₃ alkyl, haloC₄ alkyl, haloC₅ alkyl or haloC₆ alkyl).

In some embodiments, Each of A¹-A⁵ is C—R^{a}. In some further embodiments thereof one or two of C—R^{a} is C—R^{aa}, and the remainder C—H. In some further embodiments, at least A³ is C—R^{aa}.

In some embodiments, 0, 1, 2, 3 or 4 of A¹, A², A⁴ and A⁵ are N. In some embodiments thereof A³ is C—R^{aa}.

In some embodiments, at least A³ is C—R^{aa}. In some further embodiments A³ is C—R^{aa} and A¹, A², A⁴ and A⁵ are each C—H. In some embodiments A³ is C—R^{aa} and one of A⁴ or A² is the same or different C—R^{aa}. In some embodiments, any one or two of A¹, A², A³, A⁴ or A⁵ may be N. In some embodiments A³ is C—R^{aa} and A¹ or A⁵ is N. In further embodiments thereof, A³ is C—R^{aa}, A¹ or A⁵ is N and the remaining A are C—H.

In some embodiments, A⁵ or A¹ is N; or A² or A⁴ is N; or A¹ and A⁵ are both N; or A² and A⁴ are both N; or A¹ and A⁴, or A² and A⁵ are both N; or A¹ and A² or A⁴ and A⁵ are both N. In some of these embodiments A³ is C—R^{aa}.

In some embodiments, W contains 1 or 2 ring nitrogen atoms i.e. (A), (B), (D), (E), (F), (H) and (I). In some embodiments, where R^{b} is not OH, W is not a group of formula (E). In some embodiments, where R^{b} is not OH, W is not a group of formula (I). In some embodiments, where R^{b} is not OH, W is not a group of formula (I) or (E). In some embodiments, where R^{b} is not OH, W is a group having at least one N ring atom adjacent, or ortho- to the ring carbon atom bearing R^{b}.

In some embodiments, R^{b} is OH and W is (E) or (I).

In some embodiments, R^{b} is K—NR^{c}—Y, or a tautomer thereof, or —(CH₂)ₚ—NH—OH. In further embodiments, NHR^{e} is NH₂, NHC₁₋₃ alkyl, NHC(═O)H, NH(C(═O)C₁₋₃ alkyl.

In some embodiments, R^{b} is K—NR^{c}—Y, or a tautomer thereof, or —(CH₂)ₚ—NH—OH, and W is one of (A), (B), (C), (D), (F), (G), (H), (J). In further embodiments, NHR^{e} is NH₂, NHC₁₋₃ alkyl, NHC(═O)H, NH(C(═O)C₁₋₃ alkyl.

R^{d} may be selected from H, OH, F, Cl, Br, I, C₁ alkyl, C₂ alkyl, C₃ alkyl, C₄ alkyl, C₅ alkyl, C₆ alkyl, C₁ alkoxy, C₂ alkoxy, C₃ alkoxy, C₄ alkoxy C₅ alkoxy, C₆ alkoxy. In some further embodiments, R^{d} is H, OH, F, Cl, Br, I, CH₃ or OCH₃.

In another aspect, the disclosure provides a composition comprising a compound of Formula (I′) and/or (I″), or a pharmaceutically acceptable salt or solvate thereof, and a pharmaceutically acceptable additive.

The disclosure also provides a compound of Formula (I′) and/or (I″), or a pharmaceutically acceptable salt or solvate thereof, or a composition comprising said compound or a pharmaceutically acceptable salt or solvate thereof, for use as an agent for inhibiting or otherwise interacting with Des1.

The disclosure also provides a compound of Formula (I′) and/or (I″), or a pharmaceutically acceptable salt or solvate thereof, or a composition comprising said compound or a pharmaceutically acceptable salt or solvate thereof, for use in therapy, such as treating a disease or condition in which Des1 inhibition is beneficial, and/or for treating fibrosis or a fibrotic disease.

A further aspect disclosed herein provides a method of treating a disease or condition in which Des1 inhibition is beneficial, in a subject in need thereof, comprising administering to said subject, a compound of Formula (I′) and/or (I″), or a pharmaceutically acceptable salt or solvate thereof.

In some embodiments, the disease or condition is a proliferative, inflammatory or fibrotic disease

Yet another aspect disclosed herein provides use of a compound of Formula (I′) and/or (I″), or a pharmaceutically acceptable salt or solvate thereof, in the manufacture of a medicament for treating a disease in which Des1 inhibition is beneficial.

Yet another aspect disclosed herein provides use of a compound of Formula (I′) and/or (I″), or a pharmaceutically acceptable salt or solvate thereof, in the manufacture of a medicament for treating a fibrosis or a fibrotic disease.

Yet another aspect disclosed herein provides a method of treating fibrosis or a fibrotic disease in a subject in need thereof, comprising administering to said subject, a compound of Formula (I′) and/or (I″), or a pharmaceutically acceptable salt or solvate thereof.

In some embodiments, the compounds disclosed herein may also be useful as research tools, for example in the investigation of the role and activity of Des1, and/or as candidate, comparison or control molecules in an assay or model for Des1 activity or its inhibition, and/or as candidate, comparison or control molecules in an assay or model for one or more potential therapeutic applications, such anti-fibrotic activity for the prevention or treatment of fibrosis.

### FIGURES

FIG. 1 graphically depicts proline incorporation in renal mesangial cells stimulated with TGF-β1 (5 ng/ml) in the presence or absence of Compound 8 (FIG. 1A) and Compound 46 (FIG. 1B) at 0.01, 0.1, 3 and 10 μM. Data is represented as mean+/−standard deviation of raw ³H-proline counts per minute normalised to micrograms of protein. Data is from 3 independent experiments. One way ANOVA with Tukeys pos hoc analysis to correct for multiple differences. ***, ****=p<0.001 & 0.0001 compared to zero control. ##, ###, ####=p<0.01, 0.001 &0.0001 compared to TGF-β1 alone.

FIG. 2 represents the concentration-dependent inhibition of TGF-β 1-mediated αSMA expression in IPF donors in the presence of Compound 8 (FIG. 2A) and Compound 46 (FIG. 2B). The graphs display normalised data for percentage of inhibition (PIN) and remaining cells (%).

### DESCRIPTION

Throughout this specification and the claims which follow, unless the context requires otherwise, the word “comprise” and variations such as “comprises” and “comprising” will be understood to imply the inclusion of a stated integer or step or group of integers but not the exclusion of any other integer or step or group of integers or steps.

Throughout this specification and the claims which follow, unless the context requires otherwise, the phrase “consisting essentially of”, and variations such as “consists essentially of” will be understood to indicate that the recited element(s) is/are essential i.e. necessary, elements of the invention. The phrase allows for the presence of other non-recited elements which do not materially affect the characteristics of the invention but excludes additional unspecified elements which would affect the basic and novel characteristics of the method defined.

All aspects, embodiments and examples described herein are encompassed and contemplated by the term “invention”.

The singular forms “a”, “an” and “the” as used throughout are intended to include plural aspects where appropriate unless the context clearly dictates otherwise.

Unless the context indicates otherwise, features described below may apply independently to any aspect or embodiment of the invention

As used herein, the term “halo” (or “halogen”) denotes fluoro (fluorine), chloro (chlorine), bromo (bromine) or iodo (iodine).

As used herein, the term “alkyl” (or “alk”), as used alone or in a composite term such as alkoxy, haloalkyl etc) denotes saturated straight chain, or branched alkyl, preferably C₁₋₂₀ alkyl, e.g. C₁₋₁₀ or C₁₋₆. Examples of straight chain and branched alkyl include methyl, ethyl, n-propyl, isopropyl, n-butyl, sec-butyl, t-butyl, n-pentyl, 1,2-dimethylpropyl, 1,1-dimethyl-propyl, hexyl, 4-methylpentyl, 1-methylpentyl, 2-methylpentyl, 3-methylpentyl, 1,1-dimethylbutyl, 2,2-dimethylbutyl, 3,3-dimethylbutyl, 1,2-dimethylbutyl, 1,3-dimethylbutyl, 1,2,2,-trimethylpropyl, 1,1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethylpentyl, 1,2,3-trimethylbutyl, 1,1,2-trimethylbutyl, 1,1,3-trimethylbutyl, octyl, 6-methylheptyl, 1-methylheptyl and 1,1,3,3-tetramethylbutyl. Where an alkyl group is referred to generally as “propyl”, butyl” etc, it will be understood that this can refer to any of straight or branched isomers where appropriate. An alkyl group, either alone or as part of an alkoxy, haloalkyl or haloalkoxy etc group as defined for R^{a} may be unsubstituted or substituted by one or more (e.g. 1, 2, 3, 4, 5 etc, as permitted), same or different, optional substituents.

The term “cycloalkyl” includes any of non-aromatic monocyclic, bicyclic and polycyclic, (including fused or bridged) hydrocarbon residues, e.g. C₃₋₂₀ (such as C₃₋₁₀ or C₃₋₈ or C₃₋₆) monocyclic 5-6-membered or bicyclic 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl and decalinyl. A cycloalkyl group may be optionally substituted by one or more optional substituents as herein defined. In some embodiments, a monocycloalkyl group may be substituted by a bridging group to form a bicyclic bridged group.

“Halocycloalkyl” refers to a cycloalkyl group, as herein defined, independently substituted one or more times with one or more, same or different halogen atoms. One or more carbon atoms (e.g. 1, 2, or more) are independently substituted with one or more halogen atoms. In some embodiments, two hydrogen atoms attached to any one carbon ring atom are replaced by the same or different halogen atom. In some embodiments one hydrogen atom attached to any one carbon ring atom is replaced by a halogen atom. Some non-limiting examples include chloro-, iodo-, fluoro or bromo-cyclopropyl, cyclobutyl, cyclopentyl and cyclohexyl, and dichloro-, diiodo-, difluoro or dibromo-cyclopropyl, cyclobutyl, cyclopentyl and cyclohexyl.

“Cycloalkoxy” when used alone or in a composite term denotes cycloalkyl, as herein defined, when linked by an oxygen atom. Some non-limiting examples include: OC₃₋₆ cycloalkyl (e.g. cyclopropyloxy, cyclobutyloxy, cyclopentyloxy and cyclohexyloxy).

The term “cycloamino” refers to a cycloalkyl group as herein defined wherein a carbon atom is replaced by a nitrogen atom. The cycloamino group may be connected via a carbon ring atom or a nitrogen ring atom. In some embodiments, the carbon atom connecting the group is replaced by nitrogen, i.e. the cycloamino group is connected through the nitrogen atom. Some exemplary groups include, 3, 4, 5, and 6-membered rings, e.g. aziridinyl, azetidinyl, pyrrolidinyl, and piperidinyl. In embodiments where the cycloamino group is attached via a carbon atom, the ring nitrogen may be unsubstituted or may be i substituted with one or two, same or different, C₁₋₆ alkyl groups, e.g., C₁ alkyl, C₂ alkyl, C₃ alkyl, C₄ alkyl, C₅ alkyl, C₆ alkyl.

The term “halocycloamino” refers to a cycloamino group independently substituted one or more times with one or more, same or different halogen atoms. One or more carbon atoms (e.g. 1, 2, or more) are independently substituted with one or more halogen atoms. In some embodiments, all hydrogen atoms attached to any one carbon atom are replaced by the same or different halogen atom. In some embodiments, two hydrogen atoms attached to any one carbon atom are replaced by the same or different halogen atom. In some embodiments one hydrogen atom attached to any one carbon atom is replaced by a halogen atom. Some non-limiting examples include chloro-, iodo-, fluoro or bromo-aziridinyl, azetidinyl, pyrrolidinyl, and piperidinyl, and dichloro-, diiodo-, difluoro or dibromo-aziridinyl, azetidinyl, pyrrolidinyl, and piperidinyl.

“Alkoxy” when used alone or in a composite term denotes alkyl, as herein defined, when linked by an oxygen atom. Some non-limiting examples include: OC₁₋₆ alkyl (e.g. OMe, OEt, On-Pr, Oi-Pr, On-Bu, Oi-Bu, Ot-Bu).

“Haloalkyl” refers to an alkyl group, as herein defined, independently substituted one or more times with one or more, same or different halogen atoms. Where “alkyl” comprises more than one carbon atom, some (e.g. 1, 2, or more) or all of the carbon atoms are independently substituted with one or more halogen atoms. In some embodiments, all hydrogen atoms attached to any one carbon atom are replaced by the same or different halogen atom. In some embodiments, two hydrogen atoms attached to any one carbon atom are replaced by the same or different halogen atom. In some embodiments one hydrogen atom attached to any one carbon atom is replaced by a halogen atom. The alkyl group may be straight chained or branched. Some non-limiting examples of “haloalkyl” include haloC₁₋₆ alkyl, such as: —(CH₂)_{q}CF₃, —(CH₂)_{q}CCl₃, —(CH₂)_{q}CBr₃, —(CH₂)_{q}CHF₂, —(CH₂)_{q}CHCl₂, —(CH₂)_{q}CHBr₂, —(CH₂)_{q}CH₂F, —(CH₂)_{q}CH₂Cl, and —(CH₂)_{q}CH₂Br, where q is 0, 1, 2, 3, 4 or 5).

“Haloalkoxy” refers to a haloalkyl group, as defined above, when linked by an oxygen atom. Some non-limiting examples include O(haloC₁₋₆ alkyl) such as: —O(CH₂)_{q}CF₃, —O(CH₂)_{q}CCl₃, —O(CH₂)_{q}CBr₃, —O(CH₂)_{q}CHF₂, —O(CH₂)_{q}CHCl₂, and —O(CH₂)_{q}CHBr₂, —O(CH₂)_{q}CH₂F, —O(CH₂)_{q}CH₂Cl, and —O(CH₂)_{q}CH₂Br, where q is 0, 1, 2, 3, 4 or 5).

“Alkoxyalkyl” refers to an alkyl group, as herein defined, independently substituted one or more times with an alkoxy group. Where “alkyl” comprises more than one carbon atom, some (e.g. 1, 2, or more) or all of the carbon atoms may be independently substituted with one or more same or different alkoxy groups. The alkyl group may be straight chained or branched and the alkoxy group may be straight chained or branched. Some non-limiting examples of “alkoxyalkyl” include C₁₋₆ alkoxyC₁₋₆ alkyl, including C₁₋₃ alkoxyC₁₋₆ alkyl, C₁₋₆ alkoxyC₁₋₃ alkyl, and C₁₋₃ alkoxyC₁₋₃ alkyl. Some further non-limiting examples include: —(CH₂)_{q}O(CH₂)ₜH (where q is 1, 2, 3, 4 5 or 6 and for any value of q, t is 1, 2, 3, 4, 5, or 6).

“Alkoxyalkoxy” refers to an alkoxy group, as herein defined, independently substituted one or more times with an alkoxy group. Some non-limiting examples include C₁₋₆ alkoxy (e.g. C₁₋₃ alkoxy) independently substituted one or more times with the same or different C₁₋₆ alkoxy group (e.g. C₁₋₃ alkoxy). Some non-limiting examples include: —OCH₂OCH₃, —O(CH₂)₂OCH₃, —O(CH₂)₃OCH₃, —OCH₂OCH₂CH₃, —O(CH₂)₂OCH₂CH₃, —O(CH₂)₃OCH₂CH₃, —OCH₂O(CH₂)₂CH₃, —O(CH₂)₂O(CH₂)₂CH₃, —O(CH₂)₃O(CH₂)₂CH₃.

“Hydroxyalkyl” refers to an alkyl group, as herein defined, independently substituted one or more times (e.g. 1, 2 or 3 times) with a hydroxy group. Some non-limiting examples include hydroxyC₁₋₆ alkyl, i.e. C₁₋₆ alkyl substituted one or more times (e.g. 1, 2 or 3 times) with a hydroxyl group. The alkyl group may be straight chained or branched. In further examples hydroxyalkyl refers to hydroxyC₁₋₃ alkyl, i.e. C₁₋₃ alkyl substituted one or more times (e.g. 1, 2 or 3 times) with a hydroxy group. Some non-limiting examples include: —(CH₂)OH, —(CH₂)₂OH, —CH(OH)CH₂OH, CH(OH)CH₃, —(CH₂)₃OH, —CH(OH)(CH₂)₂OH, —CH₂CH(OH)CH₂OH, —(CH(OH))₂CH₃ and —(CH(OH))₂CH₂OH.

“(SC₁₋₆ alkyl)C₁₋₆ alkyl” refers to C₁₋₆ alkyl group, as herein defined, independently substituted one or more times (e.g. 1, 2 or 3 times) with a —SC₁₋₆ alkyl group. Some non-limiting examples include C₁₋₃ alkyl substituted one or more times (e.g. 1, 2 or 3 times) with a —SC₁₋₆ alkyl group. The alkyl group may be straight chained or branched. In further examples—Some non-limiting examples include: —(CH₂)SCH₃, —(CH₂)₂SCH₃, —CH(SCH₃)CH₂SCH₃, CH(SCH₃)CH₃, —(CH₂)₃SCH₃, —CH(SCH₃)(CH₂)₂SCH₃, —CH₂CH(SCH₃)CH₂SCH₃, —(CH(SCH₃))₂CH₃ and —(CH(SCH₃))₂CH₂SCH₃.

As defined herein, a group may be optionally substituted, ie it may be unsubstituted or further substituted by one or more, same or different optional substituents. Optional substituents (which may be further substituted where indicated below) for “alkyl” or “alk”, either used alone or in a composite term, or by reference to in the definition of a term, for example cycloalkyl or cycloamino, include:
alkyl, (e.g. C₁₋₆ alkyl such as methyl, ethyl, propyl, butyl),
cycloalkyl (e.g. C₃₋₆cycloalkyl, such as cyclopropyl, cyclobutyl, cyclopentyl or cyclohexyl),
hydroxyalkyl (e.g. hydroxyC₁₋₆ alkyl, such as hydroxymethyl, hydroxyethyl, hydroxypropyl),
alkoxyalkyl (e.g. C₁₋₆ alkoxyC₁₋₆ alkyl, such as methoxymethyl, methoxyethyl, methoxypropyl, ethoxymethyl, ethoxyethyl, ethoxypropyl),
alkoxy (e.g. C₁₋₆ alkoxy, such as methoxy, ethoxy, propoxy, butoxy),
alkoxyalkoxy (e.g. C₁₋₆ alkoxyC₁₋₆ alkoxy, such as methoxymethoxy, methoxyethoxy, methoxypropoxy, ethoxymethoxy, ethoxyethoxy, ethoxypropoxy, propoxymethoxy, propoxyethoxy, propoxypropoxy),
cycloalkoxy (e.g. cyclopropoxy, cyclobutoxy, cyclopentoxyl, cyclohexyloxy), halo,
haloalkyl (which includes, mono-, di-, and trihalo, e.g. haloC₁₋₆ alkyl, such as trifluoromethyl, trichloromethyl, tribromomethyl),
haloalkoxy (which includes, mono-, di-, and trihalo, e.g. haloC₁₋₆ alkoxy, such as trifluoromethoxy, trichloromethoxy, tribromomethoxy),
hydroxy,
thiol (—SH),
alkylthio (e.g. —SC₁₋₆ alkyl),
phenyl (which itself may be further substituted e.g., by one or more of C₁₋₆ alkyl, halo, hydroxy,
hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)(C₁₋₆ alkyl)),
benzyl (wherein benzyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl,
—NHC(O)C₁₋₆ alkyl and —N(C₁₋₆ alkyl)(C₁₋₆ alkyl)),
phenoxy (wherein phenyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)(C₁₋₆ alkyl)),
benzyloxy (wherein benzyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl,
—NHC(O)C₁₋₆ alkyl and —N(C₁₋₆ alkyl)(C₁₋₆ alkyl)),
—NH₂,
alkylamino (e.g. —NHC₁₋₆ alkyl, such as methylamino, ethylamino, propylamino etc), dialkylamino (e.g. —NH(C₁₋₆ alkyl)₂, such as dimethylamino, diethylamino, dipropylamino), acylamino (e.g. —NHC(O)C₁₋₆ alkyl, such as —NHC(O)CH₃),
phenylamino (i.e. —NHphenyl, wherein phenyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)C₁₋₆ alkyl),
nitro,
cyano,
formyl,
acyl, including —C(O)-alkyl (e.g. —C(O)C₁₋₆ alkyl, such as acetyl),
—O—C(O)-alkyl (e.g. —OC(O)C₁₋₆ alkyl, such as acetyloxy),
benzoyl (wherein benzyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂,
—NHC₁₋₆ alkyl, —NHC(O)C₁₋₆ alkyl and —N(C₁₋₆ alkyl)(C₁₋₆ alkyl)), benzoyloxy (wherein benzyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)(C₁₋₆ alkyl)),
CO₂H,
CO₂ alkyl (e.g. CO₂C₁₋₆ alkyl such as methyl ester, ethyl ester, propyl ester, butyl ester), CO₂ phenyl (wherein phenyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)(C₁₋₆ alkyl)),
CO₂ benzyl (wherein benzyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)(C₁₋₆ alkyl)),
—CONH₂,
—C(O)NHphenyl (wherein phenyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)(C₁₋₆ alkyl)),
—C(O)NHbenzyl (wherein benzyl itself may be further substituted e.g., by one or more of C₁₋₆ alkyl, halo, hydroxy, hydroxyC₁₋₆ alkyl, C₁₋₆ alkoxy, C₁₋₆ alkoxyC₁₋₆ alkyl, C₁₋₆ alkoxyC₁₋₆ alkoxy, haloC₁₋₆ alkyl, haloC₁₋₆ alkoxy, cyano, nitro, —OC(O)C₁₋₆ alkyl, —NH₂, —NHC₁₋₆ alkyl, —NHC(O)C₁₋₆ alkyl and —N(C₁₋₆ alkyl)(C₁₋₆ alkyl)),
—C(O)NHalkyl (e.g. C(O)NHC₁₋₆ alkyl such as methyl ester, ethyl ester, propyl ester, butyl amide), —C(O)NHdialkyl (e.g. C(O)NH(C₁₋₆ alkyl)₂),
aminoalkyl (e.g., HNC₁₋₆ alkyl-, C₁₋₆ alkylHN-C₁₋₆ alkyl- and (C₁₋₆ alkyl)₂N—C₁₋₆ alkyl-), thioalkyl (e.g., HSC₁₋₆ alkyl-),
carboxyalkyl (e.g., HO₂CC₁₋₆ alkyl-),
carboxyesteralkyl (e.g., C₁₋₆ alkylO₂CC₁₋₆ alkyl-),
amidoalkyl (e.g., H₂N(O)CC₁₋₆ alkyl-, H(C₁₋₆ alkyl)N(O)CC₁₋₆ alkyl-),
formylalkyl (e.g., H(O)CC₁₋₆ alkyl-),
acylalkyl (e.g., C₁₋₆ alkyl(O)CC₁₋₆ alkyl-),
nitroalkyl (e.g., O₂NC₁₋₆ alkyl-),
replacement of CH₂ with C═O, and
where 2 carbon atoms (1,2 or 1,3) are substituted by one end each of a —O—(CH₂)ₙ—O— or —NH—(CH₂)ₙ—NH— group, wherein n is 1 or 2.

In some embodiments, each R^{a} is independently selected from H, chloro, fluoro, bromo, iodo, haloC₁₋₆ alkyl, (e.g. fluoroC₁₋₆ alkyl, such as —CHF₂ and —CF₃), C₁₋₆ alkyl, C₁₋₆ alkoxy C₃₋₆cycloalkyl, aziridinyl, azetidinyl, pyrrolidinyl, piperidinyl, haloaziridinyl, haloazetidinyl, halopyrrolidinyl, halopiperidinlyl and haloC₁₋₆ alkoxy (e.g. fluoroC₁₋₆ alkoxy, such as —OCHF₂ and —OCF₃). In further embodiments, each R^{a} is independently selected from H, chloro, fluoro, bromo, iodo, haloC₁₋₃ alkyl, C₁₋₃ alkyl, C₁₋₃ alkoxy, C₃₋₆cycloalkyl and halo C₁₋₃ alkoxy.

In some embodiments, Each of A¹-A⁵ is C—R^{a}. In still further embodiments, A³ is not C—H. In some further embodiments thereof one or two of C—R^{a} is C—R^{aa}, wherein R^{aa} is not H, with the remainder C—H. In still further embodiments, R^{aa} is selected from F, Cl, I, Me, cyclopropyl, difluoroazetidinyl, OMe, CHF₂, CF₃OCHF₂ and OCF₃

In some embodiments, at least A³ is C—R^{aa}, and in a further embodiments thereof, A³ is not C—H. In some further embodiments A³ is C—R^{aa} and A¹, A², A⁴ and A⁵ are each C—H. In some embodiments A³ is C—R^{aa} and one of A⁴ or A² is the same or different C—R^{aa}. In still further embodiments, each R^{aa} is independently selected from F, Cl, I, Me, cyclopropyl, difluoroazetidinyl, OMe, CHF₂, CF₃ OCHF₂ and OCF₃.

In some embodiments, any one or two of A¹, A², A³, A⁴ or A⁵ may be N. In some embodiments, A⁵ or A¹ is N; or A² or A⁴ is N; or A¹ and A⁵ are both N; or A² and A⁴ are both N; or A¹ and A⁴, or A² and A⁵ are both N; or A¹ and A² or A⁴ and A⁵ are both N. In some of these embodiments A³ is C—R^{a}. In still further embodiments, R^{aa} is selected from F, Cl, I, Me, cyclopropyl, difluoroazetidinyl, OMe, CHF₂, CF₃ OCHF₂ and OCF₃

In some embodiments Q is a 5-membered heteroaromatic ring having 2, 3 or 4 ring heteroatoms, at least one of which must be N and the remaining heteroatoms independently selected from N, O and S, and is selected from heterocyclic formulas (a)-(kk), which may optionally substituted with a group Q^{a} where permissible (where the bonds labelled # are attached to NH and the bonds labelled * are attached to the aryl ring defined by A¹-A⁵):

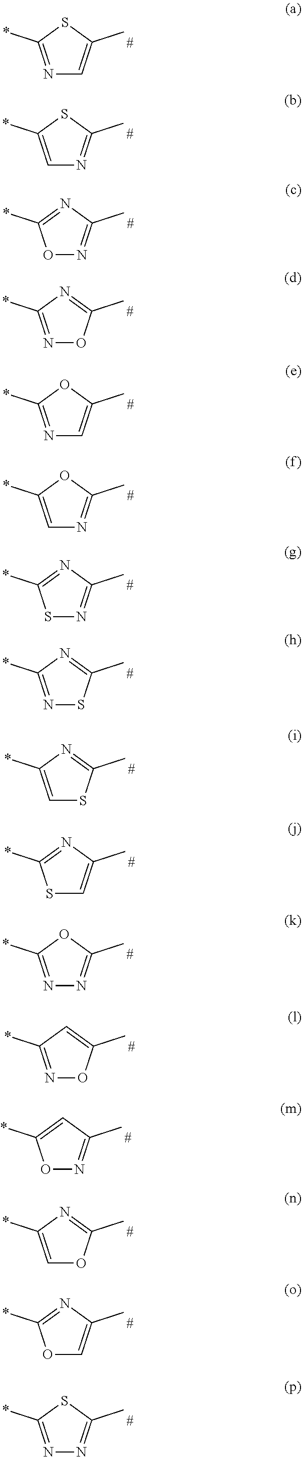

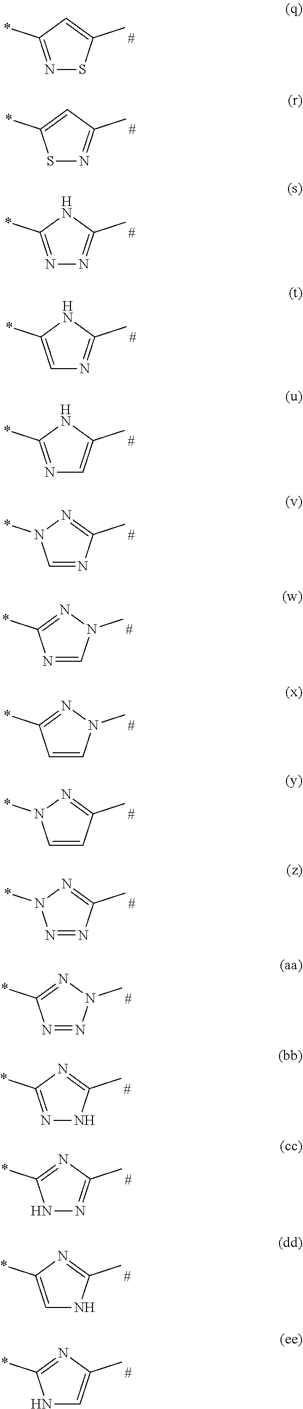

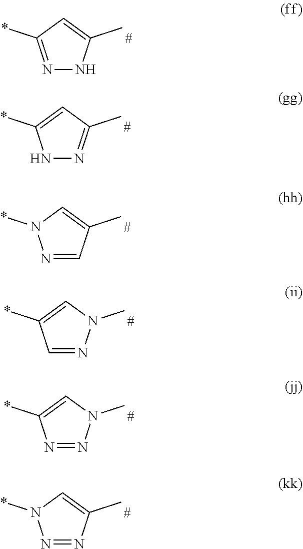

Where Q contains a carbon or nitrogen ring atom bearing a hydrogen atom (e.g. (a), (b), (e), (f), (i). (j), (l), (m), (n), (o), (q), (r), (t), (u), (v), (w), x), (y), (bb), (cc), (dd), (ee), (ff), (gg), (hh) and (ii)), that carbon or nitrogen atom may be optionally substituted (i.e. the hydrogen atom replaced) with a group Q^{a}, selected from halo, haloalkyl and alkyl. For example, Q^{a} may be selected from, Cl, F, Br, I, C₁₋₆ alkyl (e.g. methyl, ethyl, n- and i-propyl, n-, sec- and t-butyl, pentyl, hexyl), and haloC₁₋₆ alkyl (e.g. (CH₂)_{q}CF₃, (CH₂)_{q}CCl₃, (CH₂)_{q}CBr₃, (CH₂)_{q}CHF₂, (CH₂)_{q}CHCl₂, and (CH₂)_{q}CHBr₂, (CH₂)_{q}CH₂F, (CH₂)_{q}CH₂Cl, and (CH₂)_{q}CH₂Br, where q is 0, 1, 2, 3, 4 or 5). In further embodiments, Q^{a} is selected from Cl, F, Br, I, CH₃, CF₃, CBr₃, and CCl₃. In some embodiments said carbon or nitrogen ring atom is unsubstituted. In other embodiments said carbon or nitrogen ring atom is substituted with Q^{a}.

Some further non-limiting examples of Q having a carbon or nitrogen ring atom substituted with Q^{a} include:

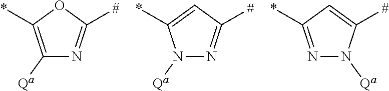

Further embodiments include where Q^{a}, is for example Cl, F, Br, I, C₁₋₆ alkyl (e.g. methyl, ethyl, n- and i-propyl). In some further embodiments Q^{a} is methyl.

In some embodiments, Q is a 5-membered heteroaromatic ring having 2 or 3 ring heteroatoms, at least one of which must be N and the remaining independently selected from N, O and S, where a carbon or nitrogen ring atom may be optionally substituted as described above.

In some embodiments, Q is selected from one or more of Q₁, Q₂, Q₃, Q₄ or Q₅:

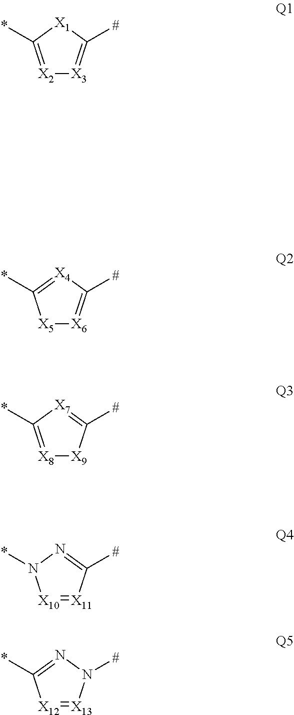

wherein
X₁ is O, S or NH and X₂ and X₃ are independently CH or N, provided both are not CH (formulae (a), (b), (e), (f), (k), (p), (s), (t) and (u));
X₅ is O, S or NH and X₄ and X₆ are independently CH or N, provided both are not CH (formulae (c), (g), (j), (f), (m), (o), (r), (cc), (ee) and (gg));
X₉ is O, S or NH and X₇ and X₈ are independently CH or N, provided both are not CH (formulae (d), (h), (i), (1), (n), (q), (bb), (dd) and (ff)); and
X₁₀—X₁₃ are in dependently CH or N (formulae (v), (w), (x) and (y));
and wherein Q may be unsubstituted, or substituted with Q^{a}.

In some embodiments, Q has 3 ring heteroatoms (formulae (c), (d), (g), (h), (k) (p), (s), (v), w), (jj) and (kk)). In some examples thereof, Q has two ring nitrogen atoms and one ring oxygen atom. In other examples thereof, Q has two ring nitrogen atoms and one ring sulfur atom. In other examples thereof, Q has three ring nitrogen atoms.

In other embodiments Q has 2 ring heteroatoms (formulae (a), (b), (e), (f), (i) (j) (l), (m), (n), (o), (q), (r), (t), (u), (x), (y), (hh) and (ii)). In some examples thereof, Q has one ring nitrogen atom and one ring oxygen atom. In other examples thereof, Q has one ring nitrogen atom and one ring sulfur atom. In other examples thereof, Q has two ring nitrogen atoms.

In other embodiments, Q has one or two nitrogen ring atoms and one ring oxygen atom (formulae (c), (d), (e), (f), (k) (l) (m), (n) and (o)).

In other embodiments Q has one or two nitrogen ring atoms and one ring sulfur atom (formulae (a), (b), (g), (h), (i) (j) (p), (q) and (r)).

In other embodiments, Q has two or three or 4 nitrogen ring atoms and no O or S ring atoms (formulae (s), (t), (u), (v), (w), (x), (y), (z), (aa), (bb), (cc), (dd), (ee), (ff), (gg) (hh), (ii), (jj) and (kk).

In some embodiments, Q is selected from (c), (d), (f), (g), (h), (i), (j), (k) and (p).

In some embodiments, Q is selected from (c), (d), (f), (h), (i), (j), (k) and (p).

In some embodiments, Q is selected from (c), (d), (f), (i), (j), (k) and (p).

In some embodiments, Q is an oxadiazolyl group (formulae (c), (d) and (k)).

In some embodiments, Q is selected from (d), (f), (i), (k), (l), (n), (v), (y), (ee), (ff) (hh) and (kk).

In some further embodiments, Q is selected from (f) and (k).

In some embodiments, including any one embodiment of Q as described above, such as where Q is selected from (f), (i), (k) (n) and (v), and including where a ring carbon or nitrogen atom may be optionally substituted with Q^{a}, W contains 1 or 2 nitrogen ring atoms. In some embodiments, W contains at least 1 nitrogen ring atom at a position ortho- to C—R^{b}, i.e. having the formula (A), (B), (C), (D), (F), (G), or (J). In further examples of any one such embodiments, R^{d} is H, OH, C, Br, F, I, CH₃ or OCH₃. In some further embodiments, W is selected from:

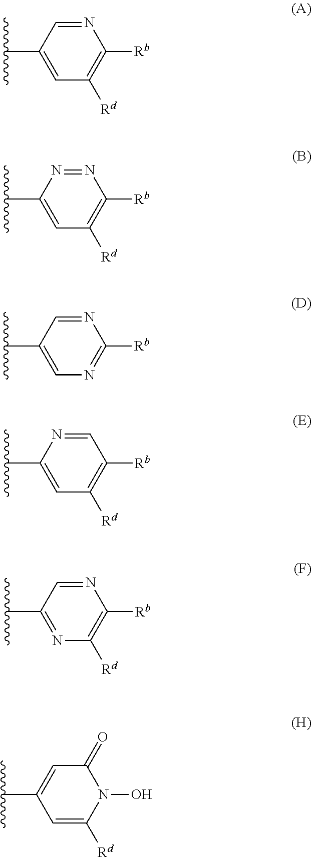

and R^{d} is H, OH, F, I, Cl, Br, CH₃ or OCH₃. R^{b} may K—NR^{C}—Y, or a tautomer thereof.

In some embodiments, including any one embodiment of Q as described above, such as where Q is selected from (f), (i), (k) (n) and (v), and including where a ring carbon or nitrogen atom is optionally substituted with Q^{a}, W is a 6-membered N-containing heterocycle (aromatic or non-aromatic) selected from:

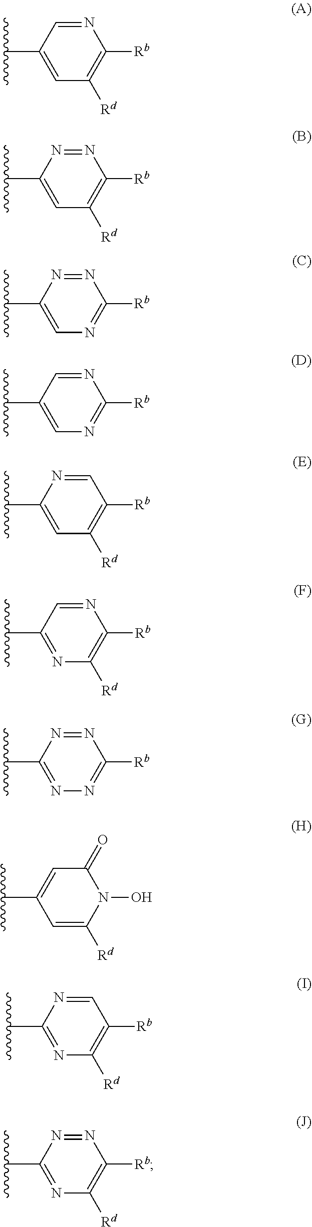

wherein
R^{d} is H, OH, F, CL, I, Br, CH₃ or OCH₃; and R^{b} is —K—NR^{c}—Y, or a tautomer thereof.

In some embodiments, including any one of the embodiments described above, R^{b} is —K—NR^{c}—Y, or tautomer thereof, where:
K is SO₂, C(═O), C(═NH), or NHC(═O),
R^{c} is H, C₁₋₆ alkyl (e.g. C₁₋₃ alkyl, such as CH₃, CH₂CH₃ or (CH₂)₂CH₃); or hydroxyC₁₋₆ alkyl (e.g. hydroxyC₁₋₃ alkyl; such as —(CH₂)OH, —(CH₂)₂OH, —CH(OH)CH₂OH, CH(OH)CH₃, —(CH₂)₃OH, —CH(OH)(CH₂)₂OH, —CH₂CH(OH)CH₂OH, —(CH(OH))₂CH₃ and —(CH(OH))₂CH₂OH); and
Y is OH, NH₂, NHC₁₋₆ alkyl, NHC(═O)H, NH(C(═O)C₁₋₆ alkyl, hydroxyC₁₋₆ alkyl (e.g. hydroxyC₁₋₃ alkyl; such as —(CH₂)OH, —(CH₂)₂OH, —CH(OH)CH₂OH, CH(OH)CH₃, —(CH₂)₃OH, —CH(OH)(CH₂)₂OH, —CH₂CH(OH)CH₂OH, —(CH(OH))₂CH₃ and —(CH(OH))₂CH₂OH); or (SC₁₋₆ alkyl)C₁₋₆ alkyl (e.g. (SC₁₋₃ alkyl)C₁₋₃ alkyl, such as —(CH₂)SCH₃, —(CH₂)₂SCH₃, —CH(SCH₃)CH₂SCH₃, CH(SCH₃)CH₃, —(CH₂)₃SCH₃, —CH(SCH₃)(CH₂)₂SCH₃, —CH₂CH(SCH₃)CH₂SCH₃, —(CH(SCH₃))₂CH₃ and —(CH(SCH₃))₂CH₂SCH₃).

In some embodiments R^{b} is

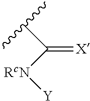

wherein
X′ is O or NH,
R^{c} is H or C₁₋₆ alkyl; or hydroxyC₁₋₆ alkyl; and
Y is OH or NH₂.
or W is of formula (H).

In still further embodiments, R^{b} is selected from:

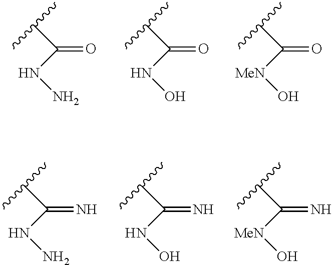 [SMILES: *C(=O)NN ; *C(=O)NO ; *C(=O)N(C)O ; *C(=N)NN ; *C(=N)NO ; *C(=N)N(C)O]

or a tautomer thereof. By way of example, in some further embodiments, R^{b} may be either

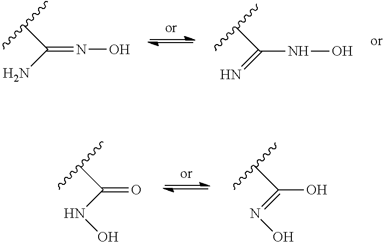 [SMILES: *C(N)=NO ; *C(=N)NO ; *C(=O)NO ; */C(O)=N/O]

The amidine group may be present as a substantially pure (e.g. >90%, or 95% or 99%) E- or Z-isomer, or may be a mixture of E- and Z-isomers.

In some further embodiments, Q is of formula (f) or (k), and R^{b} is —C(═X′)—NR^{c}—Y, wherein X′ is O or NH, R^{c} is H or Me, and Y is OH or NH₂.

In some embodiments, W comprises the moiety C(═O)—N—OH, e.g. W has the formula (H) or where R^{b} is C(═O)—NR′—OH, or NHC(═O)—NR′—OH (where R′ is H, C₁₋₆ alkyl or hydroxyC₁₋₆ alkyl). In still further embodiments, R^{d} is H, OH, F, CL, I, Br, CH₃ or OCH₃.

In some embodiments, R^{b} is OH and W is (E) or (I). R^{d} may be selected from H, OH, F, Cl, Br, I, C₁ alkyl, C₂ alkyl, C₃ alkyl, C₄ alkyl, C₅ alkyl, C₆ alkyl, C₁ alkoxy, C₂ alkoxy, C₃ alkoxy, C₄ alkoxy C₅ alkoxy, C₆ alkoxy. In some further embodiments, R^{d} is H, OH, F, Cl, Br, I, CH₃ or OCH₃.

In some embodiments, R^{b} is K—NR^{c}—Y, or a tautomer thereof, or —(CH₂)ₚ—NH—OH. In further embodiments, NHR^{e} is NH₂, NHC₁₋₃ alkyl, NHC(═O)H, NH(C(═O)C₁₋₃ alkyl. R^{d} may be selected from H, OH, F, Cl, Br, I, C₁ alkyl, C₂ alkyl, C₃ alkyl, C₄ alkyl, C₅ alkyl, C₆ alkyl, C₁ alkoxy, C₂ alkoxy, C₃ alkoxy, C₄ alkoxy C₅ alkoxy, C₆ alkoxy. In some further embodiments, R^{d} is H, OH, F, Cl, Br, I, CH₃ or OCH₃.

In some embodiments, R^{b} is K—NR^{c}—Y, or a tautomer thereof, or —(CH₂)ₚ—NH—OH, and W is one of (A), (B), (C), (D), (F), (G), (H), (J). In further embodiments, NHR^{e} is NH₂, NHC₁₋₃ alkyl, NHC(═O)H, NH(C(═O)C₁₋₃ alkyl. R^{d} may be selected from H, OH, F, Cl, Br, I, C₁ alkyl, C₂ alkyl, C₃ alkyl, C₄ alkyl, C₅ alkyl, C₆ alkyl, C₁ alkoxy, C₂ alkoxy, C₃ alkoxy, C₄ alkoxy C₅ alkoxy, C₆ alkoxy. In some further embodiments, R^{d} is H, OH, F, Cl, Br, I, CH₃ or OCH₃.

In some embodiments, including any embodiment of Q as described above, and including where a ring carbon or nitrogen atom is optionally substituted with Q^{a}, W is (E) or (I) and R^{b} is OH. In still further embodiments thereof, R^{d} is H, OH, F, Cl, I, Br, CH₃ or OCH₃.

In some embodiments, when W is of formula (I), then R^{b} is not —(C═O)—NH—OH.

The disclosure also provides compounds of Formula (I″), wherein R^{b} and R^{d} of Formula (I′) are transposed, and pharmaceutically acceptable salts and solvates thereof.

In another aspect, there is provided compounds of Formula (IA), comprising compounds of Formula (I′), and pharmaceutically acceptable salts and solvates thereof and compounds of Formula (I″), wherein R^{b} and R^{d} of Formula (I′) are transposed, and pharmaceutically acceptable salts and solvates thereof. Any one of the embodiments described for (I′) apply to (II′). In some embodiments W does not contain a N ring atom adjacent, or ortho-, to a ring carbon bearing a OH group.

For compounds that are capable of tautomerism, under certain conditions, e.g., solvents, salt forms, pH, etc, one tautomer may be a preferred form over another, but that changes in conditions may result in formation of the other tautomer. It will be understood that, where appropriate, tautomers of Formula (I′), for example certain R^{b} groups, may exist, and are also encompassed by the disclosure herein. Unless otherwise specified, a compound depicted in one tautomeric form is also a disclosure of the other tautomeric form. Some exemplary tautomeric R^{b} groups include:

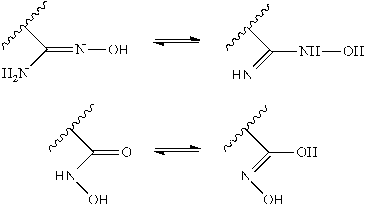 [SMILES: *C(N)=NO ; *C(=N)NO ; *C(=O)NO ; */C(O)=N/O]

It will be appreciated that tautomers can exists in E- and Z-forms where appropriate, either as substantially pure (e.g. >90%, or 95% or 99%) E- or Z-isomers or a mixture of isomers.

In some embodiments, compounds disclosed herein have any one or more of any of A¹-A⁵, R^{a}, R^{aa}, Q, W, R^{b}, R^{c}, R^{d} and R^{e} as depicted in the compounds disclosed or described in the Examples 1-120. Thus, also expressly disclosed herein are compounds having any combination of, for example, A¹-A⁵ and Q, A¹-A⁴ and W, Q and W, R^{b} and W, as depicted in any one of compounds 1-120.

It will also be recognised that certain compounds of the disclosure may possess asymmetric centres and are therefore capable of existing in more than one stereoisomeric form, such as enantiomers and diastereomers. The invention thus also relates to optically active compounds and compounds in substantially pure isomeric form at one or more asymmetric centres, e.g., enantiomers having greater than about 90% ee, such as about 95% or 97% ee or greater than 99% ee, as well as mixtures, including racemic mixtures, thereof. Such isomers may be prepared by asymmetric synthesis, for example using chiral intermediates or reagents, enzymes, or mixtures may be resolved by conventional methods, e.g., chromatography, recrystallization, or use of a resolving agent.

In some embodiments, at least one of R^{a}, R^{b}, R^{c} R^{d} and R^{e} possess at least one chiral centre. In further such embodiments, at one of R^{a}, R^{b} and R^{c}, R^{d} and R^{e} possess a chiral centre. In some embodiments, one of R^{a}, R^{b}, R^{c}, R^{d} or R^{e} has one chiral centre and the compound exists as a mixture of enantiomers, e.g. a racemic mixture, or the compound may be substantially enantiomerically pure, i.e. substantially the R- or S-form. One example is R^{b}═—CH₂C*H(OH)CH₂OH, where C* is the chiral centre. Compounds bearing R^{b}═—CH₂C*H(OH)CH₂OH, may exist as a mixture of enantiomers (e.g. a racemic mixture) or may be in the substantially enantiomerically pure R- or S-form.

Compounds of the disclosure may be prepared using any suitable methodology. The Examples section sets out numerous methodologies that can be further extrapolated to the preparation the compounds of the disclosure, using routine skill and knowledge, such as by varying starting materials and reagents, solvents, etc. and methods for the preparation of heterocycles as known in the art (see for example, Aurelio, L., et al, J. Med. Chem., 2016, 59, 965-984; Sharma, S., Sulfur Reports, 1989, 8, 327-469). In some non-limiting embodiments, compounds can be prepared by coupling a precursor comprising the R^{a}-substituted phenyl and Q moieties (or their precursor(s)) with an appropriate W moiety or precursor thereof. In other non-limiting embodiments compounds may be prepared by internal cyclization (to produce a Q′ moiety), of a precursor compound comprising the R^{a}-substituted phenyl and W moieties, (or precursor(s) thereof). As used herein, a “precursor” includes a chemical entity or moiety that may be converted to the desired compound or moiety by one or more chemical transformations and/or couplings. Some illustrative generalized schemes for preparation of various compounds of formula (I′) and/or precursors therefor, are set out in Schemes A-V below.

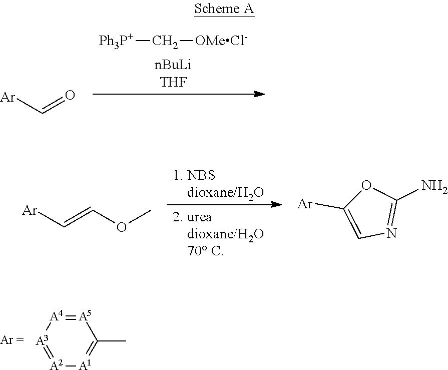

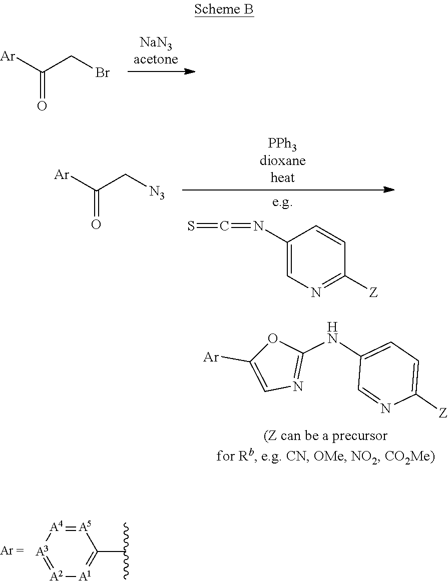

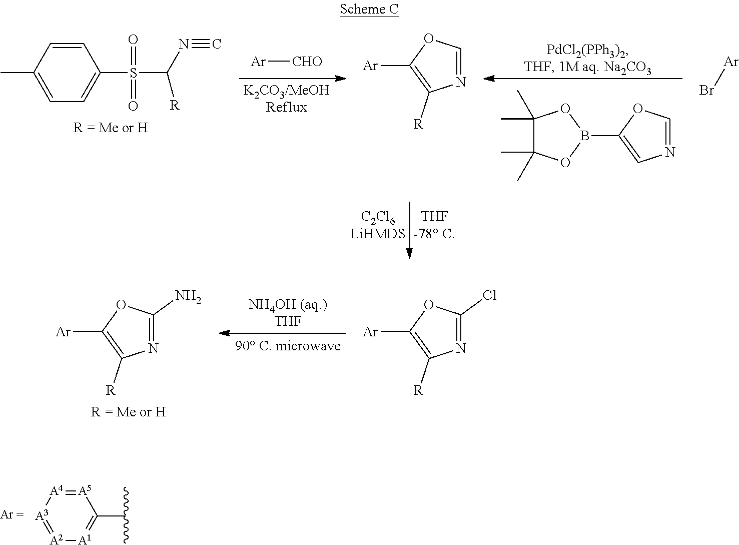

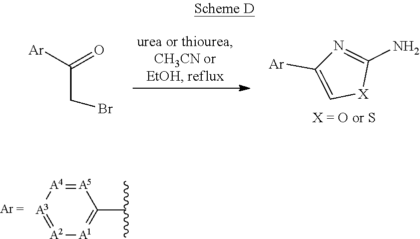

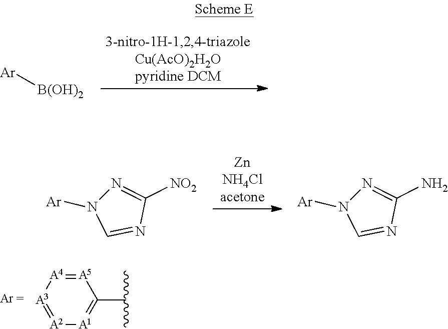

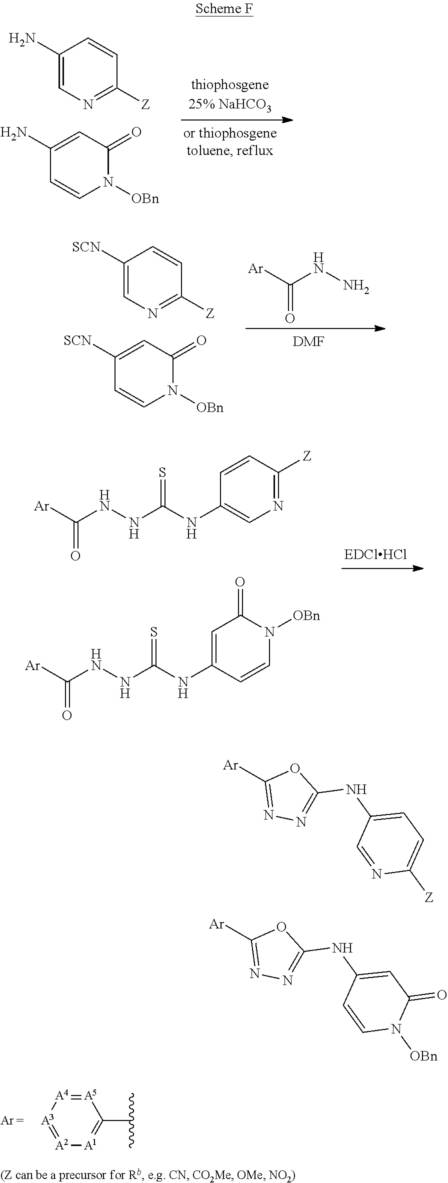

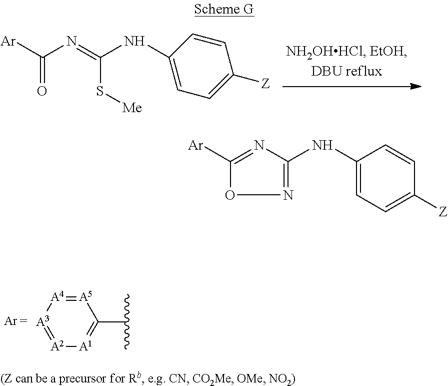

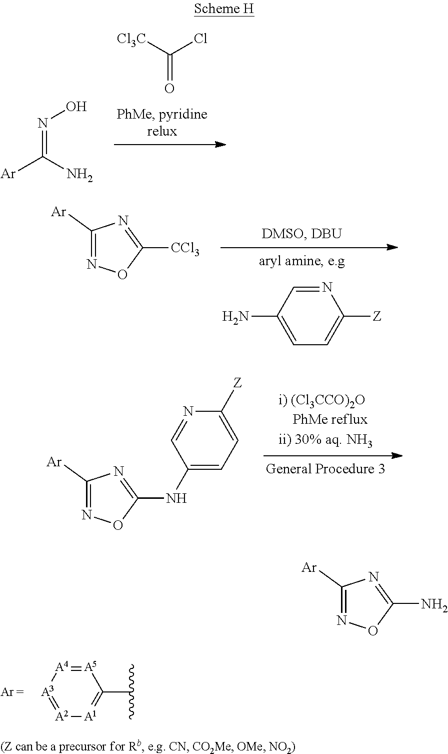

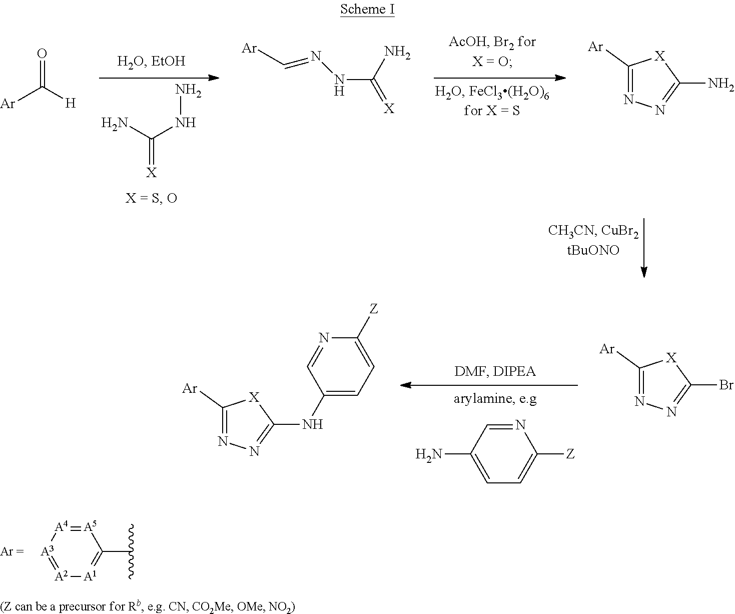

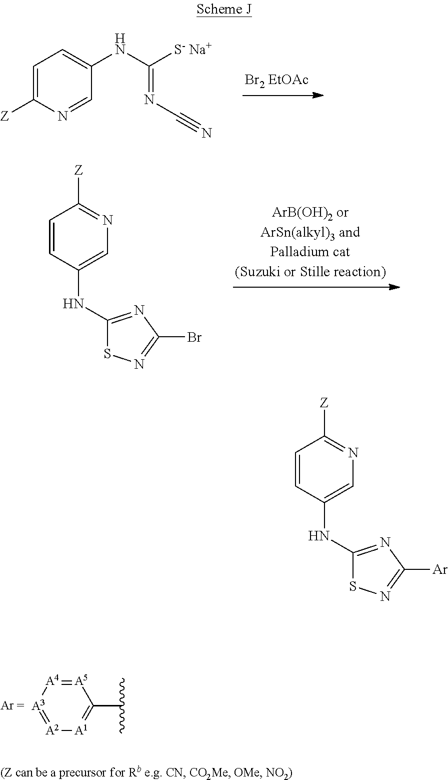

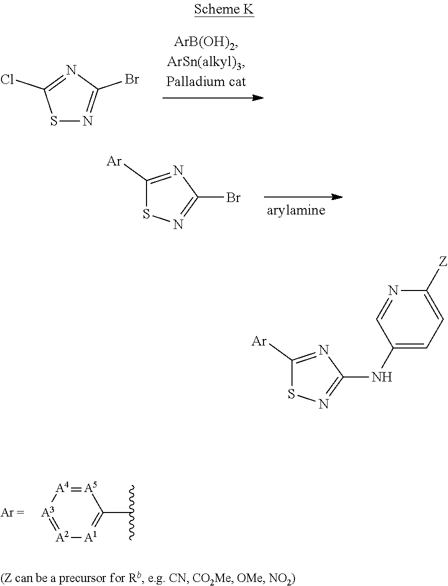

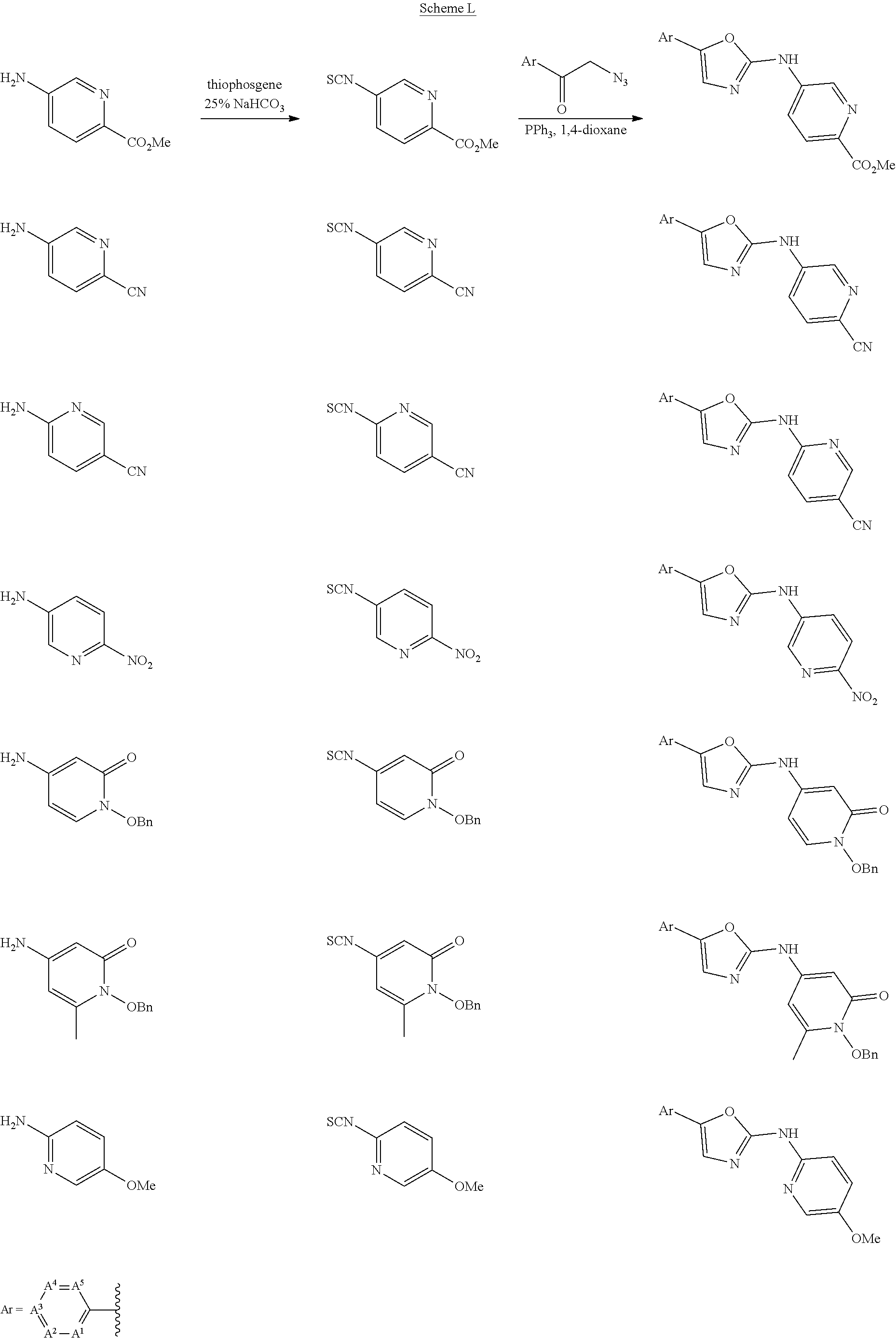

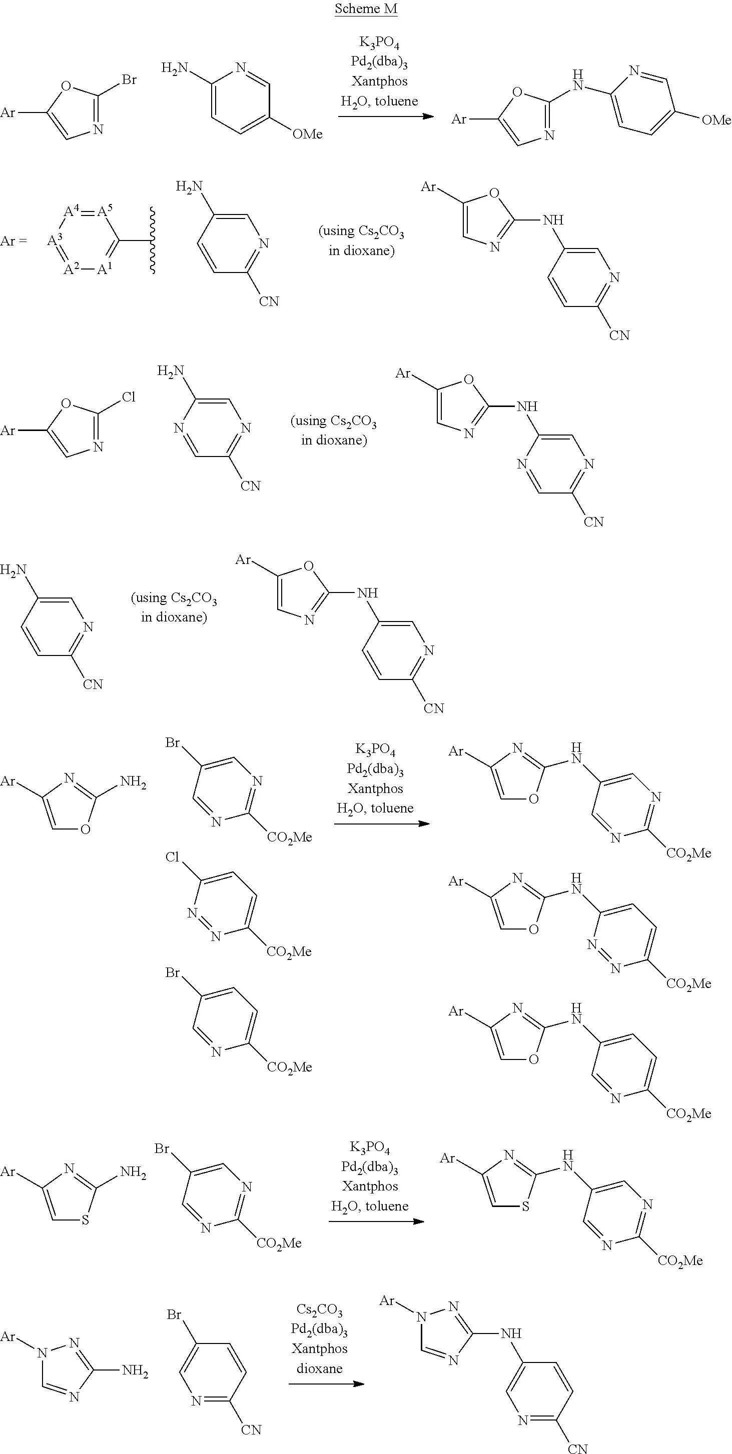

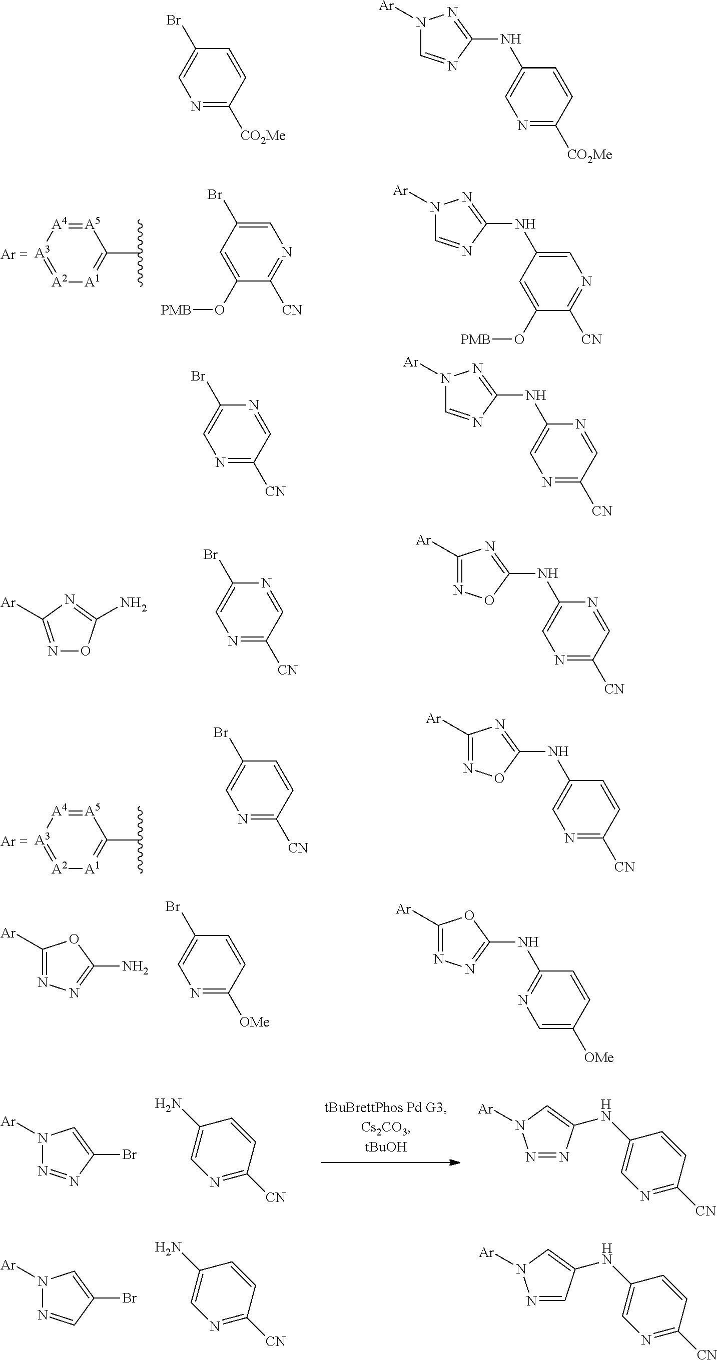

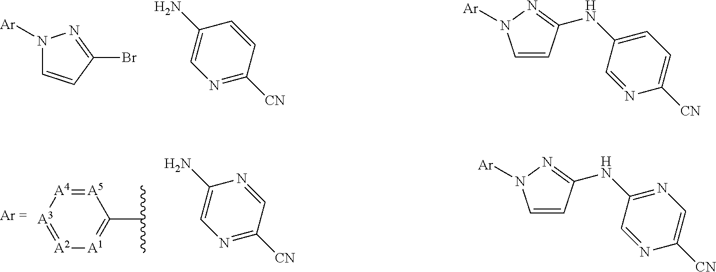

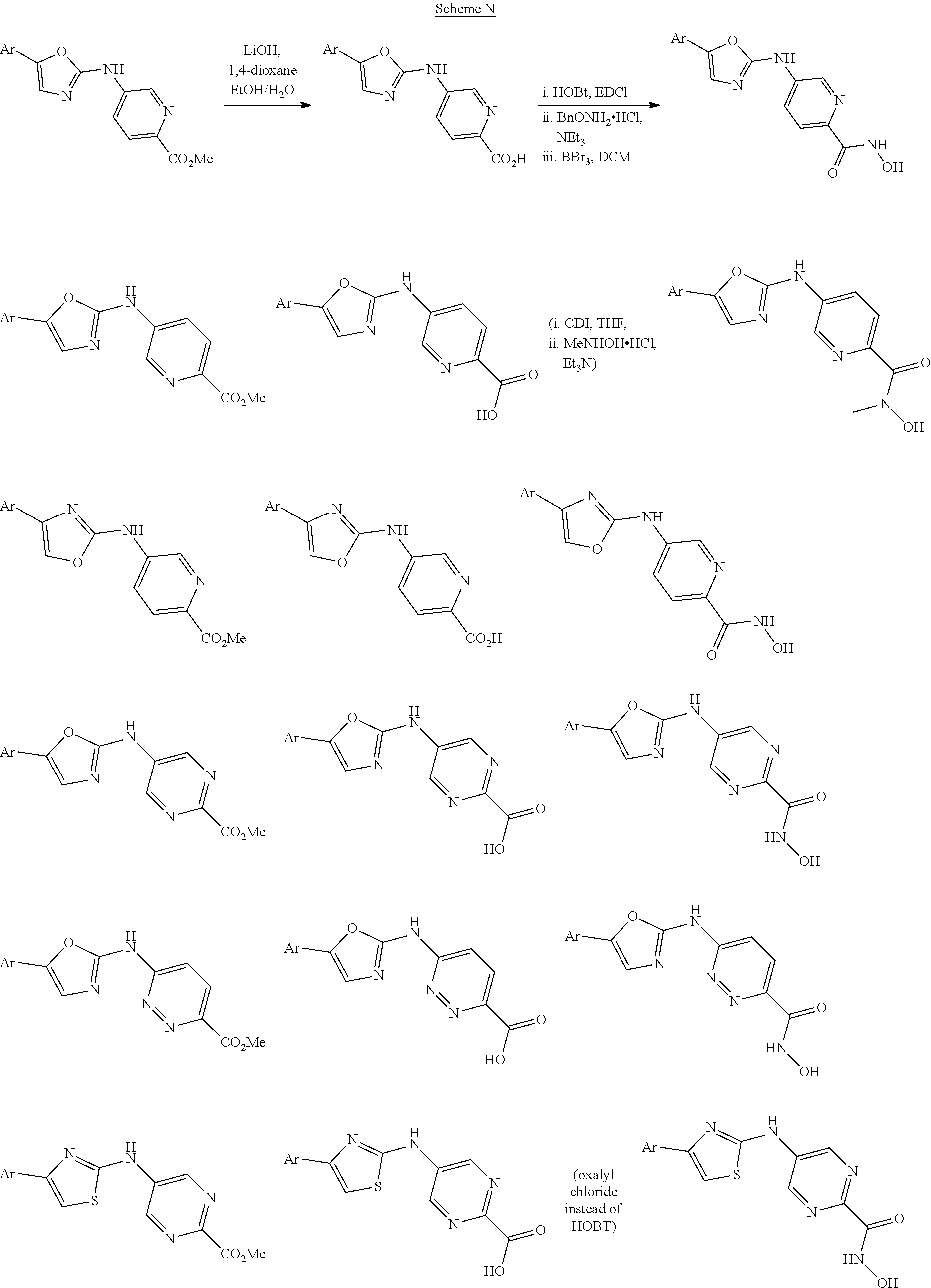

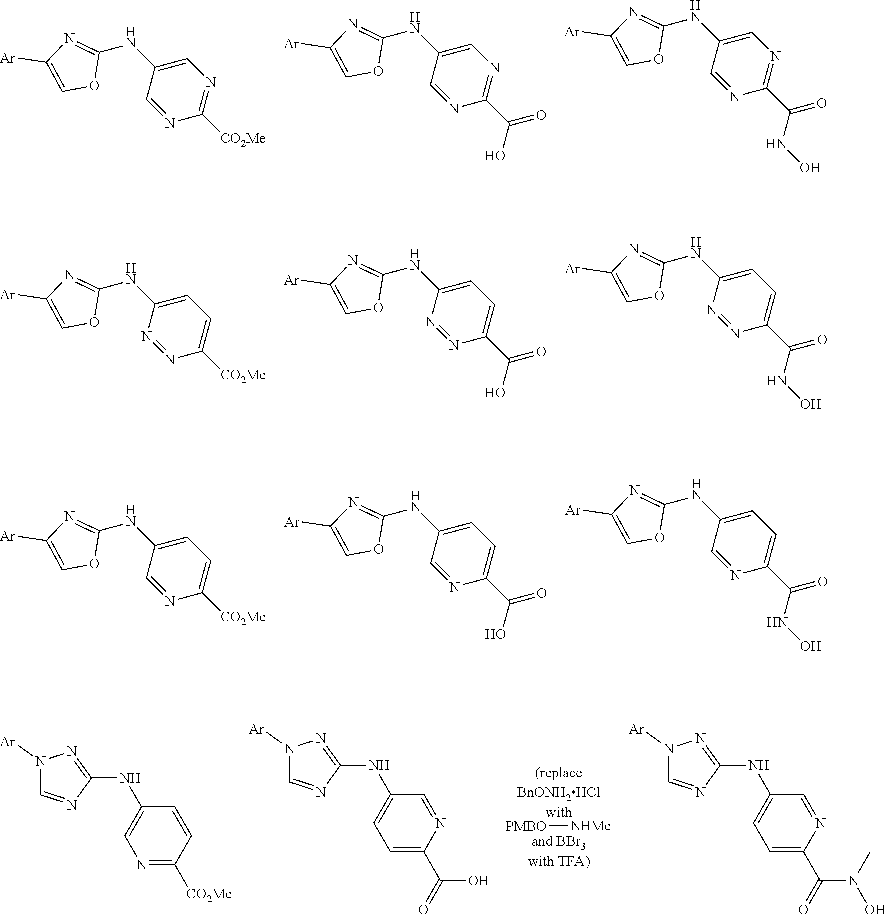

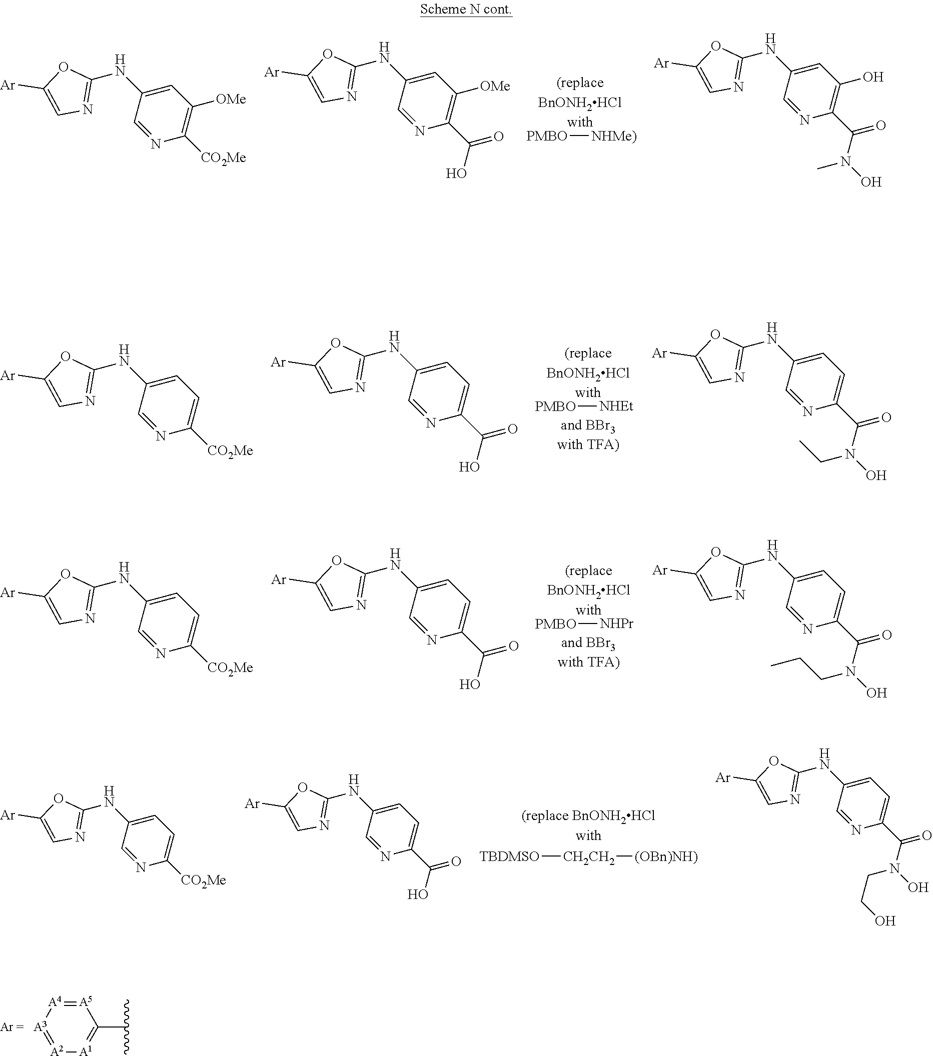

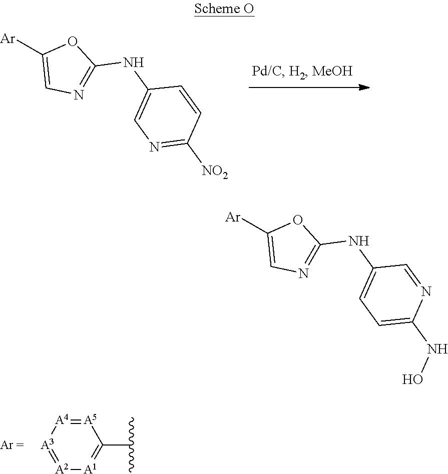

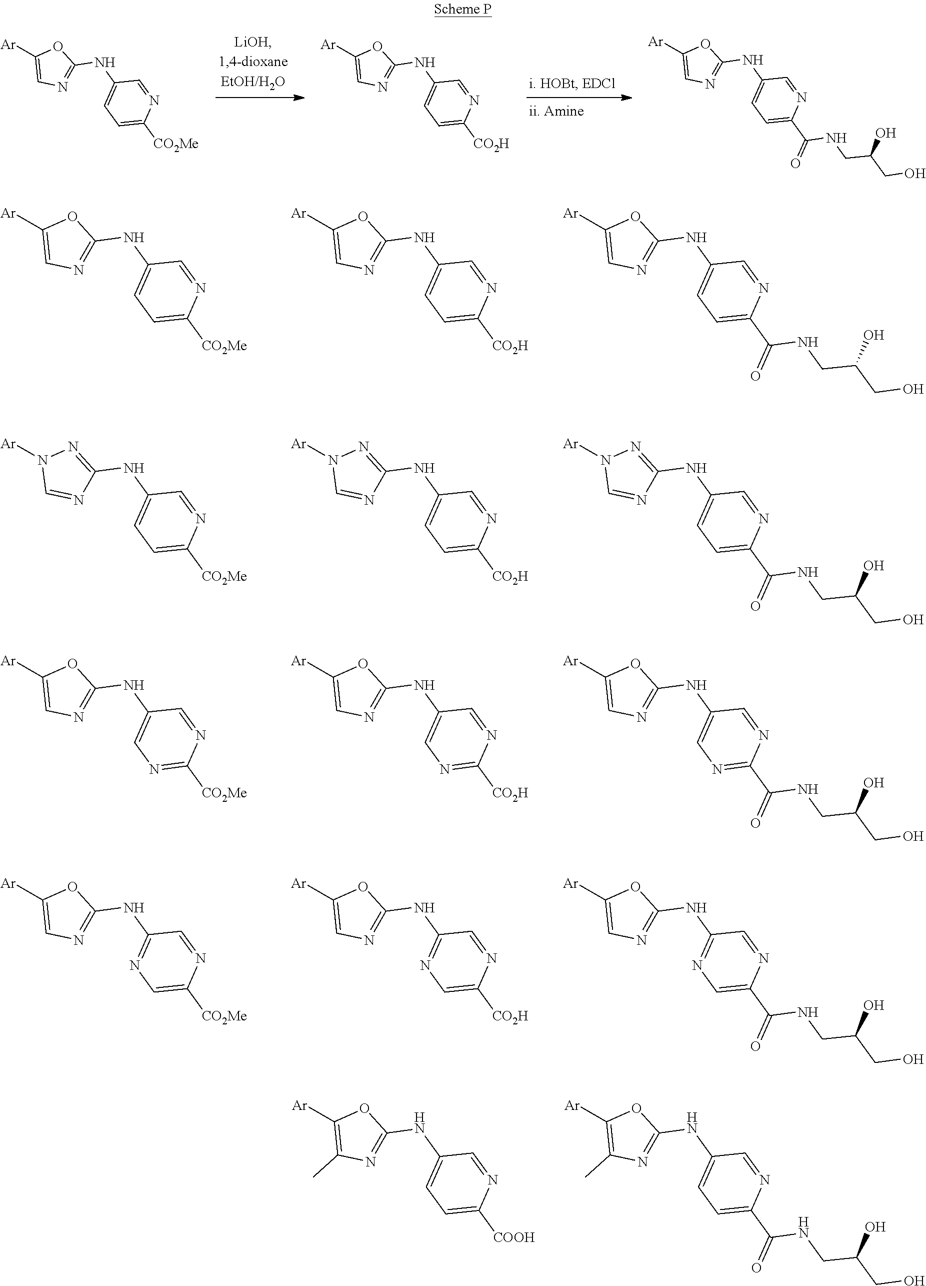

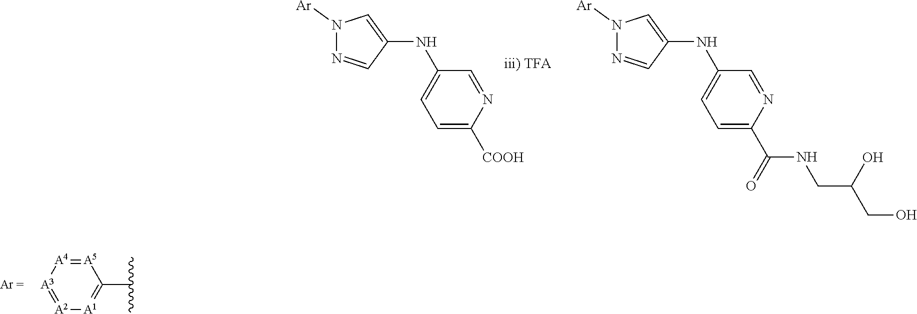

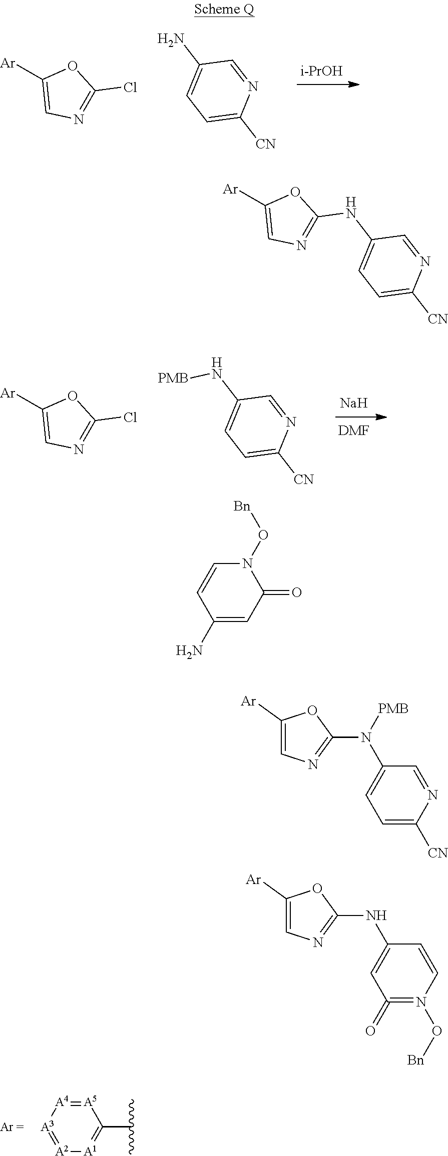

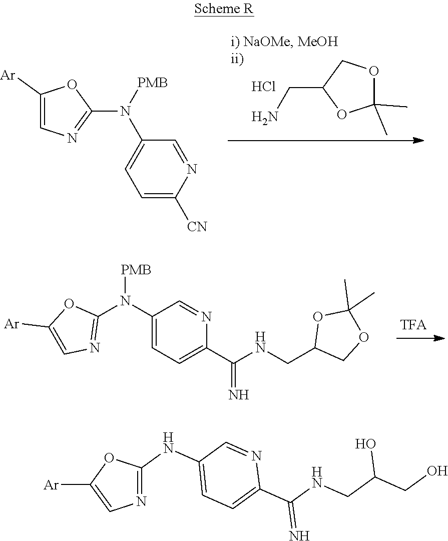

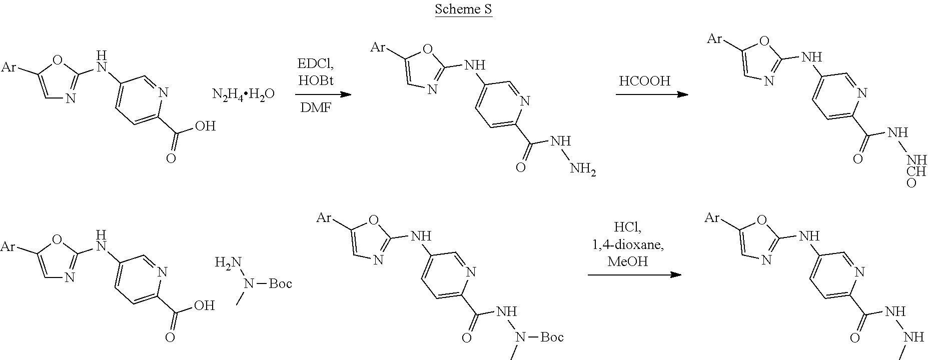

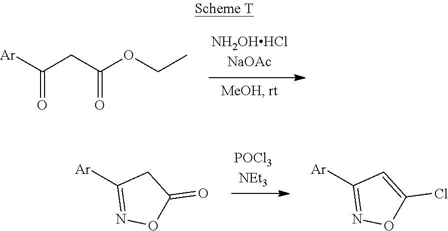

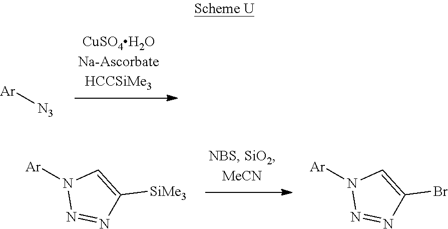

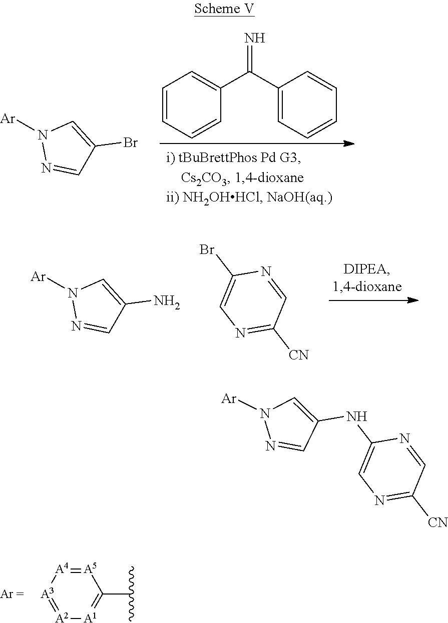

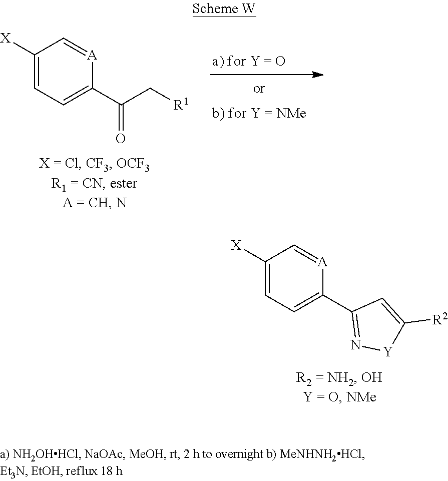

It will be recognised that during the processes for the preparation of compounds of the disclosure, it may be necessary or desirable to protect certain functional groups which may be reactive or sensitive to the reaction or transformation conditions undertaken. Examples of such groups include: OH (including diols), NH₂, CO₂H, SH and C═O. Suitable protecting groups for such functional groups are known in the art and may be used in accordance with standard practice. As used herein, the term “protecting group”, refers to an introduced functionality which temporarily renders a particular functional group inactive under certain conditions. Such protecting groups and methods for their installation and subsequent removal at an appropriate stage are described in Protective Groups in Organic Chemistry, 3^{rd} Edition, T. W. Greene and P. G. Wutz, John Wiley and Sons, 1999, the entire contents of which are incorporated herein by reference. Exemplary forms of protected groups include:
for amino (NH₂)-carbamates (such as Cbz, Boc, Fmoc), benzylamines, acetamides (e.g. acetamide, trifluoroacetamide);
for carbonyl—acetals, ketals, dioxanes, dithianes, and hydrazones;
for hydroxy—ethers (e.g. alkyl ethers, alkoxylalkyl ethers, allyl ethers, silyl ethers, benzyl ethers, such as p-methoxybenzyl, tetrahydropyranyl ethers), carboxylic acid esters, acetals (e.g. acetonide and benzylidene acetal);
for thio (SH)-ethers (e.g. alkyl ethers, benzyl ethers), esters; and
for CO₂H—esters (e.g. alkyl esters, benzyl esters).

Further General Procedures for the preparation of compounds of the disclosure and/or precursors therefor, are set out below

### General Procedure 1: General Procedure for Amidoxime (Scheme GP 1)

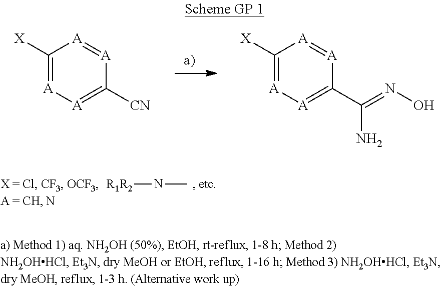

Method 1: NH₂OH (aq. 50%, 1.05 equiv.) was added dropwise to a solution of nitrile (1.0 equiv.) in EtOH at rt and the mixture was stirred at this temperature for 1 h or refluxed for 8 h. The solvent was removed in vacuo thus giving the titled amidoxime as a white solid with quantitative yield.

Method 2: NH₂OH·HCl (3-8 eq.) and Et₃N (3-8 eq.) were added to a suspension of nitrile in dry MeOH or EtOH. The mixture was stirred at reflux for 1-16 h. LCMS indicated that the reaction was complete. The volatiles were removed and the precipitated solids were suspended in water, collected by filtration then washed well with H₂O. The solids were washed with Et₂O and DCM and dried to provide the title compound. If required, compounds can be further purified via preparative HPLC.

Method 3: NH₂OH·HCl (3-8 eq.) and Et₃N (3-8 eq.) were added to a suspension of nitrile in dry MeOH. The mixture was stirred at reflux for 1-3 h. LCMS or TLC indicated that the reaction was complete. The reaction was cooled to room temperature and the precipitated solids were collected by filtration then washed well with H₂O. If no solid formed at rt, the reaction was diluted with water (at least 3 fold, MeOH volume) prior to filtration to induce precipitation. The solids were washed with Et₂O, and as indicated with CH₂Cl₂, then dried to provide the title compound. If required, compounds can be further purified via preparative HPLC.

### General Procedure 2: Nucleophilic Substitution (Scheme GP 2)

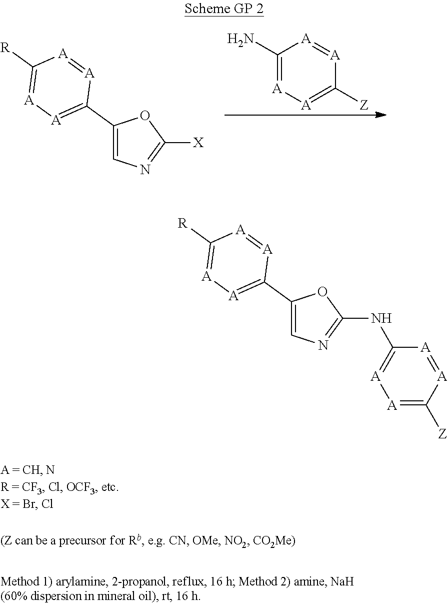

Method 1: A mixture of oxazole chloride or bromide (1.0 eq.) and aromatic amine or heteroaromatic amine (1.5-2.0 eq.) in anhydrous 2-propanol was stirred at reflux for 16 h. Upon cooling, the reaction mixture was concentrated in vacuo. The resultant crude was suspended in H₂O, filtered and washed with Et₂O. If required, the filtered solids were purified through reverse-phase chromatography (H₂O, MeCN 10-100%) to yield the desired product.

Method 2: NaH (1.5-3.0 equiv., 60% dispersion in mineral oil) was added to a solution of aromatic amine or heteroaromatic amine (1.5-2.0 equiv.) in DMF at 0° C. under an atmosphere of nitrogen. The mixture was stirred for 0.5 h at this temperature. A solution of chloride (1.0 equiv.) in DMF was added dropwise to the mixture. After a further 16 h, at rt, the solid was collected by filtration, washed with water, DCM, Et₂O and dried under vacuum. If required, the filtered solids were purified through reverse-phase chromatography (H₂O, MeCN 10-100%) to yield the desired product.

### General Procedure 3: General Procedure for Amino-Oxadiazole (Scheme GP 3)

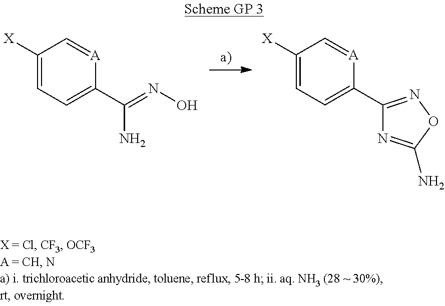

Trichloroacetic anhydride (1.1 equiv.) was added dropwise to a suspension of amidoxime (1.0 equiv.) in toluene at rt and the mixture was refluxed for 5 to 8 h. The volatiles were removed in vacuo thus giving the trichloromethyloxadiazole intermediate (used without further purification), which was then added dropwise to an aqueous solution of NH₃ (28-30%). The mixture was stirred at rt overnight. The solid was filtered and washed with water (3×20 mL) then PE/DCM (1:1, 3×20 mL) thus giving the titled compound.

### General Procedure 4: General Procedure for Buchwald-Hartwig Coupling (Scheme GP 4)

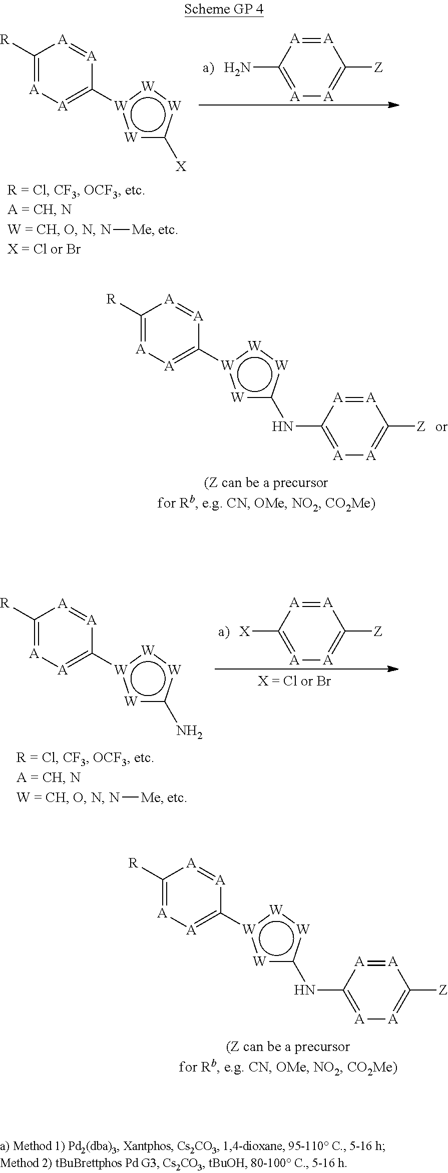

Method 1: A re-sealable Schlenk tube was charged with amine (1.0 eq.), Pd₂(dba)₃ (0.02-0.20 eq., typically 5 mol %), Xantphos (0.04-0.40 eq., typically 10 mol %), (hetero)arylhalide (typically 0.5-5.0 eq., typically 1.0-1.5 eq.), Cs₂CO₃ (1.5-3.0 eq.), and 1,4-dioxane. After the mixture was degassed and carefully subjected to three cycles of evacuation and backfilling with N₂, the reaction was stirred at 95-110° C. for 5-16 h. The volatiles were evaporated. The mixture was then suspended in H₂O, filtered and washed with H₂O, aq. potassium ethyl xanthate solution (10%) and DCM to give the titled compound. If required, the compound was purified via column chromatography with mixtures of petroleum spirit, DCM, EtOAc and/or MeOH as eluents, or via reverse-phase chromatography.

Method 2: The nitrogen nucleophile, (hetero)arylhalide, Cs₂CO₃ (1.5-3 eq.) and tBuBrettphos Pd G3 (0.02-0.20 eq., typically 5 mol %) precatalyst were placed in a sealable vessel under a N₂ atmosphere, N₂ sparged tBuOH was added and the vessel was sealed and heated to the indicated temperature for the indicated time. Unless otherwise indicated, the reaction was then concentrated, diluted with water and EtOAc, brine was added as required to aid phase separation, the phases were separated, the organic layer was washed with brine, dried with MgSO₄, filtered and concentrated onto silica before purification with silica gel flash chromatography using the indicated solvents, typically mixtures of petroleum spirit, DCM, EtOAc and/or MeOH.

General Procedure 5: Oxazole Formation from Azido Ketone (Scheme GP 5)

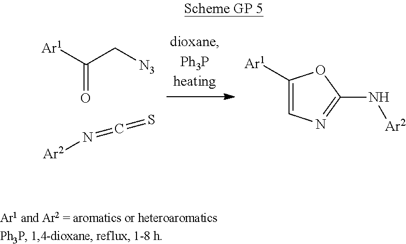

A solution of the appropriate 2-azido-ketone (1 eq.), and isothiocyanate (1 eq.) and PPh₃ (1 eq.) in dry 1,4-dioxane (0.15 M) was heated to 90° C. for the indicated time, typically from 1 to 8 h. On cooling, the mixture was concentrated to a solid and triturated with organic solvents, e.g. DCM, EtOAc or Et₂O. The product was collected by filtering and further washing with the same organic solvent. If required, the product can be purified on silica gel or reversed phase (C18).

### General Procedure 6: Isothiocyanate Formation (Scheme GP 6)

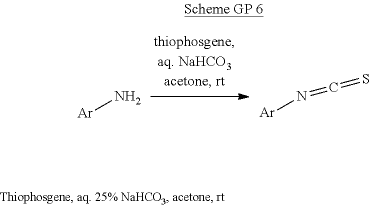

To a suspension of the appropriate aromatic amine (1 eq.) in a 3:2 mixture of acetone:25% NaHCO₃ (aq.) (0.25 M in substrate) was added a solution of thiophosgene (1.2 eq.) in acetone (1.5 M) at 0° C. and the mixture was stirred at rt for the indicated time, typically from 1 to 24 h. Reaction progress was monitored by ¹H NMR, LCMS, or TLC. Once the starting amine has been completely consumed, the mixture was diluted with EtOAc and washed with H₂O. The organic layer was dried over MgSO₄ and concentrated to give the crude product, which in most instances can be used without purification. If required, the crude isothiocyanate can be further purified on silica gel using a mixture of petroleum spirits and EtOAc or DCM and EtOAc to furnish the pure product.

### General Procedure 7: Oxadiazole Formation (Scheme GP 7)

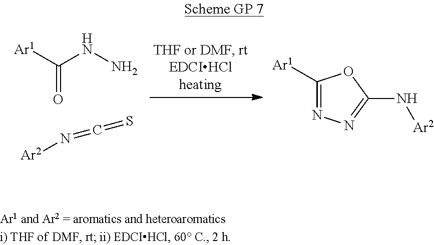

A DMF or THF solution of the appropriate aromatic isothiocyanate (1 eq., 0.2 to 0.5 M concentration) and hydrazide (1 eq.) were stirred at rt for the indicated time, typically from 2 to 18 h. EDCI·HCl (1.2 equiv.) was then added and the mixture heated at 60° C. for 2 h. On cooling, water (1-2 folds volume of organic solvents) was added and the mixture stirred at rt for 0.5 h. The resulting precipitate was filtered and washed with water and occasionally with organic solvents, e.g. DCM or Et₂O, solubility permitting, to provide the desired oxadiazole.

General Procedure 8: Alkyl Deprotection with BBr₃ (Scheme GP 8)

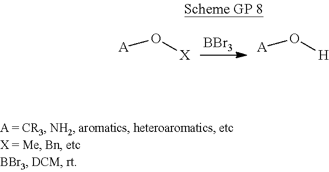

BBr₃ (3-20 equivalents as a commercially available solution in organic solvent or neat) was added dropwise to a solution of the protected substrate in dry DCM (0.2 to 0.5 M concentrations) under N₂ at 0° C. The resultant reaction was stirred at rt for the indicated time, typically from 2 to 18 h, sat. aq. NaHCO₃ solution was then poured into the flask to quench BBr₃ and the mixture was stirred for 2 h. Occasionally most of the organic solvents were removed in vacuo prior to quenching with sat. NaHCO₃ solution. After removing the volatile solvents, products were isolated with filtering and washing with water. In some instances, the product was purified using preparative HPLC.

### General Procedure 9: Isoxazole Formation (Scheme GP 9)

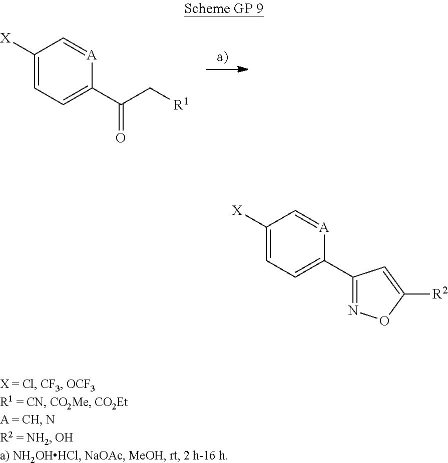

NH₂OH·HCl (3.0 equiv.) and NaOAc (3.0 equiv.) were stirred in CH₃OH at room temperature for 1 h, and then β-ketonitrile/-ester (1.0 equiv.) was added to the mixture. The reaction mixture was left to react until the starting material was consumed completely as judged by TLC detection. Water was added to the reaction which was extracted with ethyl acetate, and the organic layer was washed with brine and dried with anhydrous Na₂SO₄. The crude reaction mixture was purified by column chromatography on silica gel with mixtures of petroleum spirit, DCM, EtOAc and/or MeOH.

### General Procedure 10: Chloro-Isoxazole Formation (Scheme GP 10)

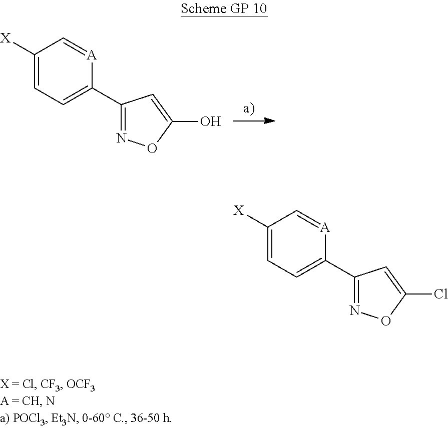

Triethylamine (0.6 equiv.) was added dropwise at 0° C. to a stirring suspension of the isoxazol-5(4H)-one (1.0 equiv.) in POCl₃ (10 equiv.). The mixture was stirred at 60° C. for the indicated time, typically from 36 to 50 h, poured into ice, carefully basified to a pH of 6-7 by addition of 10% aq. KOH, and extracted with DCM. The organic layer was dried over Na₂SO₄ and filtered. The solvent was evaporated, and the product was purified by column chromatography (petroleum spirits:EtOAc, 10:1) to give the desired chloroisoxazole.

General Procedure 11: PMB Deprotection with TFA (Scheme GP 11)

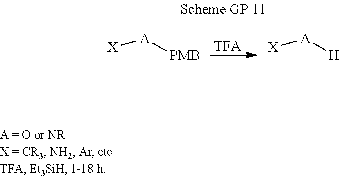

The protected substrate was stirred in a mixture of TFA (0.2-0.5 M concentration) and Et₃SiH (5% volume) at the indicated temperature, typically at rt, but occasionally at reflux. In some instances, anisole was used instead of Et₃SiH. The reaction progress was monitored by LCMS. Upon complete consumption of the starting material, typically after 1 to 18 h, the volatile solvents were evaporated in vacuo to dryness the residue was purified on preparative HPLC to provide the desired deprotected product. Alternatively, when the reaction results in a suspension, the product may be isolated filtering, then washing with water, and organic solvents, solubility permitting.

### General Procedure 12: Suzuki Coupling (Scheme GP 12)

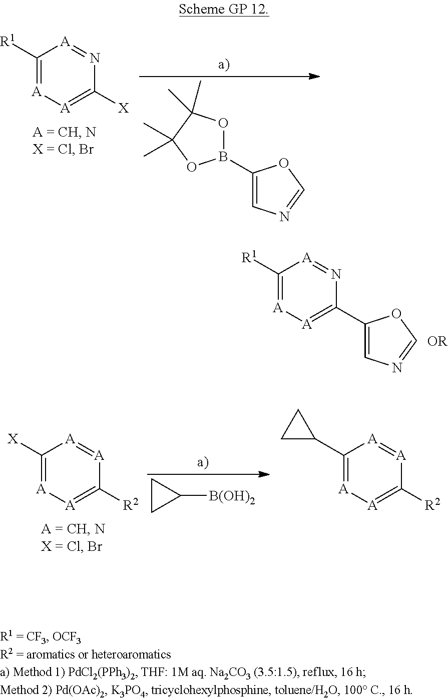

Method 1: To a degassed biphasic solution of THF (3.5 mL) and 1 M aq. Na₂CO₃ (1.5 mL), the (hetero)arylhalide (1.0 eq.), 5-(4,4,5,5-tetramethyl-1,3,2-dioxaborolan-2-yl)oxazole (1.0 M in THF, 1.1 eq.) and PdCl₂(PPh₃)₂ (0.1 eq.) and the mixture heated to 100° C. and stirred for 16 h. Upon completion, the reaction mixture was diluted with EtOAc and the organic layer filtered through cotton wool, and washed with sat. NaHCO₃. The organic layer was dried over anhydrous MgSO₄ and concentrated in vacuo to yield the crude product. The crude product subjected to purification via flash column chromatography (PhMe, 0-20% EtOAc) to give the desired product.

Method 2: To a solution of (hetero)arylhalide (1.0 equiv.), boronic acid (1.3 equiv.), potassium phosphate (3.0 equiv.) and tricyclohexylphosphine (0.01 equiv.) in toluene/water (10:1, 0.1-0.2 M) under a nitrogen atmosphere was added Pd(OAc)₂ (0.005 equiv.). The mixture was heated to 100° C. overnight and then cooled to room temperature. Water was added and the mixture extracted with EtOAc, the combined organics were washed with brine, dried over MgSO₄ and concentrated in vacuo. Purification by column chromatography afforded the desired compound.

### General Procedure 13: Synthesis of Aldehyde (Scheme GP 13)

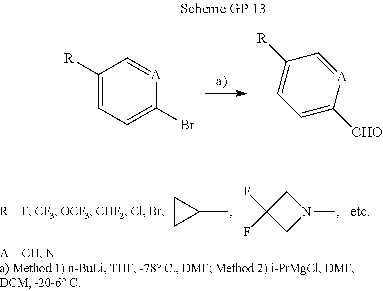

Method 1: n-BuLi (1.6 M in hexane, 1.1 equiv.) was added to a solution of bromide (1.0 equiv.) in dry THF (0.5 M) over 10 min at −78° C. under nitrogen atmosphere. After a further 0.5 h, DMF (3.0-6.0 equiv.) was added and the mixture was stirred for 1 h at −78° C. then brought to 0° C. over 2 h. Saturated aq. NH₄Cl solution was added and the aqueous phase was extracted with EtOAc. The combined organic phases was dried over MgSO₄, filtered, concentrated and subjected to flash chromatography on silica gel (2%-10%, EtOAc/petroleum spirits).

Method 2: i-PrMgCl (2 M in Et₂O, 1.15 equiv.) was added to the bromide (1.0 equiv.) in DCM (0.3-0.5 M) at −2° C. over 3 min. After stirring at 0-6° C. for 40 min, the mixture was cooled to −20° C. and DMF (2.0 equiv.) was added in one portion. The mixture was warmed to 0° C. over 20 minutes then quenched by addition of saturated aq. NaHCO₃ in one portion then filtered through a celite pad, extracted with EtOAc, dried over Na₂SO₄, concentrated under reduced pressure to give the desired product. If required, the compound was purified via column chromatography with mixtures of petroleum spirit, DCM, EtOAc and/or MeOH.

### General Procedure 14: Synthesis of Oxazole (Scheme GP 14)

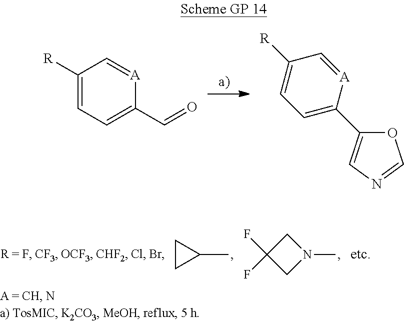

A suspension of aryl aldehyde (1.0 equiv.), K₂CO₃ (1.2 equiv.), TosMIC (1.1 equiv.) in MeOH was heated to reflux for the indicated time. Volatiles were removed in vacuo, then H₂O was added, followed by an extraction with Et₂O. The combined organic layer was dried over MgSO₄ and concentrated under reduced pressure. The resultant crude residue was subjected to flash chromatography on silica gel (3%-20%, EtOAc/petroleum spirits) to give the indicated oxazole.

General procedure 15: Halogenation of oxazole (Scheme GP 15)

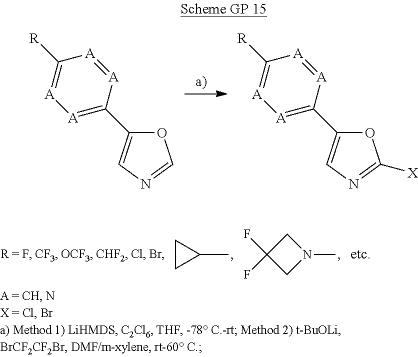

Method 1: LiHMDS (1.0 M in THF, 1.05-1.2 equiv.) was added to a solution of oxazole (1.0 equiv.) in dry THF (0.1-0.2 M) at −78° C. under N₂ atmosphere. After a further 0.5 h, a solution of C₂Cl₆ (1.5 equiv.) in THF (2 M) was added. The resulting reaction mixture was stirred at −78° C. for another 2 h and allowed to warm to rt over 14 h. The reaction was quenched with saturated aq. NaHCO₃ solution. The aqueous phase was extracted with EtOAc. The combined organic phases were dried over Na₂SO₄, filtered, concentrated and subjected to flash chromatography on silica gel (2%-10%, EtOAc/petroleum spirits).

Method 2: BrCF₂CF₂Br (2.0 equiv.) and t-BuOLi (2.0 equiv.) were added to a solution of oxazole (1.0 equiv.) in DMF/m-xylene (1:1 ratio, 0.3-0.5 M). The resulting mixture was stirred at 60° C. for 3 h and quenched with saturated aq. NaHCO₃ solution. The aqueous phase was extracted with EtOAc. The combined organic phases were dried over Na₂SO₄, filtered, concentrated and subjected to flash chromatography on silica gel (3%-10%, EtOAc/petroleum spirits).

¹H NMR spectra were recorded at 400 MHz. ¹³C NMR spectra were recorded at 101 MHz. All chemical shifts were calibrated using residual non-deuterated solvent (e.g. chloroform) as an internal reference and are reported in parts per million (6) relative to trimethylsilane (6=0). Thin layer chromatography (TLC) was performed using 0.25 mm thick plates pre-coated with Merck Kieselgel 60 F254 silica gel, and visualised using UV light (254 nm and 365 nm). Liquid chromatography mass spectrometry (LCMS) was performed using either APCI or ESI LCMS. Each method used 254 nm and 214 nm detectors and a reverse phase C₈(2) 5 50×4.6 mm 100 A column. The column temperature was 30° C. The eluent system used was solvent A (H₂O with 0.1% formic acid) and solvent B (MeCN with 0.1% formic acid). LCMS (ESI) method: the gradient starts from [95% solvent A/5% solvent B] for 1 minute, reaches [100% solvent B] over 1.5 min, maintained for 1.3 min, and then changed to [95% solvent A/5% solvent B] over 1.2 min. High resolution mass spectra (HRMS) were recorded on both a time-of-flight mass spectrometer fitted with either an electrospray (ESI) ion or atmospheric pressure chemical ionisation (APCI) source, the capillary voltage was 4000 V or on an exactive mass spectrometer fitted with an ASAP ion source.

Without limiting the disclosure by theory, in some embodiments the compounds described herein may interact with Des1 and be useful in treating a disease or condition mediated by Des1 activity. As used herein, the term “interact” when used at least in the context of the compounds of the disclosure, includes an association of the compound with the enzyme so as to partially or fully inhibit, retard or prevent biochemical activity of the enzyme, (e.g. introduction of the Δ4 double bond into dihydroceramide to generate ceramide). This may occur through any means such as chemically or associatively binding at one or more sites of the enzyme, promoting reaction with other endogenous molecules or associating in such a manner so as to cause degradation or a conformational change in the enzyme. Determination of the interaction of the compounds with one or more enzymes may be determined in accordance with any suitable methods of the art, including methods which measure enzyme activity inhibition, such as the procedures described in the Examples. In some embodiments, a compound may be considered to interact with an enzyme if, in accordance with the procedure used, it demonstrates at least a measurable or otherwise determinable level of enzyme activity inhibition. Selective interaction, e.g. selective inhibition, refers to the interaction of a compound with an enzyme and/or binding site thereof in complete or partial preference over another enzyme and/or binding site.

In some embodiments, the compounds may selectively inhibit one Des isoform over the other. For example, one or more compounds may be selective Des1 inhibitors. In other embodiments, one or more compounds may be selective Des2 inhibitors.

In further embodiments, one or more compounds may exhibit an IC₅₀ with respect to Des (e.g. Des1 and/or Des2) activity of less than about 100 μM. In further embodiments, one or more compounds may exhibit an IC₅₀ with respect to Des (e.g. Des1 and/or Des2) activity of less than about 50 μM. In further embodiments, one or more compounds may exhibit an IC₅₀ with respect to Des (e.g. Des1 and/or Des2) activity in the range of less than about 10-5 μM. In further embodiments, one or more compounds may exhibit an IC₅₀ with respect to Des (e.g. Des1 and/or Des2) activity of less than about 1 μM. In further embodiments, one or more compounds may exhibit an IC₅₀ with respect to Des (e.g. Des1 and/or Des2) activity of less than about 1 μM. In still further embodiments, one or more compounds may exhibit an IC₅₀ with respect to Des (e.g. Des1 and/or Des2) activity of less than about 0.1 μM. In still further embodiments, one or more compounds may exhibit an IC₅₀ with respect to Des (e.g. Des1 and/or Des2) activity of less than about 0.01 μM. In still further embodiments, one or more compounds may exhibit an IC₅₀ with respect to Des (e.g. Des1 and/or Des2) activity of less than about 0.001 μM. In still further embodiments, one or more compounds may exhibit an IC₅₀ with respect to Des (e.g. Des1 and/or Des2) activity of less than about 0.0001 μM. In still further embodiments, one or more compounds may exhibit an IC₅₀ with respect to Des (e.g. Des1 and/or Des2) activity in the range of about 1.0-10 μM, or 0.1-1.0 μM, or 0.01-0.1 μM, or 0.001-0.01 μM, or 0.0001-0.001 μM.

In some embodiments, one or more compounds of Formula (I′) and/or (II″) demonstrate inhibitory activity of Des (e.g. Des1 and/or Des2) that makes them useful in the treatment and/or prevention of diseases mediated by undesirable or excessive Des activity and/or where the inhibition of Des enzyme activity is therapeutically beneficial. In some embodiments, compounds of Formula (I′) and/or (II″) demonstrate inhibitory activity of Des1. In some embodiments Des activity, such as Des1 activity, may be linked to fibrosis or proliferative diseases. Thus, without limiting the present disclosure by theory, in some embodiments Des inhibition, such as Des1 inhibition, may be useful in treating fibrosis or a proliferative disease. In some embodiments, compounds of the disclosure demonstrate antiproliferative activity and/or demonstrate anti-fibrotic activity.

Some compounds with Des1 inhibitory have also been shown to have anti-proliferative activity (see Aurelio, L. et al, supra). In some embodiments, one or more compounds of Formulae (I′) and/or (II″) demonstrate antiproliferative activity. In still further example, the antiproliferative activity may be observed against a single cell line or type, or may be observed in two or more different cell lines or cancer types. Thus, one or more compounds of the disclosure may be useful in therapy against a single cancer type or two or more cancer types.

In some embodiments, the compounds of the disclosure, may be useful in treating a disease or condition mediated by Des1, for example in which excessive or undesirable Des1 activity is implicated, such as where undesirable cell proliferation is involved, including the treatment or inhibition of cancer and/or metastases, or the treatment of fibrotic diseases, and may be administered to a subject in a treatment or inhibiting effective amount. A treatment or inhibiting effective amount is intended to include an amount which, when administered according to the desired dosing regimen, at least partially attains the desired therapeutic treatment or inhibiting effect, and may include one or more of: alleviating, eliminating or reducing the frequency of occurrence of one or more symptoms of, preventing or delaying the onset of, inhibiting the progression of, or halting or reversing (partially or altogether) the onset or progression of the particular disorder or condition, or pathology thereof, being treated. As used herein, “inhibiting undesirable cell proliferation”, includes preventing, arresting, retarding the rate or extent of, or otherwise delaying or reversing excessive, uncontrolled, detrimental or otherwise undesirable cell proliferation, such as may occur in cancer growth or metastasis.

In some embodiments, compounds of the disclosure and/or their salts or solvates may therefore be useful as anti-proliferative agents e.g. in treating undesirable cell proliferation as, such as found in cancerous conditions, including hormone-related cancers, such as breast cancer and prostate cancer, and their metastasis. Other cancerous conditions which may be amenable to treatment by the compounds described herein include lung, colon, pancreatic and brain cancer as well as lymphoma. The compounds described herein may have utility in treating primary cancers and/or treating or inhibiting metastases (i.e. secondary cancers).

In some embodiments, compounds of the disclosure, including their pharmaceutically acceptable salts and solvates, may be useful in the treatment of fibrosis and/or fibrotic diseases. As used herein, fibrosis and fibrotic disease includes the formation of excess fibrous connective tissue in an organ or tissue, such as a reactive or reparative response to an injury, such as organ transplant, or pathological state, such as inflammation, which can interfere with normal organ or tissue function. Fibrosis may be found in vascular, heart, lung, liver, skin or kidney tissue, and may include pulmonary fibrosis, liver cirrhosis, systemic sclerosis, progressive kidney disease and cardiac fibrosis associated with various cardiovascular diseases.

Some examples of fibrotic diseases or conditions that may be treated by one or more embodiments of the disclosure include: pulmonary (lung) fibrosis, including idiopathic pulmonary fibrosis and cystic fibrosis; liver fibrosis, (cirrhosis), for example, resulting from chronic liver disease, or hepatitis B, C or D virus infection; cardiac (heart) fibrosis, including endomyocardial fibrosis, atrial fibrosis, and fibrosis resulting from myocardial infarction (heart attack); kidney fibrosis, such as resulting from diabetic nephropathy; primary biliary cirrhosis, gall bladder fibrosis, skin or dermal fibrosis, such as scleroderma, hypertrophic scarring and keloids; bone marrow fibrosis, and intestinal fibrosis, such as Crohn's disease.

In some embodiments, one or more compounds of the disclosure, for example, which inhibit or otherwise interact with Des 1, including their pharmaceutically acceptable salts and solvates, may be useful in the treatment of metabolic disease, in particular non-alcoholic fatty liver disease and non-alcoholic steatohepatitis (NASH) (NAFLD/NASH) (see for example B. Chaurasia et al., Science, 2019, 365(6451), 386-392). Further examples of diseases which may be treated by one or more compounds of the disclosure may include cardiovascular disease, hypertension, type-2 diabetes, cystic fibrosis, chromic kidney disease, diabetic nephropathy, scleroderma, cancer (including, but not limited, prostate cancer, breast cancer, brain cancer, hepatocellular carcinoma, multiple myeloma, acute lymphoid myeloma and colon cancer), neurodegenerative disease, iodiopathic pulmonary fibrosis, chronic obstructive pulmonary disease, and viral disease (Magaye, R., R. et al. Cell. Mol. Life Sci. 2019, 76, 1107-1134; Vieira, C. R. et al., Chem. Biol. 2010, 17, 766-775).

Subjects to be treated include mammalian subjects: humans, primates, livestock animals (including cows, horses, sheep, pigs and goats), companion animals (including dogs, cats, rabbits, guinea pigs), and captive wild animals. Laboratory animals such as rabbits, mice, rats, guinea pigs and hamsters are also contemplated as they may provide a convenient test system. Non-mammalian species such as birds, amphibians and fish may also be contemplated in certain embodiments of the invention.

Suitable dosage amounts and dosing regimens can be determined by the attending physician and may depend on the particular condition being treated, the severity of the condition as well as the general age, health and weight of the subject. Suitable dosage amounts may lie in the range of from 1 μg to 1 g of compound, salt or solvate, for example, 1 μg-1 mg (such as 100 μg, 250 μg, 500 μg, 750 μg), 1 mg-10 mg (such as 2, 5 or 7 mg), 10 mg-50 mg (such as 15, 20, 25, 30, or 40 mg), 50 mg-100 mg (such as 60, 70, 80, 90 mg) or 100 mg-500 mg (such as 200, 250, 300, 400 mg). Dosages may be administered once, or multiple times daily (e.g. 2, 3, or 4 times), or one or more times weekly, fortnightly or monthly. Administration may be over a limited period of time to treat an acute disorder or condition, for example 1, 2, 3, or 4 weeks, or 2 or 3 months, or may occur over extended periods to treat a chronic disorder or condition, for example greater than 3 months, e.g. 6 or 12 months, 1-2 years or longer.

The active ingredient may be administered in a single dose or a series of doses. While it is possible for the active ingredient to be administered alone, it is preferable to present it as a composition, preferably as a pharmaceutical composition, with one or more pharmaceutically acceptable additives. The compounds may also be packaged or presented as a combination with one or more other therapeutic agents and/or anti-proliferative or anti-cancer agents. The components of the combinations may be administered in conjunction with each other, either contemporaneously or at separate times, as a single composition or separate compositions, as appropriate. Thus, the compositions contemplated herein may contain the compounds of the disclosure, or a pharmaceutically acceptable salt or solvate thereof, as the only therapeutic agent or anti-proliferative/anti-cancer or anti-fibrotic agent, or may further contain one or more additional therapeutic or anti-proliferative/anti-cancer or anti-fibrotic agents. Thus, the present disclosure also relates to the use of a compound of Formula (I′) or (I″) or a pharmaceutically acceptable salt or solvate thereof in the manufacture of a medicament for treating a disease or condition in which excessive or undesirable sphingosine kinase activity is implicated, or inhibiting undesirable cell proliferation, e.g. in treating cancer or inhibiting or preventing metastasis, or treating fibrotic diseases.

The formulation of such compositions is well known to those skilled in the art, see for example, Remington's Pharmaceutical Sciences, 21^{st} Edition, Mack Publishing, 2005. The composition may contain any suitable additives, carriers, diluents or excipients. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

The additive must be pharmaceutically acceptable in the sense of being compatible with the other ingredients of the composition and not injurious to the subject. Compositions include those suitable for oral, rectal, nasal, topical (including dermal, buccal and sublingual), vaginal or parental (including subcutaneous, intramuscular, intravenous and intradermal) administration. The compositions may conveniently be presented in unit dosage form and may be prepared by any methods well known in the art of pharmacy. Such methods include the step of bringing into association the active ingredient with the additive which constitutes one or more accessory ingredients. In general, the compositions are prepared by uniformly and intimately bringing into association the active ingredient with liquid additive or finely divided solid additive or both, and then if necessary shaping the product.

Compositions comprising a compound of the disclosure may be administered to a subject via any suitable method, including, orally, parenterally, topically, rectally, vaginally, or by inhalation.

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. inert diluent), preservative disintegrant (e.g. sodium starch glycolate, cross-linked polyvinyl pyrrolidone, cross-linked sodium carboxymethyl cellulose) surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured base, usually sucrose and acacia or tragacanth gum; pastilles comprising the active ingredient in an inert basis such as gelatin and glycerin, or sucrose and acacia gum; and mouthwashes comprising the active ingredient in a suitable liquid carrier.

Compositions suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Devices for transdermal delivery, such as patches, may also be used to administer the compounds of the invention.

Compositions for rectal administration may be presented as a suppository with a suitable base comprising, for example, cocoa butter, glycerin, gelatin or polyethylene glycol.

Compositions suitable for vaginal administration may be presented as pessaries, tampons, creams, gels, pastes, foams or spray formulations containing in addition to the active ingredient such carriers as are known in the art to be appropriate.

Compositions suitable for parenteral administration include aqueous and non-aqueous isotonic sterile injection solutions which may contain anti-oxidants, buffers, bactericides and solutes which render the composition isotonic with the blood of the intended recipient; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage compositions are those containing a daily dose or unit, daily sub-dose, as herein above described, or an appropriate fraction thereof, of the active ingredient.

It should be understood that in addition to the active ingredients particularly mentioned above, the compositions of this disclosure may include other additives or agents conventional in the art having regard to the type of composition in question, for example, those suitable for oral administration may include such further agents as binders, sweeteners, thickeners, flavouring agents disintegrating agents, coating agents, preservatives, lubricants and/or time delay agents. Suitable sweeteners include sucrose, lactose, glucose, aspartame or saccharine. Suitable disintegrating agents include corn starch, methylcellulose, polyvinylpyrrolidone, xanthan gum, bentonite, alginic acid or agar. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

The present disclosure also relates to prodrugs of Formula (I′) and (I″). Any compound that is a prodrug of a compound of Formula (I′) or (I″) is within the scope and spirit of the invention. The term “prodrug” is used in its broadest sense and encompasses those derivatives that are converted in vivo, either enzymatically or hydrolytically, to the compounds of the invention. Such derivatives would readily occur to those skilled in the art, and include, for example, compounds where a free thiol or hydroxy group is converted into an ester, such as phosphonate, sulphonate and carboxy esters, such as an acetate, or thioester or where a free amino group is converted into an amide such as a carboxy, phosphonate or sulphonate amide. Procedures for acylating the compounds of the invention, for example to prepare ester and amide prodrugs, are well known in the art and may include treatment of the compound with an appropriate carboxylic acid, anhydride or chloride in the presence of a suitable catalyst or base. Esters of carboxylic acid (carboxy) groups are also contemplated. Suitable esters C₁₋₆ alkyl esters; C₁₋₆ alkoxymethyl esters, for example methoxymethyl or ethoxymethyl; C₁₋₆alkanoyloxymethyl esters, for example, pivaloyloxymethyl; phthalidyl esters; C₃₋₈ cycloalkoxycarbonylC₁₋₆ alkyl esters, for example, 1-cyclohexylcarbonyloxyethyl; 1,3-dioxolen-2-onylmethyl esters, for example, 5-methyl-1,3-dioxolen-2-onylmethyl; and C₁₋₆ alkoxycarbonyloxyethyl esters, for example, 1-methoxycarbonyloxyethyl. Prodrugs of amino functional groups include amides (see, for example, Adv. BioSci., 1979, 20, 369, Kyncl, J. et al), enamines (see, for example, J. Pharm. Sci., 1971, 60, 1810, Caldwell, H. et al), Schiff bases (see, for example, U.S. Pat. No. 2,923,661 and Antimicrob. Agents Chemother., 1981, 19, 1004, Smyth, R. et al), oxazolidines (see, for example, J. Pharm. Sci, 1983, 72, 1294, Johansen, M. et al), Mannich bases (see, for example, J. Pharm. Sci. 1980, 69, 44, Bundgaard, H. et al and J. Am. Chem. Soc., 1959, 81, 1198, Gottstein, W. et al), hydroxymethyl derivatives (see, for example, J. Pharm. Sci, 1981, 70, 855, Bansal, P. et al) and N-(acyloxy)alkyl derivatives and carbamates (see, for example, J. Med. Chem., 1980, 23, 469, Bodor, N. et al, J. Med. Chem., 1984, 27, 1037, Firestone, R. et al, J. Med. Chem., 1967, 10, 960, Kreiger, M. et al, U.S. Pat. No. 5,684,018 and J. Med. Chem., 1988, 31, 318-322, Alexander, J. et al). Other conventional procedures for the selection and preparation of suitable prodrugs are known in the art and are described, for example, in WO 00/23419; Design of Prodrugs, H. Bundgaard, Ed., Elsevier Science Publishers, 1985; Methods in Enzymology, 42: 309-396, K. Widder, Ed, Academic Press, 1985; A Textbook of Drug Design and Development, Krogsgaard-Larsen and H. Bundgaard, Eds, Chapter 5, p 113-191 (1991); Advanced Drug Delivery Reviews, 8; 1-38 (1992); Journal of Pharmaceutical Sciences, 77; 285 (1988), H. Bundgaard, et al; Chem Pharm Bull, 32692 (1984), N. Kakeya et al and The Organic Chemistry of Drug Design and Drug Action, Chapter 8, pp 352-401, Academic press, Inc., 1992.

Suitable pharmaceutically acceptable salts of compounds of the disclosure may include, but are not limited to salts of pharmaceutically acceptable inorganic acids such as hydrochloric, sulphuric, phosphoric nitric, carbonic, boric, sulfamic, and hydrobromic acids, or salts of pharmaceutically acceptable organic acids such as acetic, propionic, butyric, tartaric, maleic, hydroxymaleic, fumaric, maleic, citric, lactic, mucic, gluconic, benzoic, succinic, oxalic, phenylacetic, methanesulphonic, toluenesulphonic, benzenesulphonic, salicyclic sulphamic, aspartic, glutamic, edetic, stearic, palmitic, oleic, lauric, pantothenic, tannic, ascorbic, fendizoic, 4-4′-methylenebis-3-hydroxy-2-naphthoic acid, o-(p-hydroxybenzoyl)benzoic, 4′-4″-dihydroxytriphenylmethane-2-carboxylic acid and valeric acids. Base salts include, but are not limited to, those formed with pharmaceutically acceptable cations, such as sodium, potassium, lithium, calcium, magnesium, ammonium and alkylammonium. Basic nitrogen-containing groups may be quaternised with such agents as lower alkyl halide, such as methyl, ethyl, propyl, and butyl chlorides, bromides and iodides or dialkyl sulfates such as dimethyl and diethyl sulfate.

An example of a pharmaceutically acceptable salt of any of the compounds described herein in any of the aspects, embodiments or examples is the hydrochloride salt.

The compounds of the disclosure may be in crystalline form either as the free compounds or salts, or as solvates and it is intended that both forms are within the scope of the present disclosure. The term “solvate” refers to a complex or aggregate formed by one or more molecules of a solute, i.e. compounds of the disclosure, and one or more molecules of a solvent. Suitable solvents are well understood in the art and include for example, of water, i.e. to form hydrates, and common organic solvents such as alcohols (MeOH, ethanol, isopropanol) and acetic acid. Methods of solvation are generally known within the art, for example, recrystallization from an appropriate solvent.

It will also be recognised that certain compounds of formula (I′) may possess asymmetric centres and are therefore capable of existing in more than one stereoisomeric form, such as enantiomers and diastereomers. The invention thus also relates to optically active compounds and compounds in substantially pure isomeric form at one or more asymmetric centres, e.g., enantiomers having greater than about 90% ee, such as about 95% or 97% ee or greater than 99% ee, as well as mixtures of isomers, including racemic mixtures, thereof. Such isomers may be prepared by asymmetric synthesis, for example using chiral intermediates, enzymes, or mixtures may be resolved by conventional methods, e.g., chromatography, recrystallization, or use of a resolving agent.

The compounds of the disclosure may also be presented for use in veterinary compositions. These may be prepared by any suitable means known in the art. Examples of such compositions include those adapted for:
oral administration, external application (e.g. drenches including aqueous and non-aqueous solutions or suspensions), tablets, boluses, powders, granules, pellets for admixture with feedstuffs, pastes for application to the tongue;
parenteral administration, e.g. subcutaneous, intramuscular or intravenous injection as a sterile solution or suspension;
topical application e.g. creams, ointments, gels, lotions etc.

The following examples are provided for the purpose of illustrating some embodiments of the disclosure but are not to be construed as limiting the generality hereinbefore described.

### EXAMPLES

### Example 1

### 1. 5-((5-(4-Chlorophenyl)oxazol-2-yl)amino)-N-hydroxypicolinamide (Scheme 1)

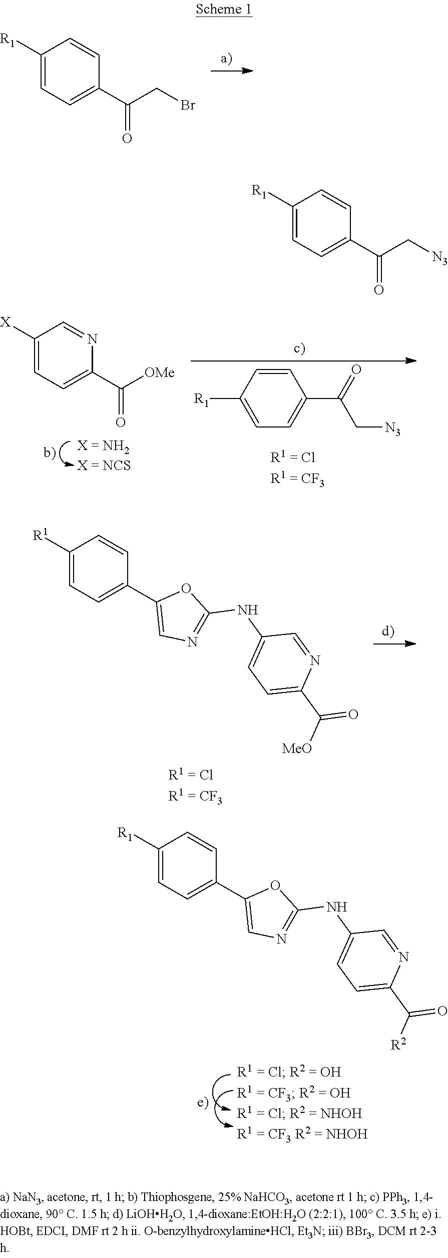

To a suspension of methyl 5-aminopicolinate (0.380 g, 2.50 mmol) in acetone (6 mL) and 25% NaHCO₃ (aq.) (4 mL) was added a solution of thiophosgene (0.230 mL, 3.00 mmol) in acetone (2 mL) at 0° C. and the mixture stirred at rt for 1 h. The mixture was diluted with EtOAc (30 mL) and washed with H₂O (2×15 mL). The organic layer was dried over MgSO₄ and concentrated to give methyl 5-isothiocyanatopicolinate as a cream semi-solid (0.387 g, 80% yield). ¹H NMR (401 MHz, CDCl₃) δ 8.66-8.55 (m, 1H), 8.15 (dd, J=8.4, 0.6 Hz, 1H), 7.64 (dd, J=8.4, 2.4 Hz, 1H), 4.02 (s, 3H). ¹³C NMR (101 MHz, CDCl₃) δ 164.7 (C═O), 147.0 (CH), 145.3 (C), 141.5 (C), 133.2 (CH), 132.8 (C), 126.0 (CH), 53.3 (CH₃). LCMS R_{f} (min)=5.52, MS m/z=195.1 [M+H]⁺.

A solution of 2-bromo-4′-chloroacetophenone (0.20 g, 0.75 mmol) and NaN₃ (0.097 g, 1.5 mmol) in acetone (10 mL) was stirred at rt for 16 h. The reaction mixture was then concentrated in vacuo and the residue redissolved in DCM (30 mL) and washed with H₂O (2×15 mL). The organic layer was dried over MgSO₄ and concentrated to an orange semi-solid (0.14 g). 2-Azido-1-(4-chlorophenyl)ethan-1-one (0.094 g, 0.41 mmol) was then dissolved in dry 1,4-dioxane (4 mL) and methyl 5-isothiocyanatopicolinate (0.080 g, 0.41 mmol) and PPh₃ (0.11 g, 0.41 mmol) added and the mixture heated to 90° C. for 1.5 h. The mixture was then concentrated to a solid and triturated with DCM, providing methyl 5-((5-(4-chlorophenyl)oxazol-2-yl)amino)picolinate as a cream solid (0.089 g, 73% yield). ¹H NMR (401 MHz, DMSO-d₆) δ 11.14 (s, 1H, NH), 8.82 (d, J=2.2 Hz, 1H), 8.30 (dd, J=8.7, 2.7 Hz, 1H), 8.11-8.05 (m, 1H), 7.63 (d, J=8.8 Hz, 2H), 7.62 (s, 1H), 7.52 (d, J=8.7 Hz, 2H), 3.85 (s, 3H). ¹³C NMR (101 MHz, DMSO-d₆) δ 164.9 (C═O), 155.5 (C), 143.8 (C), 139.5 (C), 138.9 (C), 138.5 (CH), 131.7 (C), 129.2 (CH), 126.6 (C), 126.0 (CH), 124.5 (CH), 123.4 (CH), 122.5 (CH), 52.0 (CH₃).

LiOH·H₂O (0.031 g, 0.74 mmol)) in H₂O (0.75 mL) was added to a solution of methyl 5-((5-(4-chlorophenyl)oxazol-2-yl)amino)picolinate (0.081 g, 0.25 mmol) in 1,4-dioxane (1.5 mL) and EtOH (1.5 mL) and the mixture heated at reflux for 3.5 h. Volatiles were removed in vacuo and to the suspension was added NaCl (0.5 g), followed by 1.0 M HCl (aq.) dropwise at 0° C. The resulting precipitate was filtered and washed with H₂O (3 mL), providing 5-((5-(4-chlorophenyl)oxazol-2-yl)amino)picolinic acid as a yellow solid that was recrystallised from H₂O (0.67 g, 86% yield). ¹H NMR (401 MHz, DMSO-d₆) δ 10.87 (s, 1H, NH), 8.73 (d, J=2.4 Hz, 1H), 8.15 (dd, J=8.6, 2.5 Hz, 1H), 7.96 (d, J=8.6 Hz, 1H), 7.62 (d, J=8.7 Hz, 2H), 7.59 (s, 1H), 7.51 (d, J=8.7 Hz, 2H). ¹³C NMR (101 MHz, DMSO-d₆) δ 166.7 (C═O), 155.9 (C), 143.5*(C), 137.4 (CH), 131.6 (C), 129.1 (CH), 126.7*(C), 124.8 (CH), 124.4 (CH), 123.4 (CH), 123.0 (CH).
* Overlapping quaternary carbon resonances

A solution of 5-((5-(4-chlorophenyl)oxazol-2-yl)amino)picolinic acid (0.040 g, 0.13 mmol), HOBt·H₂O (0.021 g, 0.15 mmol) and EDCI·HCl (0.032 g, 0.17 mmol) in dry DMF (1.5 mL) was stirred at rt for 2 h. After this time, O-benzylhydroxylamine hydrochloride (0.10 g, 0.64 mmol) and Et₃N (0.090 mL, 0.64 mmol) were added and the mixture stirred at rt for a further 16 h. DMF was removed in vacuo, washing with toluene (2 mL×3) to aid this process. H₂O (2 mL) was then added and the mixture left to stir at rt for 10 min. The resulting precipitate was filtered and washed with H₂O (1 mL), providing a grey solid (0.035 g) that was chromatographed on silica gel eluting with 25% EtOAc in DCM to afford N-(benzyloxy)-5-((5-(4-chlorophenyl)oxazol-2-yl)amino)picolinamide as a white solid (0.017 g, 32% yield). ¹H NMR (401 MHz, DMSO-d₆) δ 11.83 (s, 1H, NH), 11.02 (s, 1H, NH), 8.76 (d, J=2.6 Hz, 1H), 8.28 (dd, J=8.7, 2.6 Hz, 1H), 7.99 (d, J=8.8 Hz, 1H), 7.63 (d, J=8.7 Hz, 2H), 7.61 (s, 1H), 7.52 (d, J=8.7 Hz, 2H), 7.46 (s, 2H), 7.42-7.32 (m, 3H), 4.93 (s, 2H). ¹³C NMR (101 MHz, DMSO-d₆) δ 161.6 (C═O), 155.7 (C), 143.7 (C), 142.1 (C), 138.5 (C), 137.2 (CH), 136.0 (C), 131.8 (C), 129.2 (CH), 128.8 (CH), 128.3 (CH), 128.3 (CH), 126.6 (C), 124.5 (CH), 123.4 (CH), 123.4 (CH), 123.0 (CH), 77.1 (CH₂). LCMS R_{f} (min)=3.58, MS m/z=420.8 [M+H]⁺.

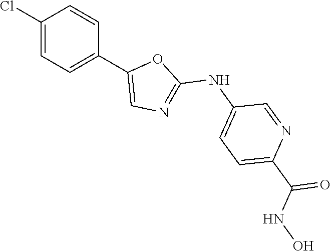 [SMILES: O=C(NO)c1ccc(Nc2ncc(-c3ccc(Cl)cc3)o2)cn1]

BBr₃ 1.0 M in heptane (0.22 mL, 0.22 mmol) was added dropwise to a solution of N-(benzyloxy)-5-((5-(4-chlorophenyl)oxazol-2yl)amino)picolinamide (0.047 g, 0.11 mmol) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The precipitate was filtered and washed with H₂O (10 mL) and Et₂O (2 mL), providing the title compound as a brown solid (0.030 g, 81% yield). Mp 264-269° C. dec. ¹H NMR (401 MHz, DMSO-d₆) δ 11.20 (s, 1H, NH), 10.97 (s, 1H, OH), 8.96 (s, 1H, NH), 8.75 (d, J=2.4 Hz, 1H), 8.26 (dd, J=8.6, 2.6 Hz, 1H), 7.96 (d, J=8.7 Hz, 1H), 7.63 (d, J=8.6 Hz, 2H), 7.61 (s, 1H), 7.52 (d, J=8.7 Hz, 2H). ¹³C NMR (101 MHz, DMSO-d₆) δ 161.4 (C═O), 155.7 (C), 143.6 (C), 142.7 (C), 138.0 (C), 137.1 (CH), 131.6 (C), 129.1 (CH), 126.6 (C), 124.4 (CH), 123.4 (CH), 123.3 (CH), 122.6 (CH). LCMS R_{f} (min)=5.73, MS m/z=329.0 [M−H]⁻. HRMS (ESI) calcd for C₁₅H₁₂ClN₄O₃⁺ [M+H]⁺ 331.0592, found 331.0593.

### 2. N-Hydroxy-5-((5-(4-(trifluoromethyl)phenyl)oxazol-2-yl)amino)picolinamide (Scheme 1) (Intermediate A—2-azido-1-(4-(trifluoromethyl)phenyl)ethan-1-one)

Sodium azide (2 equiv.) was added to a solution of 2-bromo-1-(4-(trifluoromethyl)phenyl)ethan-1-one (0.400 g, 1.498 mmol) in acetone (10 mL) and the mixture stirred at rt for 1 h. On formation of the azide intermediate, the mixture was concentrated to a residue before being partitioned between DCM (30 mL) and H₂O (10 mL). The organic layer was collected and the aq. layer back extracted with DCM (3×20 mL). The combined organic layers were dried over MgSO₄ and concentrated to give 2-azido-1-(4-(trifluoromethyl)phenyl)ethan-1-one as an orange semi-solid (0.327 g, 95.3% yield).

A solution of 2-azido-1-(4-(trifluoromethyl)phenyl)ethan-1-one (0.279 g, 1.22 mmol), methyl 5-isothiocyanatopicolinate (0.250 g, 1.22 mmol) and PPh₃ (0.320 g, 1.22 mmol) in dry 1,4-dioxane (8 mL) was heated to 90° C. for 1.5 h. On cooling, the mixture was concentrated to a solid and triturated with DCM (5 mL), providing methyl 5-((5-(4-(trifluoromethyl)phenyl)oxazol-2-yl)amino)picolinate as an off-white solid (0.256 g, 58% yield). ¹H NMR (401 MHz, DMSO-d₆) δ 11.25 (s, 1H, NH), 8.83 (d, J=2.3 Hz, 1H), 8.31 (dd, J=8.7, 2.7 Hz, 1H), 8.09 (d, J=8.7 Hz, 1H), 7.82 (s, 4H), 7.80 (s, 1H), 3.85 (s, 3H). ¹³C NMR (101 MHz, DMSO-d₆) δ 164.9 (C═O), 156.1 (C), 143.5 (C), 139.7 (C), 138.9 (C), 138.6 (CH), 131.4 (C), 127.1 (C), 126.2 (q, J=3.9 Hz, CF₃), 126.1 (CH), 125.3 (CH), 123.2*(CH), 122.8 (CH), 52.1 (CH₃). LCMS R_{f} (min)=5.98, MS m/z=364.1 [M+H]⁺.
* Overlapping CH resonances (CH from pyridine ring and CH from oxazole)

### Intermediate B—5-((5-(4-(trifluoromethyl)phenyl)oxazol-2-yl)amino)picolinic Acid

LiOH·H₂O (0.052 g, 1.2 mmol)) in H₂O (1 mL) was added to a solution of methyl 5-((5-(4-(trifluoromethyl)phenyl)oxazol-2-yl)amino)picolinate (0.15 g, 0.41 mmol) in 1,4-dioxane (2 mL) and EtOH (2 mL) and the mixture heated at reflux for 3 h. Volatiles were removed in vacuo and to the suspension was added NaCl (0.5 g), followed by 1.0 M HCl (aq.) dropwise at 0° C. The resulting precipitate was filtered and washed with H₂O (3 mL), providing 5-((5-(4-(trifluoromethyl)phenyl)oxazol-2-yl)amino)picolinic acid as a yellow solid that was recrystallised from H₂O (0.12 g, 84% yield). ¹H NMR (401 MHz, DMSO-d₆) δ 11.07 (s, 2H, OH, NH), 8.78 (s, 1H), 8.21 (d, J=8.2 Hz, 1H), 8.00 (d, J=8.1 Hz, 1H), 7.81 (s, 4H), 7.77 (s, 1H). ¹³C NMR (101 MHz, DMSO-d₆) δ 166.3 (C═O), 156.4 (C), 143.6 (C), 138.5 (C), 138.3 (CH), 131.6 (C), 127.6 (C), 127.3 (C), 126.3 (q, J=3.7 Hz, CF₃), 125.8 (CH), 125.4 (CH), 123.4*(CH), 123.2 (CH).
*Overlapping CH resonances (CH from pyridine ring and CH from oxazole)

A solution of 5-((5-(4-(trifluoromethyl)phenyl)oxazol-2-yl)amino)picolinic acid (0.050 g, 0.14 mmol), HOBt·H₂O (0.022 g, 0.17 mmol) and EDCI·HCl (0.034 g, 0.18 mmol) in dry DMF (2 mL) was stirred at rt for 2 h. After this time, O-benzylhydroxylamine hydrochloride (0.11 g, 0.69 mmol) and Et₃N (0.10 mL, 0.69 mmol) were added and the mixture stirred at rt for a further 16 h. The mixture was then diluted with EtOAc (20 mL) and washed with H₂O (3×10 mL). The combined organic layers were dried over MgSO₄ and concentrated to a grey solid (0.092 g) that was triturated with petroleum spirit (4 mL), providing N-(benzyloxy)-5-((5-(4-(trifluoromethyl)phenyl)oxazol-2-yl)amino)picolinamide as a white solid (0.024 g, 39% yield). ¹H NMR (401 MHz, DMSO-d₆) δ 11.84 (s, 1H, NH), 11.12 (s, 1H, NH), 8.77 (s, 1H), 8.34-8.25 (m, 1H), 8.00 (d, J=8.7 Hz, 1H), 7.82 (s, 4H), 7.79 (s, 1H), 7.47 (d, J=6.7 Hz, 2H), 7.43-7.31 (m, 3H), 4.93 (s, 2H). ¹³C NMR (101 MHz, DMSO-d₆) δ 161.5 (C═O), 156.2 (C), 143.3 (C), 142.2 (C), 138.4 (C), 137.2 (CH), 136.0 (C), 131.5 (C), 128.7 (CH), 128.3 (CH), 128.2 (CH), 127.0 (C), 126.1 (q, J=3.8 Hz, CF₃), 125.3 (CH), 123.5 (CH), 123.1*(CH), 123.0 (CH), 77.1 (CH₂). LCMS R_{f} (min)=3.58, MS m/z=454.8 [M+H]⁺.
* Overlapping CH resonances (CH from pyridine ring and CH from oxazole)

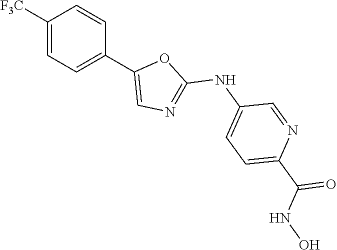 [SMILES: O=C(NO)c1ccc(Nc2ncc(-c3ccc(C(F)(F)F)cc3)o2)cn1]

BBr₃ 1.0 M in heptane (0.33 mL, 0.33 mmol) was added to a solution of N-(benzyloxy)-5-((5-(4-(trifluoromethyl)phenyl)oxazol-2-yl)amino)picolinamide (0.050 g, 0.11 mmol) in dry DCM (1.5 mL) at 0° C. and the mixture stirred at rt for 3 h. Volatiles were removed in vacuo and the residue suspended in sat. NaHCO₃ (aq.) (3 mL) and stirred at rt for 20 min. The precipitate was filtered and washed with H₂O (10 mL) and Et₂O (2 mL), providing the title compound as a yellow solid (0.021 g, 53% yield). Mp 184-188° C. ¹H NMR (401 MHz, DMSO-d₆) δ 11.22 (d, J=1.9 Hz, 1H, NH), 11.07 (s, 1H, OH), 8.97 (d, J=2.0 Hz, 1H, NH), 8.76 (d, J=2.3 Hz, 1H), 8.31-8.24 (m, 1H), 7.97 (d, J=8.7 Hz, 1H), 7.81 (s, 4H), 7.78 (s, 1H). ¹³C NMR (101 MHz, DMSO-d₆) δ 161.4 (C═O), 156.3 (C), 143.2 (C), 142.8 (C), 137.9 (C), 137.2 (CH), 131.5 (C), 127.3 (C), 126.1 (q, J=3.9 Hz, CF₃), 125.3 (CH), 123.5 (CH), 123.1*(CH), 122.6 (CH). LCMS R_{f} (min)=3.66, MS m/z=364.8 [M+H]⁺. HRMS (ESI) calcd for C₁₆H₁₁F₃N₄O₃⁺ [M+H]⁺ 365.0856, found 365.0856.
* Overlapping CH resonances (CH from pyridine ring and CH from oxazole)

### 3. N′-Hydroxy-6-((5-(4-(trifluoromethyl)phenyl)oxazol-2-yl)amino)nicotinimidamide Hydrochloride (Scheme 2)

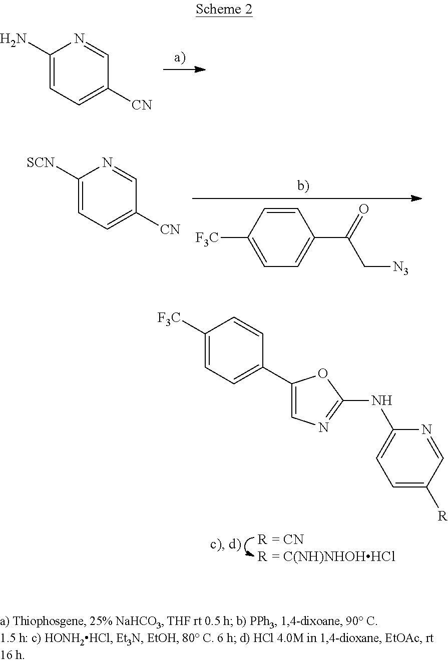

To a suspension of 6-amino-3-pyridinecarbonitrile (0.300 g, 2.52 mmol) in THF (4 mL) and 25% NaHCO₃ (aq.) (5 mL) was added a solution of thiophosgene (0.210 mL, 2.77 mmol) in THF (1 mL) at 0° C. and the mixture stirred at rt for 0.5 h. The mixture was then diluted with DCM (20 mL) and washed with H₂O (2×10 mL). The combined organic layers were dried over MgSO₄ and concentrated to a dark brown oil that was chromatographed on silica gel eluting with 100% DCM, providing 6-isothiocyanatonicotinonitrile as a light brown semi-solid (0.117 g, 29% yield). ¹H NMR (401 MHz, CDCl₃) δ 8.71 (s, 1H), 7.97 (dd, J=8.3, 2.3 Hz, 1H), 7.16 (d, J=8.3 Hz, 1H). ¹³C NMR (101 MHz, CDCl₃) δ 153.3 (CH), 149.9 (C), 145.9 (C), 142.0 (CH), 119.5 (CH), 116.0 (C), 107.9 (C). LCMS R_{f} (min)=3.11, MS m/z=161.9 [M+H]⁺.

A solution of 2-azido-1-(4-(trifluoromethyl)phenyl)ethan-1-one (Intermediate A) (0.074 g, 0.33 mmol), 6-isothiocyanatopicolinonitrile (0.052 g, 0.32 mmol) and PPh₃ (0.085 g, 0.32 mmol) in dry 1,4-dioxane (3 mL) was heated to 90° C. for 1.5 h. On cooling, the mixture was concentrated to a brown solid that was triturated with DCM (4 mL), providing 6-((5-(4-(trifluoromethyl)phenyl)oxazol-2-yl)amino)nicotinonitrile as a white solid (0.11 g, 100% yield). ¹H NMR (401 MHz, DMSO-d₆) δ 11.77 (s, 1H, NH), 8.74 (d, J=1.6 Hz, 1H), 8.24 (dd, J=8.9, 2.2 Hz, 1H), 8.16 (d, J=9.1 Hz, 1H), 7.83 (s, 4H), 7.82 (s, 1H). ¹³C NMR (101 MHz, DMSO-d₆) δ 155.2 (C), 154.3 (C), 152.3 (CH), 143.9 (C), 141.9 (CH), 131.3 (C), 126.1 (q, J=3.9 Hz, CF₃), 125.1 (CH), 123.3*(CH), 122.8 (C), 117.7 (C), 110.3 (CH), 101.2 (C). LCMS R_{f} (min)=3.24, MS m/z=129.0 [M−H]⁻.
* Two overlapping CH resonances (CH from pyridine ring and CH from oxazole ring)

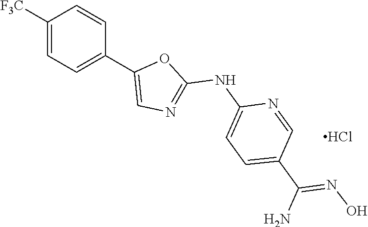 [SMILES: N/C(=N\O)c1ccc(Nc2ncc(-c3ccc(C(F)(F)F)cc3)o2)nc1 ; Cl]

A solution of 6-((5-(4-(trifluoromethyl)phenyl)oxazol-2-yl)amino)nicotinonitrile (0.050 g, 0.15 mmol), HONH₂·HCl (0.042 g, 0.60 mmol) and Et₃N (0.085 mL, 0.60 mmol) in EtOH (3 mL) was heated at reflux for 16 h. On cooling, the mixture was diluted with H₂O (5 mL) and the resulting precipitate filtered and washed with H₂O (3 mL), providing a cream solid (0.045 g). This material was then salted by diluting in EtOAc (1 mL) and adding 4.0 M HCl in 1,4-dioxane (0.030 mL, 0.12 mmol) and stirring at rt for 16 h. The resulting precipitate was filtered and washed with EtOAc (3 mL), affording a yellow solid (0.027 g) that was further purified using RP-HPLC in an AA 30-100B solvent system to give the title compound as a pale yellow solid (0.012 g, 22% yield). Mp 257-261° C. ¹H NMR (401 MHz, DMSO-d₆) δ 11.24 (s, 1H, NH), 9.77 (s, 1H, OH), 8.58 (s, 1H), 8.05 (s, 2H), 7.81 (s, 4H), 7.77 (s, 1H), 6.04 (s, 2H, NH₂). ¹³C NMR (101 MHz, DMSO-d₆) δ 155.7 (C), 154.3 (C), 146.0*(CH), 143.7 (C), 137.4 (CH), 131.4† (C), 126.1 (q, J=3.7 Hz, CF₃), 125.6 (C), 125.1 (CH), 123.3 (CH), 122.9 (C). LCMS R_{f} (min)=4.91, MS m/z=362.0 [M−H]⁻. HRMS (ESI) calcd for C₁₆H₁₃F₃N₅O₂⁺ [M+H]⁺ 364.1016, found 364.1012.
* Missing resonance: CH signal is below the signal to noise ratio by ¹³C NMR, found using HSQC
† Overlapping quaternary carbon resonances
‡ Overlapping CH resonances (—CF₃ substituted ring)

### 4. N-(6-(Hydroxyamino)pyridin-3-yl)-5-(4-(trifluoromethyl)phenyl)oxazol-2-amine (Scheme 3)

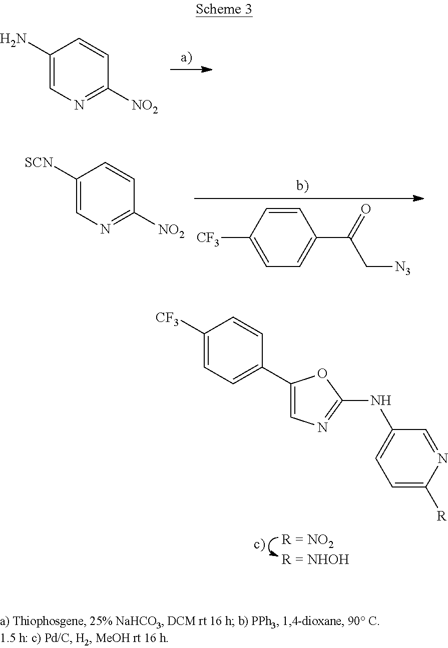

To a suspension of 6-nitropyridin-3-amine (0.050 g, 0.36 mmol) in DCM (1.5 mL) and 25% NaHCO₃ (aq.) (1.5 mL) was added neat thiophosgene (0.030 mL, 0.40 mmol) at 0° C. and the mixture stirred at rt for 16 h. After this time, the mixture was diluted with DCM (30 mL) and washed with H₂O (2×15 mL). The organic layer was dried over MgSO₄ and concentrated to give 5-isothiocyanato-2-nitropyridine as a cream solid (0.051 g, 79% yield). ¹H NMR (401 MHz, CDCl₃) δ 3.73 (d, J=2.4 Hz, 1H), 3.54 (dd, J=8.6, 0.5 Hz, 1H), 3.03 (dd, J=8.6, 2.5 Hz, 1H). ¹³C NMR (101 MHz, CDCl₃) δ 153.4 (C), 145.8 (CH), 143.4 (C), 135.6 (CH), 135.5 (C), 119.3 (CH). LCMS R_{f} (min)=5.43, MS m/z=180.1 [M+H]⁺.

A solution of 2-azido-1-(4-(trifluoromethyl)phenyl)ethan-1-one (Intermediate A) (0.425 g, 1.86 mmol), 5-isothiocyanato-2-nitropyridine (0.336 g, 1.86 mmol) and PPh₃ (0.486 g, 1.86 mmol) in 1,4-dioxane was heated to 90° C. for 1.5 h. On cooling, the mixture was concentrated to a dark brown solid that was triturated with DCM (5 mL), affording N-(6-nitropyridin-3-yl)-5-(4-(trifluoromethyl)phenyl)oxazol-2-amine as a brown solid (0.432 g, 67% yield). Mp 319-324° C. ¹H NMR (401 MHz, DMSO-d₆) δ 11.62 (s, 1H, NH), 8.74 (d, J=2.5 Hz, 1H), 8.47 (dd, J=9.0, 2.7 Hz, 1H), 8.39 (d, J=8.9 Hz, 1H), 7.84 (s, 1H), 7.83 (s, 4H). ¹³C NMR (101 MHz, DMSO-d₆) δ 155.7 (C), 149.9 (C), 143.9 (C), 141.1 (C), 136.7 (CH), 131.3 (C), 127.7 (C), 126.2 (q, J=3.8 Hz, CF₃), 125.2 (CH), 124.9 (CH), 123.4*(CH), 120.0 (CH). LCMS R_{f} (min)=3.96, MS m/z=350.8 [M+H]⁺. HRMS (ESI) calcd for C₁₅H₁₀F₃N₄O₃⁺ [M+H]⁺ 351.0700, found 351.0703.
* Overlapping resonances (—CF₃ substituted ring)

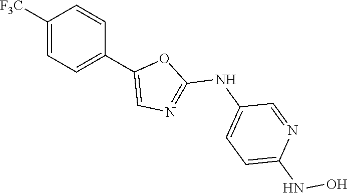 [SMILES: ONc1ccc(Nc2ncc(-c3ccc(C(F)(F)F)cc3)o2)cn1]

Pd/C 10% (0.025 g) was added to a solution of N-(6-nitropyridin-3-yl)-5-(4-(trifluoromethyl)phenyl)oxazol-2-amine (0.040 g, 0.11 mmol) in MeOH (1.5 mL) and the mixture placed under vacuum and flushed with H₂×3 to remove any oxygen. The reaction mixture was then stirred at rt under H₂ gas for 16 h. After this time, the solution was filtered through celite to remove Pd/C and concentrated to a light brown solid (0.028 g) that was triturated with EtOAc (4 mL), providing the title compound as a cream solid (0.011 g, 29% yield). Mp 212-215° C. ¹H NMR (401 MHz, DMSO-d₆) δ 10.26 (s, 1H, NH), 8.51 (d, J=2.0 Hz, 1H, NH), 8.42 (d, J=1.8 Hz, 1H, OH), 8.34 (d, J=2.3 Hz, 1H), 7.92 (dd, J=8.9, 2.7 Hz, 1H), 7.76 (ABq, J=8.7 Hz, 4H), 7.66 (s, 1H), 6.90 (d, J=8.9 Hz, 1H). ¹³C NMR (101 MHz, DMSO-d₆) δ 158.9 (C), 157.7 (C), 142.4 (C), 136.8 (CH), 131.8 (C), 128.9 (C), 127.6 (CH), 126.0 (q, J=3.9 Hz, CF₃), 125.6 (CH), 122.9 (C), 122.6*(CH), 107.4 (CH). LCMS R_{f} (min)=4.89, MS m/z=336.8 [M+H]⁺. HRMS (ESI) calcd for C₁₅H₁₂F₃N₄O₂⁺ [M+H]⁺ 337.0907, found 337.0905.
* Overlapping resonances (—CF₃ substituted ring)

### 5. 5-((5-[4-(Trifluoromethyl)phenyl]-1,3,4-oxadiazol-2-yl)amino)-N-hydroxy-pyridine-2-carboximidamide (Scheme 4, X═H)

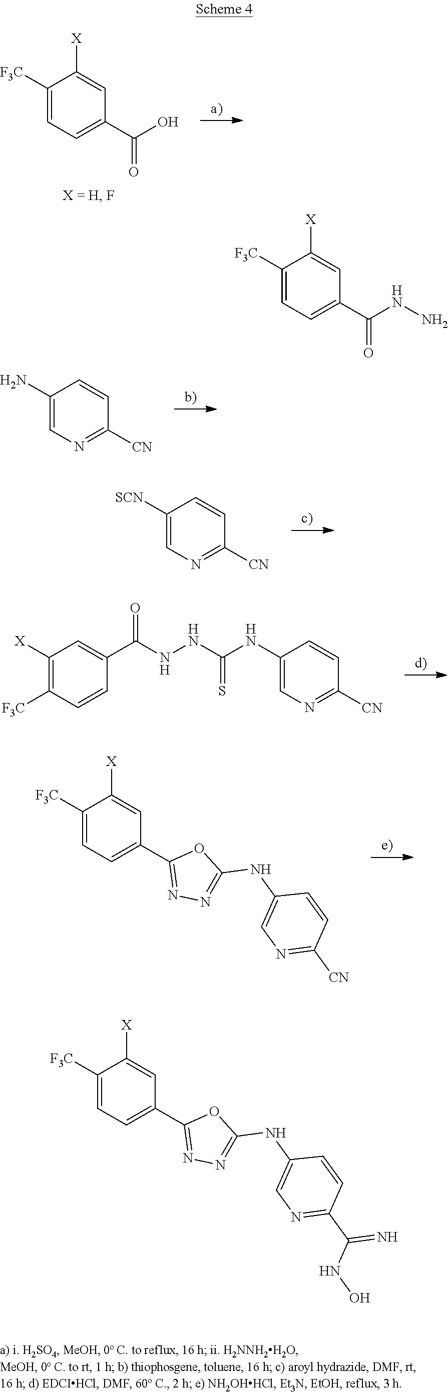

H₂SO₄ (3 mL) was added dropwise to 4-(trifluoromethyl)benzoic acid (10.000 g, 52.598 mmol) in MeOH (250 mL) at 0° C. and the mixture refluxed for 16 h. On cooling, hydrazine monohydrate (73.700 mL) was added and the mixture stirred at rt for 1 h. The solution was then concentrated under reduced pressure. Ice water was added and the resulting precipitate was filtered and washed with H₂O (10 mL) and Et₂O (3 mL), giving 4-(trifluoromethyl)benzohydrazide as a white crystalline solid (0.847 g, 78.9% yield). ¹H NMR (400 MHz, DMSO-d₆) δ 9.90 (s, 1H), 7.94 (d, J=8.3 Hz, 2H), 7.45 (d, J=8.2 Hz, 2H), 4.68 (s, 2H). ¹³C NMR (101 MHz, DMSO-d₆) δ 164.6, 150.1, 132.4, 129.2, 121.2, 120.6. LCMS R_{f} (min)=5.29, MS m/z=220.1 [M+H]⁺.

Thiophosgene (1.1 equiv.) was added to a suspension of 5-aminonicotinonitrile (1.000 g, 8.394 mmol) in toluene (40 mL) and the mixture was heated at reflux for 2 h. Volatiles were then removed in vacuo and the residue redissolved in dry DMF (6 mL). To the solution was then added 4-(trifluoromethyl)benzohydrazide (1 equiv.) and the mixture stirred at rt for 16 h. EDCI·HCl (1.2 equiv.) was then added and the mixture heated at 60° C. for 2 h. On cooling, H₂O (5 mL) was added and the mixture stirred at rt for 0.5 h. The resulting precipitate was filtered and washed with H₂O (5 mL) and DCM (2 mL), providing 5-((5-(4-(trifluoromethyl)phenyl)-1,3,4-oxadiazol-2-yl)amino)picolinonitrile as a yellow solid (1.165 g, 86.1% yield). ¹H NMR (400 MHz, DMSO-d₆) δ 11.75 (s, 1H), 8.85 (d, J=2.2 Hz, 1H), 8.29 (dd, J=8.6, 2.4 Hz, 1H), 8.12 (d, J=8.1 Hz, 2H), 8.06 (d, J=8.6 Hz, 1H), 7.97 (d, J=8.2 Hz, 2H). ¹³C NMR (101 MHz, DMSO-d₆) δ 159.93, 158.24, 140.94, 139.00, 130.40, 127.70, 127.07, 126.93, 125.08, 124.09, 118.32. LCMS R_{f} (min)=3.381, MS m/z=331.8 [M+H]⁺.

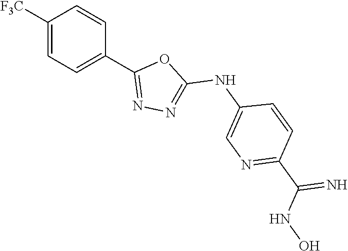 [SMILES: N=C(NO)c1ccc(Nc2nnc(-c3ccc(C(F)(F)F)cc3)o2)cn1]

A solution of 5-((5-(4-(trifluoromethyl)phenyl)-1,3,4-oxadiazol-2-yl)amino)picolinonitrile (0.076 g, 0.229 mmol), NH₂OH (2.5 equiv.) and Et₃N (2.5 equiv.) in EtOH (2 mL) was heated to reflux for 4 h. On reaction completion, the crude material was purified using preparative HPLC in a 95% A: 5% B to 100% B solvent system. The TFA and ACN were removed through rotary evaporation and H₂O through the use of the freeze dryer, providing the title compound as a white solid (0.036 g, 43.1% yield). ¹H NMR (400 MHz, DMSO-d₆) δ 10.88 (s, 1H), 10.20 (s, 1H), 8.62 (s, 2H), 8.16 (d, J=9.1 Hz, 1H), 8.12 (d, J=8.1 Hz, 2H), 7.99 (d, J=8.4 Hz, 2H), 7.63 (d, J=7.5 Hz, 2H. ¹³C NMR (101 MHz, DMSO-d₆) δ 165.77, 164.00, 159.82, 159.69, 157.60, 143.84, 138.49, 137.54, 137.44, 127.29, 126.67, 126.52, 126.45, 125.17, 125.17, 124.22, 122.80, 122.46. LCMS R_{f} (min)=4.926, MS m/z=364.8 [M+H]⁺. HRMS (ESI) calcd for C₁₅H₁₂F₃N₆O₂⁺ [M+H]⁺ 365.097, found 365.0968.

### 6. 5-((5-[3-Fluoro-4-(trifluoromethyl)phenyl]-1,3,4-oxadiazol-2-yl)amino)-N-hydroxypyridine-2-carboximidamide (Scheme 4, X═F)

H₂SO₄ (0.270 mL) was added dropwise to 3-fluoro-4-(trifluoromethyl)benzoic acid (1.000 g, 4.805 mmol) in MeOH (25 mL) at 0° C. and the mixture was refluxed for 16 h. On cooling, hydrazine monohydrate (6.730 mL) was added and the mixture stirred at rt for 1 h. The solution was then concentrated under reduced pressure. Ice water was added and the resulting precipitate was filtered and washed with H₂O (10 mL) and Et₂O (3 mL), giving 3-fluoro-4-(trifluoromethyl)benzohydrazide as a white crystalline solid (0.869 g, 81.4% yield). ¹H NMR (400 MHz, CDCl₃) δ 7.74 (t, J=7.4 Hz, 1H), 7.63 (t, J=10.1 Hz, 2H), 7.40 (s, 1H), 4.15 (s, 2H). ¹³C NMR (101 MHz, DMSO-d₆) δ 164.6, 150.1, 132.4, 129.2, 121.2, 120.6. LCMS R_{f} (min)=3.003, MS m/z=222.9 [M+H]⁺.

Thiophosgene (1.1 equiv.) was added to a suspension of 5-amino-2-pyridinecarbonitrile (0.134 g, 1.125 mmol) in toluene (8 mL) and the mixture was heated to reflux for 2 h. Volatiles were then removed in vacuo and the residue redissolved in dry DMF (6 mL). To the solution was then added 3-fluoro-4-(trifluoromethyl)benzohydrazide (1 equiv.) and the mixture stirred at rt for 16 h. EDCI·HCl (1.2 equiv.) was then added and the mixture heated at 60° C. for 2 h. On cooling, H₂O (5 mL) was added and the mixture stirred at rt for 0.5 h. The resulting precipitate was filtered and washed with H₂O (5 mL) and DCM (1 mL), providing 5-((5-(3-fluoro-4-(trifluoromethyl)phenyl)-1,3,4-oxadiazol-2-yl)amino)picolinonitrile as a yellow solid (0.277 g, 70.5% yield). ¹H NMR (400 MHz, DMSO-d₆) δ 8.86 (s, 1H), 8.30 (d, J=8.4 Hz, 1H), 8.05 (dd, J=15.0, 8.0 Hz, 2H), 7.96 (dd, J=15.6, 9.9 Hz, 2H). ¹³C NMR (101 MHz, DMSO-d₆) δ 174.82, 160.05, 140.90, 138.76, 130.27, 130.06, 129.35, 125.17, 124.06, 122.69, 118.22, 114.62, 114.40. LCMS R_{f} (min)=3.523, MS m/z=349.8 [M+H]⁺.

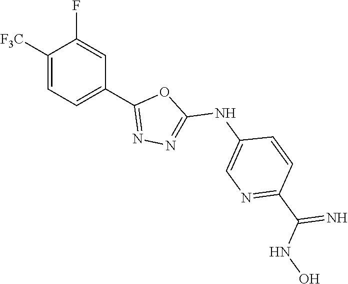 [SMILES: N=C(NO)c1ccc(Nc2nnc(-c3ccc(C(F)(F)F)c(F)c3)o2)cn1]

A solution of 5-((5-(3-fluoro-4-(trifluoromethyl)phenyl)-1,3,4-oxadiazol-2-yl)amino)picolinonitrile (0.100 g, 0.286 mmol), NH₂OH·HCl (2.5 equiv.) and Et₃N (2.5 equiv.) in EtOH (2 mL) was heated to reflux for 4 h. Upon reaction completion, H₂O (50 mL) was added and the solution was extracted with EtOAc (3×15 mL). The combined organic layers were dried over MgSO₄ and concentrated to give the title compound as a white solid (0.028 g, 26.9% yield). ¹H NMR (400 MHz, DMSO-d₆) δ 11.30 (s, 1H), 9.80 (s, 1H), 8.80 (d, J=2.5 Hz, 1H), 8.10 (dd, J=8.8, 2.6 Hz, 1H), 8.04 (t, J=7.7 Hz, 1H), 7.93 (m, 3H), 5.80 (s, 2H). ¹³C NMR (101 MHz, DMSO-d₆) δ 160.19, 157.77, 156.38, 149.26, 143.85, 137.19, 135.44, 129.84, 128.86, 124.62, 123.62, 122.11, 120.92, 119.84, 114.10, 113.87. LCMS R_{f} (min)=5.319. HRMS (ESI) calcd for C₁₅H₁₁F₄N₆O₂⁺ [M+H]⁺ 383.0874, found 383.0875.

### 7. 1-Hydroxy-4-((5-[4-(trifluoromethyl)phenyl]-1,3,4-oxadiazol-2-yl)amino)-1,2-dihydropyridin-2-one (Scheme 5)

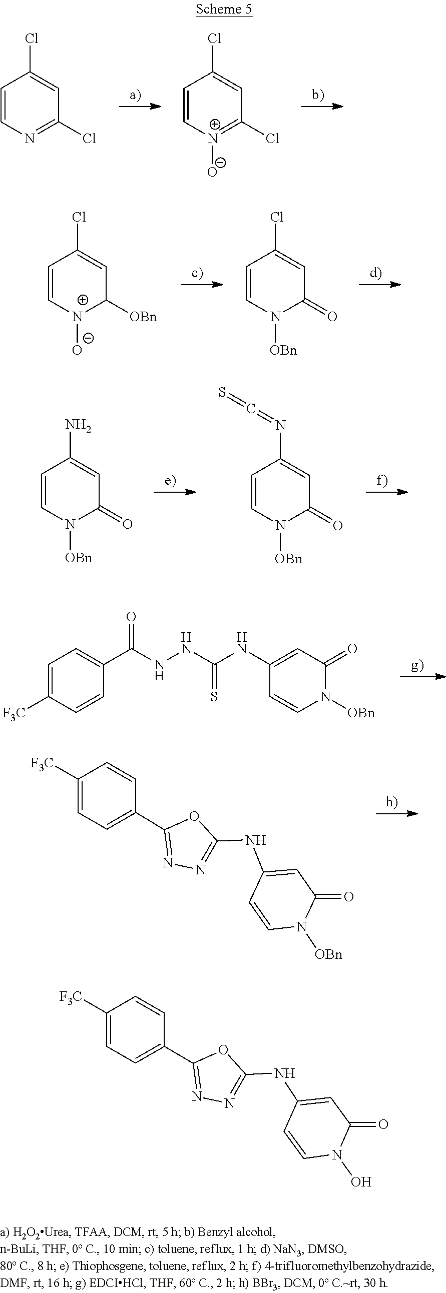

H₂O₂·urea (2 equiv.) was added to a solution of 2,4-dichloropyridine (2.000 g, 13.514 mmol) in dry DCM (60 mL). On cooling to 0° C., a solution of TFAA (2 equiv.) in dry DCM was added dropwise and the mixture was stirred at rt for 5 h. The reaction mixture was diluted with sat. NaS₂O₃ (15 mL) and stirred at rt for 0.5 h then poured into H₂O (10 mL) and extracted with DCM (3×10 mL). The combined organic layers were washed with 1 M NaOH (15 mL), dried over MgSO₄ and concentrated to a cream semi-solid. The solid was then chromatographed on silica gel eluting with 5% EtOH in DCM, providing a yellow crystalline solid (2.068 g, 93.3% yield). ¹H NMR (400 MHz, DMSO-d₆) δ 8.29-8.23 (m, 1H), 7.51 (d, J=2.9 Hz, 1H), 7.20 (dd, J=7.1, 2.9 Hz, 1H). ¹³C NMR (101 MHz, DMSO-d₆) δ 142.9, 140.9, 131.4, 127.0, 124.5. LCMS R_{f} (min)=1.88. MS m/z 164.0 [M+H]⁺.

n-BuLi 2.37 M in cyclohexane (1.1 equiv.) was added dropwise to a solution of benzyl alcohol (1.5 equiv.) in dry THF (10 mL) at 0° C. under N₂ and the mixture was stirred at this temperature for 10 min. The mixture was transferred dropwise into a solution of 2,4-dichloropyridine-1-oxide (0.448 g, 2.732 mmol) in dry THF (10 mL) and the mixture stirred at 0° C. for 15 min. The reaction mixture was quenched with H₂O (10 mL) and extracted with EtOAc (2×20 mL). The combined organic layers were washed with H₂O (2×10 mL), dried over MgSO₄ and concentrated to an orange cream semi-solid. The solid was chromatographed on silica gel eluting with 20% EtOAc in DCM, providing a white crystalline solid (1.282 g, 88.4% yield), which should be stored at −20° C. ¹H NMR (400 MHz, CDCl₃) δ 8.15 (d, J=6.9 Hz, 1H), 7.43 (dd, J=7.9, 1.5 Hz, 2H), 7.40-7.30 (m, 3H), 6.88 (dd, J=6.9, 2.7 Hz, 1H), 6.85 (d, J=2.7 Hz, 1H), 5.39 (s, 2H). ¹³C NMR (101 MHz, DMSO-d₆) δ 157.87, 140.34, 134.09, 132.80, 128.99, 128.96, 127.75, 118.71, 112.02, 72.82. LCMS R_{f} (min)=2.975, MS m/z=235.9 [M+H]⁺.

The solution of 2-(benzyloxy)-4-chloropyridine-1-oxide (1.282 g, 5.440 mmol) in toluene (10 mL) was heated at 100° C. for 3 h. The solvent was then removed through rotary evaporation. The residual solid was chromatographed on silica gel eluting with 10% EtOAc in DCM, providing 1-(benzyloxy)-4-chloropyridin-2(1H)-one as a yellow crystalline solid (0.620 g, 48.3% yield). ¹H NMR (400 MHz, CDCl₃) δ 7.38 (m, 5H), 7.01 (d, J=7.5 Hz, 1H), 6.70 (d, J=2.6 Hz, 1H), 5.92 (dd, J=7.6, 2.7 Hz, 1H), 5.25 (s, 2H). ¹³C NMR (101 MHz, DMSO) δ 157.66, 145.80, 136.66, 133.36, 130.16, 129.61, 128.90, 121.10, 106.10, 78.65. LCMS R_{f} (min)=3.222, MS m/z=257.8 [M+Na]⁺.

NaN₃ (3 equiv.) was added to a solution of 1-(benzyloxy)-4-chloropyridin-2-one (0.500 g, 2.122 mmol) in DMSO (10 mL). The solution was then heated at 80° C. for 28 h. The reaction mixture was cooled to rt and diluted with 1N HCl (2 mL). The resulting mixture was poured into distilled water (20 mL) and the aq. layer was washed with EtOAc (2×20 mL). The aq. layer was slowly neutralized with sat. aq. NaHCO₃ solution and extracted with EtOAc (2×20 mL). The combined organic layers were collected, dried over MgSO₄ and concentrated to a cream semi-solid. The solid was chromatographed on silica gel eluting with 1% Et₃N in EtOAc, providing 4-amino-1-(benzyloxy)pyridin-2(1H)-one as a yellow crystalline solid (0.220 g, 42.8% yield). ¹H NMR (400 MHz, CDCl₃) δ 7.42-7.30 (m, 5H), 6.83 (d, J=7.6 Hz, 1H), 5.69 (d, J=2.3 Hz, 1H), 5.41 (dd, J=7.6, 2.5 Hz, 1H), 5.17 (s, 2H), 4.52 (s, 2H). ¹³C NMR (101 MHz, CDCl₃) δ 159.97, 155.23, 136.24, 134.18, 130.09, 129.16, 128.69, 97.51, 97.00, 78.58, 40.96. LCMS R_{f} (min)=2.920, MS m/z=216.9 [M+H]⁺.

Thiophosgene (1.2 equiv.) was added dropwise to a solution of 1-benzyloxy-4-amino-2-pyridin-2-one (0.200 mg, 0.925 mmol) in dry toluene (5 mL) at rt. The solution was then heated at reflux for 2 h. Reaction progress was monitored by ¹H NMR. After 2 h the starting material had completely disappeared and a new product was formed as confirmed by ¹H NMR. Toluene and thiophosgene were removed in vacuo and the residue redissolved in dry DMF (5 mL). To the solution was then added 4-(trifluoromethyl)benzohydrazide (1 equiv.) and the mixture stirred at rt for 16 h. EDCI·HCl (1.2 equiv.) was then added and the mixture heated to 60° C. for 2 h. On cooling, H₂O (5 mL) was added and the mixture stirred at rt for 0.5 h. The resulting precipitate was filtered and washed with H₂O (5 mL), providing 1-(benzyloxy)-4-((5-(4-(trifluoromethyl)phenyl)-1,3,4-oxadiazol-2-yl)amino)pyridin-2(1H)-one as a brown solid (0.176 g, 42.9% yield). ¹H NMR (400 MHz, DMSO-d₆) δ 11.24 (s, 1H), 8.11 (d, J=8.1 Hz, 2H), 7.97 (d, J=8.3 Hz, 2H), 7.72 (d, J=7.8 Hz, 1H), 7.49 (dd, J=6.6, 3.1 Hz, 2H), 7.44-7.40 (m, 3H), 6.91 (d, J=2.9 Hz, 1H), 6.23 (dd, J=7.8, 2.9 Hz, 1H), 5.19 (s, 2H). LCMS R_{f} (min)=3.359, MS m/z=428.8 [M+H]⁺.

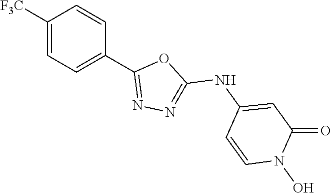 [SMILES: O=c1cc(Nc2nnc(-c3ccc(C(F)(F)F)cc3)o2)ccn1O]

BBr₃ (10 equiv.) was added dropwise to a solution of 1-(benzyloxy)-4-((5-(4-(trifluoromethyl)phenyl)-1,3,4-oxadiazol-2-yl)amino)pyridin-2(1H)-one (0.080 mg, 0.411 mmol) in dry DCM (4 mL) under N₂ at 0° C. The reaction was stirred at rt for 30 h. Sat. NaHCO₃ solution was then poured into the flask to quench BBr₃ and the mixture was stirred for 2 h. The crude material was purified using preparative HPLC in a 95% A: 5% B to 100% B solvent system. The TFA and ACN were removed through rotary evaporation and H₂O through freeze-drying, providing the title compound as a white solid (0.015 g, 29.0% yield). ¹H NMR (400 MHz, DMSO-d₆) δ 11.17 (s, 1H), 8.11 (d, J=8.1 Hz, 2H), 7.97 (d, J=8.3 Hz, 2H), 7.85 (d, J=7.7 Hz, 1H), 6.87 (d, J=2.8 Hz, 1H), 6.33 (dd, J=7.7, 2.9 Hz, 1H). LCMS R_{f} (min)=6.009. HRMS (ESI) calcd for C₁₄H₁₀F₃N₄O₃⁺ [M+H]⁺ 339.0700, found 339.0697.

### 8. 5-((5-[4-(Trifluoromethyl)phenyl]-1,3-oxazol-2-yl)amino)-N-hydroxy-pyridine-2-carboximidamide (Scheme 6)

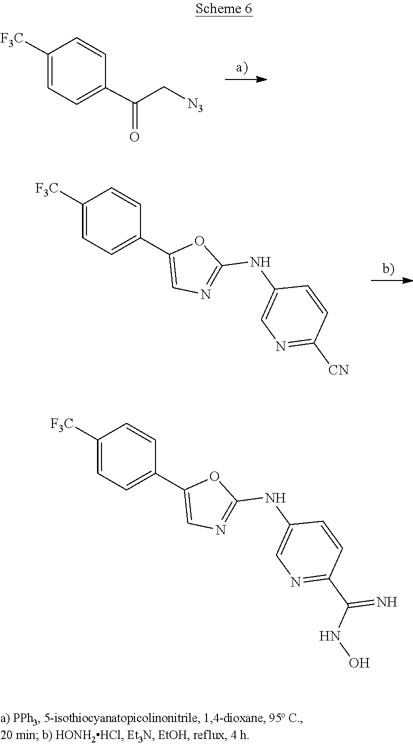 [SMILES: [N-]=[N+]=NCC(=O)c1ccc(C(F)(F)F)cc1 ; N#Cc1ccc(Nc2ncc(-c3ccc(C(F)(F)F)cc3)o2)cn1 ; N=C(NO)c1ccc(Nc2ncc(-c3ccc(C(F)(F)F)cc3)o2)cn1]

### Intermediate C—5-((5-(4-(trifluoromethyl)phenyl)oxazol-2-yl)amino)picolinonitrile

To the solution of 2-azido-1-(4-(trifluoromethyl)phenyl)ethan-1-one in dry 1,4-dioxane (Intermediate A) (10 mL) was added 5-isothiocyanatopicolinonitrile (1.15 equiv.) and PPh₃ (1.15 equiv.) and the mixture heated at 95° C. for 20 min. Upon completion, the reaction mixture was concentrated under reduced pressure. The crude residue was chromatographed on silica gel eluting with 30% EtOAc in toluene to give 5-((5-(4-(trifluoromethyl)phenyl)oxazol-2-yl)amino)picolinonitrile as a yellow crystalline solid (0.380 g, 66.9% yield). ¹H NMR (400 MHz, DMSO-d₆) δ 11.42 (s, 1H), 8.86 (d, J=2.2 Hz, 1H), 8.34 (dd, J=8.7, 2.7 Hz, 1H), 8.01 (d, J=8.6 Hz, 1H), 7.83 (br s, 4H), 7.82 (s, 1H). ¹³C NMR (101 MHz, DMSO-d₆) δ 164.6, 150.1, 132.4, 129.2, 121.2, 120.6. LCMS R_{f} (min)=6.079, MS m/z=329.1 [M−H]⁻.

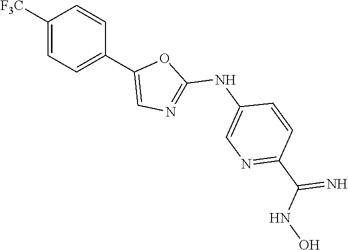 [SMILES: N=C(NO)c1ccc(Nc2ncc(-c3ccc(C(F)(F)F)cc3)o2)cn1]

Et₃N (2.5 equiv.) was added to a solution of 5-((5-(4-(trifluoromethyl)phenyl)oxazol-2-yl)amino)picolinonitrile (0.080 g, 0.242 mmol) and NH₂OH·HCl (2.5 equiv.) in EtOH (10 mL) and the mixture heated at reflux for 4 h. On reaction completion, the solution was diluted with EtOAc (30 mL) and washed with H₂O (3×15 mL). The aq. layers were then collected and back extracted with EtOAc (3×15 mL). The combined organic layers were dried over MgSO₄ and concentrated to a yellow solid. The crude product was then redissolved and recrystallized in EtOH, providing the title compound as a yellow crystalline solid (0.036 g, 40.9% yield). ¹H NMR (400 MHz, DMSO-d₆) δ 10.89 (s, 1H), 9.74 (s, 1H), 8.78 (d, J=2.2 Hz, 1H), 8.13 (dd, J=8.8, 2.6 Hz, 1H), 7.84 (d, J=8.7 Hz, 1H), 7.80 (s, 4H), 7.76 (s, 1H), 5.76 (s, 2H). LCMS R_{f} (min)=5.935. HRMS (ESI) calcd for C₁₆H₁₃F₃N₅O₂⁺ [M+H]⁺ 364.1025, found 364.1016.

### 9. 5-([4-(4-Chlorophenyl)-1,3-thiazol-2-yl]amino)-N-hydroxypyrimidine-2-carboxamide (Scheme 7)

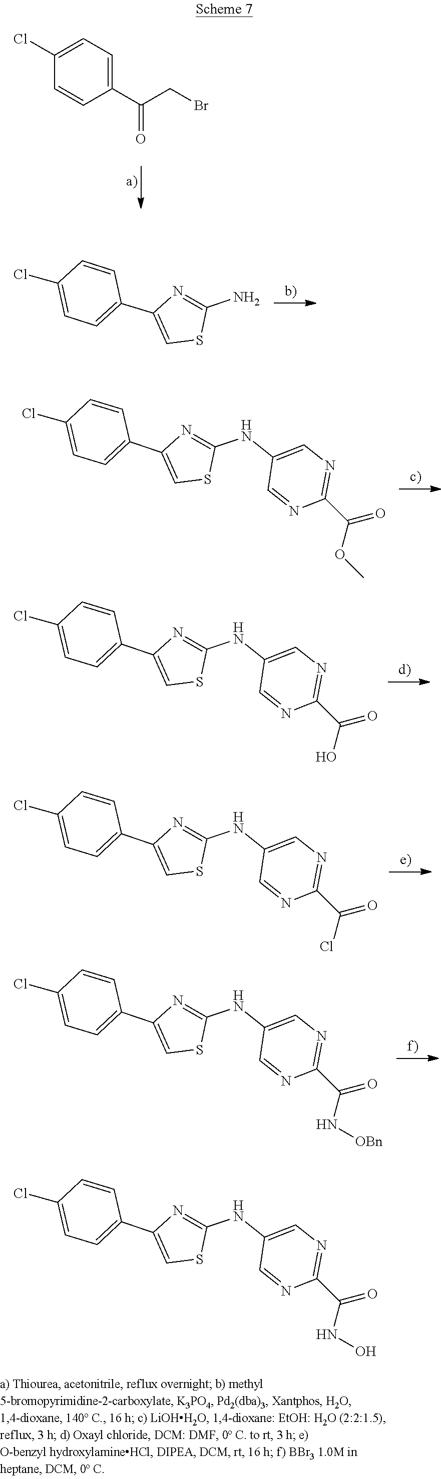

Thiourea (6.841 g, 89.871 mmol) was added to solution of 2-bromo-1-(4-chlorophenyl)ethan-1-one (2.000 g, 7.489 mmol) in acetonitrile (50 mL) and the mixture heated to reflux for 16 h. On cooling, the mixture was concentrated under vacuum. The mixture was then washed with H₂O (10 mL) and DCM (5 mL) to afford 4-(4-chlorophenyl)thiazol-2-amine as a pale yellow solid. ¹H NMR (401 MHz, DMSO-d₆) δ 7.83-7.77 (m, 2H), 7.45-7.36 (m, 2H), 7.08 (s, 2H), 7.03 (s, 1H). ¹³C NMR (101 MHz, DMSO-d₆) δ 168.44, 148.65, 133.78, 131.62, 128.53, 127.28, 102.38. LCMS R_{f} (min)=3.112, MS m/z=210.9 [M+H]⁺.

A re-sealable Schlenk tube was charged with Pd₂(dba)₃ (0.02 equiv.), Xantphos (0.06 equiv.), 5-(4-chlorophenyl)thiazol-2-amine (1.2 equiv.), K₃PO₄ (fine powder, 1.4 equiv.), methyl 5-bromopyrimidine-2-carboxylate (0.150 g, 0.691 mmol) and 1,4-dioxane (4 mL). After the mixture was degassed and carefully subjected to three cycles of evacuation and backfilling with N₂, H₂O (1.0 mmol) was added dropwise. This was then sealed and immersed in a 140° C. oil bath. After 15 h, the mixture was cooled and filtered. The precipitate was washed with H₂O (5 mL) and DCM (2 mL), providing methyl 5-((4-(4-chlorophenyl)thiazol-2-yl)amino)pyrimidine-2-carboxylate as a grey solid (0.13 g, 54.2% yield). ¹H NMR (401 MHz, DMSO) δ 9.32 (s, 2H), 8.18-7.91 (m, 2H), 7.61 (s, 1H), 7.53-7.43 (m, 2H), 3.88 (s, 3H). ¹³C NMR (101 MHz, DMSO-d₆) δ 163.19, 155.43, 148.43, 144.37, 144.35, 137.93, 135.93, 134.84, 128.11, 127.79, 125.72, 125.57, 122.90, 52.49. LCMS R_{f} (min)=3.646, MS m/z=344.8 [M−H]⁻.

LiOH·H₂O (3 equiv.) in H₂O (1.5 mL) was added to a solution of methyl 5-((4-(4-chlorophenyl)thiazol-2-yl)amino)pyrimidine-2-carboxylate (0.200 g, 0.577 mmol) in 1,4-dioxane (2 mL) and EtOH (2 mL) and the mixture refluxed for 3 h. Volatiles were removed in vacuo and to the suspension was added brine (3 mL), followed by 6 M HCl (2 mL) dropwise at 0° C. The resulting precipitate was filtered and washed with H₂O (3 mL), providing 5-((4-(4-chlorophenyl)thiazol-2-yl)amino)pyrimidine-2-carboxylic acid as a yellow solid (0.180 g, 93% yield). ¹H NMR (400 MHz, DMSO-d₆) δ 11.14 (s, 1H), 9.32 (s, 2H), 7.99 (d, J=8.4 Hz, 2H), 7.63 (s, 1H), 7.52 (d, J=8.4 Hz, 2H). ¹³C NMR (101 MHz, DMSO-d₆) δ 164.70, 162.20, 149.48, 144.77, 137.53, 133.34, 132.79, 129.29, 127.95, 106.81.

Oxalyl chloride (2.5 equiv.) was added dropwise to a solution of 5-((4-(4-chlorophenyl)thiazol-2-yl)amino)pyrimidine-2-carboxylic acid (0.100 g, 0.301 mmol) in dry DCM (2 mL) and dry DMF (1 drop) at 0° C. and the mixture stirred at rt for 3 h. The solvent was removed in vacuo and the residue redissolved in dry DCM. O-benzylhydroxylamine hydrochloride (5 equiv.) and DIPEA (5 equiv.) were added and the mixture stirred at rt for a further 16 h. Upon completion, all the volatiles were removed under reduced pressure. H₂O (2 mL) was then added. The resulting precipitate was filtered and washed with DCM (2 mL), H₂O (1 mL) and Et₂O (1 mL) to give N-(benzyloxy)-5-((4-(4-chlorophenyl)thiazol-2-yl)amino)pyrimidine-2-carboxamide as a pale yellow solid (0.062 g, 90.4% yield). ¹H NMR (401 MHz, DMSO) δ 12.06 (s, 1H), 11.11 (s, 1H), 9.31 (s, 2H), 7.98 (d, J=8.6 Hz, 2H), 7.64 (s, 1H), 7.54 (d, J=8.6 Hz, 2H), 7.49 (d, J=6.9 Hz, 2H), 7.43-7.36 (m, 3H), 4.95 (s, 2H). LCMS R_{f} (min)=3.646, MS m/z=435.8 [M−H]⁻. The crude material was used in the next step without further purification.

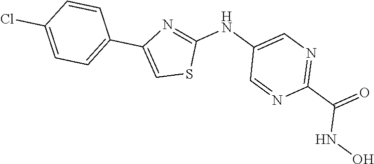 [SMILES: O=C(NO)c1ncc(Nc2nc(-c3ccc(Cl)cc3)cs2)cn1]

BBr₃ 1.0 M in heptane (3 equiv.) was added dropwise to a solution of N-(benzyloxy)-5-([4-(4-chlorophenyl)-1,3-thiazol-2-yl]amino)pyrimidine-2-carboxamide (0.060 g, 0.132 mmol) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The crude material was purified using preparative HPLC in 95% A: 5% B to 100% B solvent system. The TFA and ACN were removed through rotary evaporation and H₂O through freeze-drying, providing the title compound as a white solid (0.015 g, 31.2% yield). ¹H NMR (400 MHz, DMSO-d₆) δ 11.04 (s, 1H), 9.28 (s, 2H), 9.12 (s, 1H), 7.97 (d, J=8.6 Hz, 2H), 7.61 (s, 1H), 7.52 (d, J=8.6 Hz, 2H). LCMS R_{f} (min)=3.236. HRMS (ESI) calcd for C₁₄H₁₁ClN₅O₂S⁺ [M+H]⁺ 348.0311, found 348.0366.

### 10. N-Hydroxy-6-((5-[4-(trifluoromethyl)phenyl]-1,3-oxazol-2-yl)amino)pyrimidine-3-carboxamide (Scheme 8)

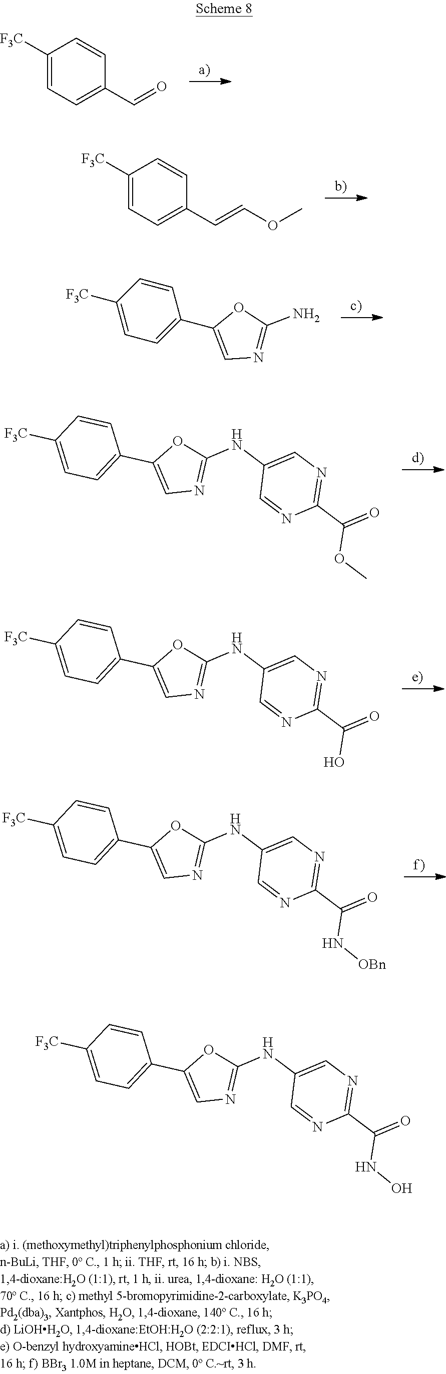

n-BuLi (2.17 M in cyclohexane) (1.2 equiv.) was added dropwise to a solution of (methoxymethyl)triphenylphosphonium chloride (1.2 equiv.) in dry THF (15 mL) at 0° C. and the mixture stirred at this temperature for 1 h. 4-(trifluoromethyl)benzaldehyde (3.930 mL, 28.716 mmol) was then added and the mixture stirred at rt for 16 h. The mixture was quenched with H₂O (3 mL) and extracted with EtOAc (2×20 mL). The combined organic layers were then dried over MgSO₄ and concentrated to yellow liquid. Crude material was chromatographed on silica gel eluting with 100% hexanes providing (E)-1-(2-methoxyvinyl)-4-(trifluoromethyl)benzene as a colourless liquid (5.430 g, 77.9% yield). ¹H NMR (400 MHz, CDCl₃) δ 7.65 (d, J=8.2 Hz, 2H), 7.51 (d, J=8.3 Hz, 2H), 6.24 (d, J=7.0 Hz, 1H), 5.25 (d, J=7.0 Hz, 1H), 3.82 (s, 3H).

### Intermediate D Synthesis—5-(4-(trifluoromethyl)phenyl)oxazol-2-amine

NBS (1.1 equiv.) was added to solution of (E)-1-trifluoromethyl-4-(2-methoxyvinyl)benzene (5.000 g, 24.730 mmol) in H₂O (25 mL) and 1,4-dioxane (25 mL) at 0° C. and the mixture stirred at rt for 1 h. Urea (1 equiv.) was then added and the mixture heated at 70° C. for 16 h. On cooling, volatiles were removed under reduced pressure and the mixture was quenched with sat. NaHCO₃ (aq.). The precipitate was then filtered and washed with H₂O (20 mL) and DCM (10 mL) to afford 5-(4-(trifluoromethyl)phenyl)oxazol-2-amine as a white solid (3.200 g, 56.7% yield). ¹H NMR (401 MHz, CDCl₃) δ 7.71 (d, J=8.4 Hz, 2H), 7.63 (d, J=8.2 Hz, 2H), 7.42 (s, 1H), 7.06 (s, 2H). LCMS R_{f} (min)=3.331, MS m/z=228.9 [M+H]⁺.

A re-sealable Schlenk tube was charged with Pd₂(dba)₃ (0.02 equiv.), Xantphos (0.06 equiv.), 5-(4-(trifluoromethyl)phenyl)oxazol-2-amine (Intermediate D) (1.2 equiv.), K₃PO₄ (fine powder, 1.4 equiv.), methyl 5-bromopyrimidine-2-carboxylate (0.250 g, 1.152 mmol) and 1,4-dioxane (4 mL). After the mixture was degassed and carefully subjected to three cycles of evacuation and backfilling with N₂, H₂O (0.021 g, 1.0 mmol) was added dropwise. This was then sealed and immersed in a 140° C. oil bath. After 15 h, the mixture was cooled, diluted with EtOAc (20 mL) and washed with H₂O (15 mL). The aq. layers were then back extracted with EtOAc (2×15 mL). The crude product was concentrated under reduced pressure and chromatographed on a SiO₂ column (EtOAc:toluene=1:1) to give methyl 5-((5-(4-(trifluoromethyl)phenyl)oxazol-2-yl)amino)pyrimidine-2-carboxylate as a white solid (0.21 g, 50.0% yield). ¹H NMR (400 MHz, DMSO-d₆) δ 9.05 (s, 2H), 7.77 (s, 4H), 7.75 (s, 1H), 3.85 (s, 3H). ¹³C NMR (101 MHz, DMSO-d₆) δ 163.29, 147.65, 144.76, 143.29, 131.51, 126.06, 126.03, 125.43, 123.02, 52.37. LCMS R_{f} (min)=3.523, MS m/z=364.8 [M+H]⁺.

LiOH·H₂O (3 equiv.) in H₂O (1 mL) was added to a solution of methyl 5-((5-(4-(trifluoromethyl)phenyl)oxazol-2-yl)amino)pyrimidine-2-carboxylate (0.125 g, 0.343 mmol) in 1,4-dioxane (1.5 mL) and EtOH (1.5 mL) and the mixture refluxed for 3 h. Volatiles were removed in vacuo and to the suspension was added brine (2 mL), followed by 6 M HCl (2 mL) dropwise at 0° C. The resulting precipitate was filtered and washed with H₂O (2 mL), providing 5-((5-(4-(trifluoromethyl)phenyl)oxazol-2-yl)amino)pyrimidine-2-carboxylic acid as a yellow solid (0.120 g, 99.8% yield). ¹H NMR (400 MHz, DMSO-d₆) δ 11.37 (s, 1H), 9.16 (s, 2H), 7.83 (s, 5H). LCMS R_{f} (min)=3.660. ¹³C NMR (101 MHz, DMSO-d₆) δ 164.21, 155.68, 149.71, 144.45, 143.78, 135.52, 131.30, 127.48, 127.16, 126.11, 126.07, 125.52, 125.21, 123.21, 122.82. MS m/z=350.8 [M+H]⁺.

A solution of 5-((5-(4-(trifluoromethyl)phenyl)oxazol-2-yl)amino)pyrimidine-2-carboxylic acid (0.100 g, 0.286 mmol), anhydrous HOBt (1.2 equiv.) and EDCI·HCl (1.3 equiv.) in dry DMF (2 mL) was stirred at rt for 2 h. O-Benzylhydroxylamine hydrochloride (5 equiv.) and Et₃N (5 equiv.) were then added and the mixture stirred at rt for a further 16 h. DMF was removed in vacuo, washing with toluene (3×2 mL) to aid this process. H₂O (2 mL) was then added and the mixture left to stir at rt for 10 min. The resulting precipitate was filtered and washed with H₂O (1 mL) and hexanes (2 mL), providing N-(benzyloxy)-5-((5-(4-(trifluoromethyl)phenyl)oxazol-2-yl)amino)pyrimidine-2-carboxamide as a brown solid (0.080 g, 61.7% yield). ¹H NMR (401 MHz, DMSO-d₆) δ 12.05 (s, 1H), 9.15 (s, 2H), 7.83 (s, 5H), 7.62-7.20 (m, 5H), 4.94 (s, 2H). LCMS R_{f} (min)=3.551, MS m/z=455.8 [M+H]⁺.

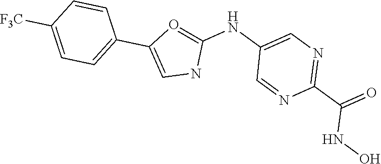

BBr₃ 1.0 M in heptane (3 equiv.) was added dropwise to a solution of N-(benzyloxy)-5-((5-(4-(trifluoromethyl)phenyl)oxazol-2-yl)amino)pyrimidine-2-carboxamide (0.060 g, 0.132 mmol) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The crude material was purified using preparative HPLC in a 95% A: 5% B to 100% B solvent system. The TFA and ACN were removed through rotary evaporation and H₂O through freeze-drying, providing the title compound as a white solid (0.023 g, 47.9% yield). ¹H NMR (400 MHz, DMSO-d₆) δ 11.42 (s, 1H), 9.13 (s, 1H), 9.13 (s, 2H), 7.83 (s, 4H), 7.82 (s, 1H). ¹³C NMR (101 MHz, DMSO-d₆) δ 160.40, 156.27, 151.38, 144.86, 144.15, 135.68, 131.80, 127.60, 126.61, 126.01, 125.76, 123.65, 123.31, 40.63, 40.42. LCMS R_{f} (min)=5.513 HRMS (ESI) calcd for C₁₅H₁₁F₃N₅O₃⁺ [M+H]⁺ 366.0809, found 366.0807.

### 11. N-Hydroxy-6-((5-[4-(trifluoromethyl)phenyl]-1,3-oxazol-2-yl)amino)pyridazine-3-carboxamide (Scheme 9)

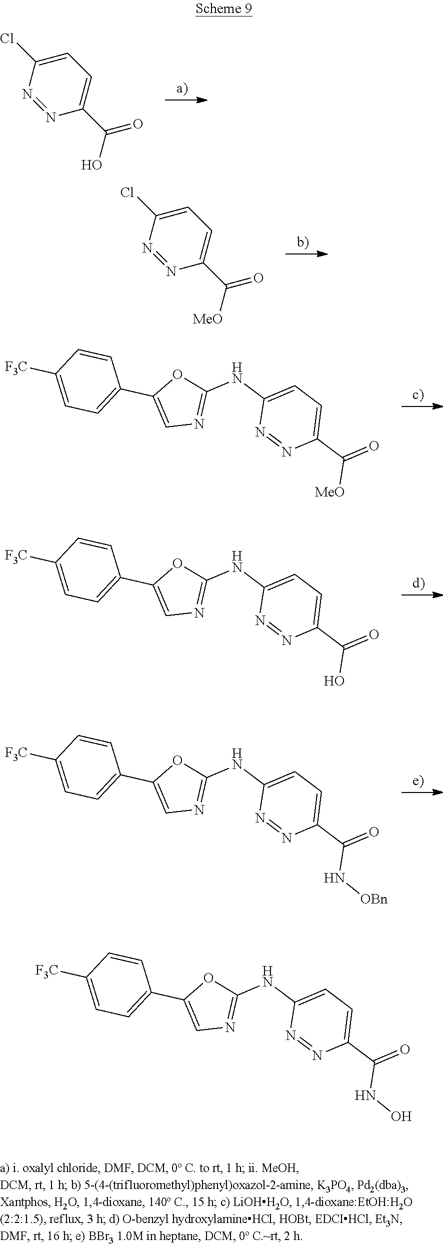

Oxalyl chloride (1 equiv.) was added dropwise to a solution of 6-chloro-3-pyridazinecarboxylic acid (1.000 g, 6.308 mmol) in dry DCM (30 mL) and dry DMF (1 drop) at 0° C. and the mixture stirred at this temperature for 1 h. The solvent was removed in vacuo and the residue redissolved in dry DCM. MeOH (1 equiv.) were added and the mixture stirred at rt for a further 1 h. The mixture was quenched with H₂O (20 mL) and extracted with DCM (2×20 mL). The combined organic layers were then dried over MgSO₄ and concentrated to give methyl 6-chloropyridazine-3-carboxylate as a white solid. (0.960 g, 88.2% yield). ¹H NMR (400 MHz, CDCl₃) δ 8.17 (d, J=8.8 Hz, 1H), 7.68 (d, J=8.8 Hz, 1H), 4.09 (s, 3H).

A re-sealable Schlenk tube was charged with Pd₂(dba)₃ (0.1 equiv.), Xantphos (0.3 equiv.), 5-(4-(trifluoromethyl)phenyl)oxazol-2-amine (Intermediate D) (1.2 equiv.), K₃PO₄ (fine powder, 1.4 equiv.), methyl 6-chloropyridazine-3-carboxylate (0.200 g, 1.159 mmol) and 1,4-dioxane (4 mL). After the mixture was degassed and carefully subjected to three cycles of evacuation and backfilling with N₂, H₂O (1.0 mmol) was added dropwise. This was then sealed and immersed in a 140° C. oil bath. After 15 h, the mixture was cooled, diluted with EtOAc (20 mL) and washed with H₂O (15 mL). The aq. layers were then back extracted with EtOAc (2×15 mL). The crude product was concentrated under reduced pressure and chromatographed on a SiO₂ column (EtOAc:toluene=1:1) to give methyl 6-((5-(4-(trifluoromethyl)phenyl)oxazol-2-yl)amino)pyridazine-3-carboxylate as a white solid (0.28 g, 66.3% yield). ¹H NMR (400 MHz, DMSO-d₆) δ 8.33 (d, J=9.5 Hz, 1H), 8.05 (d, J=9.4 Hz, 1H), 7.79 (s, 4H), 7.75 (s, 1H), 3.90 (s, 3H). LCMS R_{f} (min)=3.907, MS m/z=364.8 [M+H]⁺.

LiOH·H₂O (3 equiv.) in H₂O (1.5 mL) was added to a solution of methyl 6-((5-[4-(trifluoromethyl)phenyl]-1,3-oxazol-2-yl)amino)pyridazine-3-carboxylate (0.140 g, 0.384 mmol) in 1,4-dioxane (2 mL) and EtOH (2 mL) and the mixture refluxed for 3 h. Volatiles were removed in vacuo and to the suspension was added brine (2 mL), followed by 6 M HCl (2 mL) dropwise at 0° C. The resulting precipitate was filtered and washed with H₂O (2 mL), providing 6-((5-(4-(trifluoromethyl)phenyl)oxazol-2-yl)amino)pyridazine-3-carboxylic acid as a yellow solid (0.130 g, 96.6% yield). ¹H NMR (400 MHz, DMSO-d₆) δ 8.44 (s, 1H), 8.20 (s, 1H), 7.84 (s, 5H). LCMS R_{f} (min)=3.797, MS m/z=350.8 [M−H]⁻.

A solution of 6-((5-[4-(trifluoromethyl)phenyl]-1,3-oxazol-2-yl)amino)pyridazine-3-carboxylic acid (0.080 g, 0.228 mmol), anhydrous HOBt (1.1 equiv.) and EDCI·HCl (1.3 equiv.) in dry DMF (2 mL) was stirred at rt for 2 h. After this time, O-benzylhydroxylamine hydrochloride (5 equiv.) and Et₃N (5 equiv.) were added and the mixture stirred at rt for a further 16 h. DMF was removed in vacuo, washing with toluene (2 mL×3) to aid this process. H₂O (2 mL) was then added and the mixture left to stir at rt for 10 min. The resulting precipitate was filtered and washed with H₂O (1 mL) and hexanes (2 mL), providing N-(benzyloxy)-6-((5-(4-(trifluoromethyl)phenyl)oxazol-2-yl)amino)pyridazine-3-carboxamide as a brown solid (0.090 g, 86.5% yield). ¹H NMR (400 MHz, DMSO-d₆) δ 12.35 (s, 1H), 8.49 (s, 1H), 8.18 (s, 2H), 7.85 (s, 4H), 7.60-7.14 (m, 5H), 4.98 (s, 2H). ¹³C NMR (101 MHz, DMSO-d₆) δ 160.71, 144.42, 136.27, 131.78, 129.27, 128.75, 128.29, 128.03, 126.58, 125.99, 125.40, 123.76, 123.29, 77.61. LCMS R_{f} (min)=3.879, MS m/z=455.8 [M+H]⁺.

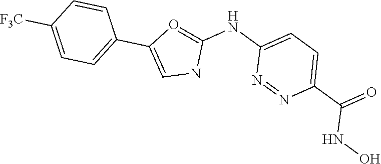

BBr₃ 1.0 M in heptane (3 equiv.) was added dropwise to a solution of N-(benzyloxy)-6-((5-[4-(trifluoromethyl)phenyl]-1,3-oxazol-2-yl)amino)pyridazine-3-carboxamide (0.090 g, 0.198 mmol) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The crude material was purified using preparative HPLC in a 95% A: 5% B to 100% B solvent system. The TFA and ACN were removed through rotary evaporation and H₂O through freeze-drying, providing the title compound as a white solid (0.031 g, 42.9% yield). ¹H NMR (400 MHz, DMSO-d₆) δ 12.09 (s, 1H), 11.69 (s, 1H), 8.48 (s, 1H), 8.14 (d, J=9.0 Hz, 1H), 7.84 (m, 4H), 7.84 (s, 1H). ¹³C NMR (101 MHz, DMSO-d₆) δ 160.59, 156.64, 144.35, 131.79, 128.16, 128.01, 127.69, 126.55, 126.00, 125.34, 123.76, 123.30. LCMS R_{f} (min)=5.501. HRMS (ESI) calcd for C₁₅H₁₁F₃N₅O₃⁺ [M+H]⁺ 366.0811, found 366.0809.

### 12. N-Hydroxy-5-((4-[4-(trifluoromethyl)phenyl]-1,3-oxazol-2-yl)amino)pyrimidine-2-carboxamide (Scheme 10)

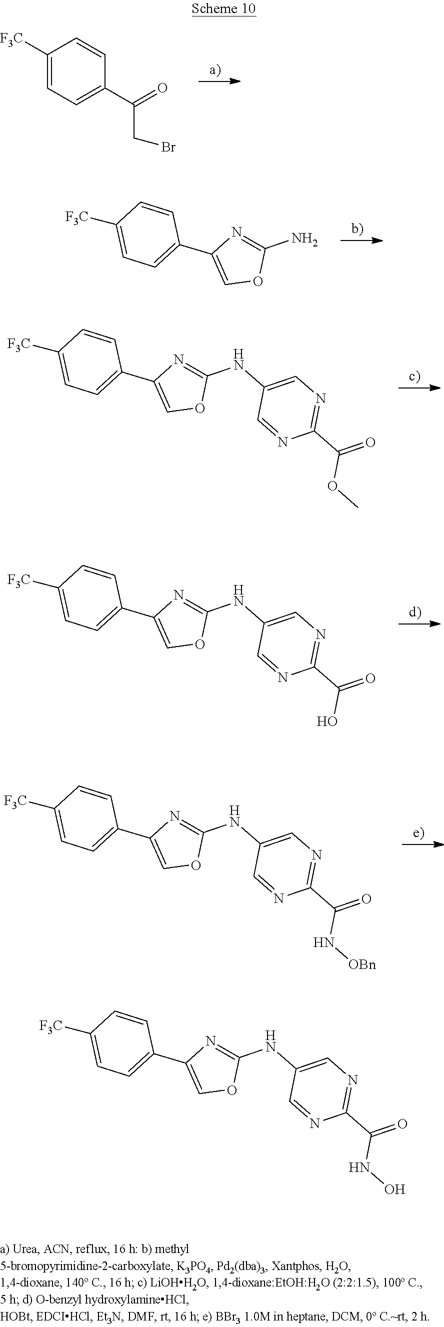 [SMILES: O=C(CBr)c1ccc(C(F)(F)F)cc1 ; Nc1nc(-c2ccc(C(F)(F)F)cc2)co1 ; COC(=O)c1ncc(Nc2nc(-c3ccc(C(F)(F)F)cc3)co2)cn1 ; O=C(O)c1ncc(Nc2nc(-c3ccc(C(F)(F)F)cc3)co2)cn1 ; O=C(NOCc1ccccc1)c1ncc(Nc2nc(-c3ccc(C(F)(F)F)cc3)co2)cn1 ; O=C(NO)c1ncc(Nc2nc(-c3ccc(C(F)(F)F)cc3)co2)cn1]

### Intermediate E Synthesis—4-(4-(trifluoromethyl)phenyl)oxazol-2-amine

Urea (12 equiv.) was added to solution of 2-bromo-1-(4-(trifluoromethyl)phenyl)ethan-1-one (3.000 g, 11.234 mmol) in ACN (30 mL) and the mixture heated to reflux for 16 h. On cooling, the mixture was concentrated under vacuum. The mixture was then filtered with H₂O (10 mL) and washed with DCM (5 mL) to afford 4-(4-(trifluoromethyl)phenyl)oxazol-2-amine as a pale yellow solid (2.200 g, 85.8% yield). ¹H NMR (400 MHz, CDCl₃) δ 7.73 (d, J=8.2 Hz, 2H), 7.63 (d, J=8.3 Hz, 2H), 7.53 (s, 1H), 5.09 (s, 2H). LCMS R_{f} (min)=3.359, MS m/z=228.9 [M+H]⁺.

A re-sealable Schlenk tube was charged with Pd₂(dba)₃ (0.02 equiv.), Xantphos (0.06 equiv.), (4-[4-(trifluoromethyl)phenyl]-1,3-oxazol-2-yl)amine (1.2 equiv.), K₃PO₄ (fine powder, 1.4 equiv.), methyl 5-bromopyrimidine-2-carboxylate (0.150 g, 0.691 mmol) and 1,4-dioxane (4 mL). After the mixture was degassed and carefully subjected to three cycles of evacuation and backfilling with N₂, H₂O (1.0 mmol) was added dropwise. This was then sealed and immersed in a 140° C. oil bath. After 15 h, the mixture was cooled, diluted with EtOAc (20 mL) and washed with H₂O (15 mL). The aq. layers were then back extracted with EtOAc (2×15 mL). The crude product was concentrated under reduced pressure and chromatographed on a SiO₂ column (EtOAc:toluene=1:1) to give methyl 5-((4-(4-(trifluoromethyl)phenyl)oxazol-2-yl)amino)pyrimidine-2-carboxylate as a white solid (0.18 g, 71.5% yield). ¹H NMR (400 MHz, DMSO-d₆) δ 11.29 (s, 1H), 9.26 (s, 2H), 8.49 (s, 1H), 8.04 (d, J=8.1 Hz, 2H), 7.81 (d, J=8.2 Hz, 2H), 3.89 (s, 3H). ¹³C NMR (101 MHz, DMSO-d₆) δ 163.19, 155.43, 148.43, 144.37, 144.35, 137.93, 135.93, 134.84, 128.11, 127.79, 125.72, 125.57, 122.90, 52.49. LCMS R_{f} (min)=3.517, MS m/z=364.8 [M+H]⁺.

LiOH·H₂O (3 equiv.) in H₂O (1 mL) was added to a solution of methyl 5-((4-(4-(trifluoromethyl)phenyl)oxazol-2-yl)amino)pyrimidine-2-carboxylate (0.160 g, 0.439 mmol) in 1,4-dioxane (1.5 mL) and EtOH (1.5 mL) and the mixture refluxed for 3 h. Volatiles were removed in vacuo and to the suspension was added brine (2 mL), followed by 6 M HCl (2 mL) dropwise at 0° C. The resulting precipitate was filtered and washed with H₂O (2 mL), providing 5-((4-(4-(trifluoromethyl)phenyl)oxazol-2-yl)amino)pyrimidine-2-carboxylic acid as a yellow solid (0.150 g, 97.0% yield). ¹H NMR (400 MHz, DMSO-d₆) δ 11.26 (s, 1H), 9.27 (s, 2H), 8.50 (s, 2H), 8.04 (d, J=8.1 Hz, 2H), 7.81 (d, J=8.2 Hz, 2H). ¹³C NMR (101 MHz, DMSO-d₆) δ 164.87, 155.92, 139.61, 138.92, 138.45, 137.90, 135.03, 125.93, 125.67, 125.53, 122.50, 64.11, 52.06, 52.04. LCMS R_{f} (min)=3.811, MS m/z=348.9 [M−H]⁻.

A solution of 5-((4-(4-(trifluoromethyl)phenyl)oxazol-2-yl)amino)pyrimidine-2-carboxylic acid (0.100 g, 0.286 mmol), anhydrous HOBt (1.2 equiv.) and EDCI·HCl (1.3 equiv.) in dry DMF (2 mL) was stirred at rt for 2 h. After this time, O-benzylhydroxylamine hydrochloride (5 equiv.) and Et₃N (5 equiv.) were added and the mixture stirred at rt for a further 16 h. DMF was removed in vacuo, washing with toluene (2 mL×3) to aid this process. H₂O (2 mL) was then added and the mixture left to stir at rt for 10 min. The resulting precipitate was filtered and washed with H₂O (1 mL) and hexanes (2 mL), providing N-(benzyloxy)-5-((4-(4-(trifluoromethyl)phenyl)oxazol-2-yl)amino)pyrimidine-2-carboxamide as a brown solid (0.11 g, 84.6% yield). ¹H NMR (400 MHz, DMSO-d₆) δ 12.05 (s, 1H), 11.19 (s, 1H), 9.25 (s, 2H), 8.50 (s, 1H), 8.02 (d, J=8.1 Hz, 2H), 7.83 (d, J=8.3 Hz, 2H), 7.57-7.30 (m, 5H), 4.95-4.86 (m, 2H). ¹³C NMR (101 MHz, DMSO-d₆) δ 159.82, 155.60, 150.06, 144.24, 137.88, 135.84, 135.64, 134.87, 131.15, 128.73, 128.27, 128.24, 128.08, 127.76, 125.71, 125.48, 122.90, 77.02, 45.64, 8.61. LCMS R_{f} (min)=3.879, MS m/z=455.8 [M+H]⁺.

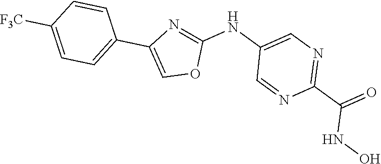 [SMILES: O=C(NO)c1ncc(Nc2nc(-c3ccc(C(F)(F)F)cc3)co2)cn1]

BBr₃ 1.0 M in heptane (3 equiv.) was added dropwise to a solution of N-(benzyloxy)-5-((4-(4-(trifluoromethyl)phenyl)oxazol-2-yl)amino)pyrimidine-2-carboxamide (0.110 g, 0.242 mmol) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The crude material was purified using preparative HPLC in a 95% A: 5% B to 100% B solvent system. The TFA and ACN were removed through rotary evaporation and H₂O through freeze-drying, providing the title compound as a white solid (0.035 g, 39.7% yield). ¹H NMR (400 MHz, DMSO-d₆) δ 11.43 (s, 1H), 11.13 (s, 1H), 9.23 (s, 2H), 9.15 (s, 1H), 8.50 (s, 1H), 8.02 (d, J=8.0 Hz, 2H), 7.83 (d, J=8.2 Hz, 2H). ¹³C NMR (101 MHz, DMSO-d₆) δ 160.44, 156.18, 151.31, 145.71, 144.76, 138.39, 135.81, 135.41, 131.59, 128.58, 128.26, 126.26, 126.22, 125.99, 123.39. LCMS R_{f} (min)=5.571. HRMS (ESI) calcd for C₁₅H₁₁F₃N₅O₃⁺ [M+H]⁺ 366.0809, found 366.0803.

### 13. N-Hydroxy-6-((4-(4-(trifluoromethyl)phenyl)oxazol-2-yl)amino)pyridazine-3-carboxamide (Scheme 11)

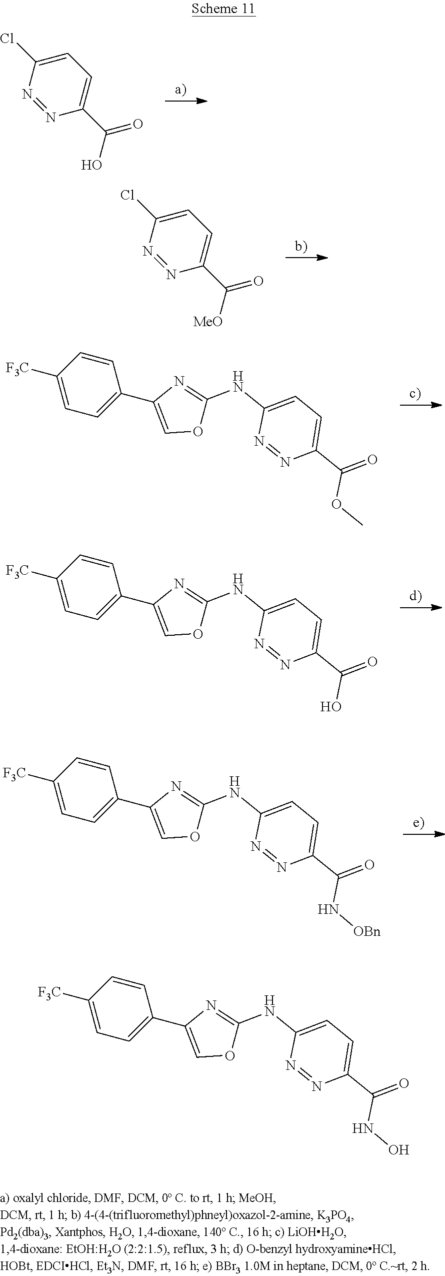

Oxalyl chloride (1 equiv.) was added dropwise to a solution of 6-chloro-3-pyridazinecarboxylic acid (1.000 g, 6.308 mmol) in dry DCM (30 mL) and dry DMF (1 drop) at 0° C. and the mixture stirred at this temperature for 1 h. The solvent was removed in vacuo and the residue redissolved in dry DCM. MeOH (1 equiv.) were added and the mixture stirred at rt for a further 1 h. The mixture was quenched with H₂O (20 mL) and extracted with DCM (2×20 mL). The combined organic layers were then dried over MgSO₄ and concentrated to give methyl 6-chloropyridazine-3-carboxylate as a white solid. (0.960 g, 88.2% yield). ¹H NMR (400 MHz, CDCl₃) δ 8.17 (d, J=8.8 Hz, 1H), 7.68 (d, J=8.8 Hz, 1H), 4.09 (s, 3H).

A re-sealable Schlenk tube was charged with Pd₂(dba)₃ (0.02 equiv.), Xantphos (0.06 equiv.), 4-(4-(trifluoromethyl)phenyl)oxazol-2-amine (Intermediate E) (1.2 equiv.), K₃PO₄ (fine powder, 1.4 equiv.), methyl 6-chloropyridazine-3-carboxylate (0.250 g, 1.152 mmol) and 1,4-dioxane (4 mL). After the mixture was degassed and carefully subjected to three cycles of evacuation and backfilling with N₂, H₂O (1.0 mmol) was added dropwise. This was then sealed and immersed in a 140° C. oil bath. After 15 h, the mixture was cooled, diluted with EtOAc (20 mL) and washed with H₂O (15 mL). The aq. layers were then back extracted with EtOAc (2×15 mL). The crude product was concentrated under reduced pressure and chromatographed on a SiO₂ column (EtOAc:toluene=1:1) to give methyl 6-((4-(4-(trifluoromethyl)phenyl)oxazol-2-yl)amino)pyridazine-3-carboxylate as a white solid (0.27 g, 51.2% yield). ¹H NMR (400 MHz, DMSO-d₆) δ 8.63 (d, J=8.8 Hz, 1H), 8.53 (s, 1H), 8.25 (d, J=9.1 Hz, 1H), 8.03 (d, J=7.9 Hz, 2H), 7.81 (d, J=8.3 Hz, 3H), 3.94 (s, 3H). ¹³C NMR (101 MHz, DMSO-d₆) δ 137.86, 134.86, 131.60, 130.00, 128.14, 127.82, 125.69, 125.58, 122.90, 52.57, 39.52. LCMS R_{f} (min)=3.496, MS m/z=364.8 [M+H]⁺.

LiOH·H₂O (3 equiv.) in H₂O (2 mL) was added to a solution of methyl 6-((4-(4-(trifluoromethyl)phenyl)oxazol-2-yl)amino)pyridazine-3-carboxylate (0.250 g, 0.686 mmol) in 1,4-dioxane (2 mL) and EtOH (2 mL) and the mixture refluxed for 3 h. Volatiles were removed in vacuo and to the suspension was added brine (2 mL), followed by 6 M HCl (2 mL) dropwise at 0° C. The resulting precipitate was filtered and washed with H₂O (2 mL), providing 6-((4-(4-(trifluoromethyl)phenyl)oxazol-2-yl)amino)pyridazine-3-carboxylic acid as a yellow solid (0.240 g, 99.8% yield). ¹H NMR (400 MHz, DMSO-d₆) δ 8.60 (s, 1H), 8.52 (s, 1H), 8.21 (s, 1H), 8.03 (d, J=8.1 Hz, 2H), 7.81 (d, J=8.3 Hz, 2H). ¹³C NMR (101 MHz, DMSO-d₆) δ 138.33, 135.38, 131.97, 130.56, 130.40, 128.77, 128.60, 128.28, 127.97, 126.15, 126.12, 126.05, 123.36. LCMS R_{f} (min)=3.742, MS m/z=350.8 [M+H]⁺.

A solution of 6-((4-(4-(trifluoromethyl)phenyl)oxazol-2-yl)amino)pyridazine-3-carboxylic acid (0.220 g, 0.628 mmol), anhydrous HOBt (1.2 equiv.) and EDCI·HCl (1.3 equiv.) in dry DMF (3 mL) was stirred at rt for 2 h. After this time, O-benzylhydroxylamine hydrochloride (5 equiv.) and Et₃N (5 equiv.) were added and the mixture stirred at rt for a further 16 h. DMF was removed in vacuo, washing with toluene (2 mL×3) to aid this process. H₂O (2 mL) was then added and the mixture left to stir at rt for 10 min. The resulting precipitate was filtered and washed with H₂O (1 mL) and hexanes (2 mL), providing N-(benzyloxy)-6-((4-(4-(trifluoromethyl)phenyl)oxazol-2-yl)amino)pyridazine-3-carboxamide as a brown solid (0.20 g, 69.9% yield)). ¹H NMR (400 MHz, DMSO-d₆) δ 12.36 (s, 1H), 8.65 (d, J=8.8 Hz, 1H), 8.54 (s, 1H), 8.19 (d, J=9.4 Hz, 1H), 8.04 (d, J=8.1 Hz, 2H), 7.82 (d, J=8.3 Hz, 2H), 7.54-7.46 (m, 2H), 7.45-7.33 (m, 3H), 4.98 (s, 2H). ¹³C NMR (101 MHz, DMSO-d₆) δ 160.71, 144.42, 136.27, 131.78, 129.27, 128.75, 128.29, 128.03, 126.58, 125.99, 125.40, 123.76, 123.29, 77.61. LCMS R_{f} (min)=3.852, MS m/z=455.8 [M+H]⁺.

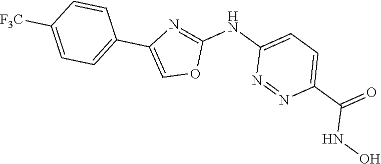 [SMILES: O=C(NO)c1ccc(Nc2nc(-c3ccc(C(F)(F)F)cc3)co2)nn1]

BBr₃ 1.0 M in heptane (3 equiv.) was added dropwise to a solution of N-(benzyloxy)-6-((4-(4-(trifluoromethyl)phenyl)oxazol-2-yl)amino)pyridazine-3-carboxamide (0.180 g, 0.395 mmol) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The crude material was purified using preparative HPLC in a 95% A: 5% B to 100% B solvent system. The TFA and ACN were removed through rotary evaporation and H₂O through freeze-drying, providing the title compound as a white solid (0.050 g, 34.6% yield). ¹H NMR (400 MHz, DMSO-d₆) δ 11.43 (s, 1H), 11.13 (s, 1H), 9.23 (s, 2H), 9.15 (s, 1H), 8.50 (s, 1H), 8.02 (d, J=8.0 Hz, 2H), 7.83 (d, J=8.2 Hz, 2H). LCMS R_{f} (min)=3.386. HRMS (ESI) calcd for C₁₅H₁₁F₃N₅O₃⁺ [M+H]⁺ 366.0809, found 366.0826.

### 14. N-Hydroxy-5-((4-[4-(trifluoromethyl)phenyl]-1,3-oxazol-2-yl)amino)picolinamide (Scheme 12)

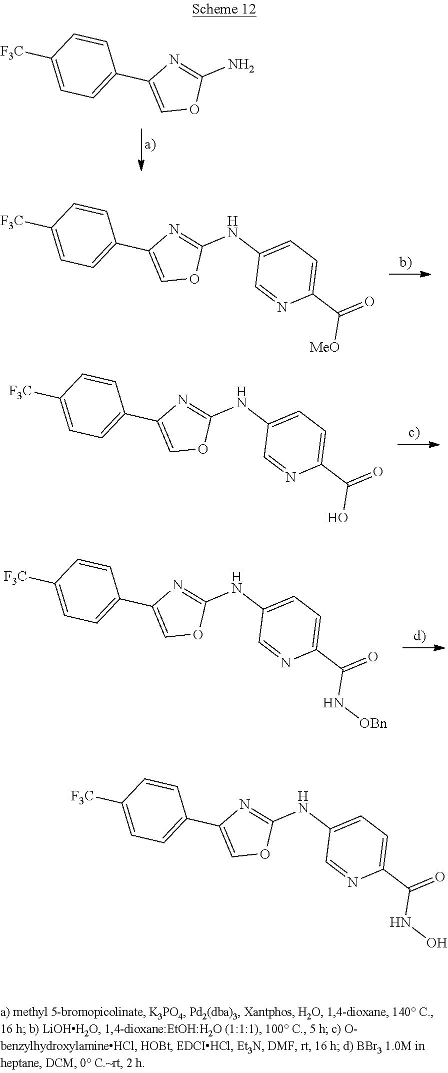 [SMILES: Nc1nc(-c2ccc(C(F)(F)F)cc2)co1 ; COC(=O)c1ccc(Nc2nc(-c3ccc(C(F)(F)F)cc3)co2)cn1 ; O=C(O)c1ccc(Nc2nc(-c3ccc(C(F)(F)F)cc3)co2)cn1 ; O=C(NOCc1ccccc1)c1ccc(Nc2nc(-c3ccc(C(F)(F)F)cc3)co2)cn1 ; O=C(NO)c1ccc(Nc2nc(-c3ccc(C(F)(F)F)cc3)co2)cn1]

A re-sealable Schlenk tube was charged with Pd₂(dba)₃ (0.02 equiv.), Xantphos (0.06 equiv.), 4-(4-(trifluoromethyl)phenyl)oxazol-2-amine (Intermediate E) (1.2 equiv.), K₃PO₄ (fine powder, 1.4 equiv.), methyl 5-bromopicolinate (0.250 g, 1.152 mmol) and 1,4-dioxane (4 mL). After the mixture was degassed and carefully subjected to three cycles of evacuation and backfilling with N₂, H₂O (1.0 mmol) was added dropwise. This was then sealed and immersed in a 140° C. oil bath. After 15 h, the mixture was cooled, diluted with EtOAc (20 mL) and washed with H₂O (15 mL). The aq. layers were then back extracted with EtOAc (2×15 mL). The crude product was concentrated under reduced pressure and chromatographed on a SiO₂ column (EtOAc:toluene=1:1) to give methyl 5-((4-(4-(trifluoromethyl)phenyl)oxazol-2-yl)amino)picolinate as a white solid (0.21 g, 49.8% yield). ¹H NMR (400 MHz, DMSO-d₆) δ 11.04 (s, 1H), 8.90 (d, J=2.5 Hz, 1H), 8.44 (s, 1H), 8.36 (dd, J=8.7, 2.6 Hz, 1H), 8.08 (d, J=8.6 Hz, 1H), 8.00 (d, J=8.1 Hz, 2H), 7.78 (d, J=8.4 Hz, 2H), 3.85 (s, 3H). ¹³C NMR (101 MHz, DMSO-d₆) δ 164.22, 155.55, 149.46, 144.33, 137.96, 135.69, 135.68, 134.91, 131.18, 125.74, 125.57, 125.55. LCMS R_{f} (min)=3.907, MS m/z=363.8 [M+H]⁺.

LiOH·H₂O (3 equiv.) in H₂O (2 mL) was added to a solution of methyl 5-((4-(4-(trifluoromethyl)phenyl)oxazol-2-yl)amino)picolinate (0.180 g, 0.495 mmol) in 1,4-dioxane (2 mL) and EtOH (2 mL) and the mixture refluxed for 3 h. Volatiles were removed in vacuo and to the suspension was added brine (2 mL), followed by 6 M HCl (2 mL) dropwise at 0° C. The resulting precipitate was filtered and washed with H₂O (2 mL), providing 5-((4-(4-(trifluoromethyl)phenyl)oxazol-2-yl)amino)picolinic acid as a yellow solid (0.240 g, 99.8% yield). ¹H NMR (400 MHz, DMSO-d₆) δ 11.01 (s, 1H), 8.90 (d, J=2.4 Hz, 1H), 8.46 (s, 1H), 8.34 (dd, J=8.6, 2.6 Hz, 1H), 8.07 (d, J=8.6 Hz, 1H), 8.02 (d, J=8.0 Hz, 2H), 7.81 (d, J=8.2 Hz, 2H).

¹³C NMR (101 MHz, DMSO-d₆) δ 166.27, 156.47, 146.55, 140.97, 139.20, 138.93, 138.75, 138.55, 138.36, 135.52, 131.28, 130.57, 128.94, 128.51, 128.19, 127.61, 127.05, 126.17, 125.99, 123.77, 123.39, 123.16. LCMS R_{f} (min)=3.578, MS m/z=349.8 [M+H]⁺.

A solution of 5-((4-(4-(trifluoromethyl)phenyl)oxazol-2-yl)amino)picolinic acid (0.110 g, 0.315 mmol), anhydrous HOBt (1.2 equiv.) and EDCI·HCl (1.3 equiv.) in dry DMF (3 mL) was stirred at rt for 2 h. After this time, O-benzylhydroxylamine hydrochloride (5 equiv.) and Et₃N (5 equiv.) were added and the mixture stirred at rt for a further 16 h. DMF was removed in vacuo, washing with toluene (2 mL×3) to aid this process. H₂O (2 mL) was then added and the mixture left to stir at rt for 10 min. The resulting precipitate was filtered and washed with H₂O (1 mL) and hexanes (2 mL), providing N-(benzyloxy)-5-((4-(4-(trifluoromethyl)phenyl)oxazol-2-yl)amino)picolinamide as a brown solid (0.14 g, 97.8% yield). ¹H NMR (400 MHz, DMSO-d₆) δ 11.86 (s, 1H), 10.97 (s, 1H), 8.91 (d, J=2.4 Hz, 1H), 8.45 (s, 1H), 8.32 (dd, J=8.7, 2.5 Hz, 1H), 8.01 (dd, J=8.4, 3.3 Hz, 2H), 7.82 (d, J=8.5 Hz, 2H), 7.51-7.24 (m, 5H), 4.94 (d, J=3.0 Hz, 2H). ¹³C NMR (101 MHz, DMSO-d₆) δ 162.01, 156.54, 142.58, 138.98, 138.35, 137.52, 136.46, 129.20, 128.75, 126.20, 125.97, 123.85, 123.39, 77.56, 77.51. LCMS R_{f} (min)=4.043, MS m/z=454.8 [M+H]⁺.

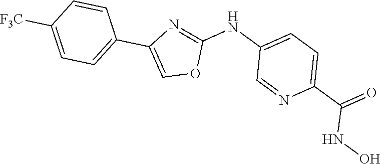 [SMILES: O=C(NO)c1ccc(Nc2nc(-c3ccc(C(F)(F)F)cc3)co2)cn1]

BBr₃ 1.0 M in heptane (3 equiv.) was added dropwise to a solution of N-(benzyloxy)-5-((4-(4-(trifluoromethyl)phenyl)oxazol-2-yl)amino)picolinamide (0.120 g, 0.264 mmol) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The crude material was purified using preparative HPLC in a 95% A: 5% B to 100% B solvent system. TFA and ACN were removed through rotary evaporation and H₂O through freeze-drying, providing the title compound as a white solid (0.029 g, 30.1% yield). ¹H NMR (400 MHz, DMSO-d₆) δ 11.23 (s, 1H), 10.91 (s, 1H), 8.89 (d, J=2.2 Hz, 1H), 8.45 (s, 1H), 8.29 (dd, J=8.6, 2.6 Hz, 1H), 8.02-7.97 (m, 3H), 7.82 (d, J=8.2 Hz, 2H), 5.76 (s, 1H). ¹³C NMR (101 MHz, DMSO-d₆) δ 161.41, 156.15, 142.74, 140.13, 138.08, 137.86, 137.02, 135.10, 130.66, 130.09, 129.27, 128.02, 127.70, 125.73, 125.69, 125.47, 123.37, 122.92, 122.50, 39.52. LCMS R_{f} (min)=5.908. HRMS (ESI) calcd for C₁₆H₁₂F₃N₄O₃⁺ [M+H]⁺ 365.0856, found 365.0861.

### 15. N-Hydroxy-N-methyl-5-((5-[4-(trifluoromethyl)phenyl]-1,3-oxazol-2-yl)amino)pyridine-2-sulfonamide (Scheme 13)

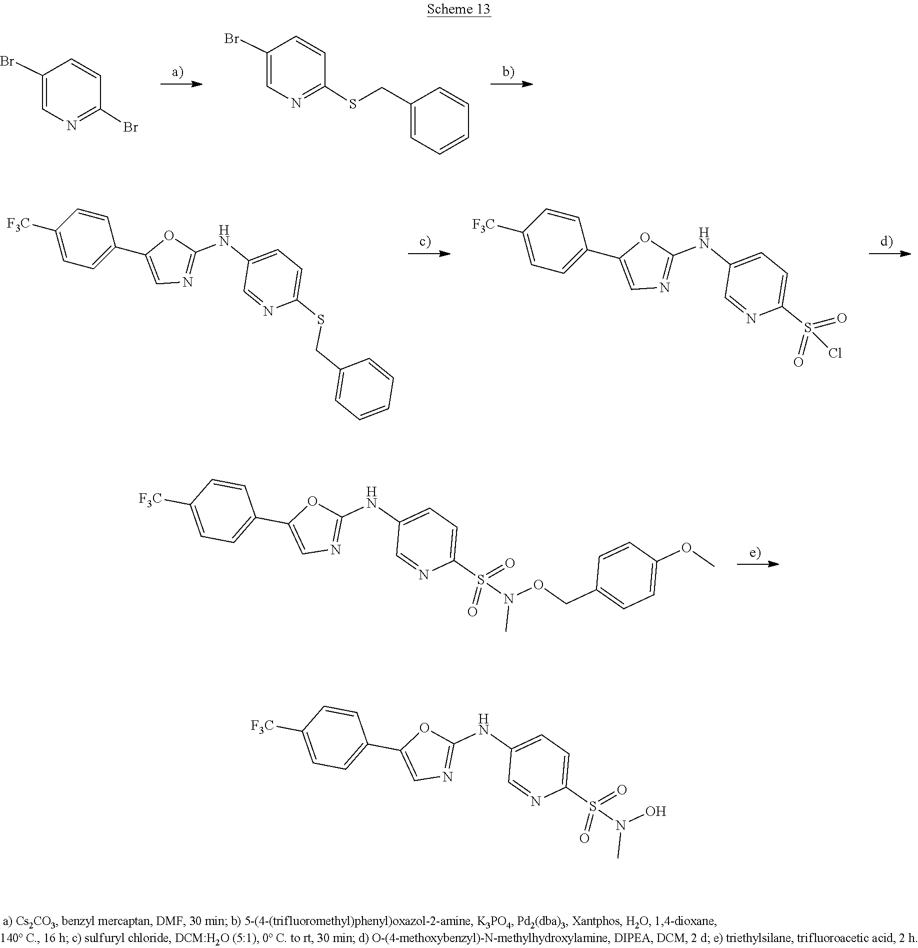 [SMILES: Brc1ccc(Br)nc1 ; Brc1ccc(SCc2ccccc2)nc1 ; FC(F)(F)c1ccc(-c2cnc(Nc3ccc(SCc4ccccc4)nc3)o2)cc1 ; O=S(=O)(Cl)c1ccc(Nc2ncc(-c3ccc(C(F)(F)F)cc3)o2)cn1 ; COc1ccc(CON(C)S(=O)(=O)c2ccc(Nc3ncc(-c4ccc(C(F)(F)F)cc4)o3)cn2)cc1 ; CN(O)S(=O)(=O)c1ccc(Nc2ncc(-c3ccc(C(F)(F)F)cc3)o2)cn1]

A solution of Cs₂CO₃ (29.227 g, 89.704 mmol) and benzyl mercaptan (10.511 mL, 89.704 mmol) in DMF (70 mL) stirred for 15 min. 2,5-dibromopyridine (5.000 g, 25.107 mmol) in DMF (30 mL) was added and the resulting solution stirred for 30 min at rt. The reaction mixture was diluted with water and extracted with EtOAc (3×20 mL). The combined organic layers were washed with brine and dried over anhydrous Na₂SO₄, filtered and concentrated under reduced pressure to yield crude 2-(benzylthio)-5-bromopyridine that was used without further purification. ¹H NMR (400 MHz, CDCl₃) δ 8.50 (dd, J=2.5, 0.7 Hz, 1H), 7.57 (dd, J=8.5, 2.4 Hz, 1H), 7.45-7.21 (m, 5H), 7.05 (dd, J=8.5, 0.7 Hz, 1H), 4.40 (s, 2H). LCMS R_{f} (min)=3.956. MS m/z 323.9 [M+H]⁺.

A re-sealable Schlenk tube was charged with Pd₂(dba)₃ (0.327 g, 0.357 mmol), Xantphos (0.620 g, 1.071 mmol), 5-(4-(trifluoromethyl)phenyl)oxazol-2-amine (Intermediate D) (0.977 g, 4.283 mmol), K₃PO₄ (fine powder, 1.061 g, 4.997 mmol), 2-(benzylthio)-5-bromopyridine (1.000 g, 3.569 mmol) and 1,4-dioxane (20 mL). After the mixture was degassed and carefully subjected to three cycles of evacuation and backfilling with N₂, H₂O (0.018 mL, 1.0 mmol) was added dropwise. This was then sealed and immersed in a 140° C. sand bath. After 15 h the volatiles were evaporated. The mixture was then filtered and washed with H₂O (10 mL), 10% potassium ethyl xanthate solution (10 mL) and ether (10 mL) to give N-(6-(benzylthio)pyridin-3-yl)-5-(4-(trifluoromethyl)phenyl)oxazol-2-amine as a grey solid (0.56 g, 43.1% yield). ¹H NMR (400 MHz, DMSO-d₆) δ 10.68 (s, 1H), 8.73 (d, J=2.3 Hz, 1H), 7.99 (dd, J=8.7, 2.6 Hz, 1H), 7.78 (s, 4H), 7.71 (s, 1H), 7.42-7.20 (m, 5H), 4.38 (s, 2H). ¹³C NMR (101 MHz, DMSO-d₆) δ 156.79, 149.31, 142.84, 138.50, 138.23, 133.20, 131.60, 128.81, 128.36, 128.27, 127.04, 126.94, 126.72, 126.04, 126.01, 125.57, 125.38, 125.06, 122.10, 33.97. LCMS R_{f} (min)=3.907, MS m/z=427.9 [M+H]⁺.

Sulfuryl chloride (0.185 g, 2.293 mmol) was added to a solution of N-(6-(benzylthio)pyridin-3-yl)-5-(4-(trifluoromethyl)phenyl)oxazol-2-amine (0.140 g, 0.328 mmol) in DCM (2 mL) and H₂O (0.5 mL) at 0° C. The reaction mixture slowly warmed to rt and stirred for 30 min under N₂. The grey suspension became yellow as SO₂Cl₂ was added gradually until it returned to grey suspension again. After the sulfonyl chloride was formed, the solvent was evaporated completely. The mixture was then redissolved in DCM (2 mL). DIPEA (0.285 mL, 0.742 mmol) and O-(4-methoxybenzyl)-N-methylhydroxylamine (0.066 mg, 0.393 mmol) was added into the suspension and the mixture was stirred at rt for 2 d. On completion, the mixture was extracted with EtOAc (3×20 mL). The organic layers were collected and washed with brine. The crude material (N-((4-methoxybenzyl)oxy)-N-methyl-5-((5-(4-(trifluoromethyl)phenyl)oxazol-2-yl)amino)pyridine-2-sulfonamide) was used in the next step without further purification (0.062 g, 35.4% yield). ¹H NMR (401 MHz, DMSO-d₆) δ 8.86 (d, J=2.5 Hz, 1H), 8.43 (dd, J=8.8, 2.6 Hz, 1H), 8.05 (d, J=8.7 Hz, 1H), 7.82 (s, 4H), 7.80 (s, 1H), 7.37-7.19 (m, 2H), 7.01-6.83 (m, 2H), 4.85 (s, 2H), 3.74 (s, 3H), 2.90 (s, 3H). LCMS R_{f} (min)=3.660, MS m/z=534.9 [M+H]⁺.

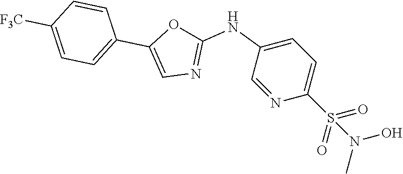 [SMILES: CN(O)S(=O)(=O)c1ccc(Nc2ncc(-c3ccc(C(F)(F)F)cc3)o2)cn1]

N-[(4-Methoxybenzyl)oxy]-N-methyl-5-((5-[4-(trifluoromethyl)phenyl]-1,3-oxazol-2-yl)amino)pyridine-2-sulfonamide was dissolved in 10% triethylsilane in trifluoroacetic acid (2 mL). The mixture was stirred at rt for 4 h. On completion, the mixture was filtered and washed with Et₂O (2 mL), providing a pale yellow solid. The crude material was purified using preparative HPLC. The TFA and ACN were removed through rotary evaporation and H₂O through the use of the freeze dryer, providing the title compound as a white solid (0.040 g, 83.2% yield). ¹H NMR (401 MHz, DMSO-d₆) 11.39 (s, 1H), 10.27 (s, 1H), 8.90 (d, J=2.5 Hz, 1H), 8.43 (dd, J=8.7, 2.5 Hz, 1H), 8.00 (d, J=8.7 Hz, 1H), 7.83 (s, 4H), 7.82 (s, 1H), 2.95 (s, 3H). LCMS R_{f} (min)=6.114. HRMS (ESI) calcd for C₁₆H₁₄F₃N₄O₄S⁺ [M+H]⁺ 415.0682, found 415.0687.

### 16. rac-N-(2,3-Dihydroxypropyl)-5-((5-[4-(trifluoromethyl)phenyl]-1,3-oxazol-2-yl)amino)pyridine-2-sulfonamide (Scheme 14)

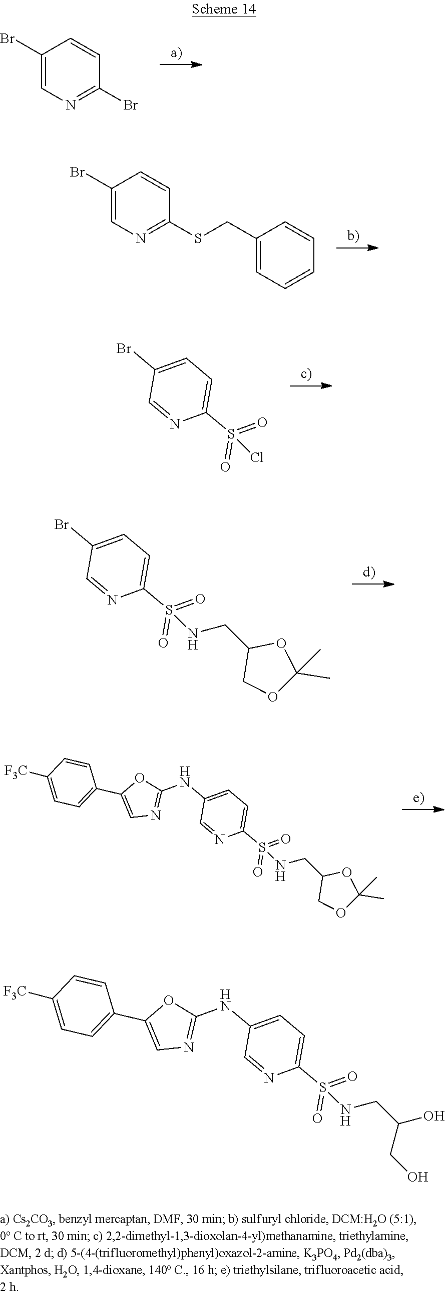 [SMILES: Brc1ccc(Br)nc1 ; Brc1ccc(SCc2ccccc2)nc1 ; O=S(=O)(Cl)c1ccc(Br)cn1 ; CC1(C)OCC(CNS(=O)(=O)c2ccc(Br)cn2)O1 ; CC1(C)OCC(CNS(=O)(=O)c2ccc(Nc3ncc(-c4ccc(C(F)(F)F)cc4)o3)cn2)O1 ; O=S(=O)(NCC(O)CO)c1ccc(Nc2ncc(-c3ccc(C(F)(F)F)cc3)o2)cn1]

A solution of Cs₂CO₃ (29.227 g, 89.704 mmol) and benzyl mercaptan (10.511 mL, 89.704 mmol) in DMF (70 mL) was stirred for 15 min. 2,5-Dibromopyridine (5.000 g, 25.107 mmol) in DMF (30 mL) was added and the resulting solution stirred for 30 min at rt. The reaction mixture was diluted with water and extracted with EtOAc (3×20 mL). The combined organic layers were washed with brine and dried over anhydrous Na₂SO₄, filtered and concentrated under reduced pressure to yield crude 2-(benzylthio)-5-bromopyridine that was used without further purification. ¹H NMR (400 MHz, CHCl₃) δ 8.50 (dd, J=2.5, 0.7 Hz, 1H), 7.57 (dd, J=8.5, 2.4 Hz, 1H), 7.45-7.21 (m, 5H), 7.05 (dd, J=8.5, 0.7 Hz, 1H), 4.40 (s, 2H). LCMS R_{f} (min)=3.956, MS m/z=323.9 [M+H]⁺.

Sulfuryl chloride (1.010 mL, 12.492 mmol) was added to a solution of 2-(benzylthio)-5-bromopyridine (0.500 g, 1.785 mmol) in DCM (10 mL) and H₂O (2 mL) at 0° C. The reaction mixture was slowly warmed to rt and stirred for 30 min. The mixture was then extracted with DCM (10 mL×3). The organic layers were combined and evaporated completely to give a grey solid. The solid was then redissolved with DCM (10 mL). A solution of rac-2,2-dimethyl-1,3-dioxolan-4-yl)methanamine and Et₃N in DCM (2 mL) was added dropwise at 0° C. The mixture was then stirred at rt for 2 h. On completion, the suspension was extracted with EtOAc (3×20 mL). The organic layers were collected and washed with brine. The crude product was further purified by silica column, eluting with 30% DCM in petroleum spirit to afford rac-5-bromo-N-((2,2-dimethyl-1,3-dioxolan-4-yl)methyl)pyridine-2-sulfonamide as a yellow solid (0.482 g, 69.98% yield). ¹H NMR (401 MHz, CHCl₃) δ 8.75 (dd, J=2.2, 0.6 Hz, 1H), 8.04 (dd, J=8.3, 2.3 Hz, 1H), 7.88 (dd, J=8.3, 0.7 Hz, 1H), 7.43-7.36 (m, 1H), 4.24 (qd, J=6.3, 4.0 Hz, 1H), 4.03 (dd, J=8.5, 6.4 Hz, 1H), 3.74 (dd, J=8.5, 5.9 Hz, 1H), 3.32 (dd, J=13.1, 4.0 Hz, 1H), 3.14 (dd, J=13.1, 6.4 Hz, 1H), 1.39 (s, 3H), 1.32 (s, 3H). ¹³C NMR (101 MHz, CDCl₃) δ 156.06, 151.32, 140.71, 130.78, 128.93, 124.43, 123.47, 109.82, 74.76, 74.36, 66.58, 59.19, 46.04, 26.88, 25.27. LCMS R_{f} (min)=3.770, MS m/z=292.8 [M+H]⁺.

A re-sealable Schlenk tube was charged with Pd₂(dba)₃ (0.033 g, 0.057 mmol), Xantphos (0.099 g, 0.171 mmol), 5-(4-(trifluoromethyl)phenyl)oxazol-2-amine (Intermediate D) (0.156 g, 0.683 mmol), K₃PO₄ (fine powder, 0.169 g, 0.797 mmol), rac-5-bromo-N-((2,2-dimethyl-1,3-dioxolan-4-yl)methyl)pyridine-2-sulfonamide (0.200 g, 0.569 mmol) and 1,4-dioxane (5 mL). After the mixture was degassed and carefully subjected to three cycles of evacuation and backfilling with N₂, H₂O (0.010 mL, 1.0 mmol) was added dropwise. This was then sealed and immersed in a 140° C. sand bath. After 15 h, the volatiles were evaporated. The mixture was then filtered and washed with H₂O (10 mL), 10% potassium ethyl xanthate solution (10 mL) and ether (10 mL) to give rac-N-((2,2-dimethyl-1,3-dioxolan-4-yl)methyl)-5-((5-(4-(trifluoromethyl)phenyl)oxazol-2-yl)amino)pyridine-2-sulfonamide as a yellow solid (0.164 g, 57.7% yield). ¹H NMR (401 MHz, DMSO-d₆) δ 11.26 (s, 1H), 8.85 (d, J=2.5 Hz, 1H), 8.35 (dd, J=8.7, 2.6 Hz, 1H), 7.94 (d, J=8.7 Hz, 1H), 7.89 (t, J=6.2 Hz, 1H), 7.81 (s, 4H), 7.79 (s, 1H), 4.12-4.00 (m, 1H), 3.93 (dd, J=8.4, 6.2 Hz, 1H), 3.66 (dd, J=8.4, 5.5 Hz, 1H), 3.14-2.89 (m, 2H), 1.27 (s, 3H), 1.21 (s, 3H). ¹³C NMR (101 MHz, DMSO-d₆) δ 154.23, 147.95, 141.66, 136.65, 136.50, 129.58, 125.94, 125.62, 125.30, 124.99, 124.28, 124.25, 123.73, 123.39, 121.79, 121.35, 120.95, 106.78, 72.51, 64.72, 43.79, 27.73, 24.97, 23.43.

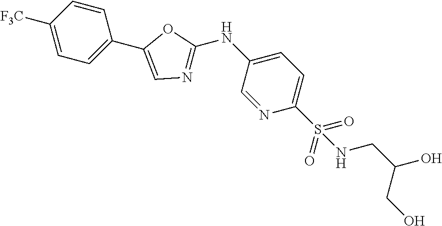 [SMILES: O=S(=O)(NCC(O)CO)c1ccc(Nc2ncc(-c3ccc(C(F)(F)F)cc3)o2)cn1]

rac-N-[(2,2-Dimethyl-1,3-dioxolan-4-yl)methyl]-5-((5-[4-(trifluoromethyl)phenyl]-1,3-oxazol-2-yl)amino)pyridine-2-sulfonamide was dissolved in 10% triethylsilane in trifluoroacetic acid (2 mL). The mixture was stirred at rt for 4 h. On completion, the mixture was filtered and washed with Et₂O (2 mL), providing the title compound as a pale yellow solid (0.102 g, 67.6% yield). ¹H NMR (401 MHz, DMSO-d₆) δ 11.27 (s, 1H), 8.85 (d, J=2.4 Hz, 1H), 8.35 (dd, J=8.7, 2.6 Hz, 1H), 7.93 (d, J=8.7 Hz, 1H), 7.81 (s, 3H), 7.79 (s, 1H), 7.50 (t, J=6.1 Hz, 1H), 3.62-3.38 (m, 10H), 3.32-3.20 (m, 3H), 3.10-2.95 (m, 1H), 2.85-2.74 (m, 1H). ¹³C NMR (101 MHz, DMSO-d₆) δ 156.53, 150.22, 143.91, 138.87, 138.68, 131.85, 127.86, 127.54, 126.54, 126.01, 125.68, 124.06, 123.63, 123.29, 70.99, 63.96, 46.84. LCMS R_{f} (min)=3.572, MS m/z=458.9 [M+H]⁺. HRMS (ESI) calcd for C₁₈H₁₈F₃N₄O₅S [M+H]⁺ 459.0945, found 459.0951.

### 17. rac-N-(2,3-Dihydroxypropyl)-5-((5-[4-(trifluoromethyl)phenyl]-1,3-oxazol-2-yl)amino) picolinamide (Scheme 15)

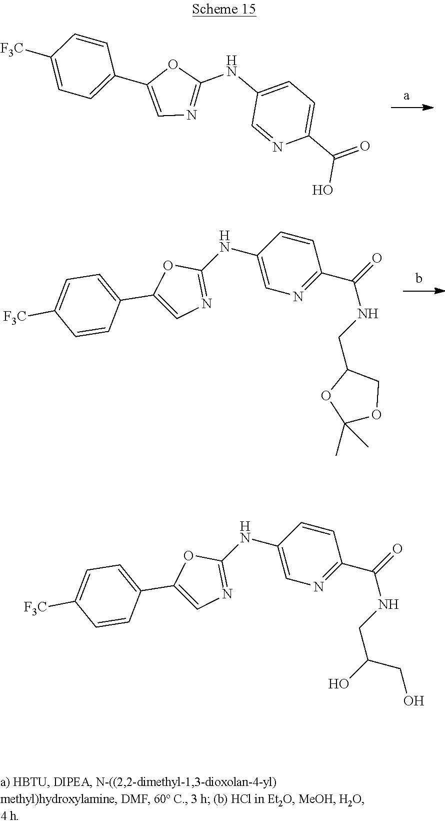 [SMILES: O=C(O)c1ccc(Nc2ncc(-c3ccc(C(F)(F)F)cc3)o2)cn1 ; CC1(C)OCC(CNC(=O)c2ccc(Nc3ncc(-c4ccc(C(F)(F)F)cc4)o3)cn2)O1 ; O=C(NCC(O)CO)c1ccc(Nc2ncc(-c3ccc(C(F)(F)F)cc3)o2)cn1]

HBTU (0.261 g, 0.687 mmol) and DIPEA (0.499 mL, 2.863 mmol) were added to a stirred solution of 5-((5-(4-(trifluoromethyl)phenyl)oxazol-2-yl)amino)picolinic acid (Intermediate B) (0.200 g, 0.573 mmol) in DMF (5 mL). The reaction mixture was stirred for 10 min at rt. An excess of rac-N-((2,2-dimethyl-1,3-dioxolan-4-yl)methyl)hydroxylamine (0.090 g, 0.687 mmol) was added and the resulting suspension was stirred for 3 h at 60° C. The reaction mixture was partition in H₂O and EtOA. The organic phase was separated, and washed aq. NaHCO₃, 10% w/v aq. citric acid and brine. The organic layer was dried with MgSO₄ and the solvent was removed under reduced pressure to afford the crude residue, which was subjected to column chromatography to give rac-N-((2,2-dimethyl-1,3-dioxolan-4-yl)methyl)-5-((5-(4-(trifluoromethyl)phenyl)oxazol-2-yl)amino)picolinamide (0.260 g, 98.3% yield). ¹H NMR (401 MHz, DMSO-d₆) δ 11.09 (s, 1H), 8.80 (d, J=2.2 Hz, 1H), 8.59 (t, J=6.2 Hz, 1H), 8.30 (dd, J=8.6, 2.6 Hz, 1H), 8.04 (d, J=8.6 Hz, 1H), 7.81 (s, 4H), 7.78 (s, 1H), 4.24 (p, J=5.8 Hz, 1H), 3.98 (dd, J=8.3, 6.3 Hz, 1H), 3.72 (dd, J=8.3, 5.6 Hz, 1H), 3.42 (t, J=6.1 Hz, 2H), 1.36 (s, 3H), 1.26 (s, 3H). ¹³C NMR (101 MHz, DMSO-d₆) δ 164.33, 156.70, 143.73, 143.04, 138.70, 137.61, 131.90, 127.74, 127.42, 126.53, 126.49, 126.02, 125.74, 123.99, 123.53, 123.15, 108.90, 74.64, 67.15, 42.06, 27.27, 25.73. LCMS R_{f} (min)=3.633, MS m/z=462.8 [M+H]⁺.

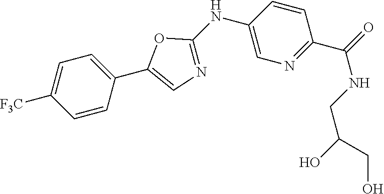 [SMILES: O=C(NCC(O)CO)c1ccc(Nc2ncc(-c3ccc(C(F)(F)F)cc3)o2)cn1]

HCl in Et₂O (0.054 mL, 0.216 mmol) was added dropwise to a solution of rac-N-((2,2-dimethyl-1,3-dioxolan-4-yl)methyl)-5-((5-[4-(trifluoromethyl)phenyl]-1,3-oxazol-2-yl)amino) picolinamide (0.050 g, 0.108 mmol) in MeOH (2 mL) and H₂O (0.5 mL). The mixture was stirred at rt for 4 h. On completion, the mixture was filtered and washed with Et₂O (2 mL), providing the title compound as a bright yellow solid (0.040 g, 87.6% yield). ¹H NMR (401 MHz, DMSO-d₆) δ 11.18 (s, 1H), 8.81 (s, 1H), 8.47 (s, 1H), 8.31 (d, J=8.6 Hz, 1H), 8.05 (d, J=8.6 Hz, 1H), 7.81 (s, 4H), 7.78 (s, 1H), 3.62 (dq, J=10.8, 5.5 Hz, 1H), 3.56-3.45 (m, 1H), 3.35 (ddd, J=26.5, 11.0, 5.6 Hz, 2H), 3.27-3.18 (m, 1H). ¹³C NMR (101 MHz, DMSO-d₆) δ 163.68, 156.26, 143.31, 142.65, 138.23, 137.12, 131.46, 128.28, 127.64, 127.32, 127.00, 126.68, 126.10, 126.06, 125.58, 125.27, 123.67, 123.10, 122.88, 122.62, 120.18, 70.26, 63.98, 42.40. LCMS R_{f} (min)=3.235, MS m/z=422.8 [M+H]⁺. HRMS (ESI) calcd for C₁₉H₁₈F₃N₄O₄[M+H]⁺ 423.1275, found 423.1285.

### 18. 6-((5-(4-(Trifluoromethyl)phenyl)oxazol-2-yl)amino)pyridin-3-ol (Scheme 16)

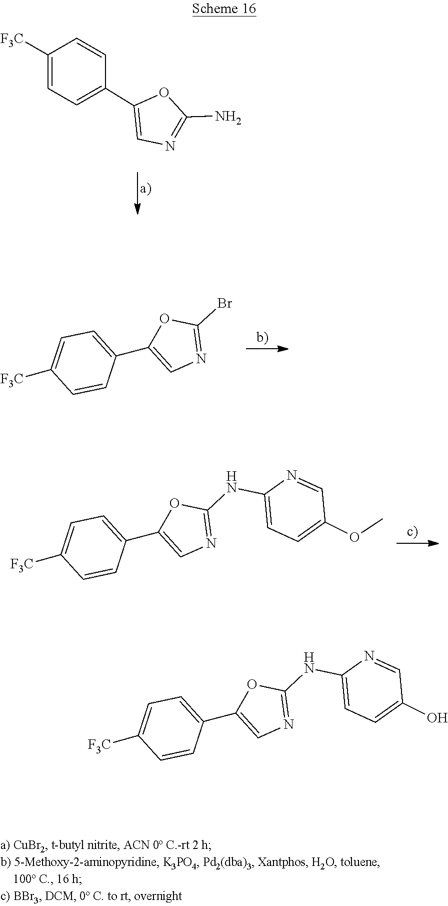 [SMILES: Nc1ncc(-c2ccc(C(F)(F)F)cc2)o1 ; FC(F)(F)c1ccc(-c2cnc(Br)o2)cc1 ; COc1ccc(Nc2ncc(-c3ccc(C(F)(F)F)cc3)o2)nc1 ; Oc1ccc(Nc2ncc(-c3ccc(C(F)(F)F)cc3)o2)nc1]

To a suspension of 5-(4-(trifluoromethyl)phenyl)oxazol-2-amine (Intermediate D) (0.51 g, 2.235 mmol) in ACN (4 mL) was added CuBr₂ (0.603 g, 2.839 mmol) at 0° C. The solution became dark green and tert-butyl nitrite (0.705 mL, 5.879 mmol) was added at 0° C. dropwise, whereupon the mixture was stirred at rt for 2 h. The reaction mixture was poured into water (5 mL) and DCM (5 mL) and the phases were separated. The aq. phase was extracted with DCM (3×5 mL), dried with Na₂SO₄ and evaporated to afford the crude product. Purification by column chromatography afforded 2-bromo-5-(4-(trifluoromethyl)phenyl)oxazole (0.153 g, 23.4% yield). ¹H NMR (401 MHz, DMSO-d₆) δ 7.93 (s, 1H), 7.88 (d, J=8.2 Hz, 2H), 7.81 (d, J=8.3 Hz, 2H). ¹³C NMR (101 MHz, DMSO-d₆) δ 153.25, 134.45, 130.11, 129.38, 129.06, 128.74, 128.42, 128.04, 126.98, 126.20, 126.16, 126.12, 126.09, 125.34, 124.48, 122.63. LCMS R_{f} (min)=3.633, MS m/z=291.7 [M+H]⁺.

A re-sealable Schlenk tube was charged with Pd₂(dba)₃ (0.047 g, 0.051 mmol), Xantphos (0.089 g, 0.154 mmol), 2-bromo-5-[4-(trifluoromethyl)phenyl]oxazole (0.150 g, 0.514 mmol), K₃PO₄ (fine powder, 0.153 g, 0.719 mmol), 5-methoxypyridin-2-amine (0.077 g, 0.616 mmol) and 1,4-dioxane (5 mL). After the mixture was degassed and carefully subjected to three cycles of evacuation and backfilling with N₂, H₂O (0.009 mL, 1.0 mmol) was added dropwise. The reaction was then sealed and immersed in a 140° C. sand bath. After 15 h, the volatiles were evaporated. The mixture was then filtered and washed with H₂O (10 mL), 10% potassium ethyl xanthate solution (10 mL) and ether (10 mL) to give a bright yellow solid (0.092 g). The crude product (N-(5-methoxypyridin-2-yl)-5-(4-(trifluoromethyl)phenyl)oxazol-2-amine) was used in the next step without further purification. ¹H NMR (401 MHz, DMSO) δ 10.84 (s, 1H), 8.03 (d, J=2.8 Hz, 1H), 8.00 (d, J=9.1 Hz, 1H), 7.83-7.76 (m, 4H), 7.71 (s, 1H), 7.48 (dd, J=9.1, 3.1 Hz, 1H), 3.82 (s, 3H). ¹³C NMR (101 MHz, DMSO) δ 157.28, 151.57, 146.12, 143.16, 134.83, 132.17, 127.43, 127.12, 126.54, 126.51, 126.47, 126.47, 126.43, 126.07, 125.72, 124.36, 123.31, 111.87, 56.32. LCMS R_{f} (min)=3.907, MS m/z=336.0 [M+H]⁺.

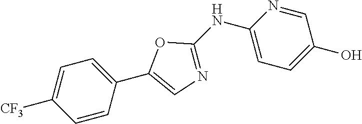 [SMILES: Oc1ccc(Nc2ncc(-c3ccc(C(F)(F)F)cc3)o2)nc1]

BBr₃ 1.0 M in heptane (0.079 mL, 0.823 mmol) was added dropwise to the crude N-(5-methoxypyridin-2-yl)-5-(4-(trifluoromethyl)phenyl)oxazol-2-amine (0.092 g) in dry DCM (1 mL) at 0° C. and the mixture stirred at rt for 2 h. Volatiles were removed in vacuo and the residue suspended in sat. NaHCO₃ (aq.) (3 mL) and stirred at rt for 20 min. The crude material was purified using preparative HPLC in 95% A: 5% B to 100% B solvent system. The TFA and ACN were removed through rotary evaporation and H₂O through freeze-drying, providing the title compound as a white solid (0.017 g, 19.3% yield). ¹H NMR (401 MHz, DMSO-d₆) δ 11.42 (s, 1H), 8.87 (d, J=2.2 Hz, 1H), 8.35 (dd, J=8.7, 2.7 Hz, 1H), 8.01 (d, J=8.6 Hz, 1H), 7.83 (s, 4H), 7.83 (s, 1H). LCMS R_{f} (min)=5.275. HRMS (ESI) calcd for C₁₅H₁₁F₃N₃O₂⁺ [M+H]⁺ 322.0798, found 322.0802.

### 19. N-(2-Hydroxyethyl)-5-((5-(4-(trifluoromethyl)phenyl)oxazol-2-yl)amino)picolinamide (Scheme 17)

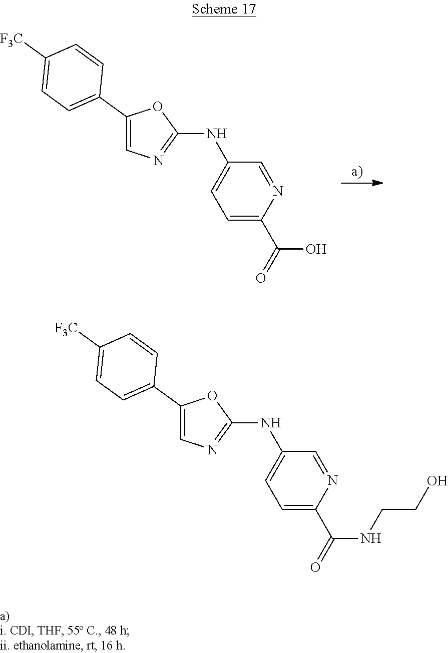 [SMILES: O=C(O)c1ccc(Nc2ncc(-c3ccc(C(F)(F)F)cc3)o2)cn1 ; O=C(NCCO)c1ccc(Nc2ncc(-c3ccc(C(F)(F)F)cc3)o2)cn1]

5-((5-(4-(Trifluoromethyl)phenyl)oxazol-2-yl)amino)picolinic acid (Intermediate B) (105 mg, 0.30 mmol) was suspended in dry THF, CDI (1.5 equiv.) was added and the resulting mixture was stirred at 55° C. for 24 h. More CDI (1 equiv.) was added and the stirring continued for an additional 24 h. Ethanolamine (10 equiv.) was added and the stirring continued overnight at rt. LCMS indicated that the reaction was complete. The volatiles were removed and the residue triturated with 1N HCl, the solid was collected via filtering and washing with H₂O (3×). The solid product was freeze-dried from DMSO/dioxane to provide the title product (containing ˜ molar 20% DMSO); 103 mg, 87.32%; ¹H NMR (401 MHz, DMSO-d₆) δ 11.10 (s, 1H), 8.80 (d, J=2.4 Hz, 1H), 8.50 (t, J=5.8 Hz, 1H), 8.29 (dd, J=8.6, 2.5 Hz, 1H), 8.03 (d, J=8.6 Hz, 1H), 7.81 (s, 4H), 7.78 (s, 1H), 4.80 (t, J=5.4 Hz, 1H), 3.52 (m, 2H), 3.37 (m, 2H). LCMS R_{f} (min)=4.081, MS n/z=393.0 [M+H]⁺. HRMS (ESI) calcd for C₁₈H₁₆F₃N₄O₃⁺ [M+H]⁺ 393.1169 found 393.1177

### 20. N-Hydroxy-N-methyl-5-((5-(4-(trifluoromethyl)phenyl)oxazol-2-yl)amino)picolinamide (Scheme 18)

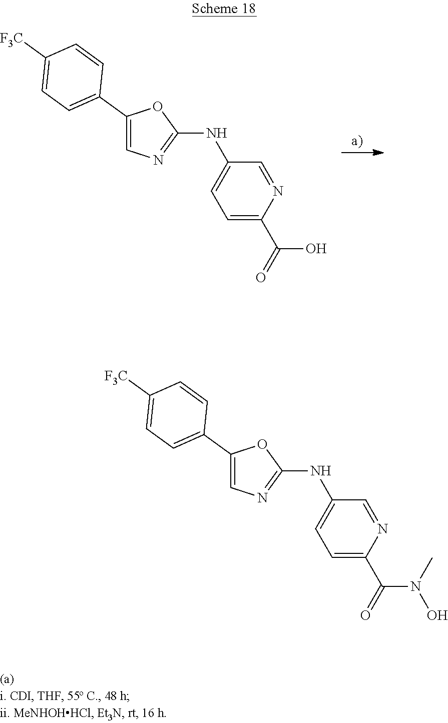 [SMILES: O=C(O)c1ccc(Nc2ncc(-c3ccc(C(F)(F)F)cc3)o2)cn1 ; CN(O)C(=O)c1ccc(Nc2ncc(-c3ccc(C(F)(F)F)cc3)o2)cn1]

5-((5-(4-(Trifluoromethyl)phenyl)oxazol-2-yl)amino)picolinic acid (Intermediate B) (92 mg, 0.263 mmol) was suspended in dry THF, CDI (1.5 equiv.) added and the resulting mixture was stirred at 55° C. for 24 h. More CDI (1 equiv.) was added and the stirring continued for an additional 24 h. MeNHOH·HCl and Et₃N (10 equiv. each) were added and the stirring continued overnight at rt. The volatiles were removed, the solid was collected via filtering and washing with H₂O (3×). The residual solid product was purified on basic ion exchange resin (Dowex@66) eluting with MeOH, then MeOH containing 0.1% Et₃N. The fractions containing products were pooled, evaporated to dryness and then purified on prep HPLC to provide the title compound (69 mg, 69.24% yield). ¹H NMR (401 MHz, DMSO-d₆) δ 11.06 (s, 1H), 8.80 (s, 1H), 8.24 (d, J=8.5 Hz, 1H), 7.81 (s, 4H), 7.77 (s, 1H), 7.72 (br, 1H), 5.85 (br, 1H), 3.33 (s, 3H). LCMS R_{f} (min)=4.130, MS m/z=378.9 [M+H]⁺. HRMS (ESI) calcd for C₁₇H₁₄F₃N₄O₃⁺ [M+H]⁺ 379.1013, found 379.1018.

### 21. N-Hydroxy-N,3-dimethyl-5-((5-(4-(trifluoromethyl)phenyl)oxazol-2-yl)amino)picolinamide (Scheme 19)

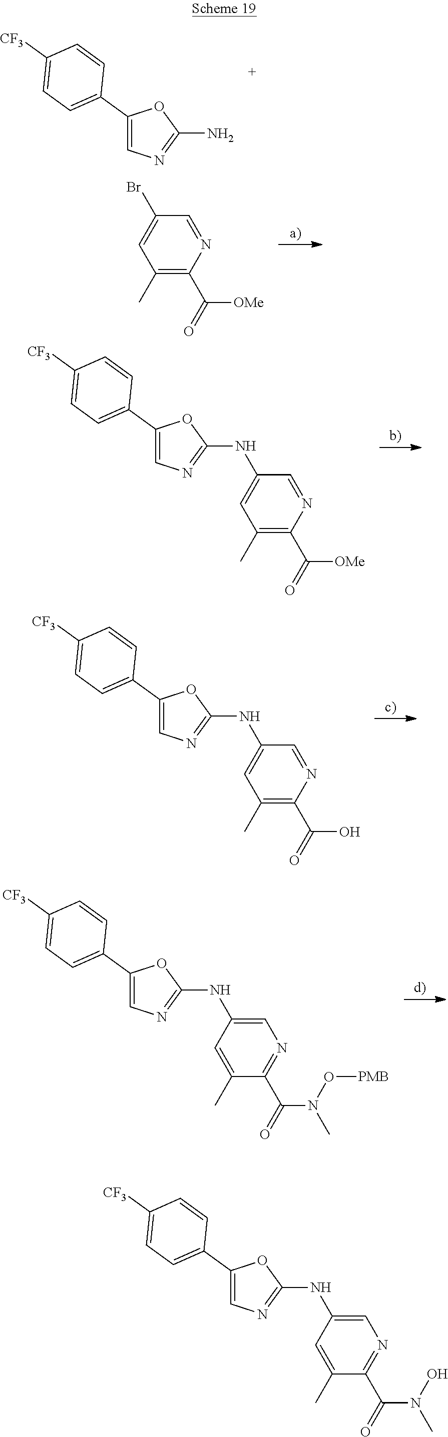

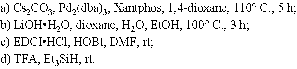

Intermediate D (5-(4-(trifluoromethyl)phenyl)oxazol-2-amine) (0.8 g, 3.506 mmol) was reacted with methyl 5-bromo-3-methylpicolinate (1.0 g, 4.382 mmol) as General Procedure 4 Method 1. Upon completion, the volatile solvents were removed in vacuo, and the residue treated with water and 1N HCl to adjust pH to 4-5. The solid precipitates were collected via filtering, washed with 5% potassium xanthate (2×), water (5×), diethyl ether (5×) to provide methyl 3-methyl-5-((5-(4-(trifluoromethyl)phenyl)oxazol-2-yl)amino)picolinate in 83.75% yield (1.108 g). ¹H NMR (401 MHz, DMSO-d₆) δ 11.07 (s, 1H), 8.64 (s, 1H), 8.07 (s, 1H), 7.78 (s, 4H), 7.76-7.74 (m, 1H), 3.81 (s, 3H), 2.50 (s, 3H). ¹³C NMR (101 MHz, DMSO-d₆) δ 166.42, 156.59, 143.74, 139.76, 138.24, 136.28, 136.19, 131.88, 127.74, 127.42, 126.53, 126.50, 126.01, 125.73, 125.53, 123.52, 123.31, 52.20, 20.40. LCMS R_{f} (min)=3.807. HRMS (ESI) calcd for C₁₈H₁₅F₃N₃O₃⁺ [M+H]⁺ 378.1060, found 378.1065.

Methyl 3-methyl-5-((5-(4-(trifluoromethyl)phenyl)oxazol-2-yl)amino)picolinate (0.297 g, 0.787 mmol) was hydrolysed as per step b, Scheme 12 to provide 3-methyl-5-((5-(4-(trifluoromethyl)phenyl)oxazol-2-yl)amino)picolinic in 93.71% yield (0.268 g). ¹H NMR (401 MHz, DMSO-d₆) δ 11.07 (s, 1H), 8.67 (s, 1H), 8.07 (s, 1H), 7.79 (s, 5H), 2.54 (s, 3H). ¹³C NMR (101 MHz, DMSO-d₆) δ 167.08, 156.62, 143.74, 140.05, 138.29, 136.25, 135.69, 131.88, 127.74, 127.43, 126.53, 126.50, 126.01, 125.91, 125.74, 123.52, 123.31, 20.52. HRMS (ESI) calcd for C₁₇H₁₃F₃N₃O₃⁺ [M+H]⁺ 364.0904, found 364.0912. LCMS R_{f} (min)=3.695, MS m/z=363.9 [M+H]⁺.

3-Methyl-5-((5-(4-(trifluoromethyl)phenyl)oxazol-2-yl)amino)picolinic acid (0.248 g, 0.682 mmol) was coupled with O-(4-methoxybenzyl)-N-methylhydroxylamine hydrochloride as per step c, Scheme 12 to provide N-((4-methoxybenzyl)oxy)-N,3-dimethyl-5-((5-(4-(trifluoromethyl)phenyl)oxazol-2-yl)amino)picolinamide in 60.88% yield (0.213 g). ¹H NMR (401 MHz, CDCl₃) δ 9.10 (brs, 1H), 8.55 (s, 1H), 7.98 (d, J=1.8 Hz, 1H), 7.60 (s, 4H), 7.29 (s, 1H), 6.89 (s, 2H), 6.76 (s, 2H), 4.73 (s, 2H), 3.72 (s, 3H), 3.38 (s, 3H), 2.32 (s, 3H). ¹³C NMR (101 MHz, CDCl₃) δ 160.05, 156.50, 144.45, 135.87, 131.08, 129.28, 128.95, 125.95, 125.92, 125.77, 125.35, 123.43, 122.99, 122.65, 113.93, 55.23, 18.05. LCMS R_{f} (min)=3.479. HRMS (ESI) calcd for C₂₆H₂₄F₃N₄O₄⁺ [M+H]⁺ 513.1744, found 513.1726.

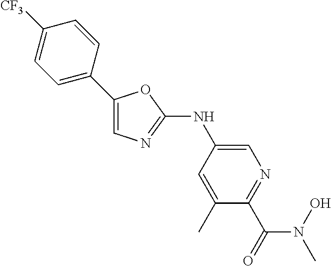 [SMILES: Cc1cc(Nc2ncc(-c3ccc(C(F)(F)F)cc3)o2)cnc1C(=O)N(C)O]

N-((4-Methoxybenzyl)oxy)-N,3-dimethyl-5-((5-(4-(trifluoromethyl)phenyl)oxazol-2-yl)amino)picolinamide (0.193 g, 0.376 mmol) was subjected to deprotection as per General procedure 11 in TFA and Et₃SiH to provide N-hydroxy-N,3-dimethyl-5-((5-(4-(trifluoromethyl)phenyl)oxazol-2-yl)amino)picolinamide in 79.18% yield (0.117 g). ¹H NMR (401 MHz, DMSO) δ 10.78 (s, 1H: 2 peaks due to isomers), 9.98 (brs, 1H), 8.60 (s, 2H), 7.98 (s, 1H: 2 peaks due to isomers), 7.78 (s, 4H), 7.73 (s, 1H), 3.28 (s, 2H: of major isomer), 3.05 (m, 1H: of minor isomer), 2.25 (s, 3H: 2 peaks due to isomers). ¹³C NMR (101 MHz, DMSO) δ 167.88, 157.10, 147.75, 143.41, 136.04, 135.72, 135.19, 131.98, 130.52, 128.71, 127.60, 127.28, 126.96, 126.50, 126.46, 126.02, 125.75, 125.22, 123.39, 123.32, 120.62, 36.07, 17.82. LCMS R_{f} (min)=3.234, MS m/z=392.9 [M+H]⁺. HRMS (ESI) calcd for C₁₈H₁₆F₃N₄O₃⁺ [M+H]⁺ 393.1169, found 393.1178.

### 22. N-Hydroxy-N-(2-hydroxyethyl)-5-((5-(4-(trifluoromethyl)phenyl)oxazol-2-yl)amino)picolinamide (Scheme 20)

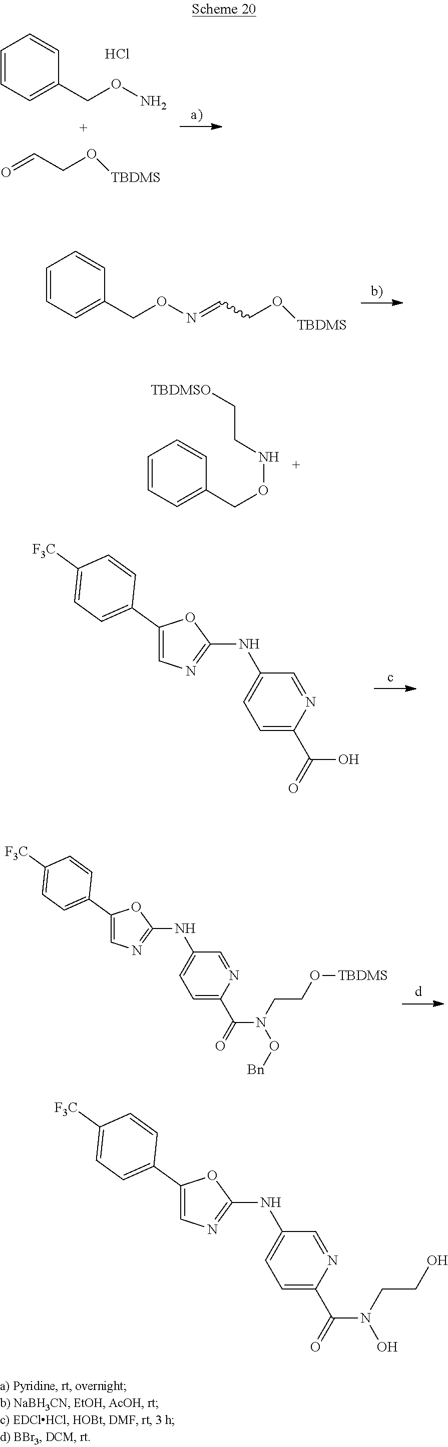

2-((tert-Butyldimethylsilyl)oxy)acetaldehyde (0.8 g, 4.59 mmol) was stirred with O-benzylhydroxylamine hydrochloride salt (1.1 g, 6.88 mmol) in pyridine (9 mL) overnight at rt. The reaction was deemed complete by LCMS. Volatile pyridine was removed on the rotavap and the residue was taken up in DCM and sat. NaHCO₃ solution. The phases were separated and the aq. phase was extracted with additional DCM (3×). The combined organic phases were dried over MgSO₄ and evaporated to dryness. The residue was purified on silica gel with neat DCM to provide the product 2-((tert-butyldimethylsilyl)oxy)acetaldehyde O-benzyl oxime as a ˜1:1 mixture of E and Z isomers (1.20 g, 93.5% yield). ¹H NMR (401 MHz, CDCl₃; chemical shifts of both are assigned) δ 7.48 (t, J=5.6 Hz, 1H), 7.38-7.28 (m, 10H), 6.83 (t, J=3.4 Hz, 1H), 5.09 (s, 2H), 5.07 (s, 2H), 4.48 (d, J=3.4 Hz, 2H), 4.25 (d, J=5.6 Hz, 2H), 0.91-0.90 (m, 9H), 0.88 (d, J=2.9 Hz, 9H), 0.07 (s, 6H), 0.06 (s, 6H). LCMS R_{f} (min)=3.95, MS m/z=280.0 [M+H]⁺.

2-((tert-Butyldimethylsilyl)oxy)acetaldehyde O-benzyl oxime (1.14 g, 4.08 mmol) was dissolved in a mixture of EtOH (20 mL) and acetic acid (4 mL); NaBH₃CN (5 equiv.) was added portion-wise and the resulting reaction mixture was stirred overnight at rt. The reaction was deemed complete by LCMS. The reaction mixture was cooled down to 0° C. and neutralized with 2N NaOH then diluted further with sat. NaHCO₃ solution. The product was extracted with DCM (3×), which was dried over MgSO₄ and evaporated to dryness. The residue was purified on silica gel with petroleum spirits/DCM to provide O-benzyl-N-(2-((tert-butyldimethylsilyl)oxy)ethyl)hydroxylamine (1.01 g, 87.96% yield). ¹H NMR (401 MHz, CDCl₃) δ 7.38-7.27 (m, 5H), 6.5-5.5 (brs, 1H), 4.72 (s, 2H), 3.74 (t, J=5.3 Hz, 2H), 3.02 (t, J=5.3 Hz, 2H), 0.88 (s, 9H), 0.05 (s, 6H). LCMS R_{f} (min)=4.426, MS m/z=282.1 [M+H]⁺.

5-((5-(4-(Trifluoromethyl)phenyl)oxazol-2-yl)amino)picolinic acid (Intermediate B) (200 mg, 0.572 mmol) was suspended in DMF (11 mL), EDCI·HCl (1.3 equiv.) and HOBt (1.4 equiv.) added and the resulting mixture was stirred at rt for 3 h. O-Benzyl-N-(2-((tert-butyldimethylsilyl)oxy)ethyl)hydroxylamine (2 equiv.) was added and the stirring continued overnight. The reaction mixture was diluted with sat. NaHCO₃ solution and EtOAc. After separation, the aq. phase was extracted with further EtOAc (3×). The combined organic phases were dried over MgSO₄ and evaporated to dryness. The residue was purified on silica gel using toluene and EtOAc mixtures as eluents to give the product N-(benzyloxy)-N-(2-((tert-butyldimethylsilyl)oxy)ethyl)-5-((5-(4-(trifluoromethyl)phenyl)oxazol-2-yl)amino)picolinamide (263 mg, 74.95% yield). ¹H NMR (401 MHz, CDCl₃) δ 8.69 (d, J=2.3 Hz, 1H), 8.14 (dd, J=8.6, 2.6 Hz, 1H), 7.80 (s, 1H), 7.60 (m, 4H), 7.28 (m, 5H), 5.02 (s, 2H), 3.99 (t, J=5.7 Hz, 2H), 3.85 (m, 2H), 0.82 (s, 9H), −0.00 (s, 6H). LCMS R_{f} (min)=4.001, MS m/z=613.0 [M+H]⁺.

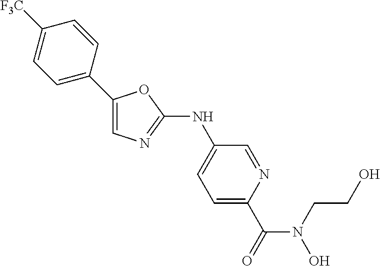 [SMILES: O=C(c1ccc(Nc2ncc(-c3ccc(C(F)(F)F)cc3)o2)cn1)N(O)CCO]

N-(Benzyloxy)-N-(2-((tert-butyldimethylsilyl)oxy)ethyl)-5-((5-(4-(trifluoromethyl)phenyl)oxazol-2-yl)amino)picolinamide (320 mg, 0.522 mmol) was suspended in DCM (2.6 mL) and of BBr₃ (2.5 equiv. of 0.5 M DCM solution) was added at 0° C. The reaction mixture was stirred at rt. After 1 h more BBr₃ (1.2 equiv.) added and the stirring continued. LCMS indicated that after 1 h there was still some benzyl-protected intermediate (˜15%). More BBr₃ (1.2 equiv., total=4.8 equiv.) added. After an additional 1 h of stirring the volatiles were removed in vacuo and the residue treated with MeOH containing 1% DIPEA until pH˜9. MeOH was removed and the residue stirred in H₂O/DMSO (1/1) for 0.5 h then run through a C₁₈ silica gel plug. The crude product was purified by preparative HPLC. The fractions containing the desired product were combined, diluted with dioxane and freeze-dried to provide the title compound (83 mg, 31.88% yield), with the product still containing a trace of dioxane. ¹H NMR (401 MHz, DMSO-d₆) δ 11.32 (s, 1H), 8.86 (d, J=2.5 Hz, 1H), 8.31 (dd, J=8.7, 2.6 Hz, 1H), 8.20 (d, J=8.7 Hz, 1H), 7.80 (s, 4H), 7.79 (s, 1H), 4.58-4.54 (m, 2H), 3.61-3.57 (m, 2H). LCMS R_{f} (min)=3.060, MS m/z=408.9[M+H]⁺. HRMS (ESI) calcd for C₁₈H₁₆F₃N₄O₄⁺ [M+H]⁺ 409.1118 found 409.1129.

### 23. 1-(Hydroxy)-4-((5-(4-(trifluoromethyl)phenyl)oxazol-2-yl)amino)pyridin-2(1H)-one (Scheme 21)

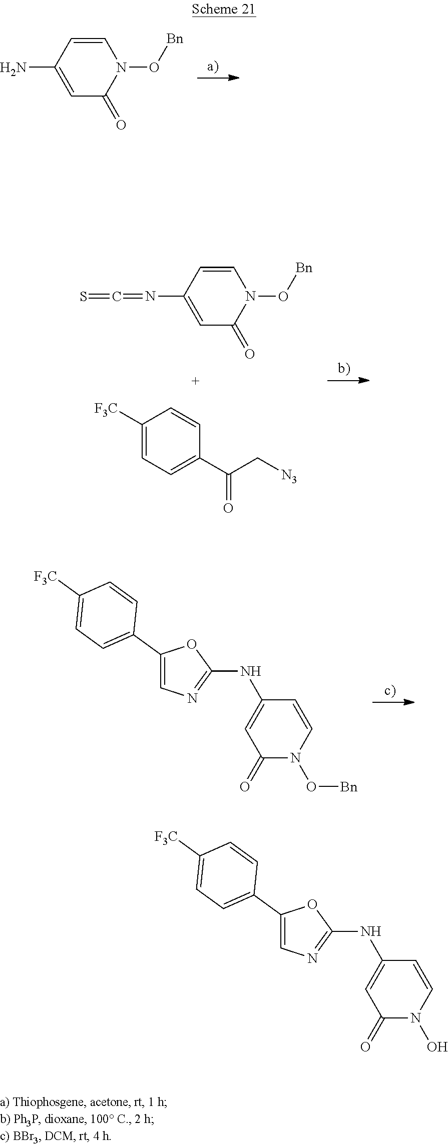 [SMILES: Nc1ccn(OCc2ccccc2)c(=O)c1 ; O=c1cc(N=C=S)ccn1OCc1ccccc1 ; [N-]=[N+]=NCC(=O)c1ccc(C(F)(F)F)cc1 ; O=c1cc(Nc2ncc(-c3ccc(C(F)(F)F)cc3)o2)ccn1OCc1ccccc1 ; O=c1cc(Nc2ncc(-c3ccc(C(F)(F)F)cc3)o2)ccn1O]

4-Amino-1-(benzyloxy)pyridin-2(1H)-one (240 mg, 1.11 mmol) was suspended in a mixture of acetone (11 mL) and sat. NaHCO₃ solution (11 mL) and cooled to 0° C. Thiophosgene (1.2 equiv.) was added dropwise and the resultant mixture was stirred at rt for 1 h. The reaction was deemed complete by TLC (neat EtOAc). The reaction mixture was poured into sat. NaHCO₃ solution and the product extracted with DCM (5×). The combined organic phases were dried over MgSO₄ and evaporated to dryness. The residue was dissolved in dioxane (5.5 mL), 2-azido-1-(4-(trifluoromethyl)phenyl)ethan-1-one (Intermediate A) and Ph₃P (1.25 equiv. each) were added. The resulting mixture was stirred at reflux for 2 h. All volatile solvents were removed and the residue triturated with DCM. The solids were filtered with DCM and washed with Et₂O to provide the product 1-(benzyloxy)-4-((5-(4-(trifluoromethyl)phenyl)oxazol-2-yl)amino)pyridin-2(1H)-one as a creamy solid (206 mg, 43.53% yield). ¹H NMR (401 MHz, DMSO-d₆) δ 10.90 (s, 1H), 7.81 (m, 4H), 7.78 (s, 1H), 7.66 (d, J=7.8 Hz, 1H), 7.51-7.44 (m, 2H), 7.44-7.38 (m, 3H), 6.99 (d, J=2.8 Hz, 1H), 6.21 (dd, J=7.8, 2.8 Hz, 1H), 5.17 (s, 2H). LCMS R_{f} (min)=3.406, MS m/z=428.0 [M+H]⁺.

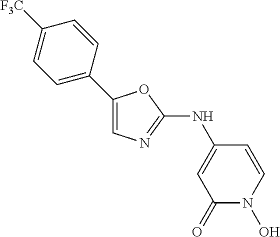 [SMILES: O=c1cc(Nc2ncc(-c3ccc(C(F)(F)F)cc3)o2)ccn1O]

1-(Benzyloxy)-4-((5-(4-(trifluoromethyl)phenyl)oxazol-2-yl)amino)pyridin-2(1H)-one (100 mg, 0.233 mmol) was suspended in DCM then cooled to 0° C. BBr₃ (0.089 mL; 0.935 mmol) was added dropwise. After 4 h of stirring at rt, LCMS indicated that all of the starting material had been consumed. The volatile solvents were removed and the residue quenched with sat. NaHCO₃ solution and MeOH for 1 h. The volatiles were removed and the residue purified by preparative HPLC to provide the title compound (32 mg, 40.5% yield). ¹H NMR (401 MHz, DMSO-d₆) δ 10.84 (s, 1H), 7.80 (s, 5H), 7.76 (s, 1H), 6.94 (s, 1H), 6.32 (d, J=5.6 Hz, 1H). ¹³C NMR (101 MHz, DMSO-d₆) δ 158.49, 155.85, 146.34, 143.30, 135.71, 131.39, 127.40, 127.09, 126.11, 126.07, 125.54, 125.21, 123.18, 122.84, 100.42, 96.73. LCMS R_{f} (min)=3.199. HRMS (ESI) calcd for C₁₅H₁₁F₃N₃O₃⁺ [M+H]⁺ 338.0747, found 338.0743

### 24. N′-Hydroxy-5-((1-(4-(trifluoromethyl)phenyl)-1H-1,2,4-triazol-3yl)amino)picolinimidamide (Scheme 22)

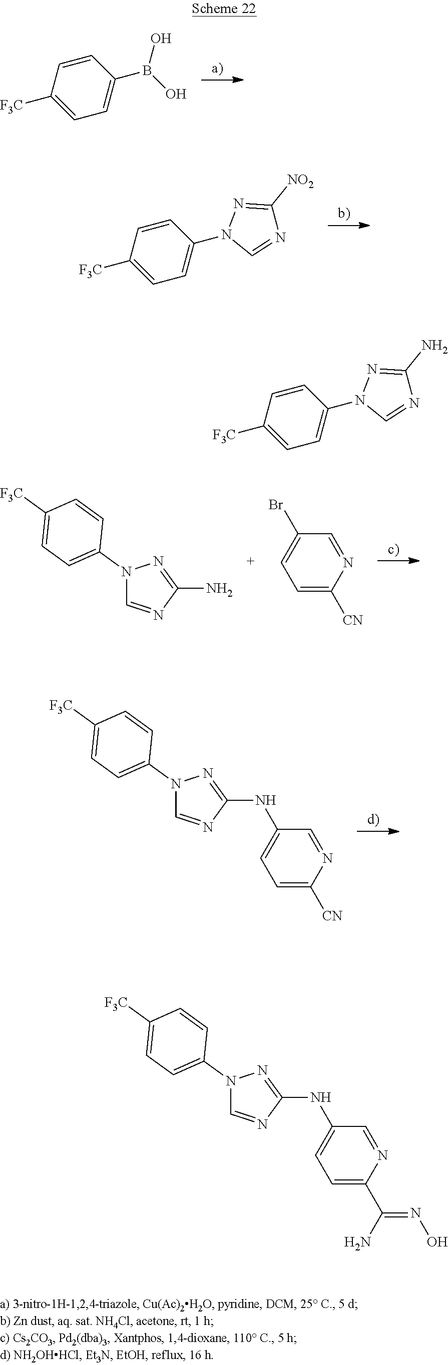

4-(Trifluoromethyl)phenylboronic acid (6.661 g, 35.05 mmol), Cu(OAc)₂·H₂O (5.251 g, 26.32 mmol) and pyridine (2.825 mL, 35.06 mmol) were added to a solution of 3-nitro-1H-1,2,4-triazole (2.0 g, 17.54 mmol) in DCM (100 mL). The resulting solution was stirred for 5 d at 25° C. The solvent was removed under reduced pressure to obtain the crude product. The crude product was purified over a SiO₂ column using a Reveleris X₂ flash chromatography system (EtOAc:petroleum spirit=2-2.5:10) to obtain 3-nitro-1-(4-(trifluoromethyl)phenyl)-1H-1,2,4-triazole as a white solid (2.55 g, 56.3% yield). ¹H NMR (401 MHz, CDCl₃) δ 8.71 (s, 1H), 7.93 (d, J=8.6 Hz, 2H), 7.88 (d, J=8.6 Hz, 2H).

Intermediate F synthesis—1-(4-(trifluoromethyl)phenyl)-1H-1,2,4-triazol-3-amine) Zinc dust (3.039 g) was added slowly to the stirring suspension of 3-nitro-1-(4-(trifluoromethyl)phenyl)-1H-1,2,4-triazole (2.4 g) in aq. sat. NH₄Cl (25 mL) and acetone (100 mL) at 0° C. After the complete addition, the ice bath was removed and the mixture was stirred for 1 h while warming up to rt. The mixture was filtered and the filtrate was concentrated under reduced pressure, then diluted with EtOAc (200 mL). The EtOAc layer was separated, washed with aq. sat. NaHCO₃ (30 mL) and brine (2×50 mL) and again separated, dried over MgSO₄, filtered and concentrated in vacuo to give 1-(4-(trifluoromethyl)phenyl)-1H-1,2,4-triazol-3-amine (2.0 g, 94.3% yield). The obtained compound was used without any purification. ¹H NMR (401 MHz, DMSO-d₆) δ 8.98 (s, 1H), 7.93 (d, J=8.6 Hz, 2H), 7.85 (d, J=8.7 Hz, 2H), 5.85 (s, 2H).

An oven-dried RBF was charged with Pd₂(dba)₃ (0.05 equiv.), Xantphos (0.1 equiv.), 1-(4-(trifluoromethyl)phenyl)-1H-1,2,4-triazol-3-amine (Intermediate F) (0.680 g, 2.98 mmol), Cs₂CO₃ (1.5 equiv.), 5-bromopicolinonitrile (0.818 g, 1.5 mmol) and 1,4-dioxane (15 mL). Vacuum was applied briefly to the reaction flask followed by backfilling with N₂ and the procedure was repeated 5 times. The mixture was then heated to 100° C. and stirred for 5 h. LCMS showed that no amine was left. The volatiles were removed in vacuo and the residue was treated with H₂O and 1N HCl to adjust pH to 4-5. The solid precipitates were collected via filtration and washed with 5% potassium xanthate (2×), H₂O (5×), diethyl ether (5×) to provide 5-((1-(4-(trifluoromethyl)phenyl)-1H-1,2,4-triazol-3-yl)amino)picolinonitrile (0.655 g, 66.54% yield). ¹H NMR (401 MHz, DMSO-d₆) δ 10.56 (s, 1H), 9.34 (s, 1H), 8.84 (d, J=2.2 Hz, 1H), 8.26 (dd, J=8.6, 2.4 Hz, 1H), 8.09 (d, J=8.4 Hz, 2H), 7.92 (d, J=8.4 Hz, 3H). ¹³C NMR (101 MHz, DMSO-d₆) δ 160.18, 142.89, 141.32, 140.36, 139.88, 130.22, 127.69, 127.53, 127.49, 127.37, 125.80, 123.10, 122.68, 122.12, 119.20, 118.80. LCMS R_{f} (min)=3.337. HRMS (ESI) calcd for C₁₅H₁₀F₃N₆⁺ [M+H]⁺ 331.093, found 331.0914

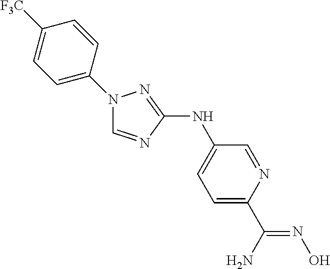 [SMILES: N/C(=N\O)c1ccc(Nc2ncn(-c3ccc(C(F)(F)F)cc3)n2)cn1]

5-((1-(4-(Trifluoromethyl)phenyl)-1H-1,2,4-triazol-3-yl)amino)picolinonitrile (0.18 g, 0.545 mmol) was suspended in EtOH (25 mL) and NH₂OH·HCl (0.303 g, 4.36 mmol) added. Approximately half of the EtOH was then removed in vacuo. Et₃N (0.595 mL, 4.36 mol) was then added and the resulting reaction mixture was stirred at reflux overnight. LCMS indicated that the reaction was complete. The volatiles were removed and the resulting solids were suspended in water, collected by filtration then washing well with H₂O. The solids were washed with Et₂O and dried to provide the title compound (0.143 g, 72.2% yield). ¹H NMR (401 MHz, DMSO-d₆) δ 10.01 (s, 1H), 9.68 (s, 1H), 9.29 (s, 1H), 8.86 (s, 1H), 8.09-7.82 (m, 6H), 5.75 (s, 2H). ¹³C NMR (101 MHz, DMSO-d₆) δ 160.94, 150.03, 142.65, 142.07, 140.06, 138.68, 136.73, 127.52, 127.48, 127.38, 127.06, 125.85, 123.62, 123.16, 120.06, 118.98. LCMS R_{f} (min)=2.924. HRMS (ESI) calcd for C₁₅H₁₃F₃N₇O⁺ [M+H]⁺ 364.1128, found 364.1140.

### 25. N-Hydroxy-N-methyl-5-((1-(4-(trifluoromethyl)phenyl)-1H-1,2,4-triazol-3-yl)amino)picolinamide (Scheme 23)

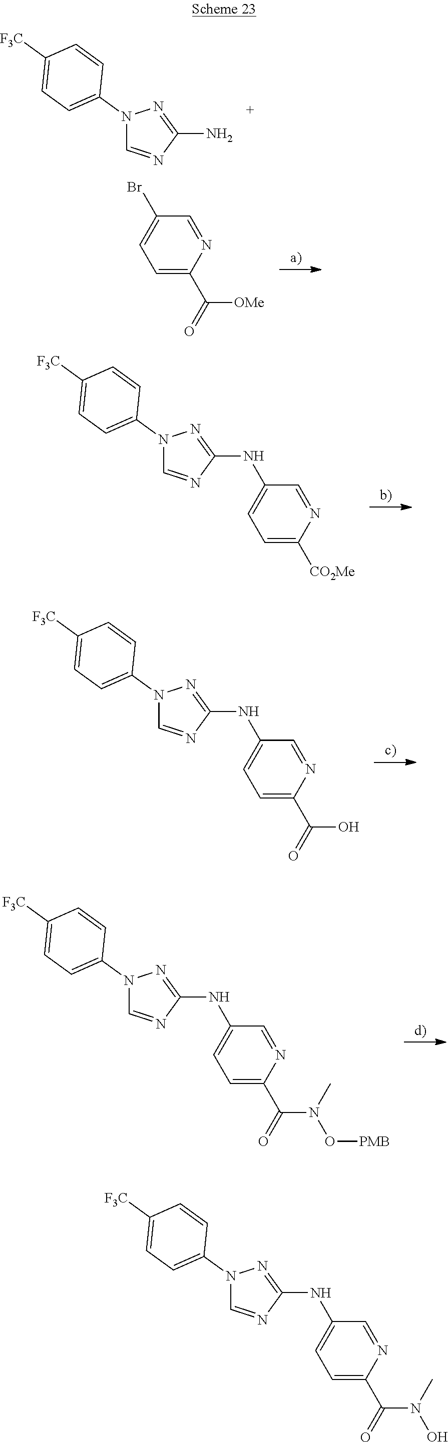

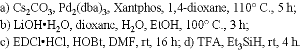

An oven-dried RBF was charged with Pd₂(dba)₃ (0.130 g, 0.05 equiv.), Xantphos (0.165 g, 0.1 equiv.), 1-(4-(trifluoromethyl)phenyl)-1H-1,2,4-triazol-3-amine (Intermediate F) (0.650 g, 2.848 mmol), Cs₂CO₃ (1.392 g, 1.5 equiv.), methyl 5-bromopicolinate (0.823 g, 1.5 mmol) and 1,4-dioxane (15 mL). Vacuum was applied briefly to the reaction flask followed by backfilling with N₂ and the procedure was repeated 5 times. The mixture was then heated to 110° C. and stirred for 5 h. LCMS showed that no amine was left. The reaction was poured into H₂O, acidified with 1N HCl (pH 4-5) then extracted with chloroform (5×). The combined organic phases were dried over MgSO₄ and evaporated to dryness. The residue was chromatographed on silica gel using mixtures of DCM/EtOAc as eluents to provide methyl 5-((1-(4-(trifluoromethyl)phenyl)-1H-1,2,4-triazol-3-yl)amino)picolinate (0.42 g, 40.6% yield). ¹H NMR (401 MHz, DMSO-d₆) δ 10.39 (s, 1H), 9.34 (s, 1H), 8.88 (d, J=2.3 Hz, 1H), 8.23 (dd, J=8.7, 2.7 Hz, 1H), 8.10 (d, J=9.0 Hz, 2H), 8.04 (d, J=8.7 Hz, 1H), 7.93 (d, J=8.6 Hz, 2H), 3.84 (s, 3H). LCMS R_{f} (min)=3.352. HRMS (ESI) calcd for C₁₆H₁₃F₃N₅O₂⁺ [M+H]⁺ 364.1016, found 364.1028

Methyl 5-((1-(4-(trifluoromethyl)phenyl)-1H-1,2,4-triazol-3-yl)amino)picolinate (0.4 g, 1.10 mmol) was mixed with LiOH·H₂O (0.138 g, 3.30 mmol), dioxane (1.8 mL), H₂O (1.1 mL) and EtOH (2.8 mL). The resulting mixture was heated to 100° C. and stirred for 3 h. All volatiles were removed in vacuo and the residue acidified with 1N HCl (pH-4). The solid was filtered, washed with H₂O (3×) and dried under high vacuum to provide 5-((1-(4-(trifluoromethyl)phenyl)-1H-1,2,4-triazol-3-yl)amino)picolinic acid (0.355 g, 92.3% yield). ¹H NMR (401 MHz, DMSO-d₆) δ 10.06 (s, 1H), 9.30 (s, 1H), 8.68 (s, 1H), 8.17 (d, J=8.2 Hz, 1H), 8.07 (d, J=8.0 Hz, 2H), 7.94 (m, 3H). LCMS R_{f} (min)=3.379, MS m/z=349.9 [M+H]⁺.

5-((1-(4-(Trifluoromethyl)phenyl)-1H-1,2,4-triazol-3-yl)amino)picolinic acid (0.3 g, 0.858 mmol) was suspended in DMF (17 mL), EDCI·HCl (0.214 g, 1.116 mmol) and HOBt (0.141 g, 1.1202 mmol) added. The resulting mixture was stirred at rt for 3 h. O-(4-methoxybenzyl)-N-methylhydroxylamine (0.287 g, 1.717 mmol) was added and the stirring continued overnight. The reaction mixture was diluted with sat. NaHCO₃ solution and EtOAc. After separation, the aq. phase was extracted with further EtOAc (3×). The combined organic phases were dried over MgSO₄ and evaporated to dryness. The residue was purified on silica gel using a mixture of petroleum spirit and EtOAc as eluents. The fractions containing the desired product were combined and evaporated to dryness. The solid residue was filtered and washed well with Et₂O to provide the product N-((4-methoxybenzyl)oxy)-N-methyl-5-((1-(4-(trifluoromethyl)phenyl)-1H-1,2,4-triazol-3-yl)amino)picolinamide (224 mg, 52.31% yield). ¹H NMR (401 MHz, DMSO-d₆) δ 10.18 (s, 1H), 9.33 (s, 1H), 8.85 (d, J=2.5 Hz, 1H), 8.19 (d6, J=8.6, 2.6 Hz, 1H), 8.12 (d, J=8.5 Hz, 2H), 7.94 (d, J=8.7 Hz, 2H), 7.64 (d, J=8.6 Hz, 1H), 7.22 (d, J=7.7 Hz, 2H), 6.87 (d, J=8.5 Hz, 2H), 4.95 (s, 2H), 3.71 (s, 3H), 3.33 (s, 3H). LCMS R_{f} (min)=3.517. HRMS (ESI) calcd for C₂₅H₂₂F₃N₄O₄⁺ [M+H]⁺ 499.1588, found 499.1578.

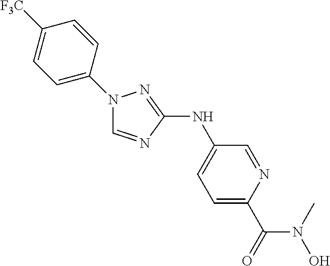 [SMILES: CN(O)C(=O)c1ccc(Nc2ncn(-c3ccc(C(F)(F)F)cc3)n2)cn1]

N-((4-Methoxybenzyl)oxy)-N-methyl-5-((1-(4-(trifluoromethyl)phenyl)-1H-1,2,4-triazol-3-yl)amino)picolinamide (0.105 g, 0.21 mmol) was stirred in a mixture of TFA (2 mL) and Et₃SiH (0.105 mL) at rt for 4 h. All volatiles were removed on rotavap and the residue filtered with toluene then washed with DCM and Et₂O. The solid was freeze-dried from dioxane then filtered with MeOH and washed with a small amount of MeOH and Et₂O to provide the title compound (0.051 g, 64.1% yield). ¹H NMR (401 MHz, DMSO-d₆) δ 10.18 (s, 1H), 9.32 (s, 1H), 8.83 (s, 1H), 8.16 (m, 1H), 8.10 (m, 2H), 7.94 (m, 2H), 7.71 (s, 1H) (the resonance of N-Me overlapped with H₂O, and not assigned). ¹³C NMR (101 MHz, DMSO-d₆) δ 142.80, 140.00, 139.66, 127.50, 127.23, 125.83, 125.25, 123.13, 120.43, 119.11, 31.14. LCMS R_{f} (min)=3.337. HRMS (ESI) calcd for C₁₆H₁₄F₃N₆O₂⁺ [M+H]⁺ 379.1125, found 379.1138.

### 26. N′,3-Dihydroxy-5-((5-(4-(trifluoromethyl)phenyl)oxazol-2-yl)amino)picolinimidamide (Scheme 24)

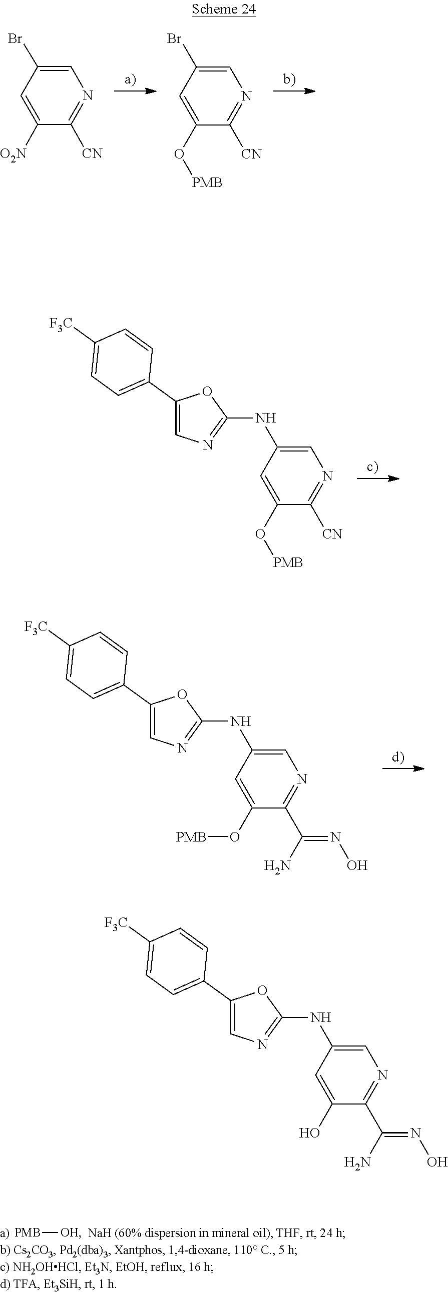

(Intermediate G—5-bromo-3-((4-methoxybenzyl)oxy)picolinonitrile) p-Methoxybenzyl alcohol (1.57 g, 11.4 mmol, 1.3 equiv.) was dissolved in anhydrous THF (30 mL), cooled to 0° C. and NaH (60%, 0.273 g, 11.4 mmol, 1.3 equiv.) added. The mixture was stirred at 0° C. for 30 min then 5-bromo-3-nitropicolinonitrile (2 g, 8.77 mmol) was added. The resultant reaction mixture turned black and was stirred at rt for 24 h. LCMS indicated that there was still some starting material, so more NaH (60%, 0.5 equiv.) was added and the stirring continued for another 24 h. The reaction mixture was poured into sat. NaHCO₃ solution (300 mL) and extracted with EtOAc (3×100 mL). The combined organic layers were dried over MgSO₄ and concentrated to dryness. The solid residue was purified on silica gel eluting with neat toluene to provide 5-bromo-3-((4-methoxybenzyl)oxy)picolinonitrile (Intermediate G) (1.02 g, 36.43% yield). ¹H NMR (401 MHz, CDCl₃) δ 8.32 (d, J=1.8 Hz, 1H), 7.55 (d, J=1.8 Hz, 1H), 7.35 (d, J=8.7 Hz, 2H), 6.93 (d, J=8.7 Hz, 2H), 5.16 (s, 2H), 3.82 (s, 3H). ¹³C NMR (101 MHz, CDCl₃) δ 160.09, 157.63, 144.06, 144.04, 129.16, 128.24, 125.99, 125.09, 123.92, 122.59, 114.58, 114.41, 71.43, 55.37. LCMS R_{f}(min)=3.337. HRMS (ESI) calcd for C₁₄H₁₂BrN₂O₂⁺ [M+H]⁺ 319.0077, found 319.0068.

An oven-dried RBF was charged with Pd₂(dba)₃ (0.064 g, 0.05 equiv.), Xantphos (0.081 g, 0.1 equiv.), 5-bromo-3-((4-methoxybenzyl)oxy)picolinonitrile (Intermediate G) (0.447 g, 1.402 mmol), Cs₂CO₃ (0.685 g, 1.5 equiv.), 5-(4-(trifluoromethyl)phenyl)oxazol-2-amine (Intermediate D) (0.32 g, 1.402 mmol) and 1,4-dioxane (7 mL). Vacuum was applied briefly to the reaction flask followed by backfilling with N₂ and the procedure was repeated 5 times. The mixture was then heated to 110° C. and stirred for 5 h. LCMS showed that starting materials had been consumed. The volatiles were removed in vacuo and the residue treated with H₂O and 1N HCl to adjust the pH to 4-5. The solid precipitates were collected via filtering and washed with 5% aqueous solution of potassium ethyl xanthate (2×), H₂O (5×), Et₂O (5×) to provide 3-((4-methoxybenzyl)oxy)-5-((5-(4-(trifluoromethyl)phenyl)oxazol-2-yl)amino)picolinonitrile (0.502 g, 76.74% yield). ¹H NMR (401 MHz, DMSO-d₆) δ 11.43 (br, 1H), 8.36 (s, 1H), 8.28 (s, 1H), 7.84 (s, 1H), 7.82 (s, 4H), 7.48 (d, J=8.4 Hz, 2H), 7.00 (d, J=8.4 Hz, 2H), 5.25 (s, 2H), 3.78 (s, 3H). ¹³C NMR (101 MHz, DMSO-d₆) δ 159.90, 159.02, 156.25, 144.08, 140.98, 133.28, 131.75, 130.44, 127.95, 127.60, 126.58, 126.00, 125.70, 123.71, 123.30, 116.55, 114.47, 113.84, 107.50, 70.65, 55.61. LCMS R_{f} (min)=3.642, MS m/z=466.9 [M+H]⁺.

3-((4-Methoxybenzyl)oxy)-5-((5-(4-(trifluoromethyl)phenyl)oxazol-2-yl)amino)picolinonitrile (0.208 g, 0.445 mmol) was suspended in EtOH (25 mL), NH₂OH·HCl (0.248 g, 3.567 mmol) added and approximately half of the EtOH was removed in vacuo. Et₃N (0.487 mL, 3.567 mol) was added and the resulting reaction mixture was stirred at reflux overnight. LCMS indicated that the reaction was complete. The volatiles were removed and the resulting solids were suspended in water, collected by filtration then washing well with H₂O. The solids were washed with Et₂O, dried in high vacuum to provide N′-hydroxy-3-((4-methoxybenzyl)oxy)-5-((5-(4-(trifluoromethyl)phenyl)oxazol-2-yl)amino)picolinimidamide (0.176 g, 79% yield). ¹H NMR (401 MHz, DMSO-d₆) δ 10.91 (s, 1H), 9.66 (s, 1H), 8.36 (s, 1H), 8.02 (s, 1H), 7.80 (s, 4H), 7.78 (s, 1H), 7.47 (d, J=8.5 Hz, 2H), 6.94 (d, J=8.6 Hz, 2H), 5.73 (s, 2H), 5.12 (s, 2H), 3.75 (s, 4H). LCMS R_{f} (min)=3.202. HRMS (ESI) calcd for C₂₄H₂₁F₃N₅O₄⁺ (M)⁺ 499.1574, found 499.1549.

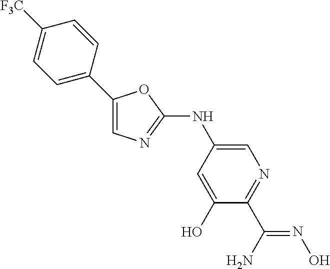 [SMILES: N/C(=N\O)c1ncc(Nc2ncc(-c3ccc(C(F)(F)F)cc3)o2)cc1O]

N′-Hydroxy-3-((4-methoxybenzyl)oxy)-5-((5-(4-(trifluoromethyl)phenyl)oxazol-2-yl)amino)picolinimidamide (0.163 g, 0.326 mmol) was stirred in a mixture of TFA (3.26 mL) and Et₃SiH (0.163 mL) at rt. LCMS indicated that the reaction was complete after 1 h. The reaction mixture was filtered to remove black precipitates, washed with more TFA (3×). The combined TFA phases were evaporated to dryness, filtered with Et₂O and then washed with toluene, DCM and Et₂O. The residue was purified on preparative HPLC to provide the title compound (0.072 g, 58.16% yield). ¹H NMR (401 MHz, DMSO-d₆) δ 11.15 (s, 1H), 10.62-10.55 (brs, 1H), 8.30 (d, J=2.1 Hz, 2H), 7.97 (s, 1H), 7.81 (s, 4H), 7.79 (s, 1H), 3.56 (s, 1H). LCMS R_{f} (min)=3.243, MS m/z=379.9 [M+H]⁺. HRMS (ESI) calcd for C₁₆H₁₃F₃N₅O₃⁺ [M+H]⁺ 380.0965, found 380.0977.

### 27. 1-Hydroxy-6-methyl-4-((5-(4-(trifluoromethyl)phenyl)oxazol-2-yl)amino)pyridin-2(1H)-one (Scheme 25)

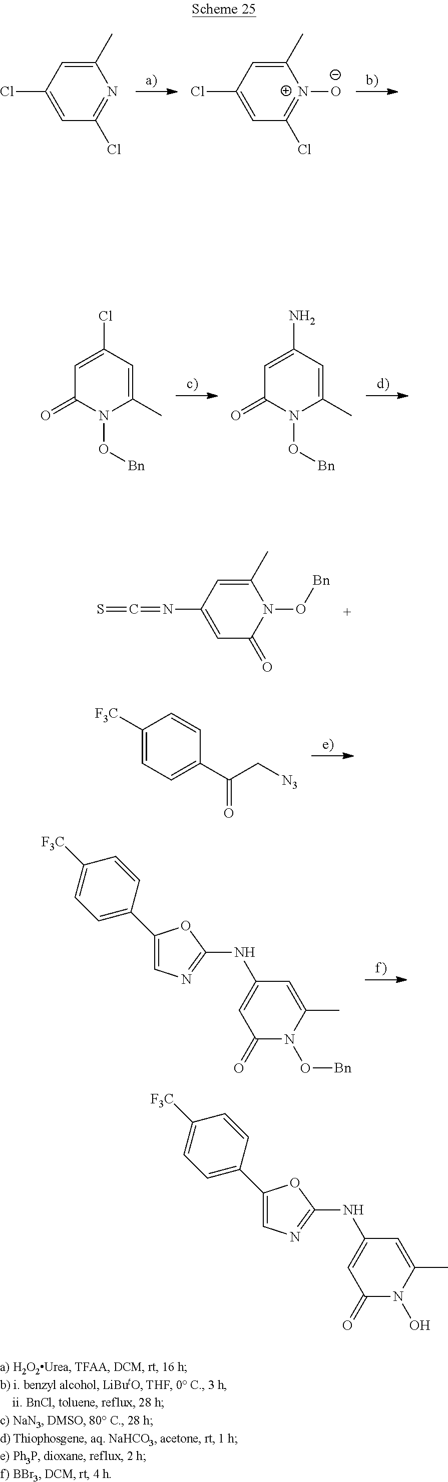

To a solution of 2,4-dichloro-6-methylpyridine (2.50 g, 15.43 mmol) in dry DCM (60 mL) was added H₂O₂·urea (2 equiv.). On cooling to 0° C., a solution of TFAA (2 equiv.) in dry DCM was added dropwise and the mixture stirred at rt overnight. The reaction mixture was diluted with sat. NaS₂O₃ (15 mL) and stirred at rt for 0.5 h then poured into H₂O (20 mL) and extracted with DCM (7×20 mL). The combined organic layers were washed with 1 M NaOH (15 mL), dried over MgSO₄ and concentrated to a creamy semi-solid. The solid was chromatographed on silica gel eluting with 5% EtOH in DCM, providing 2,4-dichloro-6-methylpyridine 1-oxide as a yellow crystalline solid (2.4 g, 87.4% yield). ¹H NMR (401 MHz, CDCl₃) δ 7.42 (dd, J=2.9, 0.5 Hz, 1H), 7.22 (dd, J=2.9, 0.5 Hz, 1H), 2.55 (s, 3H). LCMS R_{f} (min)=1.734, MS m/z=177.9/179.9 [M+H]⁺.

Benzyl alcohol (4.186 g, 38.76 mmol) was dissolved in anhydrous THF (40 mL), cooled to 0° C. and then LiBu^{t}O (1.55 g, 19.38 mmol) added. The mixture was stirred at 0° C. for 30 min and 2,4-dichloro-6-methylpyridine 1-oxide (2.3 g, 12.92 mmol) added. The resultant reaction mixture was stirred at rt for 3 h. The reaction was deemed complete by TLC (DCM/EtOAc 9/1). The reaction mixture was poured into sat. NaHCO₃ and extracted with EtOAc (7×) until no more product was detected in the extracting EtOAc phase. The combined organic phases were dried over MgSO₄ and evaporated to dryness. The residue was dissolved in toluene (800 mL), BnCl (4 mL) added and the solution heated at reflux for 28 h. A small amount of N-oxide remained unreacted. The toluene was removed and the residue was freeze-dried from H₂O/dioxane to remove excess BnOH and BnCl. The remainder was purified on silica gel with a mixture of DCM and EtOAc to afford the product, 1-(benzyloxy)-4-chloro-6-methylpyridin-2(1H)-one (1.37 g, 42.46% yield). ¹H NMR (401 MHz, CDCl₃) δ 7.49-7.42 (m, 2H), 7.42-7.34 (m, 3H), 6.61-6.55 (m, 1H), 5.96-5.85 (m, 1H), 5.25 (s, 2H), 2.15 (s, 3H). ¹³C NMR (101 MHz, CDCl₃) δ 158.54, 146.84, 145.31, 133.56, 130.03, 129.51, 128.78, 117.91, 106.37, 17.56. LCMS R_{f} (min)=3.528. HRMS (ESI) calcd for C₁₃H₁₃ClNO₂⁺ [M+H]⁺ 250.0629, found 250.0636.

1-(Benzyloxy)-4-chloro-6-methylpyridin-2(1H)-one (1.25 g, 5.0 mmol) was stirred with NaN₃ (1.50 g, 25.0 mmol, 5 equiv.) in DMSO at 80° C. for 28 h. There was still some starting material left. Both azide and amine along with other impurities were formed. The solvents were removed by freeze-drying and the solid filtered and washed with DCM. The combined filtrates were evaporated to dryness. The residue was dissolved in MeOH, dithiothreitol (DTT, 3.09 g, 20 mmol, 4 equiv.) and LiOH·H₂O (0.84 g, 20 mmol, 4 equiv.) added. The reaction mixture was stirred at rt for 2 h then poured into sat. NaHCO₃ solution and DCM. The aq. phase was extracted with more DCM and the combined organic solutions were dried over MgSO₄, evaporated to dryness. The residue was purified on silica gel using a mixture of DCM and EtOAc then DCM and MeOH to provide 4-amino-1-(benzyloxy)-6-methylpyridin-2(1H)-one (0.176 g, 15.2% yield). ¹H NMR (401 MHz, MeOH-d₄) δ 7.50-7.41 (m, 2H), 7.40-7.33 (m, 3H), 5.64 (s, 1H), 5.56 (d, J=2.6 Hz, 1H), 5.13 (s, 2H), 2.10 (s, 3H). ¹³C NMR (101 MHz, MeOH-d₄) δ 161.73, 157.20, 145.65, 134.11, 129.69, 128.88, 128.27, 98.67, 92.21, 77.67, 16.23. HRMS (ESI) calcd for C₁₃H₁₅N₂O₂⁺ [M+H]⁺ 231.1128, found 231.1132.

4-Amino-1-(benzyloxy)-6-methylpyridin-2(1H)-one (0.157 g, 0.681 mmol) was dissolved in acetone (7 mL) and sat. NaHCO₃ solution (7 mL) and cooled to 0° C. Thiophosgene (1.2 equiv.) was added dropwise and the resultant mixture was stirred at rt for 1 h. The reaction was deemed complete by TLC (neat EtOAc). The reaction mixture was poured into sat. NaHCO₃ solution and the product extracted with DCM (5×). The combined organic phases were dried over MgSO₄ and evaporated to dryness. The residue was dissolved in dioxane (4 mL), 2-azido-1-(4-(trifluoromethyl)phenyl)ethan-1-one (Intermediate A) and Ph₃P (1.25 equiv. each) were added. The resulting mixture was stirred at reflux for 2 h. All volatiles were removed and the residue triturated with DCM. The solids were filtered with DCM and washed with Et₂O to provide 1-(benzyloxy)-6-methyl-4-((5-(4-(trifluoromethyl)phenyl)oxazol-2-yl)amino)pyridin-2(1H)-one as a creamy solid (182 mg 60.47%). ¹H NMR (401 MHz, DMSO-d₆) δ 10.81-10.65 (br, 1H), 7.81 (s, 4H), 7.77 (s, 1H), 7.52 (m, J=3.5 Hz, 2H), 7.43 (m, J=3.2 Hz, 3H), 6.88 (d, J=2.5 Hz, 1H), 6.15 (s, 1H), 5.19 (s, 2H), 2.26 (s, 3H). ¹³C NMR (101 MHz, DMSO-d₆) δ 159.41, 156.22, 147.02, 146.50, 143.84, 134.75, 131.81, 130.18, 129.51, 129.00, 127.90, 127.58, 126.56, 126.00, 125.69, 123.69, 99.75, 97.51, 77.15, 17.74. LCMS R_{f} (min)=3.499, MS m/z=441.9 [M+H]⁺.

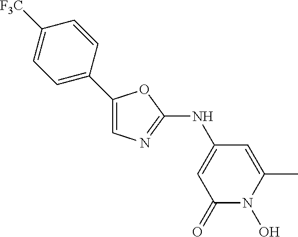 [SMILES: Cc1cc(Nc2ncc(-c3ccc(C(F)(F)F)cc3)o2)cc(=O)n1O]

1-(Benzyloxy)-6-methyl-4-((5-(4-(trifluoromethyl)phenyl)oxazol-2-yl)amino)pyridin-2(1H)-one (165 mg, 0.373 mmol) was suspended in DCM then cooled to 0° C. BBr₃ (0.142 mL; 1.495 mmol, 4 equiv.) was added dropwise. After 4 h of stirring at rt, LCMS indicated that all of SM had been consumed. Ice (5 g) added then NaHCO₃ added slowly until no gas evolved. The reaction mixture was stirred at rt for 3 h and DCM was removed in vacuo. The residue was acidified with TFA and chromatographed on preparative HPLC to provide the title compound)52 mg, 39.60%). ¹H NMR (401 MHz, DMSO-d₆) δ 10.74 (s, 1H), 7.82-7.75 (m, 4H), 7.74 (s, 1H), 6.82 (d, J=2.7 Hz, 1H), 6.24 (d, J=2.2 Hz, 1H), 2.29 (s, 3H). ¹³C NMR (101 MHz, DMSO-d₆) δ 159.13, 156.40, 146.05, 144.64, 143.68, 131.87, 127.81, 127.49, 126.56, 126.52, 126.01, 125.68, 123.60, 123.31, 98.03, 96.69, 66.82, 17.87. LCMS R_{f} (min)=3.325, MS m/z=351.9 [M+H]⁺. HRMS (ESI) calcd for C₁₆H₁₃F₃N₃O₃⁺ [M+H]⁺ 352.0904, found 352.0910.

### 28. N,3-Dihydroxy-N-methyl-5-((5-(4-(trifluoromethyl)phenyl)oxazol-2-yl)amino)picolinamide (Scheme 26)

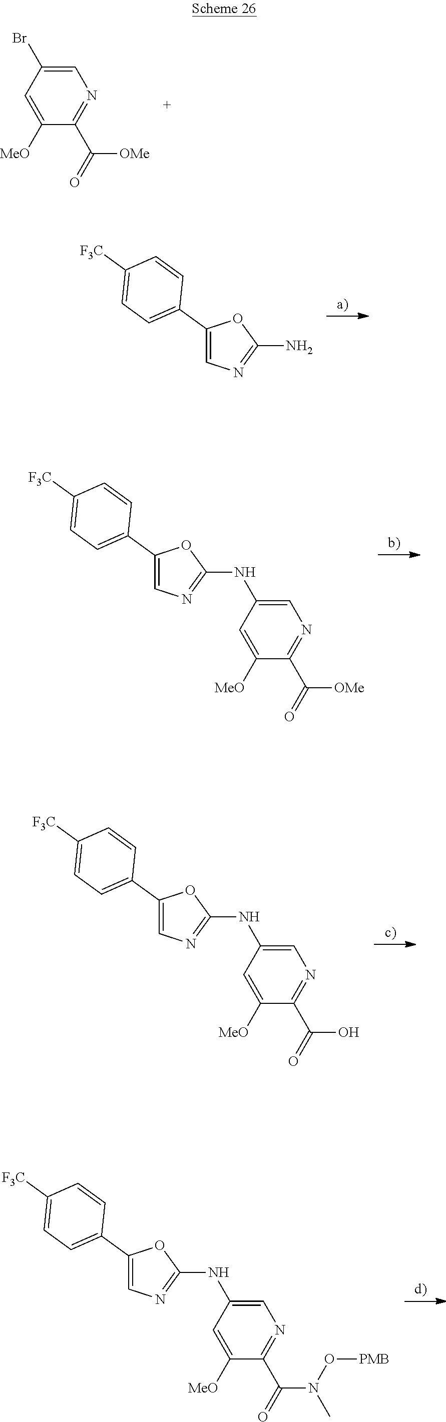

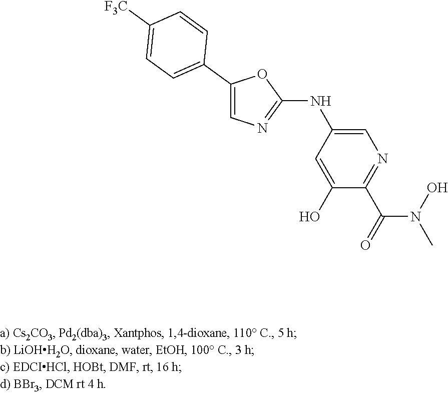 [SMILES: CN(O)C(=O)c1ncc(Nc2ncc(-c3ccc(C(F)(F)F)cc3)o2)cc1O]

5-(4-(Trifluoromethyl)phenyl)oxazol-2-amine (Intermediate D) (0.88 g, 3.576 mmol) was reacted with methyl 5-bromo-3-methoxypicolinate as per Scheme 23 (step a) to provide methyl 3-methoxy-5-((5-(4-(trifluoromethyl)phenyl)oxazol-2-yl)amino)picolinate in 82.46% yield (1.15 g). ¹H NMR (401 MHz, DMSO-d₆) δ 11.18 (s, 1H), 8.31 (s, 1H), 8.08 (s, 1H), 7.78 (s, J=5.4 Hz, 4H), 3.87 (s, 3H), 3.78 (s, 3H). ¹³C NMR (101 MHz, DMSO-d₆) δ 165.14, 156.60, 156.29, 143.79, 139.83, 131.86, 130.98, 130.14, 127.81, 127.50, 126.58, 126.01, 125.73, 123.58, 123.32, 107.23, 66.82, 56.14, 52.20. HRMS (ESI) calcd for C₁₈H₁₅F₃N₃O₄⁺ [M+H]⁺ 394.1009, found 394.1016. LCMS R_{f} (min)=3.555, MS m/z=393.9 [M+H]⁺.

Methyl 3-methoxy-5-((5-(4 (trifluoromethyl)phenyl)oxazol-2-yl)amino)picolinate (1.13 g, 2.872 mmol) was hydrolysed as per Scheme 23 (step b) to afford 3-methoxy-5-((5-(4-(trifluoromethyl)phenyl)oxazol-2-yl)amino)picolinic acid in 94.5% yield (1.03 g). ¹H NMR (401 MHz, DMSO-d₆) δ 12.44 (br, 1H), 11.12 (s, 1H), 8.32 (s, 1H), 8.06 (s, 1H), 7.80 (s, 5H), 3.87 (s, 3H). ¹³C NMR (101 MHz, DMSO-d₆) δ 165.87, 156.66, 156.10, 143.75, 139.62, 131.99, 131.87, 129.74, 127.79, 127.48, 126.57, 126.53, 126.01, 125.72, 123.56, 123.31, 107.39, 56.08. LCMS R_{f}(min)=4.057, MS m/z=379.9 [M+H]⁺.

3-Methoxy-5-((5-(4-(trifluoromethyl)phenyl)oxazol-2-yl)amino)picolinic acid (0.5 g, 1.318 mmol) was couple with O-(4-methoxybenzyl)-N-methylhydroxylamine hydrochloride as per Scheme 23 (step c) to provide the product (3-methoxy-N-((4-methoxybenzyl)oxy)-N-methyl-5-((5-(4-(trifluoromethyl)phenyl)oxazol-2-yl)amino)picolinamide) in 85.1% yield (0.593 g). ¹H NMR (401 MHz, DMSO-d₆) δ 10.96 (s, 1H), 8.34 (t, J=7.2 Hz, 1H), 8.00 (s, 1H), 7.81 (s, 4H), 7.77 (d, J=8.5 Hz, 1H), 6.93-6.83 (m, 2H), 6.79 (br, 2H), 4.71 (s, 2H), 3.81 (s, 3H), 3.67 (s, 3H), 3.32 (d, J=9.8 Hz, 3H). ¹³C NMR (101 MHz, DMSO-d₆) δ 159.92, 157.02, 152.95, 143.55, 137.83, 131.97, 131.07, 127.69, 127.37, 126.58, 126.54, 126.03, 125.80, 123.48, 123.34, 114.16, 107.28, 56.04, 55.51. LCMS R_{f} (min)=3.650. HRMS (ESI) calcd for C₂₆H₂₄F₃N₄O₅⁺ [M+H]⁺ 529.1693, found 529.1699.

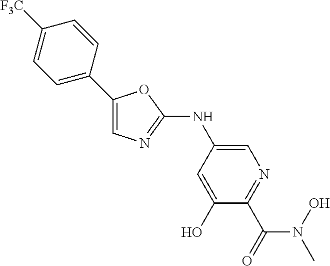 [SMILES: CN(O)C(=O)c1ncc(Nc2ncc(-c3ccc(C(F)(F)F)cc3)o2)cc1O]

3-Methoxy-N-((4-methoxybenzyl)oxy)-N-methyl-5-((5-(4-(trifluoromethyl)phenyl)-oxazol-2-yl)amino)picolinamide (193 mg, 0.365 mmol) was suspended in DCM then cooled to 0° C. BBr₃ (0.173 mL; 1.825 mmol, 5 equiv.) was added and the resultant mixture stirred at rt for 4 h. Reaction was deemed complete by LCMS. The reaction was quenched with 1.5 mL of sat. NaHCO₃ solution and solid NaHCO₃ added until gas evolution ceased. The mixture was stirred for 2 h at rt then DCM was removed in vacuo. The residual suspension was acidified with TFA and purified on prep HPLC. After evaporation to dryness, the title compound was collected by filtered with H₂O and washed with H₂O and Et₂O (73 mg, 50.69% yield). ¹H NMR (401 MHz, DMSO-d₆) δ 11.56 (s, 1H), 11.02 (s, 1H), 8.20 (d, J=1.9 Hz, 1H), 7.87 (d, J=1.8 Hz, 1H), 7.79 (s, 4H), 7.75 (d, J=7.8 Hz, 1H), 3.34 (s, 3H). ¹³C NMR (101 MHz, DMSO-d₆) δ 163.95, 156.71, 155.16, 143.67, 138.78, 131.91, 127.72, 127.41, 126.55, 126.51, 126.02, 125.76, 123.51, 123.32, 110.94. LCMS R_{f} (min)=3.904. HRMS (ESI) calcd for C₁₇H₁₄F₃N₄O₄⁺ [M+H]⁺ 395.0962, found 395.0969.

### 29. 6-((5-(4-(Trifluoromethyl)phenyl)oxazol-2-yl)amino)pyridine-3,4-diol (Scheme 27)

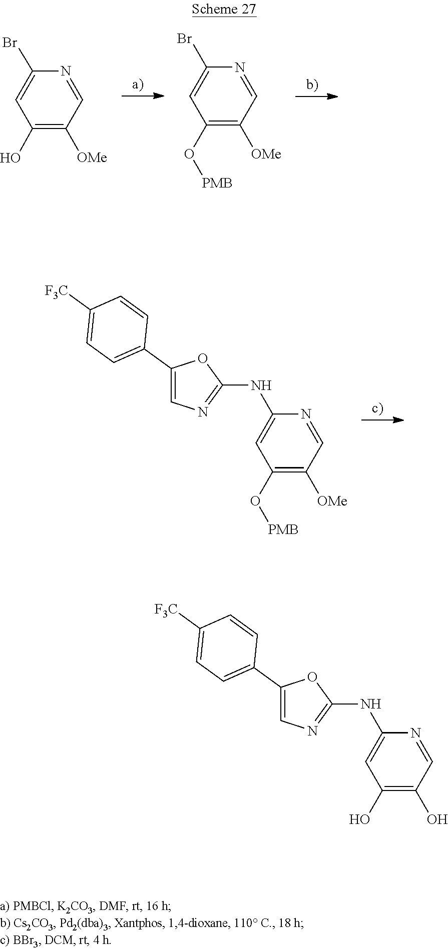

2-Bromo-5-methoxypyridin-4-ol (0.35 g, 1.715 mmol) and K₂CO₃ (0.711 g, 5.15 mmol) were suspended in DMF then PMB-Cl (0.403 g, 2.57 mmol) added. The resultant mixture was stirred at rt overnight. The reaction was deemed complete by TLC (DCM/EtOAc 9:1). The reaction mixture was poured into sat. NaHCO₃ solution and the product extracted with EtOAc (5×). The combined organic phases were dried over MgSO₄ and evaporated to dryness. The residue was purified on silica gel using a mixture of DCM and EtOAc as eluents to provide 2-bromo-5-methoxy-4-((4-methoxybenzyl)oxy)pyridine (0.356 g, 64.01% yield). ¹H NMR (401 MHz, CDCl₃) δ 7.85 (s, 1H), 7.34 (d, J=8.7 Hz, 2H), 6.97 (s, 1H), 6.92 (d, J=8.7 Hz, 2H), 5.06 (s, 2H), 3.88 (s, 3H), 3.81 (s, 3H). ¹³C NMR (101 MHz, CDCl₃) δ 159.92, 155.84, 146.23, 133.58, 133.07, 129.39, 126.89, 114.25, 111.79, 70.80, 56.76, 55.34. LCMS R_{f} (min)=3.775, MS m/z=345.9/347.9 [M+Na]⁺.

An oven-dried RBF was charged with Pd₂(dba)₃ (0.090 g, 0.10 equiv.), Xantphos (0.114 g, 0.2 equiv.), 5-(4-(trifluoromethyl)phenyl)oxazol-2-amine (Intermediate D) (0.281 g, 1.233 mmol, 1.25 equiv.), Cs₂CO₃ (0.482 g, 1.5 equiv.), 2-bromo-5-methoxy-4-((4-methoxybenzyl)oxy)pyridine (0.32 g, 0.987 mmol) and 1,4-dioxane (5 mL). Vacuum was applied briefly to the reaction flask followed by backfilling with N₂ and the procedure was repeated 5 times. The mixture was then heated to 110° C. and stirred for 18 h. The reaction mixture was poured into H₂O, acidify with 1N HCl (pH 4-5), extracted with EtOAc (5×). The combined organic phases were dried over MgSO₄, evaporated to dryness. The residue was chromatographed on silica gel using mixtures of DCM/EtOAc as eluents to provide N-(5-methoxy-4-((4-methoxybenzyl)oxy)pyridin-2-yl)-5-(4-(trifluoromethyl)phenyl)oxazol-2-amine (0.083 g, 17.83% yield). ¹H NMR (401 MHz, DMSO-d₆) δ 10.83 (s, 1H), 7.95-7.85 (br, 2H), 7.82-7.76 (m, 4H), 7.75 (s, 1H), 7.45 (d, J=8.7 Hz, 2H), 6.97 (d, J=8.7 Hz, 2H), 5.11 (s, 2H), 3.78 (s, 3H), 3.76 (s, 3H). LCMS R_{f} (min)=3.619. HRMS (ESI) calcd for C₂₄H₂₁F₃N₃O₄⁺ [M+H]⁺ 472.1479, found 472.1473.

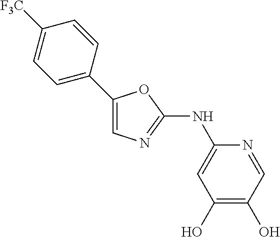 [SMILES: Oc1cnc(Nc2ncc(-c3ccc(C(F)(F)F)cc3)o2)cc1O]

N-(5-Methoxy-4-((4-methoxybenzyl)oxy)pyridin-2-yl)-5-(4-(trifluoromethyl)phenyl)oxazol-2-amine (0.077 g, 0.163 mmol) was suspended in DCM and cooled to 0° C. BBr₃ (4 equiv.) was added dropwise. After 4 h of stirring at rt, LCMS indicated that all starting material had been consumed. The volatiles were removed and the residue quenched with sat. NaHCO₃ solution and MeOH for 1 h. The volatiles were removed and the residue chromatographed on preparative HPLC to provide the title compound (0.042 g, 76.24% yield). ¹H NMR (401 MHz, DMSO-d₆) δ 7.87 (s, 1H), 7.82 (s, 5H), 7.73 (s, 1H), 7.15 (s, 1H). ¹³C NMR (101 MHz, DMSO-d₆) δ 155.76, 143.66, 140.34, 131.60, 128.06, 127.74, 126.56, 126.52, 125.98, 123.71, 123.28, 99.42. LCMS R_{f} (min)=3.333. HRMS (ESI) calcd for C₁₅H₁₁F₃N₃O₃⁺ [M+H]⁺338.0747, found 338.0751.

### 30. 3-Hydroxy-4-((5-((5-(4-(trifluoromethyl)phenyl)oxazol-2-yl)amino)pyridin-2-yl)amino)cyclobut-3-ene-1,2-dione (Scheme 28)

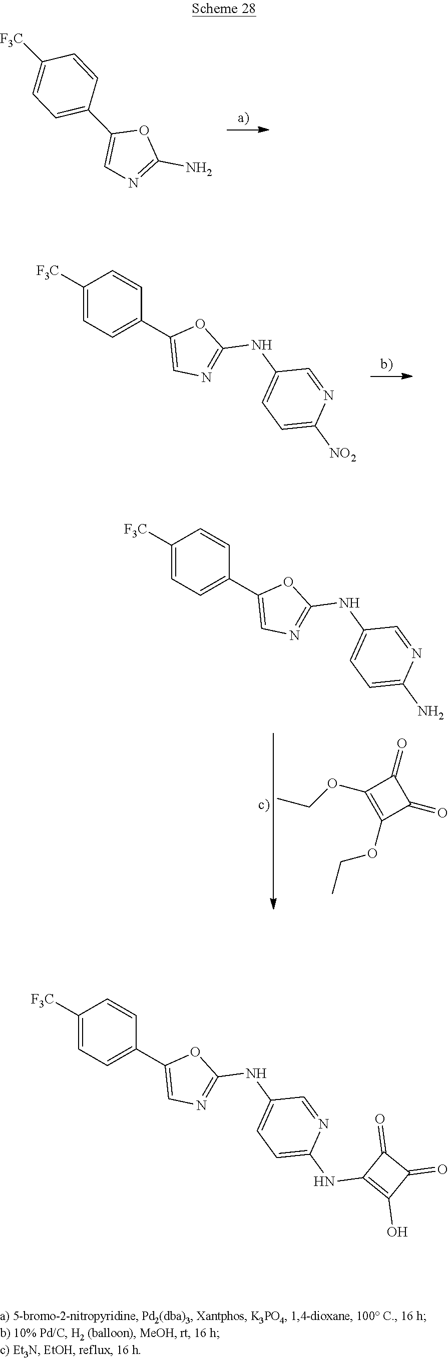

A re-sealable Schlenk tube was charged with Pd₂(dba)₃ (0.05 equiv.), Xantphos (0.1 equiv.), 5-(4-(trifluoromethyl)phenyl)oxazol-2-amine (Intermediate D) (1.2 equiv.), K₃PO₄ (1.4 equiv.), 5-bromo-2-nitropyridine (0.102 g) and 1,4-dioxane (3.0 mL). After the mixture was degassed and carefully subjected to three cycles of evacuation and backfilling with N₂, reaction mixture was heated at 100° C. overnight under N₂ atmosphere. The mixture was cooled, diluted with EtOAc (50 mL) and washed with brine (20 mL). The organic layer was separated, dried (MgSO₄), filtered and concentrated under reduced pressure to obtain the crude product, which was purified on a SiO₂ column using a Reveleris X2 flash chromatography system (EtOAc:petroleum spirit=1:1) to obtain N-(6-nitropyridin-3-yl)-5-(4-(trifluoromethyl)phenyl)oxazol-2-amine as a brown solid (130 mg, 74% yield). ¹H NMR (401 MHz, DMSO-d₆) δ 11.63 (s, 1H), 8.75 (d, J=2.5 Hz, 1H), 8.48 (dd, J=9.0, 2.6 Hz, 1H), 8.40 (d, J=9.0 Hz, 1H), 7.97-7.74 (m, 5H).

(Intermediate H—N5-(5-(4-(trifluoromethyl)phenyl)oxazol-2-yl)pyridine-2,5-diamine) To a solution of N-(6-nitropyridin-3-yl)-5-(4-(trifluoromethyl)phenyl)oxazol-2-amine (0.11 g) in MeOH (5.0 mL) was added 10% Pd/C (0.15 g). The mixture was stirred under a hydrogen atmosphere (balloon) overnight. After completion, the reaction mixture was filtered through a pad of celite and the filtrate was concentrated under reduced pressure to obtain the crude product, which was purified on a SiO₂ column (MeOH:DCM=0.05-1:10) to obtain N5-(5-(4-(trifluoromethyl)phenyl)oxazol-2-yl)pyridine-2,5-diamine as a light brown solid (70 mg, 70% yield). ¹H NMR (401 MHz, DMSO-d₆) δ 9.97 (s, 1H), 8.15 (d, J=2.4 Hz, 1H), 7.78-7.72 (m, 4H), 7.66 (dd, J=8.8, 2.8 Hz, 1H), 7.63 (s, 1H), 6.48 (d, J=8.8 Hz, 1H), 5.67 (s, 2H). ¹³C NMR (101 MHz, DMSO-d₆) δ 158.2, 155.7, 142.2, 137.8, 131.9, 128.6, 126.6, 126.3, 126.03, 126.0, 125.7, 125.6, 122.9, 122.5, 107.9. LCMS R_{f} (min)=3.04. HRMS (ESI) calcd for C₁₅H₁₂F₃N₄O⁺ [M+H]⁺ 321.0958, found 321.0966.

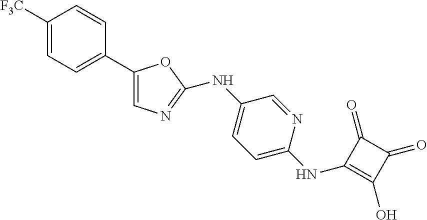 [SMILES: O=c1c(O)c(Nc2ccc(Nc3ncc(-c4ccc(C(F)(F)F)cc4)o3)cn2)c1=O]

N⁵-(5-(4-(Trifluoromethyl)phenyl)oxazol-2-yl)pyridine-2,5-diamine (0.080 g, 0.249 mmol) was stirred with 3,4-diethoxycyclobut-3-ene-1,2-dione (0.064 g, 0.374 mmol) in EtOH (3 mL) and Et₃N (0.041 mL, 0.299 mmol) at reflux overnight. The solvents were removed in vacuo and the residual mixture was purified using preparative HPLC to provide the title compound (0.073 g, 70.2% yield). ¹H NMR (401 MHz, DMSO-d₆) δ 11.50 (s, 1H), 10.94 (s, 1H), 8.72 (s, 1H), 8.17 (d, J=7.3 Hz, 1H), 7.78 (s, 4H), 7.74 (s, 1H), 7.39 (d, J=7.3 Hz, 1H). LCMS R_{f} (min)=3.672. HRMS (ESI) calcd for C₁₉H₁₂F₃N₄O₄⁺ [M+H]⁺ 417.0805, found 417.0805.

### 31. N′,3-Dihydroxy-5-((1-(4-(trifluoromethyl)phenyl)-1H-1,2,4-triazol-3-yl)amino)picolinimidamide (Scheme 29)

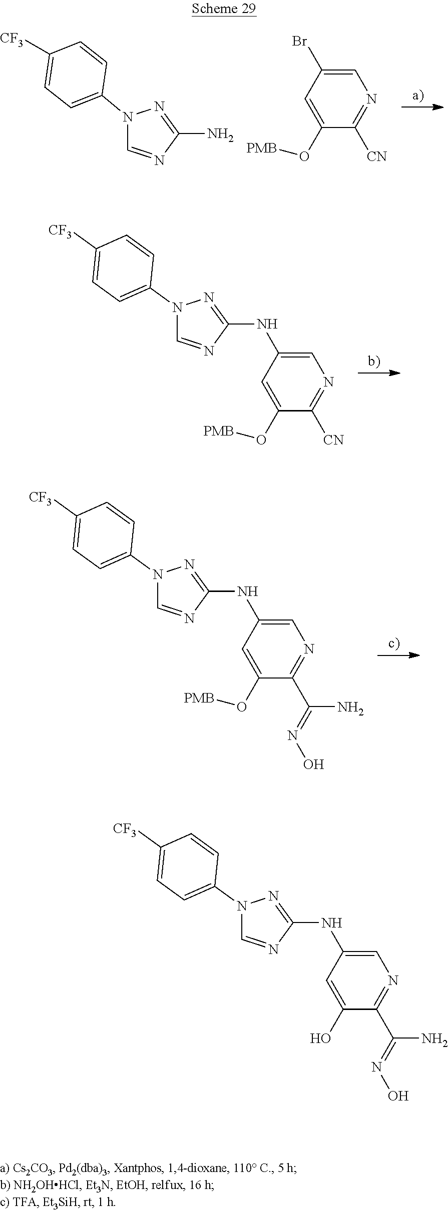

1-(4-(Trifluoromethyl)phenyl)-1H-1,2,4-triazol-3-amine (Intermediate F) (0.3 g, 1.314 mmol) was reacted with 5-bromo-3-((4-methoxybenzyl)oxy)picolinonitrile (Intermediate G) (0.419 g, 1.314 mmol) as per Scheme 24 (step b) to provide the product, 3-((4-methoxybenzyl)oxy)-5-((1-(4-(trifluoromethyl)phenyl)-1H-1,2,4-triazol-3-yl)amino)picolinonitrile in 80.4% yield (0.493 g). ¹H NMR (401 MHz, DMSO-d₆) δ 10.60 (s, 1H), 9.35 (s, 1H), 8.37 (s, 1H), 8.11 (s, 1H), 8.06 (d, J=7.5 Hz, 2H), 7.93 (d, J=7.7 Hz, 2H), 7.44 (d, J=7.6 Hz, 2H), 6.95 (d, J=7.7 Hz, 2H), 5.27 (s, 2H), 3.74 (s, 3H). ¹³C NMR (101 MHz, DMSO-d₆) δ 160.20, 159.77, 159.11, 142.79, 139.85, 133.15, 130.05, 127.83, 127.73, 127.54, 127.50, 125.79, 123.09, 119.24, 116.87, 114.44, 112.35, 106.44, 70.39, 66.82, 55.55. LCMS R_{f} (min)=3.484, MS m/z=467.0 [M+H]⁺.

3-((4-Methoxybenzyl)oxy)-5-((1-(4-(trifluoromethyl)phenyl)-1H-1,2,4-triazol-3-yl)amino)picolinonitrile (0.471 g, 1.01 mmol) was reacted as per Scheme 24 (step c) to provide N′-hydroxy-3-((4-methoxybenzyl)oxy)-5-((1-(4-(trifluoromethyl)phenyl)-1H-1,2,4-triazol-3-yl)amino)picolinimidamide in 93% yield (0.469 g). ¹H NMR (401 MHz, DMSO-d₆) δ 10.00 (s, 1H), 9.59 (s, 1H), 9.29 (s, 1H), 8.42 (s, 1H), 8.04 (s, 2H), 7.92 (s, 3H), 7.45 (s, 2H), 6.92 (s, 2H), 5.59 (s, 2H), 5.17 (s, 2H), 3.72 (s, 3H). ¹³C NMR (101 MHz, DMSO-d₆) δ 160.90, 159.27, 153.65, 150.45, 142.67, 140.02, 139.28, 132.66, 129.58, 129.29, 129.08, 127.53, 127.50, 127.41, 127.09, 125.85, 123.15, 118.97, 114.23, 108.94, 69.87, 55.48. LCMS R_{f} (min)=3.067. HRMS (ESI) calcd for C₂₃H₂₁F₃N₇O₃⁺ [M+H]⁺, 500.1652 found 500.163.

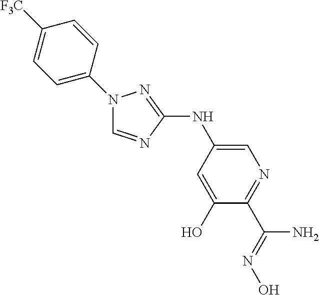 [SMILES: N/C(=N\O)c1ncc(Nc2ncn(-c3ccc(C(F)(F)F)cc3)n2)cc1O]

N′-hydroxy-3-((4-methoxybenzyl)oxy)-5-((1-(4-(trifluoromethyl)phenyl)-1H-1,2,4-triazol-3-yl)amino)picolinimidamide (0.45 g, 0.90 mmol) was deprotected as per Scheme 24 (step d) to provide the title compound in 38.33% yield (0.131 g). ¹H NMR (401 MHz, DMSO-d₆) δ 10.43 (brs, 1H), 10.22 (s, 1H), 9.33 (s, 1H), 8.34 (s, 1H), 8.10 (d, J=7.8 Hz, 2H), 7.94 (d, J=7.5 Hz, 2H), 7.88 (s, 1H), 7.19 (brs, 2H). ¹³C NMR (101 MHz, DMSO-d₆) δ 160.65, 155.36, 154.95, 142.72, 141.06, 140.01, 130.26, 127.55, 127.18, 125.83, 123.13, 118.99, 109.80. LCMS R_{f} (min)=3.072. HRMS (ESI) calcd for C₁₅H₁₃F₃N₇O₂⁺ [M+H]⁺, 380.1077 found 380.1085.

### 32. N-Ethyl-N-hydroxy-5-((5-(4-(trifluoromethyl)phenyl)oxazol-2-yl)amino)picolinamide (Scheme 30)

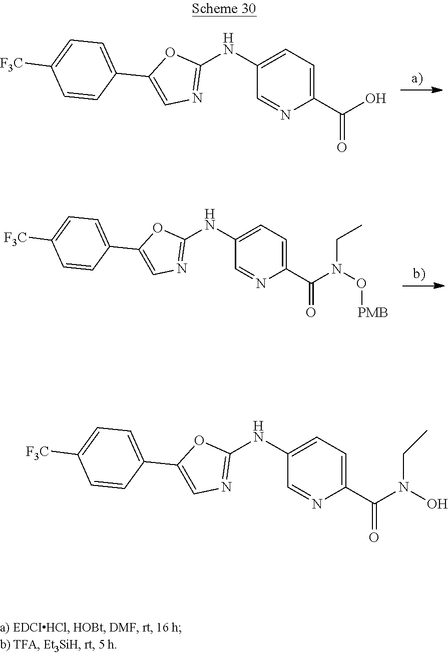

5-((5-(4-(Trifluoromethyl)phenyl)oxazol-2-yl)amino)picolinic acid (Intermediate B) (0.31 g, 0.89 mmol) was added to a solution of HOBt (0.12 g, 1.07 mmol) and EDCI·HCl (0.19 g, 0.98 mmol) in anhydrous DMF (15 mL). The resulting mixture was stirred at rt for 3 h under N₂. N-Ethyl-O-(4-methoxybenzyl)hydroxylamine (0.19 g, 1.07 mmol) was then added to the mixture and allowed to stir for a further 16 h. The reaction mixture was quenched with aq. NaHCO₃ solution (20 mL) and diluted with EtOAc (20 mL). The organic layer was extracted with EtOAc (3×25 mL). The combined organic fractions were dried over MgSO₄, filtered and concentrated under reduced pressure. The residue was chromatographed on silica gel eluting with 50% EtOAc in DCM to afford N-ethyl-N-((4-methoxybenzyl)oxy)-5-((5-(4-(trifluoromethyl)phenyl)oxazol-2-yl)amino)picolinamide as an off-white solid (0.243 g, 53% yield). ¹H NMR (400 MHz, DMSO-d₆) δ 11.04 (s, 1H), 8.81 (d, J=2.4 Hz, 1H), 8.24 (dd, J=8.6, 2.6 Hz, 1H), 7.80 (s, 4H), 7.77 (s, 1H), 7.66 (d, J=8.1 Hz, 1H), 7.20 (d, J=8.3 Hz, 2H), 6.88 (d, J=8.5 Hz, 2H), 4.92 (s, 2H), 3.77 (q, J=7.0 Hz, 2H), 3.72 (s, 3H), 1.18 (t, J=7.0 Hz, 3H). ¹³C NMR (100 MHz, DMSO-d₆) δ 159.90, 156.87, 146.18, 143.66, 137.55, 137.39, 131.96, 131.28, 127.70, 127.39, 126.03, 125.79, 123.77, 123.50, 123.33, 114.16, 75.62, 67.86, 55.58, 55.54, 40.00. LCMS R_{f}(min)=3.65, MS m/z 513.0 [M+H]⁺.

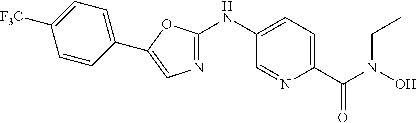 [SMILES: CCN(O)C(=O)c1ccc(Nc2ncc(-c3ccc(C(F)(F)F)cc3)o2)cn1]

N-Ethyl-N-((4-methoxybenzyl)oxy)-5-((5-(4-(trifluoromethyl)phenyl)oxazol-2-yl)amino)picolinamide (0.20 g, 0.39 mmol) was dissolved in TFA (4.0 mL) and Et₃SiH (0.4 mL) and the mixture was stirred at rt for 5 h. The volatiles were then removed under reduced pressure and the residue was directly subjected to preparative HPLC using 0.1% TFA in H₂O and 0.1% TFA in ACN as eluents to yield the title compound as bright yellow solid (0.14 g, 92%). ¹H NMR (400 MHz, DMSO-d₆) δ 11.04 (s, 1H), 8.79 (s, 1H), 8.24 (dd, J=8.6, 2.4 Hz, 1H), 7.80 (s, 4H), 7.76 (s, 1H), 7.71 (d, J=7.8 Hz, 1H), 3.71 (dd, J=13.6, 6.7 Hz, 2H), 1.19 (t, J=7.0 Hz, 3H). ¹³C NMR (100 MHz, DMSO-d₆) δ 156.84, 143.66, 137.19, 131.94, 128.82, 128.72, 127.71, 127.39, 126.02, 125.77, 124.01, 123.51, 69.00, 40.89. LCMS R_{f} (min)=3.68, MS m/z 393.9 [M+H]⁺. HRMS (ESI) calcd for C₁₈H₁₆F₃N₄O₃⁺ [M+H]⁺ 393.1169, found 393.1172.

### 33. N-Hydroxy-N-propyl-5-((5-(4-(trifluoromethyl)phenyl)oxazol-2-yl)amino)picolinamide (Scheme 31)

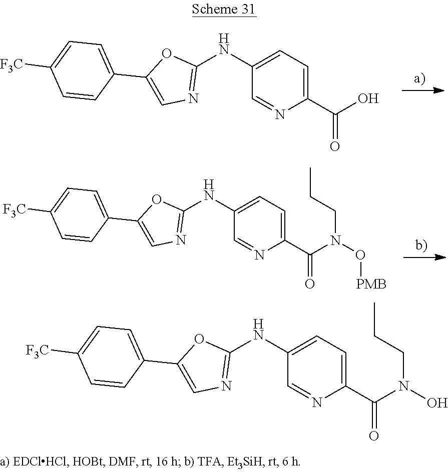

5-((5-(4-(Trifluoromethyl)phenyl)oxazol-2-yl)amino)picolinic acid (Intermediate B) (0.132 g, 0.38 mmol) HOBt (0.053 g, 0.45 mmol) and EDCl·HCl (0.080 g, 0.42 mmol) were added into a dry RBF and stirred with anhydrous DMF (6.5 mL) for 3 h with continuous flow of N₂. N-Propyl-O-(4-methoxybenzyl)hydroxylamine (0.089 g, 0.45 mmol) was then added to the reaction mixture and stirring was continued for 16 h. After this period of time the reaction was quenched with aq. NaHCO₃ solution (10 mL) and diluted with EtOAc (10 mL). The organic layer was extracted with EtOAc (3×10 mL) and the combined organics were dried over MgSO₄. The mixture was filtered, concentrated under reduced pressure and the crude material (0.112 g) was used in the next step without further purification. LCMS R_{f} (min)=3.69, MS m/z 527.0 [M+H]⁺.

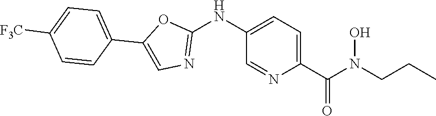 [SMILES: CCCN(O)C(=O)c1ccc(Nc2ncc(-c3ccc(C(F)(F)F)cc3)o2)cn1]

TFA (2.5 mL) and Et₃SiH (0.3 mL) were combined with crude N-((4-methoxybenzyl)oxy)-N-propyl-5-((5-(4-(trifluoromethyl)phenyl)oxazol-2-yl)amino)picolinamide (0.112 g) and the resulting mixture was stirred at rt for 6 h. The volatiles were removed under reduced pressure and the residue was directly subjected to preparative HPLC using 0.1% TFA in H₂O and 0.1% TFA in ACN as eluents to yield the title compound as bright yellow solid (0.082 g, 54% over two steps). ¹H NMR (400 MHz, DMSO-d₆) δ 11.01 (s, 1H), 8.78 (d, J=2.4 Hz, 1H), 8.23 (dd, J=8.6, 2.3 Hz, 1H), 7.80 (s, 4H), 7.77 (s, 1H), 7.68 (d, J=7.5 Hz, 1H), 3.66 (t, J=6.0 Hz, 2H), 1.73-1.54 (m, 2H), 0.86 (t, J=8.0 Hz, 3H). ¹³C NMR (100 MHz, DMSO-d₆) δ 156.88, 143.63, 137.28, 137.10, 132.15, 131.95, 127.70, 127.38, 126.03, 125.79, 123.50, 66.82, 11.44. LCMS R_{f} (min)=3.39, MS m/z 407.0 [M+H]⁺. HRMS (ESI) calcd for C₁₉H₁₈F₃N₄O₃⁺ [M+H]⁺ 407.1326, found 407.1336.

### 34. N-Hydroxy-N-isopropyl-5-((5-(4-(trifluoromethyl)phenyl)oxazol-2-yl)amino)picolinamide (Scheme 32)

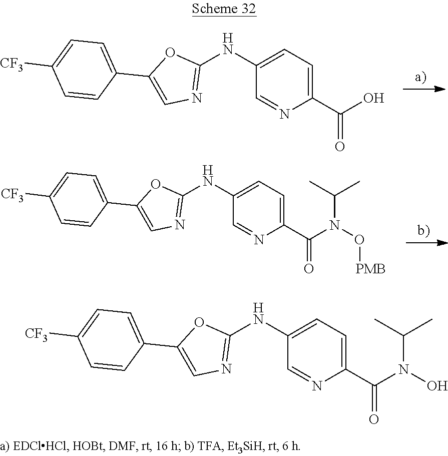

5-((5-(4-(Trifluoromethyl)phenyl)oxazol-2-yl)amino)picolinic acid (0.086 g) was converted to N-isopropyl-N-((4-methoxybenzyl)oxy)-5-((5-(4-(trifluoromethyl)phenyl)oxazol-2-yl)amino)picolinamide as per Scheme 31 (step a). The crude product used in the next step without further purification. LCMS R_{f} (min)=3.51, MS m/z=527.0 (M+H)⁺.

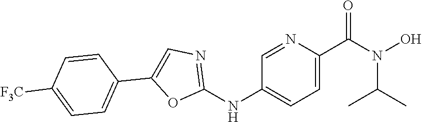 [SMILES: CC(C)N(O)C(=O)c1ccc(Nc2ncc(-c3ccc(C(F)(F)F)cc3)o2)cn1]

TFA (2.0 mL) and Et₃SiH (0.2 mL) were added into a RBF with the crude N-isopropyl-N-((4-methoxybenzyl)oxy)-5-((5-(4-(trifluoromethyl)phenyl)oxazol-2-yl)amino)picolinamide and the resulting mixture was stirred at rt for 6 h. The volatiles were removed under reduced pressure and the residue was directly subjected to preparative HPLC using 0.1% TFA in H₂O and 0.1% TFA in ACN as eluents to yield N-hydroxy-N-isopropyl-5-((5-(4-(trifluoromethyl)phenyl)oxazol-2-yl)amino)picolinamide as bright yellow solid (0.064 g, 37% over two steps). ¹H NMR (400 MHz, DMSO-d₆) δ 11.02 (s, 1H), 8.79 (s, 1H), 8.23 (dd, J=8.6, 2.4 Hz, 1H), 7.80 (s, 4H), 7.76 (s, 1H), 7.67 (d, J=8.5 Hz, 1H), 4.58 (s, 1H), 1.17 (d, J=6.6 Hz, 6H). ¹³C NMR (100 MHz, DMSO) δ 156.70, 143.45, 131.78, 127.52, 127.20, 126.39, 126.35, 125.85, 125.61, 123.32, 123.15, 40.72, 39.52. LCMS R_{f} (min)=3.37, MS m/z 407.0 (M+H)⁺. HRMS (ESI) calcd for C₁₉H₁₈F₃N₄O₃⁺ (M+H)⁺407.1326, found 407.1338.

### 35. 1-Hydroxy-1-methyl-3-(5-((5-(4-(trifluoromethyl)phenyl)oxazol-2-yl)amino)pyridin-2-yl)urea (Scheme 33)

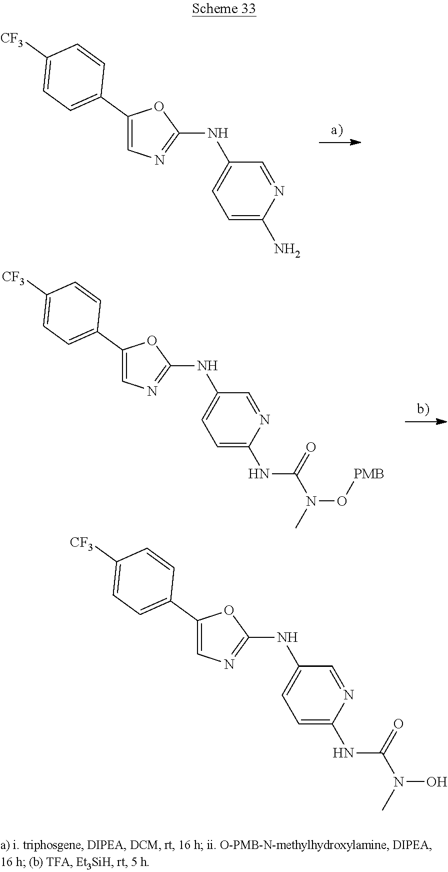

N⁵-(5-(4-(Trifluoromethyl)phenyl)oxazol-2-yl)pyridine-2,5-diamine (Intermediate H) (0.1 g, 0.312 mmol) was suspended in dry DCM (6 mL), and triphosgene (0.037 g, 0.124 mmol) and DIPEA (0.080 g, 0.624 mmol) were added. The resulting mixture was stirred overnight at rt. 0-(4-Methoxybenzyl)-N-methylhydroxylamine and DIPEA (2 equiv. each) were added and the reaction was stirred at rt overnight. The reaction mixture was quenched with sat. NaHCO₃ (aq.) and was extracted with EtOAc (3×). The organic solutions were combined, dried over MgSO₄ and evaporated to dryness. The residue was chromatographed on silica gel with neat EtOAc to provide 1-((4-methoxybenzyl)oxy)-1-methyl-3-(5-((5-(4-(trifluoromethyl)phenyl)oxazol-2-yl)amino)pyridin-2-yl)urea (34 mg, 21.2% yield). ¹H NMR (400 MHz, CDCl₃) δ 8.65 (s, 1H), 8.12 (d, J=8.9 Hz, 1H), 7.56 (s, 1H), 7.49 (s, 4H), 7.33-7.29 (m, 2H), 7.23 (d, J=8.7 Hz, 1H), 6.75 (d, J=8.2 Hz, 2H), 4.83 (s, 2H), 3.65 (s, 3H), 2.97 (s, 3H). ¹³C NMR (100 MHz, DMSO-d₆) δ 159.42, 156.39, 145.71, 143.18, 137.07, 136.92, 131.48, 130.80, 128.65, 128.25, 127.23, 126.91, 125.55, 125.31, 123.30, 123.03, 122.85, 113.69, 75.15, 55.06, 39.52, 33.23. LCMS R_{f} (min)=4.09, MS m/z 514.1 [M+H]⁺. HRMS (ESI) calcd for C₂₅H₂₃F₃N₅O₄⁺ [M+H]⁺ 514.1697, found 514.1683.

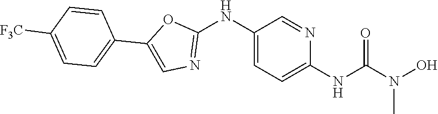 [SMILES: CN(O)C(=O)Nc1ccc(Nc2ncc(-c3ccc(C(F)(F)F)cc3)o2)cn1]

1-((4-Methoxybenzyl)oxy)-1-methyl-3-(5-((5-(4-(trifluoromethyl)phenyl)oxazol-2-yl)amino)pyridin-2-yl)urea (0.034 g, 0.066 mmol) was dissolved in TFA (1.0 mL) and Et₃SiH (0.1 mL) and the mixture was stirred at rt for 5 h. The volatiles were then removed under reduced pressure and the residue was directly subjected to preparative HPLC using 0.1% TFA in H₂O and 0.1% TFA in ACN as eluents to yield the title product as bright yellow solid (24.14 mg, 93% yield). ¹H NMR (400 MHz, DMSO-d₆) δ 10.70 (s, 1H), 10.13 (s, 1H), 9.20 (s, 1H), 8.57 ((d, J=1.4 Hz, 1H), 8.07 (dd, J=9.1, 2.5 Hz, 1H), 7.85 (d, J=9.2 Hz, 1H), 7.72 (s, 4H), 7.66 (s, 1H), 3.07 (s, 3H). ¹³C NMR (100 MHz, DMSO-d₆) δ 156.80, 156.51, 145.47, 142.89, 131.65, 131.58, 127.06, 126.74, 126.06, 126.02, 125.57, 125.35, 122.87, 113.50, 66.35, 39.52, 37.38. LCMS R_{f}(min)=3.62, MS m/z 393.9 [M+H]⁺. HRMS (ESI) calcd for C₁₇H₁₅F₃N₅O₃⁺ [M+H]⁺394.1122, found 394.1141.

### 36. (R)—N-(2,3-Dihydroxypropyl)-5-((5-(4-(trifluoromethyl)phenyl)oxazol-2-yl)amino)picolinamide (Scheme 34)

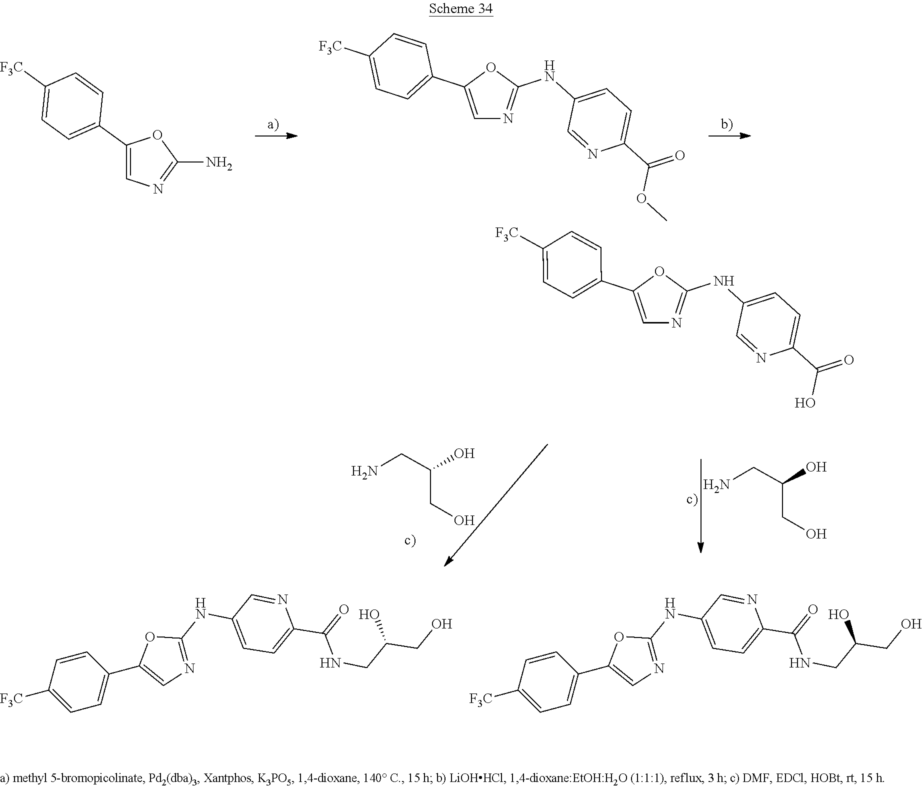

A re-sealable Schlenk tube was charged with Pd₂(dba)₃ (0.02 equiv.), Xantphos (0.06 equiv.), 5-(4-(trifluoromethyl)phenyl)oxazol-2-amine (Intermediate D) (1.2 equiv.), K₃PO₄ (fine powder, 1.4 equiv.), methyl 5-bromopicolinate (0.250 g, 1.152 mmol) and 1,4-dioxane (4 mL). After the mixture was degassed and carefully subjected to three cycles of evacuation and backfilling with N₂, the reaction mixture was heated at 140° C. for 15 h under a N₂ atmosphere. The reaction mixture was cooled, diluted with EtOAc (30 mL) and washed with H₂O (20 mL). The aq. layer was then back-extracted with EtOAc (2×15 mL). The combined organics were concentrated in vacuo to obtain the crude product. This was purified on a SiO₂ column (EtOAc:toluene=1:1) to give methyl 5-((5-(4-(trifluoromethyl)phenyl)oxazol-2-yl)amino)picolinate as a white solid (0.21 g, 49.8% yield). ¹H NMR (400 MHz, DMSO-d₆) δ 11.04 (s, 1H), 8.90 (d, J=2.5 Hz, 1H), 8.44 (s, 1H), 8.36 (dd, J=8.7, 2.6 Hz, 1H), 8.08 (d, J=8.6 Hz, 1H), 8.00 (d, J=8.1 Hz, 2H), 7.78 (d, J=8.4 Hz, 2H), 3.85 (s, 3H). ¹³C NMR (101 MHz, DMSO-d₆) δ 164.2, 155.5, 149.5, 144.3, 138.0, 135.7, 135.68, 134.91, 131.2, 125.7, 125.6, 125.5. LCMS R_{f} (min)=3.907, MS m/z 363.8 [M+H]⁺.

LiOH·H₂O (3 equiv.) in H₂O (2 mL) was added to a solution of methyl 5-((5-(4-(trifluoromethyl)phenyl)oxazol-2-yl)amino)picolinate (0.180 g, 0.495 mmol) in 1,4-dioxane (2 mL) and EtOH (2 mL) and the reaction mixture was refluxed for 3 h. The volatile solvents were removed in vacuo and to the obtained suspension was added brine (2 mL), followed by 6 M HCl (2 mL) dropwise at 0° C. The resulting precipitate was filtered and washed with H₂O (2×2 mL), providing 5-((5-(4-(trifluoromethyl)phenyl)oxazol-2-yl)amino)picolinic acid as a yellow solid (0.240 g, 99.8% yield). ¹H NMR (400 MHz, DMSO-d₆) δ 11.01 (s, 1H), 8.90 (d, J=2.4 Hz, 1H), 8.46 (s, 1H), 8.34 (dd, J=8.6, 2.6 Hz, 1H), 8.07 (d, J=8.6 Hz, 1H), 8.02 (d, J=8.0 Hz, 2H), 7.81 (d, J=8.2 Hz, 2H). LCMS R_{f} (min)=3.578, MS m/z 349.8 [M+H]⁺.

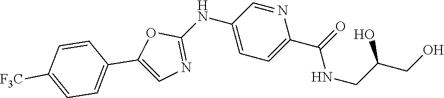 [SMILES: O=C(NC[C@@H](O)CO)c1ccc(Nc2ncc(-c3ccc(C(F)(F)F)cc3)o2)cn1]

5-((5-[4-(Trifluoromethyl)phenyl]-1,3-oxazol-2-yl)amino)picolic acid (0.15 g) was suspended in DMF (3.0 mL), and EDCI (1.3 equiv.) and HOBt (1.4 equiv.) were added. The resulting mixture was stirred at rt for 1 h. (R)-3-Aminopropane-1,2-diol (2.0 equiv.) was then added to the reaction mixture and stirring was continued overnight. The reaction mixture was concentrated under reduced pressure to obtain a gummy solid, which was washed with DCM (2×2 mL) followed by MeOH (2×1 mL) to obtain the title compound as a beige solid (0.095 g, 52% yield). ¹H NMR (400 MHz, DMSO-d₆) δ 11.1 (brs, 1H), 8.80 (d, J=2.3 Hz, 1H), 8.45 (t, J=5.8 Hz, 1H), 8.31 (dd, J=8.6, 2.5 Hz, 1H), 8.05 (d, J=8.6 Hz, 1H), 7.87-7.74 (m, 5H), 4.94 (brs, 1H), 4.65 (brs, 1H), 3.66-3.57 (m, 1H), 3.54-3.45 (m, 1H), (the resonance of 2 protons overlapped with H₂O, and not assigned), 3.26-3.17 (m, 1H). ¹³C NMR (101 MHz, DMSO-d₆) δ 163.7, 156.2, 143.3, 142.7, 138.2, 137.1, 131.4, 127.3, 127.0, 126.0, 125.5, 125.3, 123.5, 123.1, 122.8, 122.6, 70.2, 64.0, 42.4. LCMS R_{f} (min)=3.30. HRMS (ESI) calcd for C₁₉H₁₈F₃N₄O₄⁺ [M+H]⁺ 423.1275, found 423.1289.

### 37. (S)—N-(2,3-Dihydroxypropyl)-5-((5-(4-(trifluoromethyl)phenyl)oxazol-2-yl)amino)picolinamide (Scheme 34)

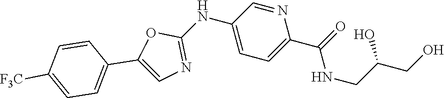 [SMILES: O=C(NC[C@H](O)CO)c1ccc(Nc2ncc(-c3ccc(C(F)(F)F)cc3)o2)cn1]

5-((5-[4-(Trifluoromethyl)phenyl]-1,3-oxazol-2-yl)amino)picolic acid (0.15 g) was suspended in DMF (3.0 mL), and EDCI (1.3 equiv.) and HOBt (1.4 equiv.) were added. The resulting mixture was stirred at rt for 1 h. (S)-3-Aminopropane-1,2-diol (2.0 equiv.) was then added to the reaction mixture and stirring was continued overnight. LCMS indicated the completion of reaction. The reaction mixture was concentrated under reduced pressure to obtain a gummy solid. This was washed with DCM (2×2 mL) followed by MeOH (2×1 mL) to obtain the title compound as a beige solid (0.094 g, 52% yield). ¹H NMR (401 MHz, DMSO-d₆) δ 11.07 (brs, 1H), 8.80 (d, J=2.2 Hz, 1H), 8.45 (t, J=5.7 Hz, 1H), 8.30 (dd, J=8.6, 2.3 Hz, 1H), 8.04 (d, J=8.6 Hz, 1H), 7.94-7.64 (m, 5H), 4.94 (d, J=4.2 Hz, 1H), 4.65 (brs, 1H), 3.61 (m, 1H), 3.56-3.44 (m, 1H), (the resonance of 2 protons overlapped with H₂O, and not assigned), 3.27-3.12 (m, 1H). ¹³C NMR (101 MHz, DMSO-d₆) δ 163.7, 156.2, 143.3, 142.7, 138.2, 137.1, 131.4, 127.3, 126.9, 126.0, 125.5, 125.3, 123.5, 123.0, 122.8, 122.5, 70.3, 64.0, 42.4. LCMS R_{f} (min)=3.306. HRMS (ESI) calcd for C₁₉H₁₈F₃N₄O₄⁺ [M+H]⁺ 423.1275, found 423.1287.

### 38. (R)—N-(2,3-Dihydroxypropyl)-5-((1-(4-(trifluoromethyl)phenyl)-1H-1,2,4-triazol-3-yl)amino)picolinamide (Scheme 35)

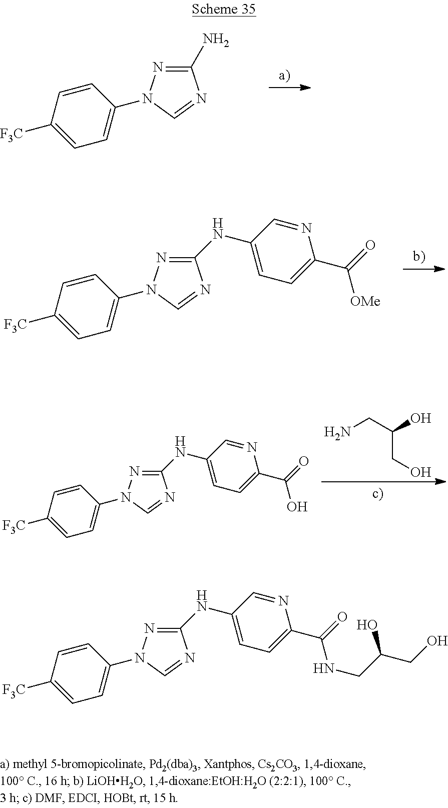 [SMILES: Nc1ncn(-c2ccc(C(F)(F)F)cc2)n1 ; COC(=O)c1ccc(Nc2ncn(-c3ccc(C(F)(F)F)cc3)n2)cn1 ; O=C(O)c1ccc(Nc2ncn(-c3ccc(C(F)(F)F)cc3)n2)cn1 ; NC[C@@H](O)CO ; O=C(NC[C@@H](O)CO)c1ccc(Nc2ncn(-c3ccc(C(F)(F)F)cc3)n2)cn1]

A re-sealable Schlenk tube was charged with Pd₂(dba)₃ (0.05 equiv.), Xantphos (0.1 equiv.), 1-(4-(trifluoromethyl)phenyl)-1H-1,2,4-triazol-3-amine (Intermediate F) (0.3 g), Cs₂CO₃ (1.5 equiv.), methyl 5-bromopicolinate (1.2 equiv.) in 1,4-dioxane (20 mL). The mixture was degassed and carefully subjected to three cycles of evacuation and backfilling with N₂ and heated at 100° C. overnight under N₂ atmosphere. After completion, the mixture was cooled, diluted (100 mL of EtOAc) and washed with H₂O (2×30 mL). The organic layer was separated, dried (MgSO₄), filtered and concentrated under reduced pressure to obtain the crude product. The crude product was washed with Et₂O (3×2.0 mL) to obtain methyl 5-((1-(4-(trifluoromethyl)phenyl)-1H-1,2,4-triazol-3-yl)amino)picolinate as a beige solid (0.262 g, 55.5% yield). ¹H NMR (401 MHz, δ 10.40 (s, 1H), 9.34 (s, 1H), 8.88 (d, J=2.4 Hz, 1H), 8.24 (dd, J=8.7, 2.4 Hz, 1H), 8.12 (d, J=8.4 Hz, 2H), 8.04 (d, J=8.7 Hz, 1H), 7.94 (d, J=8.6 Hz, 2H), 3.84 (s, 3H).

LiOH·H₂O (0.087 g) in H₂O (2.0 mL) was added to a solution of methyl 5-((1-(4-(trifluoromethyl)phenyl)-1H-1,2,4-triazol-3-yl)amino)picolinate in 1,4-dioxane (4 mL) and EtOH (4 mL). The resulting mixture was heated to 100° C. for 3 h. Volatiles were removed in vacuo and the remaining suspension was acidified with 1N HCl (pH-4). The resulting precipitate was filtered and washed with H₂O (3×2 mL), providing 5-((1-(4-(trifluoromethyl)phenyl)-1H-1,2,4-triazol-3-yl)amino)picolinic acid as a yellow solid (0.180 g, 93% yield). ¹H NMR (401 MHz, DMSO-d₆) δ 10.37 (s, 1H), 9.34 (s, 1H), 8.89 (d, J=2.0 Hz, 1H), 8.24 (dd, J=8.6, 2.2 Hz, 1H), 8.12 (d, J=8.3 Hz, 2H), 8.04 (d, J=8.6 Hz, 1H), 7.94 (d, J=8.4 Hz, 2H).

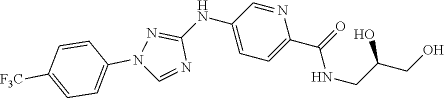 [SMILES: O=C(NC[C@@H](O)CO)c1ccc(Nc2ncn(-c3ccc(C(F)(F)F)cc3)n2)cn1]

EDCI (1.3 equiv.) and HOBt (1.4 equiv.) were added to a suspension of 5-((1-(4-(trifluoromethyl)phenyl)-1H-1,2,4-triazol-3-yl)amino)picolinic acid (0.08 g) in DMF (5.0 mL). The reaction mixture was stirred at rt for 1 h. (R)-3-Aminopropane-1,2-diol (1.5 equiv.) was then added to the reaction mixture and stirring continued overnight. LCMS indicated that the reaction was completed. The reaction mixture was concentrated under reduced pressure to obtain the crude material, which was washed with DCM (2×2 mL) followed by MeOH (2×1 mL) to obtain the title compound as a beige solid (0.055 g, 56.9% yield). ¹H NMR (401 MHz, DMSO-d₆) δ 10.27 (s, 1H), 9.34 (s, 1H), 8.89 (d, J=2.4 Hz, 1H), 8.42 (t, J=5.8 Hz, 1H), 8.22 (dd, J=8.6, 2.5 Hz, 1H), 8.12 (d, J=8.4 Hz, 2H), 8.00 (d, J=8.6 Hz, 1H), 7.95 (d, J=8.6 Hz, 2H), 4.94 (d, J=5.0 Hz, 1H), 4.65 (t, J=5.8 Hz, 1H), 3.64-3.58 (m, 1H), 3.53-3.47 (m, 1H), 3.42-3.27 (m, 2H), 3.25-3.16 (m, 1H). ¹³C NMR (101 MHz, DMSO-d₆) δ 163.9, 160.2, 142.3, 141.3, 140.1, 139.5, 136.7, 127.1, 126.7, 125.4, 122.7, 122.5, 118.6, 70.3, 64.0, 42.3. LCMS R_{f} (min)=3.135. HRMS (ESI) calcd for C₁₈H₁₈F₃N₆O₃⁺ [M+H]⁺ 423.1387, found 423.1395.

### 39. N′,3-Dihydroxy-5-((4-methyl-5-(4-(trifluoromethyl)phenyl)oxazol-2-yl)amino)picolinimidamide (Scheme 36)

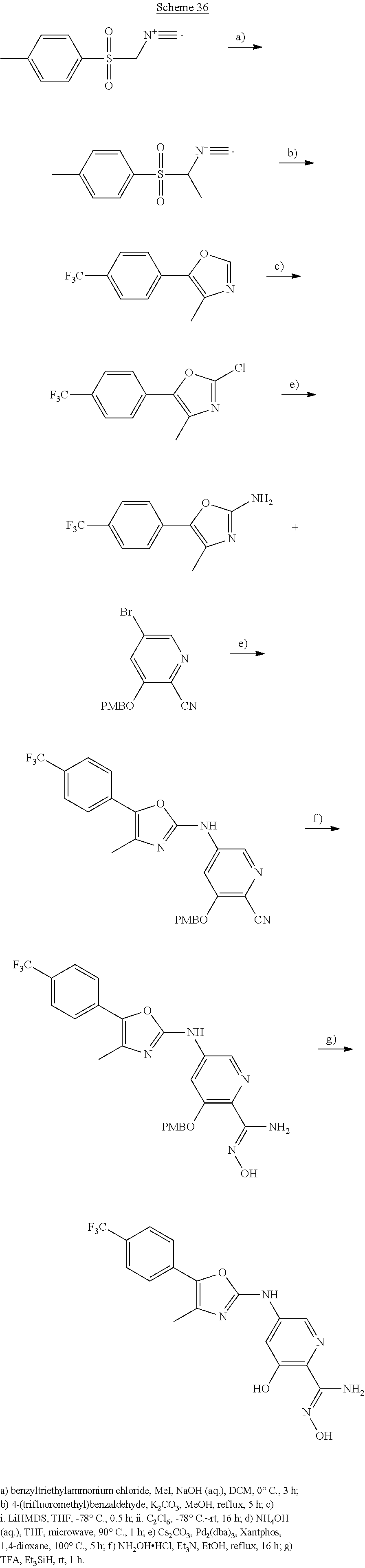

Benzyltriethylammonium chloride (0.12 g, 0.51 mmol), iodomethane (0.72 g, 5.1 mmol) and NaOH (30% aqueous solution, 6 mL) were added to a solution of TosMIC (0.5 g, 2.55 mmol) in DCM (6 mL) at 0° C. The resulting mixture was stirred at 0° C. for 3 h then diluted with DCM (10 mL), washed with water (10 mL×2), dried over MgSO₄, filtered and concentrated to give a brown oil that was subjected to flash chromatography on silica gel (20% EtOAc/hexane), providing 1-[(1-isocyanoethyl)sulfonyl]-4-methylbenzene as a light yellow oil (0.43 g, 80% yield). ¹H NMR (400 MHz, CDCl₃) δ 7.79 (d, J=8.0 Hz, 2H), 7.37 (d, J=8.0 Hz, 2H), 4.63 (q, J=6.8 Hz, 1H), 2.42 (s, 3H), 1.64 (d, J=6.8 Hz, 3H), MS m/z=207.9 [M−H]⁻.

4-(Trifluoromethyl)benzaldehyde (1.25 g, 7.17 mmol) and K₂CO₃ (1.24 g, 8.96 mmol) were added to a solution of 1-((1-isocyanoethyl)sulfonyl)-4-methylbenzene (1.5 g, 7.17 mmol) in MeOH (35 mL) at rt. The resulting mixture was then refluxed for 5 h under nitrogen before being concentrated under reduced pressure. The residue was partitioned between Et₂O and H₂O. The aqueous phase was extracted with Et₂O (20 mL×2), and the combined organic phases were dried over MgSO₄, filtered and concentrated to give a light yellow oil that was subjected to flash chromatography on silica gel (10% EtOAc/hexane), providing 4-methyl-5-[4-(trifluoromethyl)phenyl]oxazole as a white solid (1.4 g, 86% yield). ¹H NMR (400 MHz, CDCl₃) δ 7.87 (s, 1H), 7.68-7.74 (m, 4H), 2.48 (s, 3H), MS m/z=228.0 [M+H]⁺.

LiHMDS (1.0 M in THF, 0.53 mL, 0.53 mmol) was added to a solution of 4-methyl-5-[4-(trifluoromethyl)phenyl]oxazole (0.1 g, 0.44 mmol) in dry THF (3 mL) at −78° C. under nitrogen atmosphere. After a further 0.5 h, a solution of C₂Cl₆ (0.16 g, 0.66 mmol) in THF (1 mL) was treated. The resulting reaction mixture was stirred at −78° C. for another 2 h and allowed to warm to rt over 14 h. The reaction was partitioned with saturated NaHCO₃ (3 mL) and EtOAc (10 mL). The aqueous phase was extracted with EtOAc (8 mL×2). The combined organic phases were dried over Na₂SO₄, filtered, and concentrated to give a brown oil that was subjected to flash chromatography on silica gel (5% EtOAc/hexane), providing 2-chloro-4-methyl-5-[4-(trifluoromethyl)phenyl]oxazole as a white solid (0.11 g, 92% yield). ¹H NMR (400 MHz, CDCl₃) δ 7.65-7.71 (m, 4H), 2.44 (s, 3H), MS m/z=261.9 [M+H]⁺.

Ammonium hydroxide (28-30% aq., 2 mL) was added to a solution of 2-chloro-4-methyl-5-[4-(trifluoromethyl)phenyl]oxazole (0.11 g, 0.43 mmol) in THF (0.5 mL) at rt. The resulting reaction mixture was irradiated under microwave conditions for 1 h at 90° C. The solid was filtered and washed with DCM (2 mL×2), providing 4-methyl-5-[4-(trifluoromethyl)phenyl]oxazol-2-amine as a white solid (0.1 g, 95% yield). ¹H NMR (400 MHz, CDCl₃) δ 7.61 (d, J=8.0 Hz, 2H), 7.54 (d, J=8.0 Hz, 2H), 2.33 (s, 3H). MS m/z 243.0 [M+H]⁺.

An oven-dried RBF was charged with Pd₂(dba)₃ (0.028 g, 0.05 equiv.), Xantphos (0.036 g, 0.1 equiv.), 5-bromo-3-((4-methoxybenzyl)oxy)picolinonitrile (0.217 g, 0.681 mmol, 1.1 equiv.), Cs₂CO₃ (0.303 g, 1.5 equiv.), 4-methyl-5-(4-(trifluoromethyl)phenyl)oxazol-2-amine (0.15 g, 0.619 mmol) and 1,4-dioxane (7 mL). Vacuum was applied briefly to the reaction flask followed by backfilling with N₂ and the procedure was repeated five times. The mixture was then heated to 100° C. and stirred for 5 h. LCMS indicated that the starting materials had been completely consumed. The reaction mixture was then poured into water and extracted with EtOAc (5×). The combined organic solvents were dried over MgSO₄ and evaporated to dryness. The residue was purified on silica gel using mixtures of DCM and EtOAc to provide 3-((4-methoxybenzyl)oxy)-5-((4-methyl-5-(4-(trifluoromethyl)phenyl)oxazol-2-yl)amino)picolinonitrile (0.15 g, 50.4% yield). ¹H NMR (401 MHz, DMSO-d₆) δ 11.34 (s, 1H), 8.33 (d, J=2.0 Hz, 1H), 8.27 (d, J=1.9 Hz, 1H), 7.82 (d, J=8.4 Hz, 2H), 7.73 (d, J=8.3 Hz, 2H), 7.48 (d, J=8.7 Hz, 2H), 6.98 (d, J=8.7 Hz, 2H), 5.26 (s, 2H), 3.76 (s, 3H), 2.45 (s, 3H). ¹³C NMR (101 MHz, DMSO-d₆) δ 159.88, 158.95, 154.46, 140.72, 138.31, 134.92, 133.08, 132.59, 130.35, 127.60, 127.25, 126.94, 126.45, 126.03, 124.75, 123.33, 116.53, 114.48, 113.88, 107.52, 55.59, 14.10. LCMS R_{f} (min)=4.20, MS m/z=480.9 [M+H]⁺.

3-((4-Methoxybenzyl)oxy)-5-((4-methyl-5-(4-(trifluoromethyl)phenyl)oxazol-2-yl)amino)picolinonitrile (0.15 g, 0.312 mmol) was suspended in EtOH (20 mL), then NH₂OH·HCl (0.109 g, 1.561 mmol) was added and approximately half of the EtOH was removed in vacuo. Et₃N (0.213 mL, 1.561 mol) was added and the resulting reaction mixture was stirred at reflux overnight. LCMS indicated that the reaction was complete. The volatile solvents were removed and the resulting solids were suspended in water, collected by filtering then washing well with water. The solids were washed with Et₂O then dried under high vacuum to provide N-hydroxy-3-((4-methoxybenzyl)oxy)-5-((4-methyl-5-(4-(trifluoromethyl)phenyl)oxazol-2-yl)amino)picolinimidamide (0.081 g, 50.5% yield). ¹H NMR (401 MHz, DMSO-d₆) δ 10.77 (s, 1H), 9.62 (s, 1H), 8.34 (d, J=2.0 Hz, 1H), 8.01 (d, J=2.0 Hz, 1H), 7.81 (d, J=8.4 Hz, 2H), 7.71 (d, J=8.3 Hz, 2H), 7.46 (d, J=8.7 Hz, 2H), 6.94 (d, J=8.7 Hz, 2H), 5.59 (s, 2H), 5.13 (s, 2H), 3.75 (d, J=5.6 Hz, 3H), 2.08 (s, 3H). ¹³C NMR (101 MHz, DMSO-d₆) δ 159.39, 155.36, 153.54, 150.27, 137.64, 137.18, 135.11, 134.15, 132.89, 129.69, 129.57, 128.87, 126.88, 126.57, 126.47, 126.09, 124.46, 123.39, 114.26, 109.64, 55.54, 14.18. LCMS R_{f} (min)=3.236. HRMS (ESI) calcd for C₂₅H₂₃F₃N₅O₄⁺ [M+H]⁺ 514.1697, found 514.1687.

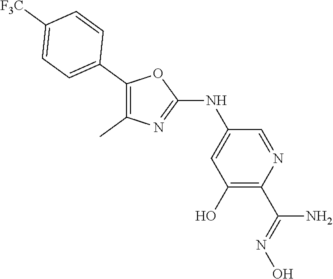 [SMILES: Cc1nc(Nc2cnc(/C(N)=N/O)c(O)c2)oc1-c1ccc(C(F)(F)F)cc1]

N′-hydroxy-3-((4-methoxybenzyl)oxy)-5-((4-methyl-5-(4-(trifluoromethyl)phenyl)oxazol-2-yl)amino)picolinimidamide (71.9 mg, 0.140 mmol) was stirred in a mixture of TFA (1.4 mL) and Et₃SiH (0.07 mL) at rt for 1 h. The reaction mixture was evaporated to dryness. The residue was purified on preparative HPLC to provide the title compound (40.9 mg, 74.26%) as a pale yellow solid. 1H NMR (401 MHz, DMSO-d₆) δ 11.00 (s, 1H), 10.79-10.21 (brs, 1H), 8.29 (d, J=2.2 Hz, 1H), 7.93 (d, J=2.0 Hz, 1H), 7.82 (d, J=8.4 Hz, 2H), 7.73 (d, J=8.3 Hz, 2H), 7.70-7.76 (brs, 1H), 2.41 (s, 3H). ¹³C NMR (101 MHz, DMSO-d₆) δ 154.8 (C), 154.6 (C), 137.5 (C), 134.6 (C), 132.3 (C), 129.9 (CH), 126.6 (C), 126.3 (C), 126.0 (CH), 125.6 (C), 124.2 (CH), 122.9 (C), 110.1 (CH), 13.7 (C), 13.6 (CH₃). LCMS: R_{f} (min)=3.688, MS m/z=393.9 [M+H]⁺. HRMS (ESI) calcd for C₁₇H₁₅F₃N₅O₃⁺ [M+H]⁺ 394.1122, found 394.1140.

### 40. N′,3-Dihydroxy-5-((5-(5-(trifluoromethyl)pyridin-2-yl)oxazol-2-yl)amino)picolinimidamide hydrochloride (Scheme 37)

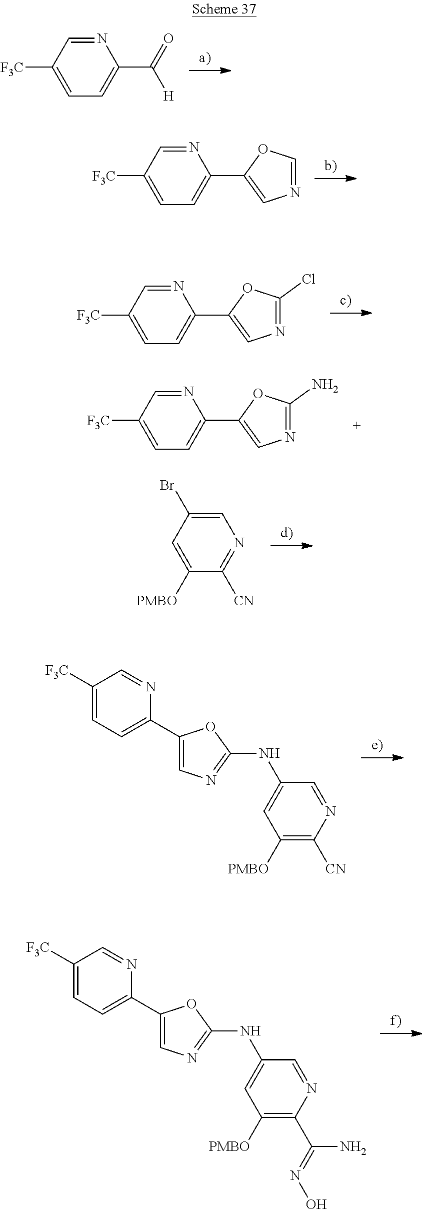

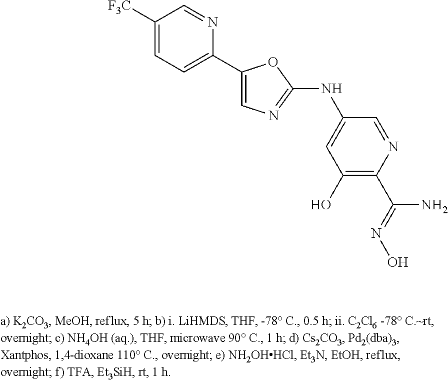 [SMILES: N/C(=N\O)c1ncc(Nc2ncc(-c3ccc(C(F)(F)F)cn3)o2)cc1O]

i-PrMgCl (2 M in THF, 10.0 mL, 20.0 mmol) was added to a solution of 2-bromo-5-(trifluoromethyl)pyridine (4.0 g, 17.7 mmol) in DCM (550 mL) at 0° C. over 5 minutes. After stirring at 0-6° C. for 40 minutes, the mixture was cooled to −20° C. and DMF (2.8 mL, 35.4 mmol) was added in one portion. The mixture was warmed to rt over 2 h, then quenched by addition of sat. NaHCO₃ aqueous solution (15 mL). After stirring for 10 minutes, the suspension was filtered through a celite pad. The filtrate layers were separated. The celite pad was further washed with 20 mL DCM and the organic liquid was then used to re-extract the aqueous layer. The combined organic phases were dried over MgSO₄, filtered, and concentrated under reduced pressure. The resultant crude (˜3.6 g) was used in the next step without further characterisations.

5-(Trifluoromethyl)picolinaldehyde (0.65 g, 3.71 mmol) and K₂CO₃ (0.62 g, 4.45 mmol) were added to a solution of 1-((isocyanomethyl)sulfonyl)-4-methylbenzene (0.80 g, 4.08 mmol) in MeOH (10 mL) at rt. The resulting mixture was refluxed for 5 h under nitrogen before being concentrated under reduced pressure. The residue was partitioned between Et₂O and H₂O. The aqueous phase was extracted with Et₂O (20 mL×2), and the combined organic phases were dried over MgSO₄, filtered and concentrated to give a light yellow oil that was subjected to flash chromatography on silica gel (3% to 20%, EtOAc/hexane). Collection of the appropriate fractions provided 5-(5-(trifluoromethyl)pyridin-2-yl)oxazole as a white solid. (0.65 g, 82% yield). ¹H NMR (400 MHz, CDCl₃) δ 8.87 (m, 1H), 8.02 (s, 1H), 8.00 (m, 1H), 7.82 (s, 1H), 7.77 (d, J=8.3 Hz, 1H). MS m/z=215.0 [M+H]⁺.

### Intermediate J—2-chloro-5-(5-(trifluoromethyl)pyridin-2-yl)oxazole

LiHMDS (1.0 M in THF, 5.88 mL, 5.88 mmol) was added to a solution of 5-(5-(trifluoromethyl)pyridin-2-yl)oxazole (1.05 g, 4.90 mmol) in dry THF (10 mL) at −78° C. under nitrogen atmosphere. After a further 0.5 h, a solution of C₂Cl₆ (1.74 g, 7.35 mmol) in THF (5 mL) was treated. The resulting reaction mixture was stirred at −78° C. for another 2 h and allowed to warm to rt over 14 h. The reaction was quenched with saturated NaHCO₃ (10 mL). The aqueous phase was extracted with EtOAc (30 mL×2). The combined organic phases were dried over Na₂SO₄, filtered, and concentrated to give a brown oil that was subjected to flash chromatography on silica gel (2%˜10%, EtOAc/hexane). Collection of the appropriate fractions provided 2-chloro-5-(5-(trifluoromethyl)pyridin-2-yl)oxazole (Intermediate J) as a white solid (1.04 g, 85% yield). ¹H NMR (400 MHz, CDCl₃) δ 8.86 (m, 1H), 8.00 (m, 1H), 7.75 (s, 1H), 7.71 (d, J=8.0 Hz, 1H). MS m/z=248.9 [M+H]⁺.

### Intermediate K—5-(5-(trifluoromethyl)pyridin-2-yl)oxazol-2-amine

Ammonium hydroxide (28-30% aqueous solution, 10 mL) was added to a solution of 2-chloro-5-(5-(trifluoromethyl)pyridin-2-yl)oxazole (0.60 g, 2.41 mmol) in THF (1.5 mL) at rt. The resulting reaction mixture was irradiated under microwave at 90° C. for 1 h. The solid was filtered and washed with DCM (5 mL×2), providing 5-(5-(trifluoromethyl)pyridin-2-yl)oxazol-2-amine as a white solid (0.52 g, 95% yield). ¹H NMR (400 MHz, DMSO-d₆) δ 8.81 (t, J=1.5 Hz, 1H), 8.13 (dd, J=8.4, 1.5 Hz, 1H), 7.60 (s, 1H), 7.57 (d, J=8.4 Hz, 1H), 7.33 (s, 2H). MS m/z=230.0 [M+H]⁺.

An oven-dried RBF was charged with Pd₂(dba)₃ (0.030 g, 0.05 equiv.), Xantphos (0.038 g, 0.1 equiv.), 5-bromo-3-((4-methoxybenzyl)oxy)picolinonitrile (0.209 g, 0.681 mmol, 1.0 equiv.), Cs₂CO₃ (0.320 g, 1.5 equiv.), 5-(5-(trifluoromethyl)pyridin-2-yl)oxazol-2-amine (0.15 g, 0.654 mmol) and 1,4-dioxane (7 mL). Vacuum was applied briefly to the reaction flask followed by backfilling with N₂ and the procedure was repeated five times. The mixture was then heated to 110° C. and stirred for 18 h. LCMS indicated that the starting materials had been completely consumed. The reaction mixture was poured into water and extracted with EtOAc (5×). The combined organic solvents were dried over MgSO₄ and evaporated to dryness. The residue was purified on silica gel using mixtures of DCM and EtOAc to provide 3-((4-methoxybenzyl)oxy)-5-((5-(5-(trifluoromethyl)pyridin-2-yl)oxazol-2-yl)amino)picolinonitrile (0.215 g, 70.3% yield). ¹H NMR (401 MHz, DMSO-d₆) δ 11.65 (s, 1H), 8.95 (s, 1H), 8.36 (d, J=1.9 Hz, 1H), 8.28 (m, 2H), 7.98 (s, 1H), 7.81 (d, J=8.4 Hz, 1H), 7.52-7.43 (d, J=8.7 Hz, 2H), 6.99 (d, J=8.7 Hz, 2H), 5.22 (s, 2H), 3.79 (s, 3H). ¹³C NMR (101 MHz, DMSO-d₆) δ 159.91, 158.99, 156.98, 150.22, 144.34, 140.59, 135.26, 133.21, 130.48, 129.28, 127.54, 118.43, 116.45, 114.48, 114.27, 107.77, 70.71, 55.61. LCMS R_{f} (min)=3.626, MS m/z=467.9 [M+H]⁺.

3-((4-Methoxybenzyl)oxy)-5-((5-(5-(trifluoromethyl)pyridin-2-yl)oxazol-2-yl)amino)picolinonitrile (0.20 g, 0.427 mmol) was suspended in EtOH (30 mL), then NH₂OH·HCl (0.238 g, 3.423 mmol) was added and approximately half of the EtOH was removed in vacuo. Et₃N (0.467 mL, 3.423 mol) was added and the resulting reaction mixture was stirred at reflux overnight. LCMS indicated that the reaction was complete. The volatile solvents were removed and the resulting solids were suspended in water, collected by filtering and washing well with water. The solids were washed with Et₂O and dried under high vacuum to provide N′-hydroxy-3-((4-methoxybenzyl)oxy)-5-((5-(5-(trifluoromethyl)pyridin-2-yl)oxazol-2-yl)amino)picolinimidamide (0.183 g, 85.75% yield). ¹H NMR (401 MHz, DMSO-d₆) δ 11.09 (s, 1H), 9.61 (s, 1H), 8.93 (s, 1H), 8.36 (s, 1H), 8.26 (d, J=7.7 Hz, 1H), 8.01 (s, 1H), 7.92 (s, 1H), 7.77 (d, J=8.2 Hz, 1H), 7.47 (d, J=7.7 Hz, 2H), 6.94 (d, J=7.8 Hz, 2H), 5.60 (s, 2H), 5.12 (s, 2H), 3.75 (s, 3H). ¹³C NMR (101 MHz, DMSO-d₆) δ 159.41, 157.87, 153.61, 150.43, 150.23, 147.04, 143.79, 136.96, 135.21, 134.62, 129.87, 129.72, 129.57, 128.79, 125.62, 123.17, 122.85, 118.08, 114.25, 109.80, 70.07, 55.54. LCMS R_{f} (min)=3.082, MS m/z=500.9 [M+H]⁺.

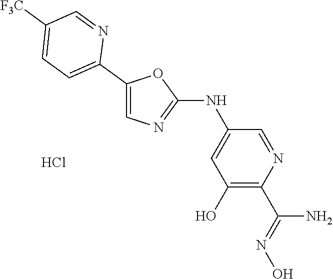 [SMILES: N/C(=N\O)c1ncc(Nc2ncc(-c3ccc(C(F)(F)F)cn3)o2)cc1O ; Cl]

N′-Hydroxy-3-((4-methoxybenzyl)oxy)-5-((5-(5-F₃C (trifluoromethyl)pyridin-2-yl)oxazol-2-yl)amino)-picolinimidamide (170 mg, 0.339 mmol) was stirred in a mixture of TFA (3 mL) and Et₃SiH (0.15 mL) at rt for 4 h. The reaction mixture was evaporated to dryness. The residue was purified on preparative HPLC, then freeze-dried from 4N HCl dioxane solution to provide N′,3-dihydroxy-5-((5-(5-(trifluoromethyl)pyridin-2-yl)oxazol-2-yl)amino)picolinimidamide hydrogen chloride (104 mg, 66.43% yield, corrected for presence of HCL salt and 0.5 molar equivalent of dioxane). ¹H NMR (401 MHz, DMSO-d₆) δ 12.30-12.03 (br, 1H), 11.54 (s, 1H), 10.90 (brs, 1H), 8.94 (d, J=0.9 Hz, 1H), 8.50 (br, 1H), 8.37 (d, J=2.1 Hz, 1H), 8.28 (dd, J=8.5, 2.0 Hz, 1H), 8.12 (s, 1H), 7.95 (s, 1H), 7.85 (d, J=8.4 Hz, 1H). ¹³C NMR (101 MHz, DMSO-d₆) δ 157.32, 155.51, 150.30, 147.06, 144.20, 140.12, 135.26, 131.19, 129.37, 125.58, 123.39, 118.32, 110.89. LCMS R_{f} (min)=3.682, MS m/z=380.9 [M+H]⁺. HRMS (ESI) calcd for C₁₅H₁₂F₃N₆O₃⁺ [M+H]⁺ 381.0917, found 381.0928.

### 41. (R)—N-(2,3-Dihydroxypropyl)-6-((5-(4-(trifluoromethyl)phenyl)oxazol-2-yl)amino)-pyridazine-3-carboxamide (Scheme 38)

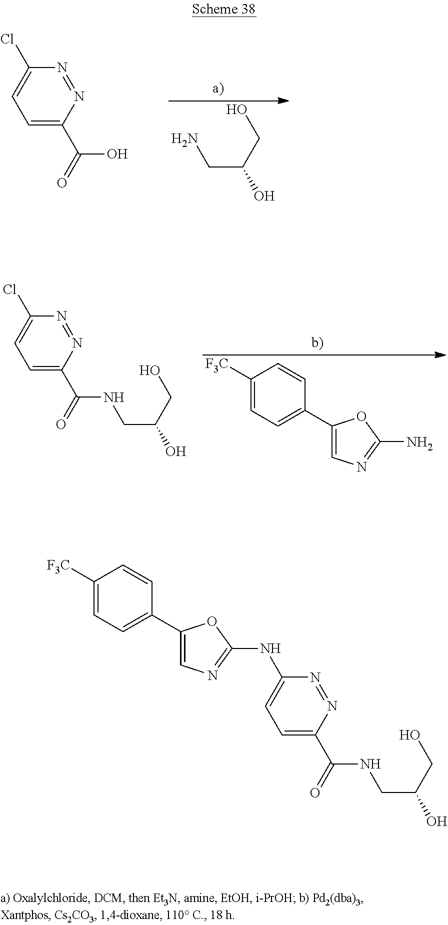 [SMILES: O=C(O)c1ccc(Cl)nn1 ; NC[C@@H](O)CO ; O=C(NC[C@@H](O)CO)c1ccc(Cl)nn1 ; Nc1ncc(-c2ccc(C(F)(F)F)cc2)o1 ; O=C(NC[C@@H](O)CO)c1ccc(Nc2ncc(-c3ccc(C(F)(F)F)cc3)o2)nn1]

6-Chloropyridazine-3-carboxylic acid (0.57 g, 3.595 mmol) was dissolved in DCM (18 mL) with a catalytic amount of DMF. Oxalylchloride (0.617 mL, 4.19 mmol, 2 equivalents) was added dropwise, and the resulting solution was stirred at rt on. The volatile solvents were removed in vacuo, and the residue was dried under high vacuum. (R)-3-Aminopropane-1,2-diol (0.982 g, 10.78 mmol, 3 equiv.), and Et₃N (1.45 mL, 10.78 mmol, 3 equiv.) were dissolved in a mixture of ⁱPrOH (5 mL) and EtOH (5 mL). The amine solution was added slowly to the solid residue of the crude acid chloride, and the resultant reaction mixture was stirred at rt on. All volatile solvents were removed in vacuo and the residue freeze-dried from water. The crude product was dissolved in MeOH, and treated with IR120 ion exchange resins to remove excess amines and Et₃N. The resins were washed well with MeOH, and the combined MeOH washing solutions were evaporated to dryness. The solid residue was purified on silica gel using DCM and MeOH mixtures as eluents to provide (R)-6-chloro-N-(2,3-dihydroxypropyl)pyridazine-3-carboxamide (0.477 g, 57.27%). ¹H NMR (401 MHz, DMSO-d₆) δ 8.98 (s, 1H), 8.24 (d, J=8.7 Hz, 1H), 8.10 (d, J=8.7 Hz, 1H), 4.92 (br, 1H), 4.65 (br, 1H), 3.68 (br, 1H), 3.55-3.46 (m, 1H), 3.29 (m, 3H). ¹³C NMR (101 MHz, DMSO-d₆) δ 162.12, 158.66, 152.91, 130.75, 129.27, 70.38, 64.43, 43.28. LCMS R_{f} (min)=1.393, MS m/z=231.9 [M+H]⁺.

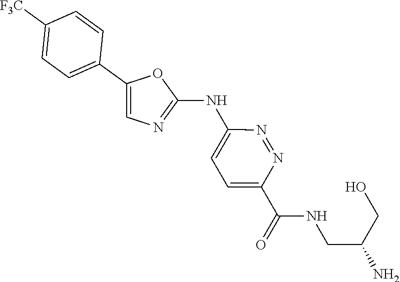 [SMILES: N[C@@H](CO)CNC(=O)c1ccc(Nc2ncc(-c3ccc(C(F)(F)F)cc3)o2)nn1]

An oven-dried RBF was charged with Pd₂(dba)₃ (0.020 g, 0.05 equiv.), Xantphos (0.025 g, 0.1 equiv.), (R)-6-chloro-N-(2,3-dihydroxypropyl)pyridazine-3-carboxamide (0.101 g, 0.438 mmol, 1.0 equiv.), Cs₂CO₃ (0.214 g, 1.5 equiv.), 5-(4-(trifluoromethyl)-phenyl)oxazol-2-amine (0.1 g, 0.438 mmol) and 1,4-dioxane (8 mL). Vacuum was applied briefly to the reaction flask followed by backfilling with N₂ and the procedure was repeated five times. The mixture was then heated to 110° C. and stirred for 18 h. LCMS indicated that the starting materials had been completely consumed. The volatile solvents were removed in vacuo, and the residue triturated with water. The solid precipitates were collected via filtering, washed with 5% potassium xanthate (2×) aqueous solutions, 10% citric acid aqueous solutions, water. The residual solids was purified on preparative HPLC to provide (R)—N-(2,3-dihydroxypropyl)-6-((5-(4-(trifluoromethyl)-phenyl)oxazol-2-yl)amino)pyridazine-3-carboxamide (0.072 g, 38.8% yield). ¹H NMR (401 MHz, DMSO-d₆) δ 12.13 (s, 1H), 8.74 (s, 1H), 8.46 (s, 1H), 8.20 (d, J=9.2 Hz, 1H), 7.82 (s, 4H), 7.815 (s, 1H), 4.94 (d, J=5.0 Hz, 1H), 4.65 (t, J=5.6 Hz, 1H), 3.66 (m, 1H), 3.52 (m, 1H), 3.38 (m, 2H), 3.27 (m, 1H). ¹³C NMR (101 MHz, DMSO-d₆) δ 162.90, 144.38, 131.79, 128.16, 128.01, 127.69, 126.58, 126.54, 126.00, 123.76, 123.30, 70.56, 64.44, 43.02. LCMS R_{f}(min)=4.032. HRMS (ESI) calcd for C₁₈H₁₇F₃N₅O₄⁺ [M+H]⁺ 424.1227, found 424.1225.

### 42. N′,4-Dihydroxy-6-((5-(4-(trifluoromethyl)phenyl)oxazol-2-yl)amino)pyridazine-3-carboximidamide (Scheme 39)

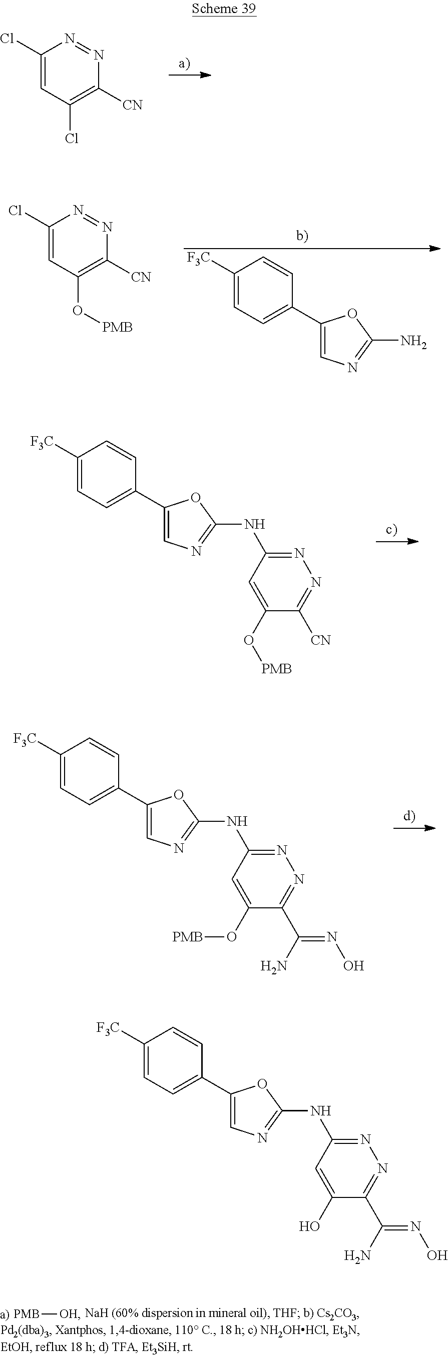

p-Methoxybenzyl alcohol (0.121 g, 0.879 mmol, 1.0 equiv.) was dissolved in dry THF (5 mL), and NaH (60%, 0.021 g, 0.879 mmol, 1.0 equiv.) added under N₂. After 10 minutes of stirring, the solution was transferred to a flask containing 4,6-dichloropyridazine-3-carbonitrile (0.153 g, 0.879 mmol) in dry THF (5 mL). The resultant reaction mixture was stirred at rt for 1 h. LCMS indicated that all starting material had been consumed. The reaction mixture was poured into sat. aq. NaHCO₃ solution and the product extracted with DCM (5×). The combined organic solutions were dried over MgSO₄, and evaporated to dryness. The residue was purified on silica gel using petroleum spirit and EtOAc mixtures as eluents to provide 6-chloro-4-((4-methoxybenzyl)oxy)pyridazine-3-carbonitrile (0.127 g, 52.38%). ¹H NMR (401 MHz, CDCl₃) δ 7.35 (d, J=8.6 Hz, 2H), 7.16 (s, 1H), 6.95 (d, J=8.6 Hz, 2H), 5.24 (s, 2H), 3.82 (s, 3H). ¹³C NMR (101 MHz, CDCl₃) δ 160.55, 159.42, 158.49, 132.41, 129.59, 124.34, 114.64, 112.29, 110.58, 72.15, 55.41. LCMS R_{f} (min)=3.298, MS m/z=297.9 [M+Na]⁺.

An oven-dried RBF was charged with Pd₂(dba)₃ (0.021 g, 0.05 equiv.), Xantphos (0.026 g, 0.1 equiv.), 5-(4-(trifluoromethyl)phenyl)oxazol-2-amine (0.119 g, 0.544 mmol, 1.2 equiv.), Cs₂CO₃ (0.221 g, 1.5 equiv.), 6-chloro-4-((4-methoxybenzyl)oxy)pyridazine-3-carbonitrile (0.125 g, 0.453 mmol) and 1,4-dioxane (8 mL). Vacuum was applied briefly to the reaction flask followed by backfilling with N₂ and the procedure was repeated five times. The mixture was then heated to 110° C. and stirred for 18 h. LCMS indicated that the starting materials had been completely consumed. The reaction mixture was then poured into water and extracted with EtOAc (5×). The combined organic solvents were dried over MgSO₄ and evaporated to dryness. The residue was purified on silica gel using mixtures of DCM, EtOAc, and MeOH to provide 4-((4-methoxybenzyl)oxy)-6-((5-(4-(trifluoromethyl)phenyl)oxazol-2-yl)amino)-pyridazine-3-carbonitrile (0.046 g, 21.7% yield). The purity was ˜80%, and the product was used in the next step without further purification. LCMS R_{f} (min)=4.47, MS m/z=467.9 [M+H]⁺.

4-((4-Methoxybenzyl)oxy)-6-((5-(4-(trifluoromethyl)phenyl)oxazol-2-yl)amino)pyridazine-3-carbonitrile (0.046 g, 0.098 mmol) was suspended in EtOH (10 mL), then NH₂OH·HCl (0.055 g, 0.787 mmol) was added and approximately half of the EtOH was removed in vacuo. Et₃N (0.107 mL, 0.787 mmol) was added and the resulting reaction mixture was stirred at reflux overnight. LCMS indicated that the reaction was complete. The volatile solvents were removed and the resulting solids were suspended in water, collected by filtering then washing well with water. The solids were washed with Et₂O then dried under high vacuum to provide N′-hydroxy-4-((4-methoxybenzyl)oxy)-6-((5-(4-(trifluoromethyl)phenyl)oxazol-2-yl)amino)pyridazine-3-carboximidamide (0.037 g, 75.12% yield). The product was used in the next step without further purification LCMS R_{f} (min)=3.717, MS m/z=500.9 [M+H]⁺.

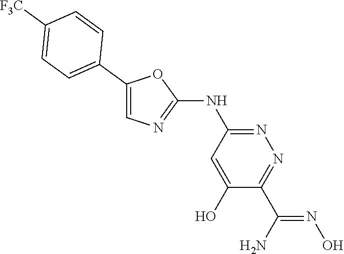 [SMILES: N/C(=N\O)c1nnc(Nc2ncc(-c3ccc(C(F)(F)F)cc3)o2)cc1O]

N′-Hydroxy-4-((4-methoxybenzyl)oxy)-6-((5-(4-(trifluoromethyl)phenyl)oxazol-2-yl)amino)pyridazine-3-carboximidamide (37 mg, 0.069 mmol) was stirred in a mixture of TFA (2 mL) and Et₃SiH (0.1 mL) at rt for 4 h. The reaction mixture was evaporated to dryness. The residue was purified on preparative HPLC to provide N′,4-dihydroxy-6-((5-(4-(trifluoromethyl)phenyl)oxazol-2-yl)amino)pyridazine-3-carboximidamide (5.3 mg, 19.96% yield). ¹H NMR (401 MHz, DMSO-d₆) δ 10.79 (s, 1H), 7.87 (s, 1H), 7.81 (m, 4H), 7.59 (br, 3H). LCMS R_{f} (min)=3.615, MS m/z=380.9 [M+H]⁺. HRMS (ESI) calcd for C₁₅H₁₂F₃N₆O₃⁺ [M+H]⁺ 381.0917, found 381.0912.

### 43. 5-((5-(3-Fluoro-4-(trifluoromethyl)phenyl)oxazol-2-yl)amino)-N′,3-dihydroxypicolinimidamide (Scheme 40)

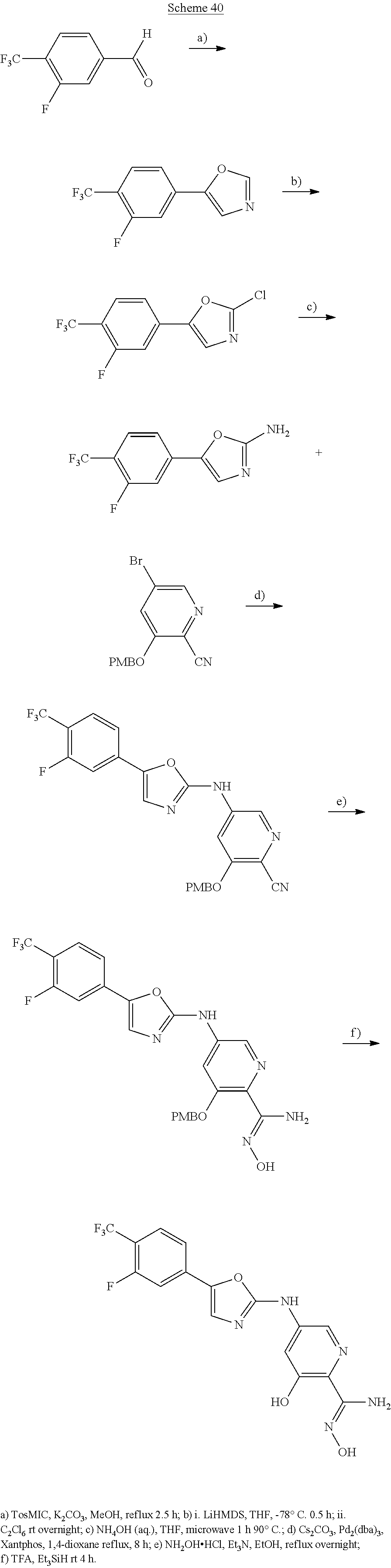

3-Fluoro-4-(trifluoromethyl)benzaldehyde (2.5 g, 13.01 mmol), p-toluenesulfonylmethyl isocyanide (2.723 g, 13.01 mmol), K₂CO₃ (2.158 g, 15.61 mmol) were added into a dry RBF and refluxed with MeOH (7 mL) for 2.5 h. After this period of time the solvent was evaporated and aq. NaHCO₃ solution (20 mL) was added. The suspension was extracted with DCM (3×25 mL). The combined organic fractions were washed with brine, dried over MgSO₄, filtered and concentrated under reduced pressure. The residue was chromatographed on silica gel eluting with 50% EtOAc in hexane to afford 5-(3-fluoro-4-(trifluoromethyl)phenyl)oxazole as white solid (2.808 g, 94%). ¹H NMR (400 MHz, DMSO-d₆) δ 8.54 (s, 1H), 7.93 (s, 1H), 7.78 (dd, J=10.0, 5.4 Hz, 2H), 7.65 (d, J=8.3 Hz, 1H). ¹³C NMR (101 MHz, DMSO-d₆) δ 160.51, 157.99, 153.09, 148.09, 133.70, 128.21, 125.42, 120.01, 112.33, 112.10. LCMS R_{f} (min)=3.461, MS m/z=232.0 [M+H]⁺.

LiHMDS (1.00 M in THF, 14.75 mL, 14.02 mmol) was added to a solution of 5-(3-fluoro-4-(trifluoromethyl)phenyl)oxazole (2.70 g, 11.68 mmol) in THF (90 mL) at −78° C. The mixture was stirred at −78° C. for 1 h. After this period of time a solution of C₂Cl₆ (4.15 g, 17.52 mmol) in THF (6 mL) was added at −78° C., and the resultant mixture was allowed to stir at −78° C. for 2 h. Then the mixture was allowed to warm to rt and stirred further for 14 h. The reaction was quenched H₂O (15 mL), and diluted with EtOAc (20 mL). The organic layer was extracted with EtOAc (3×20 mL). The combined organics were washed with brine (20 mL), dried over MgSO₄, filtered and evaporated to dryness. The resultant white crystalline solid was used in next step without further purification (2.825 g, 94%). ¹H NMR (400 MHz, CDCl₃) δ 7.65 (t, J=7.7 Hz, 1H), 7.49-7.37 (m, 3H). ¹³C NMR (101 MHz, DMSO-d₆) δ 160.53, 158.02, 153.09, 148.12, 133.70, 128.21, 128.15, 125.42, 120.05, 112.10. LCMS R_{f} (min)=3.745, MS m/z=265.8 [M+H]⁺.

NH₄OH_{(aq)} solution (28-30% NH₃ basis, 35 mL) was added in to a microwave vial containing 2-chloro-5-(3-fluoro-4-(trifluoromethyl)phenyl)oxazole (0.577 g, 2.173 mmol) in THF (4 mL) and the mixture was subjected to microwave irradiation for 1 h at 90° C. The resulting suspension was filtered, washed with Et₂O and dried under vacuum to yield 5-(3-fluoro-4-(trifluoromethyl)phenyl)oxazol-2-amine as yellow solid (0.494 g, 92%). ¹H NMR (400 MHz, MeOH-d₄) δ 7.61 (t, J=7.8 Hz, 1H), 7.41 (t, J=10.5 Hz, 2H), 7.31 (s, 1H). ¹³C NMR (101 MHz, DMSO-d₆) δ 162.21, 158.42, 140.69, 134.82, 134.72, 128.21, 127.14, 117.59, 109.58, 109.35. ¹³C NMR (101 MHz, DMSO-d₆) δ 162.66, 161.14, 158.85, 141.14, 135.16, 128.65, 127.58, 121.86, 118.02, 110.02. LCMS R_{f} (min)=3.273, MS m/z=246.9 [M+H]⁺.

5-(3-Fluoro-4-(trifluoromethyl)phenyl)oxazol-2-amine (0.300 g, 1.219 mmol), 5-bromo-3-((4-methoxybenzyl)oxy)picolinonitrile (0.466 g, 1.462 mmol), Cs₂CO₃ (0.596 g, 1.828 mmol), Pd₂(dba)₃ (0.056 g, 0.0609 mmol), 4,5-bis(diphenylphosphino)-9,9-dimethylxanthene (0.0705 g, 0.122 mmol) and anhydrous 1,4-dioxane (16 mL) were added into a dry RBF. The mixture was degassed, refilled with N_{2(g)}(×5) and was refluxed for 8 h under N_{2(g)}. The reaction mixture was quenched with aq. NaHCO₃ solution (10 mL) and diluted with EtOAc (15 mL). The organic layer was extracted with EtOAc (4×15 mL). The combined organic fractions were dried over MgSO₄, filtered and evaporated to dryness. The residue (0.426 g) was subjected to next step without further purification. LCMS R_{f} (min)=3.732, MS m/z=484.9 [M+H]⁺.

5-((5-(3-Fluoro-4-(trifluoromethyl)phenyl)oxazol-2-yl)amino)-3-((4-methoxybenzyl)oxy)picolinonitrile (0.426 g) was suspended in EtOH (12 mL) and NH₂OH·HCl (0.148 g, 2.132 mmol) was added to the mixture while stirring at rt. Then, EtOH volume was halved (˜6 mL) under vacuum, Et₃N (0.30 mL, 2.132 mmol) was added and the mixture was allowed to stir at reflux for 24 h. After this period of time the volatiles were evaporated to dryness. The residue was washed with H₂O (15 mL) and Et₂O (20 mL) to yield 5-((5-(3-fluoro-4-(trifluoromethyl)phenyl)oxazol-2-yl)amino)-N′-hydroxy-3-((4-methoxybenzyl)oxy)picolinimidamide as bright orange solid (0.325 g, 76%). ¹H NMR (400 MHz, DMSO-d₆) δ 10.61 (s, 1H), 8.11-8.06 (m, 1H), 7.83 (q, J=5.3, 3.4 Hz, 3H), 7.67 (d, J=11.8 Hz, 1H), 7.54 (d, J=8.0 Hz, 1H), 7.48 (d, J=8.5 Hz, 2H), 7.35 (s, 1H), 6.97 (d, J=8.5 Hz, 2H), 5.14 (s, 2H), 3.76 (s, 3H). ¹³C NMR (101 MHz, DMSO-d₆) δ 159.79, 157.89, 155.06, 142.14, 141.84, 140.88, 140.59, 138.85, 138.79, 138.68, 133.23, 131.03, 130.47, 130.00, 128.11, 127.65, 125.91, 118.71, 114.38, 114.30, 110.33, 70.35, 55.58. LCMS R_{f} (min)=3.350, MS m/z=517.9 [M+H]⁺.

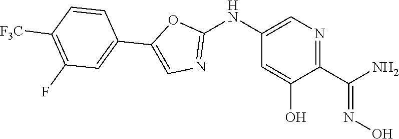 [SMILES: N/C(=N\O)c1ncc(Nc2ncc(-c3ccc(C(F)(F)F)c(F)c3)o2)cc1O]

TFA (2.0 mL) and Et₃SiH (0.2 mL) were added into a RBF with 5-((5-(3-fluoro-4-(trifluoromethyl)phenyl)oxazol-2-yl)amino)-N′-hydroxy-3-((4-methoxybenzyl)oxy)picolinimidamide (0.1124 g, 0.217 mmol) and the resulting mixture was stirred at RT for 4 h. The volatiles were removed under reduced pressure and the residue was directly subjected to preparative HPLC to provide 5-((5-(3-fluoro-4-(trifluoromethyl)phenyl)oxazol-2-yl)amino)-N′,3-dihydroxypicolinimidamide as orange solid (0.069 g, 80%, 100% purity). ¹H NMR (400 MHz, DMSO-d₆) δ 11.15 (s, 1H), 10.5 (brs, 1H), 8.30 (d, J=2.1 Hz, 1H), 7.96-7.88 (m, 3H), 7.73 (d, J=12.0 Hz, 1H), 7.59 (d, J=8.3 Hz, 1H). ¹³C NMR (101 MHz, DMSO-d₆) δ 158.25, 158.10, 156.55, 154.67, 142.26, 134.11, 129.86, 128.49, 128.47, 126.98, 123.99, 118.58, 118.55, 110.72, 110.38. LCMS R_{f}(min)=3.341, MS m/z=397.9 [M+H]⁺. HRMS (ESI) calcd for C₁₆H₁₂F₄N₅O₃⁺ [M+H]⁺ 398.0871, found 398.0877.

### 44. (R)—N-(2,3-Dihydroxypropyl)-5-((5-(3-fluoro-4-(trifluoromethyl)phenyl)oxazol-2-yl)amino)picolinamide (Scheme 41)

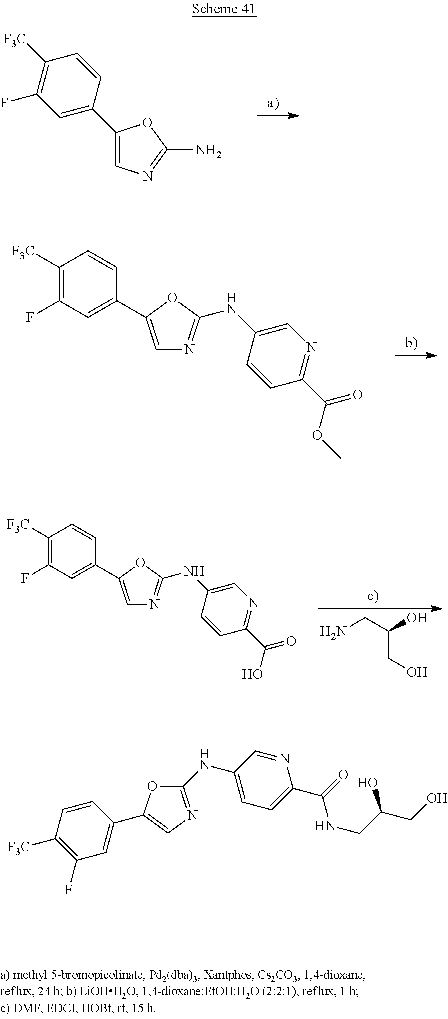 [SMILES: Nc1ncc(-c2ccc(C(F)(F)F)c(F)c2)o1 ; COC(=O)c1ccc(Nc2ncc(-c3ccc(C(F)(F)F)c(F)c3)o2)cn1 ; O=C(O)c1ccc(Nc2ncc(-c3ccc(C(F)(F)F)c(F)c3)o2)cn1 ; NC[C@@H](O)CO ; O=C(NC[C@@H](O)CO)c1ccc(Nc2ncc(-c3ccc(C(F)(F)F)c(F)c3)o2)cn1]

5-(3-Fluoro-4-(trifluoromethyl)phenyl)oxazol-2-amine (0.198 g, 0.804 mmol), methyl 5-bromopicolinate (0.208 g, 0.964 mmol), Cs₂CO₃ (0.393 g, 1.205 mmol), Pd₂(dba)₃ (0.037 g, 0.0402 mmol), Xantphos (0.0465 g, 0.0803 mmol) and anhydrous 1,4-dioxane (10 mL) were added into a dry RBF. The mixture was degassed, filled up with N_{2(g)}(×5) and was refluxed for 24 h under N_{2(g)}. The reaction mixture was quenched with aq. NaHCO₃ solution (8 mL) and diluted with EtOAc (10 mL). The organic layer was extracted with EtOAc (3×10 mL). The combined organic fractions were dried over MgSO₄, filtered and concentrated under reduced pressure. The residue was chromatographed on silica gel eluting with 50% EtOAc in DCM to afford methyl 5-((5-(3-fluoro-4-(trifluoromethyl)phenyl)oxazol-2-yl)amino)picolinate as light brown solid (0.227 g, 74%). ¹H NMR (400 MHz, DMSO-d₆) δ 11.30 (s, 1H), 8.83 (d, J=2.6 Hz, 1H), 8.31 (dd, J=8.7, 2.6 Hz, 1H), 8.09 (d, J=8.7 Hz, 1H), 7.89 (s, 1H), 7.86 (t, J=8.1 Hz, 1H), 7.60 (d, J=8.2 Hz, 1H), 3.85 (s, 3H). ¹³C NMR (101 MHz, DMSO-d₆) δ 166.97, 164.82, 156.41, 139.87, 138.60, 136.99, 132.37, 131.71, 131.59, 128.66, 126.94, 125.97, 122.85, 118.66, 52.07. LCMS R_{f}(min)=3.794, MS m/z=381.9 [M+H]⁺.

A solution of LiOH·H₂O (0.0387 g, 0.922 mmol) in H₂O (1 mL) was added dropwise to a solution of methyl 5-((5-(3-fluoro-4-(trifluoromethyl)phenyl)oxazol-2-yl)amino)picolinate (0.352 g, 0.922 mmol) in 1,4-dioxane (2 mL) and EtOH (2 mL). The mixture was stirred at 80° C. for 1 h. After this period of time the contents were evaporated to dryness. The residue was washed with Et₂O (15 mL), filtered off and dried under vacuum to afford the title compound as white solid (0.258 g, 76%) which was used in next step without further purification. ¹H NMR (400 MHz, DMSO-d₆) δ 8.58 (d, J=2.5 Hz, 1H), 8.14 (dd, J=8.5, 2.6 Hz, 1H), 7.88 (d, J=8.6 Hz, 1H), 7.81-7.74 (m, 2H), 7.60 (d, J=12.2 Hz, 1H), 7.50 (d, J=8.1 Hz, 1H). ¹³C NMR (101 MHz, DMSO-d₆) δ 168.60, 156.61, 142.90, 142.22, 137.98, 137.23, 134.11, 128.40, 126.97, 123.99, 123.72, 122.56, 121.29, 118.56, 110.47. LCMS R_{f}(min)=4.702, MS m/z=367.9 [M+H]⁺.

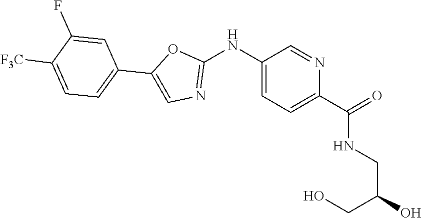 [SMILES: O=C(NC[C@@H](O)CO)c1ccc(Nc2ncc(-c3ccc(C(F)(F)F)c(F)c3)o2)cn1]

5-((5-(3-Fluoro-4-(trifluoromethyl)phenyl)oxazol-2-yl)amino)picolinic acid (0.12 g, 0.327 mmol), HOBt (0.046 g, 0.392 mmol), EDCl·HCl (0.069 g, 0.360 mmol) were added into a dry RBF and stirred with anhydrous DMF (6.5 mL) for 3 hours with continuous flow of N_{2(g)}. Then, (R)-3-aminopropane-1,2-diol (0.036 g, 0.392 mmol) was added and stirring was continued for 16 h. After this period of time the reaction was quenched with aq. NaHCO₃ solution (8 mL) and diluted with EtOAc (10 mL). The organic layer was extracted with EtOAc (3×10 mL) and the combined organics were dried over MgSO₄. The volatiles were removed under reduced pressure and the residue was subjected to preparative HPLC using 0.1% TFA in H₂O and 0.1% TFA in ACN as eluents to yield (R)—N-(2,3-dihydroxypropyl)-5-((5-(3-fluoro-4-(trifluoromethyl)phenyl)oxazol-2-yl)amino)picolinamide as white solid (0.086 g, 60%, 100% purity). ¹H NMR (400 MHz, DMSO-d₆) δ 11.18 (s, 1H), 8.80 (d, J=2.6 Hz, 1H), 8.46 (t, J=5.9 Hz, 1H), 8.30 (dd, J=8.6, 2.6 Hz, 1H), 8.05 (d, J=8.6 Hz, 1H), 7.88 (s, 1H), 7.85 (d, J=8.0 Hz, 1H), 7.73 (d, J=12.0 Hz, 1H), 7.59 (d, J=8.3 Hz, 1H), 4.95 (d, J=4.9 Hz, 1H), 4.66 (t, J=5.8 Hz, 1H), 3.63 (m, 1H), 3.50 (m, 1H), (the resonance of 2 protons overlapped with H₂O, and not assigned), 3.23 (m, 1H). ¹³C NMR (101 MHz, DMSO-d₆) δ 163.60, 156.61, 142.90, 142.22, 137.98, 137.23, 134.11, 128.40, 126.97, 123.99, 123.72, 122.56, 121.29, 118.56, 110.71, 110.47, 70.20, 63.94, 42.34. LCMS R_{f}(min)=3.266, MS m/z=440.9 [M+H]⁺. HRMS (ESI) calcd for C₁₉H₁₇F₄N₄O₄⁺ [M+H]⁺ 441.118, found 441.1197.

### 45. 5-((5-(3-Fluoro-4-(trifluoromethyl)phenyl)oxazol-2-yl)amino)-N′-hydroxypicolinimidamide (Scheme 42)

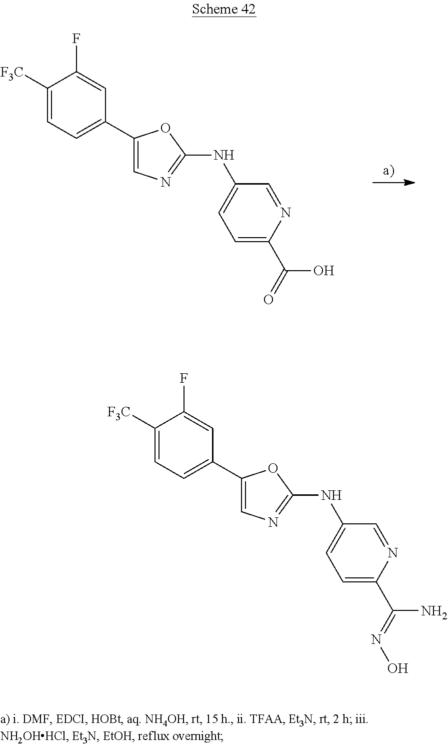 [SMILES: O=C(O)c1ccc(Nc2ncc(-c3ccc(C(F)(F)F)c(F)c3)o2)cn1 ; N/C(=N\O)c1ccc(Nc2ncc(-c3ccc(C(F)(F)F)c(F)c3)o2)cn1]

5-((5-(3-Fluoro-4-(trifluoromethyl)phenyl)oxazol-2-yl)amino)picolinic acid (0.093 g, 0.253 mmol) was suspended in DMF (3.0 mL), then EDCI (1.3 equiv.) and HOBt (1.4 equiv.) were added. The resulting mixture was stirred at rt for 3 h. Concentrated aqueous ammonia solution (1 mL) was added and stirring was continued overnight. The reaction mixture was concentrated under reduced pressure, and the residue was purified on preparative HPLC to provide (5-((5-(3-fluoro-4-(trifluoromethyl)phenyl)oxazol-2-yl)amino)picolinamide (LCMS R_{f}(min)=3.723, MS m/z=366.9 [M+H]⁺), which was suspended in anhydrous THF. Et₃N (3 equiv.) was added followed by TFAA (1.5 equiv.). The resulting solution was stirred at rt for 2 h, and the volatile solvents were removed in vacuo. The crude 5-((5-(3-fluoro-4-(trifluoromethyl)phenyl)oxazol-2-yl)amino)picolinonitrile was dried under high vacuum, then redissolved in absolute EtOH. NH₂OH·HCl (5 equivalents), and Et₃N (5 equivalents) were added, and the resultant reaction mixture was stirred at reflux overnight. All volatile solvents were removed and the residue purified on preparative HPLC to provide 5-((5-(3-fluoro-4-(trifluoromethyl)-phenyl)oxazol-2-yl)amino)-N′-hydroxypicolinimidamide (0.023 g; 21.35% yield over three steps, corrected for 0.5 molar equivalent of dioxane present). ¹H NMR (401 MHz, DMSO-d₆) δ 11.36 (s, 1H), 10.92-10.88 (br, 1H), 8.87 (s, 1H), 8.30 (d, J=8.7 Hz, 1H), 8.03 (d, J=8.6 Hz, 1H), 7.87 (s, 1H), 7.84 (t, J=8.0 Hz, 1H), 7.70 (d, J=12.0 Hz, 1H), 7.58 (d, J=8.4 Hz, 1H). ¹³C NMR (101 MHz, DMSO-d₆) δ 161.09, 158.56, 156.88, 142.85, 138.85, 138.75, 134.51, 134.41, 128.85, 127.37, 127.14, 124.44, 123.88, 123.16, 121.75, 119.08, 119.04, 115.15, 115.03, 114.83, 114.71, 111.22, 110.99. LCMS R_{f} (min)=3.782, MS m/z=381.9 [M+H]⁺. HRMS (ESI) calcd for C₁₆H₁₂F₄N₅O₂⁺ [M+H]⁺ 382.0922, found 382.0907.

### 46. N′-hydroxy-5-((5-(5-(trifluoromethyl)pyridin-2-yl)oxazol-2-yl)amino)-picolinimidamide (Scheme 43)

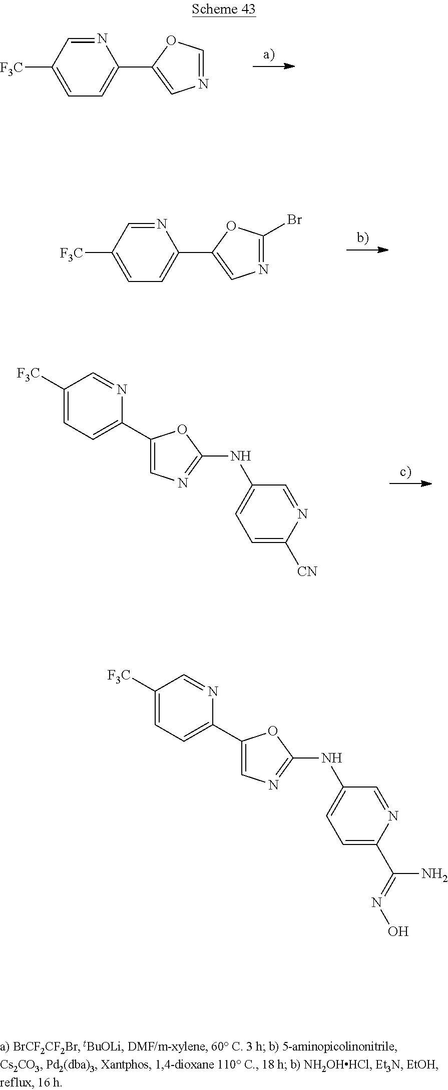 [SMILES: FC(F)(F)c1ccc(-c2cnco2)nc1 ; FC(F)(F)c1ccc(-c2cnc(Br)o2)nc1 ; N#Cc1ccc(Nc2ncc(-c3ccc(C(F)(F)F)cn3)o2)cn1 ; N/C(=N\O)c1ccc(Nc2ncc(-c3ccc(C(F)(F)F)cn3)o2)cn1]

### Intermediate L—2-bromo-5-(5-(trifluoromethyl)pyridin-2-yl)oxazole

BrCF₂CF₂Br (1.71 g, 6.6 mmol) and ^{t}BuOLi (0.53 g, 6.6 mmol) were added to a solution of 5-(5-(trifluoromethyl)pyridin-2-yl)oxazole (0.70 g, 3.3 mmol) in DMF/m-xylene (5/5 mL). The resulting mixture was stirred at 60° C. for 3 h and quenched with saturated NaHCO₃ (10 mL). The aqueous phase was extracted with EtOAc (20 mL×2). The combined organic phases were dried over Na₂SO₄, filtered, and concentrated to give a brown oil that was subjected to flash chromatography on silica gel (3%˜10%, EtOAc/hexane). Collection of the appropriate fractions provided 2-bromo-5-(5-(trifluoromethyl)pyridin-2-yl)oxazole as a white solid (0.68 g, 70% yield). ¹H NMR (400 MHz, CDCl₃) δ 8.86 (m, 1H), 8.00 (dd, J=8.3, 1.7 Hz, 1H), 7.75 (s, 1H), 7.72 (d, J=8.3 Hz, 1H). MS m/z=292.9 [M+H]⁺.

An oven-dried RBF was charged with Pd₂(dba)₃ (0.025 g, 0.04 equiv.), Xantphos (0.031 g, 0.08 equiv.), 2-bromo-5-(5-(trifluoromethyl)pyridin-2-yl)oxazole (Intermediate L) (0.2 g, 0.68 mmol), Cs₂CO₃ (0.332 g, 1.5 equiv.), 5-aminonicotinonitrile (0.405 g, 3.4 mmol, 5 equiv.) and 1,4-dioxane (10 mL). Vacuum was applied briefly to the reaction flask followed by backfilling with N₂ and the procedure was repeated five times. The mixture was then heated to 110° C. and stirred for 18 h. The reaction mixture was poured into water and extracted with EtOAc (5×). The combined organic solvents were dried over MgSO₄ and evaporated to dryness. The residue was purified on silica gel using mixtures of DCM and EtOAc to provide 5-((5-(5-(trifluoromethyl)pyridin-2-yl)oxazol-2-yl)amino)picolinonitrile (0.127 g, 56.36% yield). ¹H NMR (401 MHz, DMSO-d₆) δ 11.59 (s, 1H), 8.93 (d, J=1.0 Hz, 1H), 8.85 (d, J=2.4 Hz, 1H), 8.32 (dd, J=8.7, 2.6 Hz, 1H), 8.26 (dd, J=8.6, 2.1 Hz, 1H), 7.99 (d, J=8.7 Hz, 1H), 7.93 (s, 1H), 7.81 (d, J=8.4 Hz, 1H). ¹³C NMR (101 MHz, DMSO-d₆) δ 157.07, 150.25, 147.07, 144.32, 140.75, 139.28, 135.28, 130.30, 129.31, 125.56, 124.60, 123.47, 123.14, 118.42, 118.38. LCMS R_{f}(min)=2.978, MS m/z=332.1 [M+H]⁺.

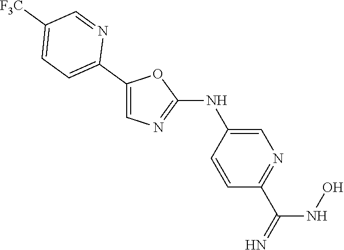 [SMILES: N=C(NO)c1ccc(Nc2ncc(-c3ccc(C(F)(F)F)cn3)o2)cn1]

5-((5-(5-(Trifluoromethyl)pyridin-2-yl)oxazol-2-yl)amino)picolinonitrile (0.127 g, 0.383 mmol) was suspended in EtOH (10 mL), then NH₂OH·HCl (0.213 g, 3.067, mmol, 8 equiv.) was added and approximately half of the EtOH was removed in vacuo. Et₃N (0.42 mL, 3.067, mmol, 8 equiv.) was added and the resulting reaction mixture was stirred at reflux overnight. The volatile solvents were removed and the resulting solids were suspended in water, were collected by filtering then washing well with water. The crude solid was purified on preparative HPLC to provide N-hydroxy-5-((5-(5-(trifluoromethyl)pyridin-2-yl)oxazol-2-yl)amino)picolinimidamide (0.106 g; 75.9%). ¹H NMR (401 MHz, DMSO-d₆) δ 11.50 (s, 1H), 10.97-10.88 (brs, 1H), 8.95-8.93 (m, 1H), 8.89 (d, J=2.5 Hz, 1H), 8.6-8.2 (br, 1H), 8.32 (dd, J=8.8, 2.6 Hz, 1H), 8.27 (dd, J=8.7, 2.1 Hz, 1H), 8.04 (d, J=8.8 Hz, 1H), 7.94 (s, 1H), 7.81 (d, J=8.4 Hz, 1H). ¹³C NMR (101 MHz, DMSO-d₆) δ 158.42, 157.39, 154.87, 150.31, 147.06, 144.17, 138.92, 138.84, 135.26, 129.41, 125.57, 123.96, 123.38, 123.33, 123.06, 122.87, 118.28. LCMS R_{f} (min)=2.865, MS m/z=365.1 [M+H]⁺. HRMS (ESI) calcd for C₁₅H₁₂F₃N₆O₂⁺ [M+H]⁺ 365.0968, found 365.0974.

### 47. 6-((5-(4-Fluorophenyl)oxazol-2-yl)amino)-N′-hydroxypyridazine-3-carboximidamide (Scheme 44)

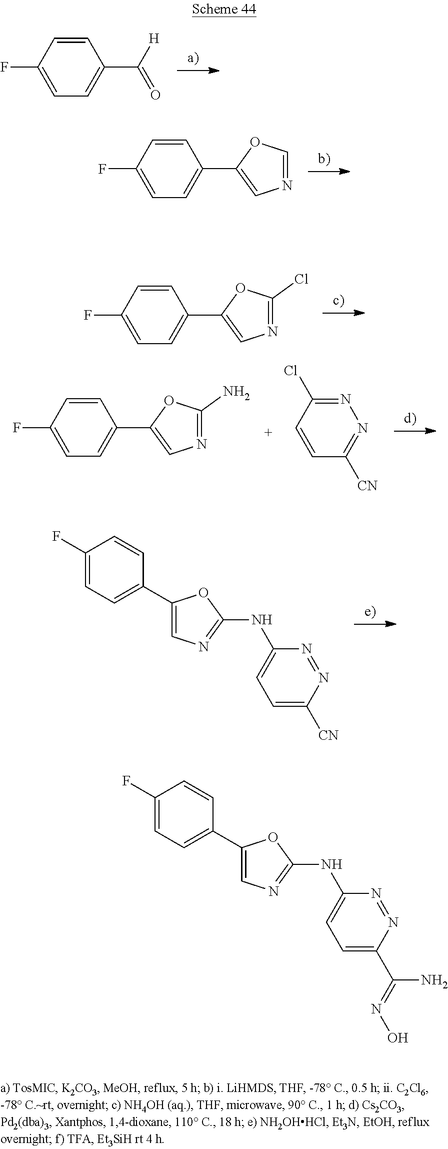

4-Fluorobenzaldehyde (2.00 g, 16.11 mmol) and K₂CO₃ (2.67 g, 19.33 mmol) were added to a solution of p-toluenesulfonylmethyl isocyanide (3.46 g, 17.73 mmol) in MeOH (30 mL) at rt. The resulting mixture was then refluxed for 5 h under nitrogen before being concentrated under reduced pressure. The residue was partitioned between Et₂O and H₂O. The aqueous phase was extracted with Et₂O (50 mL×2), and the combined organic phases were dried over MgSO₄, filtered and concentrated to give a light yellow oil that was subjected to flash chromatography on silica gel (3%˜20%, EtOAc/hexane). Collection of the appropriate fractions provided 5-(4-fluorophenyl)oxazole as a white solid. (2.16 g, 82% yield). ¹H NMR (400 MHz, CDCl₃) δ) δ 7.90 (s, 1H), 7.58-7.63 (m, 2H), 7.28 (s, 1H), 7.07-7.13 (m, 2H). MS m/z=164.1 [M+H]⁺.

LiHMDS (1.0 M in THF, 6.58 mL, 6.58 mmol) was added to a solution of 5-(4-fluorophenyl)oxazole (0.98 g, 5.98 mmol) in dry THF (6 mL) at −78° C. under nitrogen atmosphere. After a further 0.5 h, a solution of C₂Cl₆ (2.12 g, 8.97 mmol) in THF (5 mL) was treated. The resulting reaction mixture was stirred at −78° C. for another 2 h and allowed to warm to rt over 14 h. The reaction was quenched with saturated NaHCO₃ (10 mL). The aqueous phase was extracted with EtOAc (30 mL×2). The combined organic phases were dried over Na₂SO₄, filtered, and concentrated to give a brown oil that was subjected to flash chromatography on silica gel (2% 10%, EtOAc/hexane). Collection of the appropriate fractions provided 2-chloro-5-(4-fluorophenyl)oxazole as a white solid (1.06 g, 90% yield). ¹H NMR (400 MHz, CDCl₃) δ 7.55-7.60 (m, 2H), 7.23 (s, 1H), 7.10-7.15 (m, 2H). MS m/z=198.0 [M+H]⁺.

NH₄OH (28-30% NH₃ basis aqueous solution, 8 mL) was added to a solution of 2-chloro-5-(4-fluorophenyl)oxazole (0.60 g, 3.04 mmol) in THF (1.5 mL) at rt. The resulting reaction mixture was irradiated under microwave for 1 h at 90° C. The solid was filtered and washed with DCM (5 mL×2), providing 5-(4-fluorophenyl)oxazol-2-amine as a white solid (0.52 g, 96% yield). ¹H NMR (400 MHz, DMSO-d₆) δ 7.47-7.50 (m, 2H), 7.18-7.23 (m, 2H), 7.15 (s, 1H), 6.82 (s, 2H). MS m/z=179.0 [M+H]⁺.

An oven-dried RBF was charged with Pd₂(dba)₃ (0.044 g, 0.05 equiv.), Xantphos (0.055 g, 0.10 equiv.), 5-(4-fluorophenyl)oxazol-2-amine (0.17 g, 0.954 mmol), Cs₂CO₃ (0.466 g, 1.5 equiv.), 6-chloropyridazine-3-carbonitrile (0.146 g, 1.05 mmol, 1.1 equiv.) and 1,4-dioxane (5 mL). Vacuum was applied briefly to the reaction flask followed by backfilling with N₂ and the procedure was repeated five times. The mixture was then heated to 110° C. and stirred for 18 h. The reaction mixture was poured into water and extracted with EtOAc (5×). The combined organic solvents were dried over MgSO₄ and evaporated to dryness. The residue was purified on silica gel using mixtures of DCM and EtOAc to provide 6-((5-(4-fluorophenyl)oxazol-2-yl)amino)pyridazine-3-carbonitrile (0.181 g, 67.44% yield). ¹H NMR (401 MHz, DMSO-d₆) δ 12.37 (br, 1H), 8.41 (s, 1H), 8.22 (d, J=9.4 Hz, 1H), 7.67 (dd, J=8.7, 5.4 Hz, 2H), 7.59 (s, 1H), 7.32 (t, J=8.9 Hz, 2H). ¹³C NMR (101 MHz, DMSO-d₆) δ 163.26, 160.82, 133.39, 125.81, 125.73, 124.53, 124.50, 117.07, 116.75, 116.53. LCMS R_{f}(min)=3.185. MS m/z=282.1 [M+H]⁺.

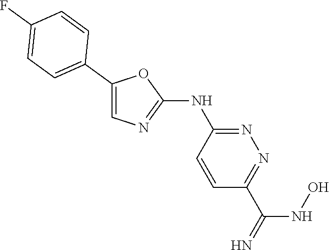

6-((5-(4-Fluorophenyl)oxazol-2-yl)amino)pyridazine-3-carbonitrile (0.152 g, 0.54 mmol) was suspended in EtOH (12 mL), then NH₂OH·HCl (0.301 g, 4.323 mmol, 8 equiv.) was added and approximately half of the EtOH was removed in vacuo. Et₃N (0.59 mL, 4.323 mmol, 8 equiv.) was added and the resulting reaction mixture was stirred at reflux overnight. The volatile solvents were removed and the resulting solids were suspended in water, collected by filtering then washing well with water. The crude solid was purified on preparative HPLC to provide 6-((5-(4-fluorophenyl)oxazol-2-yl)amino)-N-hydroxypyridazine-3-carboximidamide (0.075 g; 43.4%). ¹H NMR (401 MHz, DMSO-d₆) δ 10.59 (s, 1H), 8.28 (d, J=8.9 Hz, 1H), 8.08 (d, J=9.5 Hz, 1H), 7.68 (dd, J=8.7, 5.4 Hz, 2H), 7.59 (s, 1H), 7.32 (t, J=8.9 Hz, 2H), ¹³C NMR (101 MHz, DMSO-d₆) δ 163.19, 160.75, 158.51, 156.23, 144.92, 127.54, 125.71, 125.63, 124.69, 121.95, 116.73, 116.51. LCMS R_{f}(min)=2.935, MS m/z=315.1 [M+H]⁺. HRMS (ESI) calcd for C₁₄H₁₂FN₆O₂⁺ [M+H]⁺ 315.1, found 315.0991.

### 48. 6-((5-(3,4-Difluorophenyl)oxazol-2-yl)amino)-N′-hydroxypyridazine-3-carboximidamide (Scheme 45)

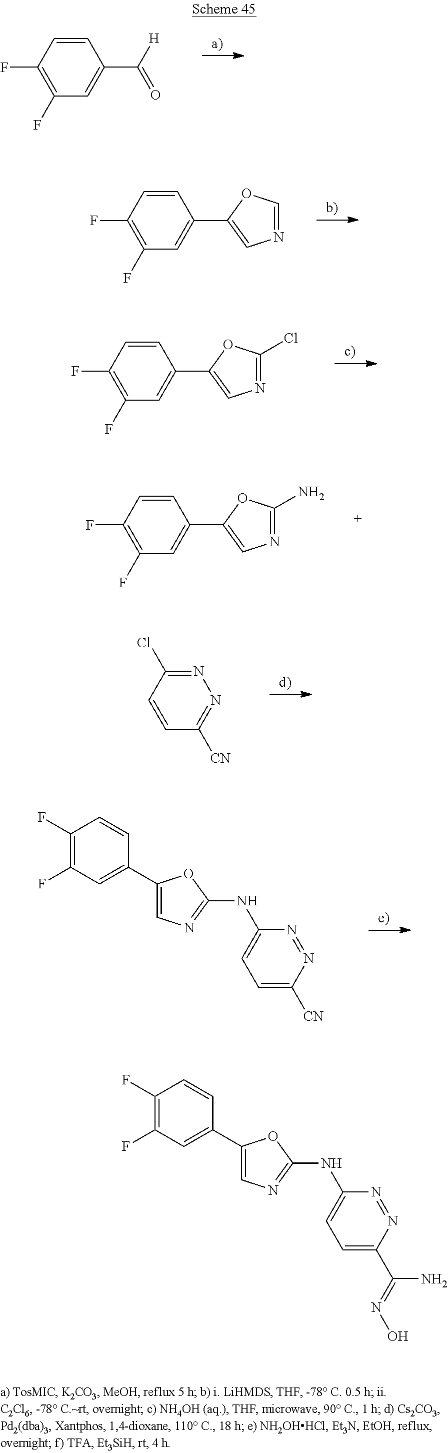

3,4-Difluorobenzaldehyde (2.00 g, 14.1 mmol) and K₂CO₃ (2.33 g, 16.9 mmol) were added to a solution of TosMIC (3.02 g, 15.5 mmol) in MeOH (30 mL) at rt. The resulting mixture was then refluxed for 5 h under nitrogen before being concentrated under reduced pressure. The residue was partitioned between Et₂O and H₂O. The aqueous phase was extracted with Et₂O (50 mL×2), and the combined organic phases were dried over MgSO₄, filtered and concentrated to give a light yellow oil that was subjected to flash chromatography on silica gel (3%-20%, EtOAc/hexane). Collection of the appropriate fractions provided 5-(3,4-difluorophenyl)oxazole) as a white solid. (2.25 g, 88% yield). ¹H NMR (400 MHz, CDCl₃) δ) δ 7.55-7.60 (m, 2H), 7.23 (s, 1H), 7.09-7.15 (m, 2H), MS m/z=182.0 [M+H]⁺.

LiHMDS (1.0 M in THF, 8.46 mL, 8.46 mmol) was added to a solution of 5-(3,4-difluorophenyl)oxazole (1.40 g, 7.69 mmol) in dry THF (10 mL) at −78° C. under nitrogen atmosphere. After a further 0.5 h, a solution of C₂Cl₆ (2.73 g, 11.5 mmol) in THF (5 mL) was treated. The resulting reaction mixture was stirred at −78° C. for another 2 h and allowed to warm to rt over 14 h. The reaction was quenched with saturated NaHCO₃ (10 mL). The aqueous phase was extracted with EtOAc (40 mL×2). The combined organic phases were dried over Na₂SO₄, filtered, and concentrated to give a brown oil that was subjected to flash chromatography on silica gel (2%˜10%, EtOAc/hexane). Collection of the appropriate fractions provided 2-chloro-5-(3,4-difluorophenyl)oxazole as a white solid (1.41 g, 85% yield). ¹H NMR (400 MHz, CDCl₃) δ 7.47 (m, 1H), 7.39 (m, 1H), 7.32 (s, 1H), 7.31 (m, 1H). MS m/z=216.0 [M+H]⁺.

NH₄OH (28-30% NH₃ basis aqueous solution, 12 mL) was added to a solution of 2-chloro-5-(3,4-difluorophenyl)oxazole (1.10 g, 4.42 mmol) in THF (2.0 mL) at rt. The resulting reaction mixture was irradiated under microwave for 1 h at 90° C. The solid was filtered and washed with DCM (5 mL×2), providing 5-(3,4-difluorophenyl)oxazol-2-amine as a white solid (0.82 g, 95% yield). ¹H NMR (400 MHz, DMSO-d₆) δ 7.38-7.49 (m, 2H), 7.25 (s, 2H), 6.93 (s, 2H). MS m/z=197.0 [M+H]⁺.

An oven-dried RBF was charged with Pd₂(dba)₃ (0.058 g, 0.05 equiv.), Xantphos (0.074 g, 0.10 equiv.), 5-(3,4-difluorophenyl)oxazol-2-amine (0.25 g, 1.274 mmol), Cs₂CO₃ (0.622 g, 1.5 equiv.), 6-chloropyridazine-3-carbonitrile (0.196 g, 1.40 mmol, 1.1 equiv.) and 1,4-dioxane (7 mL). Vacuum was applied briefly to the reaction flask followed by backfilling with N₂ and the procedure was repeated five times. The mixture was then heated to 110° C. and stirred for 18 h. The reaction mixture was poured into water and extracted with EtOAc (5×). The combined organic solvents were dried over MgSO₄ and evaporated to dryness. The residue was purified on silica gel using mixtures of DCM and EtOAc to provide 6-((5-(3,4-difluorophenyl)oxazol-2-yl)amino)pyridazine-3-carbonitrile (0.194 g, 50.9% yield). ¹H NMR (401 MHz, DMSO-d₆) δ 12.44-12.38 (br s, 1H), 8.41 (d, J=8.9 Hz, 1H), 8.23 (d, J=9.4 Hz, 1H), 7.74-7.69 (m, 1H), 7.68 (s, 1H), 7.56 (dt, J=10.5, 8.5 Hz, 1H), 7.48-7.43 (m, 1H). ¹³C NMR (101 MHz, DMSO-d₆) δ 156.61, 154.90, 151.62, 151.49, 150.43, 149.18, 149.05, 148.10, 133.42, 125.44, 120.44, 119.13, 118.95, 117.04, 112.84, 112.65. LCMS R_{f}(min)=3.061, MS m/z=300.0 [M+H]⁺.

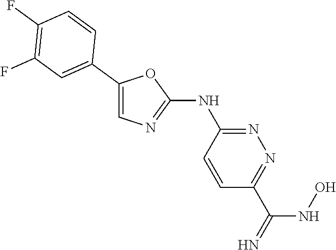

6-((5-(3,4-Difluorophenyl)oxazol-2-yl)amino)pyridazine-3-carbonitrile (0.095 g, 0.317 mmol) was suspended in EtOH (12 mL), then NH₂OH·HCl (0.177 g, 2.54 mmol, 8 equiv.) was added and approximately half of the EtOH was removed in vacuo. Et₃N (0.347 mL, 2.54 mmol, 8 equiv.) was added and the resulting reaction mixture was stirred at reflux overnight. The volatile solvents were removed and the resulting solids were suspended in water, collected by filtering then washing well with water. The crude solid was purified on preparative HPLC to provide 6-((5-(3,4-difluorophenyl)oxazol-2-yl)amino)-N-hydroxypyridazine-3-carboximidamide (0.083 g; 78.67%). ¹H NMR (401 MHz, DMSO-d₆) δ 11.85 (brs, 1H), 10.16 (brs, 1H), 8.31-7.47 (m, 6H), 5.97 (s, 2H). LCMS R_{f} (min)=2.848, MS m/z=333.1 [M+H]⁺. HRMS (ESI) calcd for C₁₄H₁₁F₂N₆O₂⁺ [M+H]⁺ 333.0906, found 333.0908.

### 49. 6-((5-(5-Fluoropyridin-2-yl)oxazol-2-yl)amino)-N′-hydroxypyridazine-3-carboximidamide (Scheme 46)

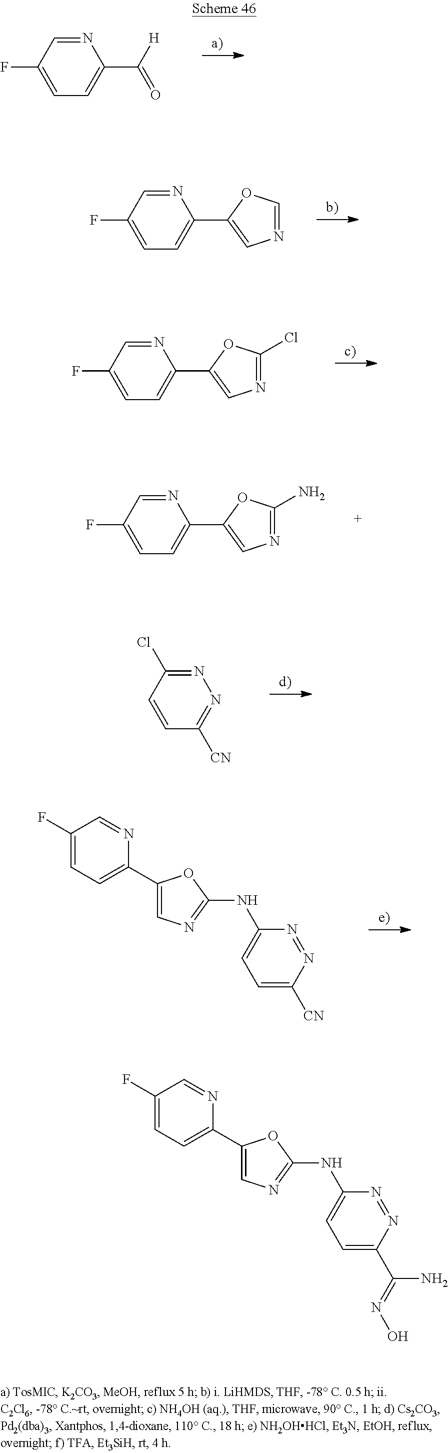

5-Fluoropicolinaldehyde (1.20 g, 9.60 mmol) and K₂CO₃ (1.60 g, 11.5 mmol) were added to a solution of p-toluenesulfonylmethyl isocyanide (2.06 g, 10.6 mmol) in MeOH (20 mL) at rt. The resulting mixture was then refluxed for 5 h under nitrogen before being concentrated under reduced pressure. The residue was partitioned between Et₂O and H₂O. The aqueous phase was extracted with Et₂O (20 mL×2), and the combined organic phases were dried over MgSO₄, filtered and concentrated to give a light yellow oil that was subjected to flash chromatography on silica gel (3%˜20%, EtOAc/hexane). Collection of the appropriate fractions provided 5-(5-fluoropyridin-2-yl)oxazole as a white solid. (1.39 g, 88% yield). ¹H NMR (400 MHz, CDCl₃) δ) δ 8.49 (d, J=2.8 Hz, 1H, split by F), 7.95 (s, 1H), 7.66-7.69 (m, 1H), 7.64 (s, 1H), 7.48 (td, J=8.3, 2.8 Hz, 1H). MS m/z=165.1 [M+H]⁺.

LiHMDS (1.0 M in THF, 5.18 mL, 5.18 mmol) was added to a solution of 5-(5-fluoropyridin-2-yl)oxazole (0.80 g, 4.93 mmol) in dry THF (5 mL) at −78° C. under nitrogen atmosphere. After a further 0.5 h, a solution of C₂Cl₆ (1.52 g, 6.41 mmol) in THF (5 mL) was treated. The resulting reaction mixture was stirred at −78° C. for another 2 h and allowed to warm to rt over 14 h. The reaction was quenched with saturated NaHCO₃ (10 mL). The aqueous phase was extracted with EtOAc (30 mL×2). The combined organic phases were dried over Na₂SO₄, filtered, and concentrated to give a brown oil that was subjected to flash chromatography on silica gel (2% 10%, EtOAc/hexane). Collection of the appropriate fractions provided 2-chloro-5-(5-fluoropyridin-2-yl)oxazole as a white solid (0.82 g, 84% yield). ¹H NMR (400 MHz, CDCl₃) δ 7.47 (d, J=2.8 Hz, 1H, split by F), 7.59-7.63 (m, 1H), 7.56 (s, 1H), 7.48 (td, J=8.0 and 2.8 Hz, 1H). MS m/z=198.9 [M+H]⁺.

NH₄OH (28-30% NH₃ basis aqueous solution, 4 mL) was added to a solution 2-chloro-5-(5-fluoropyridin-2-yl)oxazole (0.20 g, 1.02 mmol) in THF (0.5 mL) at rt. The resulting reaction mixture was irradiated under microwave for 1 h at 90° C. The solid was filtered and washed with DCM (3 mL×2), providing 5-(5-fluoropyridin-2-yl)oxazol-2-amine as a white solid (0.18 g, 97% yield). ¹H NMR (400 MHz, DMSO-d₆) δ 8.48 (s, 1H), 7.72 (m, 1H), 7.47 (m, 1H), 7.30 (s 1H), 7.05 (s, 2H). MS m/z=180.1 [M+H]⁺.

An oven-dried RBF was charged with Pd₂(dba)₃ (0.044 g, 0.05 equiv.), Xantphos (0.055 g, 0.10 equiv.), 5-(5-fluoropyridin-2-yl)oxazol-2-amine (0.17 g, 0.948 mmol), Cs₂CO₃ (0.464 g, 1.5 equiv.), 6-chloropyridazine-3-carbonitrile (0.146 g, 1.04 mmol, 1.1 equiv.) and 1,4-dioxane (5 mL). Vacuum was applied briefly to the reaction flask followed by backfilling with N₂ and the procedure was repeated five times. The mixture was then heated to 110° C. and stirred for 18 h. The reaction mixture was poured into water and extracted with EtOAc (5×). The combined organic solvents were dried over MgSO₄ and evaporated to dryness. The residue was purified on silica gel using mixtures of DCM and EtOAc to provide 6-((5-(5-fluoropyridin-2-yl)oxazol-2-yl)amino)pyridazine-3-carbonitrile (0.053 g, 19.78% yield). ¹H NMR (401 MHz, DMSO-d₆) δ 12.51 (s, 1H), 8.61 (d, J=2.8 Hz, 1H), 8.46 (d, J=9.0 Hz, 1H), 8.25 (d, J=9.4 Hz, 1H), 7.86 (td, J=8.7, 2.9 Hz, 1H), 7.72 (m, J=4.1 Hz, 2H). ¹³C NMR (101 MHz, DMSO-d₆) δ 159.74, 157.21, 156.60, 145.29, 143.53, 138.57, 138.33, 133.46, 125.05, 124.86, 120.38, 120.34, 117.04. LCMS R_{f}(min)=2.892, MS m/z=283.1 [M+H]⁺.

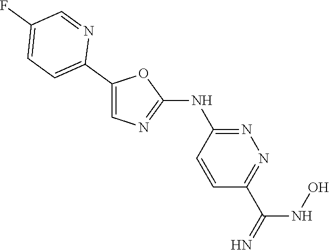

6-((5-(5-Fluoropyridin-2-yl)oxazol-2-yl)amino)pyridazine-3-carbonitrile (0.05 g, 0.177 mmol) was suspended in EtOH (7 mL), then NH₂OH·HCl (0.099 g, 1.417 mmol, 8 equiv.) was added and approximately half of the EtOH was removed in vacuo. Et₃N (0.193 mL, 1.417 mmol, 8 equiv.) was added and the resulting reaction mixture was stirred at reflux overnight. The volatile solvents were removed and the resulting solids were suspended in water, collected by filtering then washing well with water, EtOH, and Et₂O to provide 6-((5-(5-fluoropyridin-2-yl)oxazol-2-yl)amino)-N′-hydroxypyridazine-3-carboximidamide (0.041 g; 73.4%). ¹H NMR (401 MHz, DMSO-d₆) δ 11.97-11.87 (br, 1H), 10.17 (s, 1H), 8.60 (d, J=2.7 Hz, 1H), 8.29 (br, 1H), 8.02 (d, J=8.9 Hz, 1H), 7.85 (td, J=8.7, 2.8 Hz, 1H), 7.72-7.69 (m, 1H), 7.67 (s, 1H), 5.98 (s, 2H). ¹³C NMR (101 MHz, DMSO-d₆) δ 159.58, 157.07, 144.72, 143.85, 138.50, 138.26, 124.99, 124.79, 120.06, 120.02. LCMS R_{f}(min)=2.439, MS m/z=316.1 [M+H]⁺. HRMS (ESI) calcd for C₁₃H₁₁FN₇O₂⁺ [M+H]⁺ 316.0953, found 316.0954.

### 50. N,5-Dihydroxy-2-((5-(4-(trifluoromethyl)phenyl)oxazol-2-yl)amino)isonicotinamide-amide (Scheme 47)

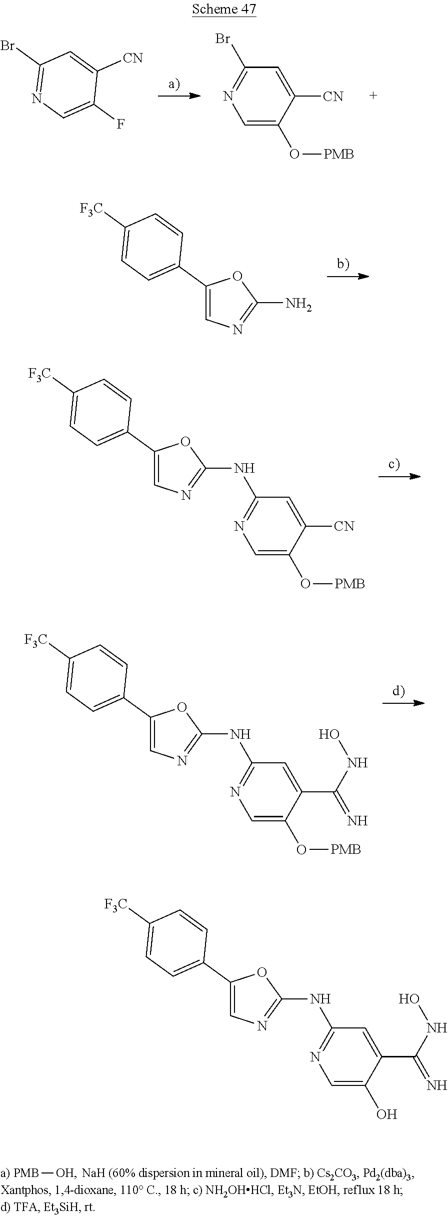

4-Methoxybenzyl alcohol (0.687 g, 4.975 mmol, 1.0 equivalent) was dissolved in dry DMF (12 mL), then NaH (60%, 0.119 g, 4.975 mmol, 1.0 equivalent)) added under nitrogen atmosphere at 0° C. After 15 minutes of stirring, the solution was transferred to a flask containing 2-bromo-5-fluoroisonicotinonitrile (1.0 g, 0.495 mmol) in dry DMF (12 mL) at 0° C. The resultant reaction mixture was stirred at rt for 1 h. LCMS indicated that all starting material had been consumed. The reaction mixture was poured into sat. aq. NaHCO₃ solution and the product extracted with DCM (5×). The combined organic solutions were dried over MgSO₄, and evaporated to dryness. The residue was purified on silica gel using neat toluene as eluents to provide 2-bromo-5-((4-methoxybenzyl)oxy)isonicotinonitrile (1.37 g, 86.27%). ¹H NMR (401 MHz, CDCl₃) δ 8.25 (s, 1H), 7.59 (d, J=0.4 Hz, 1H), 7.36-7.33 (m, 2H), 6.94-6.89 (m, 2H), 5.23 (s, 2H), 3.81 (d, J=3.3 Hz, 3H). ¹³C NMR (101 MHz, CDCl₃) δ 160.10, 154.31, 136.57, 131.84, 130.35, 129.26, 126.22, 114.38, 112.86, 112.73, 71.87, 55.36. LCMS R_{f}(min)=3.691, MS m/z=316.9/318.9 (M−H)⁻.

An oven-dried RBF was charged with Pd₂(dba)₃ (0.108 g, 0.15 equiv.), Xantphos (0.09 g, 0.2 equiv.), 5-(4-(trifluoromethyl)phenyl)oxazol-2-amine (Intermediate D) (0.215 g, 0.939 mmol, 1.2 equiv.), Cs₂CO₃ (0.383 g, 1.5 equiv.), 2-bromo-5-((4-methoxybenzyl)oxy)isonicotinonitrile (0.25 g, 0.783 mmol) and 1,4-dioxane (15 mL). Vacuum was applied briefly to the reaction flask followed by backfilling with N₂ and the procedure was repeated five times. The mixture was then heated to 110° C. and stirred for 18 h. LCMS indicated that the starting materials had been completely consumed. The reaction mixture was then poured into water and extracted with EtOAc (5×). The combined organic solvents were dried over MgSO₄ and evaporated to dryness. The residue was purified on silica gel using mixtures of DCM and EtOAc to provide 5-((4-methoxybenzyl)oxy)-2-((5-(4-(trifluoromethyl)phenyl)oxazol-2-yl)amino)isonicotinonitrile (0.051 g, 13.95% yield). ¹H NMR (401 MHz, DMSO-d₆) δ 11.33 (s, 1H), 8.63-8.42 (s, 1H), 8.31 (s, 1H), 7.80 (s, 4H), 7.74 (s, 1H), 7.43 (d, J=8.4 Hz, 2H), 6.95 (d, J=8.5 Hz, 2H), 5.28 (s, 2H), 3.77 (s, 3H). LCMS R_{f}(min)=3.93, MS m/z=467.1 [M+H]⁺.

5-((4-Methoxybenzyl)oxy)-2-((5-(4-(trifluoromethyl)phenyl)oxazol-2-yl)amino)isonicotinonitrile (0.043 g, 0.092 mmol) was suspended in EtOH (10 mL), then NH₂OH·HCl (0.051 g, 0.737 mmol) was added and approximately half of the EtOH was removed in vacuo. Et₃N (0.101 mL, 0.737 mmol) was added and the resulting reaction mixture was stirred at reflux overnight. LCMS indicated that the reaction was complete. The volatile solvents were removed and the resulting solids were suspended in water, collected by filtering then washing well with water. The solids were washed with Et₂O then dried under high vacuum to provide N-hydroxy-5-((4-methoxybenzyl)oxy)-2-((5-(4-(trifluoromethyl)phenyl)oxazol-2-yl)amino)isonicotinamidoxime (0.033 g, 71.66% yield). The product was used in the next step without further purification LCMS R_{f}(min)=4.733, MS m/z=499.9 [M+H]⁺.

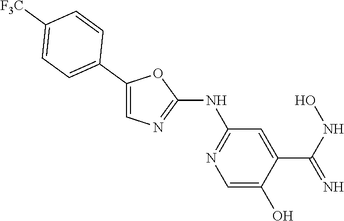 [SMILES: N=C(NO)c1cc(Nc2ncc(-c3ccc(C(F)(F)F)cc3)o2)ncc1O]

(N-hydroxy-5-((4-methoxybenzyl)oxy)-2-((5-(4-(trifluoromethyl)phenyl)oxazol-2-yl)amino)isonicotinamidoxime (33 mg, 0.066 mmol) was stirred in a mixture of TFA (2 mL) and Et₃SiH (0.1 mL) at rt for 4 h. The reaction mixture was evaporated to dryness. The residue was purified on preparative HPLC to provide N,5-dihydroxy-2-((5-(4-(trifluoromethyl)phenyl)oxazol-2-yl)amino)isonicotinamidoxime (5.0 mg, 19.95% yield). ¹H NMR (401 MHz, DMSO-d₆) δ 10.68 (s, 1H), 10.48 (s, 1H), 8.42 (s, 1H), 8.12 (s, 1H), 7.99 (s, 1H), 7.77 (s, 4H), 7.71 (s, 1H), 6.43 (s, 2H). ¹³C NMR (101 MHz, DMSO) δ 158.83, 158.52, 157.53, 152.33, 148.30, 144.76, 143.21, 136.99, 132.23, 128.77, 127.69, 127.37, 127.06, 126.74, 126.48, 126.44, 126.07, 125.77, 124.37, 123.26, 108.41. LCMS R_{f}(min)=3.62, MS m/z=380.1 [M+H]⁺. HRMS (ESI) calcd for C₁₆H₁₃F₃N₅O₃⁺ [M+H]⁺ 380.0965, found 380.0967.

### 51. 5-((5-(4-(Difluoromethoxy)phenyl)oxazol-2-yl)amino)-N′-hydroxy Picolinimidamide (Scheme 48)

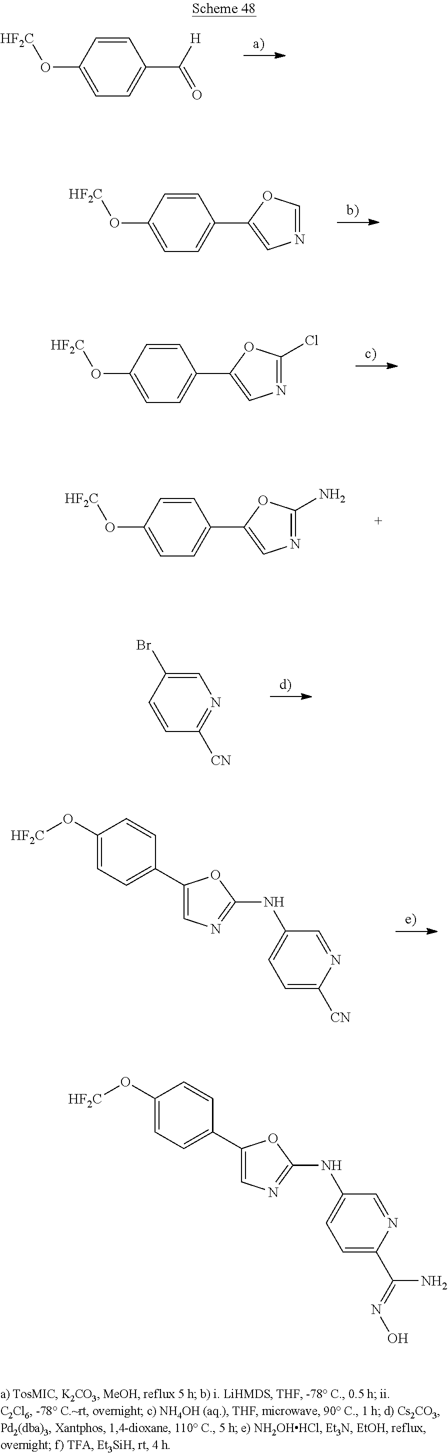

4-(Difluoromethoxy)benzaldehyde (3.0 g, 17.4 mmol) and K₂CO₃ (2.89 g, 20.9 mmol) were added to a solution of TosMIC (3.74 g, 19.2 mmol) in MeOH (40 mL) at rt. The resulting mixture was refluxed for 5 h under nitrogen before being concentrated under reduced pressure. The residue was partitioned between Et₂O and H₂O. The aqueous phase was extracted with Et₂O (40 mL×2), and the combined organic phases were dried over MgSO₄, filtered and concentrated to give a light yellow oil that was subjected to flash chromatography on silica gel (3%˜10%, EtOAc/hexane). Collection of the appropriated fractions provided 5-(4-(difluoromethoxy)phenyl)oxazole as a white solid (3.23 g, 88% yield). ¹H NMR (400 MHz, CDCl₃) δ 7.91 (s, 1H), 7.63 (d, J=8.0 Hz, 2H), 7.31 (s, 1H), 7.17 (d, J=8.0 Hz, 2H), 6.54 (t, J=72 Hz, 1H, split by F). MS m/z=212.1 [M+H]⁺.

LiHMDS (1.0 M in THF, 8.86 mL, 8.86 mmol) was added to a solution of 5-(4-(difluoromethoxy)phenyl)oxazole (1.70 g, 8.05 mmol) in dry THF (15 mL) at −78° C. under nitrogen atmosphere. After a further 0.5 h, a solution of C₂Cl₆ (2.86 g, 12.1 mmol) in THF (5 mL) was treated. The resulting reaction mixture was stirred at −78° C. for another 2 h and allowed to warm to rt over 14 h then quenched with saturated NaHCO₃ (10 mL). The aqueous phase was extracted with EtOAc (30 mL×3). The combined organic phases were dried over Na₂SO₄, filtered, and concentrated to give a brown oil that was subjected to flash chromatography on silica gel (5%, EtOAc/hexane), Collection of the appropriated fractions provided 2-chloro-5-(4-(difluoromethoxy)phenyl)oxazole as a white solid (1.78 g, 90% yield). ¹H NMR (400 MHz, CDCl₃) δ 7.58 (d, J=8.0 Hz, 2H), 7.25 (s, 1H), 7.17 (d, J=8.0 Hz, 2H), 6.55 (t, J=72 Hz, 1H, split by F). MS m/z=246.0 [M+H]⁺.

NH₄OH (28˜30% NH₃ basis aqueous solution, 10 mL) was added to a solution of 2-chloro-5-(4-(difluoromethoxy)phenyl)oxazole (1.2 g, 4.89 mmol) in THF (1.5 mL) at rt. The resulting reaction mixture was irradiated under microwave for 1 h at 90° C. The solid was filtered and washed with DCM (5 mL×2), providing 5-(4-(difluoromethoxy)phenyl)oxazol-2-amine as a white solid (1.05 g, 95% yield). ¹H NMR (400 MHz, DMSO-d₆) δ 7.50 (d, J=8.0 Hz, 2H), 7.20 (d, J=8.0 Hz, 2H), 7.20 (t, J=72 Hz, 1H, split by F), 6.86 (s, 2H). MS m/z=227.1 [M+H]⁺.

A degassed solution of 5-(4-(difluoromethoxy)phenyl)oxazol-2-amine (200 mg, 0.88 mmol), 5-bromopicolinonitrile (178 mg, 0.97 mmol), Cs₂CO₃ (430 mg, 1.32 mmol), Pd₂(dba)₃ (40 mg, 0.044 mmol), and Xantphos (51 mg, 0.088 mmol) in 1,4-dioxane (10 mL) was stirred for 5 h at 110° C. The volatile solvents were removed in vacuo. The residue was partitioned between EtOAc and H₂O. The aqueous phase was extracted with EtOAc (20 mL×3). The combined organic phases were dried over MgSO₄, filtered, and concentrated to give a yellow residue that was subjected to flash chromatography on silica gel (5%-50%, EtOAc/DCM). Collection of the appropriate fractions provided 5-((5-(4-(difluoromethoxy)phenyl) oxazol-2-yl)amino)picolinonitrile as a white solid (0.26 g, 89% yield). ¹H NMR (400 MHz, DMSO-d₆) δ 11.29 (s, 1H), 8.83 (d, J=2.5 Hz, 1H), 8.32 (dd, J=8.6, 2.5 Hz, 1H), 7.97 (d, J=8.6 Hz, 1H), 7.66 (d, J=8.8 Hz, 1H), 7.27 (d, J=8.8 Hz, 1H), 7.20 (t, J=74 Hz, 1H, split by F). MS m/z=329.1 [M+H]⁺.

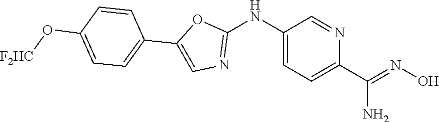 [SMILES: N/C(=N\O)c1ccc(Nc2ncc(-c3ccc(OC(F)F)cc3)o2)cn1]

Et₃N (0.81 mL, 5.85 mmol) and NH₂OH·HCl (406 mg, 5.85 mmol) was added to a suspension of 5-((5-(4-(difluoromethoxy)phenyl) oxazol-2-yl)amino)picolinonitrile (240 mg, 0.73 mmol) in EtOH (10 mL) at rt. The reaction mixture was stirred at reflux overnight. The volatile solvents were removed and the crude material was purified using preparative HPLC to provide 5-((5-(4-(difluoromethoxy)phenyl)oxazol-2-yl)amino)-N′-hydroxy picolinimidamide as a light yellow solid (0.022 g, 8.3% yield). ¹H NMR (400 MHz, DMSO-d₆) δ 11.15 (s, 1H), 10.79 (brs, 1H), 8.87 (d, J=2.5 Hz, 1H), 8.30 (dd, J=8.8, 2.5 Hz, 1H), 8.01 (d, J=8.8 Hz, 1H), 7.67 (d, J=8.8 Hz, 1H), 7.28 (d, J=8.8 Hz, 1H), 7.28 (t, J=74 Hz, 1H, split by F). ¹³C NMR (101 MHz, DMSO-d₆) δ 150.0, 143.8, 138.8, 138.0, 124.8, 124.6, 123.0, 122.5, 119.5, 116.3. LCMS R_{f}(min)=2.54, MS m/z=362.1 [M+H]⁺. HRMS (ESI) calcd for C₁₆H₁₄F₂N₅O₃⁺ [M+H]⁺ 362.1065, found 362.1045.

### 52. 5-((5-(4-(Difluoromethyl)phenyl)oxazol-2-yl)amino)-N′-hydroxypicolinimidamide (Scheme 49)

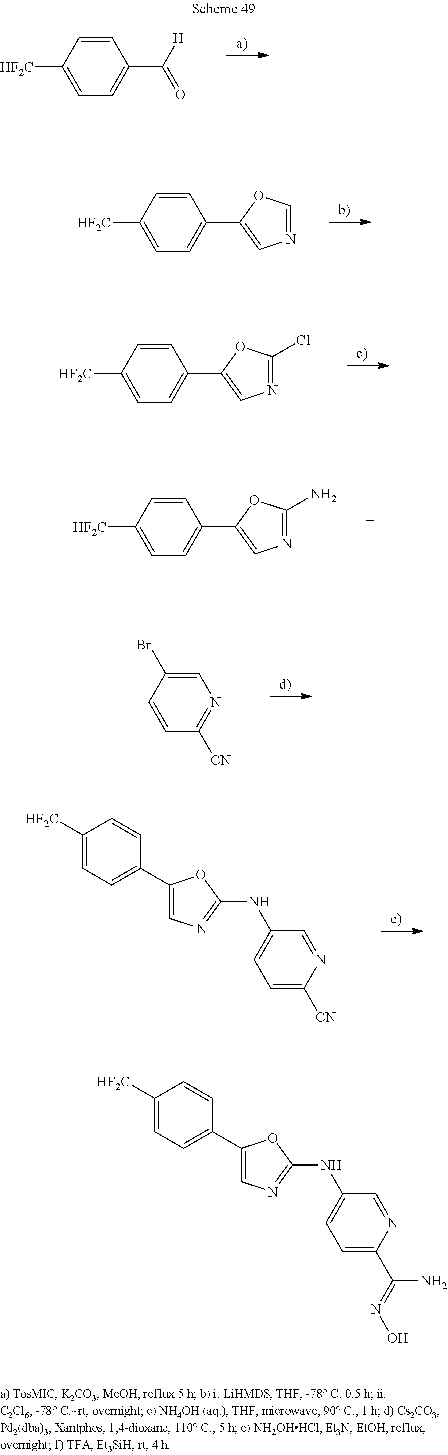

4-(Difluoromethyl)benzaldehyde (1.38 g, 8.84 mmol) and K₂CO₃ (1.47 g, 10.6 mmol) were added to a solution of TosMIC (1.90 g, 9.72 mmol) in MeOH (20 mL) at rt. The resulting mixture was then refluxed for 5 h under nitrogen before being concentrated under reduced pressure. The residue was partitioned between Et₂O and H₂O. The aqueous phase was extracted with Et₂O (30 mL×2), and the combined organic phases were dried over MgSO₄, filtered and concentrated to give a light yellow oil that was subjected to flash chromatography on silica gel (3%-20%, EtOAc/hexane). Collection of the appropriate fractions provided 5-(4-(difluoromethyl)phenyl)oxazole as a white solid. (1.40 g, 81% yield). ¹H NMR (400 MHz, CDCl₃) δ) δ 7.94 (s, 1H), 7.72 (d, J=8.2 Hz, 2H), 7.55 (d, J=8.2 Hz, 2H), 7.42 (s, 1H), 6.66 (t, J=56 Hz, 1H, split by F). MS m/z=196.0 [M+H]⁺.

LiHMDS (1.0 M in THF, 4.22 mL, 4.22 mmol) was added to a solution of 5-(4-(difluoromethyl)phenyl)oxazole (0.75 g, 3.84 mmol) in dry THF (10 mL) at −78° C. under nitrogen atmosphere. After a further 0.5 h, a solution of C₂Cl₆ (1.36 g, 5.76 mmol) in THF (5 mL) was treated. The resulting reaction mixture was stirred at −78° C. for another 2 h and allowed to warm to rt over 14 h. The reaction was quenched with saturated NaHCO₃ (10 mL). The aqueous phase was extracted with EtOAc (30 mL×2). The combined organic phases were dried over Na₂SO₄, filtered, and concentrated to give a brown oil that was subjected to flash chromatography on silica gel (2%-10%, EtOAc/hexane). Collection of the appropriate fractions provided 2-chloro-5-(4-(difluoromethyl)phenyl)oxazole as a white solid (0.73 g, 83% yield). ¹H NMR (400 MHz, CDCl₃) δ 7.77 (d, J=8.4 Hz, 2H), 7.56 (d, J=8.4 Hz, 2H), 7.35 (s, 1H), 6.66 (t, J=56 Hz, 1H, split by F). MS m/z=230.0 [M+H]⁺.

NH₄OH (28-30% NH₃ basis aqueous solution, 10 mL) was added to a solution of 2-chloro-5-(4-(difluoromethyl)phenyl)oxazole) (0.75 g, 3.27 mmol) in THF (1.5 mL) at rt. The resulting reaction mixture was radiated under microwave for 1 h at 90° C. The solid was filtered and washed with DCM (5 mL×2), providing 5-(4-(difluoromethyl)phenyl)oxazol-2-amine as a yellow solid (0.66 g, 96% yield). ¹H NMR (400 MHz, DMSO) δ 7.52-7.61 (m, 4H), 7.33 (s, 1H), 7.14-6.86 (m, 3H). MS m/z=211.0 [M+H]⁺.

A degassed solution of 5-(4-(difluoromethyl)phenyl)oxazol-2-amine (152 mg, 0.72 mmol), 5-bromopicolinonitrile (199 mg, 1.08 mmol), Cs₂CO₃ (469 mg, 1.44 mmol), Pd₂(dba)₃ (53 mg, 0.058 mmol), and Xantphos (42 mg, 0.072 mmol) in 1,4-dioxane (10 mL) was stirred for 5 h at 110° C. The volatile solvents were removed in vacuo. The residue was partitioned between EtOAc and H₂O. The aqueous phase was extracted with EtOAc (20 mL×3). The combined organic phases were dried over MgSO₄, filtered, and concentrated to give a grey residue that was subjected to flash chromatography with EtOAc/DCM (5% to 50%). Collection of the appropriate fractions provided 5-((5-(4-(difluoromethyl)phenyl)oxazol-2-yl)amino)picolinonitrile as a white solid (0.20 g, 90% yield). ¹H NMR (400 MHz, DMSO-d₆) δ 11.36 (s, 1H), 8.85 (d, J=2.5 Hz, 1H), 8.32 (dd, J=8.6, 2.5 Hz, 1H), 7.98 (d, J=8.6 Hz, 1H), 7.75 (d, J=8.2 Hz, 1H), 7.71 (s, 1H), 7.65 (d, J=8.2 Hz, 1H), 7.05 (t, J=56 Hz, 1H, split by F). MS m/z=313.1 [M+H]⁺.

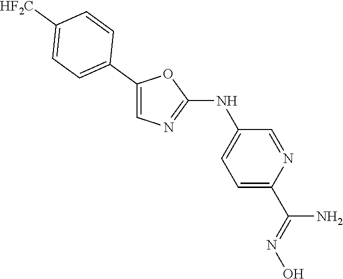

Et₃N (0.57 mL, 4.10 mmol) and NH₂OH·HCl (284 mg, 4.10 mmol) was added to a suspension of 5-((5-(4-(difluoromethyl)phenyl)oxazol-2-yl)amino)picolinonitrile (160 mg, 0.51 mmol) in EtOH (8 mL) at rt. The reaction mixture was stirred at reflux overnight. The volatile solvents were removed and the crude material was purified using preparative HPLC to provide 5-((5-(4-(difluoromethyl)phenyl)oxazol-2-yl)amino)-N′-hydroxypicolinimidamide as a brown solid (0.016 g, 9.1% yield). ¹H NMR (400 MHz, DMSO-d₆) δ 11.19 (s, 1H), 10.70 (brs, 1H), 8.87 (d, J=2.3 Hz, 1H), 8.29 (dd, J=8.7, 2.3 Hz, 1H), 8.00 (d, J=8.7 Hz, 1H), 7.75 (d, J=8.2 Hz, 2H), 7.70 (s, 1H), 7.65 (d, J=8.2 Hz, 2H), 7.05 (t, J=56 Hz, 1H, split by F). ¹³C NMR (101 MHz, DMSO-d₆) δ 155.8, 143.9, 138.0, 132.9, 132.7, 132.5, 130.0, 126.6(4), 126.5(8), 126.5(2), 124.2, 123.3, 123.0, 122.4, 117.1, 114.8, 112.4. LCMS R_{f}(min)=2.65, MS m/z=346.1 [M+H]⁺. HRMS (ESI) calcd for C₁₆H₁₄F₂N₅O₂⁺ [M+H]⁺ 346.1116, found 346.1107.

### 53. N′-Hydroxy-6-((5-(4-(trifluoromethyl)phenyl) oxazol-2-yl)amino)pyridazine-3-carboximidamide (Scheme 50)

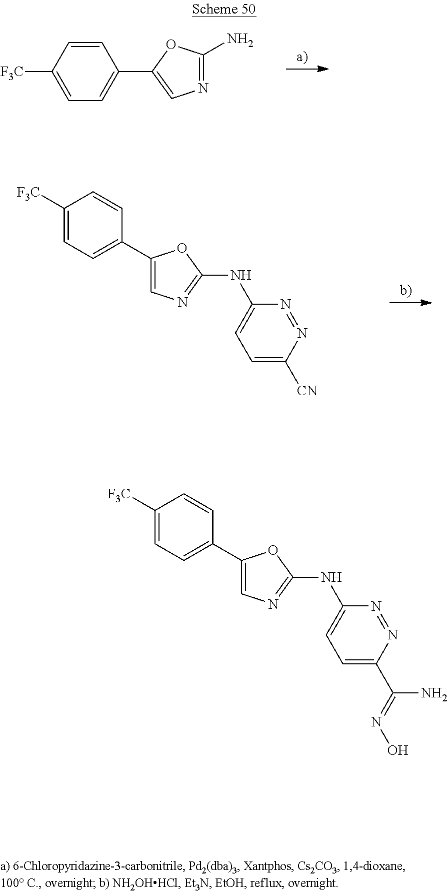 [SMILES: Nc1ncc(-c2ccc(C(F)(F)F)cc2)o1 ; N#Cc1ccc(Nc2ncc(-c3ccc(C(F)(F)F)cc3)o2)nn1 ; N/C(=N\O)c1ccc(Nc2ncc(-c3ccc(C(F)(F)F)cc3)o2)nn1]

A re-sealable Schlenk tube was charged with Pd₂(dba)₃ (0.05 equiv) Xantphos (0.1 equiv), 6-chloropyridazine-3-carbonitrile (1.1 equiv), Cs₂CO₃ (1.3 equiv) and 5-(4-(trifluoromethyl)phenyl)oxazol-2-amine (Intermediate D) (0.2 g, 0.88 mmoL) in 1,4-dioxane (8 mL). The mixture was degassed and carefully subjected to three cycles of evacuation-backfilling with N₂ and heated at 100° C. overnight. After completion, the mixture was cooled and solvent evaporated to obtain crude, which was stirred with 5% aqueous solution of potassium xanthogenate (10 mL) for 5 min, filtered, washed with water (2×10 mL), MeOH (3×5 mL) and Et₂O (3×5 mL) to obtain pure product as yellow solid (210 mg, 72.3%). ¹H NMR (401 MHz, DMSO-d₆) δ 8.44 (d, J=7.9 Hz, 1H), 8.24 (d, J=9.3 Hz, 1H), 7.82 (s, 5H). LCMS R_{f}(min)=3.604. MS m/z=332.1 [M+H]⁺.

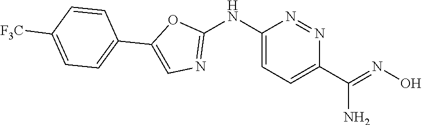 [SMILES: N/C(=N\O)c1ccc(Nc2ncc(-c3ccc(C(F)(F)F)cc3)o2)nn1]

6-((5-(4-(Trifluoromethyl)phenyl)oxazol-2-yl)amino)pyridazine-3-carbonitrile (0.15 g) was suspended in EtOH (15 mL) followed by the addition of NH₂OH·HCl (8.0 equiv) and Et₃N (8 equiv). The resulting mixture was refluxed overnight. After completion, mixture was cooled and solid filtered, washed with water (2×10 mL), MeOH (3×5 mL) and Et₂O (2×5 mL) to obtain pure product as beige solid (90 mg, 54.6%). ¹H NMR (401 MHz, DMSO-d₆) δ 11.98 (s, 1H), 10.16 (s, 1H), 8.25 (s, 1H), 8.02 (s, 1H), 7.82 (s, 5H), 5.97 (s, 2H). LCMS R_{f}(min)=3.463. HRMS (ESI) calcd for C₁₅H₁₂F₃N₆O₂⁺ [M+H]⁺ 365.0968, found 365.0975.

### 54. N′-Hydroxy-5-((5-(4-(trifluoromethyl)phenyl)oxazol-2-yl)amino)pyrazine-2-carboximidamide (Scheme 51)

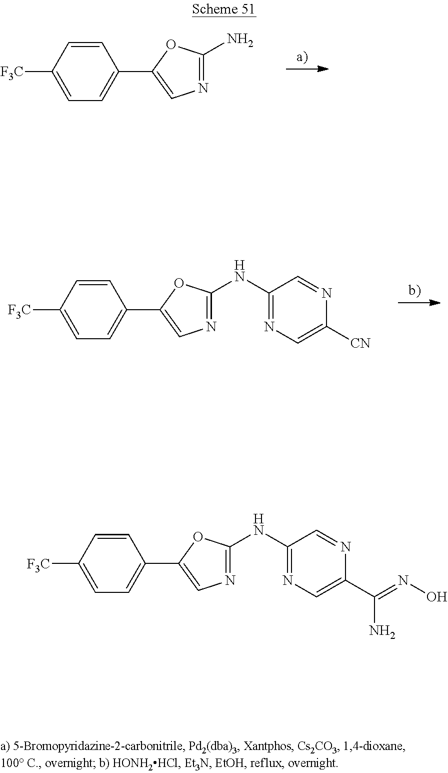 [SMILES: Nc1ncc(-c2ccc(C(F)(F)F)cc2)o1 ; N#Cc1cnc(Nc2ncc(-c3ccc(C(F)(F)F)cc3)o2)cn1 ; N/C(=N\O)c1cnc(Nc2ncc(-c3ccc(C(F)(F)F)cc3)o2)cn1]

A re-sealable Schlenk tube was charged with Pd₂(dba)₃ (0.05 equiv), Xantphos (0.1 equiv), 5-bromopyrazine-2-carbonitrile (1.1 equiv), Cs₂CO₃ (1.3 equiv) and 5-(4-(trifluoromethyl)phenyl)oxazol-2-amine (Intermediate D) (0.2 g, 0.877 mmoL) in 1,4-dioxane. The mixture was carefully subjected to three cycles of evacuation-backfilling with N₂ and heated at 100° C. overnight. After completion, the mixture was cooled and solvent evaporated to obtain crude, which was stirred with 5% aqueous solution of potassium xanthogenate (10 mL) for 5 min, filtered, washed with water (3×10 mL), MeOH (3×5 mL) and Et₂O (2×5 mL) to obtain pure product as yellow solid (200 mg, 69%). ¹H NMR (401 MHz, DMSO-d₆) δ 12.24 (s, 1H), 9.25 (s, 1H), 8.87 (s, 1H), 7.86 (s, 1H), 7.83 (m, 4H). LCMS R_{f}(min)=3.551, MS m/z=332.0 [M+H]⁺.

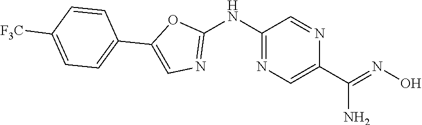 [SMILES: N/C(=N\O)c1cnc(Nc2ncc(-c3ccc(C(F)(F)F)cc3)o2)cn1]

5-((5-(4-(Trifluoromethyl)phenyl)oxazol-2-yl)amino)pyrazine-2-carbonitrile (0.15 g) was suspended in EtOH (15 mL) followed by the addition of NH₂OH·HCl (8.0 equiv) and Et₃N (8.0 equiv). The resulting mixture was refluxed overnight. After completion, the mixture was cooled and solid filtered, washed with water (2×10 mL), MeOH (3×5 mL), 1,4-dioxane:Et₂O (1:1, 3×5 mL), and Et₂O (2×5 mL) to provide N′-hydroxy-5-((5-(4-(trifluoromethyl)phenyl)oxazol-2-yl)amino)pyrazine-2-carboximidamide as beige solid (70 mg, 42%). ¹H NMR (401 MHz, DMSO-d₆) δ 11.59 (s, 1H), 9.94 (s, 1H), 9.23 (s, 1H), 8.75 (s, 1H), 7.82 (br s, 5H), 5.85 (s, 2H). LCMS R_{f} (min)=3.697. HRMS (ESI) calcd for C₁₅H₁₂F₃N₆O₂⁺ [M+H]⁺ 365.0968, found 365.0984.

### 55. N′-Hydroxy-5-((5-(4-(trifluoromethyl)phenyl)oxazol-2-yl)amino)pyrimidine-2-carboximidamide (Scheme 52)

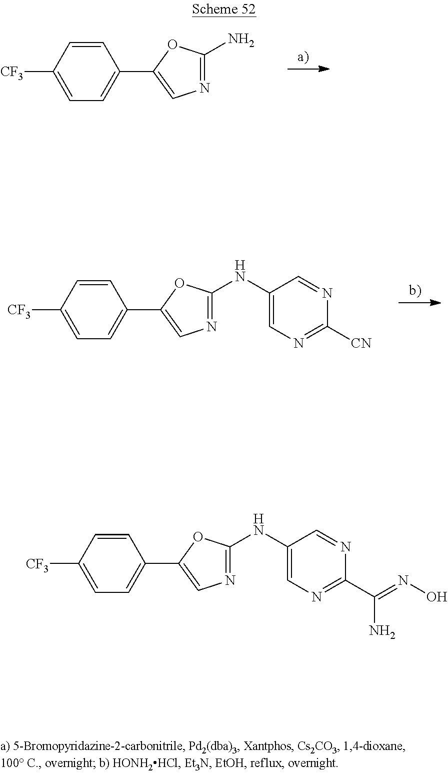 [SMILES: Nc1ncc(-c2ccc(C(F)(F)F)cc2)o1 ; N#Cc1ncc(Nc2ncc(-c3ccc(C(F)(F)F)cc3)o2)cn1 ; N/C(=N\O)c1ncc(Nc2ncc(-c3ccc(C(F)(F)F)cc3)o2)cn1]

A re-sealable Schlenk tube was charged with Pd₂(dba)₃ (0.05 equiv), Xantphos (0.1 equiv), 5-bromopyrimidine-2-carbonitrile (1.1 equiv), Cs₂CO₃ (1.3 equiv) and 5-(4-(trifluoromethyl)phenyl)oxazol-2-amine (Intermediate D) (0.2 g, 0.877 mmoL) in 1,4-dioxane (8 mL). The mixture was degassed and carefully subjected to three cycles of evacuation-backfilling with N₂ and heated at 100° C. overnight. After completion, the mixture was cooled and solvent evaporated to obtain crude, which was stirred with 5% aqueous solution of potassium xanthogenate (10 mL) for 5 min, filtered, washed with water (3×10 mL), MeOH (3×5 mL) and Et₂O (2×5 mL) to provide 5-((5-(4-(trifluoromethyl)phenyl)oxazol-2-yl)amino)pyrimidine-2-carbonitrile as beige solid (200 mg, 69%). The compound was used as such without any further purification.

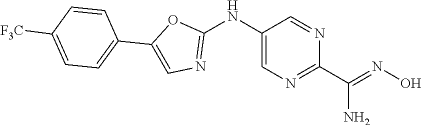 [SMILES: N/C(=N\O)c1ncc(Nc2ncc(-c3ccc(C(F)(F)F)cc3)o2)cn1]

5-((5-(4-(Trifluoromethyl)phenyl)oxazol-2-yl)amino)pyrimidine-2-carbonitrile (0.11 g) was suspended in EtOH (10 mL) followed by the addition of NH₂OH·HCl (8.0 equiv) and Et₃N (8.0 equiv). The resulting mixture was refluxed overnight. After completion, the mixture was cooled and solid filtered, washed with water (2×10 mL), MeOH (2×5 mL), 1,4-dioxane:Et₂O (1:1, 3×5 mL) and Et₂O (2×5 mL) to provide N′-hydroxy-5-((5-(4-(trifluoromethyl)phenyl)oxazol-2-yl)amino)pyrimidine-2-carboximidamide as beige solid (50 mg, 41.3%). ¹H NMR (401 MHz, DMSO-d₆) δ 11.28 (s, 1H), 9.79 (s, 1H), 8.80 (s, 2H), 7.82 (m, 5H), 6.01 (s, 2H). LCMS R_{f}(min)=3.597. HRMS (ESI) calcd for C₁₅H₁₂F₃N₆O₂⁺ [M+H]⁺ 365.0968, found 365.0982.

### 56. 3-Fluoro-N′-hydroxy-5-((5-(5-(trifluoromethyl)pyridin-2-yl)oxazol-2-yl)amino)picolinimidamide (Scheme 53)

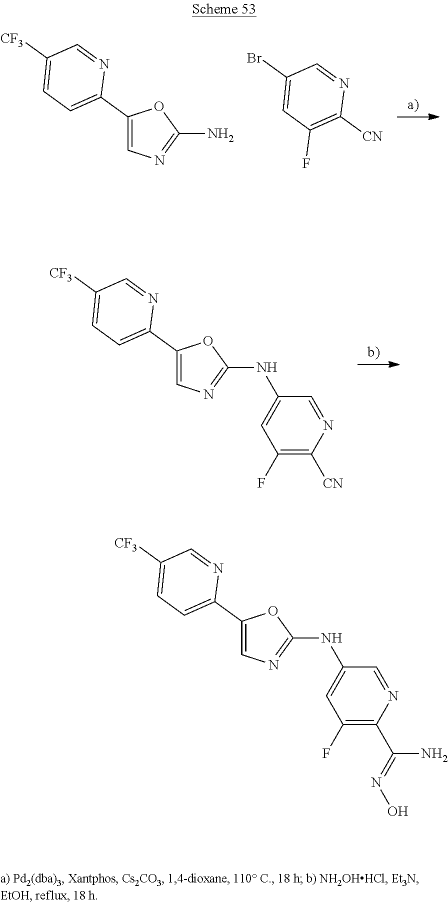 [SMILES: Nc1ncc(-c2ccc(C(F)(F)F)cn2)o1 ; N#Cc1ncc(Br)cc1F ; N#Cc1ncc(Nc2ncc(-c3ccc(C(F)(F)F)cn3)o2)cc1F ; N/C(=N\O)c1ncc(Nc2ncc(-c3ccc(C(F)(F)F)cn3)o2)cc1F]

Intermediate K (5-(5-(trifluoromethyl)pyridin-2-yl)oxazol-2-amine) (0.2 g, 0.872 mmol) was reacted with 5-bromo-3-fluoropicolinonitrile (0.175 g, 0.872 mmol) following General Procedure 4 Method 1. The crude product was purified on silica gel using mixtures of DCM and EtOAc (0 to 30%) as eluents to provide the desired 3-fluoro-5-((5-(5-(trifluoromethyl)pyridin-2-yl)oxazol-2-yl)amino)picolinonitrile in 73.16% yield (0.223 g). ¹H NMR (401 MHz, DMSO-d₆) δ 11.99 (br s, 1H), 8.95 (s, 1H), 8.60 (s, 1H), 8.35 (d, J 11.5 Hz, 1H), 8.29 (d, J=8.3 Hz, 1H), 7.97 (s, 1H), 7.83 (d, J=8.3 Hz, 1H). LCMS R_{f}(min)=3.351, MS m/z=350.1 [M+H]⁺.

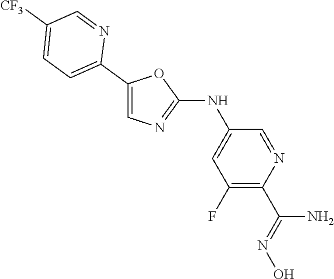 [SMILES: N/C(=N\O)c1ncc(Nc2ncc(-c3ccc(C(F)(F)F)cn3)o2)cc1F]

3-Fluoro-5-((5-(5-(trifluoromethyl)pyridin-2-yl)oxazol-2-yl)amino)picolinonitrile (0.21 g, 0.6 mmol) was subjected to amidoxime formation in EtOH as per General Procedure 1 Method 2 to furnish 3-fluoro-N′-hydroxy-5-((5-(5-NH₂ (trifluoromethyl)pyridin-2-yl)oxazol-2-yl)amino)picolinimidamide (91.8%, 211 mg) after filtering with water, then washing with water, EtOH, and Et₂O. ¹H NMR (401 MHz, DMSO-d₆) δ 9.95 (s, 1H), 8.94 (s, 1H), 8.58 (s, 1H), 8.27 (d, J=6.7 Hz, 1H), 8.14 (dd, J=13.1, 1.8 Hz, 1H), 7.92 (s, 1H), 7.80 (d, J=8.4 Hz, 1H), 5.76 (s, 2H). LCMS R_{f}(min)=3.027, MS m/z=383.1 [M+H]⁺. HRMS (ESI) calcd for C₁₅H₁₁F₄N₆O₂⁺ [M+H]⁺ 383.0874, found 383.0871.

### 57. N′-Hydroxy-5-((5-(5-(trifluoromethyl)pyridin-2-yl)oxazol-2-yl)amino)pyrazine-2-carboximidamide (Scheme 54)

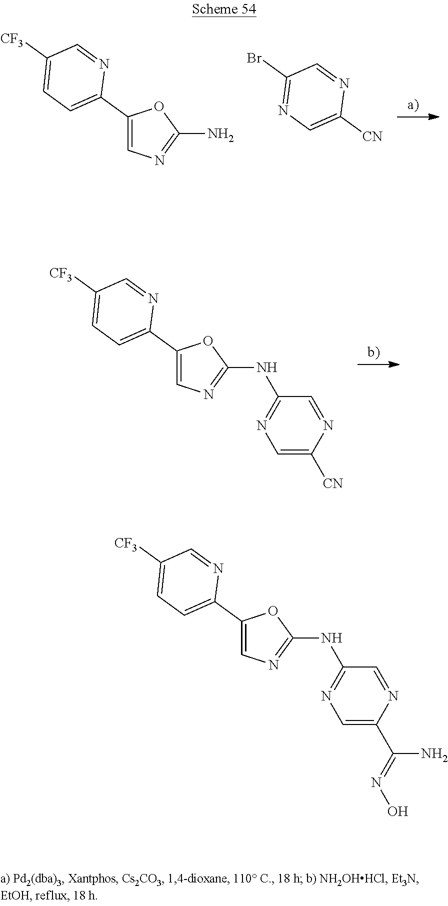 [SMILES: Nc1ncc(-c2ccc(C(F)(F)F)cn2)o1 ; N#Cc1cnc(Br)cn1 ; N#Cc1cnc(Nc2ncc(-c3ccc(C(F)(F)F)cn3)o2)cn1 ; N/C(=N\O)c1cnc(Nc2ncc(-c3ccc(C(F)(F)F)cn3)o2)cn1]

Intermediate K (5-(5-(trifluoromethyl)pyridin-2-yl)oxazol-2-amine) (0.156 g, 0.68 mmol) was reacted with 5-bromopyrazine-2-carbonitrile (0.125 g, 0.68 mmol) following General Procedure 4 Method 1. The crude product was purified on silica gel using mixtures of DCM and EtOAc (0 to 70%) as eluents to provide the desired 5-((5-(5-(trifluoromethyl)pyridin-2-yl)oxazol-2-yl)amino)pyrazine-2-carbonitrile in 32.34% yield (0.073 g). ¹H NMR (401 MHz, DMSO-d₆) δ 12.36 (br s, 1H), 9.29 (s, 1H), 8.94 (s, 1H), 8.88 (d, J=0.9 Hz, 1H), 8.28 (dd, J=8.4, 1.9 Hz, 1H), 7.95 (s, 1H), 7.81 (d, J=8.4 Hz, 1H). LCMS R_{f}(min)=2.961, MS m/z=333.0 [M+H]⁺.

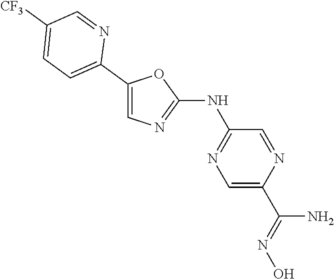 [SMILES: N/C(=N\O)c1cnc(Nc2ncc(-c3ccc(C(F)(F)F)cn3)o2)cn1]

5-((5-(5-(Trifluoromethyl)pyridin-2-yl)oxazol-2-yl)amino)pyrazine-2-carbonitrile (0.06 g, 0.180 mmol) was subjected to amidoxime formation in EtOH as per General Procedure 1 Method 2 to furnish N′-hydroxy-5-((5-(5-(trifluoromethyl)pyridin-2-yl)oxazol-2-yl)amino)pyrazine-2-carboximidamide (77.3%, 51 mg) after filtering with water, then washing with water, EtOH, and Et₂O. ¹H NMR (401 MHz, DMSO-d₆) δ 11.75 (s, 1H), 9.96 (s, 1H), 9.26 (s, 1H), 8.95 (s, 1H), 8.76 (s, 1H), 8.29 (d, J=7.4 Hz, 1H), 7.95 (s, 1H), 7.81 (d, J=8.2 Hz, 1H), 5.86 (s, 2H). LCMS R_{f}(min)=2.936, MS m/z=366.1 [M+H]⁺. HRMS (ESI) calcd for C₁₄H₁₁F₃N₇O₂⁺ [M+H]⁺ 366.0921, found 366.0932.

### 58. N′-Hydroxy-5-((5-(5-(trifluoromethyl)pyridin-2-yl)oxazol-2-yl)amino)pyrimidine-2-carboximidamide (Scheme 55)

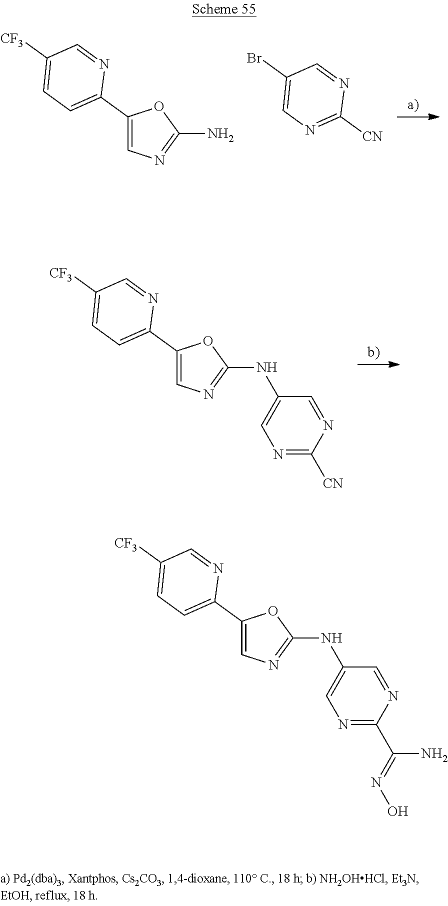 [SMILES: Nc1ncc(-c2ccc(C(F)(F)F)cn2)o1 ; N#Cc1ncc(Br)cn1 ; N#Cc1ncc(Nc2ncc(-c3ccc(C(F)(F)F)cn3)o2)cn1 ; N/C(=N\O)c1ncc(Nc2ncc(-c3ccc(C(F)(F)F)cn3)o2)cn1]

Intermediate K (5-(5-(trifluoromethyl)pyridin-2-yl)oxazol-2-amine) (0.156 g, 0.68 mmol) was reacted with 5-bromopyrimidine-2-carbonitrile (0.125 g, 0.68 mmol) following General Procedure 4 Method 1. The crude product was purified on silica gel using mixtures of DCM and EtOAc (0 to 30%) as eluents to provide the desired 5-((5-(5-(trifluoromethyl)pyridin-2-yl)oxazol-2-yl)amino)pyrimidine-2-carbonitrile in 25.25% yield (0.057 g). ¹H NMR (401 MHz, DMSO-d₆) δ 9.17 (s, 2H), 8.96 (d, J=0.9 Hz, 1H), 8.28 (dd, J=8.5, 2.1 Hz, 1H), 7.97 (s, 1H), 7.84 (d, J=8.4 Hz, 1H). LCMS R_{f}(min)=3.072, MS m/z=333.1 [M+H]⁺.

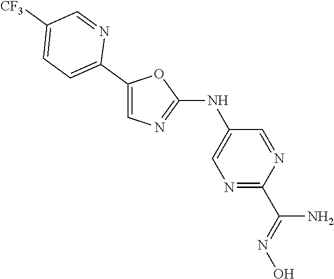 [SMILES: N/C(=N\O)c1ncc(Nc2ncc(-c3ccc(C(F)(F)F)cn3)o2)cn1]

5-((5-(5-(Trifluoromethyl)pyridin-2-yl)oxazol-2-yl)amino)pyrimidine-2-carbonitrile (0.051 g, 0.153 mmol) was subjected to amidoxime formation in EtOH as per General Procedure 1 Method 2 to furnish N′-hydroxy-5-((5-(5-(trifluoromethyl)pyridin-2-yl)oxazol-2-yl)amino)pyrimidine-2-carboximidamide (85.1%, 47.7 mg) after purification via preparative HPLC. ¹H NMR (401 MHz, DMSO-d₆) δ 11.70 (br s, 1H), 11.04-10.92 (br s, 1H), 9.20 (s, 2H), 8.97-8.94 (m, 1H), 8.29 (dd, J=8.7, 2.1 Hz, 1H), 8.24-8.18 (br s, 1H), 7.97 (s, 1H), 7.83 (d, J=8.4 Hz, 1H). LCMS R_{f}(min)=2.779, MS m/z=366.1 [M+H]⁺. HRMS (ESI) calcd for C₁₄H₁₁F₃N₇O₂⁺ [M+H]⁺ 366.0921, found 366.0921.

### 59. N′-Hydroxy-5-((5-(6-(trifluoromethyl)pyridin-3-yl)oxazol-2-yl)amino)picolinimidamide (Scheme 56)

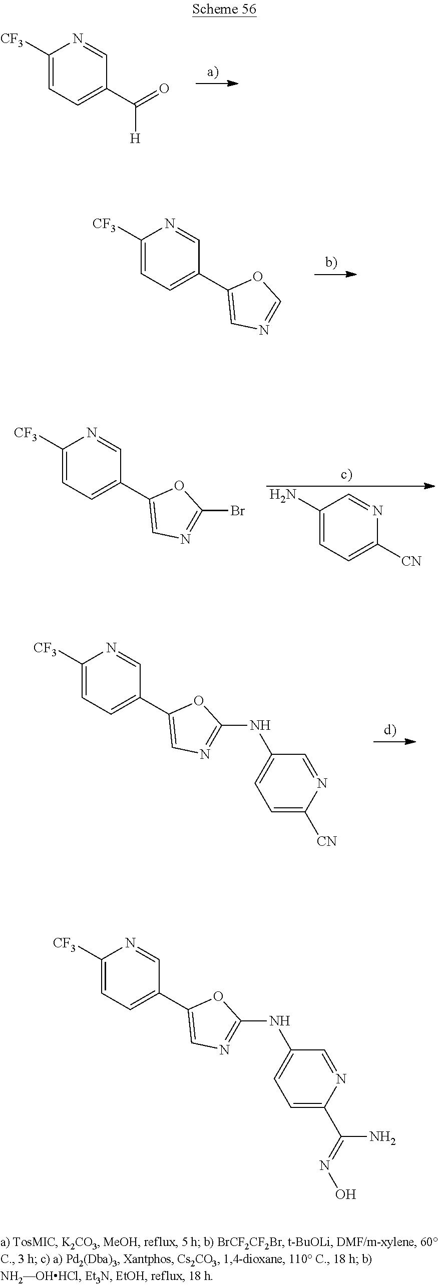 [SMILES: O=Cc1ccc(C(F)(F)F)nc1 ; FC(F)(F)c1ccc(-c2cnco2)cn1 ; FC(F)(F)c1ccc(-c2cnc(Br)o2)cn1 ; N#Cc1ccc(N)cn1 ; N#Cc1ccc(Nc2ncc(-c3ccc(C(F)(F)F)nc3)o2)cn1 ; N/C(=N\O)c1ccc(Nc2ncc(-c3ccc(C(F)(F)F)nc3)o2)cn1]

6-(Trifluoromethyl)nicotinaldehyde (1.2 g, 6.85 mmol) was reacted with TosMIC (1.54 g, 7.88 mmol) and K₂CO₃ (1.14 g, 8.22 mmol) in MeOH (20 mL) as per General Procedure 14 to provide 5-(6-(trifluoromethyl)pyridin-3-yl)oxazole in 89% yield (1.31 g) as a white solid. ¹H NMR (401 MHz, CDCl₃) δ 9.01 (d, J=1.9 Hz, 1H), 8.10 (dd, J=8.2, 1.6 Hz, 1H), 8.03 (s, 1H), 7.72 (m, 1H), 7.57 (s, 1H). LCMS R_{f}(min)=2.622, MS m/z=215.1 [M+H]⁺.

5-(6-(Trifluoromethyl)pyridin-3-yl)oxazole (1.04 g, 4.86 mmol) was reacted with BrCF₂CF₂Br (0.93 mL, 7.77 mmol) and t-BuOLi (0.622 g, 7.77 mmol) in DMF/m-xylene (5/5 mL) at 60° C. for 3 h as per General Procedure 15 Method 2 to provide 2-bromo-5-(6-(trifluoromethyl)pyridin-3-yl)oxazole (0.678 g, 48%) as a yellow solid. ¹H NMR (401 MHz, CDCl₃) δ 8.95 (s, 1H), 8.06 (d, J=7.6 Hz, 1H), 7.75 (d, J=8.0 Hz, 1H), 7.52 (s, 1H). LCMS R_{f}(min)=3.338, MS m/z=295.0 [M+H]⁺.

2-Bromo-5-(6-(trifluoromethyl)pyridin-3-yl)oxazol (0.12 g, 0.68 mmol) was reacted with 5-aminonicotinonitrile (0.405 g, 3.4 mmol) following General Procedure 4 Method 1. The crude product was purified preparative HPLC to provide 5-((5-(6-(trifluoromethyl)pyridin-3-yl)oxazol-2-yl)amino)picolinonitrile in 50.1% yield (0.113 g). ¹H NMR (401 MHz, DMSO-d₆) δ 11.67-11.30 (br s, 1H), 9.01 (s, 1H), 8.83 (d, J=2.4 Hz, 1H), 8.32 (dd, J=8.7, 2.6 Hz, 1H), 8.20 (m, 1H), 7.97 (m, 2H), 7.93 (s, 1H). LCMS R_{f}(min)=3.449, MS m/z=332.1 [M+H]⁺.

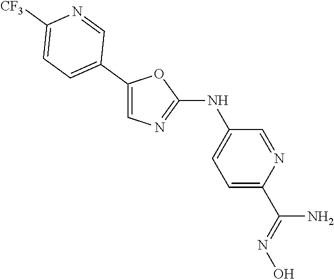 [SMILES: N/C(=N\O)c1ccc(Nc2ncc(-c3ccc(C(F)(F)F)nc3)o2)cn1]

5-((5-(6-(trifluoromethyl)pyridin-3-yl)oxazol-2-yl)amino)picolinonitrile (0.1 g, 0.301 mmol) was subjected to amidoxime formation as per General Procedure 1 Method 2 to furnish N′-hydroxy-5-((5-(6-(trifluoromethyl)pyridin-3-yl)oxazol-2-yl)amino)picolinimidamide (85.24%, 123 mg, treated as TFA salt) after purification via preparative HPLC. ¹H NMR (401 MHz, DMSO-d₆) δ 11.40 (s, 1H), 10.89 (br s, 1H), 9.02 (d, J=2.0 Hz, 1H), 8.89 (d, J=2.2 Hz, 1H), 8.32 (dd, J=8.8, 2.5 Hz, 1H), 8.21 (dd, J=8.2, 1.8 Hz, 1H), 8.03 (d, J=8.8 Hz, 1H), 7.98 (d, J=8.1 Hz, 1H), 7.93 (s, 1H). ¹³C NMR (101 MHz, DMSO) δ 158.79, 158.46, 157.06, 154.68, 144.84, 144.66, 144.50, 141.46, 138.88, 138.78, 131.66, 127.47, 127.42, 126.19, 123.90, 123.47, 123.21, 121.67, 120.76. LCMS R_{f}(min)=2.849, MS m/z=365.1 [M+H]⁺. HRMS (ESI) calcd for C₁₅H₁₂F₃N₆O₂⁺ [M+H]⁺ 365.0968, found 365.0973.

### 60. N′-Hydroxy-5-((4-(4-(trifluoromethyl)phenyl)oxazol-2-yl)amino)picolinimidamide (Scheme 57)

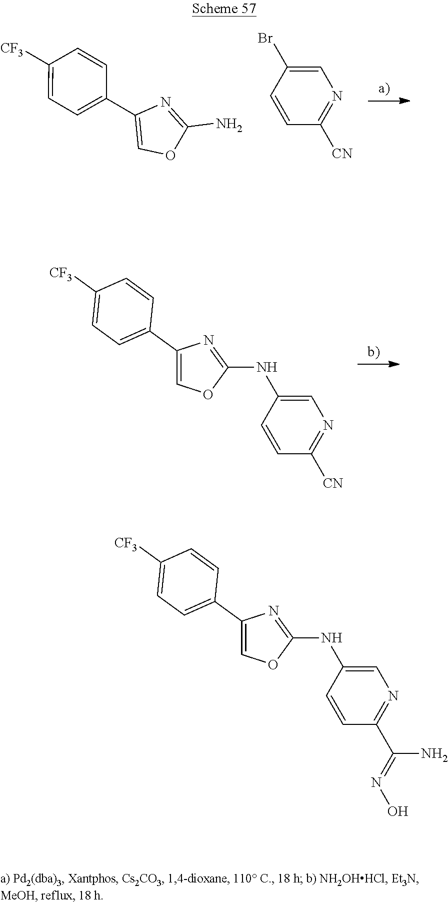 [SMILES: Nc1nc(-c2ccc(C(F)(F)F)cc2)co1 ; N#Cc1ccc(Br)cn1 ; N#Cc1ccc(Nc2nc(-c3ccc(C(F)(F)F)cc3)co2)cn1 ; N/C(=N\O)c1ccc(Nc2nc(-c3ccc(C(F)(F)F)cc3)co2)cn1]

Intermediate E (4-(4-(trifluoromethyl)phenyl)oxazol-2-amine) (0.935 g, 4.1 mmol) was reacted with 5-bromopicolinonitrile (0.75 g, 4.1 mmol) following General Procedure 4 Method 1. The crude product was purified on silica gel using mixtures of DCM and EtOAc (0 to 20%) as eluents to provide the desired 5-((4-(4-(trifluoromethyl)phenyl)oxazol-2-yl)amino)picolinonitrile in 13.2% yield (0.179 g). ¹H NMR (401 MHz, DMSO-d₆) δ 11.22 (s, 1H), 8.87 (d, J=2.3 Hz, 1H), 8.47 (s, 1H), 8.42 (dd, J=8.7, 2.6 Hz, 1H), 8.04-7.94 (m, 3H), 7.79 (d, J=8.3 Hz, 2H). LCMS R_{f}(min)=3.542, MS m/z=331.0 [M+H]⁺.

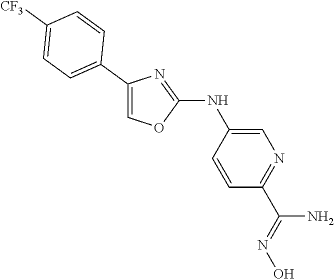 [SMILES: N/C(=N\O)c1ccc(Nc2nc(-c3ccc(C(F)(F)F)cc3)co2)cn1]

5-((4-(4-(Trifluoromethyl)phenyl)oxazol-2-yl)amino)picolinonitrile (0.153 g, 0.463 mmol) was subjected to amidoxime formation in MeOH as per General procedure 1 Method 2 to furnish N′-hydroxy-5-((4-(4-(trifluoromethyl)phenyl)oxazol-2-yl)amino)picolinimidamide (77.96%, corrected for 0.5 molar equivalent of dioxane, 147 mg) after isolation via filtering with water, and washing with water and Et₂O. ¹H NMR (401 MHz, DMSO-d₆) δ 10.73 (s, 1H), 9.81 (s, 1H), 8.91 (d, J=2.2 Hz, 1H), 8.42 (s, 1H), 8.17 (dd, J=8.8, 2.6 Hz, 1H), 8.01 (d, J=8.0 Hz, 2H), 7.87 (d, J=8.8 Hz, 1H), 7.80 (d, J=8.2 Hz, 2H), 5.91 (s, 2H). ¹³C NMR (101 MHz, DMSO-d₆) δ 156.86, 150.14, 143.13, 138.33, 137.14, 136.94, 135.66, 130.95, 128.43, 128.11, 126.18, 126.14, 126.10, 125.96, 124.20, 123.42, 120.32. LCMS R_{f}(min)=3.163, MS m/z=364.1 [M+H]⁺. HRMS (ESI) calcd for C₁₆H₁₃F₃N₅O₂⁺ [M+H]⁺ 364.1016, found 364.1019.

### 61. 6-((5-(5-(Trifluoromethyl)pyridin-2-yl)oxazol-2-yl)amino)pyridazine-3-carbonitrile (Scheme 58)

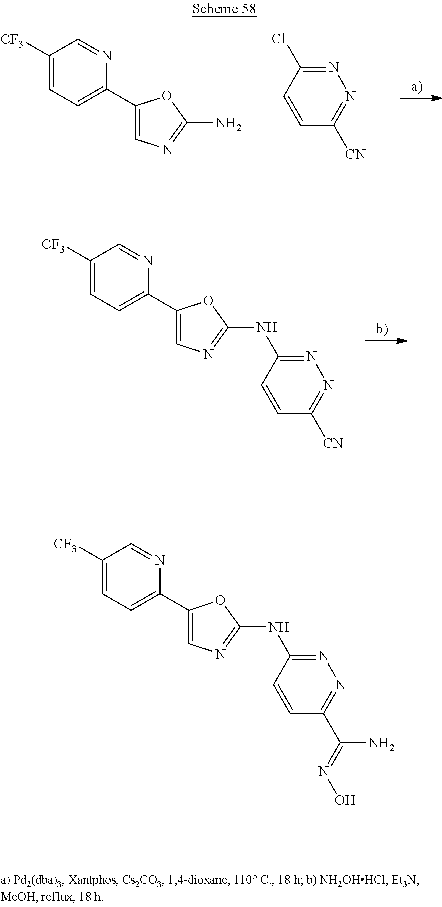 [SMILES: Nc1ncc(-c2ccc(C(F)(F)F)cn2)o1 ; N#Cc1ccc(Cl)nn1 ; N#Cc1ccc(Nc2ncc(-c3ccc(C(F)(F)F)cn3)o2)nn1 ; N/C(=N\O)c1ccc(Nc2ncc(-c3ccc(C(F)(F)F)cn3)o2)nn1]

Intermediate K (5-(5-(trifluoromethyl)pyridin-2-yl)oxazol-2-amine) (0.197 g, 0.86 mmol) was reacted with 6-chloropyridazine-3-carbonitrile (0.12 g, 0.86 mmol) following General Procedure 4 Method 1. Upon completion, the volatile solvents were removed in vacuo, and the solid residue, triturated with water, filtered and washed well with water. The resultant crude product was washed with Et₂O and DCM until all catalysts and ligands were removed, providing the desired 6-((5-(5-(trifluoromethyl)pyridin-2-yl)oxazol-2-yl)amino)pyridazine-3-carbonitrile in 79.4% yield (0.227 g). ¹H NMR (401 MHz, DMSO-d₆) δ 8.96 (s, 1H), 8.47 (m, 1H), 8.29 (m, 2H), 7.97 (s, 1H), 7.83 (d, J=8.4 Hz, 1H), 7.42 (m, 1H). LCMS R_{f}(min)=3.361, MS m/z=333.1 [M+H]⁺.

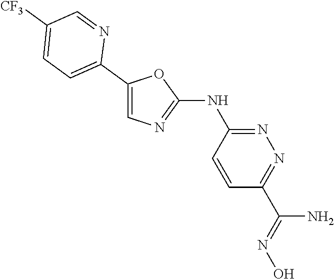 [SMILES: N/C(=N\O)c1ccc(Nc2ncc(-c3ccc(C(F)(F)F)cn3)o2)nn1]

6-((5-(5-(Trifluoromethyl)pyridin-2-yl)oxazol-2-yl)amino)pyridazine-3-carbonitrile (0.207 g, 0.623 mmol) was subjected to amidoxime formation in MeOH as per General Procedure 1 Method 2 to furnish N′-hydroxy-6-((5-(5-(trifluoromethyl)pyridin-2-yl)oxazol-2-yl)amino)pyridazine-3-carboximidamide (87%, 198 mg) after isolation via filtering with water, and washing with water and Et₂O. ¹H NMR (401 MHz, DMSO-d₆) δ 12.20-11.90 (br s, 1H), 10.18 (s, 1H), 8.95 (s, 1H), 8.28 (s, 2H), 8.06-7.71 (m, 3H), 6.00 (s, 2H). LCMS R_{f}(min)=3.248, MS m/z=366.0 [M+H]⁺. HRMS (ESI) calcd for C₁₄H₁₁F₃N₇O₂⁺ [M+H]⁺ 366.0921, found 366.091.

### 62. 5-((5-(5-Cyclopropylpyridin-2-yl)oxazol-2-yl)amino)-N′-hydroxypicolinimidamide (Scheme 59)

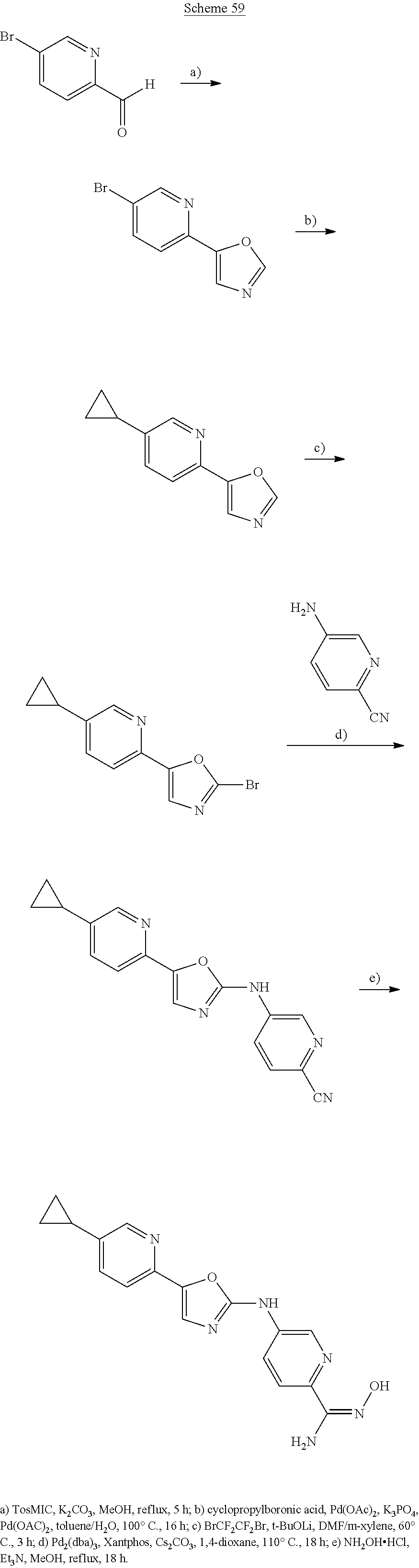

5-Bromopicolinaldehyde (3.53 g, 18.89 mmol) was reacted with TosMIC (4.26 g, 21.82 mmol) and K₂CO₃ (3.15 g, 22.78 mmol) as per General Procedure 14 in MeOH (40 mL) to provide 5-(5-bromopyridin-2-yl)oxazole (3.84 g, 90%) as a pale yellow solid.

5-(5-Bromopyridin-2-yl)oxazole (2.0 g, 8.89 mmol) was coupled with cyclopropylboronic acid (0.99, 11.56 mmol) as per General Procedure 12 Method 1 in toluene/H₂O (50/5 mL) to provide 5-(5-cyclopropylpyridin-2-yl)oxazole (1.42 g, 86%) as a white solid. ¹H NMR (401 MHz, CDCl₃) δ 8.39 (d, J=2.2 Hz, 1H), 7.90 (d, J=1.6 Hz, 1H), 7.59 (d, J=1.9 Hz, 1H), 7.49 (d, J=8.2 Hz, 1H), 7.30 (dd, J=8.2, 2.3 Hz, 1H), 1.88 (tt, J=8.4, 5.0 Hz, 1H), 1.27-0.84 (m, 2H), 0.71 (dt, J=6.6, 4.9 Hz, 2H). LCMS R_{f}(min)=3.329, MS m/z=187.1 [M+H]⁺.

5-(5-Cyclopropylpyridin-2-yl)oxazole (0.35 g, 1.88 mmol) was converted to 2-bromo-5-(5-cyclopropylpyridin-2-yl)oxazole as per General Procedure 15 Method 2 (0.26 g, 52%). ¹H NMR (401 MHz, CDCl₃) δ 8.38 (d, J=2.1 Hz, 1H), 7.53 (s, 1H), 7.45 (d, J=8.2 Hz, 1H), 7.31 (dd, J=8.2, 2.3 Hz, 1H), 1.89 (tt, J=8.5, 5.1 Hz, 1H), 1.04 (ddd, J=8.4, 6.5, 4.8 Hz, 2H), 0.73 (dt, J=6.6, 4.9 Hz, 2H). LCMS R_{f}(min)=3.354, MS m/z=265.0 [M+H]⁺. 2-Bromo-5-(5-cyclopropylpyridin-2-yl)oxazole (0.35 g, 1.32 mmol) was reacted with 5-aminonicotinonitrile (0.79 g, 6.6 mmol) following General Procedure 4 Method 1. The crude product was purified on silica gel using mixtures of DCM and EtOAc (0 to 40%) as eluents to provide the desired 5-((5-(5-cyclopropylpyridin-2-yl)oxazol-2-yl)amino)picolinonitrile in 29.2% yield (0.117 g). ¹H NMR (401 MHz, DMSO-d₆) δ 11.41 (s, 1H), 8.84 (d, J=2.6 Hz, 1H), 8.42 (d, J=1.5 Hz, 1H), 8.33 (dd, J=8.7, 2.6 Hz, 1H), 7.99 (d, J=8.7 Hz, 1H), 7.63 (s, 1H), 7.51 (s, 2H), 2.02-1.94 (m, 1H), 1.07-0.99 (m, 2H), 0.80-0.74 (m, 2H). LCMS R_{f}(min)=3.373, MS m/z=304.1 [M+H]⁺.

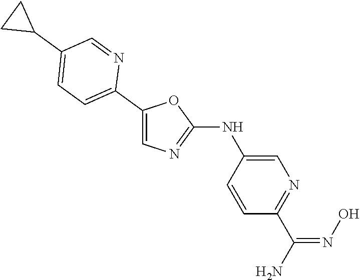 [SMILES: N/C(=N/O)c1ccc(Nc2ncc(-c3ccc(C4CC4)cn3)o2)cn1]

5-((5-(5-Cyclopropylpyridin-2-yl)oxazol-2-yl)amino)picolinonitrile (0.105 g, 0.346 mmol) was subjected to amidoxime formation in MeOH as per General Procedure 1 Method 2 to furnish 5-((5-(5-cyclopropylpyridin-2-yl)oxazol-2-yl)amino)-N′-hydroxypicolinimidamide (91.0%, 106 mg) after isolation via filtering with water, and washing with water and Et₂O. ¹H NMR (401 MHz, DMSO-d₆) δ 10.88 (s, 1H), 9.77 (s, 1H), 8.77 (s, 1H), 8.43 (s, 1H), 8.13 (d, J=7.2 Hz, 1H), 7.83 (d, J=8.7 Hz, 1H), 7.57 (s, 1H), 7.50 (s, 2H), 5.78 (s, 2H), 1.97 (m, 1H), 1.01 (m, 2H), 0.76 (m, 2H). ¹³C NMR (101 MHz, DMSO) δ 156.67, 149.86, 148.24, 144.94, 144.51, 143.39, 138.27, 137.14, 136.75, 133.51, 125.28, 124.19, 120.18, 117.99, 13.11, 9.89. LCMS R_{f}(min)=2.238, MS m/z=337.1 [M+H]⁺. HRMS (ESI) calcd for C₁₇H₁₇N₆O₂+[M+H]⁺ 337.1408, found 337.1404.

### 63. N′-Hydroxy-2-((5-(4-(trifluoromethyl)phenyl)oxazol-2-yl)amino)pyrimidine-5-carboximidamide (Scheme 60)

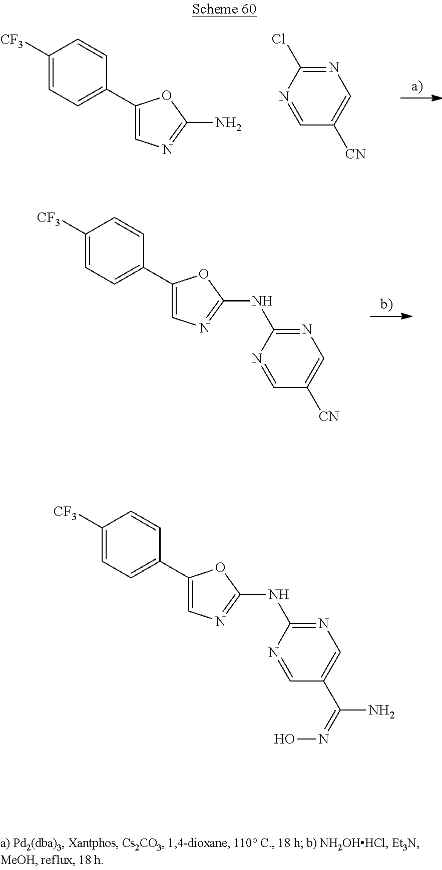 [SMILES: Nc1ncc(-c2ccc(C(F)(F)F)cc2)o1 ; N#Cc1cnc(Cl)nc1 ; N#Cc1cnc(Nc2ncc(-c3ccc(C(F)(F)F)cc3)o2)nc1 ; N/C(=N/O)c1cnc(Nc2ncc(-c3ccc(C(F)(F)F)cc3)o2)nc1]

Intermediate D (5-(4-(trifluoromethyl)phenyl)oxazol-2-amine) (0.229 g, 1 mmol) was reacted with 2-chloropyrimidine-5-carbonitrile (0.14 g, 1 mmol) following General Procedure 4 Method 1. Upon completion, the volatile solvents were removed in vacuo, and the solid residue, triturated with water, filtered and washed well with water. The resultant crude product was washed with Et₂O and DCM until all catalysts and ligands were removed, providing the 2-((5-(4-(trifluoromethyl)phenyl)oxazol-2-yl)amino)pyrimidine-5-carbonitrile in 65.6% yield (0.218 g). ¹H NMR (401 MHz, DMSO) δ 12.07-11.63 (br s, 1H), 8.93 (s, 2H), 7.82 (m, 4H), 7.79 (s, 1H). LCMS R_{f}(min)=2.842, MS m/z=332.1 [M+H]⁺.

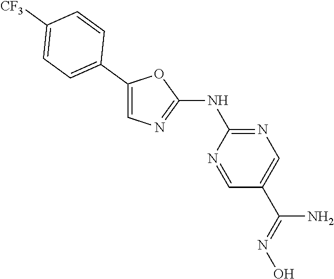 [SMILES: N/C(=N\O)c1cnc(Nc2ncc(-c3ccc(C(F)(F)F)cc3)o2)nc1]

2-((5-(4-(Trifluoromethyl)phenyl)oxazol-2-yl)amino)pyrimidine-5-carbonitrile (0.20 g, 0.603 mmol) was subjected to amidoxime formation in MeOH as per General Procedure 1 Method 2 to furnish N′-hydroxy-2-((5-(4-(trifluoromethyl)phenyl)oxazol-2-yl)amino)pyrimidine-5-carboximidamide (80.5%, 177 mg) after isolation via filtering with water, and washing with water and Et₂O. ¹H NMR (401 MHz, DMSO-d₆) δ 11.28 (s, 1H), 9.81 (s, 1H), 8.81 (s, 2H), 7.82 (s, 5H), 6.02 (s, 2H). ¹³C NMR (101 MHz, DMSO-d₆) δ 158.85, 155.84, 155.28, 147.61, 131.97, 128.04, 127.72, 126.55, 126.51, 126.00, 125.67, 123.88, 123.30, 120.98. LCMS R_{f}(min)=3.415, MS m/z=365.1 [M+H]⁺. HRMS (ESI) calcd for C₁₅H₁₂F₃N₆O₂⁺ [M+H]⁺ 365.0968, found 365.0967.

### 64. 5-((5-(4-(3,3-Difluoroazetidin-1-yl)phenyl)oxazol-2-yl)amino)-N′-hydroxypicolinimidamide (Scheme 61)

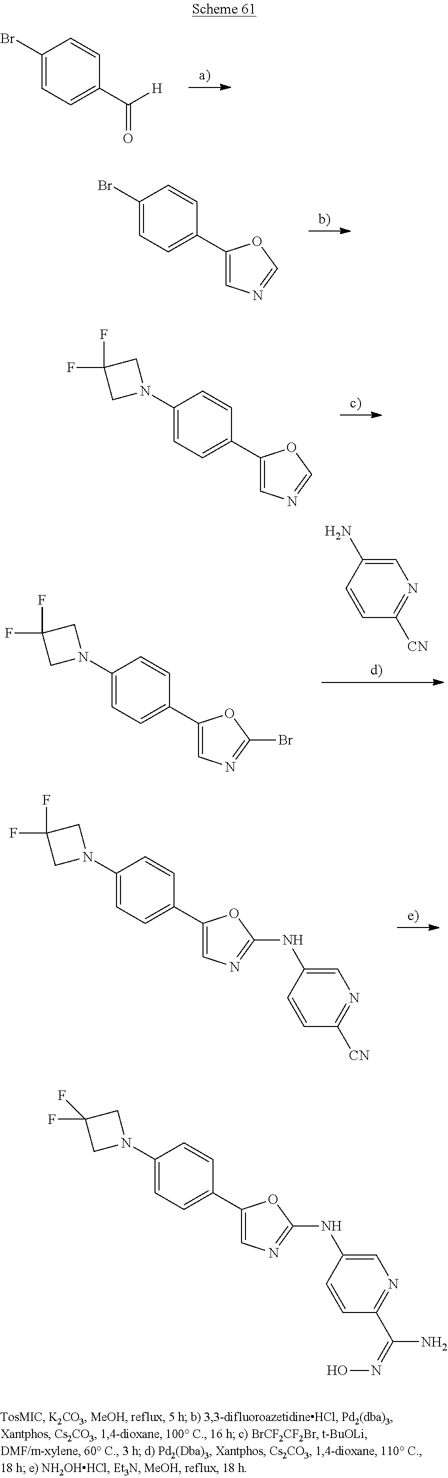

4-Bromobenzaldehyde (5.0 g, 27.02 mmol) was reacted with TosMIC (5.80 g, 29.73 mmol) and K₂CO₃ (4.48 g, 32.4 mmol) in MeOH (80 mL) as per General Procedure 14 to provide 5-(4-bromophenyl)oxazole (5.26 g, 87%) as a white solid.

5-(4-Bromophenyl)oxazole (1.0 g, 4.46 mmol) was reacted with 3,3-difluoroazetidine-HCl (0.694 g, 5.36 mmol), in the presence of Pd₂(dba)₃ (0.327 g, 0.36 mmol), Xantphos (0.232 g, 0.40 mmol) and Cs₂CO₃ (4.36 g, 13.38 mmol) in 1,4-dioxane (10 mL) at 100° C. for 16 h as per General Procedure 4 Method 1 to provide 5-(4-(3,3-difluoroazetidin-1-yl)phenyl)oxazole (0.875 g, 83%). ¹H NMR (401 MHz, CDCl₃) δ 7.85 (s, 1H), 7.55 (d, J=8.8 Hz, 2H), 7.20 (s, 1H), 6.53 (d, J=8.8 Hz, 2H), 4.27 (t, J=11.7 Hz, 4H). LCMS R_{f}(min)=2.824, MS m/z=237.1 [M+H]⁺.

5-(4-(3,3-Difluoroazetidin-1-yl)phenyl)oxazole (0.80 g, 3.37 mmol) reacted with BrCF₂CF₂Br (0.64 mL, 5.39 mmol) and t-BuOLi (0.405 g, 5.06 mmol) in DMF/m-xylene (10/10 mL) at 60° C. for 3 h as per General Procedure 15 Method 2 to provide 2-bromo-5-(4-(3,3-difluoroazetidin-1-yl)phenyl)oxazole (0.50 g, 47%) as a yellow solid. ¹H NMR (401 MHz, CDCl₃) δ 7.49 (d, J=8.8 Hz, 2H), 7.14 (s, 1H), 6.52 (d, J=8.8 Hz, 2H), 4.27 (t, J=11.7 Hz, 4H). LCMS R_{f}(min)=3.587. MS m/z=314.9 [M+H]⁺.

2-Bromo-5-(4-(3,3-difluoroazetidin-1-yl)phenyl)oxazole) (0.28 g, 0.888 mmol) was reacted with 5-aminonicotinonitrile (0.53 g, 4.44 mmol) following General Procedure 4 Method 1. The crude product was purified on silica gel using mixtures of DCM and EtOAc (0 to 30%) and then preparative HPLC to provide the 5-((5-(4-(3,3-difluoroazetidin-1-yl)phenyl)oxazol-2-yl)amino)picolinonitrile in 18.15% yield (0.057 g). ¹H NMR (401 MHz, DMSO) δ 11.16 (s, 1H), 8.80 (s, 1H), 8.30 (d, J=6.3 Hz, 1H), 7.94 (d, J=7.4 Hz, 1H), 7.48 (d, J=6.3 Hz, 2H), 7.33 (s, 1H), 6.63 (d, J=6.3 Hz, 2H), 4.30 (t, J=11.2 Hz, 4H). LCMS R_{f}(min)=2.986, MS m/z=354.1 [M+H]⁺.

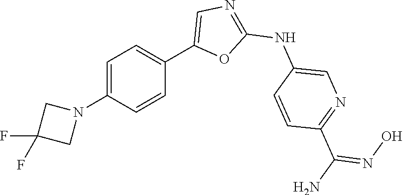 [SMILES: N/C(=N/O)c1ccc(Nc2ncc(-c3ccc(N4CC(F)(F)C4)cc3)o2)cn1]

5-((5-(4-(3,3-Difluoroazetidin-1-yl)phenyl)oxazol-2-yl)amino)picolinonitrile (0.055 g, 0.155 mmol) was subjected to amidoxime formation in MeOH as per General Procedure 1 Method 2 to furnish 5-((5-(4-(3,3-difluoroazetidin-1-yl)phenyl)oxazol-2-yl)amino)-N′-hydroxypicolinimidamide (76.5%, 46 mg) after isolation via filtering with water, and washing with water and Et₂O. ¹H NMR (401 MHz, DMSO-d₆) δ 10.63 (s, 1H), 9.71 (s, 1H), 8.76 (s, 1H), 8.11 (d, J=8.5 Hz, 1H), 7.82 (d, J=8.4 Hz, 1H), 7.48 (d, J=7.5 Hz, 2H), 7.29 (s, 1H), 6.65 (d, J=7.7 Hz, 2H), 5.75 (s, 2H), 4.30 (t, J=12.0 Hz, 4H). ¹³C NMR (101 MHz, DMSO-d₆) δ 155.45, 149.88, 149.49, 145.29, 143.06, 137.02, 136.93, 124.36, 123.88, 120.33, 120.13, 119.85, 119.04, 117.13, 114.42, 113.60, 63.59, 63.34, 63.09. LCMS R_{f}(min)=2.45, MS m/z=387.1 [M+H]⁺. HRMS (ESI) calcd for C₁₈H₁₇F₂N₆O₂⁺ [M+H]⁺ 387.1376, found 387.1373.

### 65. N′-Hydroxy-5-((5-(5-(trifluoromethyl)pyrimidin-2-yl)oxazol-2-yl)amino)picolinimidamide (Scheme 62)

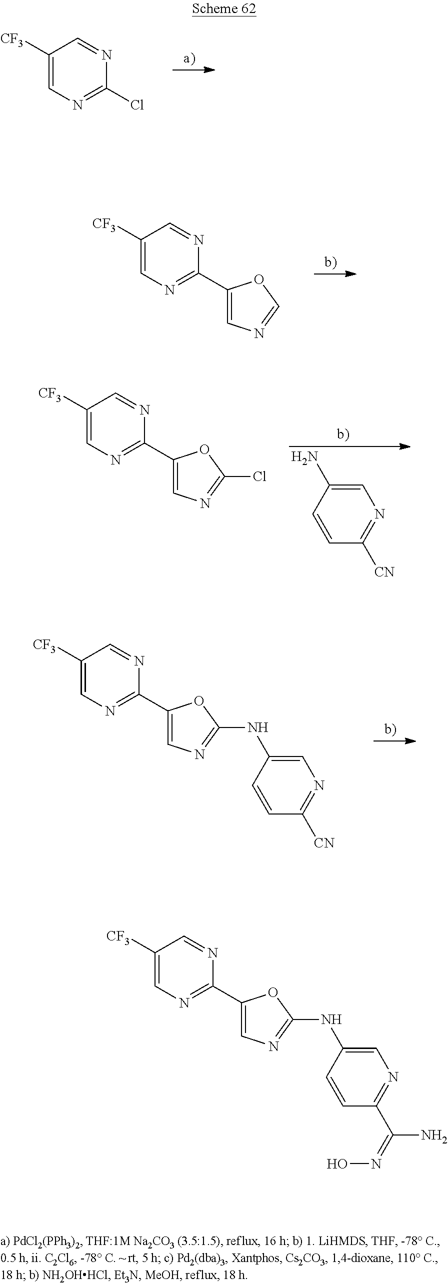 [SMILES: FC(F)(F)c1cnc(Cl)nc1 ; FC(F)(F)c1cnc(-c2cnco2)nc1 ; FC(F)(F)c1cnc(-c2cnc(Cl)o2)nc1 ; N#Cc1ccc(N)cn1 ; N#Cc1ccc(Nc2ncc(-c3ncc(C(F)(F)F)cn3)o2)cn1 ; N/C(=N/O)c1ccc(Nc2ncc(-c3ncc(C(F)(F)F)cn3)o2)cn1]

To a degassed biphasic solution of THF (3.5 mL) and 1 M Na₂CO₃ (1.5 mL), was added 2-chloro-5-(trifluoromethyl)pyrimidine (876 mg, 4.80 mmol, 1.0 eq.), 5-(4,4,5,5-tetramethyl-1,3,2-dioxaborolan-2-yl)oxazole (1.0 M in THF, 7.20 mL, 7.20 mmol, 1.5 eq.) and PdCl₂(PPh₃)₂ (835 mg, 0.720 mmol, 0.15 eq.). The mixture was reacted according to General Procedure 12 Method 1 to afford 5-(5-(trifluoromethyl)pyrimidin-2-yl)oxazole as a brown solid (53 mg, 5.1%). ¹H NMR (401 MHz, CDCl₃) δ 9.02 (s, 2H), 8.12 (s, 1H), 8.07 (s, 1H); ¹³C NMR (101 MHz, CDCl₃) δ 155.1, 153.5, 149.0, 132.0, 132.0, 126.8, 124.1, 123.3, 122.9, 121.4, 118.6 ppm. LCMS R_{f}(min)=3.344, MS m/z=216.1 [M+H]⁺.

5-(5-(Trifluoromethyl)pyrimidin-2-yl)oxazole (0.205 g, 0.95 mmol) was reacted with LiHMDS (1.05 mL, 1.05 mmol, 1 M in hexane) and C₂Cl₆ (0.339 g, 1.43 mmol) in THF (10 mL) as per General Procedure 15 Method 1 to provide 2-chloro-5-(5-(trifluoromethyl)pyrimidin-2-yl)oxazole (0.206 g, 87%) as a white solid. ¹H NMR (401 MHz, CDCl₃) δ 9.01 (d, J=0.6 Hz, 2H), 7.98 (s, 1H). LCMS R_{f}(min)=2.871, MS m/z=250.0 [M+H]⁺.

2-Chloro-5-(5-(trifluoromethyl)pyrimidin-2-yl)oxazole (0.3 g, 1.2 mmol) was reacted with 5-aminonicotinonitrile (0.43 g, 3.6 mmol) following General Procedure 4 Method 1. The crude product was purified on preparative HPLC to provide the 5-((5-(5-(trifluoromethyl)pyrimidin-2-yl)oxazol-2-yl)amino)picolinonitrile in 9.3% yield (0.037 g). ¹H NMR (401 MHz, DMSO-d₆) δ 11.81 (s, 1H), 9.22 (s, 2H), 8.85 (d, J=2.3 Hz, 1H), 8.31 (dd, J=8.6, 2.5 Hz, 1H), 8.08 (s, 1H), 8.02-8.00 (m, 1H). LCMS R_{f}(min)=3.805, MS m/z=333.1 [M+H]⁺.

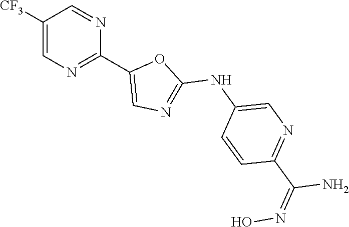 [SMILES: N/C(=N/O)c1ccc(Nc2ncc(-c3ncc(C(F)(F)F)cn3)o2)cn1]

5-((5-(5-(Trifluoromethyl)pyrimidin-2-yl)oxazol-2-yl)amino)picolinonitrile (0.037 g, 0.11 mmol) was subjected to amidoxime formation in MeOH as per General Procedure 1 Method 2 to furnish N′-hydroxy-5-((5-(5-(trifluoromethyl)pyrimidin-2-yl)oxazol-2-yl)amino)picolinimidamide (71.3%, 29 mg) after isolation via filtering with water, and washing with water and Et₂O. ¹H NMR (401 MHz, DMSO-d₆) δ 11.69 (s, 1H), 10.91 (br s, 1H), 9.22 (d, J=0.8 Hz, 2H), 8.88 (d, J=2.5 Hz, 1H), 8.29 (dd, J=8.8, 2.6 Hz, 1H), 8.08 (s, 1H), 8.03 (d, J=8.8 Hz, 1H). ¹³C NMR (101 MHz, DMSO-d₆) δ 158.50, 155.81, 154.53, 143.26, 138.88, 138.54, 134.27, 124.96, 124.23, 123.15, 122.26, 120.84, 120.51. LCMS R_{f}(min)=3.037, MS m/z=366.1 [M+H]⁺. HRMS (ESI) calcd for C₁₄H₁₁F₃N₇O₂⁺ [M+H]⁺ 366.0921, found 366.0918.

### 66. N′-Hydroxy-5-((5-(5-(trifluoromethyl)pyrazin-2-yl)oxazol-2-yl)amino)picolinimidamide (Scheme 63)

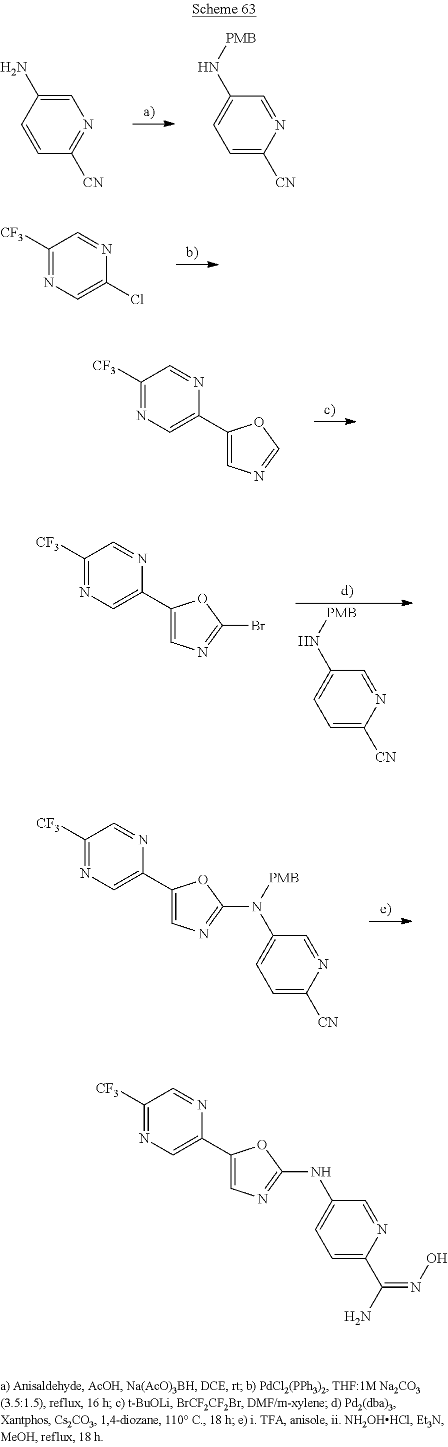

5-Aminonicotinonitrile (5 g, 41.97 mmol) and anisaldehyde (5.45 g, 46.16 mmol) was dissolved in DCE (100 mL), AcOH (7.2 mL, 126 mmol) added, and the reaction mixture was stirred at rt overnight. NaBH(AcO)₃ (17.8 g, 83.9 mmol) was added in portions every hr, until most of the starting amine has been consumed as evidenced by TLC. The reaction mixture was diluted with DCM (100 mL), poured slowly into saturated NaHCO₃ solution (200 mL). The aqueous phase was made alkaline (pH˜8) by addition of solid NaHCO₃. After separation, the aqueous phase was extracted with more DCM (3×). The combined organic phase was dried over MgSO₄, evaporated to dryness. The residue was purified on silica gel using mixtures of DCM and EtOAc (0 to 10%) as eluents to provide 5-((4-methoxybenzyl)amino)picolinonitrile in 51.8% yield (5.2 g). ¹H NMR (401 MHz, CDCl₃) δ 8.06 (d, J=2.8 Hz, 1H), 7.43 (d, J=8.6 Hz, 1H), 7.26-7.21 (m, 2H), 6.92-6.86 (m, 2H), 6.82 (dd, J=8.6, 2.9 Hz, 1H), 4.32 (s, 2H), 3.80 (s, 3H). LCMS R_{f}(min)=2.772, MS m/z=240.1 [M+H]⁺.

To a degassed biphasic solution of THF (3.5 mL) and 1 M Na₂CO₃ (1.5 mL), was added 2-chloro-5-(trifluoromethyl)pyrazine (150 mg, 0.822 mmol, 1.0 eq.), 5-(4,4,5,5-tetramethyl-1,3,2-dioxaborolan-2-yl)oxazole (1.0 M in THF, 904 μL, 0.904 mmol, 1.1 eq.) and PdCl₂(PPh₃)₂ (58 mg, 0.082 mmol, 0.1 eq.). The mixture was reacted according to General Procedure 12 Method 1 to afford 5-(5-(trifluoromethyl)pyrazin-2-yl)oxazole as a brown solid (53 mg, 30%). ¹H NMR (401 MHz, CDCl₃) δ 9.02 (s, 1H), 8.92 (s, 1H), 8.10 (s, 1H), 7.92 (s, 1H); ¹³C NMR (101 MHz, CDCl₃) δ 152.8, 148.0, 145.5, 143.0, 142.6, 142.3, 141.9, 141.5, 141.5, 141.5, 141.4, 140.0, 129.1, 125.2, 122.5, 119.7, 117.0 ppm. LCMS R_{f}(min)=3.223, MS m/z=216.0 [M+H]⁺.

5-(5-(Trifluoromethyl)pyrazin-2-yl)oxazole (0.25 g, 1.16 mmol) was reacted with BrCF₂CF₂Br (0.21 mL, 1.74 mmol) and t-BuOLi (0.121 g, 1.51 mmol) in DMF/m-xylene (3/3 mL) as per General Procedure 15 Method 2 to provide 2-bromo-5-(5-(trifluoromethyl)pyrazin-2-yl)oxazole (0.232 g, 67%) as a yellow solid. ¹H NMR (401 MHz, CDCl₃) δ 8.98 (d, J=1.1 Hz, 1H), 8.93 (d, J=1.0 Hz, 1H), 7.86 (s, 1H). LCMS R_{f}(min)=2.939, MS m/z=294.0 [M+H]⁺.

2-Bromo-5-(5-(trifluoromethyl)pyrazin-2-yl)oxazole) (0.22 g, 0.748 mmol) was reacted with 5-((4-methoxybenzyl)amino)picolinonitrile (0.42 g, 1.76 mmol) following General Procedure 4 Method 1. The crude product was purified on silica gel using mixtures of DCM and EtOAc (0 to 15%) as eluents to provide the 5-((4-methoxybenzyl)(5-(5-(trifluoromethyl)pyrazin-2-yl)oxazol-2-yl)amino)picolinonitrile in 54.9% yield (0.186 g). ¹H NMR (401 MHz, CDCl₃) δ 8.86 (d, J=2.3 Hz, 1H), 8.83 (d, J=1.0 Hz, 1H), 8.73 (d, J=1.1 Hz, 1H), 8.01 (dd, J=8.6, 2.8 Hz, 1H), 7.77 (s, 1H), 7.69 (dd, J=8.6, 0.5 Hz, 1H), 7.20 (d, J=8.7 Hz, 2H), 6.88-6.85 (m, 2H), 5.29 (s, 2H), 3.78 (s, 3H). LCMS R_{f}(min)=3.579, MS m/z=453.1 [M+H]⁺.

5-((4-Methoxybenzyl)(5-(5-(trifluoromethyl)pyrazin-2-yl)oxazol-2-yl)amino)picolinonitrile (0.186 g, 0.408 mmol) was deprotected with TFA and anisole mixture as per General Procedure 11. The PMB-deprotected product, which was collected via trituration with Et₂O (LCMS R_{f} (min)

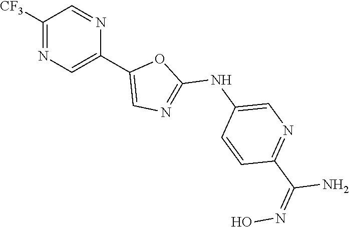 [SMILES: N/C(=N/O)c1ccc(Nc2ncc(-c3cnc(C(F)(F)F)cn3)o2)cn1]

=2.843, MS m/z=333.1 [M+H]⁺ was subjected to amidoxime formation in MeOH as per General Procedure 1 Method 2 to furnish N′-hydroxy-5-((5-(5-(trifluoromethyl)pyrazin-2-yl)oxazol-2-yl)amino)picolinimidamide (63.6%, 95 mg) after purification with preparative HPLC. ¹H NMR (401 MHz, DMSO-d₆) δ 11.26 (s, 1H), 9.77 (s, 1H), 9.08 (s, 2H), 8.78 (d, J=2.1 Hz, 1H), 8.16-8.08 (m, 2H), 7.86 (d, J=8.8 Hz, 1H), 5.77 (s, 2H). ¹³C NMR (101 MHz, DMSO-d₆) δ 158.77, 149.77, 146.10, 144.13, 141.77, 141.74, 140.11, 139.38, 139.03, 138.68, 138.34, 137.60, 136.14, 132.35, 124.83, 123.35, 120.63, 120.24. LCMS R_{f}(min)=2.326, MS m/z=366.0 [M+H]⁺. HRMS (ESI) calcd for C₁₄H₁₁F₃N₇O₂⁺ [M+H]⁺ 366.0921, found 366.0924.

### 67. 6-((5-(4-(Trifluoromethoxy)phenyl)-1,3,4-oxadiazol-2-yl)amino)pyridin-3-ol (Scheme 64)

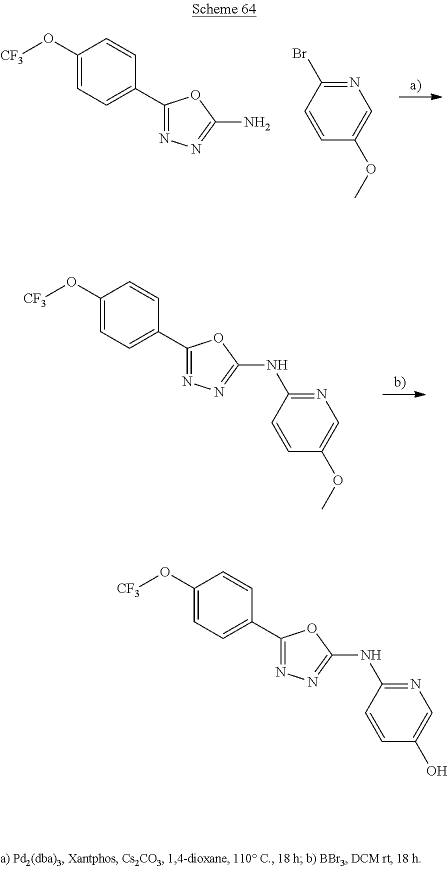

5-(4-(Trifluoromethoxy)phenyl)-1,3,4-oxadiazol-2-amine (0.58 g, 0.2.36 mmol) was reacted with 2-bromo-5-methoxypyridine (0.89 g, 4.73 mmol) following General Procedure 4 Method 1. The crude product was triturated, filtered, and washed well with water. Excess of catalyst and ligands were removed by washing with Et₂O and DCM, providing the N-(5-methoxypyridin-2-yl)-5-(4-(trifluoromethoxy)phenyl)-1,3,4-oxadiazol-2-amine in 31.3% yield (0.261 g). ¹H NMR (401 MHz, DMSO-d₆) δ 11.11 (s, 1H), 8.05 (s, 1H), 8.00 (d, J=8.3 Hz, 2H), 7.88 (d, J=8.9 Hz, 1H), 7.59 (d, J=7.9 Hz, 2H), 7.52 (d, J=6.9 Hz, 1H), 3.81 (s, 3H). LCMS R_{f}(min)=3.227, MS m/z=353.1 [M+H]⁺.

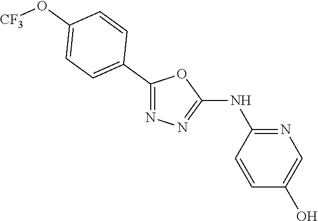 [SMILES: Oc1ccc(Nc2nnc(-c3ccc(OC(F)(F)F)cc3)o2)nc1]

N-(5-Methoxypyridin-2-yl)-5-(4-(trifluoromethoxy)phenyl)-1,3,4-oxadiazol-2-amine (0.26 g, 0.738 mmol) was deprotected with BBr₃ in DCM as per General Procedure 8. The crude product was purified on preparative HPLC to furnish 6-((5-(4-OH (trifluoromethoxy)phenyl)-1,3,4-oxadiazol-2-yl)amino)pyridin-3-ol (73.3%, 183 mg). ¹H NMR (401 MHz, DMSO-d₆) δ 10.94 (s, 1H), 9.60 (s, 1H), 7.99 (d, J=8.6 Hz, 2H), 7.88 (d, J=2.2 Hz, 1H), 7.77 (d, J=8.8 Hz, 1H), 7.58 (d, J=8.2 Hz, 2H), 7.29 (dd, J=8.8, 2.6 Hz, 1H). ¹³C NMR (101 MHz, DMSO-d₆) δ 160.48, 157.54, 150.28, 149.90, 144.34, 135.51, 128.22, 125.66, 123.53, 122.29, 112.64. LCMS R_{f}(min)=2.691, MS m/z=339.1 [M+H]⁺. HRMS (ESI) calcd for C₁₄H₁₀F₃N₄O₃⁺ [M+H]⁺ 339.07, found 339.0704.

### 68. 1-Hydroxy-4-((5-(5-(trifluoromethyl)pyridin-2-yl)oxazol-2-yl)amino)pyridin-2(1H)-one (Scheme 65)

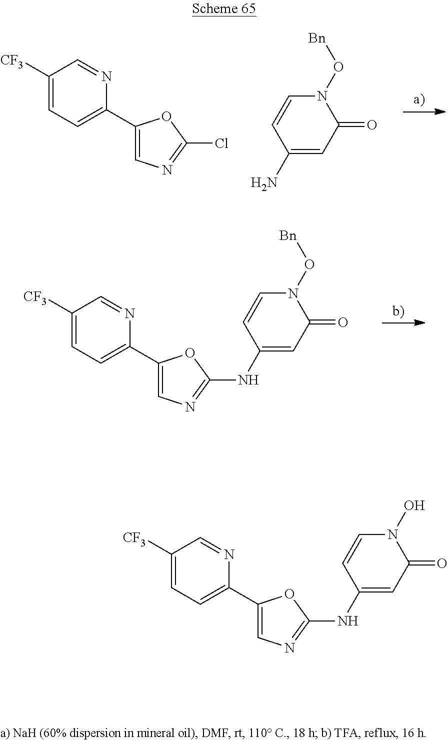 [SMILES: FC(F)(F)c1ccc(-c2cnc(Cl)o2)nc1 ; Nc1ccn(OCc2ccccc2)c(=O)c1 ; O=c1cc(Nc2ncc(-c3ccc(C(F)(F)F)cn3)o2)ccn1OCc1ccccc1 ; O=c1cc(Nc2ncc(-c3ccc(C(F)(F)F)cn3)o2)ccn1O]

2-Chloro-5-(5-(trifluoromethyl)pyridin-2-yl)oxazole (Intermediate J, 0.741 g, 2.98 mmol) was reacted with 4-amino-1-(benzyloxy)pyridin-2(1H)-one (0.645 g, 2.98 mmol) following General Procedure 2 Method 2. The crude product was triturated with water and DCM to provide the 1-(benzyloxy)-4-((5-(5-(trifluoromethyl)pyridin-2-yl)oxazol-2-yl)amino)pyridin-2(1H)-one in 50% yield (0.624 g). ¹H NMR (401 MHz, DMSO-d₆) δ 11.09 (s, 1H), 8.96-8.92 (m, 1H), 8.28 (dd, J=8.5, 1.8 Hz, 1H), 7.92 (s, 1H), 7.81 (d, J=8.4 Hz, 1H), 7.68 (d, J=7.8 Hz, 1H), 7.48 (m, 2H), 7.41 (m, 3H), 6.99 (d, J=2.8 Hz, 1H), 6.22 (dd, J=7.8, 2.9 Hz, 1H), 5.18 (s, 2H). LCMS R_{f}(min)=3.653, MS m/z=429.1 [M+H]⁺.

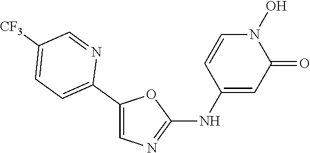 [SMILES: O=c1cc(Nc2ncc(-c3ccc(C(F)(F)F)cn3)o2)ccn1O]

1-(Benzyloxy)-4-((5-(5-(trifluoromethyl)pyridin-2-yl)oxazol-2-yl)amino)pyridin-2(1H)-one (0.2 g, 0.47 mmol) was stirred in TFA (10 mL) at reflux for 16 h. The volatile solvents were removed in vacuo, and the residual crude product was purified on preparative HPLC to furnish 1-hydroxy-4-((5-(5-(trifluoromethyl)pyridin-2-yl)oxazol-2-yl)amino)pyridin-2(1H)-one (48%, 76 mg). ¹H NMR (401 MHz, DMSO-d₆) δ 11.05 (s, 1H), 8.94 (d, J=0.9 Hz, 1H), 8.27 (dd, J=8.5, 1.9 Hz, 1H), 7.92 (s, 1H), 7.82 (d, J=2.5 Hz, 1H), 7.80 (d, J=3.3 Hz, 1H), 6.95 (d, J=2.8 Hz, 1H), 6.33 (dd, J=7.7, 2.8 Hz, 1H). LCMS R_{f}(min)=3.204, MS m/z=339.1 [M+H]⁺. HRMS (ESI) calcd for C₁₄H₁₀F₃N₄O₃⁺ [M+H]⁺ 339.07, found 339.0706.

### 69. N′-Hydroxy-5-((5-(5-(trifluoromethyl)pyridin-2-yl)-1,3,4-oxadiazol-2-yl)amino)picolinimidamide (Scheme 66)

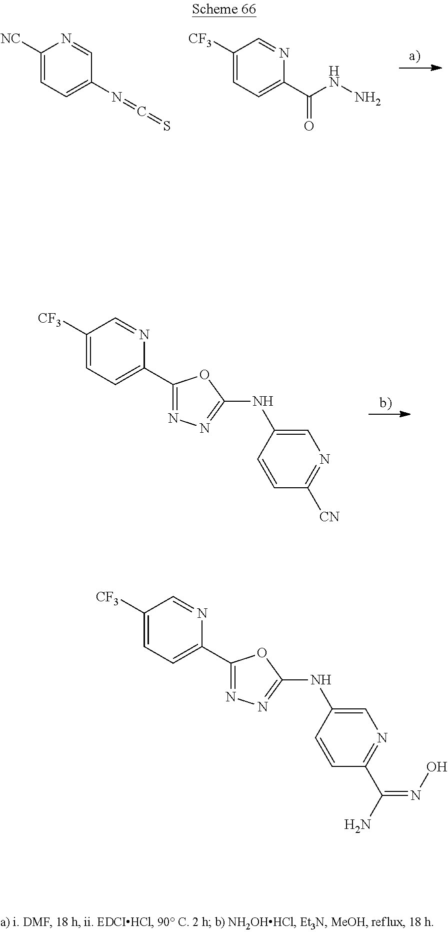 [SMILES: N#Cc1ccc(N=C=S)cn1 ; NNC(=O)c1ccc(C(F)(F)F)cn1 ; N#Cc1ccc(Nc2nnc(-c3ccc(C(F)(F)F)cn3)o2)cn1 ; N/C(=N/O)c1ccc(Nc2nnc(-c3ccc(C(F)(F)F)cn3)o2)cn1]

5-Isothiocyanatopicolinonitrile (0.161 g, 1.01 mmol) was reacted with 5-(trifluoromethyl)picolinohydrazide (0.173 g, 0.843 mmol) following General Procedure 7. The crude product was purified on silica gel using mixtures of DCM and EtOAc (0 to 30%) as eluents to provide 5-((5-(5-(trifluoromethyl)pyridin-2-yl)-1,3,4-oxadiazol-2-yl)amino)picolinonitrile in 81% yield (0.227 g). ¹H NMR (401 MHz, DMSO-d₆) δ 11.93 (br s, 1H), 9.18-9.15 (m, 1H), 8.85-8.83 (m, 1H), 8.45 (dd, J=8.4, 1.8 Hz, 1H), 8.34-8.26 (m, 2H), 8.08 (dd, J=8.6, 0.5 Hz, 1H). LCMS R_{f}(min)=3.450, MS m/z=333.1 [M+H]⁺.

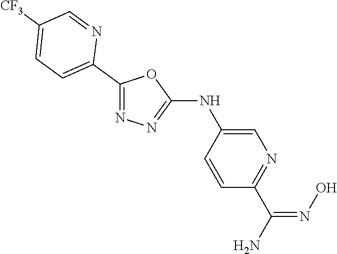 [SMILES: N/C(=N/O)c1ccc(Nc2nnc(-c3ccc(C(F)(F)F)cn3)o2)cn1]

5-((5-(5-(Trifluoromethyl)pyridin-2-yl)-1,3,4-oxadiazol-2-yl)amino)picolinonitrile (0.212 g, 0.638 mmol) was subjected to amidoxime formation in MeOH as per General Procedure 1 Method 2 to furnish N′-hydroxy-5-((5-(5-(trifluoromethyl)pyridin-2-yl)-1,3,4-oxadiazol-2-yl)amino)picolinimidamide hydrochloride (38.2%, 98 mg) after filtering and washing with water, then washing with MeOH, Et₂O, and finally freeze-drying from dioxane with 1.5 equivalents of HCl as 4 M dioxane solution. ¹H NMR (401 MHz, DMSO-d₆) δ 11.95 (s, 1H), 11.04 (br s, 1H), 9.17 (s, 1H), 8.95 (d, J=2.5 Hz, 1H), 8.97-8.69 (br s, 2H), 8.46 (dd, J=8.5, 1.9 Hz, 1H), 8.35-8.24 (m, 3H), 8.17 (d, J=8.8 Hz, 1H). LCMS R_{f}(min)=3.019, MS m/z=366.1 [M+H]⁺. HRMS (ESI) calcd for C₁₄H₁₁F₃N₇O₂⁺ [M+H]⁺ 366.0921, found 366.092.

### 70. N′-Hydroxy-5-((5-(4-(trifluoromethoxy)phenyl)oxazol-2-yl)amino)pyrazine-2-carboximidamide (Scheme 67)

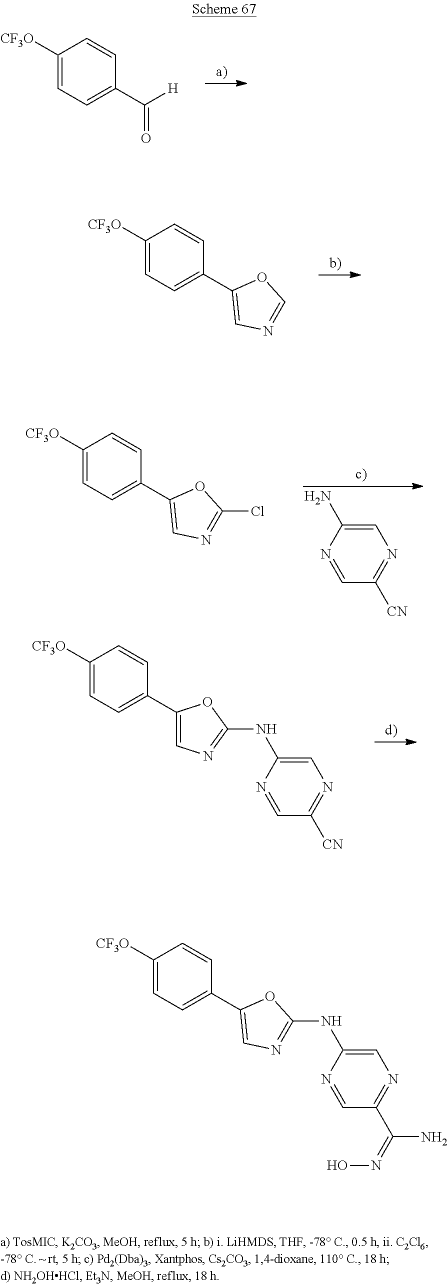

4-(Trifluoromethoxy)benzaldehyde (5.0 g, 26.3 mmol) was reacted TosMIC (5.65 g, 18.93 mmol) and K₂CO₃ (4.36 g, 31.56 mmol) in MeOH (60 mL) as per General Procedure 14 to provide 5-(4-(trifluoromethoxy)phenyl)oxazole (5.18 g, 86%) as a white solid. ¹H NMR (401 MHz, CDCl₃) δ 7.93 (s, 1H), 7.73-7.62 (m, 2H), 7.36 (s, 1H), 7.32-7.24 (m, 2H). LCMS R_{f}(min)=3.046, MS m/z=230.0 [M+H]⁺.

5-(4-(Trifluoromethoxy)phenyl)oxazole (2.0 g, 8.72 mmol) was reacted with LiHMDS (9.6 mL, 9.6 mmol, 1 M in hexane) and C₂Cl₆ (3.11 g, 13.13 mmol) in THF (20 mL) as per General Procedure 15 Method 1 to provide 2-chloro-5-(4-(trifluoromethoxy)phenyl)oxazole (1.85 g, 80%) as a white solid. ¹H NMR (401 MHz, CDCl₃) δ 7.61-7.58 (m, 2H), 7.59-7.56 (m, 2H), 7.24 (s, 1H). LCMS R_{f}(min)=3.328, MS m/z=264.0 [M+H]⁺.

2-Chloro-5-(4-(trifluoromethoxy)phenyl)oxazole) (1.2 g, 4.55 mmol) was reacted with 5-aminopyrazine-2-carbonitrile (0.656 g, 5.46 mmol) following General Procedure 4 Method 1. The crude product was purified on silica gel with mixtures of EtOAc (0 to 60%) in DCM as eluents, then on preparative HPLC to provide the 5-((5-(4-(trifluoromethoxy)phenyl)oxazol-2-yl)amino)pyrazine-2-carbonitrile in 12.5% yield (0.197 g). ¹H NMR (401 MHz, DMSO-d₆) δ 8.41 (br s, 1H), 7.84 (s, 1H), 6.95 (d, J=8.7 Hz, 2H), 6.67 (s, 1H), 6.54 (d, J=8.3 Hz, 2H). LCMS R_{f} (min)=3.859, MS m/z=348.1 [M+H]⁺.

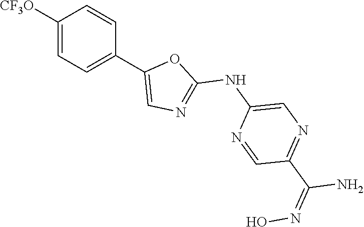

5-((5-(4-(Trifluoromethoxy)phenyl)oxazol-2-yl)amino)pyrazine-2-carbonitrile (0.1 g, 0.287 mmol) was subjected to amidoxime formation in MeOH as per General Procedure 1 Method 2 to furnish N′-hydroxy-5-((5-(4-(trifluoromethoxy)phenyl)oxazol-2-yl)amino)pyrazine-2-carboximidamide (44.7%, 49 mg) after isolation via filtering with water, and washing with water, MeOH, and Et₂O. ¹H NMR (401 MHz, DMSO-d₆) δ 11.52 (s, 1H), 9.95 (s, 1H), 9.24 (s, 1H), 8.74 (d, J=1.1 Hz, 1H), 7.74 (d, J=8.7 Hz, 2H), 7.65 (s, 1H), 7.47 (d, J=8.2 Hz, 2H), 5.88 (s, 2H). LCMS R_{f}(min)=3.425, MS m/z=381.1 [M+H]⁺. HRMS (ESI) calcd for C₁₅H₁₂F₃N₆O₃⁺ [M+H]⁺ 381.0917, found 381.0925.

### 71. 5-((5-(5-Chloropyridin-2-yl)-1,3,4-oxadiazol-2-yl)amino)-N′-hydroxypicolinimidamide (Scheme 68)

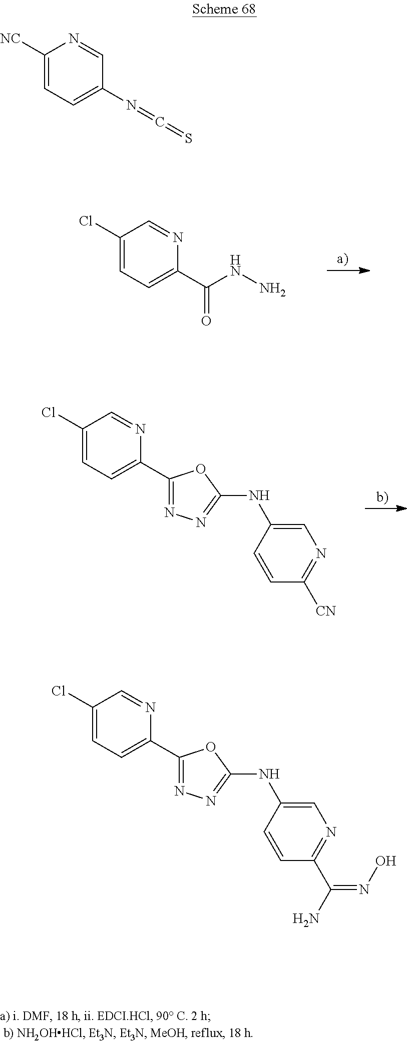 [SMILES: N#Cc1ccc(N=C=S)cn1 ; NNC(=O)c1ccc(Cl)cn1 ; N#Cc1ccc(Nc2nnc(-c3ccc(Cl)cn3)o2)cn1 ; N/C(=N/O)c1ccc(Nc2nnc(-c3ccc(Cl)cn3)o2)cn1]

5-Isothiocyanatopicolinonitrile (0.163 g, 1.01 mmol) was reacted with 5-chloropicolinohydrazide (0.173 g, 1.01 mmol) following General Procedure 7. The crude product was filtered with water, and washing with water, MeOH, and to provide the desired 5-((5-(5-chloropyridin-2-yl)-1,3,4-oxadiazol-2-yl)amino)picolinonitrile in 90.9% yield (0.274 g). ¹H NMR (401 MHz, DMSO) δ 11.84 (br s, 1H), 8.83 (m, 2H), 8.28 (dd, J=8.6, 2.6 Hz, 1H), 8.18 (dd, J=8.5, 2.3 Hz, 1H), 8.13 (d, J=8.5 Hz, 1H), 8.07 (d, J=8.6 Hz, 1H). LCMS R_{f}(min)=3.428, MS m/z=299.0 [M+H]⁺.

5-((5-(5-Chloropyridin-2-yl)-1,3,4-oxadiazol-2-yl)amino)picolinonitrile (0.25 g, 0.836 mmol) was subjected to amidoxime formation in MeOH as per General Procedure 1 Method 2 to furnish 5-((5-(5-chloropyridin-2-yl)-1,3,4-oxadiazol-2-yl)amino)-N′-hydroxypicolinimidamide

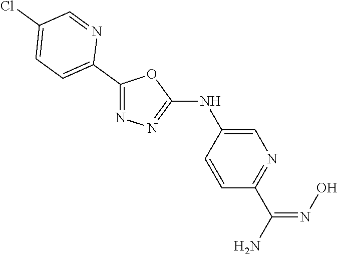 [SMILES: N/C(=N/O)c1ccc(Nc2nnc(-c3ccc(Cl)cn3)o2)cn1]

hydrochloride (83.4%, 257 mg) after filtering and washing with water, then washing with MeOH, Et₂O, and finally freeze-drying from dioxane with 1.5 equivalents of HCl as 4 M dioxane solution. ¹H NMR (401 MHz, DMSO-d₆) δ 11.65 (s, 1H), 10.52 (br s, 1H), 8.84 (m, 2H), 8.31-7.90 (m, 4H), 7.52 (br s, 2H). ¹³C NMR (101 MHz, DMSO-d₆) δ 160.55, 158.01, 149.30, 141.82, 138.41, 138.07, 133.31, 124.73, 123.58, 122.20. LCMS R_{f}(min)=2.943, MS m/z=332.0 [M+H]⁺. HRMS (ESI) calcd for C₁₃H₁₁ClN₇O₂⁺ [M+H]⁺ 332.0657, found 332.0669.

### 72. N′-Hydroxy-5-((1-(5-(trifluoromethyl)pyridin-2-yl)-1H-pyrazol-4-yl)amino)picolinimidamide (Scheme 69)

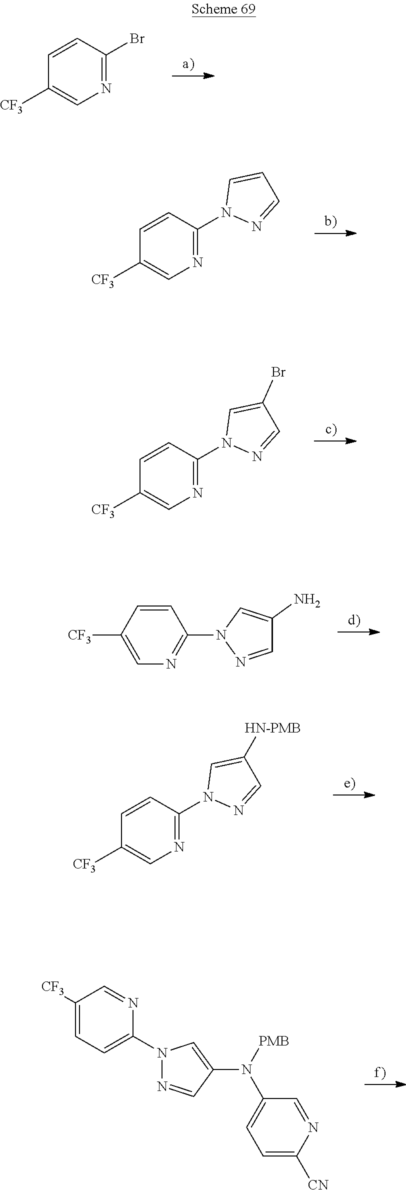

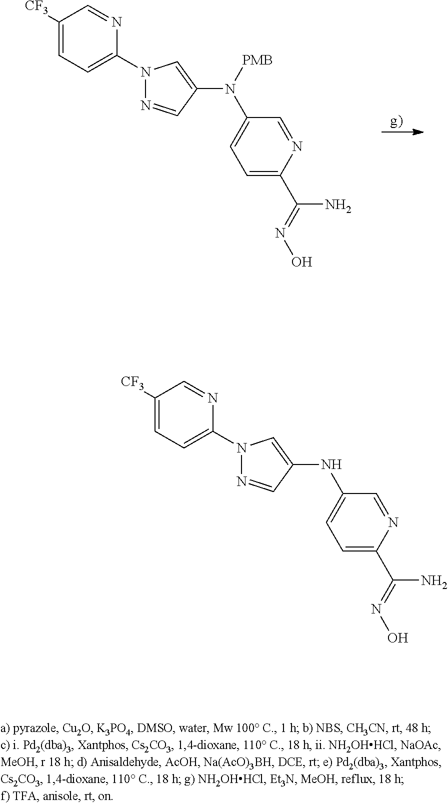

An oven-dried tube was charged with 2-bromo-5-(trifluoromethyl)pyridine (2.5 g, 11.06 mmol), pyrazole (0.98 g, 14.38 mmol), Cu₂O (0.158 g, 1.106 mmol) and K₃PO₄ (4.69 g, 22.12 mmol). DMSO (11 mL) and water (0.5 mL) were added, and the tube was sealed under nitrogen, then irradiated with microwave at 100° C. for 1 h. The reaction mixture was cooled to room temperature, diluted with Et₂O and half-brine. After separation the aqueous phase was extracted with Et₂O (4×), the combined organic phases were dried over (MgSO₄), evaporated to dryness. The residue was purified on silica gel using mixtures of petroleum spirits and DCM as eluents to provide 2-(1H-pyrazol-1-yl)-5-(trifluoromethyl)pyridine in 53.9% yield (1.8 g). ¹H NMR (401 MHz, CDCl₃) δ 8.68-8.65 (m, 1H), 8.59 (dd, J=2.7, 0.7 Hz, 1H), 8.10 (d, J=8.7 Hz, 1H), 8.03 (ddd, J=8.7, 2.3, 0.5 Hz, 1H), 7.77 (d, J=1.0 Hz, 1H), 6.50 (dd, J=2.7, 1.7 Hz, 1H). LCMS R_{f}(min)=3.879, MS m/z=214.1 [M+H]⁺.

### Intermediate M: 2-(4-bromo-1H-pyrazol-1-yl)-5-(trifluoromethyl)pyridine

2-(1H-Pyrazol-1-yl)-5-(trifluoromethyl)pyridine (1.7 g, 7.98 mmol) was stirred with NBS (3.12 g, 17.54 mmol) in CH₃CN (16 mL) at rt for 48 h. The volatile solvents were removed and the residue was taken up in DCM and saturated aq. NaHCO₃ solutions. After separation the aqueous phase was extracted with DCM (4×), the combined organic phases were dried over (MgSO₄), evaporated to dryness. The residue was purified on silica gel using mixtures of petroleum spirits and DCM as eluents to provide 2-(4-bromo-1H-pyrazol-1-yl)-5-(trifluoromethyl)pyridine in 85.9% yield (2 g). ¹H NMR (401 MHz, CDCl₃) δ 8.67-8.64 (m, 1H), 8.61 (d, J=0.7 Hz, 1H), 8.07-8.02 (m, 2H), 7.70 (s, 1H). LCMS R_{f}(min)=4.319, MS m/z=292.0/294.0 [M+H]⁺.

Intermediate M (2-(4-bromo-1H-pyrazol-1-yl)-5-(trifluoromethyl)pyridine) (1.5 g, 5.136 mmol) was reacted with diphenylmethanimine (1.4 g, 7.71 mmol) following General Procedure 4 Method 1. Upon completion, the reaction mixture was concentrated to dryness. The crude product was re-suspended in MeOH, and stirred with NH₂OH·HCl (1.074 g, 15.41 mmol) and NaOAc (1.264 g, 15.41 mmol) at rt for 18 h. The volatile solvents were removed and the residue taken up in DCM and saturated aq. NaHCO₃ solutions. After separation the aqueous phase was extracted with DCM (4×), the combined organic phases were dried over (MgSO₄), evaporated to dryness. The residue was purified on silica gel using mixture of DCM and EtOAc, then DCM and MeOH as eluents to provide 1-(5-(trifluoromethyl)pyridin-2-yl)-1H-pyrazol-4-amine in 34.5% yield (0.405 g). ¹H NMR (401 MHz, CDCl₃) δ 8.59-8.55 (m, 1H), 8.05 (d, J=0.9 Hz, 1H), 7.95 (d, J=8.7 Hz, 1H), 7.91 (dd, J=8.8, 2.1 Hz, 1H), 7.43 (d, J=0.8 Hz, 1H), 3.16 (br s, 2H). LCMS R_{f} (min)=2.303, MS m/z=229.1 [M+H]⁺.

AcOH (0.23 mL, 4.08 mmol) was added to a solution of 1-(5-(Trifluoromethyl)pyridin-2-yl)-1H-pyrazol-4-amine (0.31 g, 1.358 mmol) and anisaldehyde (0.176 g, 1.49 mmol) was in DCE (7 mL), and the reaction mixture was stirred at rt for 18 h. NaBH(AcO)₃ (0.575 g, 2.72 mmol) was added in portions every hr, until most of the starting amine has been consumed as evidenced by TLC. The reaction mixture was diluted with DCM (10 mL), poured slowly into saturated aq. NaHCO₃ solution (20 mL). The aqueous phase was made alkaline (pH˜8) by addition of solid NaHCO₃. After separation, the aqueous phase was extracted with more DCM (3×). The combined organic phase was dried over MgSO₄, evaporated to dryness. The residue was purified on silica gel using mixtures of DCM and EtOAc as eluents to provide N-(4-methoxybenzyl)-1-(5-(trifluoromethyl)pyridin-2-yl)-1H-pyrazol-4-amine in 72.9% yield (0.345 g). ¹H NMR (401 MHz, CDCl₃) δ 8.58-8.56 (m, 1H), 7.97-7.92 (m, 2H), 7.89 (dd, J=8.8, 2.2 Hz, 1H), 7.43 (d, J=0.8 Hz, 1H), 7.32-7.26 (m, 2H), 6.90-6.83 (m, 2H), 4.15 (s, 2H), 3.78 (s, 3H), 3.34 (br s, 1H). ¹³C NMR (101 MHz, CDCl₃) δ 158.99, 153.70, 145.52, 145.48, 145.43, 145.39, 136.40, 135.46, 135.42, 134.64, 130.96, 128.98, 127.76, 125.07, 123.30, 122.97, 122.64, 122.37, 122.31, 119.67, 114.02, 111.14, 110.68, 55.24, 50.70. LCMS R_{f}(min)=3.218, MS m/z=349.1 [M+H]⁺.

N-(4-Methoxybenzyl)-1-(5-(trifluoromethyl)pyridin-2-yl)-1H-pyrazol-4-amine (0.345 g, 0.99 mmol) was reacted with 5-bromopicolinonitrile (0.272 g, 1.485 mmol) following General Procedure 4 Method 1. The crude product was purified on silica gel with mixtures of DCM and EtOAc as eluents to provide 5-((4-methoxybenzyl)(1-(5-(trifluoromethyl)pyridin-2-yl)-1H-pyrazol-4-yl)amino)picolinonitrile in 91.23% yield (0.407 g). ¹H NMR (401 MHz, CDCl₃) δ 8.63-8.61 (m, 1H), 8.59 (d, J=0.8 Hz, 1H), 8.28 (d, J=2.6 Hz, 1H), 8.08 (d, J=8.7 Hz, 1H), 8.03 (dd, J=8.7, 2.0 Hz, 1H), 7.73 (d, J=0.8 Hz, 1H), 7.43-7.40 (m, 1H), 7.19-7.16 (m, 2H), 7.12 (dd, J=8.8, 3.0 Hz, 1H), 6.87-6.84 (m, 2H), 4.88 (s, 2H), 3.75 (s, 3H). ¹³C NMR (101 MHz, CDCl₃) δ 159.25, 153.08, 146.03, 145.70, 145.66, 145.62, 145.57, 140.04, 137.70, 136.24, 136.21, 130.15, 128.97, 127.76, 127.54, 125.06, 124.73, 124.39, 124.06, 123.50, 122.03, 121.83, 119.40, 118.27, 114.53, 111.80, 56.25, 55.30. LCMS R_{f}(min)=4.121, MS m/z=451.2 [M+H]⁺.

5-((4-Methoxybenzyl)(1-(5-(trifluoromethyl)pyridin-2-yl)-1H-pyrazol-4-yl)amino)picolinonitrile (0.4 g, 0.888 mmol) was subjected to amidoxime formation in MeOH as per General Procedure 1 Method 2 to provide the desired N′-hydroxy-5-((4-methoxybenzyl)(1-(5-(trifluoromethyl)pyridin-2-yl)-1H-pyrazol-4-yl)amino)picolinimidamide after filtering and washing with H₂O, MeOH, and Et₂O (94.3% yield, 0.405 g). ¹H NMR (401 MHz, DMSO-d₆) δ 9.64 (s, 1H), 8.85 (m, 1H), 8.62 (d, J=0.8 Hz, 1H), 8.37 (dd, J=8.8, 2.0 Hz, 1H), 8.23 (d, J=2.5 Hz, 1H), 8.08 (d, J=8.7 Hz, 1H), 8.05 (d, J=0.8 Hz, 1H), 7.68 (d, J=8.8 Hz, 1H), 7.39 (dd, J=8.9, 2.9 Hz, 1H), 7.26 (m, 2H), 6.89 (m, 2H), 5.68 (s, 2H), 4.97 (s, 2H), 3.71 (s, 3H). LCMS R_{f} (min)=3.577, MS m/z=484.2 [M+H]⁺.

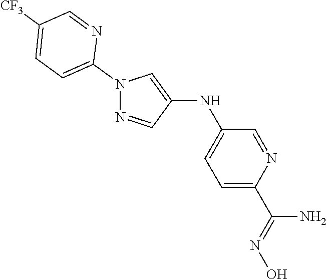 [SMILES: N/C(=N\O)c1ccc(Nc2cnn(-c3ccc(C(F)(F)F)cn3)c2)cn1]

N′-Hydroxy-5-((4-methoxybenzyl)(1-(5-(trifluoromethyl)pyridin-2-yl)-1H-pyrazol-4-yl)amino)picolinimidamide (0.4 g, 0.827 mmol) was subjected to PMB deprotection in TFA and anisole as per General Procedure 11 to furnish N′-hydroxy-5-((1-(5-(trifluoromethyl)pyridin-2-yl)-1H-pyrazol-4-yl)amino)picolinimidamide (81.5%, 243 mg) after purification with preparative HPLC. ¹H NMR (401 MHz, DMSO-d₆) δ 9.60 (s, 1H), 8.86 (s, 1H), 8.62 (s, 1H), 8.55 (s, 1H), 8.35 (dd, J=8.8, 2.2 Hz, 1H), 8.22 (d, J=2.6 Hz, 1H), 8.07 (d, J=8.7 Hz, 1H), 7.95 (s, 1H), 7.72 (d, J=8.7 Hz, 1H), 7.37 (dd, J=8.8, 2.8 Hz, 1H), 5.68 (s, 2H). ¹³C NMR (101 MHz, DMSO-d₆) δ 153.68, 150.05, 146.34, 141.79, 140.43, 138.03, 137.31, 134.95, 128.28, 125.56, 122.92, 122.60, 120.66, 120.58, 115.67, 111.98. LCMS R_{f}(min)=3.140, MS m/z=364.1 [M+H]⁺. HRMS (ESI) calcd for C₁₅H₁₃F₃N₇O⁺ [M+H]⁺ 364.1128, found 364.1145.

### 73. N′-Hydroxy-5-((1-(5-(trifluoromethyl)pyridin-2-yl)-1H-pyrazol-3-yl)amino)picolinimidamide (Scheme 70)

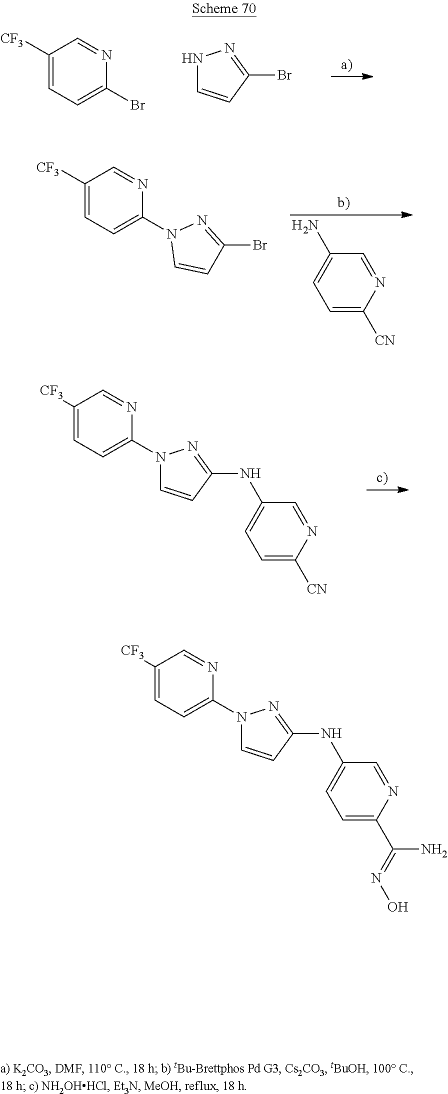 [SMILES: FC(F)(F)c1ccc(Br)nc1 ; Brc1cc[nH]n1 ; FC(F)(F)c1ccc(-n2ccc(Br)n2)nc1 ; N#Cc1ccc(N)cn1 ; N#Cc1ccc(Nc2ccn(-c3ccc(C(F)(F)F)cn3)n2)cn1 ; N/C(=N\O)c1ccc(Nc2ccn(-c3ccc(C(F)(F)F)cn3)n2)cn1]

Intermediate N: 2-(3-bromo-1H-pyrazol-1-yl)-5-(trifluoromethyl)pyridine 2-Bromo-5-(trifluoromethyl)pyridine (0.92 g, 4.08 mmol) was stirred with 3-bromo-1H-pyrazole (0.5 g, 3.4 mmol) in DMF (14 mL) at 110° C. for 18 h. The reaction mixture was cooled to rt, diluted with EtOAc and water. After separation the aqueous phase was extracted with EtOAc (4×), the combined organic phases were dried over (MgSO₄), evaporated to dryness. The residue was purified on silica gel using mixtures of petroleum spirits and DCM as eluents to provide 2-(3-bromo-1H-pyrazol-1-yl)-5-(trifluoromethyl)pyridine in 92.6% yield (0.92 g). ¹H NMR (401 MHz, CDCl₃) δ 8.67-8.65 (m, 1H), 8.50 (d, J=2.7 Hz, 1H), 8.07 (d, J=8.4 Hz, 1H), 8.04 (dd, J=8.9, 2.1 Hz, 1H), 6.52 (d, J=2.7 Hz, 1H). LCMS R_{f}(min)=4.168, MS m/z=292.0/294.0 [M+H]⁺.

Intermediate N (2-(3-bromo-1H-pyrazol-1-yl)-5-(trifluoromethyl)pyridine) (0.336 g, 1.15 mmol) was reacted with 5-aminonicotinonitrile (0.274 g, 2.3 mmol) following General Procedure 4 Method 2. Upon completion, the volatile solvents were removed and the residue taken up in EtOAc and water. After separation the aqueous phase was extracted with EtOAc (4×), the combined organic phases were dried over (MgSO₄), evaporated to dryness. The residue was purified on normal silica gel using mixture of DCM and EtOAc, then on reversed phase C18 silica gel using mixtures of CH₃CN and water as eluents to provide 5-((1-(5-(trifluoromethyl)pyridin-2-yl)-1H-pyrazol-3-yl)amino)picolinonitrile in 84.2% yield (0.32 g). ¹H NMR (401 MHz, CDCl₃) δ 8.70 (d, J=2.4 Hz, 1H), 8.67-8.63 (m, 1H), 8.55 (d, J=2.7 Hz, 1H), 8.12 (dd, J=8.6, 2.4 Hz, 1H), 8.04 (dd, J=8.7, 2.0 Hz, 1H), 7.96 (d, J=8.6 Hz, 1H), 7.66 (d, J=8.6 Hz, 1H), 6.69 (s, 1H), 6.21 (d, J=2.8 Hz, 1H). LCMS R_{f}(min)=3.079, MS m/z=331.1 [M+H]⁺.

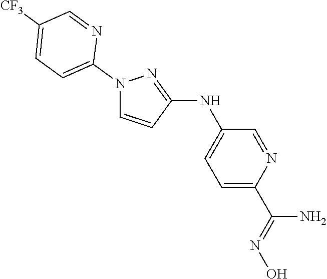 [SMILES: N/C(=N\O)c1ccc(Nc2ccn(-c3ccc(C(F)(F)F)cn3)n2)cn1]

5-((1-(5-(Trifluoromethyl)pyridin-2-yl)-1H-pyrazol-3-yl)amino)picolinonitrile (0.32 g, 0.968 mmol) was subjected to amidoxime formation in MeOH as per General Procedure 1 Method 2 to provide the desired N′-hydroxy-5-((1-(5-(trifluoromethyl)pyridin-2-yl)-1H-pyrazol-3-yl)amino)picolinimidamide after filtering and washing with H₂O, MeOH, and Et₂O (86.64% yield, 0.305 g). ¹H NMR NMR (401 MHz, DMSO) δ 9.68 (s, 1H), 9.57 (s, 1H), 8.82 (s, 1H), 8.79 (s, 1H), 8.57 (s, 1H), 8.33 (d, J=8.0 Hz, 1H), 8.08-7.94 (m, 2H), 7.80 (d, J=8.4 Hz, 1H), 6.28 (s, 1H), 5.75 (s, 2H). ¹³C NMR (101 MHz, DMSO) δ 154.24, 153.42, 150.11, 146.24, 141.55, 139.38, 137.41, 136.43, 128.96, 125.70, 123.36, 123.00, 121.93, 121.61, 120.19, 111.39, 100.70. LCMS R_{f}(min)=2.449, MS m/z=364.1 [M+H]⁺. HRMS (ESI) calcd for C₁₅H₁₃F₃N₇O⁺ [M+H]⁺ 364.1128, found 364.1136.

### 74. N′-Hydroxy-5-((1-(4-(trifluoromethyl)phenyl)-1H-pyrazol-3-yl)amino)picolinimidamide (Scheme 71)

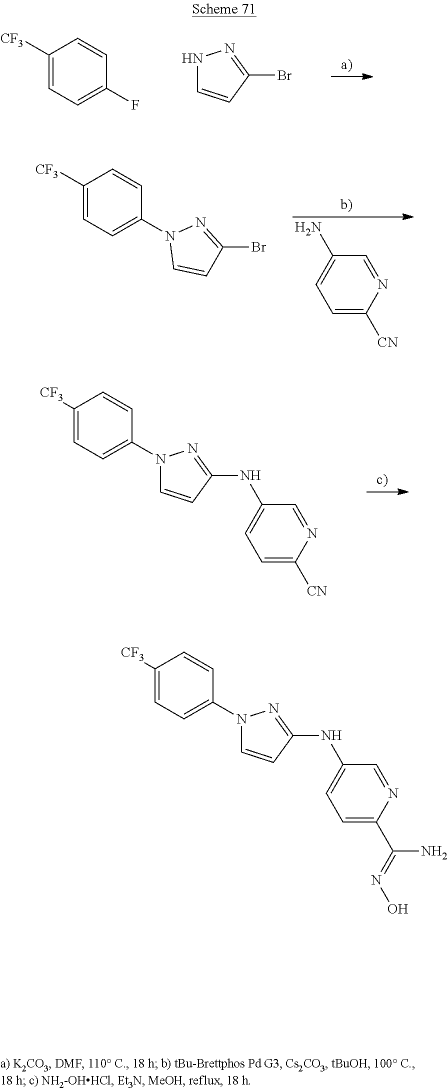 [SMILES: Fc1ccc(C(F)(F)F)cc1 ; Brc1cc[nH]n1 ; FC(F)(F)c1ccc(-n2ccc(Br)n2)cc1 ; N#Cc1ccc(N)cn1 ; N#Cc1ccc(Nc2ccn(-c3ccc(C(F)(F)F)cc3)n2)cn1 ; N/C(=N\O)c1ccc(Nc2ccn(-c3ccc(C(F)(F)F)cc3)n2)cn1]

Intermediate O: 3-bromo-1-(4-(trifluoromethyl)phenyl)-1H-pyrazole 1-Fluoro-4-(trifluoromethyl)benzene (1 g, 6.1 mmol) was reacted with 3-bromo-1H-pyrazole (0.895 g, 6.1 mmol) as per step a), Scheme 70 to provide 3-bromo-1-(4-(trifluoromethyl)phenyl)-1H-pyrazole in 84.56% yield (1.5 g). ¹H NMR (401 MHz, CDCl₃) δ 7.87 (d, J=2.6 Hz, 1H), 7.79 (d, J=8.5 Hz, 2H), 7.71 (d, J=8.6 Hz, 2H), 6.53 (d, J=2.6 Hz, 1H). LCMS R_{f}(min)=4.193, MS m/z=290.9/292.9 [M+H]⁺.

Intermediate O (3-bromo-1-(4-(trifluoromethyl)phenyl)-1H-pyrazole) (0.48 g, 1.65 mmol) was reacted with 5-aminonicotinonitrile (0.393 g, 3.3 mmol) as per step b), Scheme 70 to provide to provide 5-((1-(4-(trifluoromethyl)phenyl)-1H-pyrazol-3-yl)amino)picolinonitrile in 63.9% yield (0.347 g). ¹H NMR (401 MHz, MeOH-d₄) δ 8.71 (dd, J=2.7, 0.5 Hz, 1H), 8.34 (dd, J=8.7, 2.7 Hz, 1H), 8.31 (d, J=2.7 Hz, 1H), 7.97 (d, J=8.5 Hz, 2H), 7.79-7.75 (m, 3H), 6.25 (d, J=2.7 Hz, 1H). LCMS R_{f}(min)=3.062, MS m/z=330.0 [M+H]⁺.

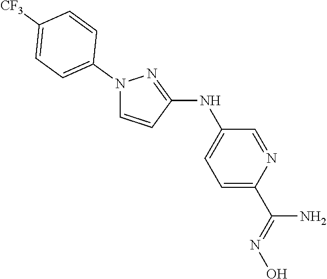 [SMILES: N/C(=N\O)c1ccc(Nc2ccn(-c3ccc(C(F)(F)F)cc3)n2)cn1]

5-((1-(4-(Trifluoromethyl)phenyl)-1H-pyrazol-3-yl)amino)picolinonitrile (0.345 g, 1.047 mmol) was subjected to amidoxime formation in MeOH as per General Procedure 1 Method 2 to provide the desired N′-hydroxy-5-((1-(4-(trifluoromethyl)phenyl)-1H-pyrazol-3-yl)amino)picolinimidamide after filtering and washing with H₂O, MeOH, and Et₂O (82.45% yield, 0.313 g). ¹H NMR (401 MHz, DMSO-d₆) δ 9.69 (s, 1H), 9.44 (s, 1H), 8.80 (s, 1H), 8.56 (d, J=2.1 Hz, 1H), 8.05-7.97 (m, 3H), 7.88-7.75 (m, 3H), 6.23 (d, J=2.1 Hz, 1H), 5.81 (s, 2H). ¹³C NMR (101 MHz, DMSO-d₆) δ 153.27, 150.28, 142.83, 140.98, 139.80, 136.21, 129.38, 127.26, 126.14, 125.26, 124.94, 123.44, 122.95, 120.27, 117.43, 99.42. LCMS R_{f}(min)=3.311, MS m/z=363.1 [M+H]⁺. HRMS (ESI) calcd for C₁₆H₁₄F₃N₆O⁺ [M+H]⁺ 363.1176, found 363.1168.

### 75. N′-Hydroxy-5-((3-(5-(trifluoromethyl)pyridin-2-yl)isoxazol-5-yl)amino)picolinimidamide (Scheme 72)

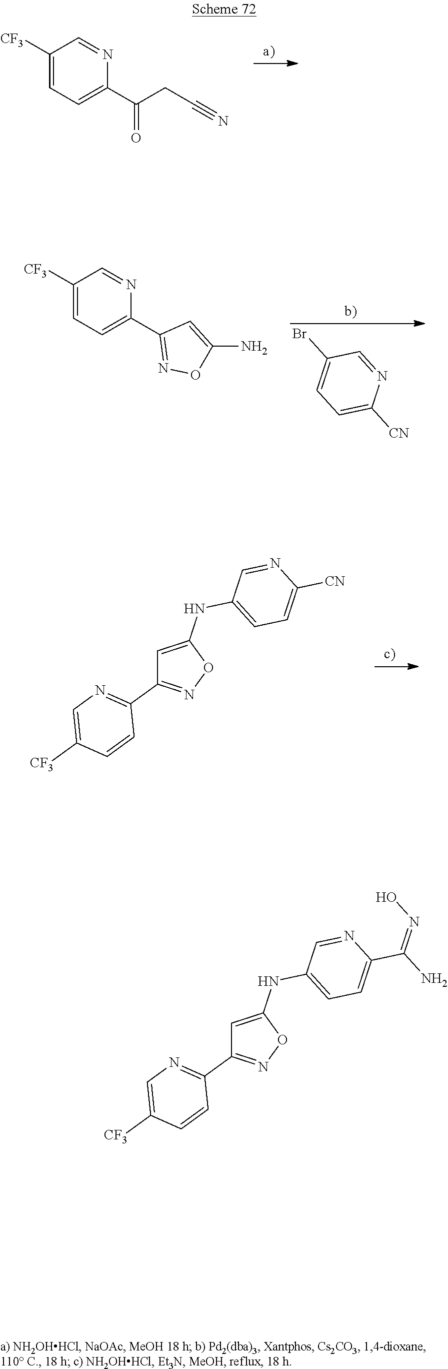 [SMILES: N#CCC(=O)c1ccc(C(F)(F)F)cn1 ; Nc1cc(-c2ccc(C(F)(F)F)cn2)no1 ; N#Cc1ccc(Br)cn1 ; N#Cc1ccc(Nc2cc(-c3ccc(C(F)(F)F)cn3)no2)cn1 ; N/C(=N/O)c1ccc(Nc2cc(-c3ccc(C(F)(F)F)cn3)no2)cn1]

NH₂OH·HCl (0.37 g, 5.3 mmol) and NaOAc (0.346 g, 5.3 mmol) were stirred in MeOH (9.0 mL) at room temperature for 1 h, and then 3-oxo-3-(5-(trifluoromethyl)pyridin-2-yl)propanenitrile (0.38 g, 1.77 mmol) was added to the mixture. The reaction mixture was stirred at rt for 18 h. Upon completion, the volatile solvents were removed and the residue taken up in EtOAc and saturated aq. NaHCO₃ solution. After separation the aqueous phase was extracted with EtOAc (4×), the combined organic phases were dried over (MgSO₄), evaporated to dryness. The residue was purified on silica gel using mixtures of EtOAc in DCM to provide 3-(5-(trifluoromethyl)pyridin-2-yl)isoxazol-5-amine in 92.95% yield (0.378 g). ¹H NMR (401 MHz, CDCl₃) δ 8.92-8.88 (m, 1H), 8.11 (d, J=8.3 Hz, 1H), 8.01-7.97 (m, 1H), 5.81 (s, 1H), 4.65 (br s, 2H). LCMS R_{f}(min)=3.434, MS m/z=230.0 [M+H]⁺.

3-(5-(Trifluoromethyl)pyridin-2-yl)isoxazol-5-amine (0.25 g, 1.09 mmol) was reacted with 5-bromopicolinonitrile (0.2 g, 1.09 mmol) following General Procedure 4 Method 1. The crude product was purified on silica gel with mixtures of DCM and EtOAc (0 to 40%) as eluents to provide 5-((3-(5-(trifluoromethyl)pyridin-2-yl)isoxazol-5-yl)amino)picolinonitrile in 23.8% yield (0.086 g). ¹H NMR (401 MHz, MeOH-d4) δ 9.02-8.97 (m, 1H), 8.56 (m, 1H), 8.26 (m, 1H), 8.22 (m, 1H), 7.87-7.83 (m, 2H), 6.48 (s, 1H). LCMS R_{f}(min)=3.678, MS m/z=332.1 [M+H]⁺.

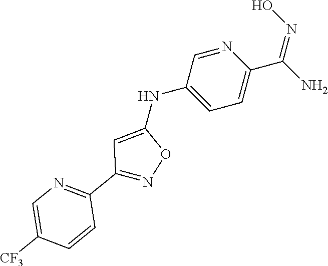 [SMILES: N/C(=N/O)c1ccc(Nc2cc(-c3ccc(C(F)(F)F)cn3)no2)cn1]

5-((3-(5-(Trifluoromethyl)pyridin-2-yl)isoxazol-5-yl)amino)picolinonitrile (0.086 g, 0.259 mmol) was subjected to amidoxime formation in MeOH as per General Procedure 1 Method 2 to provide the desired N′-hydroxy-5-((3-(5-(trifluoromethyl)pyridin-2-yl)isoxazol-5-yl)amino)picolinimidamide after filtering and washing with water and MeOH, then purified with preparative HPLC. (72.95% yield, 0.069 g). ¹H NMR (401 MHz, DMSO-d₆) δ 11.08 (s, 1H), 11.07-10.73 (br, 1H), 9.11 (d, J=2.2 Hz, 1H), 8.61 (d, J=2.6 Hz, 1H), 8.8-8.3 (br, 1H), 8.39 (dd, J=8.3, 2.3 Hz, 1H), 8.22 (d, J=8.3 Hz, 1H), 8.05 (d, J=8.8 Hz, 1H), 7.91 (dd, J=8.8, 2.7 Hz, 1H), 6.52 (s, 1H). LCMS R_{f}(min)=2.463, MS m/z=365.1 [M+H]⁺. HRMS (ESI) calcd for C₁₅H₁₂F₃N₆O₂⁺ [M+H]⁺ 365.0968, found 365.0979.

### 76. N′-Hydroxy-5-((1-methyl-3-(5-(trifluoromethyl)pyridin-2-yl)-1H-pyrazol-5-yl)amino)picolinimidamide (Scheme 73)

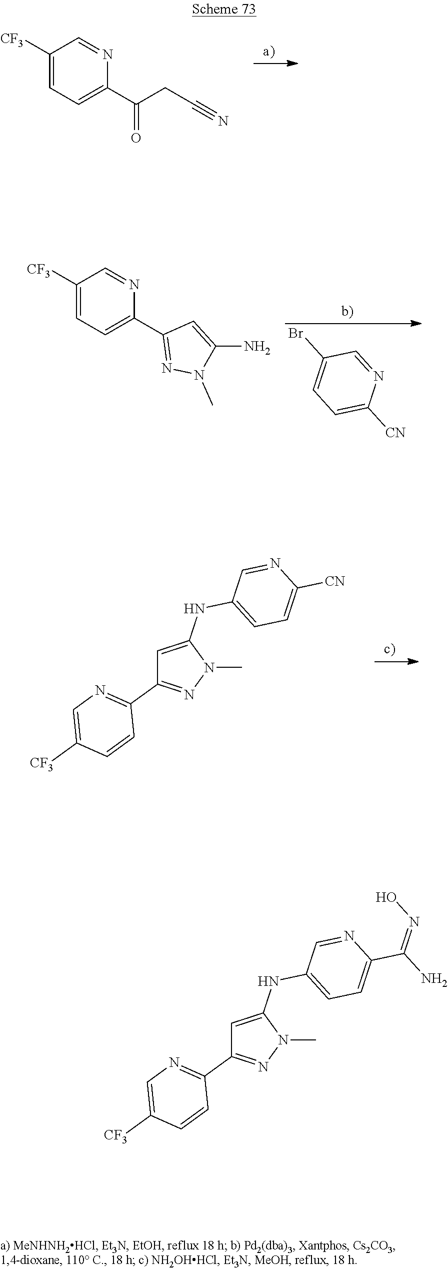 [SMILES: N#CCC(=O)c1ccc(C(F)(F)F)cn1 ; Cn1nc(-c2ccc(C(F)(F)F)cn2)cc1N ; N#Cc1ccc(Br)cn1 ; Cn1nc(-c2ccc(C(F)(F)F)cn2)cc1Nc1ccc(C#N)nc1 ; Cn1nc(-c2ccc(C(F)(F)F)cn2)cc1Nc1ccc(/C(N)=N\O)nc1]

3-Oxo-3-(5-(trifluoromethyl)pyridin-2-yl)propanenitrile (0.3 g, 1.4 mmol), MeNHNH₂·HCl (0.145 g, 1.75 mmol), and Et₃N (0.177 g, 1.75 mmol) were stirred in EtOH (7.0 mL) at room temperature for 0.5 h, then at reflux for 10 h. Upon completion, the volatile solvents were removed and the residue taken up in DCM and saturated aq. NaHCO₃. After separation the aqueous phase was extracted with DCM (4×), the combined organic phases were dried over (MgSO₄), evaporated to dryness. The residue was purified on silica gel using mixtures of petroleum spirit and EtOAc to provide 1-methyl-3-(5-(trifluoromethyl)pyridin-2-yl)-1H-pyrazol-5-amine in 31.7% yield (0.107 g). ¹H NMR (401 MHz, DMSO-d₆) δ 8.86 (s, 1H), 8.10 (dd, J=8.4, 2.0 Hz, 1H), 7.99 (d, J=8.5 Hz, 1H), 5.95 (s, 1H), 5.41 (s, 2H), 3.63 (s, 3H). LCMS R_{f}(min)=2.567, MS m/z=243.1 [M+H]⁺.

1-Methyl-3-(5-(trifluoromethyl)pyridin-2-yl)-1H-pyrazol-5-amine (0.098 g, 0.404 mmol) was reacted with 5-bromopicolinonitrile (0.074 g, 0.404 mmol) following General Procedure 4 Method 1. The crude product was purified on silica gel with mixtures of DCM and EtOAc (0 to 40%) as eluents to provide 5-((1-methyl-3-(5-(trifluoromethyl)pyridin-2-yl)-1H-pyrazol-5-yl)amino)picolinonitrile in 18.7% yield (0.026 g). ¹H NMR (401 MHz, MeOH-d4) δ 8.86 (s, 1H), 8.33 (dd, J=2.8, 0.5 Hz, 1H), 8.19-8.14 (m, 2H), 7.71 (dd, J=8.6, 0.5 Hz, 1H), 7.35 (dd, J=8.6, 2.8 Hz, 1H), 6.90 (s, 1H), 3.87 (s, 3H). LCMS R_{f}(min)=2.848, MS m/z=345.1 [M+H]⁺.

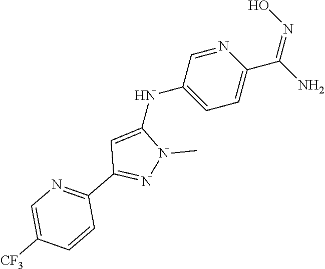 [SMILES: Cn1nc(-c2ccc(C(F)(F)F)cn2)cc1Nc1ccc(/C(N)=N\O)nc1]

5-((1-Methyl-3-(5-(trifluoromethyl)pyridin-2-yl)-1H-pyrazol-5-yl)amino)picolinonitrile (0.026 g, 0.075 mmol) was subjected to amidoxime formation in MeOH as per General Procedure 1 Method 2 to provide the desired N′-hydroxy-5-((1-methyl-3-(5-yl)amino)picolinimidamide after filtering and washing with water and MeOH. (49.13% yield, 0.014 g). ¹H NMR (401 MHz, DMSO-d₆) δ 10.95 (br s, 1H), 9.23 (s, 1H), 8.96-8.92 (m, 1H), 8.89-8.4 (br s, 2H), 8.40 (d, J=2.7 Hz, 1H), 8.23 (dd, J=8.4, 2.0 Hz, 1H), 8.13 (d, J=8.4 Hz, 1H), 7.94 (d, J=8.8 Hz, 1H), 7.42 (dd, J=8.8, 2.8 Hz, 1H), 6.82 (s, 1H), 3.82 (s, 3H). ¹³C NMR (101 MHz, DMSO-d₆) δ 155.72, 148.63, 146.62, 144.33, 140.79, 137.42, 134.80, 125.71, 124.19, 124.09, 123.87, 123.01, 120.44, 119.29, 96.77, 36.24. LCMS R_{f} (min)=2.367, MS m/z=378.1 [M+H]⁺. HRMS (ESI) calcd for C₁₆H₁₅F₃N₇O⁺ [M+H]⁺ 378.1285, found 378.1295.

### 77. N′-Hydroxy-5-((1-methyl-3-(4-(trifluoromethyl)phenyl)-1H-pyrazol-5-yl)amino)picolinimidamide (Scheme 74)

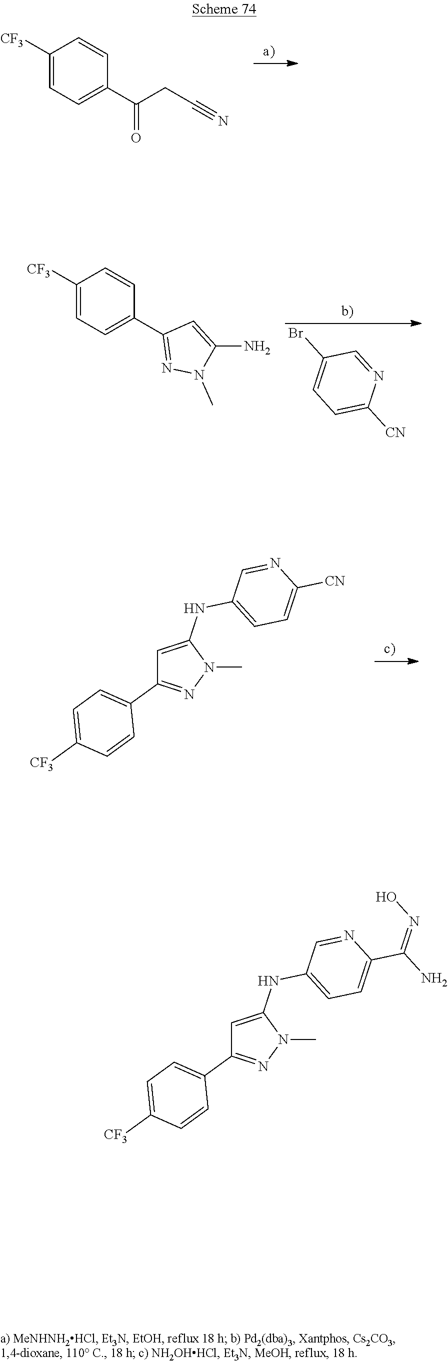 [SMILES: N#CCC(=O)c1ccc(C(F)(F)F)cc1 ; Cn1nc(-c2ccc(C(F)(F)F)cc2)cc1N ; N#Cc1ccc(Br)cn1 ; Cn1nc(-c2ccc(C(F)(F)F)cc2)cc1Nc1ccc(C#N)nc1 ; Cn1nc(-c2ccc(C(F)(F)F)cc2)cc1Nc1ccc(/C(N)=N\O)nc1]

3-Oxo-3-(4-(trifluoromethyl)phenyl)propanenitrile (0.48 g, 2.25 mmol) was converted to 1-methyl-3-(4-(trifluoromethyl)phenyl)-1H-pyrazol-5-amine as per step a), Scheme 73 (37% yield (0.201 g). ¹H NMR (401 MHz, CDCl₃) δ 7.81 (d, J=8.0 Hz, 2H), 7.60 (d, J=8.1 Hz, 2H), 5.89 (s, 1H), 3.72 (s, 3H), 3.59 (br s, 2H). LCMS R_{f}(min)=2.671, MS m/z=242.1 [M+H]⁺.

1-Methyl-3-(4-(trifluoromethyl)phenyl)-1H-pyrazol-5-amine (0.2 g, 0.83 mmol) was reacted with 5-bromopicolinonitrile (0.152 g, 0.83 mmol) following General Procedure 4 Method 1. The crude product was purified on silica gel with mixtures of DCM and EtOAc (0 to 40%) as eluents to provide 5-((1-methyl-3-(4-(trifluoromethyl)phenyl)-1H-pyrazol-5-yl)amino)picolinonitrile in 63.6% yield (0.181 g). ¹H NMR (401 MHz, DMSO-d₆) δ 9.21 (s, 1H), 8.37 (d, J=2.4 Hz, 1H), 8.03 (d, J=8.1 Hz, 2H), 7.82 (d, J=8.6 Hz, 1H), 7.75 (d, J=8.2 Hz, 2H), 7.35 (dd, J=8.7, 2.8 Hz, 1H), 6.87 (s, 1H), 3.77 (s, 3H). LCMS R_{f}(min)=2.888, MS m/z=344.1 [M+H]⁺.

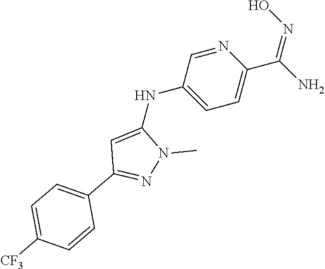 [SMILES: Cn1nc(-c2ccc(C(F)(F)F)cc2)cc1Nc1ccc(/C(N)=N\O)nc1]

5-((1-methyl-3-(4-(trifluoromethyl)phenyl)-1H-pyrazol-5-yl)amino)picolinonitrile (0.171 g, 0.498 mmol) was subjected to amidoxime formation in MeOH as per General Procedure 1 Method 2 to provide the desired N′-hydroxy-5-((1-methyl-3-(4-(trifluoromethyl)phenyl)-1H-pyrazol-5-yl)amino)picolinimidamide after filtering and washing with water and MeOH, then purified in preparative HPLC (72% yield (corrected for presence of 1 molar equivalent of DMSO), 0.163 g). ¹H NMR (401 MHz, DMSO-d₆) δ 9.64 (s, 1H), 8.59 (s, 1H), 8.26 (dd, J=2.7, 0.5 Hz, 1H), 8.02 (d, J=8.1 Hz, 2H), 7.76-7.70 (m, 3H), 7.35 (dd, J=8.8, 2.8 Hz, 1H), 6.74 (s, 1H), 5.70 (s, 2H), 3.77 (s, 3H). ¹³C NMR (101 MHz, DMSO-d₆) δ 149.96, 147.51, 142.00, 141.65, 141.39, 137.88, 135.58, 128.12, 127.81, 126.22, 126.03, 126.00, 125.96, 125.92, 125.78, 123.53, 122.00, 120.52, 94.21, 35.87. LCMS R_{f}(min)=2.450, MS m/z=377.1 [M+H]⁺. HRMS (ESI) calcd for C₁₇H₁₆F₃N₆O⁺ [M+H]⁺ 377.1332, found 377.1334.

### 78. 5-((3-(4-chlorophenyl)-1,2,4-oxadiazol-5-yl)amino)-N′-hydroxypicolinimidamide (Scheme 75)

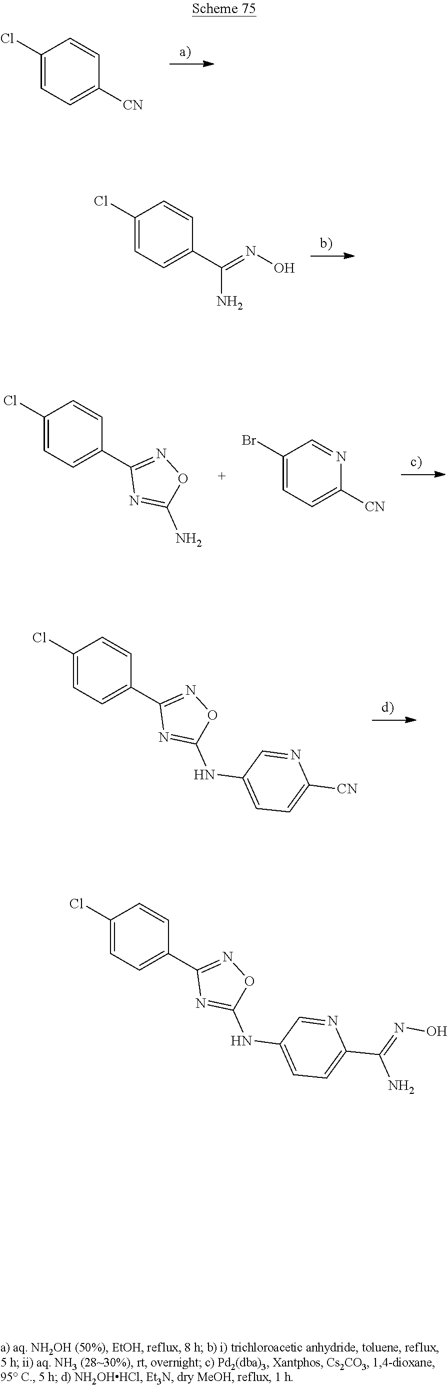 [SMILES: N#Cc1ccc(Cl)cc1 ; N/C(=N\O)c1ccc(Cl)cc1 ; Nc1nc(-c2ccc(Cl)cc2)no1 ; N#Cc1ccc(Br)cn1 ; N#Cc1ccc(Nc2nc(-c3ccc(Cl)cc3)no2)cn1 ; N/C(=N\O)c1ccc(Nc2nc(-c3ccc(Cl)cc3)no2)cn1]

4-Chlorobenzonitrile (2.0 g, 14.60 mmol) was reacted with NH₂OH (0.95 mL, 15.33 mmoL, aq. 50%) in EtOH (12 mL) for 8 h as per General Procedure 1 Method 1 to provide 4-chloro-N′-hydroxybenzimidamide in 100% yield (2.48 g).

4-Chloro-N′-hydroxybenzimidamide (2.46 g, 14.5 mmol) was reacted with trichloroacetic anhydride (2.91 mL, 15.92 mmol) in toluene (10 mL) for 8 h, followed by the treatment with NH₃ (50 mL, aq. 28-30%) as per General Procedure 3 to provide 3-(4-chlorophenyl)-1,2,4-oxadiazol-5-amine in 83% (2.34 g). ¹H NMR (401 MHz, DMSO-d₆) δ 8.00 (s, 2H), 7.87 (d, J=7.8 Hz, 2H), 7.57 (d, J=7.7 Hz, 2H). LCMS R_{f}(min)=2.626, MS m/z=196.0 [M+H]⁺.

3-(4-Chlorophenyl)-1,2,4-oxadiazol-5-amine (0.2 g, 1.03 mmol) was reacted with 5-bromopicolinonitrile (0.144 g, 0.79 mmol), Pd₂(dba)₃ (0.0434 g, 0.047 mmol), Xantphos (0.0366 g, 0.063 mmol) and Cs₂CO₃ (0.515 g, 1.58 mmol) in 1,4-dioxane (8 mL) per General Procedure 4 Method 1 to provide 5-((3-(4-chlorophenyl)-1,2,4-oxadiazol-5-yl)amino)picolinonitrile in 91% yield (0.215 g). ¹H NMR (401 MHz, DMSO-d₆) δ 8.83 (s, 1H), 8.36 (d, J=8.4 Hz, 1H), 8.03-8.02 (m, 3H), 7.63 (d, J=7.6 Hz, 2H). LCMS R_{f}(min)=3.959, MS m/z=298.0 [M+H]⁺.

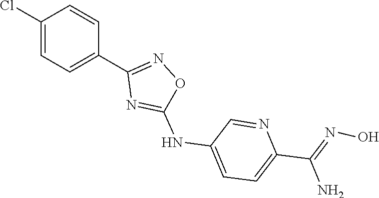 [SMILES: N/C(=N\O)c1ccc(Nc2nc(-c3ccc(Cl)cc3)no2)cn1]

5-((3-(4-Chlorophenyl)-1,2,4-oxadiazol-5-yl)amino)picolinonitrile (0.2 g, 0.67 mmol) was reacted with NH₂OH·HCl (0.14 g, 2.01 mmol) and Et₃N (0.28 mL, 2.01 mmol) in MeOH (8 mL) for 1 h as per General Procedure 1 Method 2 to provide 5-((3-(4-chlorophenyl)-1,2,4-oxadiazol-5-yl)amino)-N′-hydroxypicolinimidamide in 87% yield (0.196 g). ¹H NMR (401 MHz, DMSO-d₆) δ 11.53 (s, 1H), 9.87 (s, 1H), 8.82 (d, J=1.8 Hz, 1H), 8.13 (dd, J=8.7, 2.1 Hz, 1H), 8.01 (d, J=8.4 Hz, 2H), 7.90 (d, J=8.7 Hz, 1H), 7.62 (d, J=8.4 Hz, 2H), 5.84 (br s, 2H). ¹³C NMR (101 MHz, DMSO-d₆) δ 168.0, 166.7, 149.3, 144.5, 137.8, 136.1, 135.1, 129.3, 128.7, 125.6, 125.3, 119.9. LCMS R_{f}(min)=3.400, HRMS (ESI) calcd for C₁₄H₁₂ClN₆O₂⁺ [M+H]⁺ 331.0710, found 331.0715.

### 79. 5-((3-(4-chlorophenyl)-1,2,4-oxadiazol-5-yl)amino)-N′-hydroxypyrazine-2-carboximidamide (Scheme 76)

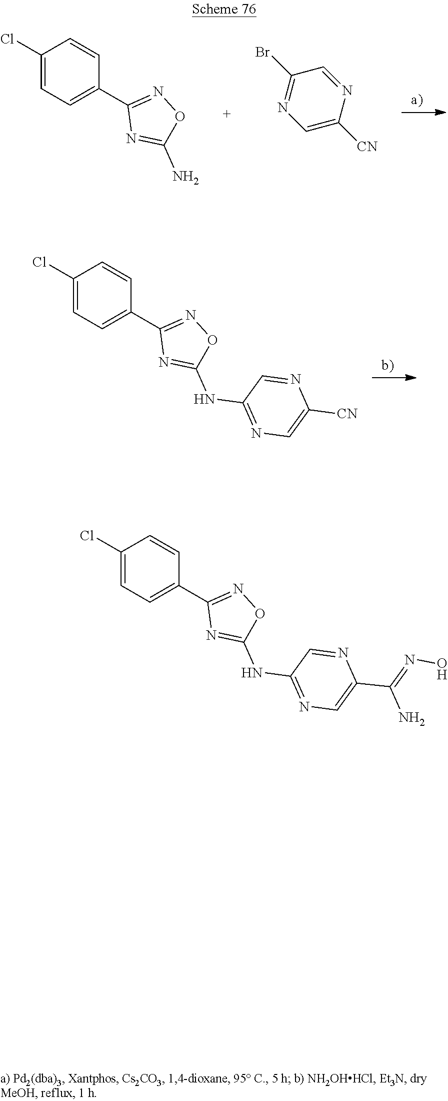 [SMILES: Nc1nc(-c2ccc(Cl)cc2)no1 ; N#Cc1cnc(Br)cn1 ; N#Cc1cnc(Nc2nc(-c3ccc(Cl)cc3)no2)cn1 ; N/C(=N\O)c1cnc(Nc2nc(-c3ccc(Cl)cc3)no2)cn1]

3-(4-Chlorophenyl)-1,2,4-oxadiazol-5-amine (0.2 g, 1.03 mmol) was reacted with 5-bromopyrazine-2-carbonitrile (0.144 g, 0.79 mmol), Pd₂(dba)₃ (0.0434 g, 0.047 mmol), Xantphos (0.0366 g, 0.063 mmol) and Cs₂CO₃ (0.515 g, 1.58 mmol) in 1,4-dioxane (8 mL) as per General Procedure 4 Method 1 to provide 5-((3-(4-chlorophenyl)-1,2,4-oxadiazol-5-yl)amino)pyrazine-2-carbonitrile in 93% yield (0.218 g). ¹H NMR (401 MHz, DMSO-d₆) δ 9.34 (s, 1H), 8.98 (s, 1H), 8.03 (d, J=8.0 Hz, 2H), 7.63 (d, J=8.0 Hz, 2H). LCMS R_{f}(min)=3.042, MS m/z=297.0 [M−H]⁻.

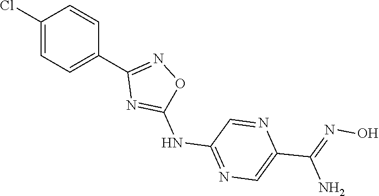 [SMILES: N/C(=N\O)c1cnc(Nc2nc(-c3ccc(Cl)cc3)no2)cn1]

(5-((3-(4-Chlorophenyl)-1,2,4-oxadiazol-5-yl)amino)pyrazine-2-carbonitrile (0.2 g, 0.67 mmol) was reacted with NH₂OH·HCl (0.14 g, 2.01 mmol) and Et₃N (0.28 mL, 2.01 mmol) in MeOH (8 mL) as per General Procedure 1 Method 2 to provide 5-((3-(4-chlorophenyl)-1,2,4-oxadiazol-5-yl)amino)-N′-hydroxypyrazine-2-carboximidamide in 82% yield (0.182 g). ¹H NMR (401 MHz, DMSO-d₆) δ 12.27 (br s, 1H), 10.05 (s, 1H), 9.27 (s, 1H), 8.82 (s, 1H), 8.03 (d, J=8.0 Hz, 2H), 7.64 (d, J=7.6 Hz, 2H), 5.91 (br s, 2H). ¹³C NMR (101 MHz, DMSO-d₆) δ 167.5, 166.6, 148.2, 147.4, 140.3, 139.4, 136.2, 132.4, 129.3, 128.7, 125.5. LCMS R_{f}(min)=2.682, HRMS (ESI) calcd for C₁₃H₁₁ClN₇O₂⁺ [M+H]⁺ 332.0663, found 332.0663.

### 80. 5-((3-(5-Chloropyridin-2-yl)-1,2,4-oxadiazol-5-yl)amino)-N′-hydroxypicolinimidamide (Scheme 77)

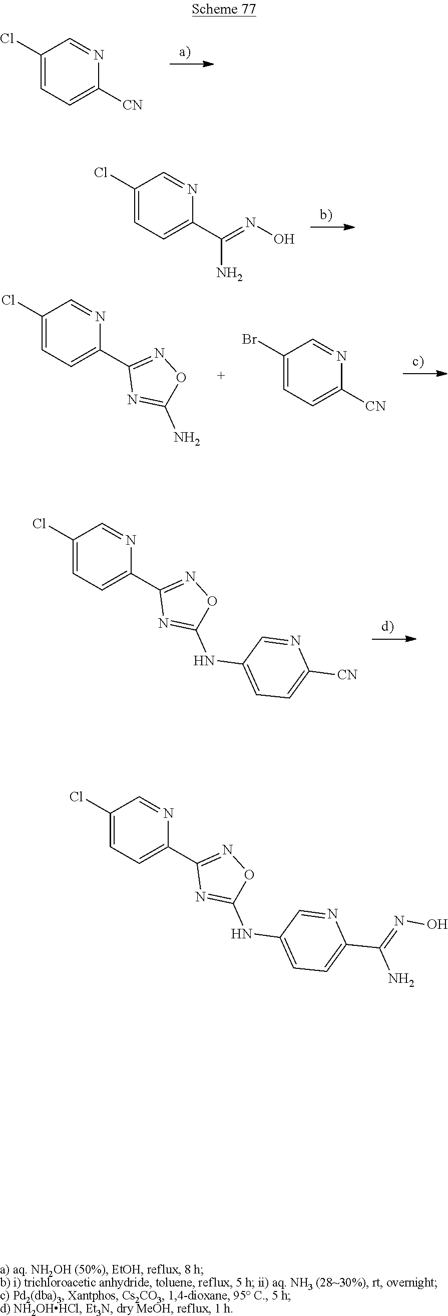

5-Chloropicolinonitrile (2.0 g, 14.49 mmol) was reacted with NH₂OH (0.93 mL, 15.22 mmoL, aq. 50%) in EtOH (12 mL) for 8 h as per General Procedure 1 Method 1 to provide 5-chloro-N′-hydroxypicolinimidamide in 100% yield (2.48 g).

5-Chloro-N′-hydroxypicolinimidamide (2.48 g, 14.49 mmol) was reacted with trichloroacetic anhydride (2.91 mL, 15.94 mmol) in toluene (10 mL) for 5 h, followed by the treatment with NH₃ (50 mL, aq. 28-30%) as per General Procedure 3 to provide 3-(5-chloropyridin-2-yl)-1,2,4-oxadiazol-5-amine in 75% yield (2.13 g). ¹H NMR (401 MHz, DMSO-d₆) δ 8.74 (s, 1H), 8.09 (m, 1H), 8.02 (s, 2H), 7.92 (d, J=8.4 Hz, 1H). LCMS R_{f}(min)=2.250, MS m/z=197.1 [M+H]⁺.

3-(5-Chloropyridin-2-yl)-1,2,4-oxadiazol-5-amine (0.3 g, 1.53 mmol) was reacted with 5-bromopicolinonitrile (0.215 g, 1.17 mmol), Pd₂(dba)₃ (0.0643 g, 0.070 mmol), Xantphos (0.0542 g, 0.094 mmol) and Cs₂CO₃ (0.762 g, 2.34 mmol) in 1,4-dioxane (12 mL) as per General Procedure 4 Method 1 to provide 5-((3-(5-chloropyridin-2-yl)-1,2,4-oxadiazol-5-yl)amino)picolinonitrile in 95% yield (0.33 g). ¹H NMR (401 MHz, DMSO-d₆) δ 8.76 (s, 1H), 8.62 (s, 1H), 8.28 (s, 1H), 8.09 (s, 2H), 7.86 (s, 1H), 7.40 (m, 1H). LCMS R_{f}(min)=2.685, MS m/z=299.0 [M+H]⁺.

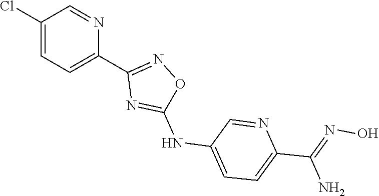 [SMILES: N/C(=N\O)c1ccc(Nc2nc(-c3ccc(Cl)cn3)no2)cn1]

5-((3-(5-Chloropyridin-2-yl)-1,2,4-oxadiazol-5-yl)amino)picolinonitrile (0.25 g, 0.84 mmol) was reacted with NH₂OH·HCl (0.174 g, 2.51 mmol) and Et₃N (0.35 mL, 2.51 mmol) in MeOH (8 mL) for 1 h as per General Procedure 1 Method 2 to provide 5-((3-(5-chloropyridin-2-yl)-1,2,4-oxadiazol-5-yl)amino)-N′-hydroxypicolinimidamide in 96% yield (0.268 g). ¹H NMR (401 MHz, DMSO-d₆) δ 11.56 (br s, 1H), 9.85 (s, 1H), 8.81 (s, 2H), 8.15-8.13 (m, 3H), 5.81 (br s, 2H). ¹³C NMR (101 MHz, DMSO-d₆) δ 168.2, 166.8, 149.2, 148.9, 144.7, 137.9, 137.4, 135.0, 133.2, 125.4, 124.5, 119.9. LCMS R_{f}(min)=3.015, HRMS (ESI) calcd for C₁₃H₁₁ClN₇O₂⁺ [M+H]⁺ 332.0663, found 332.0667.

### 81. 5-((3-(5-Chloropyridin-2-yl)-1,2,4-oxadiazol-5-yl)amino)-N′-hydroxypyrazine-2-carboximidamide (Scheme 78)

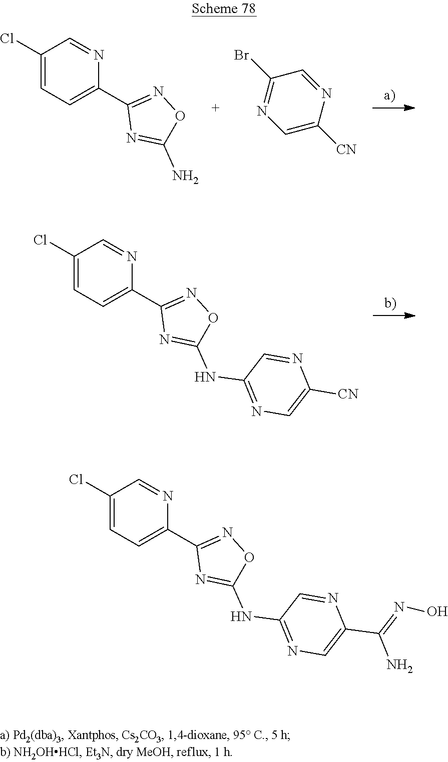 [SMILES: Nc1nc(-c2ccc(Cl)cn2)no1 ; N#Cc1cnc(Br)cn1 ; N#Cc1cnc(Nc2nc(-c3ccc(Cl)cn3)no2)cn1 ; N/C(=N\O)c1cnc(Nc2nc(-c3ccc(Cl)cn3)no2)cn1]

3-(5-Chloropyridin-2-yl)-1,2,4-oxadiazol-5-amine (0.3 g, 1.53 mmol) was reacted with 5-bromopyrazine-2-carbonitrile (0.216 g, 1.17 mmol), Pd₂(dba)₃ (0.0643 g, 0.07 mmol), Xantphos (0.0542 g, 0.094 mmol) and Cs₂CO₃ (0.762 g, 2.34 mmol) in 1,4-dioxane (12 mL) as per General Procedure 4 Method 1 to provide 5-((3-(5-chloropyridin-2-yl)-1,2,4-oxadiazol-5-yl)amino)pyrazine-2-carbonitrile 93% yield (0.326 g). ¹H NMR (401 MHz, DMSO-d₆) δ 8.76-8.67 (m, 2H), 8.50 (s, 1H), 8.06-8.03 (m, 3H). LCMS R_{f} (min)=2.649, MS m/z=300.0 [M−H]⁻.

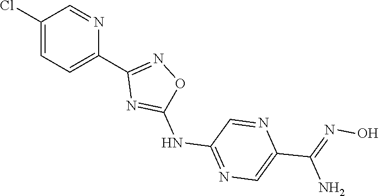 [SMILES: N/C(=N\O)c1cnc(Nc2nc(-c3ccc(Cl)cn3)no2)cn1]

5-((3-(5-Chloropyridin-2-yl)-1,2,4-oxadiazol-5-yl)amino)pyrazine-2-carbonitrile (0.25 g, 0.83 mmol) was reacted with NH₂OH·HCl (0.174 g, 2.50 mmol) and Et₃N (0.35 mL, 2.50 mmol) in MeOH (8 mL) for 1 h as per General Procedure 1 Method 2 to provide 5-((3-(5-chloropyridin-2-yl)-1,2,4-oxadiazol-5-yl)amino)-N′-hydroxypyrazine-2-carboximidamide in 70% yield (0.196 g). ¹H NMR (401 MHz, DMSO-d₆) δ 11.97 (br s, 1H), 10.05 (s, 1H), 9.27 (d, J=1.5 Hz, 1H), 8.83-8.82 (m, 2H), 8.13-8.18 (m, 2H), 5.91 (br s, 2H). ¹³C NMR (101 MHz, DMSO-d₆) δ 167.8, 166.8, 148.9, 148.2, 147.4, 144.5, 140.3, 139.4, 137.5, 133.3, 132.3, 124.5. LCMS R_{f} (min)=3.088, HRMS (ESI) calcd for C₁₂H₁₀ClN₈O₂⁺ [M+H]⁺ 333.0615, found 333.0614.

### 82. N′-hydroxy-5-((3-(4-(trifluoromethyl)phenyl)-1,2,4-oxadiazol-5-yl)amino)picolinimidamide (Scheme 79)

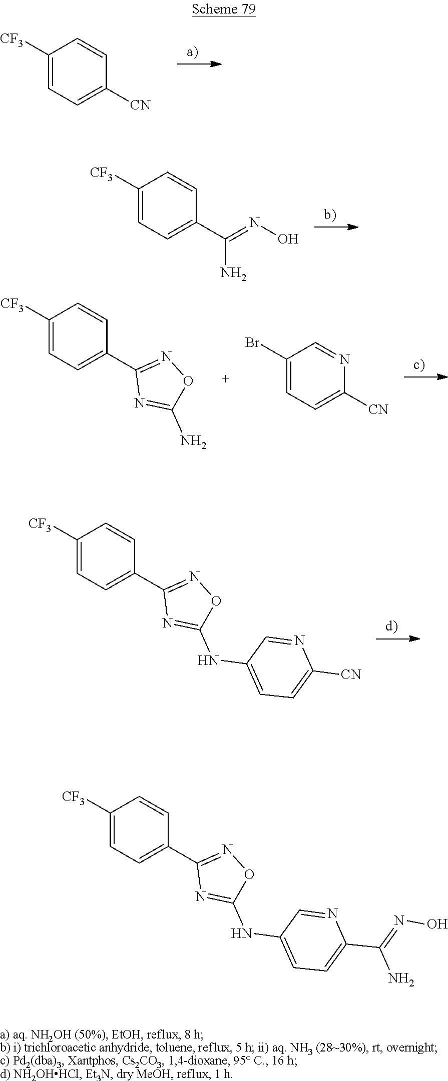

4-(Trifluoromethyl)benzonitrile (1.0 g, 5.84 mmol) was reacted with NH₂OH (0.37 mL, 6.13 mmol, aq. 50%) in EtOH (6 mL) for 8 h as per General Procedure 1 Method 1 to provide N′-hydroxy-4-(trifluoromethyl)benzimidamide in 100% yield (1.19 g).

N′-Hydroxy-4-(trifluoromethyl)benzimidamide (1.19 g, 5.84 mmol) was reacted with trichloroacetic anhydride (1.17 mL, 6.42 mmol) in toluene (6 mL) for 5 h, followed by the treatment with NH₃ (25 mL, aq. 28-30%) as per General Procedure 3 to provide 3-(4-(trifluoromethyl)phenyl)-1,2,4-oxadiazol-5-amine in 72% yield (0.96 g).

3-(4-(Trifluoromethyl)phenyl)-1,2,4-oxadiazol-5-amine (0.1 g, 0.44 mmol) was reacted with 5-bromopicolinonitrile (0.089 g, 0.48 mmol), Pd₂(dba)₃ (0.020 g, 0.022 mmol), Xantphos (0.025 g, 0.044 mmol) and Cs₂CO₃ (0.286 g, 0.88 mmol) in 1,4-dioxane (6 mL) as per General Procedure 4 Method 1 to provide 5-((3-(4-(trifluoromethyl)phenyl)-1,2,4-oxadiazol-5-yl)amino)picolinonitrile in 88% yield (0.128 g). ¹H NMR (401 MHz, MeOH-d₄) δ 8.92 (d, J=2.3 Hz, 1H), 8.51 (dd, J=8.6, 2.7 Hz, 1H), 8.30 (d, J=8.1 Hz, 2H), 7.94 (d, J=8.6 Hz, 1H), 8.30 (d, J=8.2 Hz, 2H). LCMS R_{f}(min)=3.946, MS m/z=330.0 [M−H]⁻.

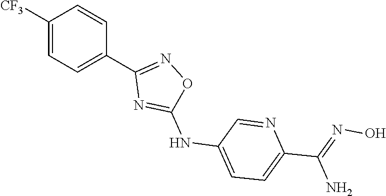 [SMILES: N/C(=N\O)c1ccc(Nc2nc(-c3ccc(C(F)(F)F)cc3)no2)cn1]

5-((3-(4-(Trifluoromethyl)phenyl)-1,2,4-oxadiazol-5-yl)amino)picolinonitrile (0.11 g, 0.33 mmol) was reacted with NH₂OH·HCl (0.184 g, 2.66 mmol) and Et₃N (0.37 mL, 2.66 mmol) in MeOH (5 mL) as per General Procedure 1 Method 2 to provide N′-hydroxy-5-((3-(4-(trifluoromethyl)phenyl)-1,2,4-oxadiazol-5-yl)amino)picolinimidamide in 38% yield (0.046 g). ¹H NMR (401 MHz, DMSO-d₆) δ 11.61 (s, 1H), 9.88 (s, 1H), 8.85 (d, J=2.2 Hz, 1H), 8.23 (d, J=8.1 Hz, 2H), 8.14 (dd, J=8.7, 2.5 Hz, 1H), 7.96-7.91 (m, 3H), 5.86 (br s, 2H). ¹³C NMR (101 MHz, DMSO-d₆) δ 168.3, 166.6, 149.3, 144.6, 137.9, 135.1, 131.4, 130.7, 127.8, 126.20, 126.17, 125.5, 125.3, 119.9. LCMS R_{f}(min)=2.630, HRMS (ESI) calcd for C₁₅H₁₂F₃N₆O₂⁺ [M+H]⁺ 365.0974, found 365.0979.

### 83. N′-Hydroxy-5-((3-(4-(trifluoromethyl)phenyl)-1,2,4-oxadiazol-5-yl)amino)pyrazine-2-carboximidamide (Scheme 80)

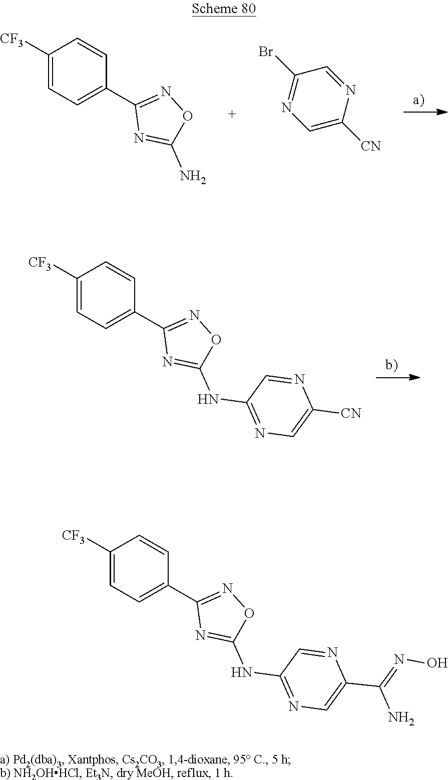

3-(4-(Trifluoromethyl)phenyl)-1,2,4-oxadiazol-5-amine (0.3 g, 1.31 mmol) was reacted with 5-bromopyrazine-2-carbonitrile (0.20 g, 1.09 mmol), Pd₂(dba)₃ (0.060 g, 0.065 mmol), Xantphos (0.050 g, 0.087 mmol) and Cs₂CO₃ (0.710 g, 2.18 mmol) in 1,4-dioxane (12 mL) as per General Procedure 4 Method 1 to provide 5-((3-(4-(trifluoromethyl)phenyl)-1,2,4-oxadiazol-5-yl)amino)pyrazine-2-carbonitrile in 98% yield (0.355 g). ¹H NMR (401 MHz, DMSO-d₆) δ 8.66 (d, J=1.4 Hz, 1H),), 8.46 (d, J=1.4 Hz, 1H),), 8.15 (d, J=8.0 Hz, 2H),), 7.86 (d, J=8.2 Hz, 2H). LCMS R_{f}(min)=3.082. MS m/z=331.0 [M−H]⁻.

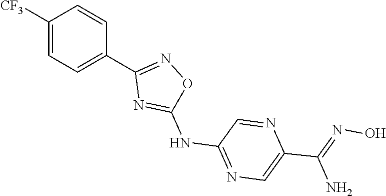 [SMILES: N/C(=N\O)c1cnc(Nc2nc(-c3ccc(C(F)(F)F)cc3)no2)cn1]

5-((3-(4-(Trifluoromethyl)phenyl)-1,2,4-oxadiazol-5-yl)amino)pyrazine-2-carbonitrile (0.30 g, 0.90 mmol) was reacted with NH₂OH·HCl (0.188 g, 2.71 mmol) and Et₃N (0.38 mL, 2.71 mmol) in MeOH (8 mL) for 1 h as per General Procedure 1 Method 2 to provide N′-hydroxy-5-((3-(4-(trifluoromethyl)phenyl)-1,2,4-oxadiazol-5-yl)amino)pyrazine-2-carboximidamide in 74% yield (0.242 g). ¹H NMR (401 MHz, DMSO-d₆) δ 12.29 (br s, 1H), 10.07 (s, 1H), 9.29 (d, J=1.4 Hz, 1H), 8.83 (d, J 1.4 Hz, 1H), 8.25 (d, J=8.1 Hz, 2H), 7.96 (d, J=8.3 Hz, 2H), 5.91 (br s, 2H). ¹³C NMR (101 MHz, DMSO-d₆) δ 168.2, 167.0, 148.7, 147.8, 140.9, 139.9, 132.8, 131.9, 131.5, 130.9, 128.3, 126.6, 125.7, 123.0. LCMS R_{f}(min)=2.761, HRMS (ESI) calcd for C₁₄H₁₁F₃N₇O₂⁺ [M+H]⁺ 366.0926, found 366.0939.

### 84. N′-Hydroxy-5-((3-(5-(trifluoromethyl)pyridin-2-yl)-1,2,4-oxadiazol-5-yl)amino)picolinimidamide (Scheme 81)

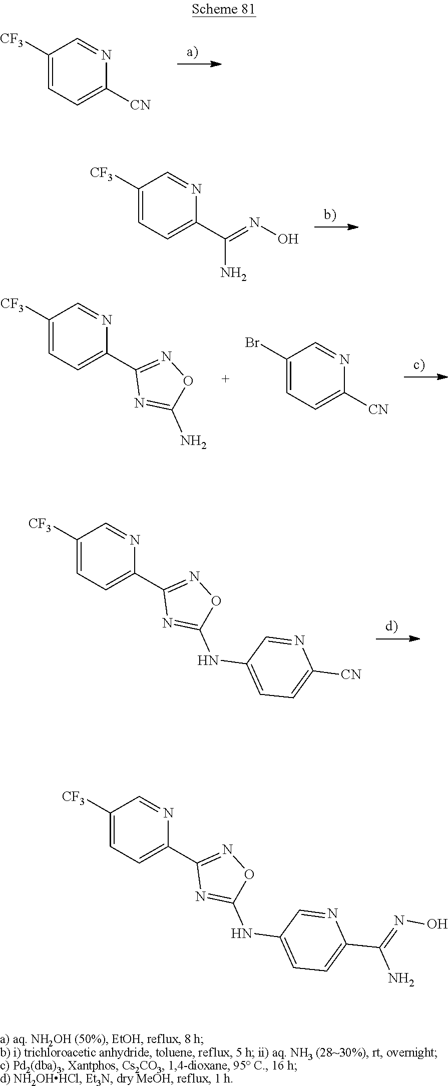

5-(Trifluoromethyl)picolinonitrile (2.0 g, 11.62 mmol) was reacted with NH₂OH (0.75 mL, 12.20 mmoL, aq. 50%) in EtOH (12 mL) for 8 h as per General Procedure 1 Method 1 to provide N′-hydroxy-5-(trifluoromethyl)picolinimidamide in 100% yield (2.38 g).

N′-Hydroxy-5-(trifluoromethyl)picolinimidamide (2.38 g, 11.62 mmol) was reacted with trichloroacetic anhydride (2.34 mL, 12.78 mmol) in toluene (6 mL) for 5 h, followed by the treatment of NH₃ (50 mL, aq. 28-30%) as per General Procedure 3 to provide 3-(5-(trifluoromethyl)pyridin-2-yl)-1,2,4-oxadiazol-5-amine in 63% yield (1.67 g). ¹H NMR (401 MHz, DMSO-d₆) δ 9.07 (s, 1H), 8.36 (s, 1H), 8.12-8.08 (m, 3H). LCMS R_{f}(min)=3.154, MS m/z=231.1 [M+H]⁺.

3-(5-(Trifluoromethyl)pyridin-2-yl)-1,2,4-oxadiazol-5-amine (0.31 g, 1.35 mmol) was reacted with 5-bromopicolinonitrile (0.176 g, 0.96 mmol), Pd₂(dba)₃ (0.053 g, 0.058 mmol), Xantphos (0.044 g, 0.077 mmol) and Cs₂CO₃ (0.626 g, 1.92 mmol) in 1,4-dioxane (10 mL) as per General Procedure 4 Method 1 to provide 5-((3-(5-(trifluoromethyl)pyridin-2-yl)-1,2,4-oxadiazol-5-yl)amino)picolinonitrile in 96% yield (0.308 g). ¹H NMR (401 MHz, DMSO-d₆) δ 9.12 (s, 1H), 8.83 (s, 1H), 8.40-8.34 (m, 4H), 8.02 (s, 1H). LCMS R_{f} (min)=3.676, MS m/z=333.1 [M+H]⁺.

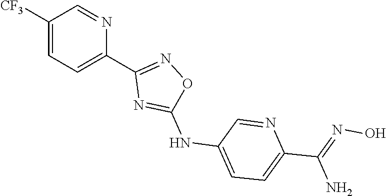 [SMILES: N/C(=N\O)c1ccc(Nc2nc(-c3ccc(C(F)(F)F)cn3)no2)cn1]

5-((3-(5-(Trifluoromethyl)pyridin-2-yl)-1,2,4-oxadiazol-5-yl)amino)picolinonitrile (0.10 g, 0.30 mmol) was reacted with NH₂OH·HCl (0.063 mg, 0.90 mmol) and Et₃N (0.13 mL, 0.90 mmol) in MeOH (5 mL) for 1 h as per General Procedure 1 Method 2 to provide N′-hydroxy-5-((3-(5-(trifluoromethyl)pyridin-2-yl)-1,2,4-oxadiazol-5-yl)amino)picolinimidamide in 81% yield (0.088 g). ¹H NMR (401 MHz, DMSO-d₆) δ 9.87 (s, 1H), 9.15 (s, 1H), 8.82 (d, J=2.3 Hz, 1H), 8.44 (dd, J=8.3, 1.9 Hz, 1H), 8.31 (d, J=8.2 Hz, 1H), 8.14 (dd, J=8.8, 2.6 Hz, 1H), 7.91 (d, J=8.7 Hz, 1H), 5.81 (br s, 2H). ¹³C NMR (101 MHz, DMSO-d₆) δ 168.6, 166.9, 149.9, 149.5, 147.1, 144.9, 138.2, 137.9, 135.5, 135.2, 127.0, 126.6, 125.7, 124.9, 123.5, 120.1. LCMS R_{f}(min)=3.158, HRMS (ESI) calcd for C₁₄H₁₁F₃N₇O₂⁺ [M+H]⁺ 366.0926, found 366.0933.

### 85. N′-hydroxy-5-((3-(5-(trifluoromethyl)pyridin-2-yl)-1,2,4-oxadiazol-5-yl)amino)pyrazine-2-carboximidamide (Scheme 82)

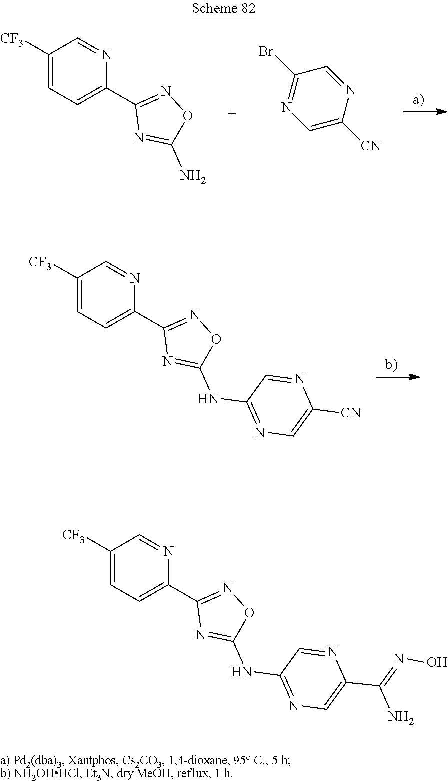

3-(5-(Trifluoromethyl)pyridin-2-yl)-1,2,4-oxadiazol-5-amine (0.3 g, 1.30 mmol) was reacted with 5-bromopyrazine-2-carbonitrile (0.171 g, 0.93 mmol), Pd₂(dba)₃ (0.051 g, 0.056 mmol), Xantphos (0.043 g, 0.074 mmol) and Cs₂CO₃ (0.606 g, 1.86 mmol) in 1,4-dioxane (12 mL) as per General Procedure 4 Method 1 to provide 5-((3-(5-(trifluoromethyl)pyridin-2-yl)-1,2,4-oxadiazol-5-yl)amino)pyrazine-2-carbonitrile in 98% yield (0.305 g). ¹H NMR (401 MHz, DMSO-d₆) δ 9.08 (s, 1H), 8.21-8.61 (m, 5H). LCMS R_{f}(min)=3.649, MS m/z=334.1 [M+H]⁺.

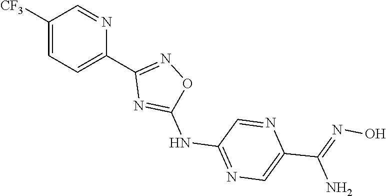 [SMILES: N/C(=N\O)c1cnc(Nc2nc(-c3ccc(C(F)(F)F)cn3)no2)cn1]

5-((3-(5-(Trifluoromethyl)pyridin-2-yl)-1,2,4-oxadiazol-5-yl)amino)pyrazine-2-carbonitrile (0.15 g, 0.45 mmol) was reacted with NH₂OH·HCl (0.094 g, 1.35 mmol) and Et₃N (0.19 mL, 1.35 mmol) in MeOH (6 mL) for 1 h as per General procedure 1 Method 2 to provide N′-hydroxy-5-((3-(5-(trifluoromethyl)pyridin-2-yl)-1,2,4-oxadiazol-5-yl)amino)pyrazine-2-carboximidamide in 53% yield (0.088 g). ¹H NMR (401 MHz, DMSO-d₆) δ 12.33 (br s, 1H), 10.07 (s, 1H), 9.28 (d, J=1.5 Hz, 1H), 8.83 (d, J=1.5 Hz, 1H), 8.46 (dd, J=8.3, 1.9 Hz, 1H), 8.33 (d, J=8.2 Hz, 1H), 5.91 (br s, 2H). ¹³C NMR (101 MHz, DMSO-d₆) δ 168.0, 166.7, 149.6, 148.2, 147.3, 147.0, 140.5, 139.4, 135.4, 132.4, 126.8, 126.5, 124.8, 123.4, 122.1. LCMS R_{f}(min)=2.456, HRMS (ESI) calcd for C₁₃H₁₀F₃N₈O₂+[M+H]⁺ 367.0879, found 367.0889.

### 86. N′-Hydroxy-5-((3-(4-(trifluoromethoxy)phenyl)-1,2,4-oxadiazol-5-yl)amino)picolinimidamide (Scheme 83)

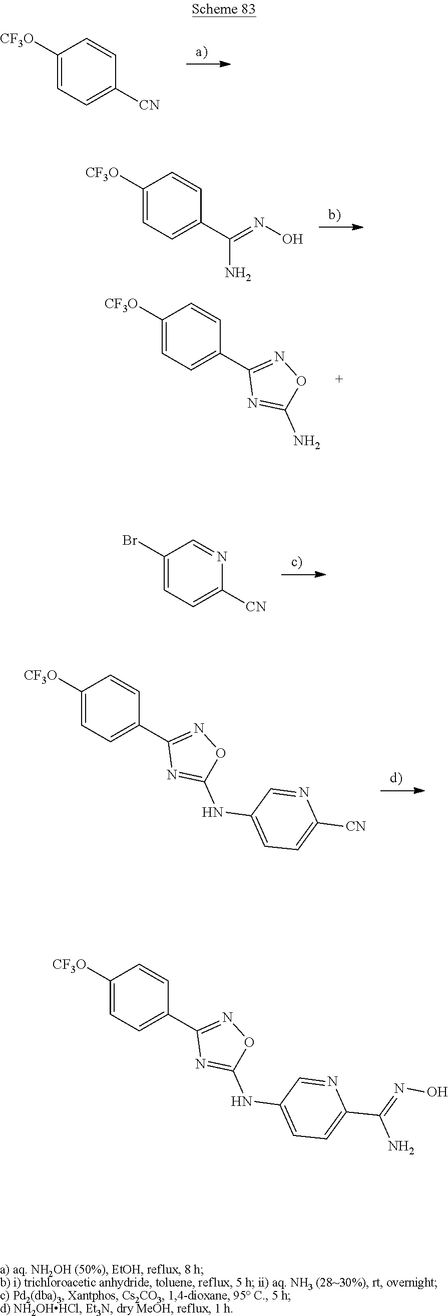

4-(Trifluoromethoxy)benzonitrile (2.0 g, 10.69 mmol) was reacted with NH₂OH (0.69 mL, 11.23 mmoL, aq. 50%) in EtOH (12 mL) for 8 h as per General Procedure 1 Method 1 to provide N′-hydroxy-4-(trifluoromethoxy)benzimidamide in 100% yield (2.35 g).

N′-Hydroxy-4-(trifluoromethoxy)benzimidamide (2.35 g, 10.69 mmol) was reacted with trichloroacetic anhydride (2.15 mL, 11.76 mmol) in toluene (10 mL) for 5 h, followed by the treatment with NH₃ (50 mL, aq. 28-30%) as per General Procedure 3 to provide 3-(4-(trifluoromethoxy)phenyl)-1,2,4-oxadiazol-5-amine in 78% yield (2.11 g). ¹H NMR (401 MHz, DMSO-d₆) δ 8.06-7.94 (m, 4H), 7.50 (d, J=8.0 Hz, 2H). LCMS R_{f}(min)=2.774, MS m/z=246.1 [M+H]⁺.

3-(4-(Trifluoromethoxy)phenyl)-1,2,4-oxadiazol-5-amine (0.30 g, 1.22 mmol) was reacted with 5-bromopicolinonitrile (0.171 g, 0.94 mmol), Pd₂(dba)₃ (0.052 g, 0.056 mmol), Xantphos (0.044 g, 0.075 mmol) and Cs₂CO₃ (0.612 g, 1.88 mmol) in 1,4-dioxane (12 mL) as per General Procedure 4 Method 1 to provide 5-((3-(4-(trifluoromethoxy)phenyl)-1,2,4-oxadiazol-5-yl)amino)picolinonitrile in 64% yield (0.21 g). ¹H NMR (401 MHz, DMSO-d₆) δ 8.75 (s, 1H), 8.32 (d, J=7.7 Hz, 1H), 8.12 (d, J=7.8 Hz, 2H), 7.95 (d, J=8.4 Hz, 1H), 7.54 (d, J=7.3 Hz, 2H). LCMS R_{f} (min)=3.145, MS m/z=348.0 [M+H]⁺.

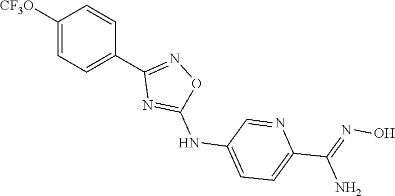

5-((3-(4-(Trifluoromethoxy)phenyl)-1,2,4-oxadiazol-5-yl)amino)picolinonitrile (0.15 g, 0.43 mmol) was reacted with NH₂OH·HCl (0.09 g, 1.30 mmol) and Et₃N (0.18 mL, 1.30 mmol) in MeOH (5 mL) for 1 h as per General Procedure 1 Method 2 to provide N′-hydroxy-5-((3-(4-(trifluoromethoxy)phenyl)-1,2,4-oxadiazol-5-yl)amino)picolinimidamide in 95% yield (0.155 g). ¹H NMR (401 MHz, DMSO-d₆) δ 11.53 (br s, 1H), 9.85 (s, 1H), 8.84 (s, 1H), 8.15-8.13 (m, 3H), 7.91 (d, J=8.7, 1H), 7.56 (d, J=8.2 Hz, 2H), 5.81 (br s, 2H). ¹³C NMR (101 MHz, DMSO-d₆) δ 168.1, 166.5, 150.3, 149.2, 144.6, 137.9, 135.1, 129.1, 126.0, 125.4, 121.6, 119.8. LCMS R_{f}(min)=3.535, HRMS (ESI) calcd for C₁₅H₁₂F₃N₆O₃⁺ [M+H]⁺ 381.0917, found 381.0930.

### 87. N′-Hydroxy-5-((3-(4-(trifluoromethoxy)phenyl)-1,2,4-oxadiazol-5-yl)amino)pyrazine-2-carboximidamide (Scheme 84)

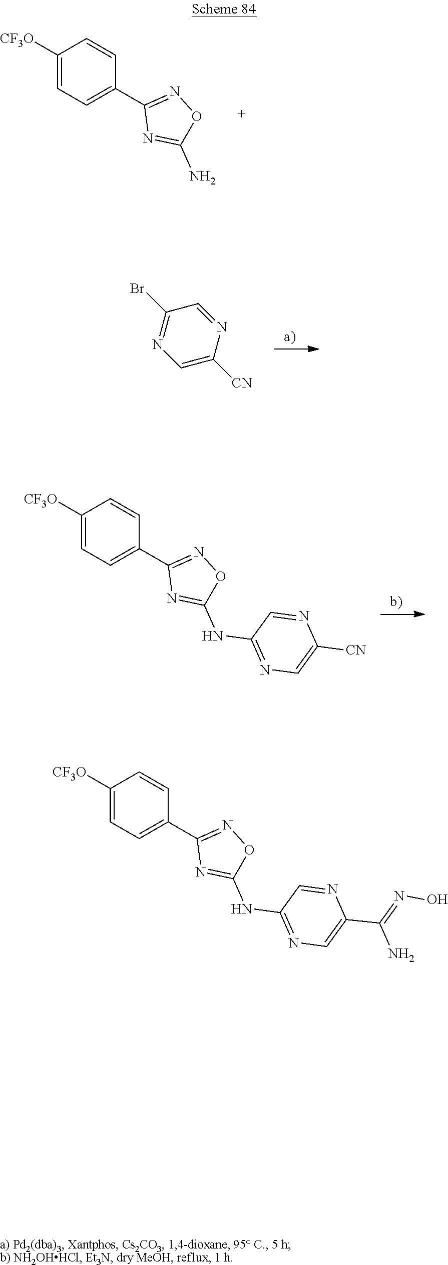

3-(4-(Trifluoromethoxy)phenyl)-1,2,4-oxadiazol-5-amine (0.3 g, 1.22 mmol) was reacted with 5-bromopyrazine-2-carbonitrile (0.187 g, 1.02 mmol), Pd₂(dba)₃ (0.056 g, 0.061 mmol), Xantphos (0.047 g, 0.082 mmol) and Cs₂CO₃ (0.665 g, 2.04 mmol) in 1,4-dioxane (12 mL) as per General Procedure 4 Method 1 to provide 5-((3-(4-(trifluoromethoxy)phenyl)-1,2,4-oxadiazol-5-yl)amino)pyrazine-2-carbonitrile in 79% yield (0.283 g). ¹H NMR (401 MHz, DMSO-d₆) δ 9.26 (s, 1H), 8.91 (s, 1H), 8.15 (d, J=6.7 Hz, 2H), 7.57 (d, J=7.3 Hz, 2H), LCMS R_{f}(min)=3.129, MS m/z=347.0 [M−H]⁻.

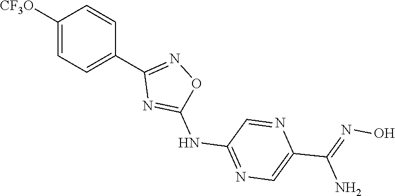

5-((3-(4-(Trifluoromethoxy)phenyl)-1,2,4-oxadiazol-5-yl)amino)pyrazine-2-carbonitrile (0.21 g, 0.60 mmol) was reacted with NH₂OH·HCl (0.126 g, 1.81 mmol) and Et₃N (0.25 mL, 1.81 mmol) in MeOH (7 mL) for 1 h As per General Procedure 1 Method 2 to provide N′-hydroxy-5-((3-(4-(trifluoromethoxy)phenyl)-1,2,4-oxadiazol-5-yl)amino)pyrazine-2-carboximidamide in 90% yield (0.206 g). ¹H NMR (401 MHz, DMSO-d₆) δ 12.27 (br s, 1H), 10.06 (s, 1H), 9.27 (d, J=1.1 Hz, 1H), 8.82 (d, J=1.1 Hz, 1H), 8.15 (d, J=8.8, 2H), 7.57 (d, J=8.2 Hz, 2H), 5.90 (br s, 2H). ¹³C NMR (101 MHz, DMSO-d₆) δ 167.6, 166.5, 150.4, 148.2, 147.5, 140.4, 139.4, 132.3, 129.2, 125.8, 121.6, 121.3, 118.7. LCMS R_{f} (min)=3.684, HRMS (ESI) calcd for C₁₄H₁₁F₃N₇O₃⁺ [M+H]⁺ 382.0875, found 382.0882.

### 88. N′-hydroxy-5-((3-(5-(trifluoromethoxy)pyridin-2-yl)-1,2,4-oxadiazol-5-yl)amino)picolinimidamide (Scheme 85)

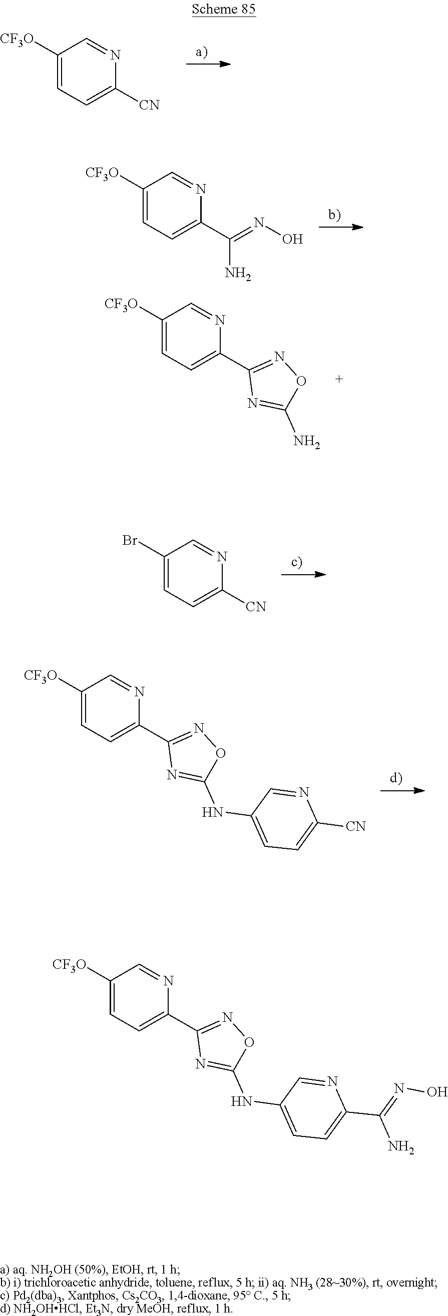

5-(Trifluoromethoxy)picolinonitrile (1.0 g, 5.32 mmol) was reacted with NH₂OH (0.36 mL, 5.85 mmoL, aq. 50%) in EtOH (6 mL) for 1 h as per General Procedure 1 Method 1 to provide N′-hydroxy-5-(trifluoromethoxy)picolinimidamide in 100% yield (1.18 g). ¹H NMR (401 MHz, DMSO-d₆) δ 10.08 (s, 1H), 8.64 (d, J=2.7 Hz, 1H), 7.99 (dd, J=8.9, 0.6 Hz, 1H), 7.93-7.89 (m, 1H), 5.89 (br s, 2H). LCMS R_{f}(min)=2.528, MS m/z=222.1 [M+H]⁺.

N′-Hydroxy-5-(trifluoromethoxy)picolinimidamide (1.16 g, 5.32 mmol) was reacted with trichloroacetic anhydride (1.05 mL, 5.77 mmol) in toluene (5 mL) for 5 h, followed by the treatment with NH₃ (45 mL, aq. 28-30%) as per General Procedure 3 to provide 3-(5-(trifluoromethoxy)pyridin-2-yl)-1,2,4-oxadiazol-5-amine in 93% yield (1.16 g). ¹H NMR (401 MHz, DMSO-d₆) δ 8.78 (s, 1H), 8.06-8.05 (m, 4H). LCMS R_{f}(min)=3.204, MS m/z=247.1 [M+H]⁺.

3-(5-(Trifluoromethoxy)pyridin-2-yl)-1,2,4-oxadiazol-5-amine (0.20 g, 0.81 mmol) was reacted with 5-bromopicolinonitrile (0.124 g, 0.68 mmol), Pd₂(dba)₃ (0.038 g, 0.041 mmol), Xantphos (0.032 g, 0.054 mmol) and Cs₂CO₃ (0.443 g, 1.36 mmol) in 1,4-dioxane (8 mL) as per General Procedure 4 Method 1 to provide 5-((3-(5-(trifluoromethoxy)pyridin-2-yl)-1,2,4-oxadiazol-5-yl)amino)picolinonitrile in 95% yield (0.268 g). ¹H NMR (401 MHz, DMSO-d₆) δ 8.89 (s, 1H), 8.86 (s, 1H), 8.38 (d, J=7.6 Hz, 1H), 8.26 (d, J=8.3 Hz, 1H), 8.08-8.13 (m, 2H). LCMS R_{f}(min)=2.841, MS m/z=349.0 [M+H]⁺.

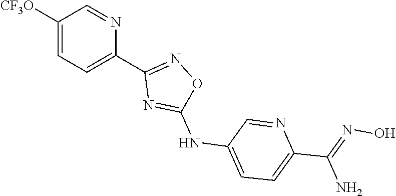

5-((3-(5-(Trifluoromethoxy)pyridin-2-yl)-1,2,4-oxadiazol-5-yl)amino)picolinonitrile (0.20 g, 0.574 mmol) was reacted with NH₂OH·HCl (0.12 g, 1.72 mmol), Et₃N (0.24 mL, 1.72 mmol) in MeOH (6 mL), reflux for 1 as per General Procedure 1 Method 2 to provide N′-hydroxy-5-((3-(5-(trifluoromethoxy)pyridin-2-yl)-1,2,4-oxadiazol-5-yl)amino)picolinimidamide in 71% yield (0.156 g). ¹H NMR (401 MHz, DMSO-d₆) δ 11.42 (s, 1H), 9.85 (s, 1H), 8.85 (s, 1H), 8.82 (s, 1H), 8.25 (d, J=8.6 Hz, 1H), 8.15-8.10 (m, 2H), 7.91 (d, J=8.7 Hz, 1H), 5.81 (br s, 2H). ¹³C NMR (101 MHz, DMSO-d₆) δ 168.3, 166.6, 149.2, 146.4, 145.2, 144.7, 143.2, 137.9, 135.0, 130.0, 125.4, 124.7, 121.3, 119.9, 118.7. LCMS R_{f}(min)=3.165, HRMS (ESI) calcd for C₁₄H₁₁F₃N₇O₃⁺ [M+H]⁺ 382.0875, found 382.0885.

### 89. N′-hydroxy-5-((3-(4-(trifluoromethyl)phenyl)isoxazol-5-yl)amino)picolinimidamide (Scheme 86)

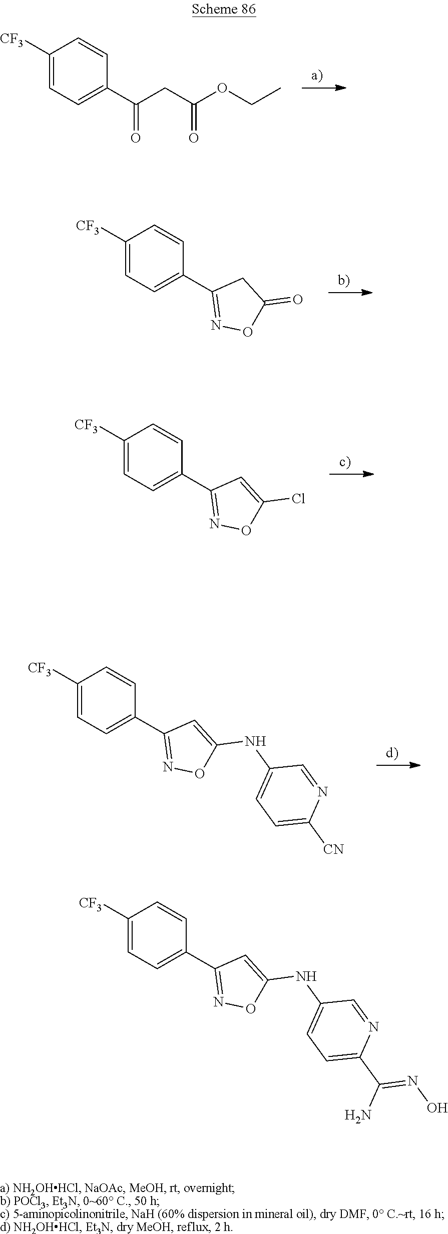

Ethyl 3-oxo-3-(4-(trifluoromethyl)phenyl)propanoate (1.0 g, 3.84 mmol) was reacted with NH₂OH·HCl (0.801 g, 11.53 mmol) and NaOAc (0.946 g, 11.53 mmol) in MeOH (10 mL) as per General Procedure 9 to provide 3-(4-(trifluoromethyl)phenyl)isoxazol-5(4H)-one in 100% yield (0.88 g).

Ethyl 3-(4-(trifluoromethyl)phenyl)isoxazol-5(4H)-one (0.88 g, 3.84 mmol) was reacted with POCl₃ (3.6 mL) and Et₃N (0.32 mL, 2.30 mmol) as per General Procedure 10 to provide 5-chloro-3-(4-(trifluoromethyl)phenyl)isoxazole in 96% yield (0.91 g).

5-Chloro-3-(4-(trifluoromethyl)phenyl)isoxazole (0.15 g, 0.61 mmol) was reacted with 5-aminonicotinonitrile (0.108 g, 0.91 mmol) and NaH (60%, 0.049 g, 1.22 mmol) in DMF (4 mL) as per General Procedure 2 Method 2 to provide 5-((3-(4-(trifluoromethyl)phenyl)isoxazol-5-yl)amino)picolinonitrile in 88% yield (0.176 g). ¹H NMR (401 MHz, DMSO-d₆) δ 11.06 (s, 1H), 8.62 (s, 1H), 8.14 (d, J=7.6 Hz, 2H), 8.00 (d, J=8.6 Hz, 1H), 7.90 (d, J=8.1 Hz, 2H), 7.83 (d, J=8.0 Hz, 1H), 6.76 (s, 1H). LCMS R_{f} (min)=3.858, MS m/z=329.1 [M−H]⁻.

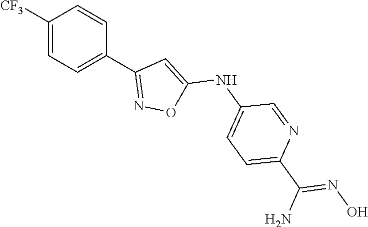 [SMILES: N/C(=N\O)c1ccc(Nc2cc(-c3ccc(C(F)(F)F)cc3)no2)cn1]

5-((3-(4-(Trifluoromethyl)phenyl)isoxazol-5-yl)amino)picolinonitrile (0.18 g, 0.545 mmol) was reacted with NH₂OH HCl (0.114 g, 1.64 mmol) and Et₃N (0.23 mL, 1.64 mmol) in MeOH (6 mL) for 2 h as per General Procedure 1 Method 2 to provide N′-hydroxy-5-((3-(4-(trifluoromethyl)phenyl)isoxazol-5-yl)amino)picolinimidamide in 67% yield (0.132 g). ¹H NMR (401 MHz, DMSO-d₆) δ 10.54 (s, 1H), 9.78 (s, 1H), 8.50 (s, 1H), 8.12 (d, J=7.3 Hz, 2H), 7.89-7.83 (m, 3H), 7.72 (d, J=7.5 Hz, 1H), 6.55 (s, 1H), 5.76 (br s, 2H). ¹³C NMR (101 MHz, DMSO-d₆) δ 165.5, 161.8, 149.3, 143.2, 136.7, 136.6, 133.1, 130.6, 130.3, 130.0, 129.7, 127.3, 125.9, 125.4, 123.7, 120.0, 80.4. LCMS R_{f} (min)=3.393, HRMS (ESI) calcd for C₁₆H₁₃F₃N₅O₂⁺ [M+H]⁺ 364.1021, found 364.1025.

### 90. 5-((5-(5-Chloropyridin-2-yl)oxazol-2-yl)amino)-N′-hydroxypicolinimidamide (Scheme 87)

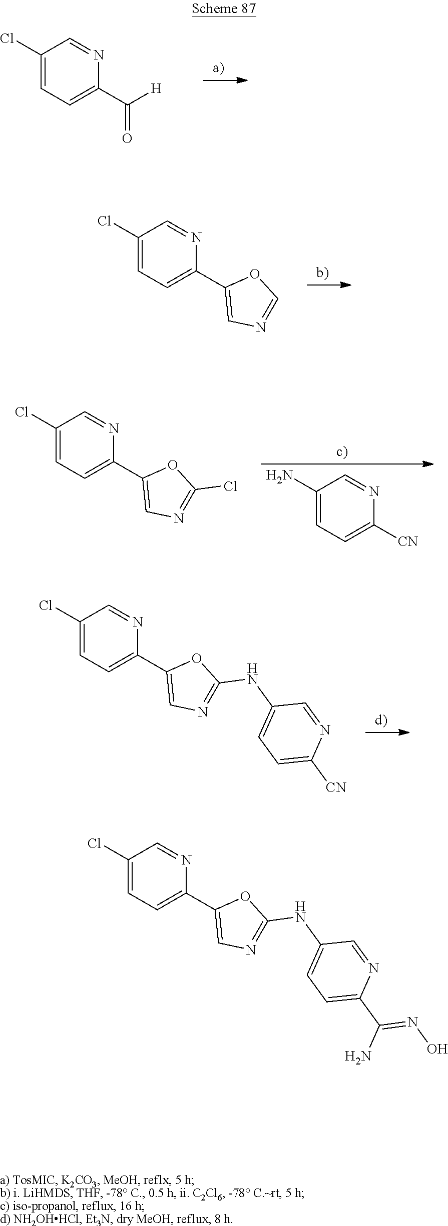

5-Chloropicolinaldehyde (3.09 g, 21.83 mmol) and TosMIC (5.11 g, 26.20 mmol) were reacted as per General Procedure 14, giving 5-(5-chloropyridin-2-yl)oxazole (4.0 g, 100%) as a white solid. ¹H NMR (401 MHz, DMSO-d₆) δ 8.69 (dd, J=2.5, 0.7 Hz, 1H), 8.57 (s, 1H), 8.07 (dd, J=8.5, 2.5 Hz, 1H), 7.84 (s, 1H), 7.81 (dd, J=8.5, 0.7 Hz, 1H); LCMS R_{f}(min)=2.55. MS m/z=181.1 [M+H]⁺.

5-(5-Chloropyridin-2-yl)oxazole (3.20 g, 17.7 mmol) was reacted with LiHMDS (21.3 mL, 21.3 mmol) then C₂Cl₆ (6.3 g, 26.6 mmol) in dry THF as per General Procedure 15 Method 1 to give 2-chloro-5-(5-chloropyridin-2-yl)oxazole (3.01 g, 79% yield) as a white solid. ¹H NMR (401 MHz, DMSO-d₆) δ 8.69 (dd, J=2.5, 0.6 Hz, 1H), 8.07 (dd, J=8.5, 2.5 Hz, 1H), 7.92 (s, 1H), 7.79 (dd, J=8.5, 0.5 Hz, 1H). LCMS R_{f}(min)=2.943, MS m/z=215.0 [M+H]⁺.

2-Chloro-5-(5-chloropyridin-2-yl)oxazole (0.5 g, 2.33 mmol) was reacted with 5-aminonicotinonitrile (0.416 g, 3.49 mmol) in iso-propanol (10 mL) as per General Procedure 2 Method 1 to provide 5-((5-(5-chloropyridin-2-yl)oxazol-2-yl)amino)picolinonitrile in 71% yield (0.492 g). ¹H NMR (401 MHz, DMSO-d₆) δ 8.83 (s, 1H), 8.62 (s, 1H), 8.32 (d, J=7.7 Hz, 1H), 7.00-7.98 (m, 2H), 7.76 (s, 1H), 7.66 (d, J=8.0 Hz, 1H). LCMS R_{f} (min)=2.794, MS m/z=298.1 [M+H]⁺.

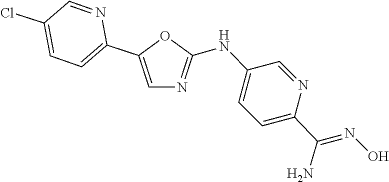 [SMILES: N/C(=N\O)c1ccc(Nc2ncc(-c3ccc(Cl)cn3)o2)cn1]

5-((5-(5-Chloropyridin-2-yl)oxazol-2-yl)amino)picolinonitrile (0.30 g, 1.01 mmol) was reacted with NH₂OH·HCl (0.561 g, 8.08 mmol) and Et₃N (1.13 mL, 8.08 mmol) in MeOH (10 mL) for 8 h as per General Procedure 1 Method 2 to provide 5-((5-(5-chloropyridin-2-yl)oxazol-2-yl)amino)-N′-hydroxypicolinimidamide in 92% yield (0.306 g). ¹H NMR (401 MHz, DMSO-d₆) δ 10.98 (s, 1H), 9.75 (s, 1H), 8.77 (s, 1H), 8.61 (s, 1H), 8.12-7.62 (m, 5H), 5.77 (br s, 2H). ¹³C NMR (101 MHz, DMSO-d₆) δ 156.8, 149.4, 148.2, 145.2, 143.5, 143.2, 137.0, 136.8, 136.1, 128.7, 126.9, 124.0, 119.7, 119.0. LCMS R_{f}(min)=2.241, HRMS (ESI) calcd for C₁₄H₁₂ClN₆O₂⁺ [M+H]⁺ 331.0705, found 331.0715.

### 91. rac-N-(2,3-dihydroxypropyl)-5-((5-(4-(trifluoromethyl)phenyl)oxazol-2-yl)amino)picolinimidamide (Scheme 88)

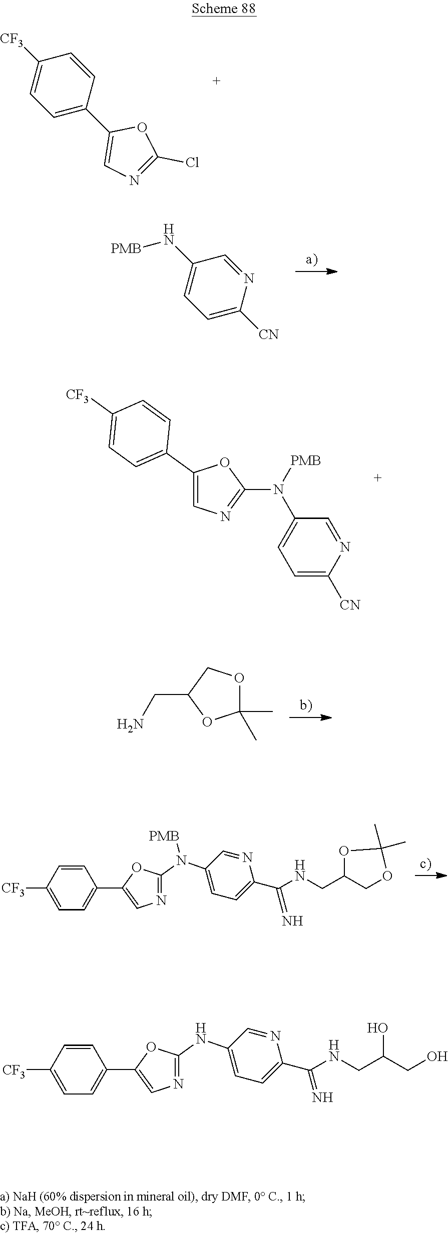

2-Chloro-5-(4-(trifluoromethyl)phenyl)oxazole (Intermediate I, 0.50 g, 2.02 mmol) was reacted with 5-((4-methoxybenzyl)amino)picolinonitrile (0.483 g, 2.02 mmol) and NaH (60%, 0.121 g, 3.03 mmol) in DMF (9 mL) as per General Procedure 2 Method 2 to provide 5-((4-methoxybenzyl)(5-(4-(trifluoromethyl)phenyl)oxazol-2-yl)amino)picolinonitrile in 98% yield (0.89 g). ¹H NMR (401 MHz, DMSO-d₆) δ 8.85 (d, J=2.3 Hz, 1H), 7.97 (dd, J=8.7, 2.8 Hz, 1H), 7.66-7.57 (m, 5H), 7.32 (s, 1H), 7.21 (d, J=8.7 Hz, 2H), 6.87 (d, J=8.7 Hz, 2H), 5.27 (s, 2H). 3.78 (s, 3H). LCMS R_{f}(min)=3.336, MS m/z=451.2 [M+H]⁺.

Sodium metal (0.044 g, 1.9 mmol) was dissolved in anhydrous MeOH (2 mL) and then added to a suspension of 5-((4-methoxybenzyl)(5-(4-(trifluoromethyl)phenyl)oxazol-2-yl)amino)picolinonitrile (0.172 g, 0.38 mmol) in anhydrous MeOH (2 mL). The reaction mixture was stirred at rt for 0.5 h then refluxed for a further 1 h. rac-(2,2-Dimethyl-1,3-dioxolan-4-yl)methanamine-HCl (0.255 g, 1.52 mmol) was added to the mixture. The reaction mixture was refluxed for 16 h to provide rac-N-((2,2-dimethyl-1,3-dioxolan-4-yl)methyl)-5-((4-methoxybenzyl)(5-(4-(trifluoromethyl)phenyl)oxazol-2-yl)amino)picolinimidamide in yield 84% (0.186 g). ¹H NMR (401 MHz, MeOH-d₄) δ 9.03 (s, 1H), 8.24 (d, J=7.3 Hz, 1H), 8.16 (d, J=8.2 Hz, 1H), 7.78 (d, J=8.0 Hz, 2H), 7.72 (d, J=7.8 Hz, 2H), 7.60 (s, 1H), 7.32 (d, J=8.0, 2H), 6.91 (d, J=8.2 Hz, 2H), 5.41 (s, 2H), 4.50 (m, 1H), 4.19 (m, 1H), 3.87-3.72 (m, 6H), 1.43 (s, 3H), 1.37 (s, 3H). LCMS R_{f}(min)=3.642, MS m/z=582.3 [M+H]⁺.

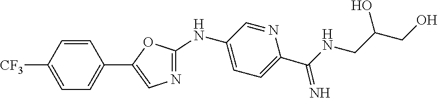 [SMILES: N=C(NCC(O)CO)c1ccc(Nc2ncc(-c3ccc(C(F)(F)F)cc3)o2)cn1]

rac-N-((2,2-Dimethyl-1,3-dioxolan-4-yl)methyl)-5-((4-methoxybenzyl)(5-(4-(trifluoromethyl)phenyl)oxazol-2-yl)amino)picolinimidamide (0.05 g, 0.086 mmol) was reacted with TFA (5 mL) at 70° C. for 24 h as per General Procedure 11, except without addition of Et₃SiH or anisole, to provide rac-N-(2,3-dihydroxypropyl)-5-((5-(4-(trifluoromethyl)phenyl)oxazol-2-yl)amino)picolinimidamide in 72% yield (0.026 g). ¹H NMR (401 MHz, MeOH-d₄) δ 8.94 (dd, J=2.6, 0.5 Hz, 1H), 8.51 (dd, J=8.8, 2.6 Hz, 1H), 8.17 (dd, J=8.8, 0.5 Hz, 1H), 7.84 (d, J=8.2 Hz, 2H), 7.74 (d, J=8.4 Hz, 2H), 7.59 (s, 1H), 4.00 (m, 1H), 3.73-3.61 (m, 4H). ¹³C NMR (101 MHz, MeOH-d₄) δ 146.1, 141.6, 140.2, 137.1, 132.8, 127.1, 126.9, 125.3, 124.5, 124.40, 124.36, 124.3, 71.1, 68.1, 64.8. LCMS R_{f}(min)=3.128, HRMS (ESI) calcd for C₁₉H₁₉F₃N₅O₃⁺ [M+H]⁺ 422.1440, found 422.1449.

### 92. 5-((5-(4-(Trifluoromethyl)phenyl)oxazol-2-yl)amino)picolinohydrazide (Scheme 89)

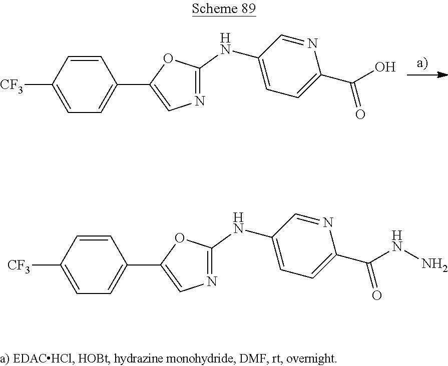 [SMILES: O=C(O)c1ccc(Nc2ncc(-c3ccc(C(F)(F)F)cc3)o2)cn1 ; NNC(=O)c1ccc(Nc2ncc(-c3ccc(C(F)(F)F)cc3)o2)cn1]

EDAC·HCl (0.071 g, 0.371 mmol) and HOBt (0.054 g, 0.4 mmol) were added to a suspension of 5-((5-(4-(trifluoromethyl)phenyl)oxazol-2-yl)amino)picolinic acid (Intermediate B, 0.10 g, 0.286 mmol) in DMF (5.0 mL). The resulting mixture was stirred at rt for 3 h. Hydrazine monohydrate (0.022 g, 0.43 mmol) was then added to the reaction mixture and stirring was continued overnight. The reaction mixture was concentrated under reduced pressure to obtain a gummy solid, which was purified using preparative HPLC in a 95% A: 5% B to 100% B solvent system. The TFA and acetonitrile were removed through rotary evaporation and H₂O through freeze-drying, providing 5-((5-(4-(trifluoromethyl)phenyl)oxazol-2-yl)amino)picolinohydrazide as a beige solid (0.066 g, 63% yield). ¹H NMR (400 MHz, DMSO-d₆) δ 11.23 (m, 1H), 8.83 (m, 1H), 8.34 (m, 1H), 8.09 (m, 1H), 7.82-7.79 (m, 5H). ¹³C NMR (101 MHz, DMSO-d₆) δ 163.3, 159.0, 156.1, 143.5, 139.0, 138.5, 137.6, 137.3, 131.4, 126.1, 125.6, 125.3, 123.7, 123.5, 123.2. LCMS R_{f}(min)=3.931. HRMS (ESI) calcd for C₁₆H₁₃F₃N₅O₂⁺ [M+H]⁺ 364.1021, found 364.1021.

### 93. N′-formyl-5-((5-(4-(trifluoromethyl)phenyl)oxazol-2-yl)amino)picolinohydrazide (Scheme 90)

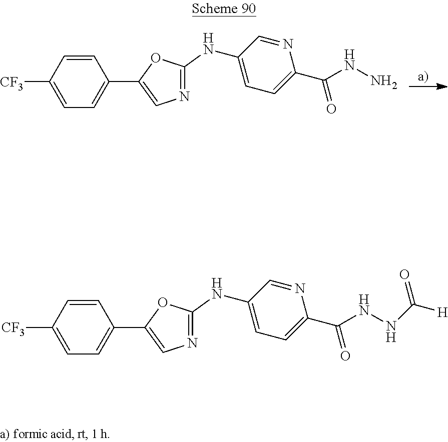 [SMILES: NNC(=O)c1ccc(Nc2ncc(-c3ccc(C(F)(F)F)cc3)o2)cn1 ; O=CNNC(=O)c1ccc(Nc2ncc(-c3ccc(C(F)(F)F)cc3)o2)cn1]

5-((5-(4-(Trifluoromethyl)phenyl)oxazol-2-yl)amino)picolinohydrazide (0.05 g, 13.7 mmol) was added to formic acid (5 mL) at rt. The mixture was stirred for 1 h. The volatiles were removed under reduced pressure to give a residue that was purified using preparative HPLC in a 95% A: 5% B to 100% B solvent system. The TFA and acetonitrile were removed through rotary evaporation and H₂O through freeze-drying, providing 5-((5-(4-(trifluoromethyl)phenyl)oxazol-2-yl)amino)picolinohydrazide as a beige solid (0.032 g, 60% yield). ¹H NMR (401 MHz, DMSO-d₆) δ 11.18 (s, 1H), 10.33 (s, 1H), 10.07 (s, 1H), 8.83 (J=2.3, 1H), 8.32 (d, J=8.7 Hz, 1H), 8.09 (s, 1H), 8.04 (d, J=8.7 Hz, 1H), 7.82 (br s, 4H), 7.79 (s, 1H). ¹³C NMR (101 MHz, DMSO-d₆) δ 162.5, 159.7, 156.2, 141.7, 138.6, 137.4, 131.5, 126.2, 125.3, 123.5, 123.4, 123.2. LCMS R_{f}(min)=3.492, HRMS (ESI) calcd for C₁₇H₁₃F₃N₅O₃⁺ [M+H]⁺ 392.0970, found 392.0974.

### 94. N′-Methyl-5-((5-(4-(trifluoromethyl)phenyl)oxazol-2-yl)amino)picolinohydrazide (Scheme 91)

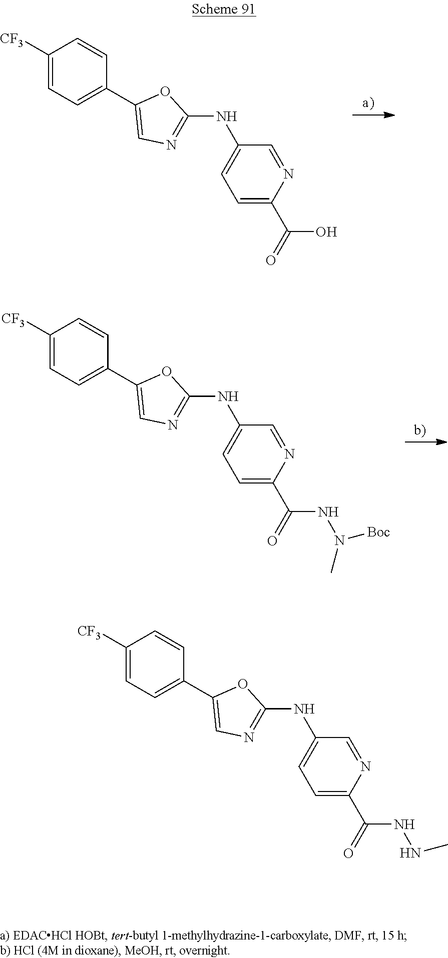 [SMILES: O=C(O)c1ccc(Nc2ncc(-c3ccc(C(F)(F)F)cc3)o2)cn1 ; CN(NC(=O)c1ccc(Nc2ncc(-c3ccc(C(F)(F)F)cc3)o2)cn1)C(=O)OC(C)(C)C ; CNNC(=O)c1ccc(Nc2ncc(-c3ccc(C(F)(F)F)cc3)o2)cn1]

EDAC·HCl (0.071 g, 0.371 mmol) and HOBt (0.054 g, 0.4 mmol) were added to a suspension of 5-((5-(4-(trifluoromethyl)phenyl)oxazol-2-yl)amino)picolinic acid (Intermediate B, 0.10 g, 0.286 mmol) in DMF (5.0 mL). The resulting mixture was stirred at rt for 3 h. tert-Butyl 1-methylhydrazine-1-carboxylate (0.084 g, 0.572 mmol) was then added to the reaction mixture and stirring was continued overnight. The reaction mixture was concentrated under reduced pressure to obtain a gummy solid, which was purified using flash chromatography to give tert-butyl 1-methyl-2-(5-((5-(4-(trifluoromethyl)phenyl)oxazol-2-yl)amino)picolinoyl)hydrazine-1-carboxylate as a white solid (0.117 g, 86% yield). ¹H NMR (400 MHz, DMSO-d₆) δ 7.99 (s, 1H), 7.97 (s, 1H), 7.73 (s, 1H), 7.71 (s, 1H), 7.56-7.52 (m, 2H), 7.43-7.39 (m, 2H), 2.54 (s, 9H). LCMS R_{f}(min)=3.116, MS m/z=476.1 [M−H]⁻.

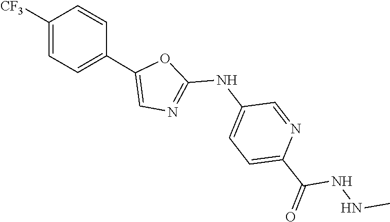 [SMILES: CNNC(=O)c1ccc(Nc2ncc(-c3ccc(C(F)(F)F)cc3)o2)cn1]

HCl (0.5 mL, 2.0 mmol, 4 M in dioxane) was added to a solution of tert-butyl 1-methyl-2-(5-((5-(4-(trifluoromethyl)phenyl)oxazol-2-yl)amino)picolinoyl)hydrazine-1-carboxylate (0.06, 0.126 mmol) in MeOH (5 mL) at rt. The mixture was stirred overnight. The volatiles were removed under reduced pressure to give a residue that was purified using preparative HPLC in a 95% A: 5% B to 100% B solvent system. The TFA and acetonitrile were removed through rotary evaporation and H₂O through freeze-drying, providing N′-methyl-5-((5-(4-(trifluoromethyl)phenyl)oxazol-2-yl)amino)picolinohydrazide (0.038 g, 80% yield). ¹H NMR (401 MHz, DMSO-d₆) δ 11.27 (s, 1H), 8.86 (d, J=2.4, 1H), 8.36 (dd, J=8.7, 2.5 Hz, 1H), 8.10 (d, J=8.7 Hz, 1H), 7.83 (br s, 4H), 7.81 (s, 1H), 2.80 (s, 3H). ¹³C NMR (101 MHz, DMSO-d₆) δ 162.7, 156.1, 143.5, 140.4, 139.1, 137.6, 131.4, 126.1, 125.3, 123.8, 123.5, 123.2, 122.9, 66.4. LCMS R_{f} (min)=3.893, HRMS (ESI) calcd for C₁₇H₁₅F₃N₅O₂⁺ [M+H]⁺ 378.1178, found 378.1190.

### 95. (R)—N-(2,3-dihydroxypropyl)-5-((5-(5-(trifluoromethyl)pyridin-2-yl)oxazol-2-yl)amino)picolinamide (Scheme 92)

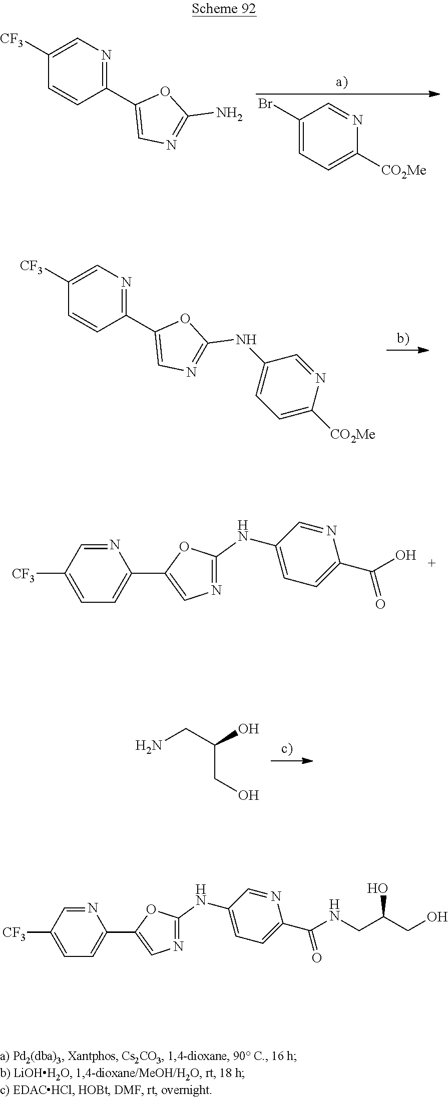 [SMILES: Nc1ncc(-c2ccc(C(F)(F)F)cn2)o1 ; COC(=O)c1ccc(Br)cn1 ; COC(=O)c1ccc(Nc2ncc(-c3ccc(C(F)(F)F)cn3)o2)cn1 ; O=C(O)c1ccc(Nc2ncc(-c3ccc(C(F)(F)F)cn3)o2)cn1 ; NC[C@@H](O)CO ; O=C(NC[C@@H](O)CO)c1ccc(Nc2ncc(-c3ccc(C(F)(F)F)cn3)o2)cn1]

5-(5-(trifluoromethyl)pyridin-2-yl)oxazol-2-amine (0.6 g, 2.62 mmol) and methyl 5-bromopicolinate (0.68 g, 3.14 mmol) were reacted as per General Procedure 4 Method 1, using 5 mol % Pd₂(dba)₃, 10 mol % Xantphos, 1.5 eq Cs₂CO₃ in 1,4-dioxane at 90° C. for 16 h to give methyl 5-((5-(5-(trifluoromethyl)pyridin-2-yl)oxazol-2-yl)amino)picolinate (0.63 g, 63%). ¹H NMR (401 MHz, DMSO-d₆) δ 11.45 (br s, 1H), 8.94 (s, 1H), 8.84 (d, J=2.4 Hz, 1H), 8.32 (dd, J=8.7, 2.6 Hz, 1H), 8.27 (dd, J=8.4, 1.8 Hz, 1H), 8.09 (d, J=8.7 Hz, 1H), 7.94 (s, 1H), 7.81 (d, J=8.4 Hz, 1H), 3.85 (s, 3H). LCMS R_{f}(min)=2.748, MS m/z=365.1 [M+H]⁺.

LiOH·H₂O (0.07 g, 1.65 mmol) was added to a suspension of methyl 5-((5-(5-(trifluoromethyl)pyridin-2-yl)oxazol-2-yl)amino)picolinate (0.20 g, 0.55 mmol) in 1,4-dioxane (2 mL), MeOH (2 mL) and H₂O (2 mL). The reaction mixture was stirred at rt for 18 h. The volatiles were removed in vacuo and brine was added (2 mL). This residue was then acidified with 6 M HCl aq. (2 mL) at 0° C. then filtered, washed with H₂O (4 mL) and Et₂O (4 mL) to give 5-((5-(5-(trifluoromethyl)pyridin-2-yl)oxazol-2-yl)amino)picolinic acid (0.18 g, 94% yield). ¹H NMR (401 MHz, DMSO-d₆) δ 11.42 (s, 1H), 8.94 (s, 1H), 8.84 (d, J=2.4 Hz, 1H), 8.33-8.23 (m, 2H), 8.08 (d, J=8.7 Hz, 1H), 7.93 (s, 1H), 7.81 (d, J=8.4 Hz, 1H). LCMS R_{f}(min)=2.511, MS m/z=351.0 [M+H]⁺.

EDAC·HCl (0.089 g, 0.46 mmol) and HOBt (0.068 g, 0.50 mmol) were added to a suspension of 5-((5-(5-(trifluoromethyl)pyridin-2-yl)oxazol-2-yl)amino)picolinic acid (0.125 g, 0.36 mmol) in DMF (8.0 mL). The resulting mixture was stirred at rt for 3 h. (R)-3-Aminopropane-1,2-diol-HCl (0.066 g, 0.72 mmol) was then added to the reaction mixture and stirring was continued overnight. The reaction mixture was concentrated under reduced pressure to obtain a gummy solid, which was purified using preparative HPLC in a 95% A: 5% B to 100% B solvent system. The TFA and acetonitrile were removed through rotary evaporation and H₂O through freeze-drying, providing (R)—N-(2,3-dihydroxypropyl)-5-((5-(5-(trifluoromethyl)pyridin-2-yl)oxazol-2-yl)amino)picolinamide as a beige solid (0.07 g, 46% yield). ¹H NMR (400 MHz, DMSO-d₆) δ 11.32 (br s, 1H), 8.93 (m, 1H), 8.80 (d, J=2.2 Hz, 1H), 8.46 (t, J=5.9 Hz, 1H), 8.30 (dd, J=8.6, 2.0 Hz, 1H), 8.26 (dd, J=8.5, 1.9 Hz, 1H), 8.04 (d, J=8.6, 1H), 7.92 (s, 1H), 7.80 (d, J=8.4 Hz, 1H), 4.95 (d, J=4.9 Hz, 1H), 4.67 (t, J=5.6 Hz, 1H), 3.61 (m, 1H), 3.52-3.46 (m, 2H), 3.29-3.19 (m, 2H). ¹³C NMR (101 MHz, DMSO-d₆) δ 163.7, 157.2, 149.9, 146.6, 143.6, 143.0, 138.0, 137.4, 134.8, 129.1, 125.2, 123.9, 122.6, 122.5, 119.78, 117.8, 70.2, 64.0, 42.4. LCMS R_{f}(min)=3.218. HRMS (ESI) calcd for C₁₈H₁₇F₃N₅O₄⁺ [M+H]⁺ 424.1233, found 424.1232.

### 96. (R)—N-(2,3-dihydroxypropyl)-5-((4-methyl-5-(4-(trifluoromethyl)phenyl)oxazol-2-yl)amino)picolinamide (Scheme 93)

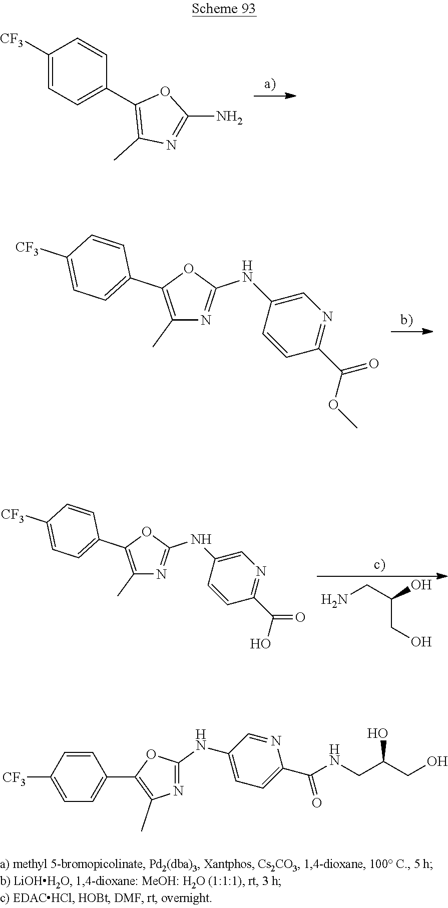 [SMILES: Cc1nc(N)oc1-c1ccc(C(F)(F)F)cc1 ; COC(=O)c1ccc(Nc2nc(C)c(-c3ccc(C(F)(F)F)cc3)o2)cn1 ; Cc1nc(Nc2ccc(C(=O)O)nc2)oc1-c1ccc(C(F)(F)F)cc1 ; NC[C@@H](O)CO ; Cc1nc(Nc2ccc(C(=O)NC[C@@H](O)CO)nc2)oc1-c1ccc(C(F)(F)F)cc1]

4-Methyl-5-(4-(trifluoromethyl)phenyl)oxazol-2-amine (0.23 g, 0.95 mmol) was reacted with methyl 5-bromopicolinate (0.246 g, 1.14 mmol), Pd₂(dba)₃ (0.043 g, 0.048 mmol), Xantphos (0.055 g, 0.095 mmol) and Cs₂CO₃ (0.464 g, 1.43 mmol) in 1,4-dioxane (8 mL) at 100° C. for 5 h as per General Procedure 4 Method 1 to provide methyl 5-((4-methyl-5-(4-(trifluoromethyl)phenyl)oxazol-2-yl)amino)picolinate in 89% yield (0.32 g). ¹H NMR (401 MHz, DMSO-d₆) δ 8.07 (d, J=8.4 Hz, 2H), 7.85 (d, J=8.3 Hz, 2H), 7.85 (m, 1H), 7.40 (m, 1H), 7.10 (s, 1H), 3.84 (s, 3H), 2.40 (s, 3H). LCMS R_{f}(min)=3.951, MS m/z=377.9 [M+H]⁺. LiOH·H₂O (0.40 g, 1.60 mmol) in H₂O (1 mL) was added to a solution of methyl 5-((4-methyl-5-(4-(trifluoromethyl)phenyl)oxazol-2-yl)amino)picolinate (0.15 g, 0.40 mmol) in 1,4-dioxane (1 mL) and MeOH (1 mL). The reaction mixture was stirred at rt for 3 h. Volatile solvents were removed in vacuo and to the obtained suspension was added brine (2 mL), followed by HCl (2 mL, 6 M) dropwise at 0° C. The resulting precipitate was filtered and washed with H₂O (2×1 mL), providing 5-((4-methyl-5-(4-(trifluoromethyl)phenyl)oxazol-2-yl)amino)picolinic acid in 84% yield (0.122 g). ¹H NMR (400 MHz, DMSO-d₆) δ 8.05 (d, J=8.2 Hz, 2H), 7.94 (d, J=8.3 Hz, 2H), 7.83 (d, J=3.0 Hz, 1H), 7.40 (dd, J=9.0, 3.1 Hz, 1H), 7.10 (d, J=8.9 Hz, 1H), 3.72 (s, 3H). LCMS R_{f}(min)=3.379, MS m/z=364.0 [M+H]⁺.

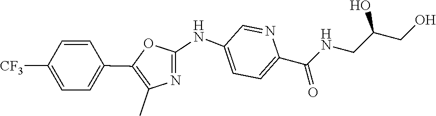 [SMILES: Cc1nc(Nc2ccc(C(=O)NC[C@@H](O)CO)nc2)oc1-c1ccc(C(F)(F)F)cc1]

EDAC·HCl (0.027 g, 0.14 mmol) and HOBt (0.021 g, 0.154 mmol) were added to a suspension of 5-((4-methyl-5-(4-(trifluoromethyl)phenyl)oxazol-2-yl)amino)picolinic acid (0.04 g, 0.11 mmol) in DMF (2 mL). The resulting mixture was stirred at rt for 3 h. (R)-3-Aminopropane-1,2-diol-HCl (0.02 g, 0.22 mmol) was then added to the reaction mixture and stirring was continued overnight. The reaction mixture was concentrated under reduced pressure to obtain a gummy solid, which was purified using preparative HPLC in a 95% A: 5% B to 100% B solvent system. The TFA and acetonitrile were removed through rotary evaporation and H₂O through freeze-drying, providing (R)—N-(2,3-dihydroxypropyl)-5-((4-methyl-5-(4-(trifluoromethyl)phenyl)oxazol-2-yl)amino)picolinamide as a beige solid (0.032 g, 67% yield). ¹H NMR (400 MHz, DMSO-d₆) δ 11.02 (br s, 1H), 8.79 (d, J=2.4 Hz, 1H), 8.44 (t, J=5.8 Hz, 1H), 8.29 (dd, J=8.6, 2.6 Hz, 1H), 8.04 (d, J=8.6 Hz, 1H), 7.82 (d, J=8.5 Hz, 2H), 7.73 (d, J=8.3 Hz, 2H), 4.94 (d, J=4.9 Hz, 1H), 4.66 (t, J=5.5 Hz, 1H), 3.62 (m, 1H), 3.52-3.46 (m, 2H), 3.25-3.19 (m, 2H). ¹³C NMR (101 MHz, DMSO-d₆) δ 163.7, 154.7, 142.7, 138.2, 137.5, 137.2, 134.6, 132.4, 126.0, 124.2, 123.5, 122.6, 70.2, 63.9, 42.4, 13.7. LCMS R_{f}(min)=2.740. HRMS (ESI) calcd for C₂₀H₂₀F₃N₄O₄⁺ [M+H]⁺ 437.1437, found 437.1442.

### 97. (R)-5-((5-(3,4-Difluorophenyl)oxazol-2-yl)amino)-N-(2,3-dihydroxypropyl)picolinamide (Scheme 94)

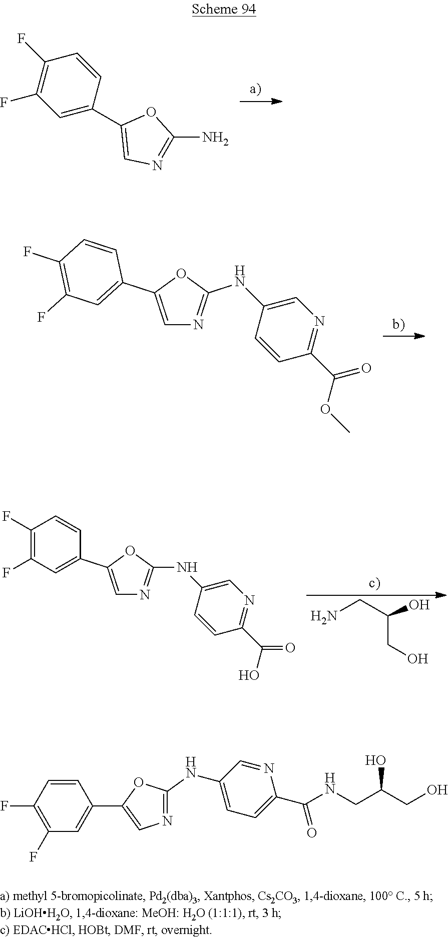 [SMILES: Nc1ncc(-c2ccc(F)c(F)c2)o1 ; COC(=O)c1ccc(Nc2ncc(-c3ccc(F)c(F)c3)o2)cn1 ; O=C(O)c1ccc(Nc2ncc(-c3ccc(F)c(F)c3)o2)cn1 ; NC[C@@H](O)CO ; O=C(NC[C@@H](O)CO)c1ccc(Nc2ncc(-c3ccc(F)c(F)c3)o2)cn1]

5-(3,4-Difluorophenyl)oxazol-2-amine (0.25 g, 1.27 mmol) was reacted with methyl 5-bromopicolinate (0.33 g, 1.53 mmol), Pd₂(dba)₃ (0.058 g, 0.064 mmol), Xantphos (0.044 g, 0.076 mmol) and Cs₂CO₃ (0.622 g, 1.91 mmol) in 1,4-dioxane (10 mL) at 100° C. for 5 h as per General Procedure 4 Method 1 to provide methyl 5-((5-(3,4-difluorophenyl)oxazol-2-yl)amino)picolinate in 88% yield (0.37 g). ¹H NMR (401 MHz, DMSO-d₆) δ 8.54 (s, 1H), 8.21 (dd, J=8.8, 2.5 Hz, 1H), 7.92 (d, J=8.8 Hz, 1H), 7.79 (m, 1H), 7.60 (m, 1H), 7.51 (s, 1H), 7.46 (s, 1H), 7.36 (m, 1H), 3.80 (s, 3H). LCMS R_{f} (min)=3.068, MS m/z=332.0 [M+H]⁺.

LiOH·H₂O (0.101 g, 4.23 mmol) in H₂O (2 mL) was added to a solution of methyl 5-((5-(3,4-difluorophenyl)oxazol-2-yl)amino)picolinate (0.35 g, 1.05 mmol) in 1,4-dioxane (2 mL) and MeOH (2 mL). The reaction mixture was stirred at rt for 3 h. Organic solvents were removed in vacuo and to the obtained suspension was added brine (2 mL), followed by HCl (2 mL, 6 M) dropwise at 0° C. The resulting precipitate was filtered and washed with H₂O (2×2 mL), providing 5-((5-(3,4-difluorophenyl)oxazol-2-yl)amino)picolinic acid in 80% yield (0.267 g). ¹H NMR (401 MHz, DMSO-d₆) δ 11.30 (br s, 1H), 8.87 (d, J=2.4 Hz, 1H), 8.33 (dd, J=8.7, 2.5 Hz, 1H), 8.10 (d, J=8.7 Hz, 1H), 7.70 (m, 1H), 7.64 (s, 1H), 7.54 (m, 1H), 7.45 (m, 1H). LCMS R_{f}(min)=3.003, MS m/z=318.0 [M+H]⁺.

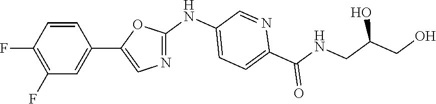 [SMILES: O=C(NC[C@@H](O)CO)c1ccc(Nc2ncc(-c3ccc(F)c(F)c3)o2)cn1]

5-((5-(3,4-Difluorophenyl)oxazol-2-yl)amino)picolinic acid (0.10 g, 0.315) was reacted with EDAC·HCl (0.079 g, 0.41 mmol), (0.059 g, 0.441 mmol) and (R)-3-aminopropane-1,2-diol (0.034 g, 0.378 mmol) as per step c, Scheme 92 to provide (R)-5-((5-(3,4-difluorophenyl)oxazol-2-yl)amino)-N-(2,3-dihydroxypropyl)picolinamide in 35% yield (0.043 g). ¹H NMR (400 MHz, DMSO-d₆) δ 11.04 (s, 1H), 8.78 (d, J=2.3 Hz, 1H), 8.45 (t, J=5.9 Hz, 1H), 8.29 (dd, J=8.6, 2.6 Hz, 1H), 8.03 (d, J=8.7 Hz, 1H), 7.69 (ddd, J=11.7, 7.6, 2.1 Hz, 1H), 7.62 (s, 1H), 7.55 (dt, J=10.5, 8.5 Hz, 1H), 7.43 (m, 1H), 4.95 (d, J=5.0 Hz, 1H), 4.66 (t, J=5.8 Hz, 1H), 3.61 (dt, J=10.8, 5.3 Hz, 1H). *Resonances that overlapped with H₂O residue were not assigned. ¹³C NMR (101 MHz, DMSO-d₆) δ 163.9, 155.8, 142.6, 138.2, 137.1, 123.8, 123.4, 122.6, 119.6, 118.6, 112.1, 111.9, 70.2, 63.9, 42.3. LCMS R_{f}(min)=2.949. HRMS (ESI) calcd for C₁₈H₁₇F₂N₄O₄⁺ [M+H]⁺ 391.1218, found 391.1222.

### 98. (R)—N-(2,3-dihydroxypropyl)-5-((5-(4-fluorophenyl)oxazol-2-yl)amino)picolinamide (Scheme 95)

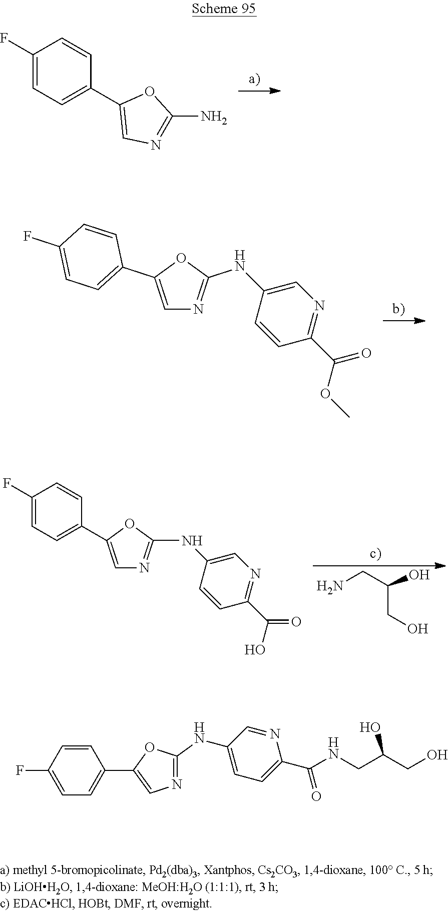 [SMILES: Nc1ncc(-c2ccc(F)cc2)o1 ; COC(=O)c1ccc(Nc2ncc(-c3ccc(F)cc3)o2)cn1 ; O=C(O)c1ccc(Nc2ncc(-c3ccc(F)cc3)o2)cn1 ; NC[C@@H](O)CO ; O=C(NC[C@@H](O)CO)c1ccc(Nc2ncc(-c3ccc(F)cc3)o2)cn1]

5-(4-Fluorophenyl)oxazol-2-amine (0.17 g, 0.95 mmol) was reacted with methyl 5-bromopicolinate (0.247 g, 1.15 mmol), Pd₂(dba)₃ (0.043 g, 0.048 mmol), Xantphos (0.033 g, 0.057 mmol) and Cs₂CO₃ (0.464 g, 1.43 mmol) in 1,4-dioxane (10 mL) at 100° C. for 5 h as per General Procedure 4 Method 1 to provide methyl 5-((5-(4-fluorophenyl)oxazol-2-yl)amino)picolinate in 82% yield (0.243 g). ¹H NMR (401 MHz, DMSO-d₆) δ 8.56 (s, 1H), 8.22 (m, 1H), 7.92 (d, J=8.7 Hz, 1H), 7.58 (m, 2H), 7.42 (s, 1H), 7.25 (m, 2H), 3.80 (s, 3H). LCMS R_{f}(min)=2.767, MS m/z=314.1 [M+H]⁺.

LiOH·H₂O (0.058 g, 2.43 mmol) in H₂O (3 mL) was added to a solution of methyl 5-((5-(4-fluorophenyl)oxazol-2-yl)amino)picolinate (0.19 g, 0.61 mmol) in 1,4-dioxane (3 mL) and MeOH (3 mL). The reaction mixture was stirred at rt for 3 h. Volatile solvents were removed in vacuo and to the obtained suspension was added brine (2 mL), followed by HCl (2 mL, 6 M) dropwise at 0° C. The resulting precipitate was filtered and washed with H₂O (2×2 mL), providing 5-((5-(4-fluorophenyl)oxazol-2-yl)amino)picolinic acid in 86% yield (0.158 g). ¹H NMR (401 MHz, DMSO-d₆) δ 11.08 (s, 1H), 8.82 (d, J=2.3 Hz, 1H), 8.29 (dd, J=8.7, 2.6 Hz, 1H), 8.06 (d, J=8.6 Hz, 1H), 7.80-7.60 (m, 2H), 7.54 (s, 1H), 7.31 (dd, J=12.4, 5.5 Hz, 2H). LCMS R_{f} (min)=2.966, MS m/z=300.1 [M+H]⁺.

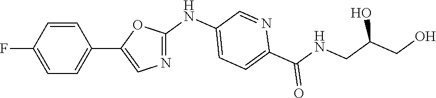 [SMILES: O=C(NC[C@@H](O)CO)c1ccc(Nc2ncc(-c3ccc(F)cc3)o2)cn1]

5-((5-(4-Fluorophenyl)oxazol-2-yl)amino)picolinic acid (0.10 g, 0.334) was reacted with EDAC·HCl (0.083 g, 0.434 mmol), HOBt (0.063 g, 0.468 mmol) and (R)-3-aminopropane-1,2-diol (0.036 g, 0.40 mmol) as per step c, Scheme 92 to provide (R)—N-(2,3-dihydroxypropyl)-5-((5-(4-fluorophenyl)oxazol-2-yl)amino)picolinamide in 44% yield (0.055 g). ¹H NMR (400 MHz, DMSO-d₆) δ 10.97 (br s, 1H), 8.78 (d, J=2.6 Hz, 1H), 8.44 (t, J=5.9 Hz, 1H), 8.29 (dd, J=8.7, 2.6 Hz, 1H), 8.03 (d, J=8.6 Hz, 1H), 7.66 (dd, J=8.8, 5.3 Hz, 2H), 7.53 (s, 1H), 7.31 (t, J=8.9 Hz, 2H), 4.95 (d, J=5.0 Hz, 1H), 4.66 (t, J=5.7 Hz, 1H), 3.61 (m, 1H). *Resonance overlapping with H₂O residue were not assigned. ¹³C NMR (101 MHz, DMSO-d₆) δ 160.8, 152.1, 144.3, 142.6, 139.3, 138.3, 137.1, 130.4, 129.6, 123.2, 122.2, 120.6, 118.5, 117.6, 115.3, 111.7, 110.2, 70.66, 65.1, 42.70. LCMS R_{f}(min)=2.965. HRMS (ESI) calcd for C₁₈H₁₈FN₄O₄⁺ [M+H]⁺ 373.1312, found 373.1306.

### 99. N′-Hydroxy-5-((5-(5-(trifluoromethoxy)pyridin-2-yl)oxazol-2-yl)amino)picolinimidamide (Scheme 96)

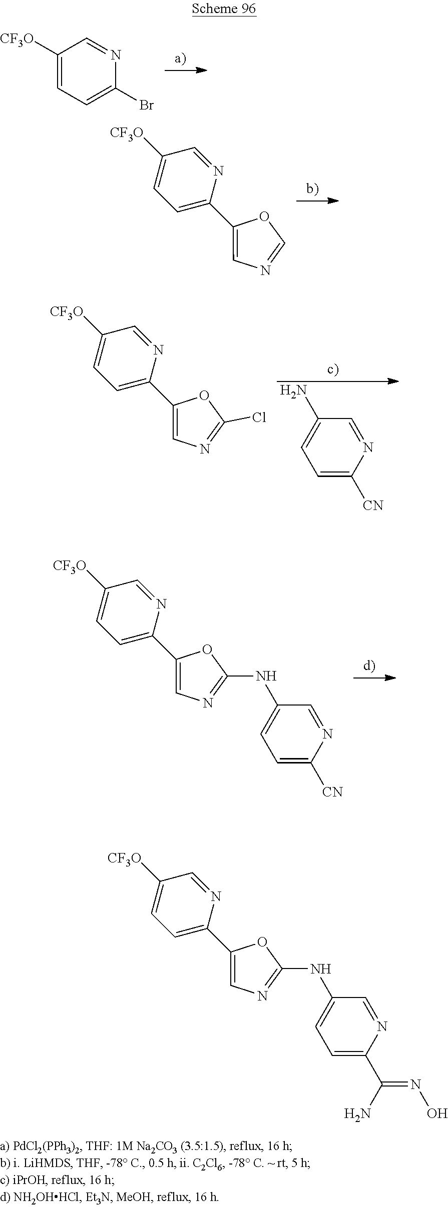

To a degassed biphasic solution of THF (3.5 mL) and 1 M Na₂CO₃ (aq.) (1.5 mL), was added 2-bromo-5-(trifluoromethoxy)pyridine (1.20 g, 4.96 mmol), 5-(4,4,5,5-tetramethyl-1,3,2-dioxaborolan-2-yl)oxazole (1.0 M in THF, 5.45 mL, 5.45 mmol) and PdCl₂(PPh₃)₂ (348 mg, 0.496 mmol). The mixture was reacted according to General Procedure 12 Method 1 to afford 5-(5-(trifluoromethoxy)pyridin-2-yl)oxazole as a brown solid (57.8 mg, 5.1%). ¹H NMR (401 MHz, CDCl₃) δ 8.52 (d, J=2.6 Hz, 1H), 7.95 (s, 1H), 7.72-7.66 (m, 2H), 7.63-7.56 (m, 1H).

5-(5-(Trifluoromethoxy)pyridin-2-yl)oxazole (0.33 g, 1.43 mmol) was reacted with LiHMDS (1.57 mL, 1.57 mmol, 1 M in hexane) and C₂Cl₆ (0.51 g, 2.15 mmol) in THF (6 mL) as per General Procedure 15 Method 1 to provide 2-chloro-5-(5-(trifluoromethoxy)pyridin-2-yl)oxazole (0.322 g, 85%) as a white solid. ¹H NMR (401 MHz, CDCl₃) δ 8.51 (s, 1H), 7.76-7.49 (m, 3H), LCMS R_{f}(min)=3.065, MS m/z=265.0 [M+H]⁺.

2-Chloro-5-(5-(trifluoromethoxy)pyridin-2-yl)oxazole (185 mg, 0.699 mmol, 1.0 eq.) and 5-aminonicotinonitrile (125 mg, 1.05 mmol, 1.5 eq.) in 2-propanol (5 mL) was reacted according to General Procedure 2 Method 1 to afford 5-((5-(5-(trifluoromethoxy)pyridin-2-yl)oxazol-2-yl)amino)picolinonitrile as a beige solid (205.1 mg, 85%) ¹H NMR (401 MHz, DMSO-d₆) δ 8.85 (d, J=2.6 Hz, 1H), 8.67 (d, J=2.8 Hz, 1H), 8.33 (dd, J=8.7, 2.6 Hz, 1H), 8.00 (m, 2H), 7.83-7.71 (m, 2H). LCMS R_{f}(min)=2.905, MS m/z=348.1 [M+H]⁺.

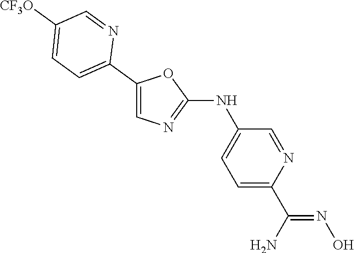

5-((5-(5-(Trifluoromethoxy)pyridin-2-yl)oxazol-2-yl)amino)picolinonitrile (285 mg, 0.821 mmol, 1.0 eq.), NH₂OH·HCl (456 mg, 6.57 mmol, 8.0 eq.) and Et₃N (0.916 mL, 6.57 mmol 8.0 eq.) in anhydrous MeOH (10 mL) was reacted according to General Procedure 1 Method 2 to afford N′-hydroxy-5-((5-(5-(trifluoromethoxy)pyridin-2-yl)oxazol-2-yl)amino)picolinimidamide as a beige solid (0.027 g, 22%). ¹H NMR (401 MHz, DMSO-d₆) δ 9.75 (s, 1H), 8.79 (d, J=2.4 Hz, 1H), 8.66 (d, J=2.6 Hz, 1H), 8.13 (dd, J=8.8, 2.6 Hz, 1H), 8.02-7.94 (m, 1H), 7.84 (d, J=8.8 Hz, 1H), 7.74 (m, 2H), 5.76 (s, 2H); ¹³C NMR (101 MHz, DMSO) δ 157.0, 149.4, 145.9, 143.5, 143.4, 143.2, 143.0, 136.9, 136.1, 130.4, 130.4, 127.2, 124.0, 119.7, 119.0. LCMS R_{f}(min)=2.385, MS m/z=381.1 [M+H]⁺. HRMS (ESI) calcd for C₁₅H₁₂F₃N₆O₃⁺ ([M+H]⁺ 381.0917, found 381.0935.

### 100. 5-((5-(4-Chlorophenyl)oxazol-2-yl)amino)-N′-hydroxypicolinimidamide (Scheme 97)

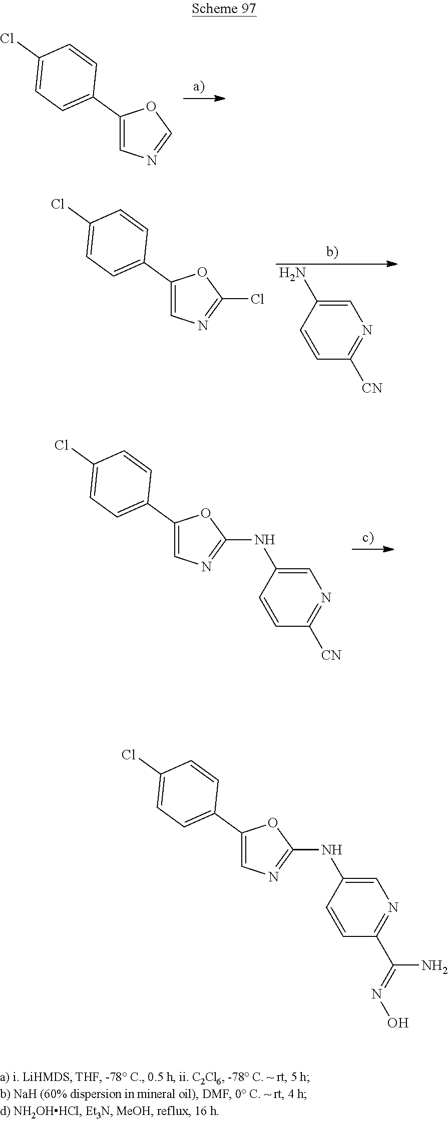 [SMILES: Clc1ccc(-c2cnco2)cc1 ; Clc1ccc(-c2cnc(Cl)o2)cc1 ; N#Cc1ccc(N)cn1 ; N#Cc1ccc(Nc2ncc(-c3ccc(Cl)cc3)o2)cn1 ; N/C(=N\O)c1ccc(Nc2ncc(-c3ccc(Cl)cc3)o2)cn1]

5-(4-Chlorophenyl)oxazole (5.0 g, 27.8 mmol) was reacted with LiHMDS (33.4 mL, 33.4 mmol) then C₂Cl₆ (9.9 g, 41.8 mmol) in dry THF as per General Procedure 15 Method 1 to give 2-chloro-5-(4-chlorophenyl)oxazole (5.1 g, 86% yield) as a white solid. ¹H NMR (401 MHz, CDCl₃) δ 7.57-7.50 (m, 2H), 7.44-7.38 (m, 2H), 7.28 (s, 1H). LCMS R_{f} (min)=3.265, MS m/z=214.0 [M+H]⁺.

5-Aminonicotinonitrile (200 mg, 1.68 mmol, 1.0 eq.), NaH (60%, 60 mg, 2.52 mmol, 1.5 eq.) and 2-chloro-5-(4-chlorophenyl)oxazole (431 mg, 2.01 mmol, 1.2 eq.) in anhydrous DMF (4 mL) was reacted according to General Procedure 2 Method 2 to afford 5-((5-(4-chlorophenyl)oxazol-2-yl)amino)picolinonitrile as a beige solid (186 mg, 37%). ¹H NMR (401 MHz, DMSO-d₆) δ 8.82 (d, J=2.4 Hz, 1H), 8.31 (dd, J=8.7, 2.6 Hz, 1H), 7.98 (d, J=8.8 Hz, 1H), 7.66-7.60 (m, 3H), 7.54-7.48 (m, 2H), ¹³C NMR (101 MHz, DMSO-d₆) δ 155.21, 144.11, 140.19, 139.20, 131.99, 129.94, 129.27, 126.51, 124.69, 123.72, 123.37, 122.68, 118.15. LCMS R_{f}(min)=3.039, MS m/z=297.0 [M+H]⁺.

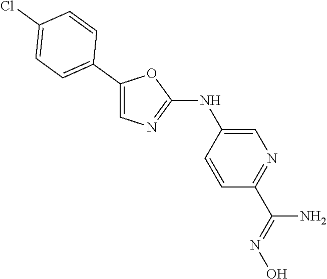 [SMILES: N/C(=N\O)c1ccc(Nc2ncc(-c3ccc(Cl)cc3)o2)cn1]

5-((5-(4-Chlorophenyl)oxazol-2-yl)amino)picolinonitrile (50 mg, 0.169 mmol, 1.0 eq.), NH₂OH·HCl (47 mg, 0.674 mmol, 4.0 eq.) and Et₃N (94 μL, 0.674 mmol, 4.0 eq.) in anhydrous MeOH (2 mL) was reacted according to General Procedure 1 Method 2 to afford 5-((5-(4-chlorophenyl)oxazol-2-yl)amino)-N′-hydroxypicolinimidamide as a beige solid (48 mg, 86%). ¹H NMR (401 MHz, DMSO-d₆) δ 10.80 (s, 1H), 9.75 (s, 1H), 8.76 (d, J=2.5 Hz, 1H), 8.11 (dd, J=8.8, 2.6 Hz, 1H), 7.82 (d, J=8.8 Hz, 1H), 7.62 (d, J=8.6 Hz, 2H), 7.56 (s, 1H), 7.50 (d, J=8.6 Hz, 2H), 5.76 (s, 2H), ¹³C NMR (101 MHz, DMSO-d₆) δ 156.06, 149.48, 143.43, 142.98, 136.76, 136.34, 131.57, 129.14, 126.73, 124.41, 123.82, 123.37, 119.74. LCMS R_{f} (min)=2.446, MS m/z=330.1 [M+H]⁺. HRMS (ESI) calcd for C₁₅H₁₃ClN₅O₂⁺ ([M+H]⁺ 330.0752, found 330.0764.

### 101. 5-((5-(4-Chlorophenyl)oxazol-2-yl)amino)-N′-hydroxypyrazine-2-carboximidamide (Scheme 98)

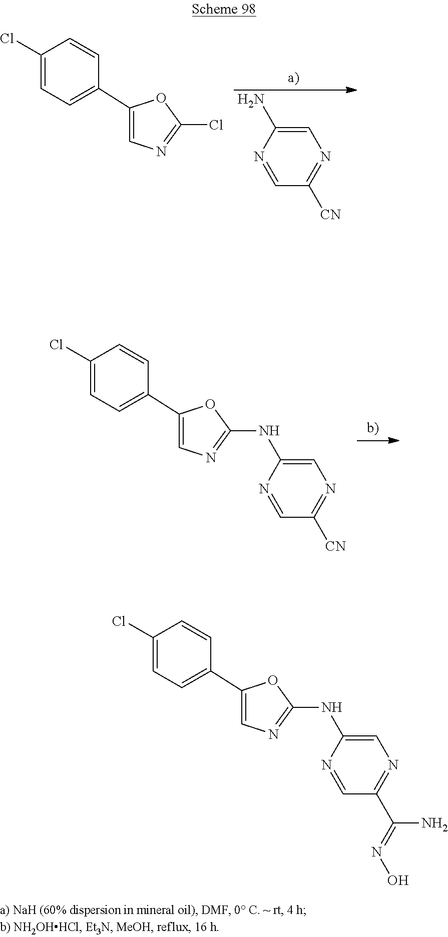 [SMILES: Clc1ccc(-c2cnc(Cl)o2)cc1 ; N#Cc1cnc(N)cn1 ; N#Cc1cnc(Nc2ncc(-c3ccc(Cl)cc3)o2)cn1 ; N/C(=N\O)c1cnc(Nc2ncc(-c3ccc(Cl)cc3)o2)cn1]

5-Aminopyrazine-2-carbonitrile (200 mg, 1.67 mmol, 1.0 eq.), NaH (60%, 60 mg, 2.50 mmol, 1.5 eq.) and 2-chloro-5-(4-chlorophenyl)oxazole (428 mg, 2.00 mmol, 1.2 eq.) in anhydrous DMF (4 mL) was reacted according to General Procedure 2 Method 2 to afford 5-((5-(4-chlorophenyl)oxazol-2-yl)amino)pyrazine-2-carbonitrile as a yellow solid (355 mg, 72%). ¹H NMR (401 MHz, DMSO-d₆) δ 9.21 (s, 1H), 8.85 (d, J=1.3 Hz, 1H), 7.69 (s, 1H), 7.68-7.63 (m, 2H), 7.56-7.51 (m, 2H); ¹³C NMR (101 MHz, DMSO-d₆) δ 154.23, 147.62, 132.33, 129.28, 126.31, 124.91, 120.39, 117.00, 113.95 ppm. LCMS R_{f}(min)=3.010, MS m/z=298.0 [M+H]⁺.

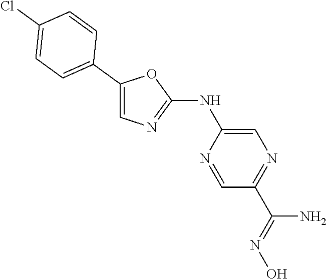 [SMILES: N/C(=N\O)c1cnc(Nc2ncc(-c3ccc(Cl)cc3)o2)cn1]

5-((5-(4-Chlorophenyl)oxazol-2-yl)amino)pyrazine-2-carbonitrile (50 mg, 0.168 mmol, 1.0 eq.), NH₂OH·HCl (47 mg, 0.672 mmol, 4.0 eq.) and Et₃N (94 L, 0.672 mmol, 4.0 eq.) in anhydrous MeOH (2 mL) was reacted according to General Procedure 1 Method 2 to afford 5-((5-(4-chlorophenyl)oxazol-2-yl)amino)-N′-hydroxypyrazine-2-carboximidamide as a white solid (43 mg, 77%). ¹H NMR (401 MHz, DMSO-d₆) δ 9.09 (s, 1H), 8.38 (s, 1H), 7.89 (s, 1H), 6.92-6.73 (m, 3H), 6.68 (d, J=8.3 Hz, 2H), 5.01 (s, 2H); ¹³C NMR (101 MHz, DMSO-d₆) δ 154.92, 148.36, 139.30, 138.91, 131.82, 129.17, 126.55, 124.57, 123.35. LCMS R_{f}(min)=2.537, MS m/z=331.0 [M+H]⁺. HRMS (ESI) calcd for C₁₄H₁₂ClN₆O₂⁺ ([M+H]⁺ 331.0705, found 331.0704.

### 102. 5-((5-(5-Chloropyridin-2-yl)oxazol-2-yl)amino)-N′-hydroxypyrazine-2-carboximidamide (Scheme 99)

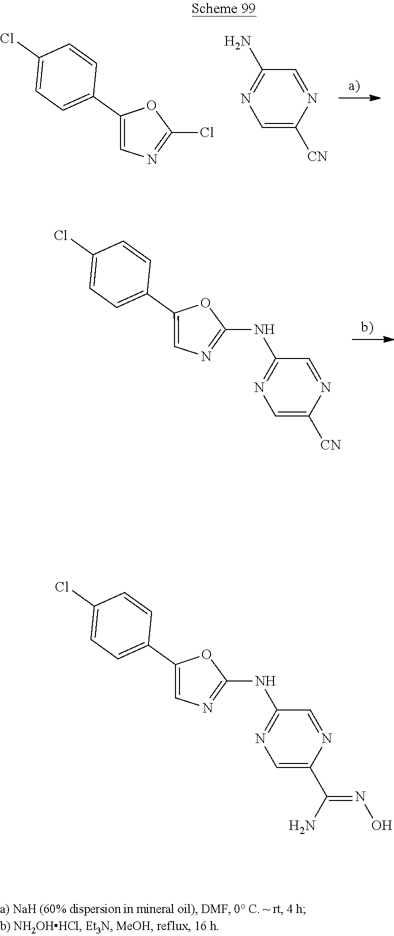 [SMILES: Clc1ccc(-c2cnc(Cl)o2)cc1 ; N#Cc1cnc(N)cn1 ; N#Cc1cnc(Nc2ncc(-c3ccc(Cl)cc3)o2)cn1 ; N/C(=N\O)c1cnc(Nc2ncc(-c3ccc(Cl)cc3)o2)cn1]

5-Aminopyrazine-2-carbonitrile (279 mg, 2.33 mmol, 1.2 eq.), NaH (60%, 84 mg, 3.49 mmol, 3.0 eq.) and 2-chloro-5-(5-chloropyridin-2-yl)oxazole (250 mg, 1.16 mmol, 1.0 eq.) in anhydrous DMF (5 mL) was reacted according to General Procedure 2 Method 2 to afford 5-((5-(5-chloropyridin-2-yl)oxazol-2-yl)amino)pyrazine-2-carbonitrile as a yellow solid (232 mg, 67%). ¹H NMR (401 MHz, DMSO-d₆) δ 9.23 (s, 1H), 8.83 (s, 1H), 8.64 (d, J=2.3 Hz, 1H), 8.07-7.97 (m, 1H), 7.79 (s, 1H), 7.67 (d, J=8.6 Hz, 1H). LCMS R_{f} (min)=2.717, MS m/z=299.1 [M+H]⁺.

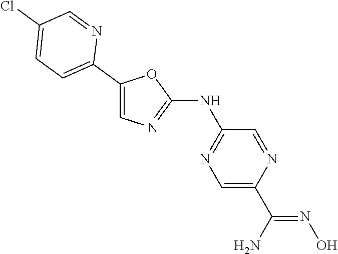 [SMILES: N/C(=N\O)c1cnc(Nc2ncc(-c3ccc(Cl)cn3)o2)cn1]

5-((5-(5-Chloropyridin-2-yl)oxazol-2-yl)amino)pyrazine-2-carbonitrile (100 mg, 0.336 mmol, 1.0 eq.), NH₂OH·HCl (93 mg, 1.34 mmol, 4.0 eq.) and Et₃N (187 L, 1.34 mmol, 4.0 eq.) in anhydrous MeOH (5 mL) was reacted according to General Procedure 1 Method 2 to afford 5-((5-(5-chloropyridin-2-yl)oxazol-2-yl)amino)-N′-hydroxypyrazine-2-carboximidamide as a beige solid (91 mg, 81%). ¹H NMR (401 MHz, DMSO-d₆) δ 9.96 (s, 1H), 9.25 (s, 1H), 8.75 (m, 1H), 8.63 (d, J=2.4 Hz, 1H), 8.02 (dd, J=8.5, 2.5 Hz, 1H), 7.77 (s, 1H), 7.66 (d, J=8.5 Hz, 1H), 5.86 (s, 2H). LCMS R_{f}(min)=2.423, MS m/z=332.1 [M+H]⁺. HRMS (ESI) calcd for C₁₃H₁₁ClN₇O₂⁺ ([M+H]⁺ 332.0657, found 332.0666.

### 103. 5-((1-(5-Chloropyridin-2-yl)-1H-pyrazol-4-yl)amino)-N′-hydroxypicolinimidamide (Scheme 100)

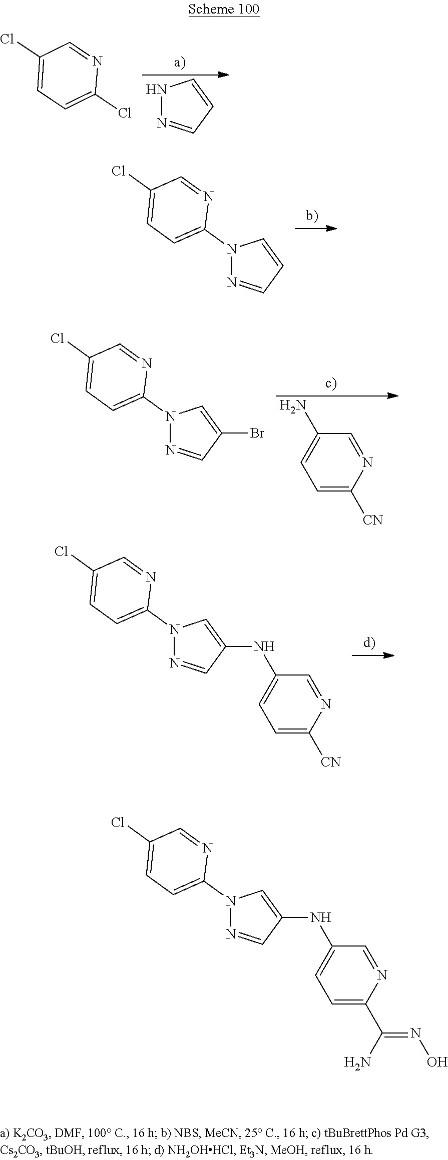

To a solution of 2,5-dichloropyridine (3.0 g, 20.3 mmol, 1.0 eq.) in DMF (25 mL), was added K₂CO₃ (7.0 g, 50.7 mmol, 2.5. eq.) and 1H-pyrazole (2.76 g, 40.5 mmol, 2.0 eq.), and the mixture was allowed to stir at 100° C. for 16 h. Upon cooling, the reaction mixture was concentrated in vacuo and purified via column chromatography (petroleum spirits, 0-20% EtOAc) to give 5-chloro-2-(1H-pyrazol-1-yl)pyridine as a white solid (3.21 g, 88%). ¹H NMR (401 MHz, DMSO-d₆) δ 8.56 (dd, J=2.6, 0.6 Hz, 1H), 8.51-8.47 (m, 1H), 8.07 (dd, J=8.8, 2.6 Hz, 1H), 7.92 (dd, J=8.8, 0.5 Hz, 1H), 7.83 (dd, J=1.5, 0.5 Hz, 1H), 6.58 (dd, J=2.6, 1.7 Hz, 1H). LCMS R_{f}(min)=2.992, MS m/z=180.1 [M+H]⁺.

To a solution of 5-chloro-2-(1H-pyrazol-1-yl)pyridine (3.0 g, 16.7 mmol, 1.0 eq.) in MeCN (25 mL) was added N-bromosuccinimide (4.46 g, 25.1 mmol, 1.5 eq.), and the mixture was allowed to stir at 25° C. for 16 h. Upon completion, the reaction mixture was concentrated in vacuo and purified via reverse-phase column chromatography (H₂O, MeCN 10-100%) to give 2-(4-bromo-1H-pyrazol-1-yl)-5-chloropyridine as a white solid (499 mg, 12%). ¹H NMR (401 MHz, DMSO-d₆) δ 8.82-8.75 (m, 1H), 8.55 (d, J=2.3 Hz, 1H), 8.13 (dd, J=8.8, 2.6 Hz, 1H), 7.99 (s, 1H), 7.92 (d, J=8.8 Hz, 1H). ¹³C NMR (101 MHz, DMSO-d₆) δ 148.80, 146.82, 142.96, 139.59, 129.02, 127.62, 113.26, 96.42. LCMS R_{f} (min)=3.396, MS m/z=258.0 [M+H]⁺.

2-(4-Bromo-1H-pyrazol-1-yl)-5-chloropyridine (150 mg, 0.58 mmol, 1.0 eq.), 5-aminonicotinonitrile (104 mg, 0.87 mmol, 1.5 eq.), Cs₂CO₃ (567 mg, 1.74 mmol, 3.0 eq.) in tBuOH was charged with tBuBrettPhos Pd G3 (25 mg, 0.029 mmol, 0.05 eq.) and was reacted according to General Procedure 4 Method 2 to afford 5-((1-(5-chloropyridin-2-yl)-1H-pyrazol-4-yl)amino)picolinonitrile as a brown solid (105 mg, 61%). ¹H NMR (401 MHz, DMSO-d₆) δ 9.14 (s, 1H), 8.65-8.48 (m, 2H), 8.28 (d, J=2.3 Hz, 1H), 8.10 (dd, J=8.8, 2.2 Hz, 1H), 7.93 (m, 2H), 7.76 (d, J=8.7 Hz, 1H), 7.34 (dd, J=8.6, 2.5 Hz, 1H). LCMS R_{f}(min)=2.898, MS m/z=297.0 [M+H]⁺

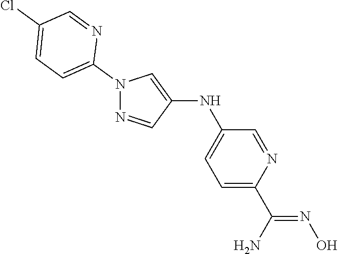 [SMILES: N/C(=N\O)c1ccc(Nc2cnn(-c3ccc(Cl)cn3)c2)cn1]

5-((1-(5-Chloropyridin-2-yl)-1H-pyrazol-4-yl)amino)picolinonitrile (105 mg, 0.353 mmol, 1.0 eq.), NH₂OH·HCl (98 mg, 1.41 mmol, 4.0 eq.) and Et₃N (197 L, 1.41 mmol, 4.0 eq.) in anhydrous MeOH (5 mL) was reacted according to General Procedure 1 Method 2 to afford 5-((1-(5-chloropyridin-2-yl)-1H-pyrazol-4-yl)amino)-N′-hydroxypicolinimidamide as a beige solid (70 mg, 60%). ¹H NMR (401 MHz, DMSO-d₆) δ 9.57 (s, 1H), 8.57-8.51 (m, 2H), 8.47 (s, 1H), 8.19 (d, J=2.6 Hz, 1H), 8.09 (dd, J=8.8, 2.5 Hz, 1H), 7.92 (d, J=8.9 Hz, 1H), 7.87 (s, 1H), 7.71 (d, J=8.7 Hz, 1H), 7.34 (dd, J=8.7, 2.7 Hz, 1H), 5.67 (s, 2H). ¹³C NMR (101 MHz, DMSO-d₆) δ 149.33, 149.17, 146.31, 141.34, 139.50, 138.85, 136.35, 134.07, 127.48, 126.98, 119.82, 119.70, 115.27, 112.51. LCMS R_{f}(min)=2.271, MS m/z=330.1 [M+H]⁺. HRMS (ESI) calcd for C₁₄H₁₃C₁N₇O⁺ ([M+H]⁺ 330.0865, found 330.0872.

### 104. rac-N-(2,3-Dihydroxypropyl)-5-((1-(5-(trifluoromethyl)pyridin-2-yl)-1H-pyrazol-4-yl)amino)picolinamide (Scheme 101)

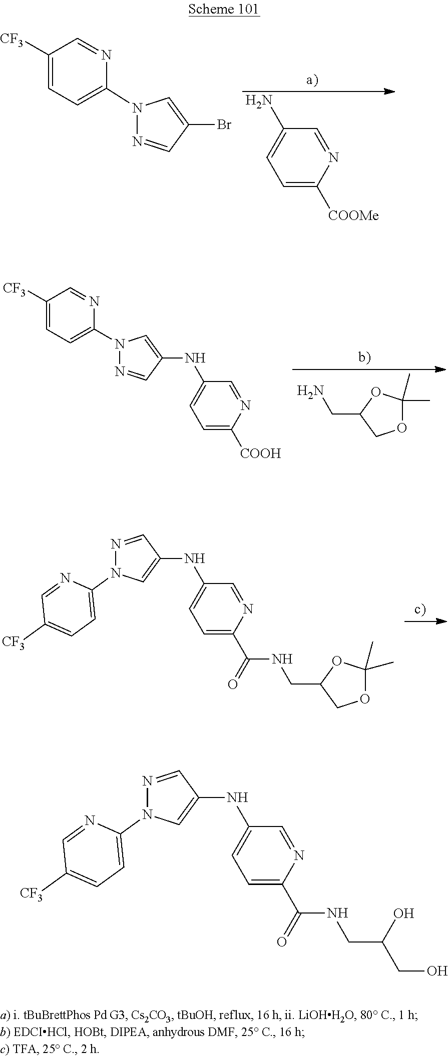

A solution of 2-(4-bromo-1H-pyrazol-1-yl)-5-(trifluoromethyl)pyridine (Intermediate M, 250 mg, 0.856 mmol, 1.0 eq.), methyl 5-aminopicolinate (153 mg, 1.28 mmol, 1.5 eq.), Cs₂CO₃ (837 mg, 2.57 mmol, 3.0 eq.) in tBuOH (5 mL) was charged with tBuBrettPhos Pd G3 (37 mg, 0.043 mmol, 0.05 eq.) and the mixture was subjected to three cycles of evacuation and backfilling with N₂. The reaction mixture was heated to refluxed and stirred for 16 h. Upon cooling, LiOH·H₂O (108 mg, 2.57 mmol, 3 eq.) in H₂O (1.5 mL) was added and the mixture was stirred at 80° C. for 1 h. The suspension was acidified with concentrated HCl to pH 1, at which precipitation was observed. The precipitate was filtered and washed with H₂O (15 mL) to yield the crude material, which was purified via reverse-phase column chromatography (H₂O, MeCN 10-100%) to afford 5-((1-(5-(trifluoromethyl)pyridin-2-yl)-1H-pyrazol-4-yl)amino)picolinic acid as a yellow solid (108 mg, 38%). ¹H NMR (401 MHz, DMSO-d₆) δ 9.00 (s, 1H), 8.88 (d, J=0.8 Hz, 1H), 8.63 (s, 1H), 8.37 (dd, J=8.8, 2.3 Hz, 1H), 8.30 (d, J=2.6 Hz, 1H), 8.09 (d, J=8.7 Hz, 1H), 8.00 (s, 1H), 7.93 (d, J=8.7 Hz, 1H), 7.39 (dd, J=8.7, 2.8 Hz, 1H). LCMS R_{f}(min)=2.541, MS m/z=350.1 [M+H]⁺

To a solution of 5-((1-(5-(trifluoromethyl)pyridin-2-yl)-1H-pyrazol-4-yl)amino)picolinic acid (80 mg, 0.229 mmol, 1.0 eq.), EDCI·HCl (57 mg, 0.298 mmol, 1.3 eq.), HOBt (43 mg, 0.321 mmol, 1.4 eq.), DIPEA (79 μL, 0.458 mmol, 2.0 eq.) in anhydrous DMF (5 mL) was added (2,2-dimethyl-1,3-dioxolan-4-yl)methanamine hydrochloride (60 mg, 0.458 mmol, 2.0 eq.). The reaction was allowed to stir at 25° C. for 16 h. Upon completion, the reaction was added to a mixture of ice-water (25 mL), at which precipitation was observed. The precipitate was filtered to afford rac-N-((2,2-dimethyl-1,3-dioxolan-4-yl)methyl)-5-((1-(5-(trifluoromethyl)pyridin-2-yl)-1H-pyrazol-4-yl)amino)picolinamide as orange solid (89 mg, 84%). ¹H NMR (401 MHz, DMSO-d₆) δ 8.88 (dd, J=6.6, 5.3 Hz, 2H), 8.61 (d, J=0.6 Hz, 1H), 8.43 (t, J=6.1 Hz, 1H), 8.36 (dd, J=8.8, 2.3 Hz, 1H), 8.24 (d, J=2.7 Hz, 1H), 8.09 (d, J=8.7 Hz, 1H), 7.98 (d, J=0.7 Hz, 1H), 7.90 (d, J=8.6 Hz, 1H), 7.43 (dd, J=8.6, 2.8 Hz, 1H), 4.30-4.16 (m, 1H), 3.97 (dd, J=8.3, 6.4 Hz, 1H), 3.70 (dd, J=8.3, 5.7 Hz, 1H), 3.41 (d, J=6.0 Hz, 2H), 1.35 (s, 3H), 1.26 (s, 3H); ¹³C NMR (101 MHz, DMSO-d₆) δ 164.19, 153.20, 145.91, 143.41, 139.45, 137.84, 136.98, 135.16, 127.01, 123.23, 122.66, 122.34, 119.02, 116.34, 111.64, 108.43, 74.25, 66.67, 41.49, 26.82, 25.28. LCMS R_{f}(min)=2.978, MS m/z=463.2 [M+H]⁺

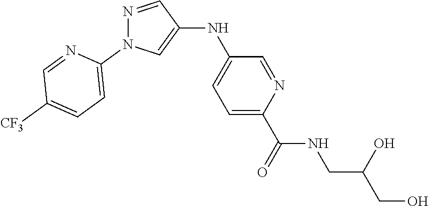 [SMILES: O=C(NCC(O)CO)c1ccc(Nc2cnn(-c3ccc(C(F)(F)F)cn3)c2)cn1]

rac-N-((2,2-Dimethyl-1,3-dioxolan-4-yl)methyl)-5-((1-(5-(trifluoromethyl)pyridin-2-yl)-1H-pyrazol-4-yl)amino)picolinamide (70 mg, 0.151 mmol, 1.0 eq.) was stirred in TFA (2 mL) at 25° C. for 2 h. Upon completion, the reaction was concentrated in vacuo and subjected to purification via reverse-phase column chromatography (H₂O, MeCN 10-100%) to afford rac-N-(2,3-dihydroxypropyl)-5-((1-(5-(trifluoromethyl)pyridin-2-yl)-1H-pyrazol-4-yl)amino)picolinamide as a beige solid (18 mg, 28%). ¹H NMR (401 MHz, DMSO-d₆) δ 8.94-8.84 (m, 2H), 8.61 (s, 1H), 8.36 (dd, J=8.7, 2.1 Hz, 1H), 8.31 (t, J=5.9 Hz, 1H), 8.25 (d, J=2.7 Hz, 1H), 8.09 (d, J=8.7 Hz, 1H), 7.98 (s, 1H), 7.90 (d, J=8.6 Hz, 1H), 7.44 (dd, J=8.6, 2.7 Hz, 1H), 4.93 (d, J=4.9 Hz, 1H), 4.65 (t, J=5.7 Hz, 1H), 3.65-3.54 (m, 1H), 3.54-3.43 (m, 1H), 3.24-3.12 (m, 1H) *Resonances that overlapped with H₂O were not assigned; ¹³C NMR (101 MHz, DMSO-d₆) δ 164.06, 153.20, 145.92, 143.33, 139.93, 139.62, 137.81, 136.98, 135.18, 127.06, 123.10, 122.66, 119.07, 116.27, 111.65, 70.32, 63.93, 42.23. LCMS R_{f} (min)=2.566, MS m/z=423.1 [M+H]⁺. HRMS (ESI) calcd for C₁₈H₁₈F₃N₆O₃⁺ ([M+H]⁺ 423.1387, found 423.1399.

### 105. 6-((5-(4-(Trifluoromethoxy)phenyl)oxazol-2-yl)amino)pyridin-3-ol (Scheme 102)

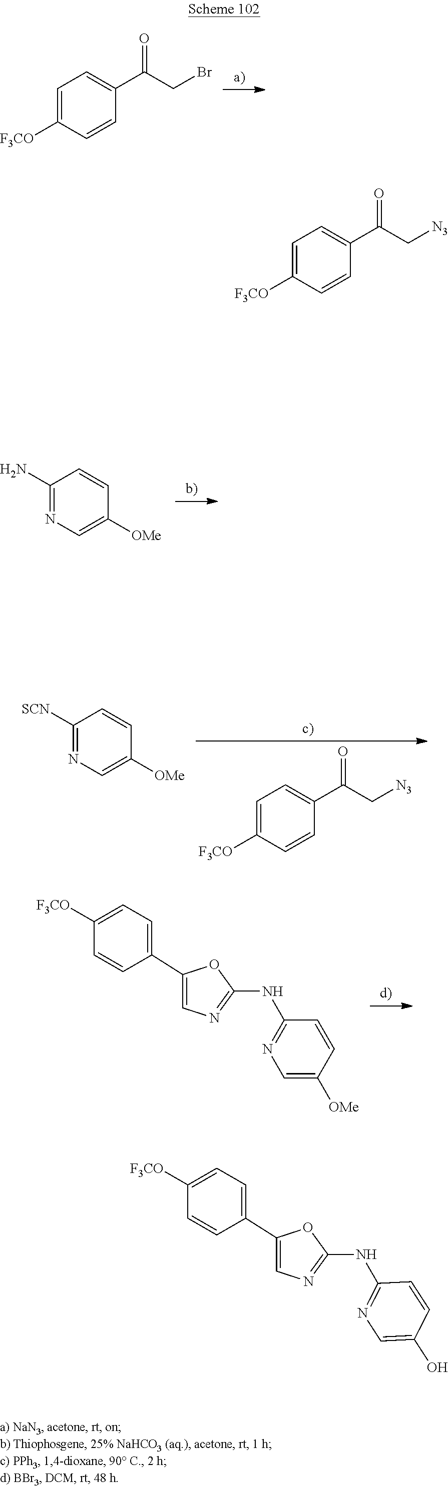

A solution of 2-bromo-1-(4-(trifluoromethoxy)phenyl)ethan-1-one (0.51 g, 1.80 mmol) and NaN₃ (0.23 g, 3.59 mmol) in acetone (9 mL) was stirred for 18 h at rt. The reaction mixture was then concentrated in vacuo and the residue was redissolved in EtOAc (30 mL), washed with H₂O (15 mL) and brine (15 mL). The organic layer was dried over MgSO₄ and concentrated under reduced pressure to give 2-azido-1-(4-(trifluoromethoxy)phenyl)ethan-1-one as a yellow solid (0.41 g, 92%). ¹H NMR (401 MHz, CDCl₃) δ 7.98 (d, J=9.0 Hz, 2H), 7.33 (m, 2H), 4.54 (s, 2H).

5-Methoxypyridin-2-amine (0.33 g, 2.63 mmol) was reacted with thiophosgene (0.24 mL, 3.15 mmol) as per General Procedure 6. The suspension was allowed to stir for 1 h at rt to give 2-isothiocyanato-5-methoxypyridine as an orange wax (0.24 g, 54% yield). ¹H NMR (401 MHz, CDCl₃) δ 8.09 (d, J=2.8 Hz, 1H), 7.21 (dd, J=8.7, 3.1 Hz, 1H), 7.07 (m, 1H), 3.87 (s, 3H).

2-Azido-1-(4-(trifluoromethoxy)phenyl)ethan-1-one (0.20 g, 0.83 mmol) and 2-isothiocyanato-5-methoxypyridine (0.15 g, 0.92 mmol) were reacted as per General Procedure 5 for 2.5 h. The reaction mixture was then concentrated to a solid and triturated with DCM to give N-(5-methoxypyridin-2-yl)-5-(4-(trifluoromethoxy)phenyl)oxazol-2-amine (0.15 g, 51% yield) as a white solid. ¹H NMR (401 MHz, DMSO-d₆) δ 10.73 (s, 1H), 8.01 (m, 1H), 7.98 (d, J=9.1 Hz, 1H), 7.69 (d, J=8.9 Hz, 2H), 7.54 (s, 1H), 7.49-7.41 (m, 3H), 3.80 (s, 3H). LCMS R_{f} (min)=2.97, MS m/z=352.1 [M+H]⁺.

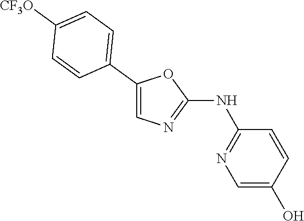

N-(5-Methoxypyridin-2-yl)-5-(4-(trifluoromethoxy)phenyl)oxazol-2-amine (0.10 g, 0.29 mmol) was demethylated as per General Procedure 8 using BBr₃ (1.0 M in DCM, 0.9 mL, 0.88 mmol) in dry DCM (1.5 mL) and the reaction mixture was allowed to stir for 48 h at rt. The crude residue was purified through C₁₈ reverse phase column chromatography to give 6-((5-(4-(trifluoromethoxy)phenyl)oxazol-2-yl)amino)pyridin-3-ol (0.04 g, 38%) as an off-white solid. ¹H NMR (401 MHz, DMSO-d₆) δ 10.56 (s, 1H), 9.48 (s, 1H), 7.87 (d, J=8.9 Hz, 1H), 7.84 (d, J=2.9 Hz, 1H), 7.68 (d, J=8.9 Hz, 2H), 7.51 (s, 1H), 7.43 (d, J=8.1 Hz, 2H), 7.23 (dd, J=8.9, 3.0 Hz, 1H). ¹³C NMR (101 MHz, DMSO-d₆) δ 156.6, 149.0, 146.9, 144.5, 142.7, 135.0, 127.4, 125.0, 124.3, 123.5, 121.80, 111.6. LCMS R_{f} (min)=2.65, MS m/z=338.1 [M+H]⁺·HRMS (ESI) calcd for C₁₅H₁₁F₃N₃O₃⁺ [M+H]⁺ 338.0747, found 338.0757.

### 106. N′-Hydroxy-5-((1-(4-(trifluoromethyl)phenyl)-1H-1,2,4-triazol-3-yl)amino)pyrazine-2-carboximidamide (Scheme 103)

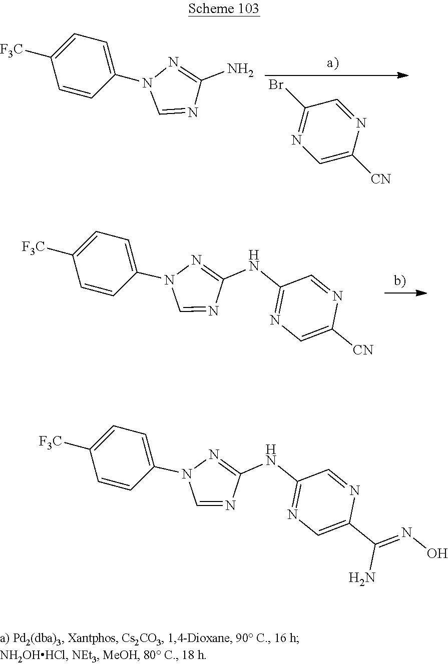 [SMILES: Nc1ncn(-c2ccc(C(F)(F)F)cc2)n1 ; N#Cc1cnc(Br)cn1 ; N#Cc1cnc(Nc2ncn(-c3ccc(C(F)(F)F)cc3)n2)cn1 ; N/C(=N\O)c1cnc(Nc2ncn(-c3ccc(C(F)(F)F)cc3)n2)cn1]

Intermediate F (0.07 g, 0.33 mmol) and 5-bromopyrazine-2-carbonitrile (0.05 g, 0.27 mmol) were reacted as per General Procedure 4 Method 1, using 5 mol % Pd₂(dba)₃, 10 mol % Xantphos, 1.5 eq Cs₂CO₃ and heated at 90° C. for 16 h. The crude residue was purified as described in General Procedure 4 Method 1 to give 5-((1-(4-(trifluoromethyl)phenyl)-1H-1,2,4-triazol-3-yl)amino)pyrazine-2-carbonitrile (0.07 g, 80% yield) as an orange solid. ¹H NMR (401 MHz, DMSO-d₆) δ 11.39 (s, 1H), 9.41 (s, 1H), 9.24 (d, J=1.1 Hz, 1H), 8.80 (d, J=1.4 Hz, 1H), 8.12 (d, J=8.5 Hz, 2H), 7.96 (d, J=8.6 Hz, 2H). LCMS R_{f} (min)=2.90, MS m/z=332.0 [M+H]⁺.

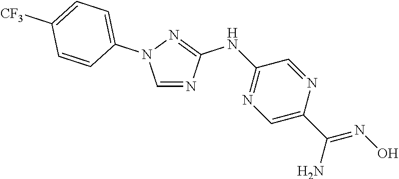 [SMILES: N/C(=N\O)c1cnc(Nc2ncn(-c3ccc(C(F)(F)F)cc3)n2)cn1]

5-((1-(4-(Trifluoromethyl)phenyl)-1H-1,2,4-triazol-3-yl)amino)pyrazine-2-carbonitrile (0.06 g, 0.18 mmol) was reacted as per General Procedure 1 Method 2 using NH₂OH·HCl (0.05 g, 0.70 mmol) and NEt₃ (0.1 mmol) in dry MeOH (1 mL) to give N′-hydroxy-5-((1-(4-(trifluoromethyl)phenyl)-1H-1,2,4-triazol-3-yl)amino)pyrazine-2-carboximidamide (0.04 g, 67% yield) as a pink solid. ¹H NMR (401 MHz, DMSO-d₆) δ 10.70 (s, 1H), 9.89 (s, 1H), 9.36 (s, 1H), 9.25 (d, J=1.1 Hz, 1H), 8.70 (d, J=1.2 Hz, 1H), 8.11 (d, J=8.4 Hz, 2H), 7.96 (d, J=8.6 Hz, 2H), 5.84 (s, 2H). ¹³C NMR (101 MHz, DMSO-d₆) δ 159.2, 149.88, 148.5, 139.5, 139.3, 137.7, 130.7, 127.1, 118.8. LCMS R_{f}(min)=2.44. MS m/z=365.1 [M+H]⁺. HRMS (ESI) calcd for C₁₄H₁₂F₃N₈O⁺ [M+H]⁺ 365.1081, found 365.1100.

### 107. N′-Hydroxy-5-((1-(5-(trifluoromethyl)pyridin-2-yl)-1H-1,2,4-triazol-3-yl)amino)picolinimidamide (Scheme 104)

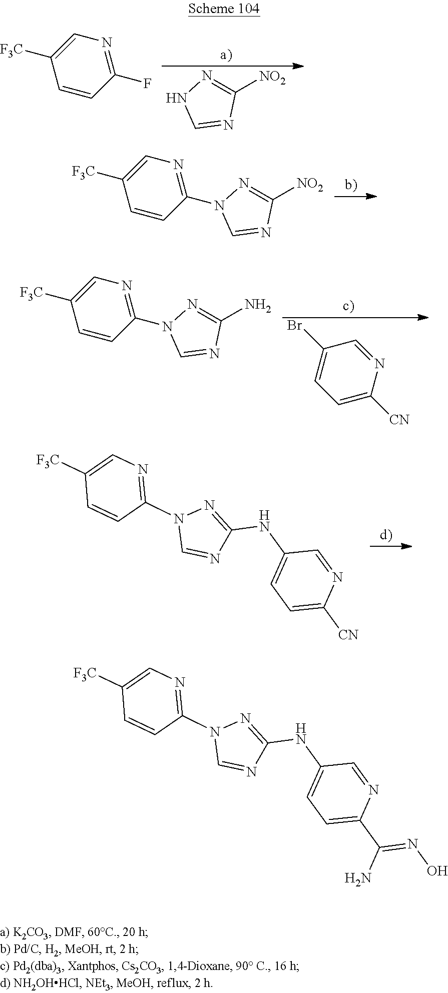

A suspension of 2-fluoro-5-(trifluoromethyl)pyridine (3.0 g, 18.17 mmol), 3-nitro-1H-1,2,4-triazole (3.11 g, 27.26 mmol) and K₂CO₃ (6.28 g, 45.43 mmol) in DMF (90 mL) was heated to 60° C. and allowed to stir for 20 h. The reaction mixture was diluted with EtOAc (2×100 mL), washed with H₂O (100 mL) and brine (100 mL). The organic layer was dried over MgSO₄ and concentrated under reduced pressure. The resultant crude residue was purified by flash chromatography, eluting with 20-30% EtOAc in petroleum spirit to give 2-(3-nitro-1H-1,2,4-triazol-1-yl)-5-(trifluoromethyl)pyridine (2.50 g, 53% yield) as a white solid. ¹H NMR (401 MHz, DMSO-d₆) δ 9.79 (s, 1H), 9.08 (dd, J=1.5, 0.8 Hz, 1H), 8.58 (ddd, J=8.7, 2.4, 0.5 Hz, 1H), 8.17 (d, J=8.6 Hz, 1H). LCMS R_{f}(min)=2.85, MS m/z=260.1 [M+H]⁺.

A solution of 2-(3-nitro-1H-1,2,4-triazol-1-yl)-5-(trifluoromethyl)pyridine (1.50 g, 5.79 mmol) in MeOH (50 mL) was evacuated and backfilled with N₂×3, then 10% Pd/C (0.75 g) was added. The suspension was evacuated and flushed with H₂×3 and stirred under a hydrogen atmosphere (balloon) for 2 h. The reaction mixture was then filtered through a pad of celite and the filtrate was concentrated under reduced pressure to give 1-(5-(trifluoromethyl)pyridin-2-yl)-1H-1,2,4-triazol-3-amine (0.78 g, 59% yield) as a black solid. ¹H NMR (401 MHz, DMSO-d₆) δ 8.99 (s, 1H), 8.84 (dd, J=1.5, 0.8 Hz, 1H), 8.37 (dd, J=8.7, 2.0 Hz, 1H), 7.74 (d, J=8.6 Hz, 1H), 6.05 (s, 2H). LCMS R_{f}(min)=2.60. MS m/z=230.1 [M+H]⁺.

1-(5-(Trifluoromethyl)pyridin-2-yl)-1H-1,2,4-triazol-3-amine (0.05 g, 0.22 mmol) and 5-bromopicolinonitrile (0.04 g, 0.20 mmol) were reacted as per General Procedure 4 Method 1, using 5 mol % Pd₂(dba)₃, 10 mol % Xantphos, 1.5 eq Cs₂CO₃ and heated at 90° C. for 16 h. The crude residue was purified as per General Procedure 4 Method 1 to give 5-((1-(5-(trifluoromethyl)pyridin-2-yl)-1H-1,2,4-triazol-3-yl)amino)picolinonitrile (0.04 g, 65% yield) as a grey solid. ¹H NMR (401 MHz, DMSO-d₆) δ 10.70 (s, 1H), 9.39 (s, 1H), 8.94 (s, 1H), 8.89 (d, J=2.6 Hz, 1H), 8.48 (dd, J=8.7, 2.3 Hz, 1H), 8.32 (dd, J=8.7, 2.6 Hz, 1H), 8.07 (d, J=8.5 Hz, 1H), 7.97 (d, J=8.7 Hz, 1H); LCMS R_{f}(min)=2.91. MS m/z=332.1 [M+H]⁺.

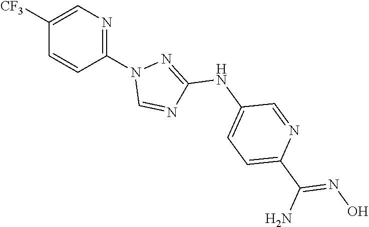 [SMILES: N/C(=N\O)c1ccc(Nc2ncn(-c3ccc(C(F)(F)F)cn3)n2)cn1]

5-((1-(5-(Trifluoromethyl)pyridin-2-yl)-1H-1,2,4-triazol-3-yl)amino)picolinonitrile (0.05 g, 0.16 mmol) was reacted as per General Procedure 1 Method 3 using NH₂OH·HCl (0.04 g, 0.63 mmol) and NEt₃ (0.09 mL, 0.63 mmol) in dry MeOH (1.0 mL). The resultant crude residue was then purified by C18 reverse phase column chromatography to give N′-hydroxy-5-((1-(5-(trifluoromethyl)pyridin-2-yl)-1H-1,2,4-triazol-3-yl)amino)picolinimidamide (0.02 g, 40%) as a white solid. ¹H NMR (401 MHz, DMSO-d₆) δ 10.14 (s, 1H), 9.69 (s, 1H), 9.32 (s, 1H), 8.92 (s, 1H), 8.87 (d, J=2.4 Hz, 1H), 8.46 (dd, J=8.7, 2.1 Hz, 1H), 8.10 (dd, J=8.8, 2.6 Hz, 1H), 8.04 (d, J=8.6 Hz, 1H), 7.82 (d, J=8.8 Hz, 1H), 5.74 (s, 2H); ¹³C NMR (101 MHz, DMSO-d₆) δ 160.8, 149.5, 141.9, 137.9, 136.8, 123.4, 119.6, 112.2; LCMS R_{f} (min)=3.13, MS m/z=365.1 [M+H]⁺. HRMS (ESI) calcd for C₁₄H₁₂F₃N₈O [M+H]⁺ 365.1081, found 365.1093.

### 108. N′-Hydroxy-5-((1-(5-(trifluoromethyl)pyridin-2-yl)-1H-1,2,4-triazol-3-yl)amino)pyrazine-2-carboximidamide (Scheme 105)

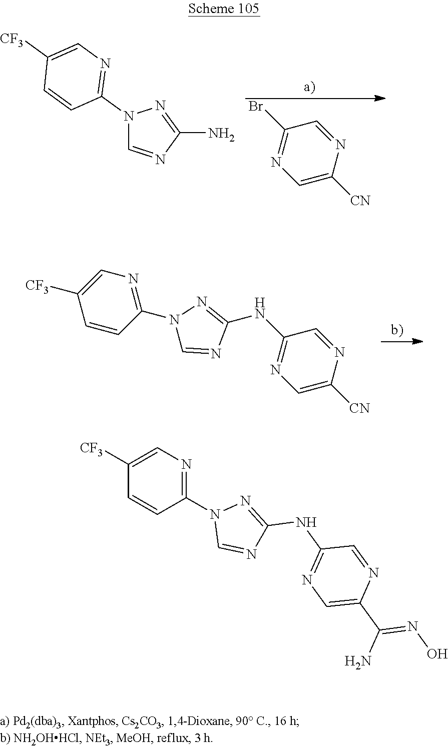 [SMILES: Nc1ncn(-c2ccc(C(F)(F)F)cn2)n1 ; N#Cc1cnc(Br)cn1 ; N#Cc1cnc(Nc2ncn(-c3ccc(C(F)(F)F)cn3)n2)cn1 ; N/C(=N\O)c1cnc(Nc2ncn(-c3ccc(C(F)(F)F)cn3)n2)cn1]

1-(5-(Trifluoromethyl)pyridin-2-yl)-1H-1,2,4-triazol-3-amine (0.05 g, 0.22 mmol) and 5-Bromopyrazine-2-carbonitrile (0.04 g, 0.20 mmol) were reacted as per General Procedure 4 Method 1, using 5 mol % Pd₂(dba)₃, 10 mol % Xantphos, 1.5 eq Cs₂CO₃ and heated at 90° C. for 16 h. The crude residue was purified as per General Procedure 4 Method 1 to give 5-((1-(5-(trifluoromethyl)pyridin-2-yl)-1H-1,2,4-triazol-3-yl)amino)pyrazine-2-carbonitrile (0.02 g, 25% yield) as a grey solid. ¹H NMR (401 MHz, DMSO-d₆) δ 11.13 (s, 1H), 9.44 (s, 1H), 9.27 (s, 1H), 8.96 (s, 1H), 8.83 (s, 1H), 8.49 (dd, J=8.6, 2.3 Hz, 1H), 8.07 (d, J=8.6 Hz, 1H); LCMS R_{f}(min)=2.88. MS m/z=333.1 [M+H]⁺.

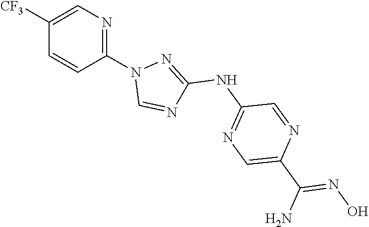 [SMILES: N/C(=N\O)c1cnc(Nc2ncn(-c3ccc(C(F)(F)F)cn3)n2)cn1]

5-((1-(5-(Trifluoromethyl)pyridin-2-yl)-1H-1,2,4-triazol-3-yl)amino)pyrazine-2-carbonitrile (0.02 g, 0.06 mmol) was reacted as per General Procedure 1 Method 3 using NH₂OH·HCl (0.02 g, 0.22 mmol) and NEt₃ (0.03 mL, 0.22 mmol) in dry MeOH (0.3 mL) to give N′-hydroxy-5-((1-(5-(trifluoromethyl)pyridin-2-yl)-1H-1,2,4-triazol-3-yl)amino)pyrazine-2-carboximidamide (0.02 g, 74%) as white solid. ¹H NMR (401 MHz, DMSO-d₆) δ 10.81 (s, 1H), 9.89 (s, 1H), 9.38 (s, 1H), 9.26 (s, 1H), 8.95 (s, 1H), 8.72 (d, J=0.7 Hz, 1H), 8.49 (dd, J=8.6, 0.9 Hz, 1H), 8.04 (d, J=8.3 Hz, 1H), 5.82 (s, 2H); LCMS R_{f}(min)=2.51, MS m/z=366.1 [M+H]⁺. HRMS (ESI) calcd for C₁₃H₁₁F₃N₉O [M+H]⁺ 366.1033, found 366.1021.

### 109. N′-Hydroxy-5-((1-methyl-5-(5-(trifluoromethyl)pyridin-2-yl)-1H-pyrazol-3-yl)amino)picolinimidamide (Scheme 106)

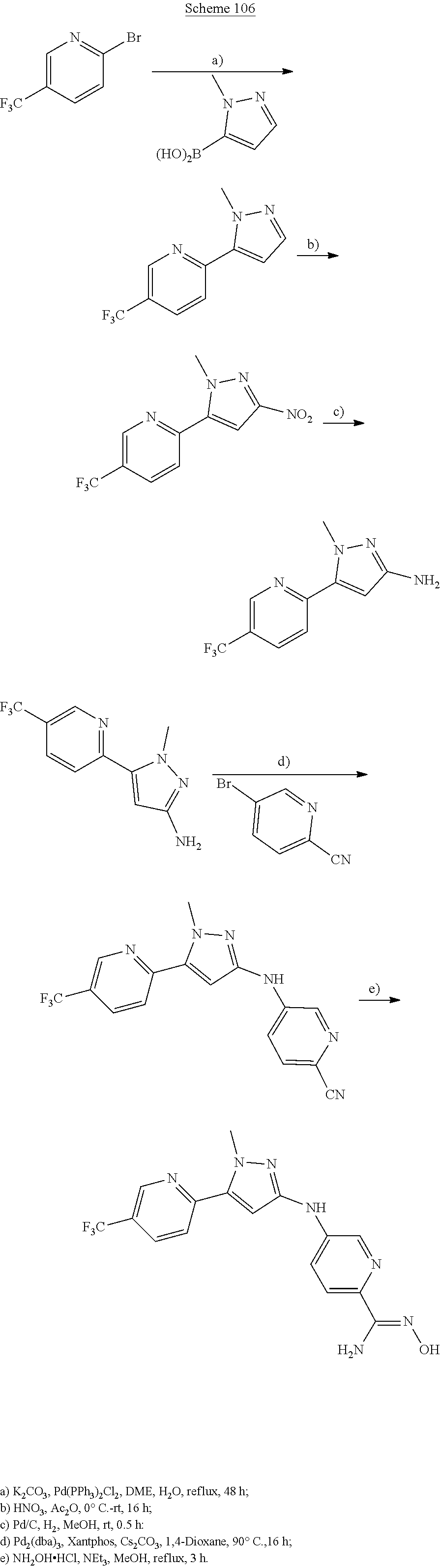

A suspension of 2-bromo-5-(trifluoromethyl)pyridine (5.38 g, 23.83 mmol), (1-methyl-1H-pyrazol-5-yl)boronic acid (3.0 g, 23.83 mmol), K₂CO₃ (13.17 g, 95.30 mmol), Pd(PPh₃)₂Cl₂ (0.84 g, 1.19 mmol) in DME (95 mL) and H₂O (10 mL) was heated to reflux for 48 h. The volatiles were removed in vacuo then H₂O (100 mL) was added, extracted with EtOAc (2×100 mL) and washed with brine (100 mL). The combined organic layer was dried over MgSO₄ and concentrated under reduced pressure. The resultant crude residue was purified by flash chromatography, eluting with 20-30% EtOAc in petroleum spirits to give 2-(1-methyl-1H-pyrazol-5-yl)-5-(trifluoromethyl)pyridine (1.85 g, 34% yield) as a yellow solid. ¹H NMR (401 MHz, DMSO-d₆) δ 9.06 (dd, J=1.5, 0.9 Hz, 1H), 8.31 (ddd, J=8.4, 2.4, 0.6 Hz, 1H), 8.04 (d, J=8.4 Hz, 1H), 7.55 (d, J=2.0 Hz, 1H), 6.98 (d, J=2.0 Hz, 1H), 4.18 (s, 3H). LCMS R_{f}(min)=2.88, MS m/z=228.1 [M+H]⁺.

HNO₃ (37% aq., 1.7 mL) was slowly added into a solution of 2-(1-methyl-1H-pyrazol-5-yl)-5-(trifluoromethyl)pyridine in Ac₂O (10 mL) at 0° C. The reaction mixture was slowly warmed up to rt and allowed to stir for 16 h. The reaction mixture was poured into ice water. To this cold aqueous mixture was added sat. aq. NaHCO₃ (aq.) (25 mL), followed by extraction with EtOAc (2×25 mL). The combined organic layer was washed with H₂O (25 mL) and brine (25 mL), then dried over MgSO₄ and concentrated under reduced pressure. The resultant crude residue was purified by flash chromatography, eluting with 15-20% EtOAc in petroleum spirits to give 2-(1-methyl-3-nitro-1H-pyrazol-5-yl)-5-(trifluoromethyl)pyridine (0.67 g, 59% yield) as a white solid. ¹H NMR (401 MHz, DMSO-d₆) δ 9.14 (dd, J=1.4, 0.8 Hz, 1H), 8.44 (dd, J=8.4, 1.9 Hz, 1H), 8.24 (d, J=8.4 Hz, 1H), 7.87 (s, 1H), 4.30 (s, 3H). LCMS R_{f}(min)=3.01, MS m/z=273.0 [M+H]⁺.

A solution of 2-(1-methyl-3-nitro-1H-pyrazol-5-yl)-5-(trifluoromethyl)pyridine (0.67 g, 2.46 mmol) in MeOH (25 mL) was evacuated and backfilled with N₂×3, then 10% Pd/C (0.33 g) was added. The resultant suspension was evacuated and flushed with H₂×3 and stirred under a hydrogen atmosphere (balloon) for 0.5 h. The reaction mixture was filtered through a pad of celite and the filtrate was concentrated under reduced pressure to give 1-methyl-5-(5-(trifluoromethyl)pyridin-2-yl)-1H-pyrazol-3-amine (0.50 g, 83% yield) as an off-white solid. ¹H NMR (401 MHz, DMSO-d₆) δ 9.00 (s, 1H), 8.23 (dd, J=8.4, 2.3 Hz, 1H), 7.89 (d, J=8.4 Hz, 1H), 6.05 (s, 1H), 4.74 (s, 2H), 3.93 (s, 3H). LCMS R_{f} (min)=3.22, MS m/z=243.1 [M+H]⁺.

1-Methyl-5-(5-(trifluoromethyl)pyridin-2-yl)-1H-pyrazol-3-amine (0.1 g, 0.41 mmol) and 5-bromopicolinonitrile (0.069 g, 0.38 mmol) were reacted as per General Procedure 4 Method 1, using 5 mol % Pd₂(dba)₃, 10 mol % Xantphos, 1.5 eq Cs₂CO₃ and heated at 90° C. for 16 h. The crude residue was purified by flash chromatography, eluting with 50-70% EtOAc in petroleum spirits to give 5-((1-methyl-5-(5-(trifluoromethyl)pyridin-2-yl)-1H-pyrazol-3-yl)amino)picolinonitrile (0.09 g, 70% yield) as a yellow solid. ¹H NMR (401 MHz, DMSO-d₆) δ 9.71 (s, 1H), 9.09 (s, 1H), 8.62 (d, J=2.5 Hz, 1H), 8.34 (dd, J=8.6, 2.2 Hz, 1H), 8.09 (d, J=8.2 Hz, 1H), 8.01 (dd, J=8.7, 2.7 Hz, 1H), 7.83 (d, J=8.5 Hz, 1H), 6.64 (s, 1H), 4.13 (s, 3H); LCMS R_{f} (min)=2.89, MS m/z=345.1 [M+H]⁺.

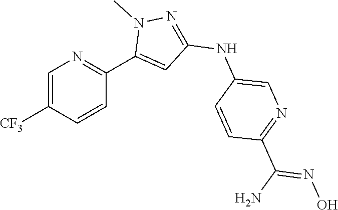 [SMILES: Cn1nc(Nc2ccc(/C(N)=N/O)nc2)cc1-c1ccc(C(F)(F)F)cn1]

5-((1-Methyl-5-(5-(trifluoromethyl)pyridin-2-yl)-1H-pyrazol-3-yl)amino)picolinonitrile (0.09 g, 0.26 mmol) was reacted as per General Procedure 1 Method 3 using NH₂OH·HCl (0.07 g, 1.06 mmol) and NEt₃ (0.1 mL, 0.70 mmol) in dry MeOH (1.3 mL) to give N′-hydroxy-5-((1-methyl-5-(5-(trifluoromethyl)pyridin-2-yl)-1H-pyrazol-3-yl)amino)picolinimidamide (0.09 g, 93% yield) as a yellow solid. ¹H NMR (401 MHz, DMSO-d₆) δ 9.58 (s, 1H), 9.07 (m, 2H), 8.59 (d, J=2.3 Hz, 1H), 8.32 (d, J=8.3 Hz, 1H), 8.07 (d, J=8.4 Hz, 1H), 7.84 (dd, J=8.8, 2.5 Hz, 1H), 7.71 (d, J=8.8 Hz, 1H), 6.55 (s, 1H), 5.68 (s, 2H), 4.11 (s, 3H); ¹³C NMR (101 MHz, DMSO-d₆) δ 152.4, 149.7, 149.3, 140.2, 140.0, 139.9, 135.2, 134.8, 125.1, 123.5, 123.0, 122.4, 121.5, 119.6, 96.1, 39.2. LCMS R_{f}(min)=2.42, MS m/z=378.1 [M+H]⁺. HRMS (ESI) calcd for C₁₆H₁₅F₃N₇O [M+H]⁺ 378.1285, found 378.1294.

### 110. N′-Hydroxy-5-((1-methyl-5-(5-(trifluoromethyl)pyridin-2-yl)-1H-pyrazol-3-yl)amino)pyrazine-2-carboximidamide (Scheme 107)

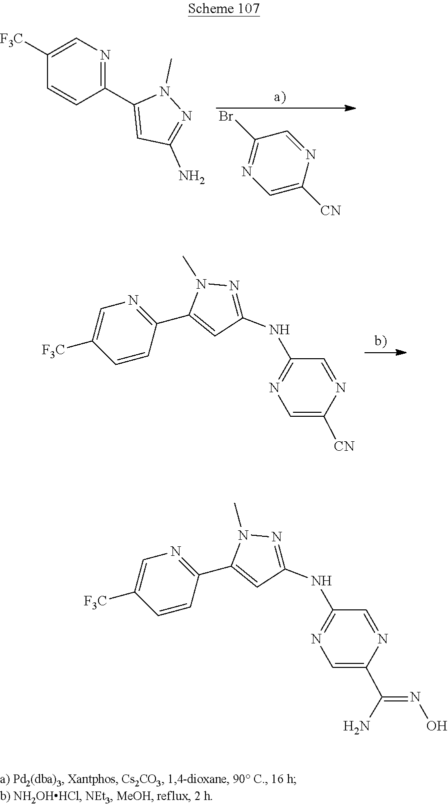

1-Methyl-5-(5-(trifluoromethyl)pyridin-2-yl)-1H-pyrazol-3-amine (0.1 g, 0.41 mmol) and 5-bromopyrazine-2-carbonitrile (0.069 g, 0.38 mmol) were reacted as per General Procedure 4 Method 1, using 5 mol % Pd₂(dba)₃, 10 mol % Xantphos, 1.5 eq Cs₂CO₃ and heated at 90° C. for 16 h. The reaction mixture was diluted with EtOAc (10 mL), washed with H₂O (10 mL) and brine (10 mL). The combined organic layer was dried over MgSO₄ and concentrated under reduced pressure. The crude residue was purified by flash chromatography, eluting with 25-30% EtOAc in petroleum spirit to give 5-((1-methyl-5-(5-(trifluoromethyl)pyridin-2-yl)-1H-pyrazol-3-yl)amino)pyrazine-2-carbonitrile (0.04 g, 34% yield) as a pale-yellow solid. ¹H NMR (401 MHz, DMSO-d₆) δ 10.93 (s, 1H), 9.09 (s, 1H), 8.68 (d, J=1.3 Hz, 1H), 8.44 (s, 1H), 8.32 (d, J=8.1 Hz, 1H), 8.05 (d, J=8.2 Hz, 1H), 7.22 (s, 1H), 4.14 (s, 3H); LCMS R_{f}(min)=2.98, MS m/z=346.0 [M+H]⁺.

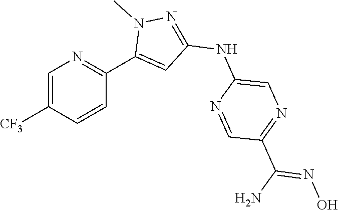 [SMILES: Cn1nc(Nc2cnc(/C(N)=N/O)cn2)cc1-c1ccc(C(F)(F)F)cn1]

5-((1-Methyl-5-(5-(trifluoromethyl)pyridin-2-yl)-1H-pyrazol-3-yl)amino)pyrazine-2-carbonitrile (0.03 g, 0.08 mmol) was reacted as per General Procedure 1 Method 3 using NH₂OH·HCl (0.02 g, 0.3 mmol) and NEt₃ (0.04 mL, 0.3 mmol) in dry MeOH (0.5 mL) to give N′-hydroxy-5-((1-methyl-5-(5-(trifluoromethyl)pyridin-2-yl)-1H-pyrazol-3-yl)amino)pyrazine-2-carboximidamide (0.02 g, 76% yield) as a white solid. ¹H NMR (401 MHz, DMSO-d₆) δ 10.11 (s, 1H), 9.72 (s, 1H), 9.07 (dd, J=1.3, 0.8 Hz, 1H), 8.60 (d, J=1.4 Hz, 1H), 8.47 (d, J=1.4 Hz, 1H), 8.30 (dd, J=8.5, 2.0 Hz, 1H), 8.05 (d, J=8.4 Hz, 1H), 7.11 (s, 1H), 5.73 (s, 2H), 4.12 (s, 3H); ¹³C NMR (101 MHz, DMSO-d₆) δ 152.4, 151.1, 148.7, 147.2, 139.6, 138.6, 135.4, 131.1, 122.8, 97.8, 39.3; LCMS R_{f} (min)=2.39, MS m/z=379.1 [M+H]⁺. HRMS (ESI) calcd for C₁₅H₁₄F₃N₈O [M+H]⁺ 379.1237, found 379.1253.

### 111. N′-Hydroxy-5-((1-(4-(trifluoromethyl)phenyl)-1H-pyrazol-3-yl)amino)pyrazine-2-carboximidamide (Scheme 108)

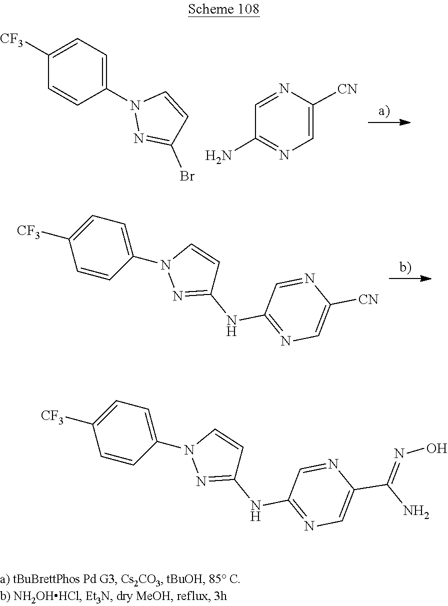

3-Bromo-1-(4-(trifluoromethyl)phenyl)-1H-pyrazole (Intermediate O, 1 eq.) and 5-aminopyrazine-2-carbonitrile (1.4 eq) were reacted as per General Procedure 4 Method 2 using 5 mol % Pd precatalyst, 4 eq. Cs₂CO₃ and heated at 85° C. for 20 h. The crude product was purified using 25-35% EtOAc in petroleum spirits gradient to give 5-((1-(4-(trifluoromethyl)phenyl)-1H-pyrazol-3-yl)amino)pyrazine-2-carbonitrile (98.5 mg, 84% yield). ¹H NMR (401 MHz, DMSO-d₆) δ 8.72 (d, J=1.3 Hz, 1H), 8.66 (d, J=2.7 Hz, 1H), 8.62 (s, 1H), 8.04 (d, J=8.5 Hz, 2H), 7.87 (d, J=8.6 Hz, 2H), 6.91 (d, J=2.0 Hz, 1H). LCMS R_{f}(min)=3.96, MS m/z 331.1 [M+H]⁺.

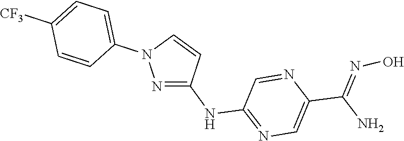 [SMILES: N/C(=N\O)c1cnc(Nc2ccn(-c3ccc(C(F)(F)F)cc3)n2)cn1]

5-((1-(4-(Trifluoromethyl)phenyl)-1H-pyrazol-3-yl)amino)pyrazine-2-carbonitrile was reacted as per General Procedure 1 Method 3 using 4 eq. NH₂OH·HCl and 4 eq. Et₃N for 3 h to give N′-hydroxy-5-((1-(4-(trifluoromethyl)phenyl)-1H-pyrazol-3-yl)amino)pyrazine-2-carboximidamide as a white solid (84.2 mg, 83%) ¹H NMR (401 MHz, DMSO-d₆) δ 10.34 (s, 1H), 9.79 (s, 1H), 8.77 (d, J=1.5 Hz, 1H), 8.64 (d, J=1.5 Hz, 1H), 8.61 (d, J=2.7 Hz, 1H), 8.02 (d, J=8.5 Hz, 2H), 7.86 (d, J=8.6 Hz, 2H), 6.74 (d, J=2.7 Hz, 1H), 5.78 (s, 2H). ¹³C NMR (101 MHz, DMSO-d₆) δ 151.0, 150.7, 148.7, 142.3, 138.9, 136.3, 131.1, 128.9, 126.9, 126.9, 125.6, 125.3, 125.0, 122.9, 117.3, 100.2. LCMS R_{f} (min)=2.52, MS m/z 364.1 [M+H]⁺. HRMS (ESI) calcd for C₁₅H₁₃F₃N₇O⁺ [M+H]⁺ 364.1128, found 364.1133.

### 112. N′-Hydroxy-5-((1-(5-(trifluoromethyl)pyridin-2-yl)-1H-pyrazol-3-yl)amino)pyrazine-2-carboximidamide (Scheme 109)

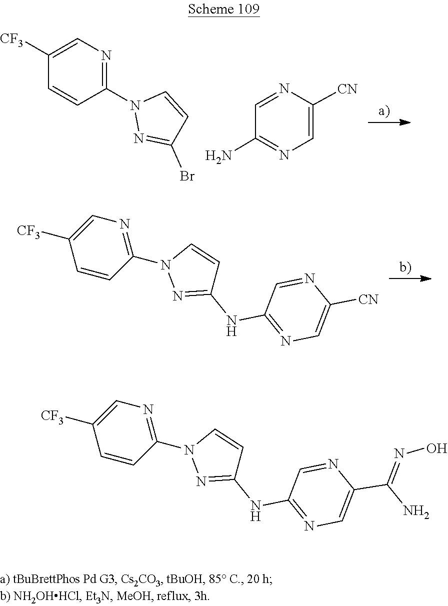

2-(3-Bromo-1H-pyrazol-1-yl)-5-(trifluoromethyl)pyridine (Intermediate N, 1 eq.) and 5-aminopyrazine-2-carbonitrile (1.4 eq) were reacted as per General Procedure 4 Method 2 using 6 mol % Pd precatalyst, 3.8 eq. Cs₂CO₃ and heated at 85° C. for 20 h. The crude product was purified using 25-35% EtOAc in petroleum spirits gradient to give 5-((1-(5-(trifluoromethyl)pyridin-2-yl)-1H-pyrazol-3-yl)amino)pyrazine-2-carbonitrile (97.3 mg, 82% yield). LCMS R_{f}(min)=4.00, MS m/z 332.1 [M+H]⁺.

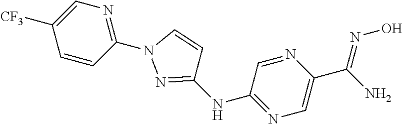 [SMILES: N/C(=N\O)c1cnc(Nc2ccn(-c3ccc(C(F)(F)F)cn3)n2)cn1]

5-((1-(5-(Trifluoromethyl)pyridin-2-yl)-1H-pyrazol-3-yl)amino)pyrazine-2-carbonitrile was reacted as per General Procedure 1 Method 3 using 4 eq. NH₂OH·HCl and 4 eq. Et₃N for 3 h to give N′-hydroxy-5-((1-(5-(trifluoromethyl)pyridin-2-yl)-1H-pyrazol-3-yl)amino)pyrazine-2-carboximidamide as a white solid (63.8 mg, 67%) ¹H NMR (401 MHz, DMSO-d₆) δ 10.45 (s, 1H), 9.83 (s, 1H), 8.87-8.84 (m, 2H), 8.67 (d, J=1.5 Hz, 1H), 8.62 (d, J=2.8 Hz, 1H), 8.39 (dd, J=9.0, 2.2 Hz, 1H), 7.97 (d, J=8.7 Hz, 1H), 6.78 (d, J=2.8 Hz, 1H), 5.80 (s, 2H). ¹³C NMR (101 MHz, DMSO-d₆) δ 152.9, 152.1, 150.4, 148.6, 145.8, 138.9, 137.1, 137.1, 136.7, 131.2, 128.5, 125.2, 122.5, 121.9, 121.6, 111.1, 101.2. LCMS R_{f}(min)=2.52, MS m/z 365.1 [M+H]⁺. HRMS (ESI) calcd for C₁₄H₁₂F₃N₈O⁺ [M+H]⁺ 365.1081, found 365.1085.

### 113. N′-Hydroxy-5-((1-(4-(trifluoromethyl)phenyl)-1H-pyrazol-4-yl)amino)picolinimidamide (Scheme 110)

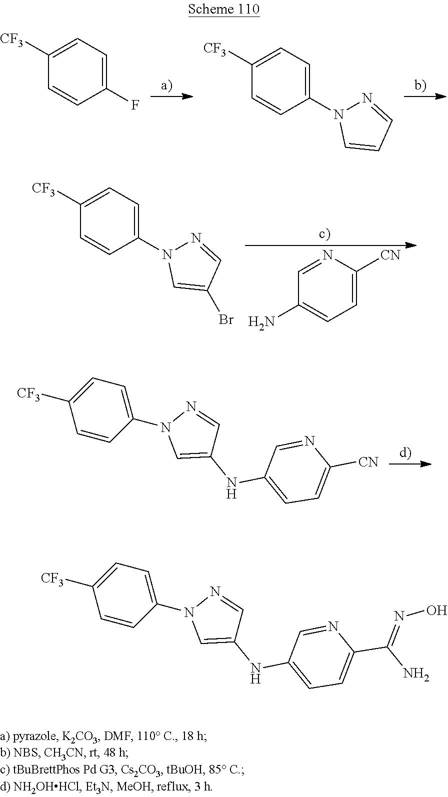

1-Fluoro-4-(trifluoromethyl)benzene (5 g, 30.46 mmol) was stirred with pyrazole (2.5 g, 36.6 mmol) in DMF (60 mL) at 110° C. for 18 h. The reaction mixture was cooled to room temperature, diluted with Et₂O and water. After separation the aqueous phase was extracted with Et₂O (4×), the combined organic phases were dried over (MgSO₄), evaporated to dryness. The residue was purified on silica gel using mixtures of petroleum spirits and DCM as eluents to provide 1-(4-(trifluoromethyl)phenyl)-1H-pyrazole in 37.1% yield (2.4 g). ¹H NMR (401 MHz, CDCl₃) δ 7.98 (dd, J=2.6, 0.4 Hz, 1H), 7.82 (d, J=8.4 Hz, 2H), 7.75 (d, J=1.6 Hz, 1H), 7.70 (d, J=8.5 Hz, 2H), 6.51 (dd, J=2.5, 1.8 Hz, 1H). LCMS R_{f}(min)=3.062, MS m/z=213.0 [M+H]⁺.

1-(4-(Trifluoromethyl)phenyl)-1H-pyrazole (2.4 g, 11.31 mmol) was stirred with NBS (3 g, 17 mmol) in CH₃CN (16 mL) at rt for 48 h. The volatile solvents were removed and the residue taken up in DCM and saturated NaHCO₃ solutions. After separation the aqueous phase was extracted with DCM (4×), the combined organic phases were dried over (MgSO₄), evaporated to dryness. The residue was purified on silica gel using mixtures of petroleum spirits and DCM as eluents to provide 4-bromo-1-(4-(trifluoromethyl)phenyl)-1H-pyrazole in 36.5% yield (1.2 g). ¹H NMR (401 MHz, CDCl₃) δ 8.00 (s, 1H), 7.78 (d, J=8.7 Hz, 2H), 7.73 (m, 3H). LCMS R_{f}(min)=4.197, MS m/z=291.0/293.0 [M+H]⁺. 4-Bromo-1-(4-(trifluoromethyl)phenyl)-1H-pyrazole (1 eq.) and 5-aminonicotinonitrile (1.4 eq) were reacted as per General Procedure 4 Method 2 using 5 mol % Pd precatalyst, 2.8 eq. Cs₂CO₃ and heated at 85° C. for 20 h. The crude product was purified using 10-100% EtOAc in petroleum spirits gradient to give 5-((1-(4-(trifluoromethyl)phenyl)-1H-pyrazol-4-yl)amino)picolinonitrile (58.5 mg, 50% yield). ¹H NMR (401 MHz, DMSO-d₆) δ 9.16 (s, 1H), 8.78 (s, 1H), 8.33 (d, J=2.6 Hz, 1H), 8.11 (d, J=8.5 Hz, 2H), 7.90-7.85 (m, 3H), 7.78 (d, J=8.7 Hz, 1H), 7.39 (dd, J=8.7, 2.9 Hz, 1H). LCMS R_{f}(min)=3.87, MS m/z 330.1 [M+H]⁺.

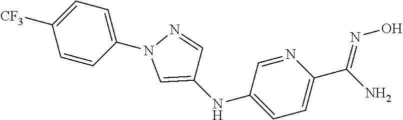 [SMILES: N/C(=N\O)c1ccc(Nc2cnn(-c3ccc(C(F)(F)F)cc3)c2)cn1]

5-((1-(4-(Trifluoromethyl)phenyl)-1H-pyrazol-4-yl)amino)picolinonitrile was reacted as per General Procedure 1 Method 3 using 4 eq. NH₂OH·HCl and 4 eq. Et₃N for 3 h to give N′-hydroxy-5-((1-(4-(trifluoromethyl)phenyl)-1H-pyrazol-4-yl)amino)picolinimidamide as a white solid (34.9 mg, 54%) ¹H NMR (401 MHz, DMSO-d₆) δ 9.59 (s, 1H), 8.68 (s, 1H), 8.53 (s, 1H), 8.24 (d, J=2.3 Hz, 1H), 8.11 (d, J=8.5 Hz, 2H), 7.86 (d, J=8.6 Hz, 2H), 7.79 (s, 1H), 7.70 (d, J=8.8 Hz, 1H), 7.38 (dd, J=8.8, 2.8 Hz, 1H), 5.67 (s, 2H). ¹³C NMR (101 MHz, DMSO-d₆) δ 149.6, 142.5, 141.7, 139.6, 136.0, 134.2, 127.3, 126.8, 126.8, 125.8, 125.6, 125.5, 125.2, 122.9, 120.0, 120.0 117.9, 117.1. LCMS R_{f}(min)=3.28, MS m/z 363.1 [M+H]⁺. HRMS (ESI) calcd for C₁₆H₁₄F₃N₆O⁺ [M+H]⁺ 363.1176, found 363.1187.

### 114. N′-Hydroxy-5-((1-(4-(trifluoromethyl)phenyl)-1H-pyrazol-4-yl)amino)pyrazine-2-carboximidamide (Scheme 111)

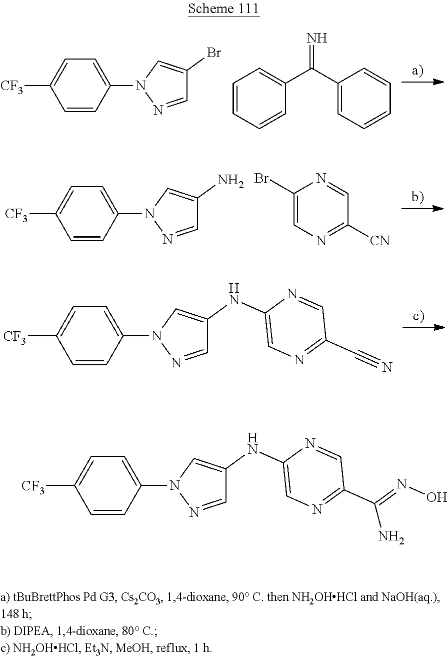

4-Bromo-1-(4-(trifluoromethyl)phenyl)-1H-pyrazole (0.314 g, 1.08 mmol), Cs₂CO₃ (1.01 g, 3.1 mmol) and tBuBrettPhos Pd G3 precatalyst (0.044 g, 0.052 mmol) and diphenylmethanimine (0.35 ml, 2.1 mmol) were placed in round bottom flask under N₂. Anhydrous dioxane (4 mL) was added and the reaction was heated to 90° C. for 20 h, cooled then NH₂OH·HCl (0.285 g, 4.1 mmol) and 2 M aq. NaOH were added (2 mL, 4 mmol) and the mixture was stirred at room temperature. After 24 h, the mixture was diluted with 4 mL dioxane, 4 mL MeOH and 2 mL water and NH₂OH·HCl (0.285 g, 4.1 mmol) was added. After stirring for 24 h the mixture was added to 400 mL water and the resulting solid was collected by filtration, dried, dissolved in EtOAc and concentrated onto silica. The crude product was purified by flash chromatography (25-100% EtOAc in DCM) to give 1-(4-(trifluoromethyl)phenyl)-1H-pyrazol-4-amine (0.0525 g, 0.231 mmol, 21%) ¹H NMR (401 MHz, CDCl₃) δ 7.87-7.86 (m, 1H), 7.77 (d, J=8.9 Hz, 2H), 7.72 (d, J=8.8 Hz, 2H), 7.64 (s, 1H). LCMS R_{f}(min)=3.13, MS m/z 228.1 [M+H]⁺. 1-(4-(Trifluoromethyl)phenyl)-1H-pyrazol-4-amine (0.0525 g, 0.231 mmol) and 5-bromopyrazine-2-carbonitrile (0.127 g, 0.693 mmol) were placed under N₂ in a screw cap vial, anhydrous dioxane (2 mL) and diisopropylethylamine (0.12 mL, 0.69 mmol) were added and the vial was capped then heated to 80° C. for 22 h. The reaction was cooled, diluted with EtOAc, concentrated onto silica and purified by flash chromatography (25-50% EtOAc in petroleum spirits) to give 5-((1-(4-(trifluoromethyl)phenyl)-1H-pyrazol-4-yl)amino)pyrazine-2-carbonitrile (0.0525 g, 0.159 mmol, 69%) ¹H NMR (401 MHz, DMSO-d₆) δ 8.84 (s, 1H), 8.66 (s, 1H), 8.23 (s, 1H), 8.06 (d, J=8.5 Hz, 2H), 8.01 (s, 1H), 7.87 (d, J=8.7 Hz, 2H). LCMS R_{f}(min)=3.89, MS m/z 331.1 [M+H]⁺.

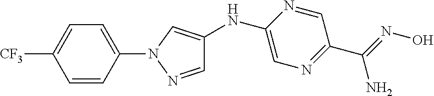 [SMILES: N/C(=N\O)c1cnc(Nc2cnn(-c3ccc(C(F)(F)F)cc3)c2)cn1]

5-((1-(4-(Trifluoromethyl)phenyl)-1H-pyrazol-4-yl)amino)pyrazine-2-carbonitrile was reacted as per General Procedure 1 Method 3 using 4 eq. NH₂OH·HCl and 4 eq. Et₃N for 1 h with addition of water to induce precipitation and washing with DCM prior to drying to give N′-hydroxy-5-((1-(4-(trifluoromethyl)phenyl)-1H-pyrazol-4-yl)amino)pyrazine-2-carboximidamide as a cream solid (36.5 mg, 0.100 mmol, 63%) ¹H NMR (401 MHz, DMSO-d₆) δ 9.86 (s, 1H), 9.71 (s, 1H), 8.82 (s, 1H), 8.64 (d, J=1.4 Hz, 1H), 8.16 (d, J=1.4 Hz, 1H), 8.06 (d, J=8.5 Hz, 2H), 7.93 (s, 1H), 7.85 (d, J=8.6 Hz, 2H), 5.72 (s, 2H). LCMS R_{f} (min)=3.25. MS m/z 364.1 [M+H]⁺. HRMS (ESI) calcd for C₁₅H₁₂F₃N₆O₂⁺ [M+H]⁺ 364.1128, found 364.1133.

### 115. N′-Hydroxy-5-((1-(5-(trifluoromethyl)pyridin-2-yl)-1H-1,2,3-triazol-4-yl)amino)picolinimidamide (Scheme 112)

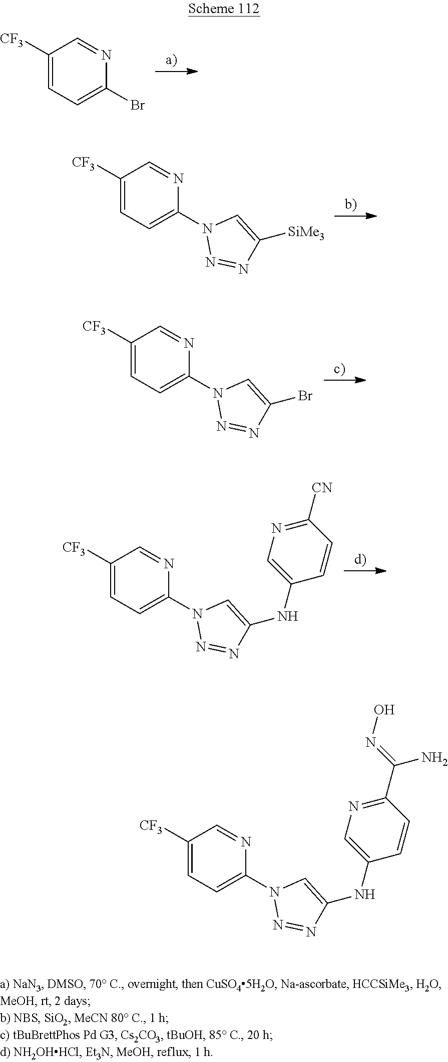 [SMILES: FC(F)(F)c1ccc(Br)nc1 ; C[Si](C)(C)c1cn(-c2ccc(C(F)(F)F)cn2)nn1 ; FC(F)(F)c1ccc(-n2cc(Br)nn2)nc1 ; N#Cc1ccc(Nc2cn(-c3ccc(C(F)(F)F)cn3)nn2)cn1 ; N/C(=N\O)c1ccc(Nc2cn(-c3ccc(C(F)(F)F)cn3)nn2)cn1]

To a solution of 2-bromo-5-(trifluoromethyl)pyridine (1.5 g, 6.64 mmol) in DMSO (33 ml) was added sodium azide (0.86 g, 13.3 mmol) at room temperature, and the mixture stirred at 70° C. overnight. The reaction solution was diluted with Et₂O, and the mixture was washed with water and brine. The organic layer was dried over anhydrous MgSO₄ to give an Et₂O solution of 2-azido-5-(trifluoromethyl)pyridine which then used directly in the following step after analysis by LCMS. LCMS R_{f} (min)=3.15, MS m/z 189.1 [M+H]+, 163.0 [M-N₂+3H]⁺. A separate 500 mL round-bottom flask was charged with CuSO₄·5H₂O (0.83 g, 3.30 mmol) and Na-ascorbate (1.31 g, 6.63 mmol). With stirring, H₂O (70 mL) and MeOH (70 mL) were added. To this mixture was added ethynyltrimethylsilane (1.30 g, 13.3 mmol) followed by the above prepared solution of 2-azido-5-(trifluoromethyl)pyridine (6.64 mmol) in Et₂O. After stirring the reaction mixture at ambient temperature overnight, CuSO₄·5H₂O (0.83 g, 3.30 mmol) and Na-ascorbate (1.31 g, 6.63 mmol) were added, and the stirring continued overnight. The volatile solvents were removed in vacuo, and the residue stirred with a mixtures of 15% aq. NH₄OH and DCM (100 mL each). The mixture was filtered through a pad of celite, which was washed with DCM (3×). These washing DCM solutions were used to extract the aqueous phase. The combined organic solvents were dried over MgSO₄, filtered and evaporated to dryness. The residue was dissolved in EtOAc, concentrated onto silica gel and purified by flash chromatography (10-35% EtOAc in petroleum spirits) to give 5-(trifluoromethyl)-2-(4-(trimethylsilyl)-1H-1,2,3-triazol-1-yl)pyridine (10.6%, 0.201 g, 0.701 mmol). ¹H NMR (401 MHz, CDCl₃) δ 8.78-8.76 (m, 1H), 8.59 (s, 1H), 8.38-8.34 (m, 1H), 8.17-8.13 (m, 1H), 0.39 (s, 9H). LCMS R_{f}(min)=4.24, MS m/z 287.1 [M+H]⁺

To a solution of 5-(trifluoromethyl)-2-(4-(trimethylsilyl)-1H-1,2,3-triazol-1-yl)pyridine (0.131 g, 0.46 mmol) in MeCN (3 mL) was added silica gel (0.712 g, 11.9 mmol) and N-bromosuccinimide (0.285 g, 1.6 mmol), the reaction was heated to 80° C. for 1 h, cooled, filtered, the silica gel was washed with EtOAc. The filtrate was washed with mixture of water and brine, then 5% aq. Na₂S₂O₃ then brine, dried with MgSO₄, filtered and concentrated. The residue was dissolved in minimal petroleum spirits and DCM and purified by silica gel flash chromatography (5-10% EtOAc in petroleum spirits) to give 2-(4-bromo-1H-1,2,3-triazol-1-yl)-5-(trifluoromethyl)pyridine (0.0360 g, 0.123 mmol, 27%). ¹H NMR (401 MHz, CDCl₃) δ 8.80-8.77 (m, 1H), 8.63 (s, 1H), 8.36-8.32 (m, 1H), 8.21-8.17 (m, 1H).

2-(4-Bromo-1H-1,2,3-triazol-1-yl)-5-(trifluoromethyl)pyridine (1 eq.) and 5-aminonicotinonitrile (1.5 eq.) were reacted as per General Procedure 4 Method 2 using 10 mol % Pd precatalyst and 3 eq. Cs₂CO₃ and heated at 85° C. for 20 h with the following alteration to the work up, the reaction was diluted with EtOAc and water, the phases were separated and the aq. layer extracted twice with EtOAc, the combined organic layers were washed sequentially with 5% aq. citric acid, water and brine, dried with MgSO₄, filtered and concentrated. The residue was dissolved in MeOH, EtOAc and 0.1 mL Et₃N then concentrated onto silica gel and purified using silica gel flash chromatography (25-100% EtOAc in petroleum spirits). Fractions containing product were combined, washed with water then brine, dried with MgSO₄, filtered, concentrated and triturated with warm MeOH to remove residual 5-aminonicotinonitrile to give 5-((1-(5-(trifluoromethyl)pyridin-2-yl)-1H-1,2,3-triazol-4-yl)amino)picolinonitrile (0.0122 g, 0.0368 mmol, 50%) ¹H NMR (401 MHz, DMSO-d₆) δ 9.97 (s, 1H), 9.05 (s, 1H), 8.71 (s, 1H), 8.57-8.53 (m, 2H), 8.36 (d, J=8.6 Hz, 1H), 7.87 (d, J=8.6 Hz, 1H), 7.80 (dd, J=8.7, 2.6 Hz, 1H). LCMS R_{f} (min)=3.66, MS m/z 332.1 [M+H]⁺.

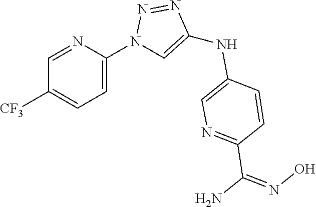 [SMILES: N/C(=N/O)c1ccc(Nc2cn(-c3ccc(C(F)(F)F)cn3)nn2)cn1]

5-((1-(5-(Trifluoromethyl)pyridin-2-yl)-1H-1,2,3-triazol-4-yl)amino)picolinonitrile was reacted as per General procedure 1 Method 3 using 4 eq. NH₂OH·HCl and 4 eq. Et₃N to give N′-hydroxy-5-((1-(5-(trifluoromethyl)pyridin-2-yl)-1H-1,2,3-triazol-4-yl)amino)picolinimidamide as a white solid (0.002 g, 15%) ¹H NMR (401 MHz, DMSO-d₆) δ 9.63 (s, 1H), 9.39 (s, 1H), 9.05-9.03 (m, 1H), 8.56-8.51 (m, 2H), 8.49-8.46 (m, 1H), 8.37-8.33 (m, 1H), 7.76 (d, J=8.8 Hz, 1H), 7.69 (dd, J=8.8, 2.7 Hz, 1H), 5.70 (s, 2H). LCMS R_{f}(min)=3.14, MS m/z 365.2 [M+H]⁺. HRMS (ESI) calcd for C₁₄H₁₂F₃N₈O [M+H]⁺ 365.1081, found 365.1088.

### 116. rac-5-((5-(5-Chloropyridin-2-yl)oxazol-2-yl)amino)-N-(2,3-dihydroxypropyl)picolinamide hydrochloride (Scheme 113)

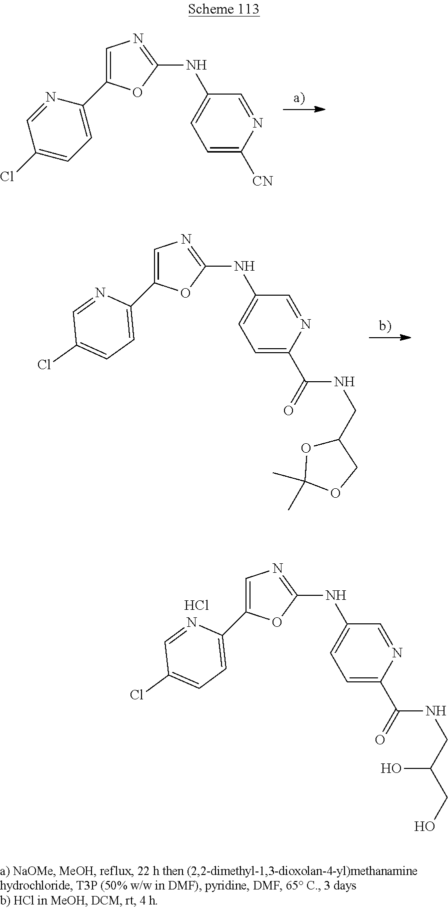 [SMILES: N#Cc1ccc(Nc2ncc(-c3ccc(Cl)cn3)o2)cn1 ; CC1(C)OCC(CNC(=O)c2ccc(Nc3ncc(-c4ccc(Cl)cn4)o3)cn2)O1 ; O=C(NCC(O)CO)c1ccc(Nc2ncc(-c3ccc(Cl)cn3)o2)cn1 ; Cl]

5-((5-(5-Chloropyridin-2-yl)oxazol-2-yl)amino)picolinonitrile (0.3084 g, 1.04 mmol) was added to 4 mL of a 1.9 M solution of NaOMe (7.6 mmol) (prepared from addition of Na metal to anhydrous MeOH) and heated in a bath set to 110° C. for 22 h under a N₂ atmosphere. After this time the solvent had evaporated. The cooled residue was dissolved in a mixture of 2 M NaOH (5 mL) and water (200 mL) and then washed with EtOAc (2×50 mL), the aq. layer was acidified with 0.5 M aq. HCl to pH=4 giving a precipitate which was collected by filtration and washed with Et₂O, giving 119 mg of a solid which was determined by LCMS to consist of a mixture of 5-((5-(5-chloropyridin-2-yl)oxazol-2-yl)amino)picolinic acid and 5-((5-(5-chloropyridin-2-yl)oxazol-2-yl)amino)picolinamide in an approximately 2:1 ratio in favour of the carboxylic acid. LCMS of 5-((5-(5-chloropyridin-2-yl)oxazol-2-yl)amino)picolinic acid: R_{f} (min)=3.15, MS m/z 317.1 [M+H]⁺. This mixture was used without further purification, and was dissolved in 3 mL anhydrous DMF, rac-(2,2-dimethyl-1,3-dioxolan-4-yl)methanamine hydrochloride (0.103 g, 0.614 mmol) was added followed by propylphosphonic anhydride solution (50% w/w in DMF, 1.1 mL, 1.88 mmol) and pyridine (0.3 mL, 3.72 mmol) then heated to 65° C. for 3 days, then cooled, poured into water and filtered. The resulting solid was dissolved in a mixture of EtOAc, MeOH and acetone then concentrated onto silica gel and purified by flash chromatography (25-100% EtOAc in petroleum spirits), fractions containing the desired product were concentrated, dissolved in minimal DCM and purified by flash chromatography (50-75% EtOAc in DCM) to give rac-5-((5-(5-chloropyridin-2-yl)oxazol-2-yl)amino)-N-((2,2-dimethyl-1,3-dioxolan-4-yl)methyl)picolinamide (0.0248 g, 0.058 mmol, 6% yield from nitrile). ¹H NMR (401 MHz, MeOH-d4:CDCl₃ approx. 1:1 v/v) δ 8.66 (d, J=2.4 Hz, 1H), 8.43 (dd, J=2.4, 0.6 Hz, 1H), 8.20 (dd, J=8.6, 2.6 Hz, 1H), 8.06 (d, J=8.6 Hz, 1H), 7.72 (dd, J=8.6, 2.4 Hz, 1H), 7.53-7.47 (m, 2H), 4.35-4.28 (m, 1H), 4.06 (dd, J=8.5, 6.5 Hz, 1H), 3.69 (dd, J=8.5, 6.3 Hz, 1H), 3.62 (dd, J=14.0, 4.2 Hz, 1H), 3.52 (dd, J=14.0, 5.6 Hz, 1H), 1.43 (s, 3H), 1.32 (s, 3H). LCMS R_{f}(min)=3.55, MS m/z 430.1 [M+H]⁺.

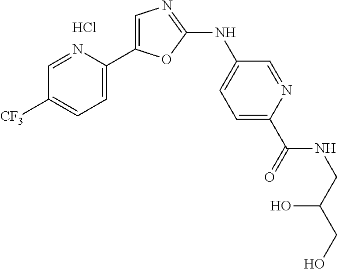 [SMILES: O=C(NCC(O)CO)c1ccc(Nc2ncc(-c3ccc(C(F)(F)F)cn3)o2)cn1 ; Cl]

rac-5-((5-(5-Chloropyridin-2-yl)oxazol-2-yl)amino)-N-((2,2-dimethyl-1,3-dioxolan-4-yl)methyl)picolinamide (0.0248 g, 0.058 mmol) was dissolved in 3 mL of a 1:1 v/v mixture of MeOH and DCM, HCl (1.25 M in MeOH, 0.2 mL, 0.25 mmol) was added and the mixture stirred at room temperature, after 1 h 1.5 mL of a 1.25 M HCl in MeOH solution (1.9 mmol) was added, after a further 3 h the reaction was concentrated, the residue suspended in 5 mL water, diluted with 5 mL MeOH and filtered and the solid was then washed with Et₂O and DCM to give rac-5-((5-(5-chloropyridin-2-yl)oxazol-2-yl)amino)-N-(2,3-dihydroxypropyl)picolinamide hydrochloride as a yellow solid (14.9 mg, 0.0350 mmol, 61%) in 90% purity as determined by HPLC (254 nm). The filtrate was left stand overnight after which time a precipitate formed which was collected by filtration and washed with water and let dry to give the title compound (1.6 mg, 0.004 mmol, 7%) in 98% purity as determined by HPLC (254 nm). ¹H NMR (401 MHz, DMSO-d₆) δ 11.20 (s, 1H), 8.79 (d, J=2.5 Hz, 1H), 8.64-8.61 (m, 1H), 8.45 (t, J=5.9 Hz, 1H), 8.30 (dd, J=8.6, 2.6 Hz, 1H), 8.06-7.99 (m, 2H), 7.75 (s, 1H), 7.65 (d, J=8.6 Hz, 1H), 4.93 (d, J=5.0 Hz, 1H), 4.64 (t, J=5.7 Hz, 1H), 3.66-3.56 (m, 1H), 3.54-3.45 (m, 1H), 3.42-3.35 (m, 2H)*, 3.26-3.17 (m, 1H). *overlapped with H₂O signal. LCMS R_{f}(min)=3.12, MS m/z 390.1 [M+H]⁺. HRMS (ESI) calcd for C₃₄H₃₂N₁₀O₈K [2 M+K]⁺ 747.2036, found 747.2034.

### 117. 5-((5-(5-Fluoropyridin-2-yl)oxazol-2-yl)amino)-N′-hydroxypicolinimidamide (Scheme 114)

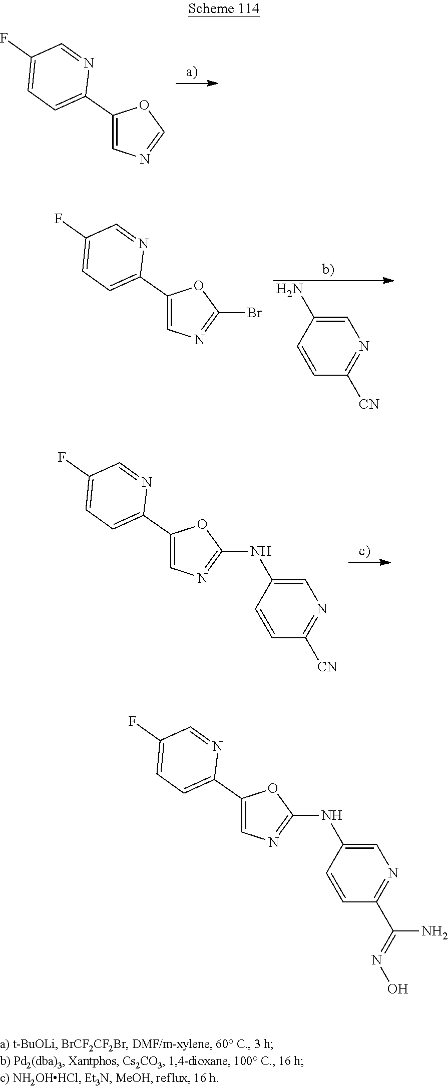 [SMILES: Fc1ccc(-c2cnco2)nc1 ; Fc1ccc(-c2cnc(Br)o2)nc1 ; N#Cc1ccc(N)cn1 ; N#Cc1ccc(Nc2ncc(-c3ccc(F)cn3)o2)cn1 ; N/C(=N\O)c1ccc(Nc2ncc(-c3ccc(F)cn3)o2)cn1]

5-(5-Fluoropyridin-2-yl)oxazole (0.40 g, 2.45 mmol) was reacted with BrCF₂CF₂Br (0.58 mL, 4.90 mmol) and t-BuOLi (0.392 g, 4.90 mmol) in DMF/m-xylene (4/4 mL) at 60° C. for 3 h as per General Procedure 15 Method 2 to provide 2-bromo-5-(5-fluoropyridin-2-yl)oxazole (0.28 g, 46%) as a yellow solid. ¹H NMR (401 MHz, CDCl₃) δ 8.46 (d, J=2.8 Hz, 1H), 7.62 (dd, J=8.7, 4.3 Hz, 1H), 7.56 (s, 1H), 7.47 (ddd, J=8.7, 8.0, 2.9 Hz, 1H). LCMS R_{f}(min)=3.446, MS m/z=245.0 [M+H]⁺.

5-Aminonicotinonitrile (280 mg, 2.35 mmol, 3.0 eq.), Cs₂CO₃ (764 mg, 2.35 mmol, 3.0 eq.) and 2-bromo-5-(5-fluoropyridin-2-yl)oxazole (190 mg, 0.782 mmol, 1.0 eq.) in 1,4-dioxane (8 mL) was charged with Pd₂(dba)₃ (36 mg, 0.039 mmol, 0.05 eq.), Xantphos (45 mg, 0.078 mmol, 0.1 eq.) and reacted according to General Procedure 4 Method 1 to afford 5-((5-(5-fluoropyridin-2-yl)oxazol-2-yl)amino)picolinonitrile as yellow solid (190 mg, 83%). LCMS R_{f} (min)=3.215, MS m/z=282.1 [M+H]⁺.

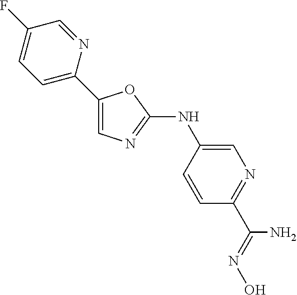 [SMILES: N/C(=N\O)c1ccc(Nc2ncc(-c3ccc(F)cn3)o2)cn1]

5-((5-(5-Fluoropyridin-2-yl)oxazol-2-yl)amino)picolinonitrile (180 mg, 0.64 mmol, 1.0 eq.) was suspended in EtOH (15 mL) followed by the addition of NH₂OH·HCl (356 mg, 5.12 mmol, 8.0 eq.) and Et₃N (714 μL, 5.12 mmol, 8.0 eq.) and reacted according to General Procedure 1 Method 2 to afford 5-((5-(5-fluoropyridin-2-yl)oxazol-2-yl)amino)-N′-hydroxypicolinimidamide as beige solid (40 mg, 20%). ¹H NMR (401 MHz, DMSO-d₆) δ 10.92 (br s, 1H), 9.75 (br s, 1H), 8.77 (br s, 1H), 8.58 (s, 1H), 8.12 (br s, 1H), 7.83 (br s, 2H), 7.63 (br s, 2H), 5.76 (br s, 2H). LCMS R_{f} (min)=2.999, MS m/z=315.1 [M+H]⁺. HRMS (ESI) calcd for C₁₄H₁₂FN₆O₂⁺ ([M+H]⁺ 315.1000, found 315.1004.

### 118. N′-Hydroxy-5-((5-(4-(trifluoromethoxy)phenyl)oxazol-2-yl)amino)picolinimidamide (Scheme 115)

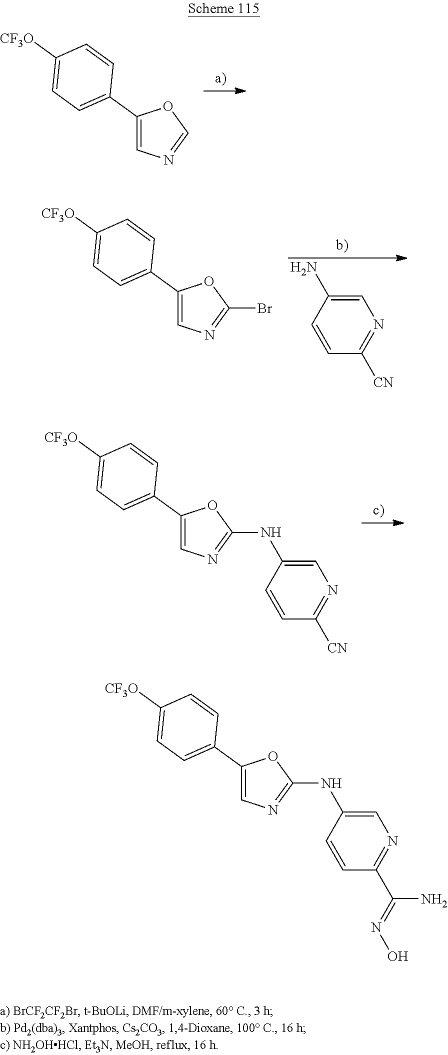

5-(4-(Trifluoromethoxy)phenyl)oxazole (0.9 g, 3.93 mmol) was reacted with BrCF₂CF₂Br (0.70 mL, 5.90 mmol) and t-BuOLi (0.409 g, 5.11 mmol) in DMF/m-xylene (5/5 mL) as per General Procedure 15 Method 2 to provide 2-bromo-5-(4-(trifluoromethoxy)phenyl)oxazole (0.676 g, 56%) as an orange solid. ¹H NMR (401 MHz, CDCl₃) δ 7.62-7.54 (m, 2H), 7.26 (s, 1H, overlapping with CHCl₃ residue), 7.25-7.23 (m, 2H). LCMS R_{f}(min)=3.752, MS m/z=309.9 [M+H]⁺.

5-Aminonicotinonitrile (232 mg, 1.95 mmol, 3.0 eq.), Cs₂CO₃ (634 mg, 1.95 mmol, 3.0 eq.) and 2-bromo-5-(4-(trifluoromethoxy)phenyl)oxazole (200 mg, 0.649 mmol, 1.0 eq.) in 1,4-dioxane (8 mL) was charged with Pd₂(dba)₃ (30 mg, 0.033 mmol, 0.05 eq.) and Xantphos (37 mg, 0.064 mmol, 0.1 eq.) and reacted according to General Procedure 4 Method 1 to afford 5-((5-(4-(trifluoromethoxy)phenyl)oxazol-2-yl)amino)picolinonitrile as yellow solid (160 mg, 71%). ¹H NMR (401 MHz, DMSO-d₆) δ 11.37 (s, 1H), 8.87 (d, J=2.5 Hz, 1H), 8.36 (dd, J=8.7, 2.7 Hz, 1H), 8.02 (d, J=8.7 Hz, 1H), 7.84-7.73 (m, 2H), 7.69 (s, 1H), 7.50 (d, J=8.1 Hz, 2H). LCMS R_{f} (min)=3.935, MS m/z=347.0 [M+H]⁺.

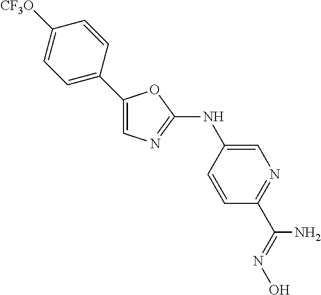

5-((5-(4-(Trifluoromethoxy)phenyl)oxazol-2-yl)amino)picolinonitrile (70 mg, 0.202 mmol, 1.0 eq.) was suspended in absolute MeOH (8 mL) followed by the addition of NH₂OH·HCl (112 mg, 1.61 mmol, 8.0 eq.) and Et₃N (226 μL, 1.61 mmol, 8.0 eq.) and reacted according to General Procedure 1 Method 2 to afford N′-hydroxy-5-((5-(4-(trifluoromethoxy)phenyl)oxazol-2-yl)amino)picolinimidamide as beige solid (30 mg, 39%). ¹H NMR (401 MHz, DMSO-d₆) δ 10.80 (s, 1H), 9.73 (s, 1H), 8.77 (d, J=2.3 Hz, 1H), 8.12 (dd, J=8.8, 2.5 Hz, 1H), 7.83 (d, J=8.8 Hz, 1H), 7.72 (d, J=8.7 Hz, 2H), 7.60 (s, 1H), 7.46 (d, J=8.3 Hz, 2H), 5.75 (s, 2H); ¹³C NMR (101 MHz, DMSO-d₆) δ 156.1, 149.4, 147.1, 143.2, 143.0, 136.7, 136.3, 127.2, 124.4, 123.8, 123.6, 121.9, 121.4, 119.7, 118.8. LCMS R_{f}(min)=3.294, MS m/z=380.1 [M+H]⁺. HRMS (ESI) calcd for C₁₆H₁₃F₃N₅O₃⁺ ([M+H]⁺ 380.0965, found 380.0975.

### 119. N′-Hydroxy-5-((5-(6-(trifluoromethyl)pyridazin-3-yl)oxazol-2-yl)amino)picolinimidamide (Scheme 116)

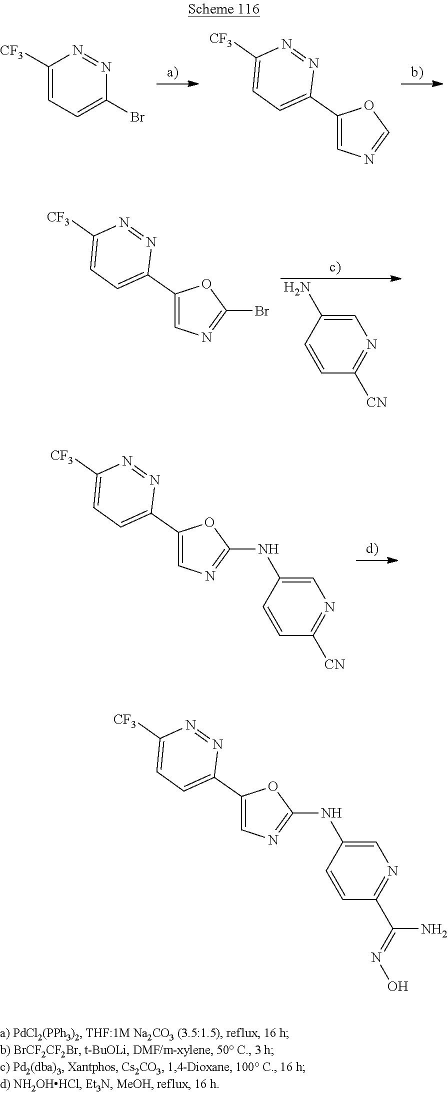 [SMILES: FC(F)(F)c1ccc(Br)nn1 ; FC(F)(F)c1ccc(-c2cnco2)nn1 ; FC(F)(F)c1ccc(-c2cnc(Br)o2)nn1 ; N#Cc1ccc(N)cn1 ; N#Cc1ccc(Nc2ncc(-c3ccc(C(F)(F)F)nn3)o2)cn1 ; N/C(=N\O)c1ccc(Nc2ncc(-c3ccc(C(F)(F)F)nn3)o2)cn1]

To a degassed biphasic solution of THF (3.5 mL) and 1 M Na₂CO₃ (1.5 mL), was added 3-chloro-6-(trifluoromethyl)pyridazine (150 mg, 0.822 mmol, 1.0 eq.), 5-(4,4,5,5-tetramethyl-1,3,2-dioxaborolan-2-yl)oxazole (1.0 M in THF, 904 μL, 0.904 mmol, 1.1 eq.) and PdCl₂(PPh₃)₂ (58 mg, 0.082 mmol, 0.1 eq.). The mixture was reacted according to General Procedure 12 Method 1 to afford 5-(6-(trifluoromethyl)pyridazin-3-yl)oxazole as a brown solid (99 mg, 55%). ¹H NMR (401 MHz, CDCl₃) δ 8.12 (m, 2H), 7.98 (d, J=8.9 Hz, 1H), 7.90 (d, J=8.8 Hz, 1H); ¹³C NMR (101 MHz, CDCl₃) δ 152.9, 152.4, 150.7, 150.4, 147.7, 125.4, 124.6, 124.6, 124.5, 124.5, 122.9, 122.9, 122.7, 120.0. LCMS R_{f}(min)=3.223, MS m/z=216.0 [M+H]⁺.

5-(6-(Trifluoromethyl)pyridazin-3-yl)oxazole (0.322 g, 1.50 mmol) was reacted with BrCF₂CF₂Br (0.27 mL, 2.25 mmol) and t-BuOLi (0.156 g, 1.94 mmol) in DMF/m-xylene (3/3 mL) at 50° C. for 3 h as per General Procedure 15 Method 2 to provide 2-bromo-5-(6-(trifluoromethyl)pyridazin-3-yl)oxazole (0.221 g, 50%) as an orange solid. ¹H NMR (401 MHz, CDCl₃) δ 8.04 (s, 1H), 7.96-7.90 (m, 2H). LCMS R_{f}(min)=3.033, MS m/z=295.9 [M+H]⁺.

5-Aminonicotinonitrile (110 mg, 0.923 mmol, 3.0 eq.), Cs₂CO₃ (301 mg, 0.924 mmol, 3.0 eq.) and 2-bromo-5-(6-(trifluoromethyl)pyridazin-3-yl)oxazole (90 mg, 0.307 mmol, 1.0 eq.) in 1,4-dioxane (5 mL) was charged with Pd₂(dba)₃ (14 mg, 0.015 mmol, 0.05 eq.), Xantphos (18 mg, 0.031 mmol, 0.1 eq.) and reacted according to General Procedure 4 Method 1 to afford 5-((5-(6-(trifluoromethyl)pyridazin-3-yl)oxazol-2-yl)amino)picolinonitrile as yellow solid (50 mg, 49%). ¹H NMR (401 MHz, DMSO) δ 11.77 (s, 1H), 8.87 (s, 1H), 8.39-8.32 (m, 1H), 8.30 (dd, J=9.1, 4.6 Hz, 1H), 8.23 (dd, J=8.9, 4.3 Hz, 2H), 8.02 (dd, J=8.6, 4.4 Hz, 1H) ppm. LCMS R_{f}(min)=3.359, MS m/z=333.0 [M+H]⁺.

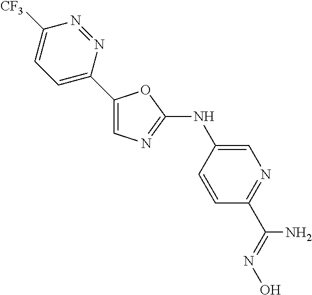 [SMILES: N/C(=N\O)c1ccc(Nc2ncc(-c3ccc(C(F)(F)F)nn3)o2)cn1]

5-((5-(6-(Trifluoromethyl)pyridazin-3-yl)oxazol-2-yl)amino)picolinonitrile (40 mg, 0.12 mmol, 1.0 eq.) was suspended in absolute MeOH (4 mL) followed by the addition of NH₂OH·HCl (67 mg, 0.964 mmol, 8.0 eq.) and Et₃N (134 μL, 0.964 mmol, 8.0 eq.) according to General Procedure 1 Method 2 to afford N′-hydroxy-5-((5-(6-(trifluoromethyl)pyridazin-3-yl)oxazol-2-yl)amino)picolinimidamide as beige solid (30 mg, 68%). ¹H NMR (401 MHz, DMSO-d₆) δ 11.27 (s, 1H), 9.77 (s, 1H), 8.81 (s, 1H), 8.28 (d, J=9.2 Hz, 1H), 8.21-8.14 (m, 3H), 7.86 (d, J=8.8 Hz, 1H), 5.78 (s, 2H); ¹³C NMR (101 MHz, DMSO) δ 158.4, 151.7, 149.3, 148.3, 148.0, 143.6, 140.7, 137.1, 135.8, 131.7, 125.1, 124.3, 123.1, 119.8 ppm. LCMS R_{f}(min)=2.283, MS m/z=366.1 [M+H]⁺. HRMS (ESI) calcd for C₁₄H₁₁F₃N₇O₂⁺ ([M+H]⁺ 366.0921, found 366.0935.

### 120. 5-((5-(4-Cyclopropylphenyl)oxazol-2-yl)amino)-N′-hydroxypicolinimidamide (Scheme 117)

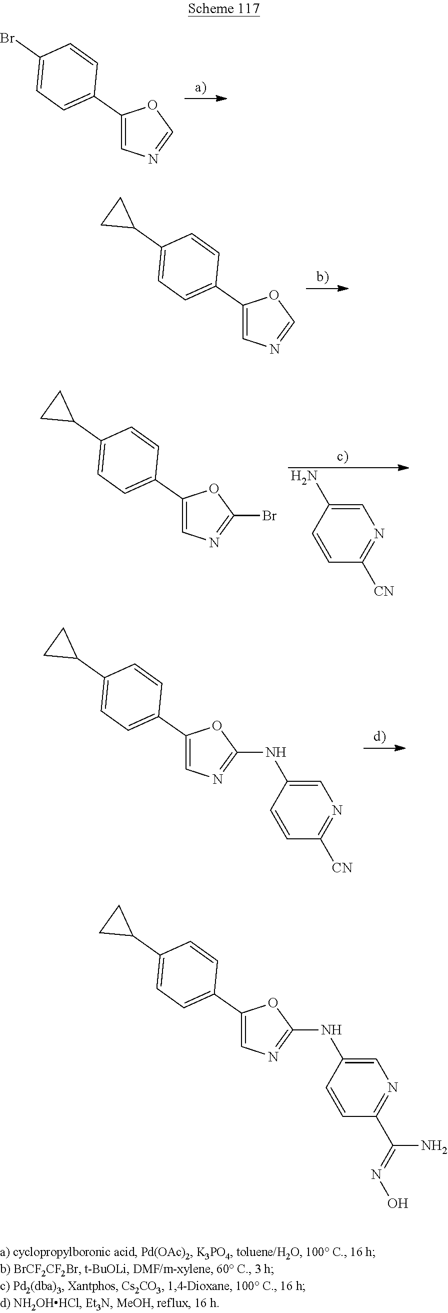 [SMILES: Brc1ccc(-c2cnco2)cc1 ; c1ncc(-c2ccc(C3CC3)cc2)o1 ; Brc1ncc(-c2ccc(C3CC3)cc2)o1 ; N#Cc1ccc(N)cn1 ; N#Cc1ccc(Nc2ncc(-c3ccc(C4CC4)cc3)o2)cn1 ; N/C(=N\O)c1ccc(Nc2ncc(-c3ccc(C4CC4)cc3)o2)cn1]

5-(4-Bromophenyl)oxazole (1.53 g, 6.83 mmol) was reacted with cyclopropylboronic acid (0.76, 8.88 mmol), potassium phosphate (4.35 g, 20.49 mmol), Pd(OAc)₂ (0.077 g, 0.34 mmol) and tricyclohexylphosphine (0.192 g, 0.68 mmol) in toluene/H₂O (40/4 mL) as per General Procedure 12 Method 2 to provide 5-(4-cyclopropylphenyl)oxazole (1.26 g, 89%) as a white solid. ¹H NMR (401 MHz, CDCl₃) δ 7.88 (s, 1H), 7.53 (d, J=8.3 Hz, 2H), 7.28 (s, 1H), 7.11 (d, J=8.3 Hz, 2H), 1.91 (m, 1H), 1.19-0.92 (m, 2H), 0.84-0.58 (m, 2H). LCMS R_{f}(min)=3.461, MS n/z=186.1 [M+H]⁺.

5-(4-Cyclopropylphenyl)oxazole (0.60 g, 3.24 mmol) was reacted with BrCF₂CF₂Br (0.58 mL, 4.86 mmol) and t-BuOLi (0.337 g, 4.21 mmol) in DMF/m-xylene (8/8 mL) at 60° C. for 3 h as per General Procedure 15 Method 2 to provide 2-bromo-5-(4-cyclopropylphenyl)oxazole (0.419 g, 49%) as a yellow solid. ¹H NMR (401 MHz, CDCl₃) δ 7.48 (d, J=8.4 Hz, 2H), 7.22 (s, 1H), 7.11 (d, J=8.3 Hz, 2H), 1.92 (m, 1H), 1.02 (ddd, J=8.4, 6.5, 4.6 Hz, 2H), 0.73 (dt, J=6.6, 4.7 Hz, 2H). LCMS R_{f} (min)=3.703, MS m/z=264.0 [M+H]⁺.

5-Aminonicotinonitrile (271 mg, 2.27 mmol, 3.0 eq.), Cs₂CO₃ (741 mg, 2.27 mmol, 3.0 eq.) and 2-bromo-5-(5-(trifluoromethyl)pyrazin-2-yl)oxazole (200 mg, 0.757 mmol, 1.0 eq.) in 1,4-dioxane (8 mL) was charged with Pd₂(dba)₃ (35 mg, 0.038 mmol, 0.05 eq.) and Xantphos (44 mg, 0.076 mmol, 0.1 eq.) and reacted according to General Procedure 4 Method 1 to afford 5-((5-(4-cyclopropylphenyl)oxazol-2-yl)amino)picolinonitrile as yellow solid (150 mg, 66%). ¹H NMR (401 MHz, DMSO-d₆) δ 11.25 (s, 1H), 8.82 (s, 1H), 8.32 (d, J=6.6 Hz, 1H), 7.98 (d, J=8.7 Hz, 1H), 7.51 (s, 1H), 7.49 (s, 2H), 7.16 (d, J=8.0 Hz, 2H), 1.94 (m, 1H), 0.97 (m, 2H), 0.71 (m, 2H). LCMS R_{f}(min)=3.256, MS m/z=303.1 [M+H]⁺.

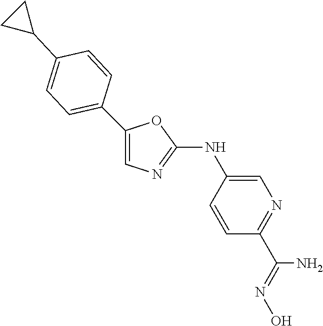 [SMILES: N/C(=N\O)c1ccc(Nc2ncc(-c3ccc(C4CC4)cc3)o2)cn1]

5-((5-(4-Cyclopropylphenyl)oxazol-2-yl)amino) picolinonitrile (75 mg, 0.248 mmol, 1.0 eq.) was suspended in absolute MeOH (6 mL) followed by the addition of NH₂OH·HCl (138 mg, 1.99 mmol, 8.0 eq.) and Et₃N (201 mg, 1.99 mmol, 8.0 eq.) and reacted according to General Procedure 1 Method 2 to afford 5-((5-(4-cyclopropylphenyl)oxazol-2-yl)amino)-N′-hydroxypicolinimidamide as beige solid (40 mg, 48%). ¹H NMR (401 MHz, DMSO-d₆) δ 10.71 (s, 1H), 9.72 (s, 1H), 8.76 (s, 1H), 8.10 (m, 1H), 7.83 (m, 1H), 7.50-7.23 (m, 3H), 7.15 (m, 2H), 5.75 (s, 2H), 1.94 (m, 1H), 0.97 (m, 2H), 0.70 (m, 2H); ¹³C NMR (101 MHz, DMSO) δ 155.5, 149.4, 144.5, 143.2, 142.7, 136.5, 136.4, 125.9, 124.9, 123.5, 122.7, 121.6, 119.7, 15.0, 9.6 ppm. LCMS R_{f} (min)=2.519, MS m/z=336.1 [M+H]⁺. HRMS (ESI) calcd for C₁₈H₁₈N₅O₂⁺ ([M+H]⁺ 336.1455, found 336.1464.

### Example 2

Measurement of dihydroceramide desaturase-1 (Des-1) activity Compounds were assessed for Des-1 inhibitory activity. Measurement of Des1 activity was performed by HPLC using intact Jurkat cells labeled with DhCer-C6-NBD as described previously (Munoz-Olaya, J. M. et al. ChemMedChem 2008, 3, 946-953) with modifications to enhance sensitivity and reproducibility. These modifications included the use of parental Jurkat cells, 0.5% serum in the culture media, and cell harvesting via centrifugation at 500×g to maximise ceramide extraction. Extracted samples (50 ul) were analysed on a Waters HPLC coupled to a fluorescence detector using a 30 cm C18 reverse-phase column eluted with 1 ml/min 20% H₂O and 80% acetonitrile, both with a 0.1% of trifluoroacetic acid. NBD-labelled substrate and product were quantitated with an excitation and emission wavelengths of 465 nm and 530 nm, respectively.

The results are presented in Table 2-1.

TABLE 2-1

| | | Des1 (h) Jurkat |
|---|---|---|
| Compound | Structure | IC₅₀ ^{a} μM |
| 1 | 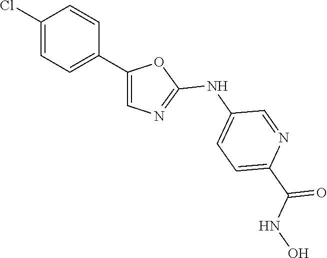 [SMILES: O=C(NO)c1ccc(Nc2ncc(-c3ccc(Cl)cc3)o2)cn1] | 0.1-1.0 |
| 2 | 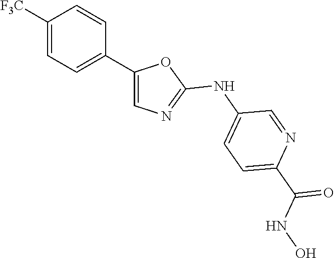 [SMILES: O=C(NO)c1ccc(Nc2ncc(-c3ccc(C(F)(F)F)cc3)o2)cn1] | 0.1-1.0 |
| 3 | 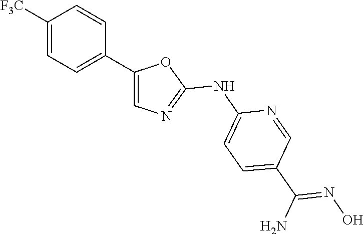 [SMILES: N/C(=N\O)c1ccc(Nc2ncc(-c3ccc(C(F)(F)F)cc3)o2)nc1] | 0.1-1.0 |
| 4 | 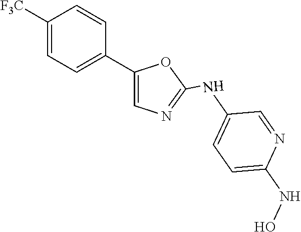 [SMILES: ONc1ccc(Nc2ncc(-c3ccc(C(F)(F)F)cc3)o2)cn1] | 1.0-10 |
| 5 | 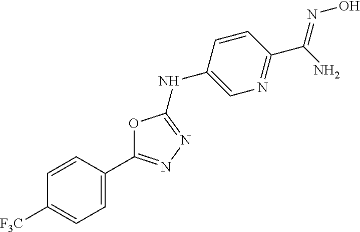 [SMILES: N/C(=N\O)c1ccc(Nc2nnc(-c3ccc(C(F)(F)F)cc3)o2)cn1] | 0.1-1.0 |
| 6 | 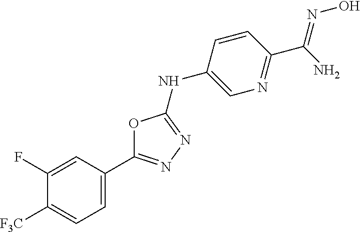 [SMILES: N/C(=N\O)c1ccc(Nc2nnc(-c3ccc(C(F)(F)F)c(F)c3)o2)cn1] | 0.1-1.0 |
| 7 | 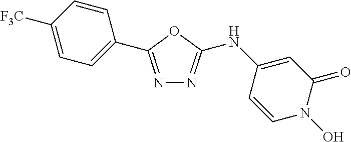 [SMILES: O=c1cc(Nc2nnc(-c3ccc(C(F)(F)F)cc3)o2)ccn1O] | <1 |
| 8 | 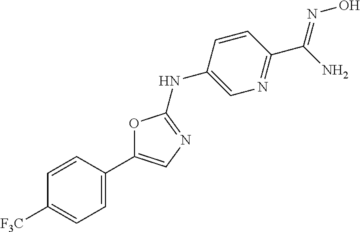 [SMILES: N/C(=N\O)c1ccc(Nc2ncc(-c3ccc(C(F)(F)F)cc3)o2)cn1] | 0.1-1.0 |
| 9 | 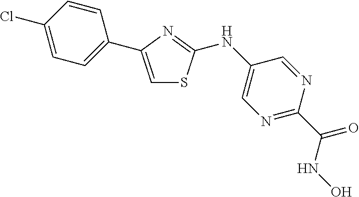 [SMILES: O=C(NO)c1ncc(Nc2nc(-c3ccc(Cl)cc3)cs2)cn1] | >10 (26% inhibition at 10 μM) |
| 10 | 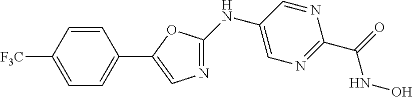 [SMILES: O=C(NO)c1ncc(Nc2ncc(-c3ccc(C(F)(F)F)cc3)o2)cn1] | 1.0-10 |
| 11 | 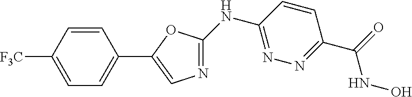 [SMILES: O=C(NO)c1ccc(Nc2ncc(-c3ccc(C(F)(F)F)cc3)o2)nn1] | 0.1-1.0 |
| 12 | 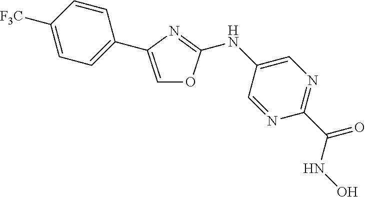 [SMILES: O=C(NO)c1ncc(Nc2nc(-c3ccc(C(F)(F)F)cc3)co2)cn1] | 1.0-10 |
| 13 | 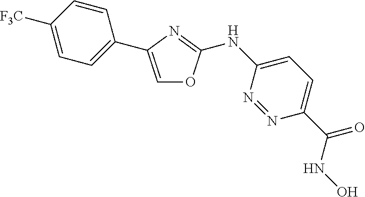 [SMILES: O=C(NO)c1ccc(Nc2nc(-c3ccc(C(F)(F)F)cc3)co2)nn1] | 1.0-10 |
| 14 | 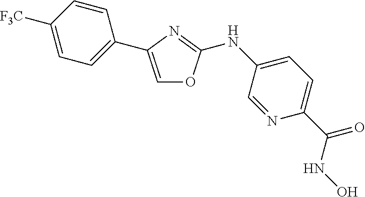 [SMILES: O=C(NO)c1ccc(Nc2nc(-c3ccc(C(F)(F)F)cc3)co2)cn1] | 0.1-1.0 |
| 15 | 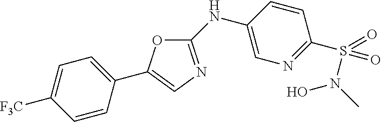 [SMILES: CN(O)S(=O)(=O)c1ccc(Nc2ncc(-c3ccc(C(F)(F)F)cc3)o2)cn1] | >1 (46% inhibition at 1 μM) |
| 16 | 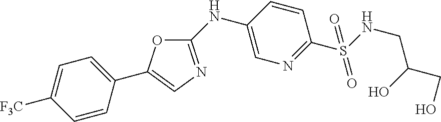 [SMILES: O=S(=O)(NCC(O)CO)c1ccc(Nc2ncc(-c3ccc(C(F)(F)F)cc3)o2)cn1] | >1 (42% inhibition at 1 μM) |
| 17 | 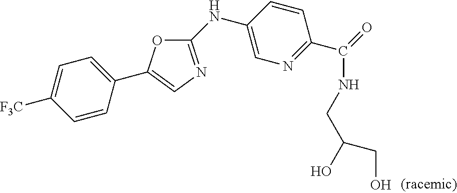 [SMILES: O=C(NCC(O)CO)c1ccc(Nc2ncc(-c3ccc(C(F)(F)F)cc3)o2)cn1] | 0.001-0.01 |
| 18 | 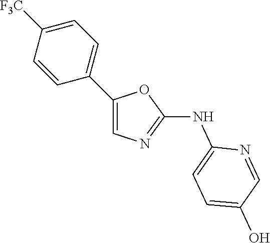 [SMILES: Oc1ccc(Nc2ncc(-c3ccc(C(F)(F)F)cc3)o2)nc1] | <1 |
| 19 | 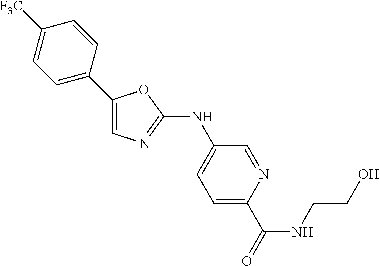 [SMILES: O=C(NCCO)c1ccc(Nc2ncc(-c3ccc(C(F)(F)F)cc3)o2)cn1] | 1.0-10 |
| 20 | 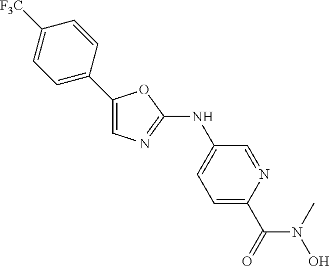 [SMILES: CN(O)C(=O)c1ccc(Nc2ncc(-c3ccc(C(F)(F)F)cc3)o2)cn1] | <0.1 |
| 21 | 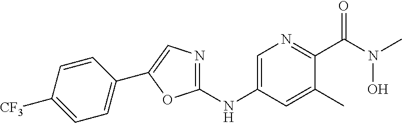 [SMILES: Cc1cc(Nc2ncc(-c3ccc(C(F)(F)F)cc3)o2)cnc1C(=O)N(C)O] | >1 (38% inhibition at 1 μM) |
| 22 | 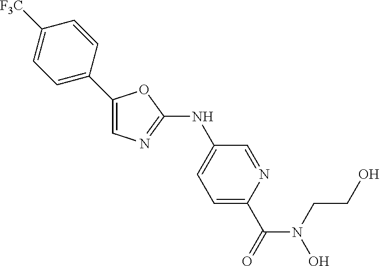 [SMILES: O=C(c1ccc(Nc2ncc(-c3ccc(C(F)(F)F)cc3)o2)cn1)N(O)CCO] | >10 (81% inhibition at 10 μM) |
| 23 | 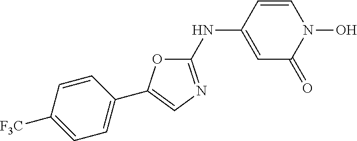 [SMILES: O=c1cc(Nc2ncc(-c3ccc(C(F)(F)F)cc3)o2)ccn1O] | 0.001-0.01 |
| 24 | 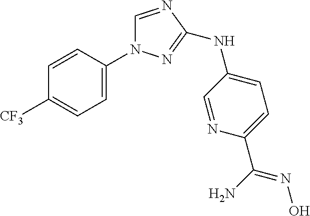 [SMILES: N/C(=N\O)c1ccc(Nc2ncn(-c3ccc(C(F)(F)F)cc3)n2)cn1] | 0.01-0.1 |
| 25 | 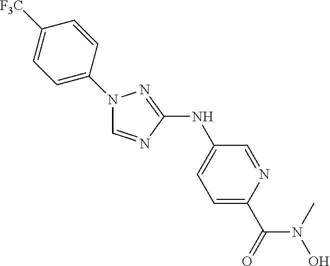 [SMILES: CN(O)C(=O)c1ccc(Nc2ncn(-c3ccc(C(F)(F)F)cc3)n2)cn1] | <1 |
| 26 | 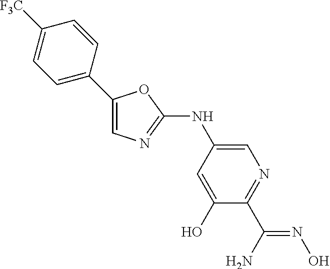 [SMILES: N/C(=N\O)c1ncc(Nc2ncc(-c3ccc(C(F)(F)F)cc3)o2)cc1O] | 0.001-0.01 |
| 27 | 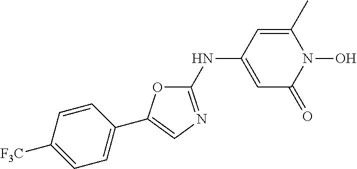 [SMILES: Cc1cc(Nc2ncc(-c3ccc(C(F)(F)F)cc3)o2)cc(=O)n1O] | 0.0001-0.001 |
| 28 | 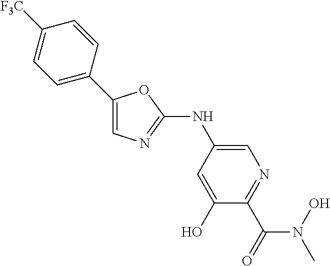 [SMILES: CN(O)C(=O)c1ncc(Nc2ncc(-c3ccc(C(F)(F)F)cc3)o2)cc1O] | <1 |
| 29 | 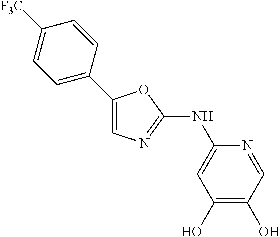 [SMILES: Oc1cnc(Nc2ncc(-c3ccc(C(F)(F)F)cc3)o2)cc1O] | <1 |
| 30 | 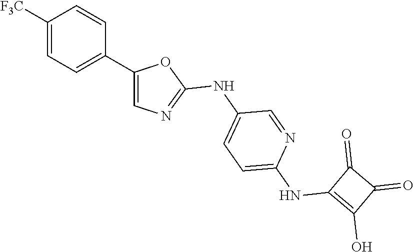 [SMILES: O=c1c(O)c(Nc2ccc(Nc3ncc(-c4ccc(C(F)(F)F)cc4)o3)cn2)c1=O] | <1 |
| 31 | 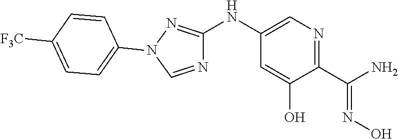 [SMILES: N/C(=N\O)c1ncc(Nc2ncn(-c3ccc(C(F)(F)F)cc3)n2)cc1O] | <1 |
| 32 | 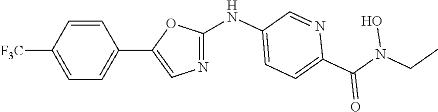 [SMILES: CCN(O)C(=O)c1ccc(Nc2ncc(-c3ccc(C(F)(F)F)cc3)o2)cn1] | <1 |
| 33 | 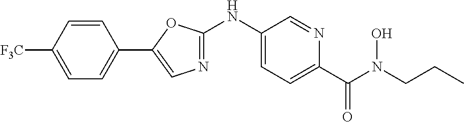 [SMILES: CCCN(O)C(=O)c1ccc(Nc2ncc(-c3ccc(C(F)(F)F)cc3)o2)cn1] | 1 |
| 34 | 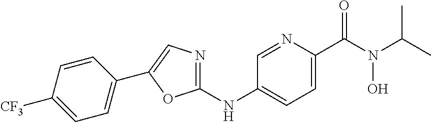 [SMILES: CC(C)N(O)C(=O)c1ccc(Nc2ncc(-c3ccc(C(F)(F)F)cc3)o2)cn1] | >1 (23% inhibition at 1 μM) |
| 35 | 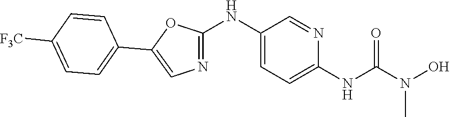 [SMILES: CN(O)C(=O)Nc1ccc(Nc2ncc(-c3ccc(C(F)(F)F)cc3)o2)cn1] | 0.01-0.1 |
| 36 | 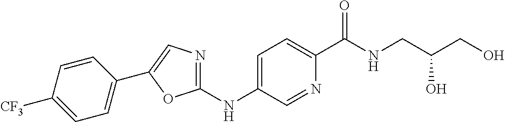 [SMILES: O=C(NC[C@@H](O)CO)c1ccc(Nc2ncc(-c3ccc(C(F)(F)F)cc3)o2)cn1] | 0.001-0.01 |
| 37 | 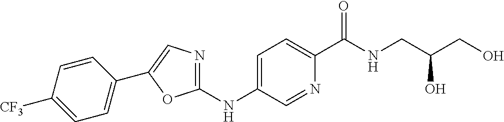 [SMILES: O=C(NC[C@H](O)CO)c1ccc(Nc2ncc(-c3ccc(C(F)(F)F)cc3)o2)cn1] | >1 (no inhibition at 1 μM) |
| 38 | 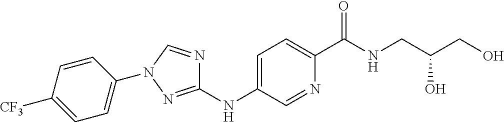 [SMILES: O=C(NC[C@@H](O)CO)c1ccc(Nc2ncn(-c3ccc(C(F)(F)F)cc3)n2)cn1] | >1 (30% inhibition at 1 μM) |
| 39 | 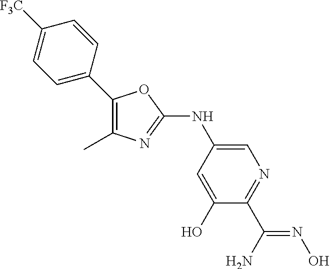 [SMILES: Cc1nc(Nc2cnc(/C(N)=N/O)c(O)c2)oc1-c1ccc(C(F)(F)F)cc1] | <1 |
| 40 | 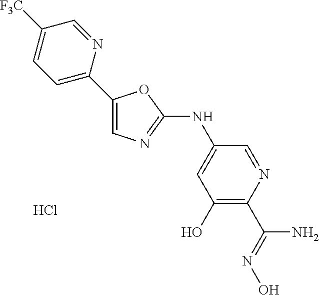 [SMILES: N/C(=N\O)c1ncc(Nc2ncc(-c3ccc(C(F)(F)F)cn3)o2)cc1O ; Cl] | 0.01-0.1 |
| 41 | 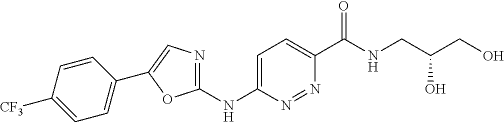 [SMILES: O=C(NC[C@@H](O)CO)c1ccc(Nc2ncc(-c3ccc(C(F)(F)F)cc3)o2)nn1] | <0.1 |
| 42 | 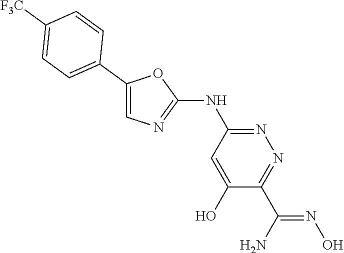 [SMILES: N/C(=N\O)c1nnc(Nc2ncc(-c3ccc(C(F)(F)F)cc3)o2)cc1O] | 0.001-0.01 |
| 43 | 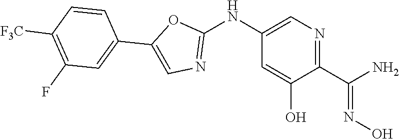 [SMILES: N/C(=N\O)c1ncc(Nc2ncc(-c3ccc(C(F)(F)F)c(F)c3)o2)cc1O] | 0.001-0.01 |
| 44 | 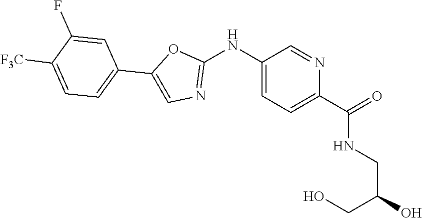 [SMILES: O=C(NC[C@@H](O)CO)c1ccc(Nc2ncc(-c3ccc(C(F)(F)F)c(F)c3)o2)cn1] | 0.001-0.01 |
| 45 | 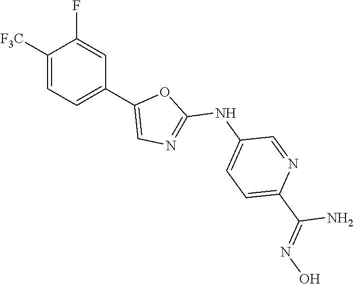 [SMILES: N/C(=N\O)c1ccc(Nc2ncc(-c3ccc(C(F)(F)F)c(F)c3)o2)cn1] | <0.1 |
| 46 | 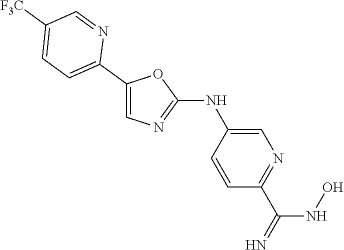 [SMILES: N=C(NO)c1ccc(Nc2ncc(-c3ccc(C(F)(F)F)cn3)o2)cn1] | 0.1-1.0 |
| 47 | 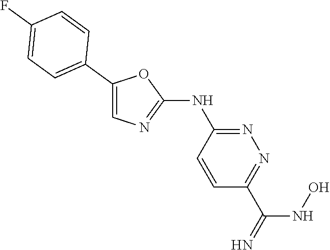 [SMILES: N=C(NO)c1ccc(Nc2ncc(-c3ccc(F)cc3)o2)nn1] | <0.1 |
| 48 | 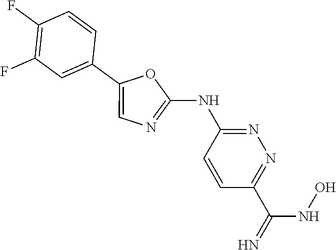 [SMILES: N=C(NO)c1ccc(Nc2ncc(-c3ccc(F)c(F)c3)o2)nn1] | 0.001-0.01 |
| 49 | 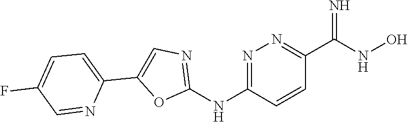 [SMILES: N=C(NO)c1ccc(Nc2ncc(-c3ccc(F)cn3)o2)nn1] | <0.1 |
| 50 | 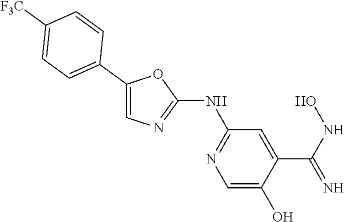 [SMILES: N=C(NO)c1cc(Nc2ncc(-c3ccc(C(F)(F)F)cc3)o2)ncc1O] | <1 |
| 51 | 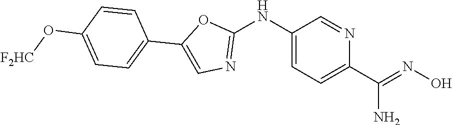 [SMILES: N/C(=N\O)c1ccc(Nc2ncc(-c3ccc(OC(F)F)cc3)o2)cn1] | 0.1-1.0 |
| 52 | 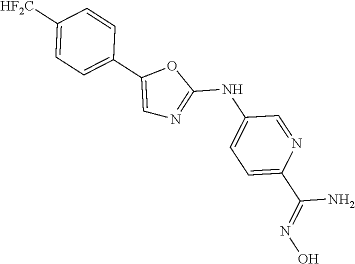 | 0.1-1.0 |
| 53 | 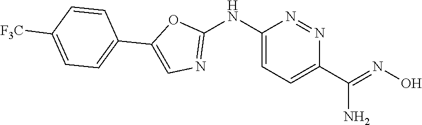 [SMILES: N/C(=N\O)c1ccc(Nc2ncc(-c3ccc(C(F)(F)F)cc3)o2)nn1] | 0.001-0.01 |
| 54 | 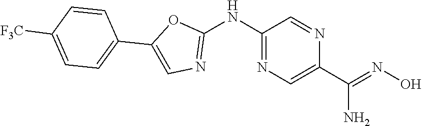 [SMILES: N/C(=N\O)c1cnc(Nc2ncc(-c3ccc(C(F)(F)F)cc3)o2)cn1] | 0.1-1.0 |
| 55 | 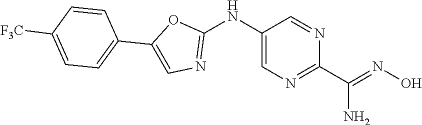 [SMILES: N/C(=N\O)c1ncc(Nc2ncc(-c3ccc(C(F)(F)F)cc3)o2)cn1] | <1 |
| 56 | 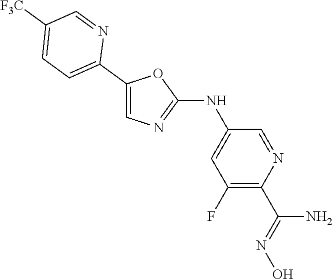 [SMILES: N/C(=N\O)c1ncc(Nc2ncc(-c3ccc(C(F)(F)F)cn3)o2)cc1F] | >0.1 (86% inhibition at 0.1 μM) |
| 57 | 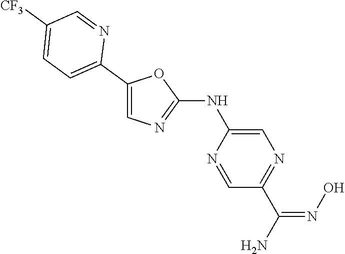 [SMILES: N/C(=N/O)c1cnc(Nc2ncc(-c3ccc(C(F)(F)F)cn3)o2)cn1] | <1 |
| 58 | 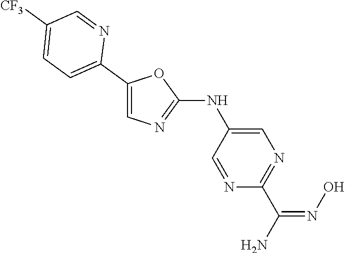 [SMILES: N/C(=N/O)c1ncc(Nc2ncc(-c3ccc(C(F)(F)F)cn3)o2)cn1] | >1 (40% inhibition at 1 μM) |
| 59 | 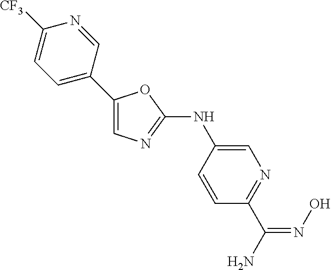 [SMILES: N/C(=N/O)c1ccc(Nc2ncc(-c3ccc(C(F)(F)F)nc3)o2)cn1] | <1 |
| 60 | 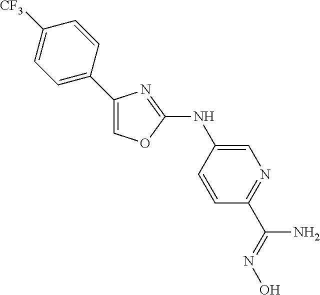 [SMILES: N/C(=N\O)c1ccc(Nc2nc(-c3ccc(C(F)(F)F)cc3)co2)cn1] | <1 |
| 61 | 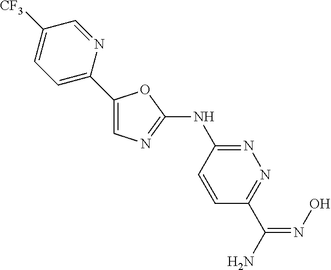 [SMILES: N/C(=N/O)c1ccc(Nc2ncc(-c3ccc(C(F)(F)F)cn3)o2)nn1] | <0.1 |
| 62 | 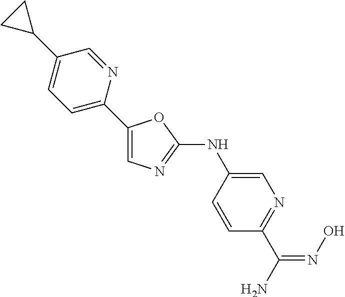 [SMILES: N/C(=N/O)c1ccc(Nc2ncc(-c3ccc(C4CC4)cn3)o2)cn1] | >1 (42% inhibition at 1 μM) |
| 63 | 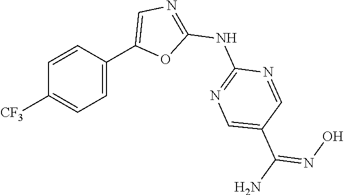 [SMILES: N/C(=N/O)c1cnc(Nc2ncc(-c3ccc(C(F)(F)F)cc3)o2)nc1] | >1 (23% inhibition at 1 μM) |
| 64 | 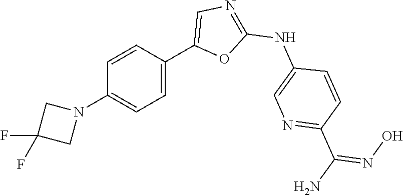 [SMILES: N/C(=N/O)c1ccc(Nc2ncc(-c3ccc(N4CC(F)(F)C4)cc3)o2)cn1] | >1 (23% inhibition at 1 μM) |
| 65 | 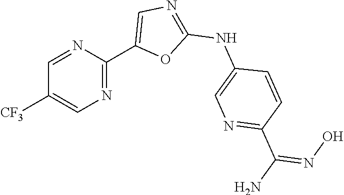 [SMILES: N/C(=N/O)c1ccc(Nc2ncc(-c3ncc(C(F)(F)F)cn3)o2)cn1] | >1 (26% inhibition at 1 μM) |
| 66 | 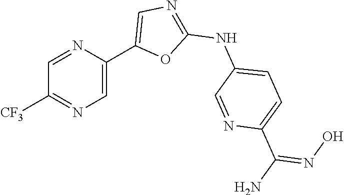 [SMILES: N/C(=N/O)c1ccc(Nc2ncc(-c3cnc(C(F)(F)F)cn3)o2)cn1] | >1 (47% inhibition at 1 μM) |
| 67 | 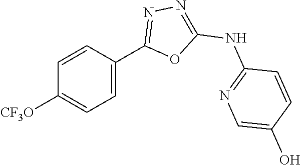 | <1 |
| 68 | 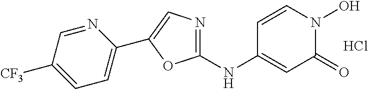 [SMILES: O=c1cc(Nc2ncc(-c3ccc(C(F)(F)F)cn3)o2)ccn1O ; Cl] | <1 |
| 69 | 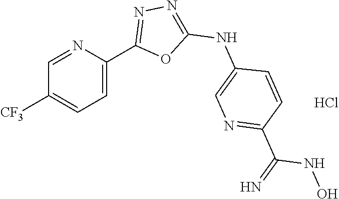 [SMILES: N=C(NO)c1ccc(Nc2nnc(-c3ccc(C(F)(F)F)cn3)o2)cn1 ; Cl] | >1 (no inhibition at 1 μM) |
| 70 | 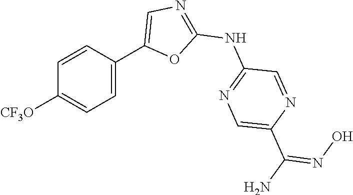 | <1 |
| 71 | 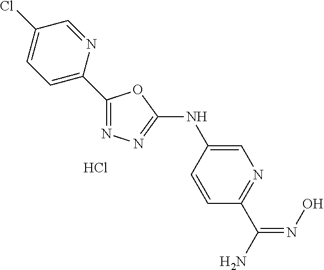 [SMILES: N/C(=N/O)c1ccc(Nc2nnc(-c3ccc(Cl)cn3)o2)cn1 ; Cl] | >1 (13% inhibition at 1 μM) |
| 72 | 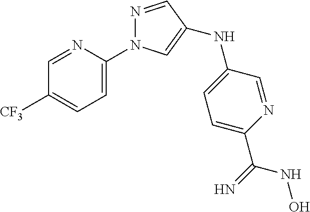 [SMILES: N=C(NO)c1ccc(Nc2cnn(-c3ccc(C(F)(F)F)cn3)c2)cn1] | <1 |
| 73 | 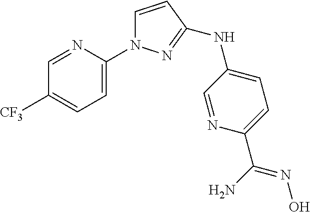 [SMILES: N/C(=N\O)c1ccc(Nc2ccn(-c3ccc(C(F)(F)F)cn3)n2)cn1] | <1 |
| 74 | 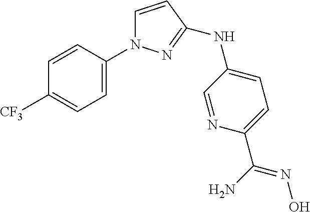 [SMILES: N/C(=N\O)c1ccc(Nc2ccn(-c3ccc(C(F)(F)F)cc3)n2)cn1] | <1 |
| 75 | 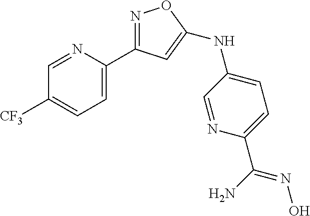 [SMILES: N/C(=N\O)c1ccc(Nc2cc(-c3ccc(C(F)(F)F)cn3)no2)cn1] | <1 |
| 76 | 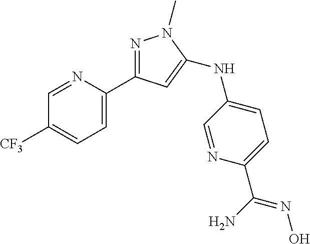 [SMILES: Cn1nc(-c2ccc(C(F)(F)F)cn2)cc1Nc1ccc(/C(N)=N/O)nc1] | >1 (8% inhibition at 1 μM) |
| 77 | 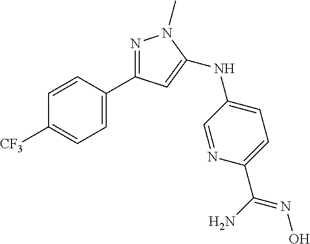 [SMILES: Cn1nc(-c2ccc(C(F)(F)F)cc2)cc1Nc1ccc(/C(N)=N/O)nc1] | >1 (47% inhibition at 1 μM) |
| 78 | 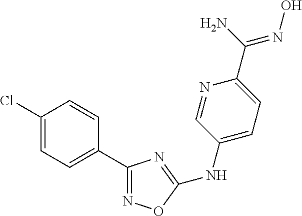 [SMILES: N/C(=N\O)c1ccc(Nc2nc(-c3ccc(Cl)cc3)no2)cn1] | <1 |
| 79 | 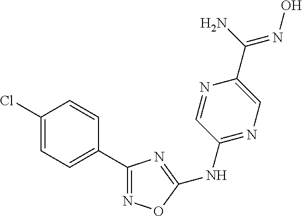 [SMILES: N/C(=N\O)c1cnc(Nc2nc(-c3ccc(Cl)cc3)no2)cn1] | <1 |
| 80 | 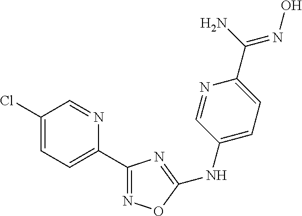 [SMILES: N/C(=N\O)c1ccc(Nc2nc(-c3ccc(Cl)cn3)no2)cn1] | >1 (24% inhibition at 1 μM) |
| 81 | 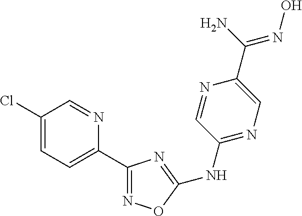 [SMILES: N/C(=N\O)c1cnc(Nc2nc(-c3ccc(Cl)cn3)no2)cn1] | >1 (7% inhibition at 1 μM) |
| 82 | 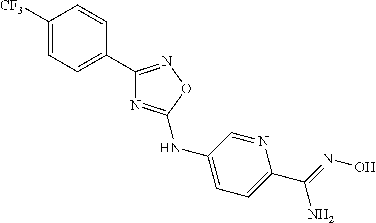 [SMILES: N/C(=N\O)c1ccc(Nc2nc(-c3ccc(C(F)(F)F)cc3)no2)cn1] | <1 |
| 83 | 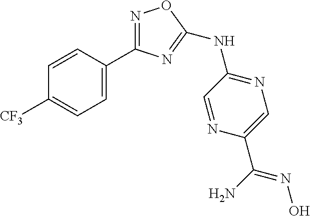 [SMILES: N/C(=N\O)c1cnc(Nc2nc(-c3ccc(C(F)(F)F)cc3)no2)cn1] | <1 |
| 84 | 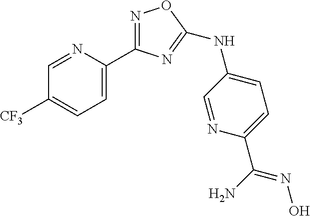 [SMILES: N/C(=N\O)c1ccc(Nc2nc(-c3ccc(C(F)(F)F)cn3)no2)cn1] | >1 (45% inhibition at 1 μM) |
| 85 | 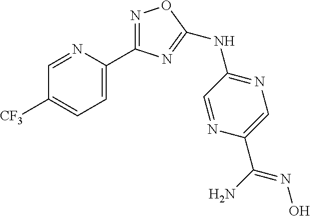 [SMILES: N/C(=N\O)c1cnc(Nc2nc(-c3ccc(C(F)(F)F)cn3)no2)cn1] | >1 (15% inhibition at 1 μM) |
| 86 | 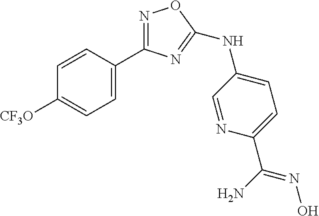 | <1 |
| 87 | 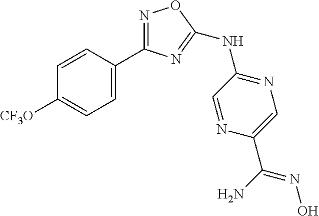 | 1 |
| 88 | 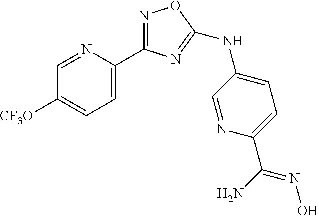 | >1 (29% inhibition at 1 μM) |
| 89 | 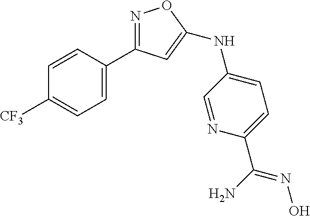 [SMILES: N/C(=N\O)c1ccc(Nc2cc(-c3ccc(C(F)(F)F)cc3)no2)cn1] | <1 |
| 90 | 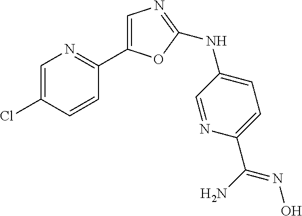 [SMILES: N/C(=N\O)c1ccc(Nc2ncc(-c3ccc(Cl)cn3)o2)cn1] | <1 |
| 91 | 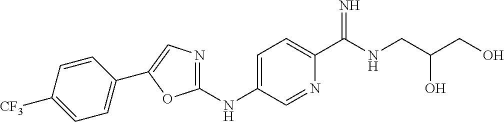 [SMILES: N=C(NCC(O)CO)c1ccc(Nc2ncc(-c3ccc(C(F)(F)F)cc3)o2)cn1] | >1 (44% inhibition at 1 μM) |
| 92 | 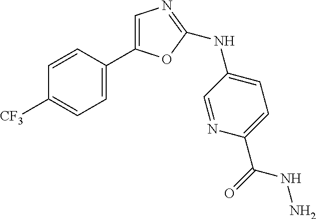 [SMILES: NNC(=O)c1ccc(Nc2ncc(-c3ccc(C(F)(F)F)cc3)o2)cn1] | 0.1-1.0 |
| 93 | 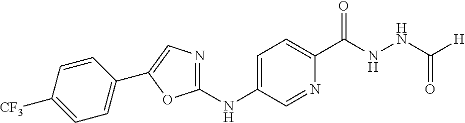 [SMILES: O=CNNC(=O)c1ccc(Nc2ncc(-c3ccc(C(F)(F)F)cc3)o2)cn1] | <1 |
| 94 | 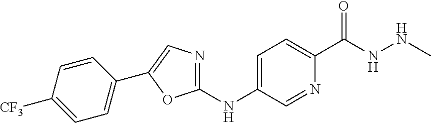 [SMILES: CNNC(=O)c1ccc(Nc2ncc(-c3ccc(C(F)(F)F)cc3)o2)cn1] | >1 (30% inhibition at 1 μM) |
| 95 | 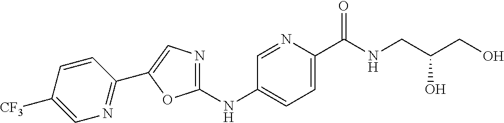 [SMILES: O=C(NC[C@@H](O)CO)c1ccc(Nc2ncc(-c3ccc(C(F)(F)F)cn3)o2)cn1] | 0.01-0.1 |
| 96 | 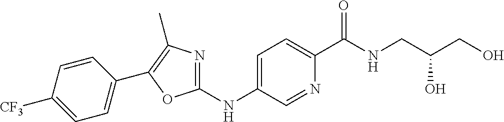 [SMILES: Cc1nc(Nc2ccc(C(=O)NC[C@@H](O)CO)nc2)oc1-c1ccc(C(F)(F)F)cc1] | <1 |
| 97 | 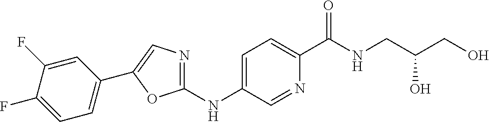 [SMILES: O=C(NC[C@@H](O)CO)c1ccc(Nc2ncc(-c3ccc(F)c(F)c3)o2)cn1] | >0.1 (55% inhibition at 0.1 μM) |
| 98 | 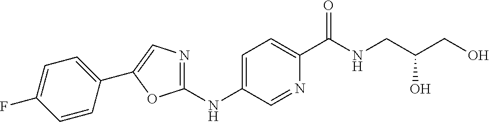 [SMILES: O=C(NC[C@@H](O)CO)c1ccc(Nc2ncc(-c3ccc(F)cc3)o2)cn1] | >0.1 (70% inhibition at 0.1 μM) |
| 99 | 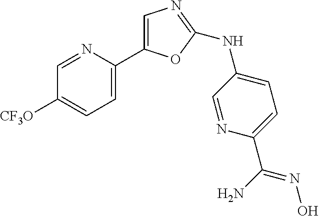 | >1 (47% inhibition at 1 μM) |
| 100 | 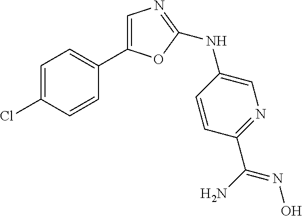 [SMILES: N/C(=N\O)c1ccc(Nc2ncc(-c3ccc(Cl)cc3)o2)cn1] | <1 |
| 101 | 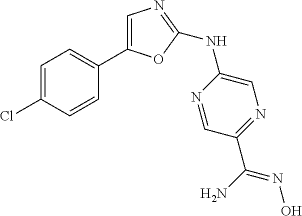 [SMILES: N/C(=N\O)c1cnc(Nc2ncc(-c3ccc(Cl)cc3)o2)cn1] | <1 |
| 102 | 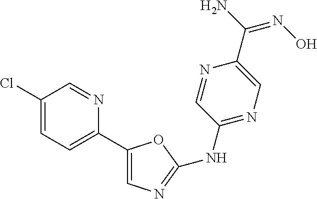 [SMILES: N/C(=N/O)c1cnc(Nc2ncc(-c3ccc(Cl)cn3)o2)cn1] | <1 |
| 103 | 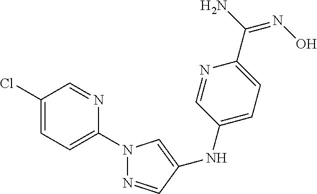 [SMILES: N/C(=N/O)c1ccc(Nc2cnn(-c3ccc(Cl)cn3)c2)cn1] | <1 |
| 104 | 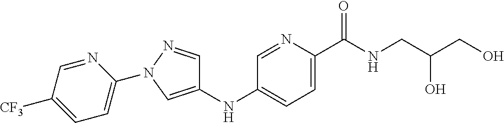 [SMILES: O=C(NCC(O)CO)c1ccc(Nc2cnn(-c3ccc(C(F)(F)F)cn3)c2)cn1] | >1 (26% inhibition at 1 μM) |
| 105 | 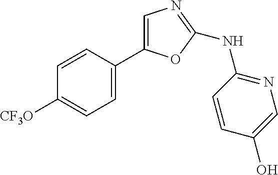 | <1 |
| 106 | 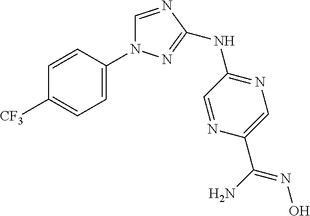 [SMILES: N/C(=N\O)c1cnc(Nc2ncn(-c3ccc(C(F)(F)F)cc3)n2)cn1] | <1 |
| 107 | 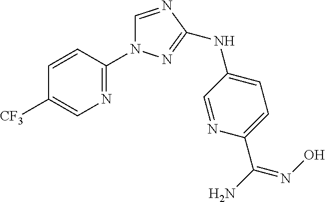 [SMILES: N/C(=N/O)c1ccc(Nc2ncn(-c3ccc(C(F)(F)F)cn3)n2)cn1] | <1 |
| 108 | 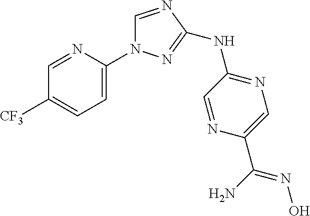 [SMILES: N/C(=N\O)c1cnc(Nc2ncn(-c3ccc(C(F)(F)F)cn3)n2)cn1] | <1 |
| 109 | 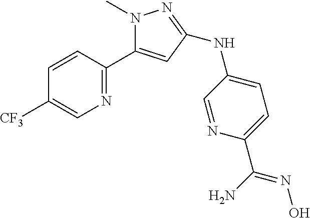 [SMILES: Cn1nc(Nc2ccc(/C(N)=N/O)nc2)cc1-c1ccc(C(F)(F)F)cn1] | <1 |
| 110 | 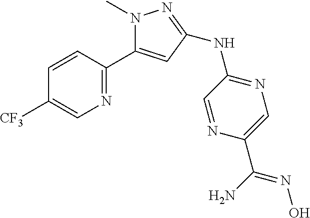 [SMILES: Cn1nc(Nc2cnc(/C(N)=N/O)cn2)cc1-c1ccc(C(F)(F)F)cn1] | >1 (31% inhibition at 1 μM) |
| 111 | 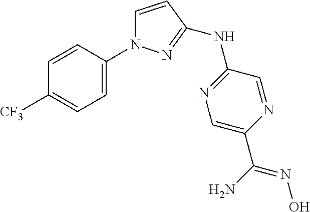 [SMILES: N/C(=N\O)c1cnc(Nc2ccn(-c3ccc(C(F)(F)F)cc3)n2)cn1] | <1 |
| 112 | 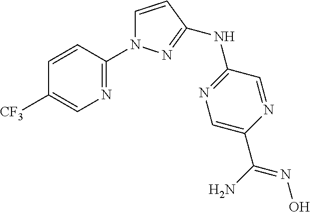 [SMILES: N/C(=N\O)c1cnc(Nc2ccn(-c3ccc(C(F)(F)F)cn3)n2)cn1] | <1 |
| 113 | 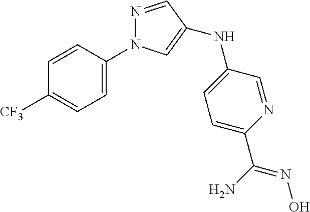 [SMILES: N/C(=N\O)c1ccc(Nc2cnn(-c3ccc(C(F)(F)F)cc3)c2)cn1] | <1 |
| 114 | 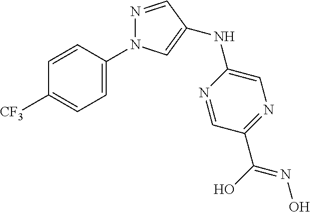 [SMILES: O/N=C(\O)c1cnc(Nc2cnn(-c3ccc(C(F)(F)F)cc3)c2)cn1] | <1 |
| 115 | 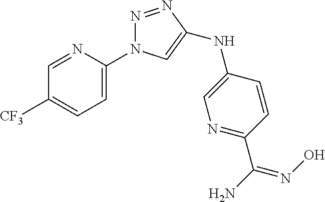 [SMILES: N/C(=N/O)c1ccc(Nc2cn(-c3ccc(C(F)(F)F)cn3)nn2)cn1] | <1 |
| 116 | 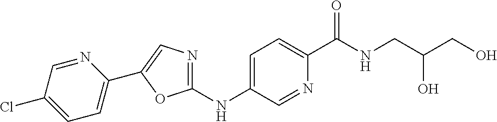 [SMILES: O=C(NCC(O)CO)c1ccc(Nc2ncc(-c3ccc(Cl)cn3)o2)cn1] | <1 |
| 117 | 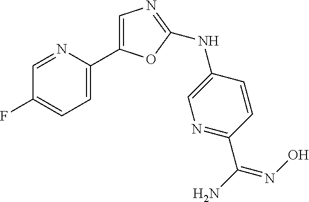 [SMILES: N/C(=N/O)c1ccc(Nc2ncc(-c3ccc(F)cn3)o2)cn1] | >1 (36% inhibition at 1 μM) |
| 118 | 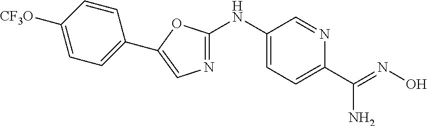 | <1 |
| 119 | 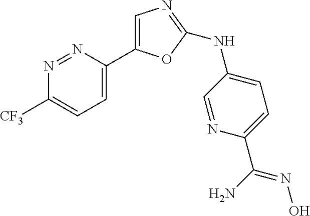 [SMILES: N/C(=N\O)c1ccc(Nc2ncc(-c3ccc(C(F)(F)F)nn3)o2)cn1] | >1 (44% inhibition at 1 μM) |
| 120 | 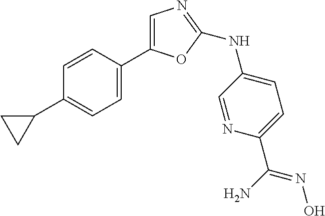 [SMILES: N/C(=N\O)c1ccc(Nc2ncc(-c3ccc(C4CC4)cc3)o2)cn1] | >1 (10% inhibition at 1 μM) |

^{a} IC₅₀ = concentration of test compound required to reduce Des1 activity to 50% of the vehicle control. Unless otherwise stated, this given as the concentration range of test compound within which the activity of Des1 was shown reduce to or below 50% of the vehicle control. If only measured at one concentration X (X = 10, 1, or 0.1 μM) the value is given as either <X (indicating that the Des1 activty at X is <50% of the vehicle control) or as >X (and the %-inhibition of Des1 at X is given).

### Example 3

In one or more embodiments, the presence of at least one ring nitrogen atom in W, e.g. in an ortho- and/or meta-position to the group R^{b}, may afford one or more surprising advantages, for example increased potency, improved selectivity, and/or one or more improved pharmacokinetic and/or physicochemical properties, e.g. absorption, distribution, metabolism and excretion, and solubility, compared to a W ring where nitrogen is absent, i.e. a phenyl group. Some non-limiting illustrative examples are presented below in Table 3-1.

TABLE 3-1

| | | | T_{1/2} Human Liver | |
|---|---|---|---|---|
| Compound | Des1 IC₅₀ | | Microsomes | |
| 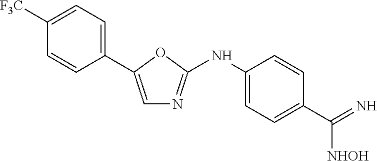 | 800-1000 | nM | 30-40 | min |
| (Reference Compound) | | | | |
| 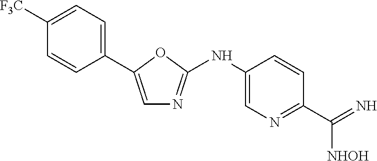 | 100-200 | nM | 90-100 | min |
| (Compound 8) | | | | |
| 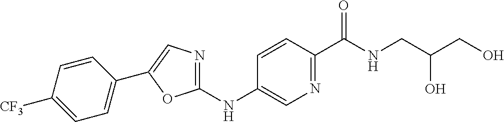 [SMILES: O=C(NCC(O)CO)c1ccc(Nc2ncc(-c3ccc(C(F)(F)F)cc3)o2)cn1] | 1-10 | nM | 210-220 | min |
| (Compound 17) | | | | |
| 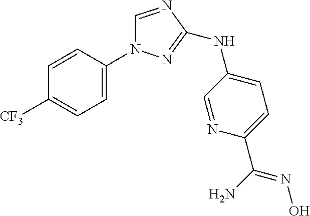 [SMILES: N/C(=N\O)c1ccc(Nc2ncn(-c3ccc(C(F)(F)F)cc3)n2)cn1] | 70-80 | nM | 170-180 | min |
| (Compound 24) | | | | |
| 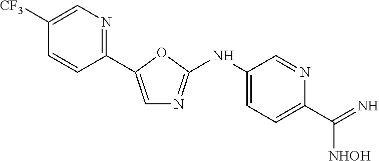 | 400-500 | nM | 190-200 | min |
| (Compound 46) | | | | |
| 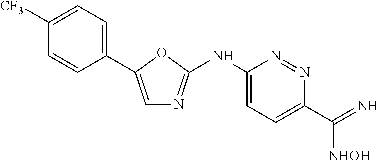 | 1-10 | nM | 150-170 | min |
| (Compound 53) | | | | |

### Example 4

### Real Time Quantitative Polymerase Chain Reaction in Human Hepatic Stellate Cells (LX2) to Detect Connective Tissue Growth Factor (CTGF) Gene Expression

The hepatic stellate cell line, LX2 was maintained in DMEM (LifeTechnologies) with 2% heat-inactivated FBS, 100 U penicillin (LifeTechnologies), 0.1 mg/mL streptomycin (LifeTechnologies), and 2 mM L-glutamine (LifeTechnologies) at 37° C. with 5% CO₂. Cells were seeded into 6-well plates (200,000 cells per well, 2 mLs media) and grown overnight. The cells were changed to 2 mLs 0.5% FBS media for 24 hours prior to treatment. Treatments were added in 2 mLs fresh 0.5% FBS media (vehicle control, 10 ng/mL TGFβ alone (R & D Systems), 10 ng/mL TGFβ+inhibitors) and incubated for 24 hours. Cells were washed once with cold PBS and lysed directly into RLT lysis buffer (Qiagen RNeasy kit, cat #74104, 350 L) containing 1% β-Mercaptoethanol. RNA was extracted using Qiagen RNeasy kit (cat #74104), according to manufacturer's instructions. RNA (1 g) was used to generate cDNA using the Qiagen Quantitect Reverse Transcription Kit (cat #205311), and stored at −20° C. cDNA was diluted (1:25 final) and combined with Sybr Green (Qiagen cat #204143), forward and reverse primers (50 ng each) and run on Real Time PCR Thermocycler machine (RG-6000, Corbett) with the following PCR conditions: Hold 1: 50° C. for 2 min; Hold 2: 95° C. for 15 min; Cycling: 45 cycles of: 95° C. for 30 sec, 58° C. for 21 sec, 72° C. for 15 sec, Hold 3: 72° C. for 30 sec, Melt: 72° C. to 99° C. GAPDH primers (Forward: ACC CAG AGG ACT GTG GAT GG; Reverse: CAG TGA GCT TCC CGT TCA G) CTGF primers (Forward: CTT GCG AAG CTG ACC TGG AAG A; Reverse: CCG TCG GTA CAT ACT CCA CAG A) [CTGF primers available from Origene, cat #HP205671)].

TABLE 4-1

| | | CTGF, LX2 % inhibition^{a} | | |
|---|---|---|---|---|
| Compound | Structure | 10 μM | 5 μM | 1 μM |
| 8 | 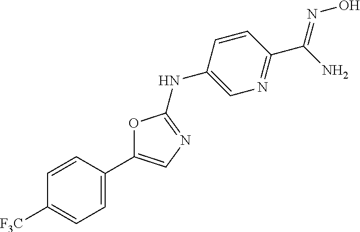 [SMILES: N/C(=N\O)c1ccc(Nc2ncc(-c3ccc(C(F)(F)F)cc3)o2)cn1] | — | >90 | — |
| 20 | 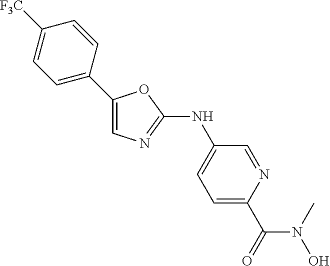 [SMILES: CN(O)C(=O)c1ccc(Nc2ncc(-c3ccc(C(F)(F)F)cc3)o2)cn1] | 70-80 | — | 10-20 |
| 23 | 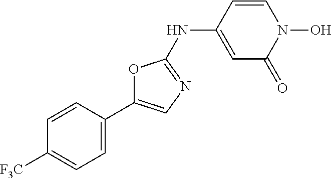 [SMILES: O=c1cc(Nc2ncc(-c3ccc(C(F)(F)F)cc3)o2)ccn1O] | >90 | — | 30-40 |
| 24 | 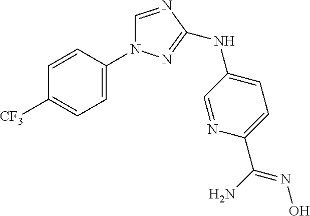 [SMILES: N/C(=N\O)c1ccc(Nc2ncn(-c3ccc(C(F)(F)F)cc3)n2)cn1] | — | 50-60 | — |
| 29 | 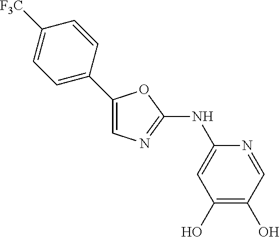 [SMILES: Oc1cnc(Nc2ncc(-c3ccc(C(F)(F)F)cc3)o2)cc1O] | >90 | — | >90 |
| 42 | 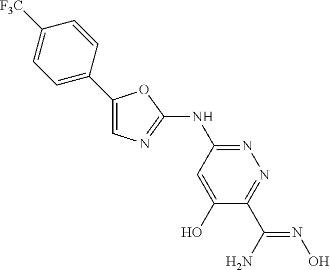 [SMILES: N/C(=N\O)c1nnc(Nc2ncc(-c3ccc(C(F)(F)F)cc3)o2)cc1O] | 40-50 | — | 10-20 |
| 46 | 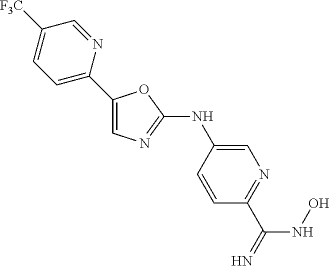 [SMILES: N=C(NO)c1ccc(Nc2ncc(-c3ccc(C(F)(F)F)cn3)o2)cn1] | — | >90 | — |
| 54 | 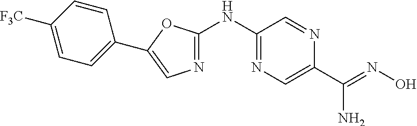 [SMILES: N/C(=N\O)c1cnc(Nc2ncc(-c3ccc(C(F)(F)F)cc3)o2)cn1] | 80-90 | — | 70-80 |
| 55 | 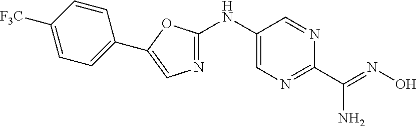 [SMILES: N/C(=N\O)c1ncc(Nc2ncc(-c3ccc(C(F)(F)F)cc3)o2)cn1] | 40-50 | — | 20-30 |
| 57 | 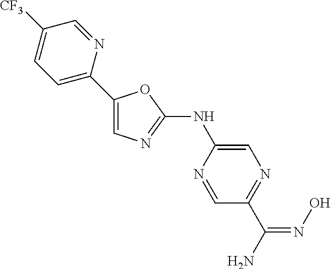 [SMILES: N/C(=N/O)c1cnc(Nc2ncc(-c3ccc(C(F)(F)F)cn3)o2)cn1] | 50-60 | — | 1-10 |
| 70 | 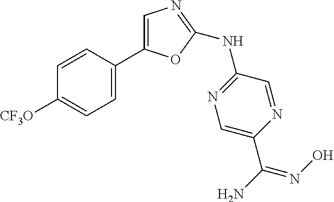 | — | >90 | — |
| 72 | 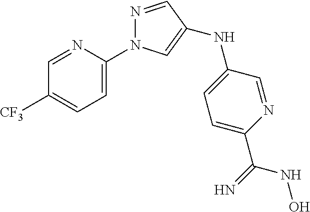 [SMILES: N=C(NO)c1ccc(Nc2cnn(-c3ccc(C(F)(F)F)cn3)c2)cn1] | — | 50-60 | 40-50 |
| 73 | 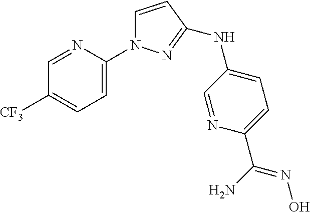 [SMILES: N/C(=N\O)c1ccc(Nc2ccn(-c3ccc(C(F)(F)F)cn3)n2)cn1] | — | >90 | — |
| 74 | 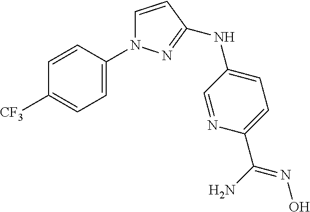 [SMILES: N/C(=N\O)c1ccc(Nc2ccn(-c3ccc(C(F)(F)F)cc3)n2)cn1] | — | 80-90 | — |
| 75 | 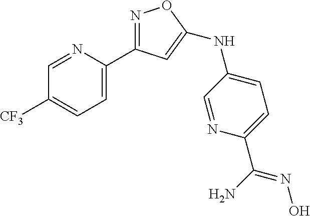 [SMILES: N/C(=N\O)c1ccc(Nc2cc(-c3ccc(C(F)(F)F)cn3)no2)cn1] | — | >90 | — |
| 82 | 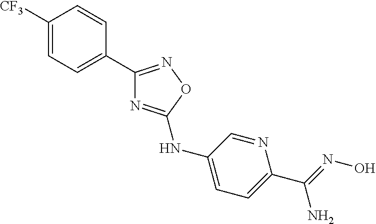 [SMILES: N/C(=N\O)c1ccc(Nc2nc(-c3ccc(C(F)(F)F)cc3)no2)cn1] | — | >90 | — |
| 84 | 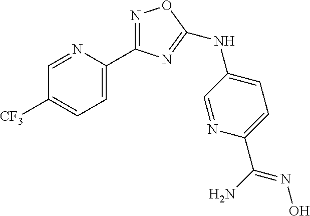 [SMILES: N/C(=N\O)c1ccc(Nc2nc(-c3ccc(C(F)(F)F)cn3)no2)cn1] | — | 80-90 | — |
| 86 | 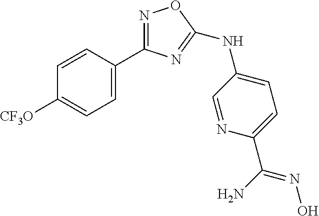 | — | >90 | — |
| 89 | 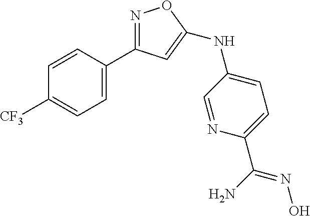 [SMILES: N/C(=N\O)c1ccc(Nc2cc(-c3ccc(C(F)(F)F)cc3)no2)cn1] | — | >90 | — |
| 90 | 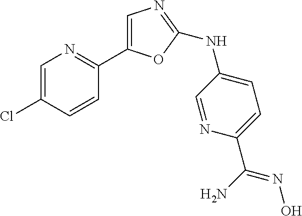 [SMILES: N/C(=N\O)c1ccc(Nc2ncc(-c3ccc(Cl)cn3)o2)cn1] | — | >90 | — |
| 99 | 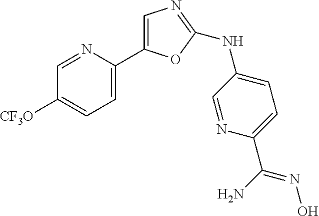 | 70-80 | — | 1-10 |
| 102 | 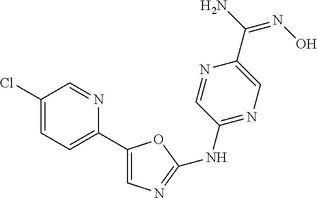 [SMILES: N/C(=N/O)c1cnc(Nc2ncc(-c3ccc(Cl)cn3)o2)cn1] | — | 70-80 | — |
| 103 | 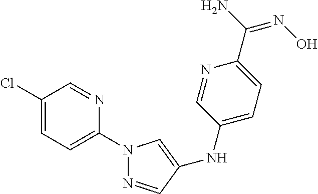 [SMILES: N/C(=N/O)c1ccc(Nc2cnn(-c3ccc(Cl)cn3)c2)cn1] | — | 70-80 | — |
| 106 | 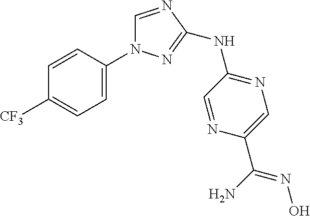 [SMILES: N/C(=N\O)c1cnc(Nc2ncn(-c3ccc(C(F)(F)F)cc3)n2)cn1] | — | — | 30-40 |
| 107 | 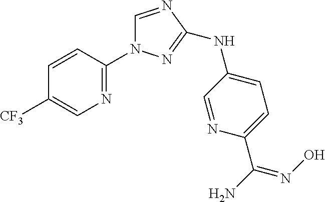 [SMILES: N/C(=N/O)c1ccc(Nc2ncn(-c3ccc(C(F)(F)F)cn3)n2)cn1] | — | >90 | — |
| 108 | 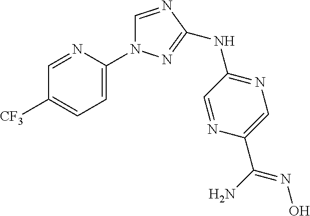 [SMILES: N/C(=N\O)c1cnc(Nc2ncn(-c3ccc(C(F)(F)F)cn3)n2)cn1] | — | 40-50 | — |
| 109 | 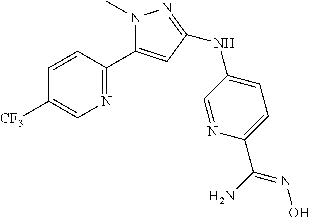 [SMILES: Cn1nc(Nc2ccc(/C(N)=N/O)nc2)cc1-c1ccc(C(F)(F)F)cn1] | — | >90 | — |
| 111 | 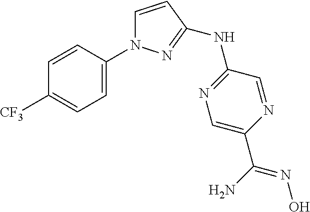 [SMILES: N/C(=N\O)c1cnc(Nc2ccn(-c3ccc(C(F)(F)F)cc3)n2)cn1] | — | >90 | — |
| 112 | 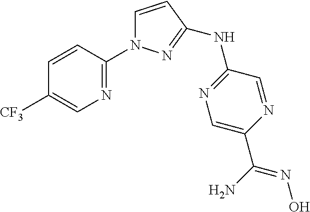 [SMILES: N/C(=N\O)c1cnc(Nc2ccn(-c3ccc(C(F)(F)F)cn3)n2)cn1] | — | — | >90 |
| 113 | 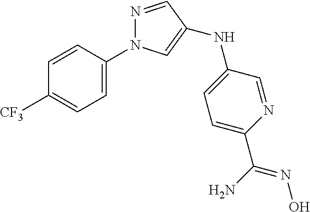 [SMILES: N/C(=N\O)c1ccc(Nc2cnn(-c3ccc(C(F)(F)F)cc3)c2)cn1] | — | >90 | — |
| 114 | 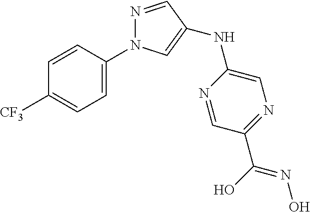 [SMILES: O/N=C(\O)c1cnc(Nc2cnn(-c3ccc(C(F)(F)F)cc3)c2)cn1] | — | >90 | — |
| 115 | 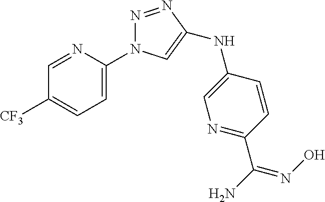 [SMILES: N/C(=N/O)c1ccc(Nc2cn(-c3ccc(C(F)(F)F)cn3)nn2)cn1] | — | >90 | — |
| 116 | 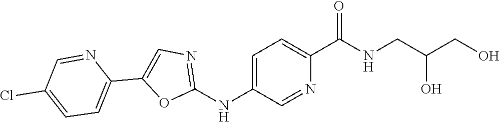 [SMILES: O=C(NCC(O)CO)c1ccc(Nc2ncc(-c3ccc(Cl)cn3)o2)cn1] | — | 70-80 | — |
| 118 | 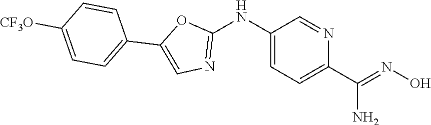 | 70-80 | — | 50-60 |
| 120 | 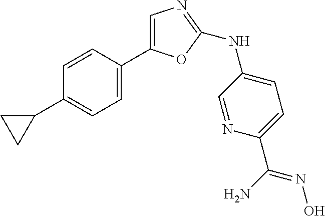 [SMILES: N/C(=N\O)c1ccc(Nc2ncc(-c3ccc(C4CC4)cc3)o2)cn1] | 40-50 | — | 40-50 |

^{a}% inhibition = the percentage inhibition of CTGF mRNA levels in TGFβ-stimulated LX2 cells at 10, 5, 3 or 1 μM of test compounds was calculated with reference to the vehicle control (TBGβ-stimulated LX2 cells in the absence of test compounds).

### Example 5

### Inhibition of Collagen Synthesis in Rat Mesangial Cells (RMCs)

Mesangial cells at passage (P36, P39 and P39 for each run respectively) were be plated into 24 well dishes at ˜15,000 cells/well. Cells were be allowed to adhere overnight, aiming for 60% confluence the next day. Actual reported confluence for each run was 90% by day 2. Cells were serum starved overnight in DMEM with 150 μM L-ascorbic acid, in the presence of 0.1% BSA. The following day the media was replaced with fresh starve medium containing Des1 inhibitor compounds at the doses of 0.01, 0.1, 1, 3, 10 μM each and incubated for 4 hrs before adding TGF-β (5 ng/ml, PeproTech) and 1 μCi/mL ³H-proline (Amersham L-(2,3,4,5-3H)-proline, 75 Ci/mmol) for proline incorporation. Finally, cells were incubated for a further 44 hours. At the end of the incubation time, cells were be collected by aspirating the supernatant and washing cells 3 times with ice cold Phosphate Buffered Saline (PBS). Ice cold 10% of 1 ml Trichloroacetic acid (TCA)/well is added to each well and cells will be incubated on ice for 30 minutes. Cells were then washed with 1 ml ice cold 10% TCA and solubilised over night at 4° C. in 0.75 ml 1 M Sodium Hydroxide (NaOH). 0.5 ml aliquots of solubilised cells were transferred to scintillation vials, neutralised with an equal volume of 1 M Hydrochloric Acid (HCl) and 10 ml of Instagel scintillant added. Counts were measured on a beta counter (PerkinElmer, Rowville, Australia). Proline incorporation is adjusted for protein content. An aliquot of the remaining lysate will be neutralised with 1 M HCl and assayed in a BioRad (Bradford) Protein Assay. Results will be expressed as cpm ³H-proline/μg total protein. A standard curve of BSA containing an equivalent amount of NaOH and HCl as the samples is prepared Compounds 8 and 46 were tested at 5 doses in three independent experiments. Within each run, 3 technical replicates of each compound and dose were performed.

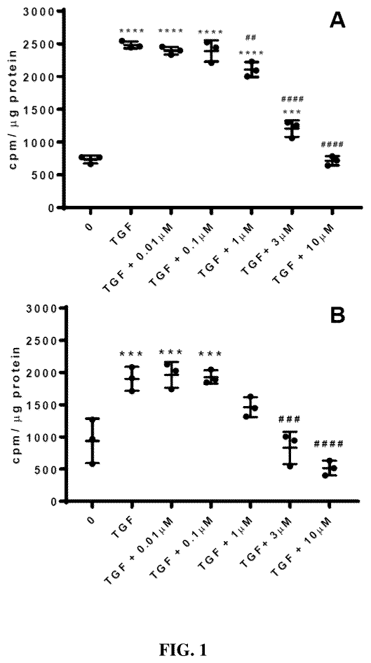

The results are depicted in FIGS. 1A and 1B.

Fibroblast-to-Myofibroblast Transition (FMT) in Human Lung Cells Derived from Idiopathic Pulmonary Fibrosis (IPF) Patient Donors

Lung-derived primary human bronchial fibroblasts (from IPF donors) were seeded at 3,000 cells per well in 96-well plates, and were grown over 5 days prior to treatment with the desired compounds (cell medium was refreshed at 48 h). Cells were then treated with 8 different concentrations of Compound 8 or 46, in semi-log dilutions starting from the top dosage of 10 μM (vehicle control: 0.1% DMSO; positive control: SB525334 and nintedanib). 1-hour post compound addition, the cells were stimulated with the addition of 1.25 ng/mL TGF-01, and incubated for 72 hours. Cells were then fixed with 4% formaldehyde, then stained using DAPI-labeled αSMA and imaged via high-content analysis. The assay was conducted in biological duplicates. This in vitro FMT assay results were provided by Charles River Laboratories through a service agreement.

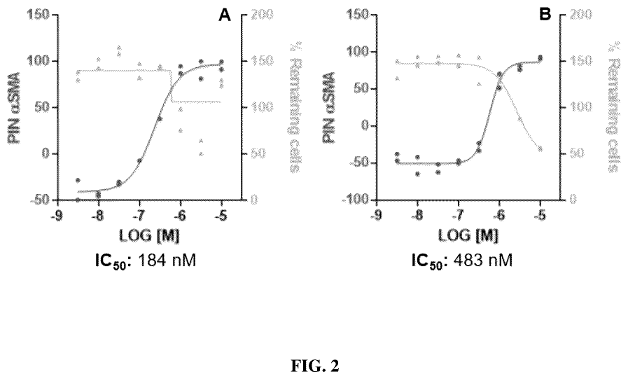

The results are depicted in FIGS. 2A and 2B.

1. A compound of formula (I′): 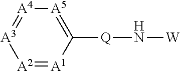
wherein:
A¹-A⁵ are independently selected from C—R^{a} and N, wherein any 0, 1, 2, 3 or 4 of A¹-A⁵ may be N;
each R^{a} is independently H or R^{aa}, wherein R^{aa} is selected from halo, alkyl, haloalkyl, cycloalkyl, halocycloalkyl, alkoxy, haloalkoxy, cycloalkoxy, cycloamino, halocycloamino, alkoxylalkyl, and alkoxyalkoxy;
Q is a 5-membered heteroaromatic ring having 2, 3 or 4 ring heteroatoms, at least one of which must be N, and the remaining are independently selected from N, O and S, and wherein a ring carbon atom bearing a hydrogen atom, or a ring nitrogen atom bearing a hydrogen atom, if present, may be optionally substituted with Q^{a}, which is selected from halo, haloalkyl and alkyl;
W is a 6-membered N-containing heterocycle selected from: 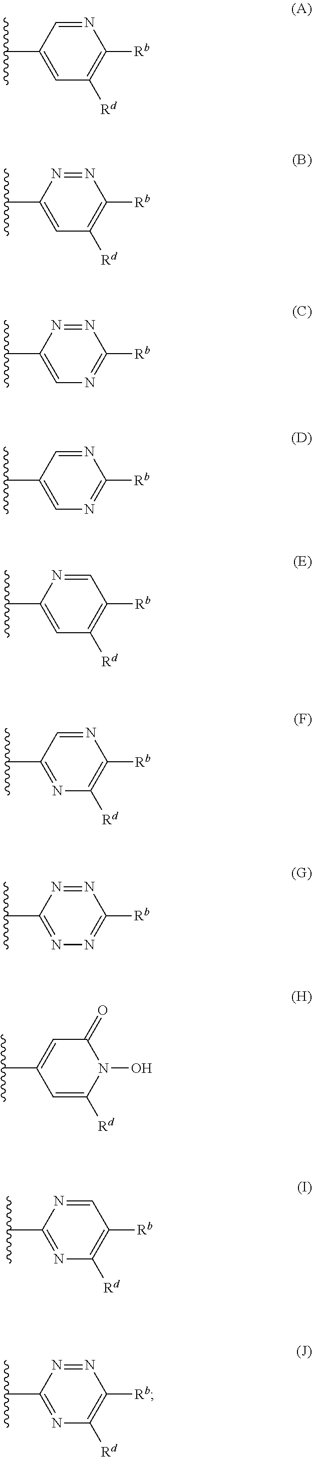
wherein
R^{b} is selected from:
—OH, provided that when R^{b} is OH, W is (E) or (I);
—K—NR^{c}—Y, or a tautomer thereof,
—(CH₂)ₚ—NH—OH, and 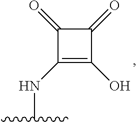
wherein
K is SO, SO₂, C(═X′) or NHC(═X′), where X′ is O or NH;
R^{c} is H, C₁₋₆ alkyl; or hydroxyC₁₋₆ alkyl;
Y is OH, NHR^{e} (wherein R^{e} is H, C₁₋₆ alkyl, —(C═O)H or —(C═O)C₁₋₆ alkyl), hydroxyC₁₋₆ alkyl or (SC₁₋₆ alkyl)C₁₋₆ alkyl; and
p is 0 or 1; and
R^{d} is selected from H, OH, halo, C₁₋₆ alkyl and C₁₋₆ alkoxy;
provided that the compound does not have the formula 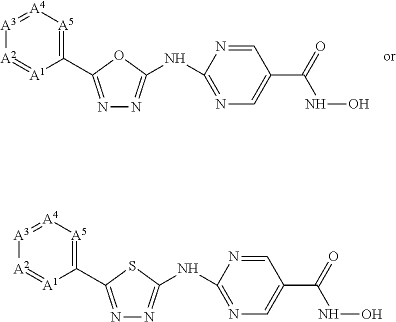
where any four of A¹-A⁵ are C—H, and the other is C—R^{a} where R^{a} is H, halo, CH₃ or OCH₃;
or a pharmaceutically acceptable salt or solvate thereof.

2. The compound according to claim 1 wherein Q is selected from heterocyclic formulas (a)-(kk), which may optionally substituted with a group Q^{a} where permissible (where the bonds labelled # are attached to NH and the bonds labelled * are attached to the aryl ring defined by A¹-A⁵):

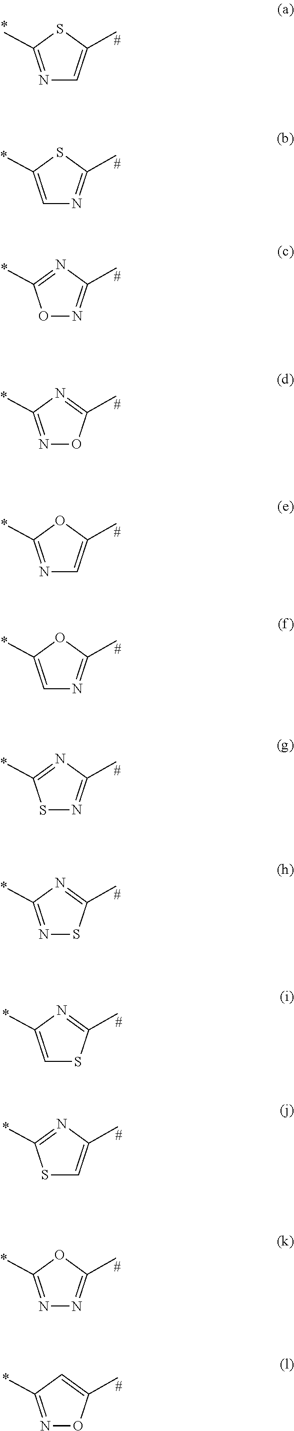

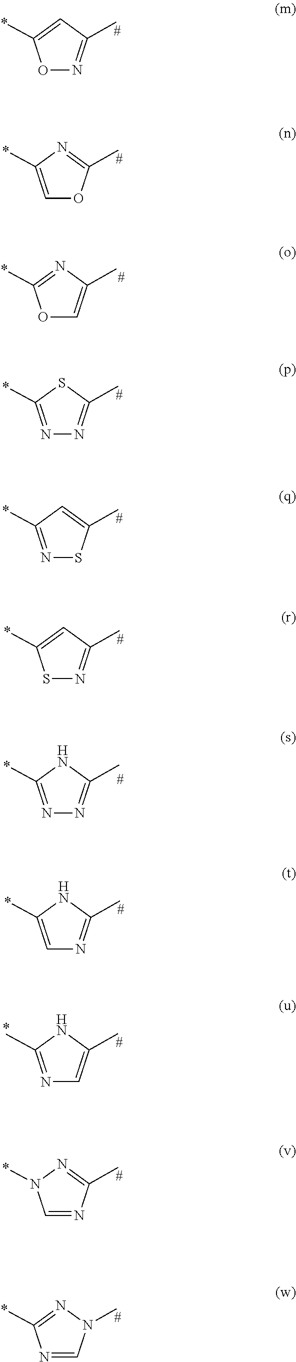

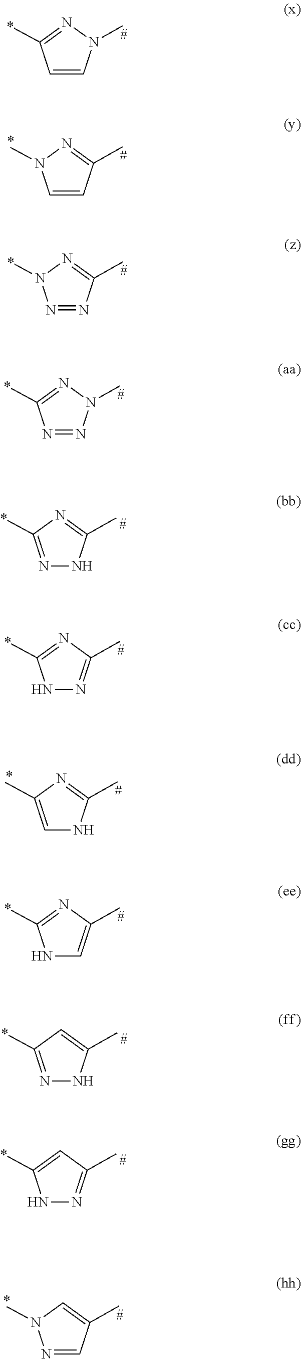

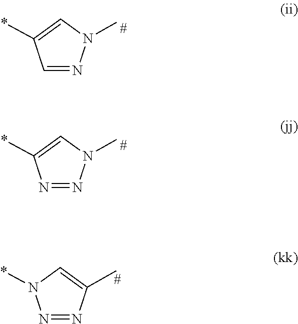

3. The compound according to claim 1 wherein Q^{a} is selected from halo, C₁₋₆ alkyl, and halo C₁₋₆ alkyl.

4. The compound according to claim 1 wherein each of A¹-A⁵ is C—R^{a}.

5. The compound according to claim 1 wherein 1, 2, 3 or 4 of A¹, A², A⁴ and A⁵ are N.

6. The compound according to claim 5 wherein
A⁵ or A¹ is N; or
A² or A⁴ is N; or
A¹ and A⁵ are both N; or
A² and A⁴ are both N
A¹ and A⁴, or A² and A⁵ are both N; or
A¹ and A² or A⁴ and A⁵ are both N.

7. The compound according to claim 1 wherein A³ is C—R^{aa}.

8. The compound according to claim 7 where R^{aa} is halo, haloalkyl or haloalkoxy.

9. The compound according to claim 8 wherein R^{aa} is Cl, CF₃ or OCF₃.

10. The compound according to a claim 1 wherein W is selected from: (A), (B), (D), (E), (F), (H) and (I).

11. The compound according to claim 1 where W is selected from (A), (B), (C), (D), (F), (G) (H) and (J).

12. The compound according to claim 1 where Q is (f), optionally substituted with Q^{a}.

13. The compound according to claim 1 where W is (A).

14. The compound according to claim 1 wherein R^{d} is H, OH, Cl, F, Br, I, CH₃ or OCH₃.

15. The compound according to claim 1 wherein R^{b} is K—NR^{c}—Y, or a tautomer thereof.

16. The compound according to a claim 1 wherein R^{b} is —K—NR^{c}—Y, or a tautomer thereof, wherein:
K is SO₂, C(═O), C(═NH), or NHC(═O),
R^{c} is H, C₁₋₆ alkyl (e.g. C₁₋₃ alkyl, such as CH₃, CH₂CH₃ or (CH₂)₂CH₃); or hydroxyC₁₋₆ alkyl (e.g. hydroxyC₁₋₃ alkyl; such as —(CH₂)OH, —(CH₂)₂OH, —CH(OH)CH₂OH, CH(OH)CH₃, —(CH₂)₃OH, —CH(OH)(CH₂)₂OH, —CH₂CH(OH)CH₂OH, —(CH(OH))₂CH₃ and —(CH(OH))₂CH₂OH); and
Y is OH, NH₂, NHC₁₋₃ alkyl, NHC(═O)H, NH(C═O)C₁₋₃ alkyl, hydroxyC₁₋₆ alkyl (e.g. hydroxyC₁₋₃ alkyl; such as —(CH₂)OH, —(CH₂)₂OH, —CH(OH)CH₂OH, CH(OH)CH₃, —(CH₂)₃OH, —CH(OH)(CH₂)₂OH, —CH₂CH(OH)CH₂OH, —(CH(OH))₂CH₃ and —(CH(OH))₂CH₂OH); or (SC₁₋₆ alkyl)C₁₋₆ alkyl (e.g. (SC₁₋₃ alkyl)C₁₋₃ alkyl, such as —(CH₂)SCH₃, —(CH₂)₂SCH₃, —CH(SCH₃)CH₂SCH₃, CH(SCH₃)CH₃, —(CH₂)₃SCH₃, —CH(SCH₃)(CH₂)₂SCH₃, —CH₂CH(SCH₃)CH₂SCH₃, —(CH(SCH₃))₂CH₃ and —(CH(SCH₃))₂CH₂SCH₃).

17. The compound according to a claim 1 wherein R^{b} is 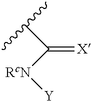
wherein
X′ is O or NH,
R′ is H or C₁₋₆ alkyl; or hydroxyC₁₋₆ alkyl; and
Y is OH or NHR^{e},
or a tautomer thereof

18. The compound according to claim 1 wherein R^{b} is selected from: 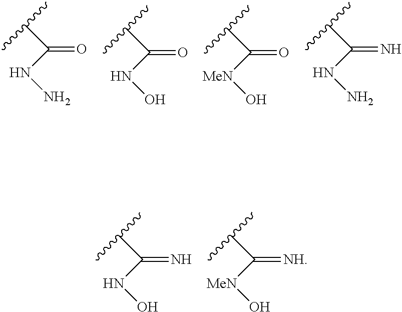 [SMILES: *C(=O)NN ; *C(=O)NO ; *C(=O)N(C)O ; *C(=N)NN ; *C(=N)NO ; *C(=N)N(C)O]
or a tautomer thereof.

19. The compound according to claim 1 wherein R^{b} is:

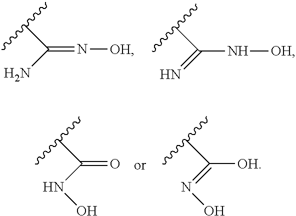 [SMILES: *C(N)=NO ; *C(=N)NO ; *C(=O)NO ; */C(O)=N/O]

20. The compound according to claim 1 wherein R^{b} is OH and W is (E) or (I).

21. A compound according to claim 1 which is any one of Compounds 1-120 described herein.

22. A composition comprising a compound according to claim 1, or a pharmaceutically acceptable salt or solvate thereof, and a pharmaceutically acceptable additive.

23. A compound according to claim 1, for use as an agent for inhibiting or otherwise interacting with Des1 or for use as an agent in treating fibrosis or a fibrotic disease.

24. A compound according to claim 1 for use in therapy.

25. A method of treating a disease or condition in which Des1 inhibition is beneficial, in a subject in need thereof, comprising administering to said subject, compound according to claim 1, or a pharmaceutically acceptable salt or solvate thereof.

26. Use of a compound according to claim 1, or a pharmaceutically acceptable salt or solvate thereof, in the manufacture of a medicament for treating a disease in which Des1 inhibition is beneficial.

27. A method of treating a fibrosis or a fibrotic disease in a subject in need thereof, comprising administering to said subject, compound according to claim 1, or a pharmaceutically acceptable salt or solvate thereof.

28. Use of a compound according to claim 1, or a pharmaceutically acceptable salt or solvate thereof, in the manufacture of a medicament for treating fibrosis or a fibrotic disease.
